(19) United States
(12) Patent Application Publication
(10) Pub. No.: US 2026/0015617 A1
(43) Pub. Date: Jan. 15, 2026

(54) DOUBLE STRANDED RNAi AGENTS, COMPOSITIONS AND METHODS OF USE

(71) Applicant: Novartis AG, Basel (CH)

(72) Inventors: Margaret Elizabeth BROUSSEAU, Dracut, MA (US); Shaun Robert COUGHLIN, Cambridge, MA (US); Payel GHATAK, Westwood, MA (US); Stefan HAEMMIG, Bottmingen (CH); Caitlin Jeanette HAGEN, Wilmington, MA (US); Zhihong HUANG, San Diego, CA (US); Juerg HUNZIKER, Aarau (CH); Ge TAN, Bottmingen (CH); Jan WEILER, Lorrach (DE)

(21) Appl. No.: 19/265,019

(22) Filed: Jul. 10, 2025

Related U.S. Application Data
(60) Provisional application No. 63/670,342, filed on Jul. 12, 2024.
(60) Provisional application No. 63/786,760, filed on Apr. 10, 2025.

(51) Int. Cl. C12N 15/113; A61K 9/00; A61K 45/06; A61P 3/06

(52) U.S. Cl. CPC C12N 15/1137; A61K 9/0019; A61K 45/06; A61P 3/06; C12N 2310/11; C12N 2310/14; C12N 2310/312; C12N 2310/315; C12N 2310/321; C12N 2310/322; C12N 2310/351; C12N 2320/31; C12Y 101/01034; C12Y 304/21061

(57) ABSTRACT

Disclosed are, inter alia, double stranded RNAi (dsRNAi) agents inhibiting expression of 3-hydroxy-3-methylglutaryl-CoA reductase (HMGCR), for example, human HMGCR, compositions including the same, and methods of treatment using the same.

### CROSS-REFERENCE TO RELATED APPLICATIONS

The present application claims priority and benefit to the U.S. Patent Application No. 63/670,342 filed Jul. 12, 2024 and U.S. Patent Application No. 63/786,760 filed Apr. 10, 2025, the disclosure of each of which is incorporated herein by reference in its entirety.

### SEQUENCE LISTING

The instant application contains a Sequence Listing which has been submitted electronically in XML format and is hereby incorporated by reference in its entirety. Said XML copy, created on Jul. 1, 2025, is named PAT059649-US-NP_SL.xml and is 23,177,182 bytes in size.

### TECHNICAL FIELD

The present disclosure provides, inter alia, double stranded RNAi (dsRNAi) agents inhibiting expression of 3-hydroxy-3-methylglutaryl-CoA reductase (HMGCR), for example, human HMGCR, compositions including the same, and methods of treatment using the same.

### BACKGROUND

Cumulative low-density lipoprotein cholesterol (LDL-C) exposure in the arterial wall is a major cause of atherosclerotic cardiovascular disease (ASCVD). The level of LDL-C in the arterial wall can be controlled (e.g., lowered) by modulating cholesterol homeostasis (e.g., cholesterol biosynthesis in liver cells) and upregulating LDL-C uptake from the blood.

3-Hydroxy-3-methylglutaryl-CoA reductase (HMGCR) is a key enzyme that converts 3-hydroxy-3-methyl-glutaryl-CoA (HMGCoA) into mevalonate in the rate-limiting step in the cholesterol biosynthesis pathway. Due to its critical role in cholesterol biosynthesis, HMGCR has been a target for drug development for the treatment of high cholesterol and cardiovascular disease risk reduction (CVRR). For example, statins, small molecule inhibitors of HMGCR, are the current standard of care for lowering cholesterol. However, statins also have adverse side effects such as myalgia and rhabdomyolysis, a rare, but potentially fatal, breakdown of skeletal muscle. Real-world studies demonstrate that a staggering 80% of US patients on standard of care do not achieve their LDL-C goal. This is due, in large part, to poor adherence.

Therefore, there is an unmet need in the art for alternative treatments (e.g., lowering cholesterol or LDL-C) for subjects having lipid metabolism disorders.

### SUMMARY OF THE INVENTION

Provided herein are, inter alia, compounds that can inhibit expression of HMGCR in a subject, for example, e.g., in liver cells of a subject.

In an aspect, provided is a double stranded RNAi (dsRNAi) agent comprising:
a sense strand comprising a nucleotide sequence selected from SEQ ID NOs: 1 to 405 and 1434 to 1440 in Table 1; and
an antisense strand forming a duplex with the sense strand and comprising a nucleotide sequence selected from SEQ ID NOs: 406 to 810 and 1441 to 1447 in Table 1.

In some embodiments, the sense strand is 21 to 23 nucleotides in length and the antisense strand is 23 to 25 nucleotides in length.

In some embodiments, all the nucleotides in the sense strand and the antisense strand are modified nucleotides.

In some embodiments, each of the modified nucleotides independently comprises one or more modifications selected from a 2′-deoxy modification, a 2′-O-alkyl modification, a 2′-halo modification, a threofuranosyl nucleotide (TNA) modification, a 2′-5′-linkage modification, a conformationally restricting modification, an abasic modification, a 2′-amino-modification, a 2′-O-allyl modification, 2′-C-alkyl modification, a 2′-O-alkoxyalkyl modification, a morpholino modification, a phosphoramidate modification, a non-natural nucleobase modification, a modification in a tetrahydropyran, a modification containing a 1,5-anhydrohexitol, a modification containing a cyclohexenyl, a modification containing a phosphorothioate group, a modification containing a 5′-vinyl-phosphonate, a modification containing a 5′-phosphate, a modification to form a thermally destabilizing nucleotide, a glycol nucleic acid (GNA) modification, and a 2-O-(N-methylacetamide) modification.

In some embodiments, each of the modified nucleotides independently comprises one or more modifications selected from 2′-deoxy modification, 2′-O-alkoxyalkyl modification, 2′-O-alkyl modification, 2′-O-allyl modification, 2′-C-allyl modification, 2′-halo modification, modification containing a non-natural nucleobase, GNA modification, and TNA modification.

In some embodiments, all the modified nucleotides comprise a modification on a 2′ sugar ring.

In some embodiments, the modified nucleotides are selected from a 2′-O-alkyl modified nucleotide, a 2′-halo modified nucleotide, a 2′-deoxy modified nucleotide, a 2′-O-alkoxyalkyl modified nucleotide and TNA modification.

In some embodiments, one or more of the modified nucleotides further comprises a 3′-phosphorothioate (PS) modification.

In some embodiments, each of the modified nucleotides independently comprises one or more modifications selected from 2′-deoxy modification, 2′-O-methyl (2′-OMe) modification, 2′-fluoro (2′-F) modification, 2′-O-methoxyethyl (2′-MOE) modification, the modification containing a non-natural nucleobase, TNA, GNA, 3′-phosphorothioate (PS) modification, and 5′-vinyl-phosphonate (5′-VP) modification.

In some embodiments, the sense strand comprises one or two 2′-MOE modified nucleotides positioned at the 1^{st} and/or 2^{nd} nucleotides from the 5′-end of the sense strand.

In some embodiments, the sense strand comprises one or two 2′-MOE modified nucleotides positioned at the 1^{st} and/or 2^{nd} nucleotides from the 3′-end of the sense strand.

In some embodiments, the sense strand comprises one or two TNAs positioned at the 1^{st} and/or 2^{nd} nucleotides from the 5′-end of the sense strand.

In some embodiments, the sense strand comprises one or two TNAs positioned at the 1^{st} and/or 2^{nd} nucleotides from the 3′-end of the sense strand.

In some embodiments, the antisense strand comprises a 5′-VP group at the 1^{st} nucleotide from 5′ end of the antisense strand.

In some embodiments, the antisense strand comprises a 5′-(E)-VP group at the 1^{st} nucleotide from 5′ end of the antisense strand.

In some embodiments, the antisense strand comprises a 5′-(E)-VP-2′-OMe nucleotide at the 1^{st} position from 5′ end of the antisense strand.

In some embodiments, each of the sense strand and the antisense strand independently comprises two, three, four, five or six 2′-F modified nucleotides.

In some embodiments, the sense strand comprises one or two 3′-PS group at the 1^{st} and/or 2^{nd} nucleotides from 5′-end of the sense strand.

In some embodiments, the antisense strand comprises one or two 3′-PS group at the 1^{st} and/or 2^{nd} nucleotides from 5′-end of the antisense strand, and/or one or two 3′-PS group at the 1^{st} and/or 2^{nd} nucleotides from 3′-end of the antisense strand.

In some embodiments, the sense strand is 21 nucleotides in length and the antisense strand is 23 nucleotides in length.

In some embodiments, the sense strand comprises one to four 2′-MOE modified nucleotides positioned at the 1^{st}, 2^{nd}, 20^{th}, and/or 21^{st} nucleotides from the 5′-end of the sense strand.

In some embodiments, the sense strand comprises only four 2′-MOE modified nucleotides.

In some embodiments, the sense strand does not comprise a 2′-MOE modified nucleotide at the 3^{rd} to 19^{th} positions from 5′-end of the sense strand.

In some embodiments, the sense strand comprises one to four TNAs positioned at the 1^{st}, 2^{nd}, 20^{th}, and/or 21^{st} nucleotides from the 5′-end of the sense strand.

In some embodiments, the sense strand comprises two, three, or four 2′-F modified nucleotides positioned at the 7^{th}, 9^{th}, 10^{th}, and/or 1^{th} nucleotide from 5′-end of the sense strand.

In some embodiments, the sense strand comprises 2′-F modified nucleotides positioned at the 7^{th}, 9^{th}, 10^{th}, and 11^{th} nucleotides from 5′-end of the sense strand.

In some embodiments, the remaining nucleotides in the sense strand comprise 2′-OMe modified modification.

In some embodiments, the antisense strand comprises a 5′-(E)-VP group at the 1^{st} nucleotide from 5′ end of the antisense strand.

In some embodiments, the antisense strand comprises two, three, or four 2′-F modified nucleotides positioned at the 2^{nd}, 6^{th}, 14^{th}, and/or 16^{th} nucleotides from 5′-end of the antisense strand.

In some embodiments, the antisense strand comprises 2′-F modified nucleotides positioned at the 2^{nd}, 6^{th}, 14^{th}, and 16^{th} nucleotides from 5′-end of the antisense strand.

In some embodiments, the antisense strand comprises 2′-F modifications positioned at the 2nd, 6th, 14th, and 16th nucleotides from the 5′ end; and (i) a GNA positioned at the 5^{th} nucleotide from 5′ end, or (ii) a TNA positioned at the 3rd nucleotide from the 5′ end.

In some embodiments, the remaining nucleotides in antisense strand comprise 2′-OMe modified modifications.

In some embodiments, the sense strand comprises one to eight 3′-PS group at the 1^{st} 2^{nd}, 3^{rd}, 4^{th}, 17^{th}, 18^{th}, 19^{th} and/or 20^{th} nucleotides from 5′-end of the sense strand.

In some embodiments, the antisense strand comprises one to eight 3′-PS group at the 1^{st}, 2^{nd}, 3^{rd}, 4^{th}, 19^{th}, 20^{th}, 21^{st} and/or 22^{nd} nucleotides from 5′-end of the antisense strand.

In some embodiments, at least one of the 3′-PS groups in each sense strand and antisense strand has a stereopure Rp configuration.

In some embodiments, at least one of the 3′-PS groups in each sense strand and antisense strand has a stereopure Sp configuration.

In certain aspects, provided is a double stranded RNAi (dsRNAi) agent comprising:
a sense strand having a nucleotide sequence selected from SEQ ID NOs: 812 to 1052 in Table 2 and SEQ ID NOs: 1294 to 1297, 1448 to 1462, and 1481 to 1482 in Table 3; and
an antisense strand forming a duplex with the sense strand and having a nucleotide sequence selected from SEQ ID NOs: 1053 to 1293 in Table 2 and 1298 to 1301, 1463 to 1477, and 2600 to 2605 in Table 3.

In some embodiments, the dsRNAi comprises a ligand.

In some embodiments, the ligand comprises a N-acetylgalactosamine (GalNAc) moiety.

In some embodiments, the ligand has a structure of:

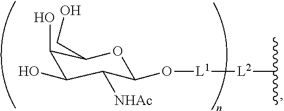

wherein:
each L¹ is independently a linker which may be same or different in each occurrence;
L² is a linker;
n is an integer from 1 to 3; and
[symbol] is an attachment point to the sense strand or an antisense strand.

In some embodiments, the ligand comprises the following structure of

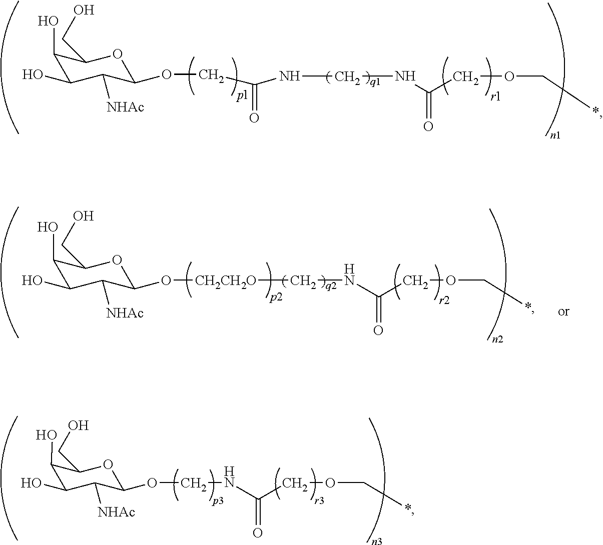

wherein:
each p1, p2, p3, q1, q2, r1, r2 and r3 is independently an integer from 0 to 12;
each n1, n2, and n3 is independently an integer from 1 to 3; and
“*” is an attachment point to L².

In some embodiments, the ligand has a structure of:

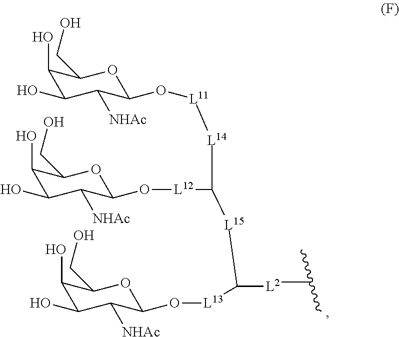

wherein:
each L¹¹, L¹², L¹³, L¹⁴, and L¹⁵ is an independently a linker;
L² is a linker;
[symbol] is an attachment point to the sense strand or the antisense strand.

In some embodiments, the ligand has a structure of:

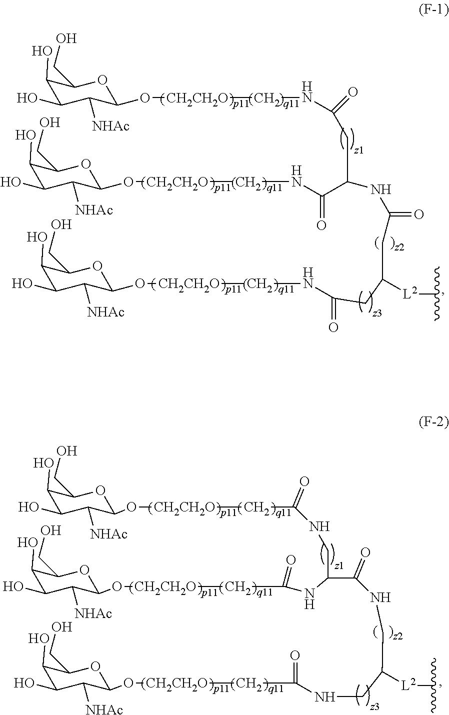

wherein:
each p11 and q11 is independently an integer from 0 to 12;
each z1, z2, and z3 is independently an integer of 0 to 12; and
[symbol] is an attachment point to the sense strand or the antisense strand.

In some embodiments, the ligand comprises the following structure:

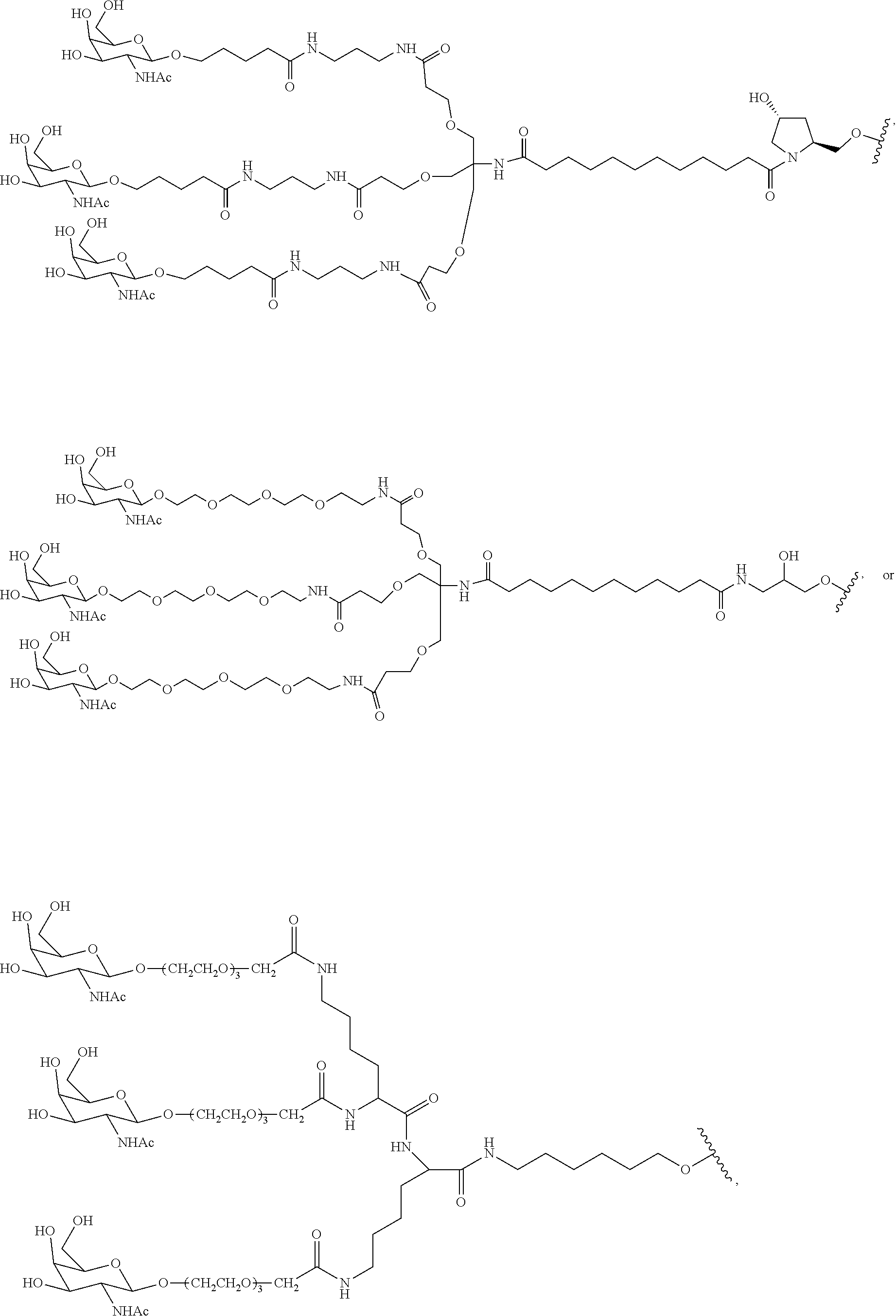

wherein
   [symbol] is an attachment point to the sense strand or the antisense strand.

In some embodiments, the ligand is conjugated to 3′ end of the sense strand to form the following structure:

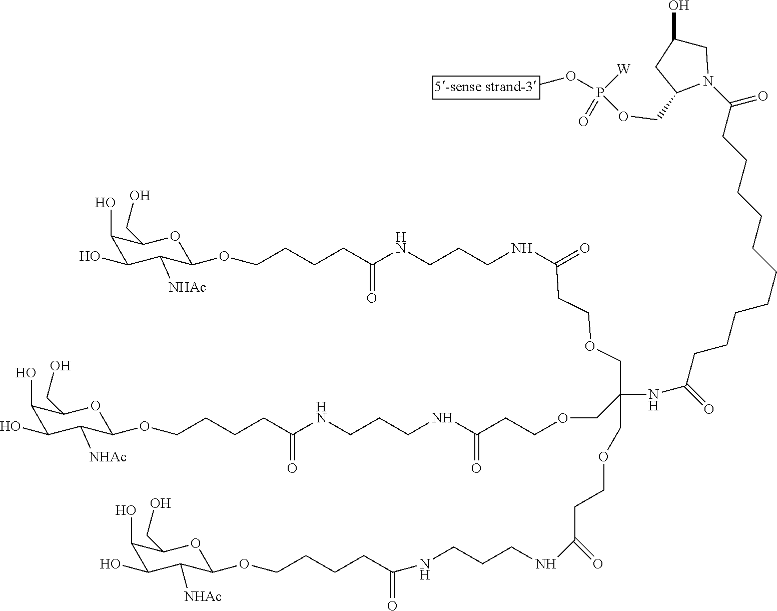

or a pharmaceutically acceptable salt,
   wherein W is —OH or —SH.

In some embodiments, the ligand is conjugated to 5′ end of the sense strand to form the following structure:

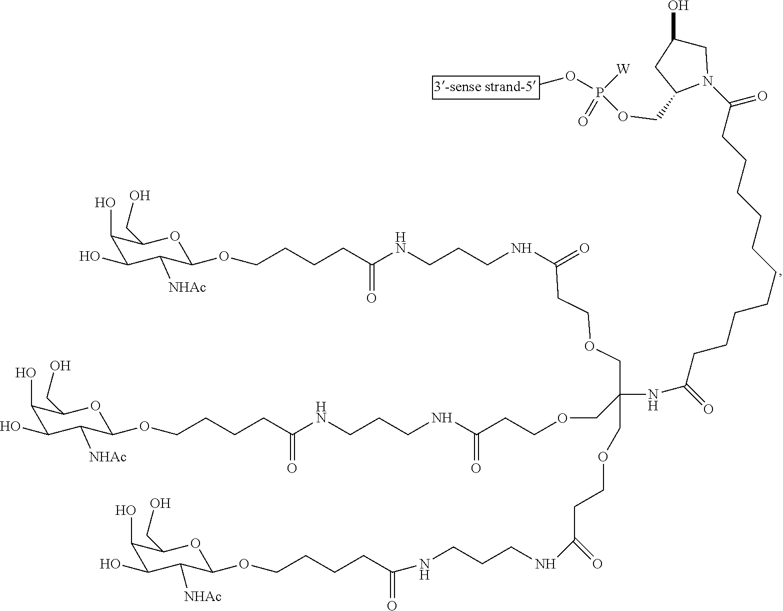

or a pharmaceutically acceptable salt,
   wherein W is —OH or —SH.

In some embodiments, W is —OH.

In some embodiments, the dsRNAi agent is in a pharmaceutically acceptable salt form.

In some embodiments, the pharmaceutically acceptable salt is a sodium salt.

In certain aspects, provided is a pharmaceutical composition comprising the dsRNAi agent as described herein, and a pharmaceutically acceptable carrier.

In some embodiments, the composition is in an aqueous solution form.

In some embodiments, the pharmaceutical composition further comprises an additional therapeutic agent selected from a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

In some embodiments, the additional therapeutic agent comprises the PCSK9 inhibitor.

In some embodiments, the PCSK9 inhibitor is a second dsRNAi agent.

In some embodiments, the second dsRNAi agent comprises inclisiran.

In certain aspects, provided is a combination of the dsRNAi agent as described herein and a second agent selected from a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an BAT inhibitor, a squalene synthase inhibitor, a monocyte chemoattractant protein (MCP)-I inhibitor, and a combination thereof.

In some embodiments, the second agent is a second dsRNAi agent.

In some embodiments, the second dsRNAi agent is a dsRNA agent that targets one or more of the genes selected from the group consisting of PCSK9, LPA, AGT, ACE, ACE2, AGTR1, AGTR2, ACAT, CETP, MTTP, PPAR, IBAT, FDFT1, ERG9, SQS1, Ccl2, CCR2, CCL7, CCL8. CCL13, and CCL16.

In some embodiments, the second dsRNAi agent comprises inclisiran.

In certain aspects, provided is a pharmaceutical composition comprising the combination as described herein.

In some embodiments, the second dsRNAi agent is in a pharmaceutically acceptable salt form.

In some embodiments, the pharmaceutically acceptable salt of the second dsRNAi agent is a sodium salt.

In some embodiments, the dsRNAi agent and the second agent are formulated in the same composition.

In some embodiments, the dsRNAi agent and the second agent are formulated in the separate compositions.

In certain aspects, provided is a method of inhibiting expression of 3-hydroxy-3-methylglutaryl-CoA reductase (HMGCR) in a subject comprising:
administering to the subject the dsRNAi agent as described herein or a pharmaceutically acceptable salt thereof, or the pharmaceutical composition as described herein.

In certain aspects, provided is method of lowering a level of low-density lipoprotein cholesterol (LDL-C) in a subject, comprising:
administering to the subject the dsRNAi agent as described herein or a pharmaceutically acceptable salt thereof, or the pharmaceutical composition as described herein.

In certain aspects, provided is a method of treating or preventing an HMGCR-associated disorder or disease in a subject, comprising:
administering to the subject the dsRNAi agent as described herein or a pharmaceutically acceptable salt thereof, or the pharmaceutical composition as described herein.

In some embodiments, the HMGCR-associated disorder or disease is hyperlipidemia, hypercholesterolemia, hypertriglyceridemia, mixed hyperlipidemia, primary hyperlipidemia, heterozygous familiar hypercholesterolemia (HeFH), homozygous familiar hypercholesterolemia (HoFH), congestive heart disease (CHD) or atherosclerosis.

In certain aspects, provided is a method of treating or preventing hyperlipidemia in a subject, comprising:
administering to the subject the dsRNAi agent as described herein or a pharmaceutically acceptable salt thereof, or the pharmaceutical composition as described herein.

In some embodiments, the hyperlipidemia is hypercholesterolemia, or hypertriglyceridemia.

In certain aspects, provided is a method of treating or preventing atherosclerotic cardiovascular disease (ASCVD) in a subject, comprising:
administering to the subject the dsRNAi agent as described herein or a pharmaceutically acceptable salt thereof, or the pharmaceutical composition as described herein.

In some embodiments, the dsRNAi agent or the pharmaceutical composition is administered subcutaneously or intravenously.

In some embodiments, the methods further comprises administering to the subject an additional therapeutic agent selected from a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

In some embodiments, the additional therapeutic agent is a second dsRNAi agent.

In some embodiments, the second dsRNAi agent comprises the PCSK9 inhibitor.

In some embodiments, the second dsRNAi agent comprises inclisiran.

In some embodiments, the dsRNAi agent or the pharmaceutical composition and the additional therapeutic agent are administered simultaneously.

In some embodiments, the dsRNAi agent or the pharmaceutical composition and the additional therapeutic agent are administered subsequently.

In some embodiments, the dsRNAi agent is administered before administering the additional therapeutic agent.

In some embodiments, the additional therapeutic agent is administered before administering the dsRNAi agent.

In some embodiments, the additional therapeutic agent is administered subcutaneously or intravenously.

In certain aspects, provided is a method of lowering a level of low-density lipoprotein cholesterol (LDL-C) in a subject, comprising:
administering to the subject the pharmaceutical composition as described herein.

In certain aspects, provided is a method of treating or preventing an HMGCR-associated disorder or disease in a subject, comprising:
administering to the subject the pharmaceutical composition as described herein.

In some embodiments, the HMGCR-associated disorder or disease is hyperlipidemia, hypercholesterolemia, hypertriglyceridemia, mixed hyperlipidemia, primary hyperlipidemia, heterozygous familiar hypercholesterolemia (HeFH), homozygous familiar hypercholesterolemia (HoFH), congestive heart disease (CHD) or atherosclerosis.

In certain aspects, provides is a method of treating or preventing hyperlipidemia in a subject, comprising:
administering to the subject the pharmaceutical composition as described herein.

In certain aspects, provided is a method of treating or preventing atherosclerotic cardiovascular disease (ASCVD) in a subject, comprising:
administering to the subject the pharmaceutical composition as described herein.

In some embodiments, the dsRNAi agent and the second agent is administered subcutaneously or intravenously.

In some embodiments, the dsRNAi agent and the second agent are administered simultaneously.

In some embodiments, the dsRNAi agent and the second agent are administered subsequently.

In some embodiments, the dsRNAi agent is administered before administering the second agent.

In some embodiments, the second agent is administered before administering the dsRNAi agent.

In some embodiments, in any of the methods described herein, the subject is a human.

In some embodiments, in any of the methods described herein, the subject has or is diagnosed with hyperlipidemia, hypercholesterolemia, hypertriglyceridemia, mixed hyperlipidemia, primary hyperlipidemia, heterozygous familiar hypercholesterolemia (HeFH), homozygous familiar hypercholesterolemia (HoFH), congestive heart disease (CHD) or atherosclerosis.

In some embodiments, in any of the methods described herein, the subject does not have a muscle side effect after the administrating the pharmaceutical composition of as described herein.

In certain aspects, provided is a method of reducing the risk of a major adverse cardiovascular event in a subject, comprising administering to the subject the dsRNAi agent as described herein or a pharmaceutically acceptable salt thereof, the pharmaceutical composition as described herein, the combination as described herein, or the pharmaceutical composition comprising the combination as described herein.

In some embodiments, the major adverse cardiovascular event is cardiovascular death, non-fatal myocardial infarction, non-fatal ischemic stroke, or urgent coronary revascularization.

In some embodiments, the subject has an established cardiovascular disease.

In some embodiments, the subject has not experienced a major atherosclerotic cardiovascular disease (ASCVD) event.

In certain aspects, provided is a kit comprising the dsRNAi agent as described herein or a pharmaceutically acceptable salt thereof, or the pharmaceutical composition as described herein.

In some embodiments, the kit further comprises an additional therapeutic agent selected from a proprotein convertase subtilisin kexin 9 (PCSK9) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

In some embodiments, the additional therapeutic agent is a second dsRNAi agent.

In some embodiments, the second dsRNAi agent is a dsRNA agent that targets one or more of the genes selected from the group consisting of PCSK9, LPA, AGT, ACE, ACE2, AGTR1, AGTR2, ACAT, CETP, MTTP, PPAR, IBAT, FDFT1, ERG9, SQS1, Ccl2, CCR2, CCL7, CCL8. CCL13, and CCL16.

In some embodiments, the second dsRNAi agent comprises the PCSK9 inhibitor.

In some embodiments, the second dsRNAi agent comprises inclisiran.

In some embodiments, the dsRNAi agent and the additional therapeutic agent are contained in a single vial.

In some embodiments, the dsRNAi agent and the additional therapeutic agent are contained in separate vials.

In certain aspects, provided is a kit comprising the pharmaceutical composition including the combination as described herein.

In some embodiments, the dsRNAi agent and the second agent are contained in a single vial.

In some embodiments, the dsRNAi agent and the second agent are contained in separate vials.

In some embodiments, the kir further comprises one or more applicators.

In some embodiments, the one or more applicators comprises a syringe.

Other aspects of the invention are disclosed infra.

### BRIEF DESCRIPTION OF DRAWINGS

FIGS. 1A to IC: Transcriptomic profiling with different lead sequences. The siRNA284, for example, which can be formed by SEQ ID NO: 284 and SEQ ID NO: 689, or modified variants thereof had HMGCR significant down-regulation as shown in FIG. 1A. The siRNA3, for example, which can be formed which can be formed by SEQ ID NO: 3 and SEQ ID NO: 408, or modified variants thereof had an exquisite off-target profile as shown in FIG. 1B. The siRNA6, for example, which can be formed by SEQ ID NO: 6 and SEQ ID NO: 411, or modified variants thereof (e.g., with or without GNA) had still some seed-mediated off-targeting profiling as shown in FIG. 1C.

FIG. 2: Compounds 1-4 as outlined in Table 5 of the disclosure are depicted and each modification (e.g., 2′ modified nucleosides and linkages) are depicted. “PO” means a chemical group to form a phosphate (phosphodiester) linkage and “PS” means linking group to form a phosphorothioate linkage, and “L96” refers to a ligand that is connected to the 3′-end of the sense strand via phosphate (phosphodiester) linkage. Figure discloses SEQ ID NOS 2606-2613, respectively, in order of appearance.

FIGS. 3A to 3B: Liver HMGCR mRNA concentrations for male C57BL/6 mice (n=4 per timepoint for each group) administered a single subcutaneous dose of either Compound 7 (gray) or Compound 6 (black) at 6 mg/kg are shown in FIG. 3A. Statistical significance was determined by ordinary one-way ANOVA and Sidak's multiple comparisons test. For Compound 7, statistically significant reductions in HMGCR mRNA abundance versus vehicle were observed at all post-dose timepoints, with the exception of day 28. A maximum mean reduction of 77±3% (P<0.001) occurred on day 21 and was, in large part, sustained through day 35 (−68±12%, P<0.01). Compound 6 also reduced liver HMGCR mRNA abundance in this model, with a maximum decrease of 79±12% at day 7. Incorporation of guide strand into liver RISC over time is illustrated in FIG. 3B. Statistical significance was determined by ordinary one-way ANOVA and Sidak's multiple comparisons test. In male C57BL/6 mice (n=4 per timepoint for each group) dosed with 6 mg/kg Compound 6 (black circles), a steep decline in RISC-loading was observed between days 7 and 21 post-dose. In contrast, RISC-loading was sustained through day 21 in mice administered Compound 7 at 6 mg/kg (gray squares). Moreover, at day 42 post-dose, RISC-loading was 100-fold greater in the Compound 7 versus Compound 6 group.

FIGS. 4A to 4B: Liver HMGCR mRNA concentrations for male C57BL/6 mice (n=4 or 5 per group) administered a single subcutaneous dose of PBS (white), Compound 5 (white/black), Compound 7 (black) or Compound 8 (gray) at 3 mg/kg are shown in FIG. 4A. The siRNAs share a same nucleotide sequence but have different chemical modifications. Statistical significance was determined by ordinary one-way ANOVA and Sidak's multiple comparisons test. At day 35 post-dose, statistically significant reductions in hepatic HMGCR mRNA abundance versus vehicle (PBS) were observed in mice dosed with Compound 7 (−55%, P<0.05 vs. PBS) and Compound 8 (−70%, P<0.01 vs. PBS). In mice dosed with Compound 8, liver HMGCR mRNA levels were significantly reduced vs. vehicle through day 56 post-dose (−51%, P<0.05). These results show durable HMGCR knockdown in mice, with Compound 8 performing better than Compound 7 in this experiment. Incorporation of guide strand into liver RISC over time is illustrated in FIG. 4B. Male C57BL/6 mice (n=4 or 5 per group) were administered a single subcutaneous dose of PBS (white) or Compound 5 (white/black), Compound 7 (black) or Compound 8 (gray) at 3 mg/kg. Statistical significance was determined by ordinary one-way ANOVA and Sidak's multiple comparisons test. Consistent with the liver HMGCR mRNA results, Compound 8 showed the highest RISC-loading at both timepoints evaluated. The difference observed between Compound 5 and Compound 8 at day 35 was robust and statistically significant (P<0.0001). These results show durable RISC-incorporation (through day 56 post-dose) for Compound 8 in mice.

FIGS. 5A to 5B: Nine- to ten-week old male Wistar Han rats received a single subcutaneous dose of vehicle (0.9% sodium chloride for injection, USP) or HMGCR GalNAc-conjugated siRNA (Compound 9) on study day 1. Livers for the measurement of HMGCR mRNA abundance were collected at necropsy (30 days post-dose). HMGCR mRNA levels are normalized to TATA-box binding protein (TBP) and represent mean±SD (n=5 per group) in FIG. 5A. Statistical significance was determined by ordinary one-way ANOVA and Dunnett's multiple comparisons test. Statistically significant reductions in hepatic HMGCR mRNA abundance versus vehicle were observed for all siRNA groups. Maximum target knockdown was achieved with doses of >30 mg/kg. Nine- to ten-week old male Wistar Han rats received a single subcutaneous dose of vehicle (0.9% sodium chloride for injection, USP) or HMGCR GalNAc-conjugated siRNA (Compound 9) on study day 1. Right bicep femoris skeletal muscle samples for the measurement of HMGCR mRNA abundance were collected at necropsy (30 days post-dose). Mean HMGCR mRNA abundance for vehicle and GalNAc-conjugated HMGCR siRNA (Compound 9) groups are shown in FIG. 5B and represent mean±SD (n=5 per group). Relative to vehicle, skeletal muscle HMGCR mRNA levels were minimally increased in all dose groups, with no dose-relatedness evident. None of these differences achieved statistical significance, when compared to the vehicle group. These findings demonstrate that, at doses up to 300 mg/kg, target knockdown is not observed in skeletal muscle 30 days post-dose.

FIGS. 6A to 6B: Low density lipoprotein receptor (LDLR) protein levels in a human liver cell line (Huh7) treated with HMGCR siRNA (Compound 1) or atorvastatin at a concentration of 25 nM are provided in FIG. 6A. Briefly, Huh7 cells were transfected with test or control siRNA using lipofectamine and incubated at 37° C. for 6 hours. After changing the culture media, cells were incubated for another 24 hours prior to processing for the measurement of mRNA abundance or LDLR protein levels. The same incubation times were used for cells treated with atorvastatin. Atorvastatin (25 μM in DMSO) was diluted in culture medium to achieve a concentration of 25 nM. HMGCR mRNA was reduced by 61% versus control in Huh7 cells treated with HMGCR siRNA (Compound 1). Total LDLR protein levels, as determined by Western blotting, increased by 57% or 20% in cells treated with HMGCR siRNA (Compound 1) or atorvastatin, respectively. These results demonstrate that, like a statin, an HMGCR siRNA (Compound 1) increases LDLR protein in a human liver cell line. LDLR protein levels in primary human hepatocytes (PHH) treated with HMGCR siRNA (Compound 1) or atorvastatin at a concentration of 10 nM are provided in FIG. 6B. Briefly, PHH were transfected with test or control siRNA using lipofectamine and incubated at 37° C. for 24 hours prior to processing for the measurement of mRNA abundance or LDLR protein levels. The same incubation times were used for cells treated with atorvastatin. Atorvastatin (25 μM in DMSO) was diluted in culture medium to achieve a concentration of 10 nM. HMGCR mRNA was reduced by 65% versus control in PHH cells treated with HMGCR siRNA (Compound 1). Total LDLR protein levels, as determined by Western blotting, increased by 48% or 80% in cells treated with HMGCR siRNA (Compound 1) or atorvastatin, respectively. These results demonstrate that, like a statin, an HMGCR siRNA (Compound 1) increases LDLR protein in primary human hepatocytes.

FIGS. 7A-7B: HMGCR protein levels in a human liver cell line (Huh7) treated with Compound 1 at a concentration of 12.5 or 25 nM are provided in FIG. 7A. Briefly, Huh7 cells were transfected with test or control siRNA using lipofectamine and incubated at 37° C. for 6 hours. After changing the culture media, cells were incubated for another 24 hours prior to processing for the measurement of HMGCR protein levels by Western blotting. Relative to the control siRNA, Compound 1 reduced HMGCR protein content by 51% and 73% at concentrations of 12.5 and 25 nM, respectively. These results demonstrate that Compound 1 significantly, and dose-dependently, reduces HMGCR protein content in a human liver cell line. HMGCR protein levels in a human liver cell line (Huh7) treated with atorvastatin at a concentration of 12.5 or 25 nM are provided in FIG. 7B. Atorvastatin (25 μM in DMSO) was diluted in culture medium to achieve the desired concentrations. Huh7 cells were incubated with atorvastatin or DMSO at 37° C. for 6 hours. After changing the culture media, cells were incubated for another 24 hours prior to processing for the measurement of HMGCR protein levels by Western blotting. Relative to DMSO, atorvastatin markedly augmented HMGCR protein levels, with 96% and 123% increases observed relative to control at concentrations of 12.5 and 25 nM, respectively. These results demonstrate that, in contrast to Compound 1, atorvastatin robustly increases HMGCR protein expression in a human liver cell line.

FIG. 8A-8B: Effects of Compound 1 on plasma total cholesterol concentrations in mice with humanized livers. Mean percent change ±SD in total cholesterol level versus baseline for the PBS and Compound 1 groups are shown in FIG. 8A, while results for individual animals in the Compound 1 group are presented in panel FIG. 8B. TC, total cholesterol.

FIGS. 9A-9B: Hepatic HMGCR mRNA abundance in male cynomolgus monkeys at pre-dose and days 28 and 85 after subcutaneous injection of two doses of Compound 1 given 34 days apart. A total of 3 liver biopsy samples were obtained from each animal during the study: one pre-dose (day 12) and two post-dose (days 28 and 85 after the first dose) to enable the measurement of hepatic HMGCR mRNA expression by RT-qPCR. HMGCR mRNA levels are normalized to TATA-box binding protein (TBP) and represent mean±SD (n=4 per group). Shapes indicate values for individual animals in each group. Statistical significance was determined by paired t-test. Mean hepatic HMGCR mRNA abundance was reduced versus pre-dose in all groups at both day 28 and 85; however, only the Compound 1, 5 mg/kg group, showed statistically significant reductions in target knockdown at both timepoints. *P<0.0003, **P<0.05 for the comparison with pre-dose values.

FIG. 10: Serum HDL cholesterol concentrations in male cynomolgus monkeys at post-dose timepoints through day 85 after subcutaneous injection of two doses of Compound 1 given 34 days apart. On days 1 and 35, male cynomolgus monkeys (n=4 per group) received Compound 1 at 5 or 10 mg/kg via subcutaneous injection. Blood samples were collected at 3 pre-dose timepoints and at multiple post-dose timepoints for the determination of serum HDL cholesterol levels, using a Cobas 8000 chemistry analyzer. Data represent mean±SD percent change versus pre-dose (average of measurements conducted on samples from 3 pre-dose blood collections) through day 85 (day of final liver biopsy) post initial dose.

FIG. 11: Dose response curve in Hep3B cells transfected with nine siRNAs (S1-S9) of Example 10 in an 8-point dilution series starting at 40 nM with a dilution factor of 1:6. % remaining HMGCR mRNA levels are displayed.

FIG. 12: Dose response curve in Hepa-1-6 cells transfected with nine siRNAs (S1-S9) of Example 10 and with reporter plasmid TR030 in a 7-point dilution series starting at 6.7 nM with a dilution factor of 1:6. % remaining luciferase signal was normalized to renilla signal.

FIG. 13: Dose response curve in Hepa-1-6 cells transfected with nine siRNAs (S1-S9) in Example 10 and with reporter plasmid TR029 in a 7-point dilution series starting at 6.7 nM with a dilution factor of 1:6. % remaining luciferase signal was normalized to renilla signal.

FIGS. 14A-14B: Liver HMGCR mRNA concentrations for male C57BL/6 mice (n=3 or 4 per group) administered a single subcutaneous dose of PBS (white), No1 and No2 siRNAs at 3 mg/kg are shown in FIG. 14A. No1 and No2 siRNAs share a same nucleotide sequence but have different chemical modifications. These results show durable HMGCR knockdown in mice, with No1 siRNA (with 2′-MOE clamp in the sense strand) performing better than No5 siRNA (without 2′-MOE clamp in the sense strand) at both timepoints evaluated (10 days or 42 days). Incorporation of guide strand into liver RISC over time is illustrated in FIG. 14B. Male C57BL/6 mice (n=3 or 4 per group) were administered a single subcutaneous dose of PBS, No1 siRNA, or No5 siRNA at 3 mg/kg. Statistical significance was determined by ordinary one-way ANOVA and Sidak's multiple comparisons test. Consistent with the liver HMGCR mRNA results, No1 siRNA showed the higher RISC-loading than No5 siRNA at both timepoints evaluated (10 days or 42 days).

FIGS. 15A-15B: Liver HMGCR mRNA concentrations for male C57BL/6 mice (n=3 or 4 per group) administered a single subcutaneous dose of PBS (white), No1 to No4 siRNAs at 3 mg/kg are shown in FIG. 15A. No1 to No4 siRNAs share a same nucleotide sequence but have different chemical modifications. These results show durable HMGCR knockdown in mice, and No1 and No2 (with MOE clamp and six or eight 3′-PS in the sense strand) performed similarly as No3 and No4 (with TNA clamp and six or eight 3′-PS in the sense strand) in this experiment. Incorporation of guide strand into liver RISC over time is illustrated in FIG. 15B. Male C57BL/6 mice (n=3 or 4 per group) were administered a single subcutaneous dose of PBS, No1 to No4 siRNA at 3 mg/kg. Statistical significance was determined by ordinary one-way ANOVA and Sidak's multiple comparisons test. Consistent with the liver HMGCR mRNA results, No1 to No4 siRNAs showed similar RISC-loading at 42 days.

FIG. 16: Luciferase reporter assay results with Compounds 11-13 (Example 12) targeting HMGCR mRNA at position 115 in comparison to Compound 10 (targeting HMGCR mRNA at position 126).

FIG. 17: Luciferase reporter assay results with Compounds 14-16 (Example 12) targeting HMGCR mRNA at position 2835 in comparison to Compound 10 (targeting HMGCR mRNA at position 126).

FIG. 18: Percent reduction in plasma LDL-C concentrations in cynomolgus monkeys administered inclisiran and an HMGCR siRNA in Table 4 compared to the plasma LDL-C concentration administered inclisiran alone (inclisiran+PBS).

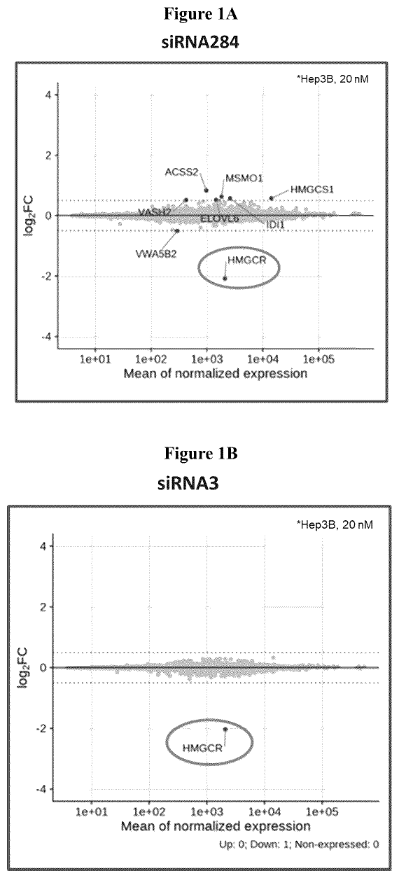

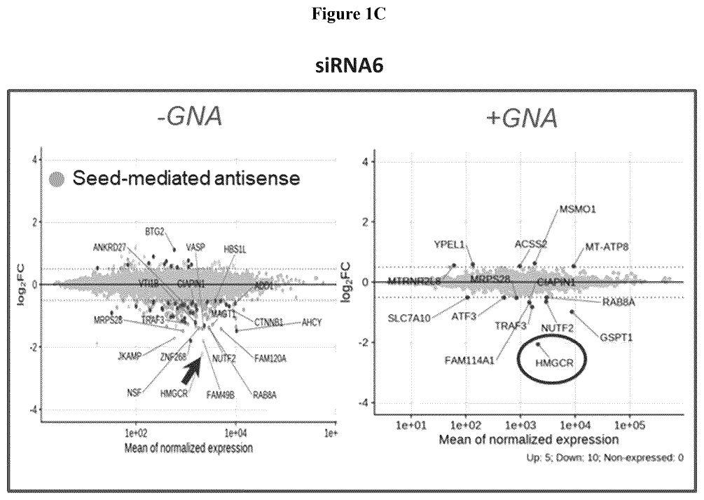

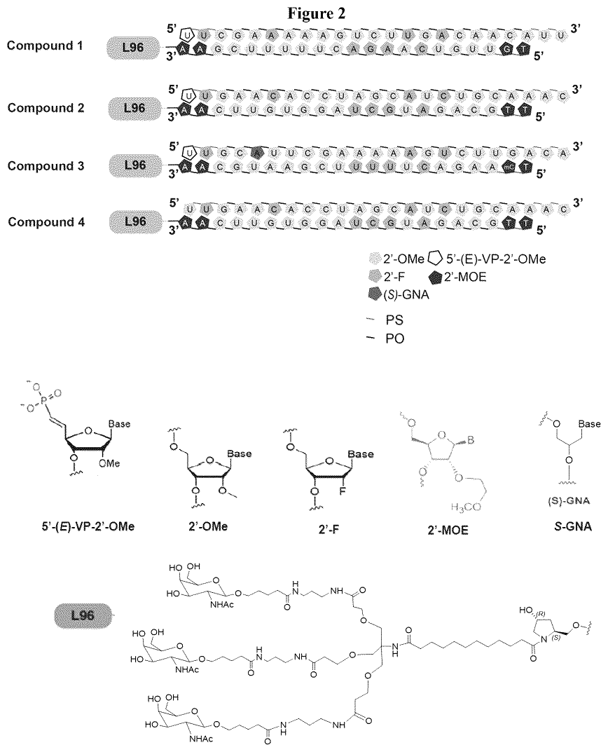

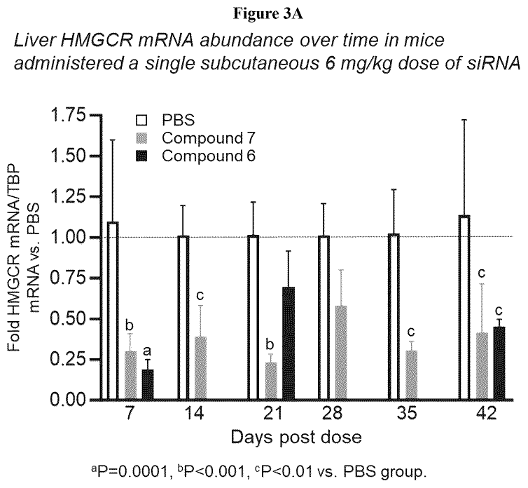

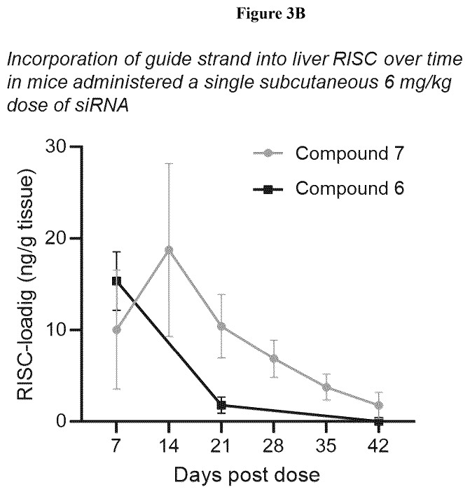

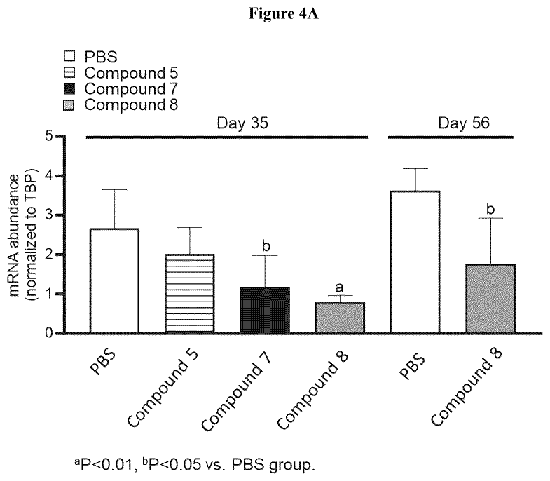

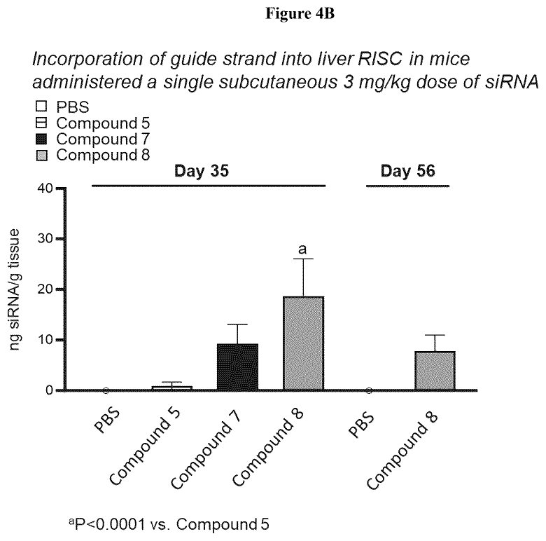

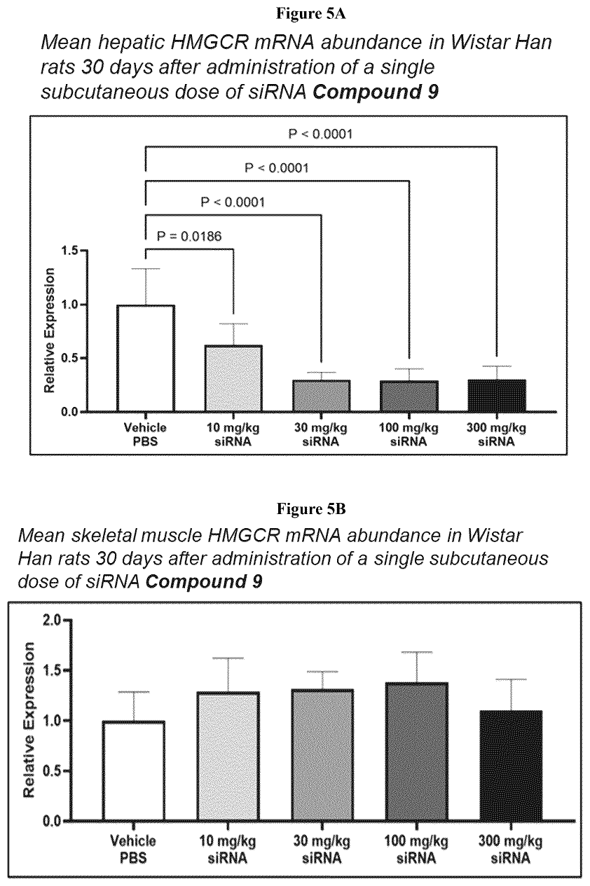

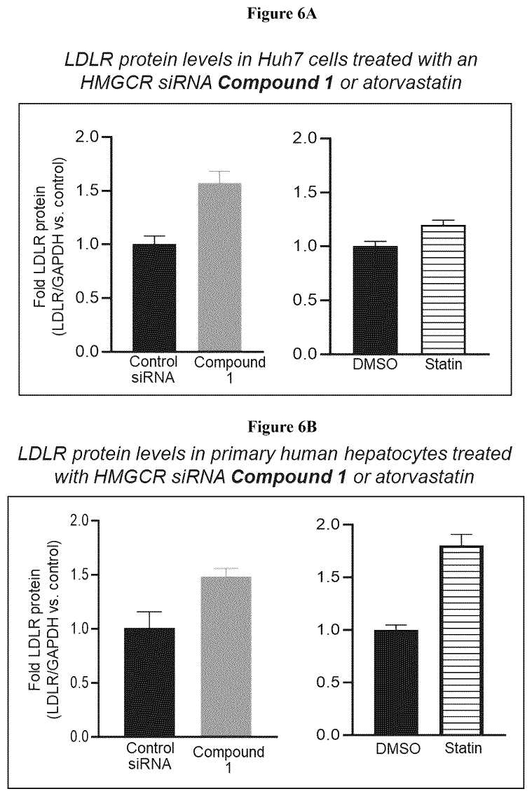

### DETAILED DESCRIPTION

### Definitions

Unless defined otherwise, all technical terms, scientific terms, abbreviations, chemical structures, and chemical formulae used herein have the same meaning as is commonly understood by one of ordinary skill in the art. The chemical structures and formulae set forth herein are constructed according to the standard rules of chemical valency known in the chemical arts. All patents, applications, published applications, and other publications referenced herein are incorporated by reference in their entirety unless stated otherwise.

All patents, applications, published applications, and other publications referenced herein are incorporated by reference in their entirety unless stated otherwise. Unless otherwise indicated, conventional methods of mass spectroscopy, NMR, HPLC, protein chemistry, biochemistry, recombinant DNA techniques, and pharmacology are employed.

Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components. As used herein, the term “a,” “an,” “the” and similar terms used in the context of the present invention (especially in the context of the claims) are to be construed to cover both the singular and plural unless otherwise indicated herein or clearly contradicted by the context.

Unless otherwise indicated, all numbers, values, and/or expressions referring to nucleotide lengths, inhibition, activities, dosages, contents, and formulations used herein are to be understood as modified in all instances by the term “about” as such numbers are inherently approximations that are reflective of, among other things, the various uncertainties of measurement encountered in obtaining such values. Further, unless specifically stated or obvious from context, as used herein, the term “about” is understood as within a range of normal tolerance in the art, for example within 2 standard deviations of the “mean. “About” may be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The term “nucleic acid” means a compound containing at least two nucleotide monomers covalently linked together. Nucleic acids include polynucleotides and oligonucleotides, including double-stranded oligonucleotides and single-stranded oligonucleotides, and modified versions thereof.

The term “nucleotide” means a compound including a nucleoside and a phosphate group (or interchangeably, phosphodiester linkage) that are covalently attached at 5′ position or 3′ position of the pentofuranosyl sugar (e.g., ribose or deoxyribose). In certain aspects, the nucleotide is a ribonucleotide (RNA) having the ribose as the pentofuranosyl sugar. In certain aspects, a nucleotide is a deoxyribonucleotide (DNA) having the deoxyribose (2′-deoxyribose) as the pentofuranosyl sugar. Unless otherwise specifically indicated, when referring a “nucleotide” in a chain of nucleotides (e.g., oligonucleotides), e.g., X₁ to X₂₁ and X₁ to X_{23′}, a nucleotide is meant by a nucleoside and a phosphate group (or phosphodiester linkage) that is covalently attached at 3′ position of the pentofuranosyl sugar (e.g., ribose or deoxyribose).

The term “nucleoside” means a monomer consisting of a nucleobase and a pentofuranosyl sugar (e.g., ribose or deoxyribose). A nucleoside including a ribose sugar ring has to a structure of

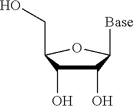

or a pharmaceutically acceptable salt thereof, and a nucleotide including a deoxyribose sugar ring has a structure

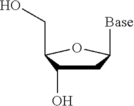

or a pharmaceutically acceptable salt, wherein in each structure, “Base” is a nucleobase.

The term “nucleobase” or “base,” as used herein, means the heterocyclic base moiety of a nucleoside or nucleotide. Non-limiting examples of nucleobases includes cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g., hypoxanthine analogue), xanthine or a derivative thereof (e.g., xanthine analogue), 7-methylguanine or a derivative thereof (e.g., 7-methylguanine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In some embodiments, the nucleobase is adenine, guanine, hypoxanthine, xanthine, theobromine, caffeine, uric acid, or isoguanine, which may be optionally substituted or modified. In some embodiments, the nucleobase is

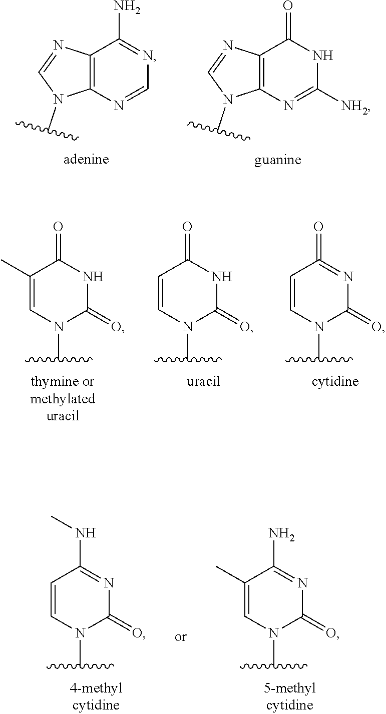

which may be optionally substituted or modified, wherein “ [symbol] ” denotes the point of attachment to a pentofuranosyl sugar ring (e.g., 1′ position).

The term “phosphate,” or “phosphate group” as used herein a chemical species made of one phosphorus atom and four oxygen atoms

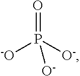

or esters, salts, or acids thereof. In certain aspects, when the phosphate groups are positioned between adjacent nucleosides in RNA or DNA strand and form a “backbone” of the oligonucleotides, these terms “phosphate,” or “phosphate group” may be interchangeable used as “phosphate group,” “phosphate linkage,” “phosphodiester linkage,” or “linkage.” For example, the phosphate or phosphodiester linkage in the backbone of RNA or DNA may have the structures of

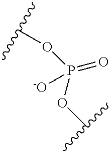

or esters, salts (e.g., pharmaceutically acceptable salts), or acids (e.g.,

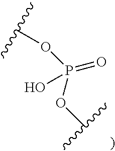 [SMILES: *OP(=O)(O)O*]

thereof, wherein “ [symbol] ” denotes the point of attachment to pentofuranosyl sugar rings (e.g., 5′ and 3′ positions) in adjacent nucleosides. In certain aspects, a variant of a phosphate or phosphodiester linkage, e.g., phosphorothioate (PS) linkage, can replace a phosphate group (or phosphodiester linkage) in the backbone and connect two adjacent nucleosides. In certain aspects, a variant of a phosphate or phosphodiester linkage, e.g., phosphorothioate (PS) linkage or vinylphosphonate (VP) group, may be additionally attached at 3′ end or 5′ end of the oligonucleotides (e.g., RNA or DNA), e.g., 3′-OH or 5′-OH position of the terminal pentofuranosyl sugar (e.g., ribose or deoxyribose), so as to act as chemically or biologically functional group. In certain aspects, a variant of phosphate or phosphodiester linkage may also be referred as a phosphorus-derived internucleoside linkage that includes at least one phosphorus atom in the backbone.

Unless otherwise indicated herein, an unmodified RNA (or “ribonucleotide”) in a chain of nucleotides (e.g., mRNA, rRNA, or sense strand or antisense strand of siRNA) as disclosed refers to a structure of

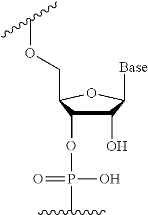

or a pharmaceutically acceptable salt thereof. Likewise, an unmodified DNA (or “deoxyribonucleotides”) in a chain of nucleotides (e.g., genomic DNA or cDNA) as disclosed herein specifically refers to a structure of

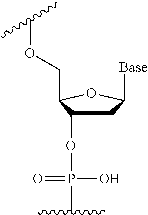

or a pharmaceutically acceptable salt thereof. In each structure “Base” is a nucleobase and [symbol] is an attachment point to the adjacent nucleotides.

Unless otherwise indicated herein, when an unmodified RNA is the first nucleotide from the 5′ end of an RNA chain (e.g., mRNA or sense strand or antisense strand of siRNA), that nucleotide has a structure of

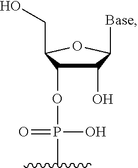

or a pharmaceutically acceptable salt thereof. Likewise, when an unmodified DNA is the first nucleotide from the 5′ end of a DNA chain (e.g., genomic DNA or cDNA), that nucleotide has a structure of

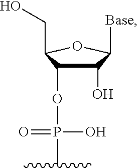

or a pharmaceutically acceptable salt thereof. In each structure “Base” is a nucleobase and [symbol] is an attachment point (5′ oxygen) to the adjacent nucleotides.

Alternatively, for example, the first nucleotide from the 5′ end of an RNA chain (e.g., mRNA, or sense strand or antisense strand of siRNA), that nucleotide has a structure of

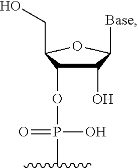

or a pharmaceutically acceptable salt thereof and the first nucleotide from the 5′ end of a DNA chain (e.g., genomic DNA or cDNA), that nucleotide has a structure of

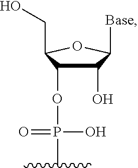

or a pharmaceutically acceptable salt thereof, when [symbol] is an attachment point (5′ oxygen) to the adjacent nucleotides.

Unless otherwise indicated herein, when an unmodified RNA is the first nucleotide from the 3′ end of an RNA chain (e.g., mRNA, or sense strand or antisense strand of siRNA), that nucleotide has a structure of

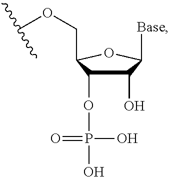

or a pharmaceutically acceptable salt. Likewise, when an unmodified DNA is the first nucleotide from the 3′ end of a DNA chain (e.g., genomic DNA or cDNA), that nucleotide has a structure of

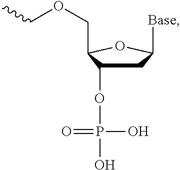

or a pharmaceutically acceptable salt thereof. In certain embodiments, when an unmodified RNA is the first nucleotide from the 3′ end of an RNA chain (e.g., mRNA, or sense strand or antisense strand of siRNA) that nucleotide does not include 3′ end phosphate group or phosphodiester linkage, for example, which has been removed during hydrolysis or synthesis, has a structure of

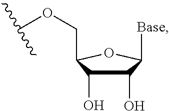

or a pharmaceutically acceptable salt. Likewise, when an unmodified DNA is the first nucleotide from the 3′ end of a DNA chain (e.g., genomic DNA or cDNA), that nucleotide does not include 3′ end phosphate group, for example, which has been removed during hydrolysis or synthesis, has a structure of

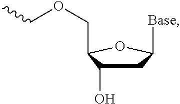

or a pharmaceutically acceptable salt thereof. In each structure “Base” is a nucleobase and [symbol] is an attachment point (e.g., phosphorus of the phosphate linkage) to the adjacent nucleotides.

[symbol]

A code “A”, “G”, “C”, or “U” presented in a sequence list as disclosed herein stand for a RNA nucleotide that contains adenine, guanine, cytosine, or uracil as a base, respectively. A code “dA”, “dG”, “dC” or “dT” presented in a sequence list as disclosed herein stand for a DNA nucleotide that contains adenine, guanine, cytosine, and thymine as a base, respectively. In some embodiments, the code “T” may be present in a RNA sequence then it may refer to a nucleotide (e.g. modified nucleotide) that thymine as a base.

The term “oligonucleotide” means a shorter length nucleic acid, e.g. of less than 100 nucleotides in length. Oligonucleotides may be single-stranded or double-stranded. In some embodiments, an oligonucleotide may include naturally occurring ribonucleotides, naturally occurring deoxyribonucleotides, and/or nucleotides having one or more modifications to a naturally occurring terminus, sugar, nucleobase, and/or internucleoside linkage. Non-limiting examples of oligonucleotides include double-stranded oligonucleotides (e.g., dsRNA), single-stranded oligonucleotides (e.g., single stranded RNA or ssRNA), antisense oligonucleotides (“ASO”), small interfering RNA (siRNA), microRNA mimics, short hairpin RNAs (shRNA), single-strand small interfering RNA (ssRNAi), RNaseH oligonucleotides, anti-microRNA oligonucleotides, steric blocking oligonucleotides, exon-skipping oligonucleotides, CRISPR guide RNAs, and aptamers. In certain aspects, the oligonucleotide is a dsRNA and each strand has a length less than 100 nucleotides (“nt”), less than 90 nt, less than 80 nt, less than 70 nt, less than 60 nt, less than 50 nt, less than 40 nt, less than 35 nt, less than 30 nt, less than 28 nt, less than 26 nt, less than 25 nt, less than 24 nt, less than 23 nt, less than 22 nt, less than 21 nt, less than 20 nt, less than 19 nt, less than 18 nt, less than 17 nt, less than 16 nt, or 15 nt.

The terms “iRNA”, “RNAi agent,” “iRNA agent,”, “RNA interference agent” as used interchangeably herein, refer to an agent that contains RNA as that term is defined herein, and which mediates the targeted cleavage of an RNA transcript (mRNA) via an RNA-induced silencing complex (RISC) pathway. An RNAi agent directs the sequence-specific degradation of mRNA through a process and thereafter inhibits expression of the gene encoded by the mRNA in a cell in vivo, e.g., in a subject (e.g., any vertebrate, mammal, or human).

The term “small interfering RNA” or “siRNA” means a double-stranded oligonucleotide (dsRNA) formed with two anti-parallel, and partially, substantially or fully complementary nucleic acid strands (e.g., a first strand and a second strand; or a “sense” strand and an “antisense” strand), which interferes with the expression of genes in a sequence-specific manner by facilitating mRNA degradation before translation through the RNA interference pathway. In some embodiments, depending on the context, the first strand can be a “guide” or antisense strand, and the second strand can be a “passenger” or sense strand. In some embodiments, depending on the context, the “first” strand can be a passenger or sense strand, and the “second” strand can be a guide or antisense. In certain aspects, an “RNAi agent” or “siRNA agent,” as used herein, refers a double-stranded RNA (dsRNA) with or without a ligand or other conjugate, and may be interchangeably used with a term “double stranded RNAi agent (dsRNAi agent),” or “dsRNA agent.” In certain aspect of the disclosure, the term “siRNA” can be used to describe a dsRNA with specific nucleotide sequences (unmodified or modified nucleotide sequences), without a ligand or other conjugate.

The term “antisense strand,” as used herein, refers an oligonucleotide (e.g., RNA) of an siRNA or a dsRNAi that is complementary (e.g., partially, substantially, or fully complementary) to the target mRNA and is incorporated into the RNA-induced silencing complex (RISC) to direct gene silencing in a sequence-specific manner through the RNA interference pathway. An antisense strand may also be referred to as the “guide strand.” In some embodiments, the antisense strand may have a length from 15-30 nt, 15-26 nt, 15-23 nt, 15-22 nt, 15-21 nt, 15-20 nt, 15-19 nt, 15-18 nt, 15-17 nt, 18-30 nt, 18-26 nt, 18-23 nt, 18-22 nt, 18-21 nt, 18-20 nt, 19-30 nt, 19-26 nt, 19-23 nt, 19-22 nt, 19-21 nt, 19-20 nt, 19 nt, 20-30 nt, 20-26 nt, 20-25 nt, 20-24 nt, 20-23 nt, 20-22 nt, 20-21 nt, 20 nt, 21-30 nt, 21-26 nt, 21-25 nt, 21-24 nt, 21-23 nt, 21-22 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, or 36 nt.

The term “sense strand,” as used herein, refers an oligonucleotide that is complementary (e.g., partially, substantially, or fully complementary) to the antisense strand. The sense strand is typically degraded following incorporation of the antisense strand into RISC. The sense strand may also be referred to as the “passenger strand.” In some embodiments, the sense strand may have a length from 15-30 nt, 15-26 nt, 15-23 nt, 15-22 nt, 15-21 nt, 15-20 nt, 15-19 nt, 15-18 nt, 15-17 nt, 18-30 nt, 18-26 nt, 18-23 nt, 18-22 nt, 18-21 nt, 18-20 nt, 19-30 nt, 19-26 nt, 19-23 nt, 19-22 nt, 19-21 nt, 19-20 nt, 19 nt, 20-30 nt, 20-26 nt, 20-25 nt, 20-24 nt, 20-23 nt, 20-22 nt, 20-21 nt, 20 nt, 21-30 nt, 21-26 nt, 21-25 nt, 21-24 nt, 21-23 nt, 21-22 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, or 36 nt.

The term “complementary” means that a nucleotide (e.g., RNA or DNA) or a sequence of nucleotides are capable of base pairing non-covalently via hydrogen bonding with another nucleotide or sequence of nucleotides. As described herein and commonly known in the art the complementary (matching) nucleotide of adenosine is thymidine or uridine and the complementary (matching) nucleotide of guanosine is cytidine. The complementarity of sequences may be partial, in which only some of the nucleic acids match according to base pairing, or complete, where all the nucleic acids match according to base pairing. For example, two sequences that are complementary to each other, may have a specified percentage of nucleotides that participate in nucleobase-pairing (i.e., about 50% complementarity, preferably 50%, 55%, 65%, 70%, 75%, 80%, 85%, 90%, 91%, 92%, 93%, 94%, 95%, 96%, 97%, 98%, 99%, or greater complementarity over a specified region). In some embodiments, two sequences are partially complementary when the percentage of nucleotides that participate in nucleobase-pairing is about 50%, about 55%, about 65%, about 70%, about 75%, or about 80%, or ranges from about 50% to about 80%. In some embodiments, two sequences are substantially complementary when the percentage of nucleotides that participate in nucleobase-pairing is about 80%, about 81%, about 82%, about 83%, about 84%, about 85%, about 86%, about 87%, about 88%, about 89%, about 90%, about 91%, about 92%, about 92%, about 93%, about 94%, or about 95%, or ranges from about 80% to about 95%.

Examples of complementary (e.g., partially, substantially, or fully complementary) sequences are sense and antisense sequences, wherein the sense sequence contains complementary (e.g., partially, substantially, or fully complementary) nucleotides to the antisense sequence and thus forms the complement of the antisense sequence. In certain aspects, a sense strand and an antisense strand of a double-stranded oligonucleotide (e.g., double stranded RNA) are substantially or fully complementary over their entire lengths. In some embodiments, a sense strand and an antisense strand of dsRNA are substantially or fully complementary over the entire length of the double-stranded region of the siRNA, and one or both termini of either strand comprises single-stranded nucleotides.

Another examples of complementary (e.g., partially, substantially, or fully complementary) sequences are an antisense strand and its target mRNA sequence. In certain aspects, an antisense strand is substantially or fully complementary to its target mRNA. For example, the complementary (e.g., partially, substantially, or fully complementary) sequences may be between an antisense strand and a coding region of the target mRNA, or a non-coding sequence of the target mRNA. In certain aspects, an antisense strand is substantially, or fully complementary to its target mRNA to reduce or eliminate off-target profile for and to improve down-regulation of the target gene (e.g., gene of the target mRNA sequence).

The terms “identical” or percent “identity,” in the context of two or more nucleic acids or polypeptide sequences, refer to two or more sequences or subsequences that are the same or have a specified percentage of amino acid residues or nucleotides that are the same (i.e., at least 60% identity, or at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or within a range defined by any of two of the preceding values, identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). This definition also refers to, or may be applied to, the complement of a test sequence. The definition also includes sequences that have deletions and/or additions, as well as those that have substitutions. As described below, the preferred algorithms can account for gaps, insertions and the like. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

As used herein, “target sequence” or “target gene” refer to a contiguous portion of the nucleotide sequence of an mRNA molecule formed during the transcription of a gene including mRNA that is a product of RNA processing of a primary transcription product. The target portion of the sequence will be at least long enough to serve as a substrate for RNAi-directed cleavage at or near that portion. For example, the target sequence will generally be from 9-36 nucleotides (“nt”) in length, e.g., 15-30 nt in length, including all sub-ranges therebetween. As non-limiting examples, the target sequence may have a length from 15-30 nt, 15-26 nt, 15-23 nt, 15-22 nt, 15-21 nt, 15-20 nt, 15-19 nt, 15-18 nt, 15-17 nt, 18-30 nt, 18-26 nt, 18-23 nt, 18-22 nt, 18-21 nt, 18-20 nt, 19-30 nt, 19-26 nt, 19-23 nt, 19-22 nt, 19-21 nt, 19-20 nt, 19 nt, 20-30 nt, 20-26 nt, 20-25 nt, 20-24 nt, 20-23 nt, 20-22 nt, 20-21 nt, 20 nt, 21-30 nt, 21-26 nt, 21-25 nt, 21-24 nt, 21-23 nt, 21-22 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, or 36 nt.

The term “ligand,” as used herein, refers to a compound or moiety that can impose characteristics to provide additional properties, e.g., affinity or cell delivery efficiency, to an RNAi (e.g., dsRNAi) as described herein. The ligand may be coupled or conjugated directly to the RNAi (e.g., sense strand or antisense strand of dsRNA), or indirectly to the RNAi agent (e.g., sense strand or antisense strand of dsRNA) via an intervening linker (“linker”). When a ligand is conjugated or coupled indirectly to the RNAi (e.g., dsRNA) via a linker, the ligand may be formed of a core moiety (e.g., targeting moiety) that has specific function to provide affinity or efficacy and the linker that provides merely an optimal distance, e.g., between the core moiety and the RNAi agent (dsRNA). In certain aspects, the term “ligand” embraces the ligand in combination with the linker. Examples of ligands or targeting moieties thereof may include, but not be limited to, one or more selected from a synthetic or natural compound, a peptide, an antibody, a carbohydrate (e.g., sugar moiety), or an additional nucleic acid.

The term “modified nucleotide” means a nucleotide having one or more modifications relative to a naturally occurring nucleotide, e.g., RNA. The modified nucleotide may be selected over an unmodified form because of desirable properties such as, for example, enhanced cellular uptake, enhanced affinity for other oligonucleotides or nucleic acid targets, increased stability in the presence of nucleases, and/or reduced immune stimulation. In certain aspects, the modification may be present in at least one of (i) an internucleoside linkage (“linkage”), (ii) a nucleobase, and (iii) a sugar moiety of the nucleotide. In certain aspects, the modification is present in the internucleoside linkage, e.g., by chemically modifying a phosphate (or phosphodiester) linkage or replacing a phosphate (or phosphodiester) linkage with other linking groups. In certain aspects, the modification is present in a sugar moiety, i.e., ribose ring, by substituting hydroxyl group on 2′ position of the ribose ring with other chemical group or by replacing a ring structure with other heterocycloalkyl or cycloalkyl, glycol group having a structure of

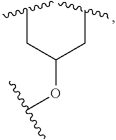 [SMILES: *CC(C*)O*]

bicyclic or bridged ring on the ribose such as locked nucleic acid (LNA) having a structure of

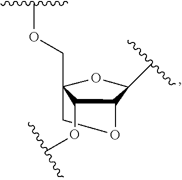 [SMILES: *OC[C@@]12COC([C@H](*)O1)[C@H]2O*]

or the like. In certain aspects, the modification is present in a nucleobase (e.g., A, G, C, T, or U) by chemical modification in a nucleobase by replacing the nucleobase with other moiety, for example, by replacing one naturally occurring nucleobase with another naturally occurring nucleobase. In certain aspects, a modified nucleotide may contain a modification in a sugar moiety and an unmodified phosphate (or phosphodiester) linkage. In certain aspects, a modified nucleotide may have a modification in a sugar moiety but with an unmodified nucleobase. In certain aspects, a modified nucleotide may have a modification in a sugar moiety and a nucleobase. In certain aspects, a modified nucleotide may have a modification in a sugar moiety and a phosphate (or phosphodiester) linkage. In certain aspects, a modified nucleotide may have a modification in a sugar moiety, a phosphate (or phosphodiester) linkage and a nucleobase. In certain aspects, a modified nucleotide may have an unmodified sugar moiety and an unmodified phosphate (or phosphodiester) linkage. In certain aspects, a modified nucleotide may have an unmodified sugar moiety and an unmodified nucleobase. In certain aspects, a modified nucleotide may have an unmodified sugar moiety and a modified nucleobase. In certain aspects, a modified nucleotide may have an unmodified sugar moiety and a modified phosphate (or phosphodiester) linkage. In certain aspects, a modified nucleotide may have a modified sugar moiety, a modified phosphate (or phosphodiester) linkage and a modified nucleobase.

The term “modified phosphate group,” or “modified phosphodiester linkage” as used herein refers to a chemical group in place of a phosphate group (or phosphodiester linkage) in a nucleotide as being attached to the 3′ end (3′ carbon) of the pentofuranosyl group.

The terms “identical” or percent “identity,” in the context of two or more nucleic acids or polypeptide sequences, refer to two or more sequences or subsequences that are the same or have a specified percentage of amino acid residues or nucleotides that are the same (i.e., at least 60% identity, or at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or within a range defined by any of two of the preceding values, identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). This definition also refers to, or may be applied to, the complement of a test sequence. The definition also includes sequences that have deletions and/or additions, as well as those that have substitutions. As described below, the preferred algorithms can account for gaps, insertions and the like. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

Throughout the disclosure, nucleotide positions or coordinates are relative to the beginning (5′ end) of the reference transcript.

The term “overhang” or “nucleotide overhang” herein refers to at least one unpaired nucleotide that protrudes from the end of at least one of the two strands of the duplex structure of an RNAi agent. In some embodiments, when a 3′-end of one strand extends beyond the 5′-end of the other strand, or vice versa, this forms a nucleotide overhang, e.g., the unpaired nucleotide(s) form the overhang.

“Blunt” or “blunt end” means that there are no unpaired nucleotides at that end of the double stranded RNAi agent, i.e., no nucleotide overhang. A “blunt ended” RNAi agent is a dsRNA that is double-stranded over its entire length, i.e., no nucleotide overhang at either end of the molecule.

A “mismatch” is defined herein as a difference between the base sequence (e.g., A instead of G) or length when two sequences are maximally aligned and compared. In certain aspects, the term “mismatch” means a nucleobase of a first oligonucleotide (e.g., a first strand) that is not capable of pairing with a nucleobase at a corresponding position of a second oligonucleotide (e.g., a second strand).

The term “non-end” herein refers to a position between the 3′ end and the 5′ end of the sense or antisense strand.

The term “3-hydroxy-3-methylglutaryl-CoA reductase,” as used herein and also interchangeably used with the term “HMGCR,” refers to a gene or protein (e.g., enzyme or reductase) thereof that converts 3-hydroxy-3-methylglutaryl coenzyme A (“HMG-CoA”) to mevalonate in cholesterol or isoprenoid synthesis. The term “HMGCR” also includes isoforms of proteins encoded by HMGCR mRNA sequences expressed in any vertebrate or mammals (e.g., human, mouse, rat, monkey, dog, cat, horse, pig, or cow).

In certain aspects, HMGCR may be identified with NCBI Gene ID, for example, human HMGCR Gene ID: 3156, mouse HMGCR Gene ID: 15357, rat HMGCR Gene ID: 25675, Rhesus monkey HMGCR Gene ID: 705479, dog HMGCR Gene ID: 479182, or cat HMGCR Gene ID: 101098922.

In certain aspects, HMGCR may be identified with mRNA transcript, for example, human HMGCR mRNA transcript (e.g., NM_000859.3; NM_001130996.2; and NM_001364187.1), mouse HMGCR mRNA transcript (e.g., NM_008255.2; NM_001360165.1; and NM_001360166.1), rat HMGCR mRNA transcript (e.g., NM_013134.2), Cynomolgus monkey HMGCR mRNA (e.g., XM_005557178.1), Rhesus monkey HMGCR mRNA (e.g., XM_001104607.4; and XM_002804417.3), dog HMGCR mRNA (e.g., XM_038471609.1; XM_038471611.1; and XM_038471610.1) and cat HMGCR mRNA (e.g., XM_003981075.6; and XM_019835641.3). In certain aspects, HMGCR may be identified with amino acid sequences, such as such as human HMGCR protein (e.g., NP_000850.1; NP_001124468.1; and NP_001351116.1), mouse HMGCR protein (e.g., NP_032281.2; NP_001347094.1; and NP_001347095.1), rat HMGCR protein (e.g., NP_037266.2), Rhesus monkey protein (e.g., XP_001104607.3; and XP_002804463.3), dog HMGCR protein (e.g., XP_038327537.1; XP_038327539.1; and XP_038327538.1), or cat HMGCR protein (e.g., XP_003981124.1 and XP_019691200.1). Examples of HMGCR gene, mRNA and proteins are not limited to the above list and may further include examples publicly available information from web database, for example, in GenBank, UniProt, Ensembl, Alliance and the like.

In certain aspects, HMGCR may include a fragment, variant, or mutant of the protein that may have the same or similar amino acid sequences (e.g., having about 80%, 85%, 90%, 95%, or 99% or greater of similarity or identity of amino acid sequences) with any one of the above listed HMGCR gene (mRNA) or protein sequences. In certain aspects, HMGCR may include a fragment, variant, or mutant of the protein having the same or in similar in vivo or in vitro enzymatic (e.g., reductase) activity, for example, having about 80%, 85%, 90%, 95%, or 99% or the native enzyme activity, to produce mevalonate from HMG-CoA.

The term “Compound” as used herein refers to a double stranded RNA (e.g., HMGCR dsRNA or dsRNAi agent) that is conjugated with a ligand or a delivery moiety, while a term “compound” denotation may refer to a substance, molecule or chemical entity that can be chemically defined and/or identifiable.

As defined herein, the term “inhibition”, “inhibit”, “inhibiting” and the like mean negatively affecting (e.g. decreasing) activity, expression or function relative to the activity, expression or function in the absence of an inhibitor. In certain aspects, inhibition can mean negatively affecting (e.g. decreasing) the concentration or levels of a biomolecule, such as a protein or mRNA, relative to the concentration or level of the biomolecule in the absence of an inhibitor. In certain aspects, inhibition includes, partially or totally, blocking stimulation, decreasing, preventing, or delaying activation; inactivating, desensitizing, or down-regulating signal transduction or enzymatic activity; or decreasing the amount of a biomolecule target (e.g., protein target or mRNA target). In certain aspects, inhibition refers to a reduction in the expression of a particular biomolecule target, such as a protein target (e.g., HMGCR protein) or an mRNA target (e.g., HMGCR mRNA). In certain aspects, inhibition refers to a reduction of amount of a target biomolecule (e.g., HMGCR protein or mRNA) resulting from a down-regulating protein expression (e.g. directly inhibiting translation or transcription). In certain aspects, inhibition refers to a reduction of activity of a target biomolecule (e.g., HMGCR protein or mRNA) from an indirect interaction (e.g., inhibiting or regulating other transcriptional or translational factors).

The term “inhibitor” also refers to a compound, composition, or substance capable of detectably negatively affecting (e.g. decreasing) activity, expression or function of a given protein or gene. For example, an inhibitor may decrease activity, expression or function by about 10%, 20%, 30%, 40%, 50%, 60%, 70%, 80%, 90% or greater in comparison to a control in the absence of the inhibitor. Inhibitors include, for example, synthetic or biological molecules, such as oligonucleotides. In some embodiments, the inhibitors include RNAi agent, e.g., siRNA agent, dsRNAi agent, or dsRNA agent.

As used herein, the “level or degree of inhibiting or decreasing expression” of a given gene refers to the at least partial suppression of the expression of a target gene (e.g., HMGCR), as manifested by a reduction of the amount of the target gene mRNA (e.g., HMGCR mRNA) or protein (e.g., HMGCR) encoded by the target gene, which may be isolated from or detected in a group of cells (“a first cell”) in which a target gene is transcribed and which has or have been treated such that the expression of a target gene is inhibited, as compared to group of cells substantially identical to the first cell but without treated (“control cells” or “a second cell”).

In some embodiments, the level or expression of the target gene (e.g., HMGCR) can be measured by evaluation of mRNA (e.g., via Northern blots or PCR). The effect of an RNAi agent on the target gene (e.g., HMGCR) expression can be determined by measuring the gene transcription rates (e.g., via Northern blots; or reverse transcriptase polymerase chain reaction or real-time polymerase chain reaction). In some embodiments, the degree of inhibition can be calculated as the following equation:

$$\frac{( mRNA in control cells ) - ( mRNA in treatedcells )}{( mRNA in control cells )} \cdot 100 \%$$

Alternatively, the degree of inhibition may be given in terms of a reduction of a parameter that is functionally linked to target gene (e.g., HMGCR) expression, e.g., the amount of protein encoded by a target gene (e.g., HMGCR), alteration in expression of the protein whose expression is dependent on the target gene (e.g., HMGCR), alteration in an activity of the enzyme (e.g., HMGCR) encoded by the target gene (e.g., HMGCR). In some embodiments, the level or expression of the protein (e.g., HMGCR) from the target gene can be evaluated by measuring the expressed protein amount (e.g., Western blots). In some embodiments, the level or expression of the protein from the target gene can be measured by the enzymatic assay (e.g., kinetic assay) of the protein.

As used herein, the term “down-regulate” or “down-regulating” refers to any statistically significant decrease in a biological activity and/or expression of the target protein (e.g., HMGCR), including full blocking of the activity (i.e., complete inhibition) and/or expression. For example, “down-regulation” can refer to a decrease of at least about 10%, 20%, 30%, 40%, 50%, 60%, 70%, 80%, 90% or 100% in target protein (e.g., HMGCR) level, activity and/or expression.

As used herein, the terms “salt” or “salts” refers to an acid addition or base addition salt of a compound of the present invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto. When both a basic group and an acid group are present in the same molecule, the compounds of the present invention may also form internal salts, e.g., zwitterionic molecules. In certain aspects, pharmaceutically acceptable acid addition salts can be formed with inorganic acids and organic acids. Examples of the inorganic acids from which salts can be derived include, for example, hydrochloric acid, hydrobromic acid, sulfuric acid, nitric acid, phosphoric acid, and the like. Examples of the organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. In certain aspects, the pharmaceutically acceptable base addition salts can be formed with inorganic and organic bases. Examples of the inorganic bases from which salts can be derived include, for example, ammonium salts and metals from columns I to XII of the periodic table, such as sodium, potassium, ammonium, calcium, magnesium, iron, silver, zinc, and copper; particularly suitable salts include ammonium, potassium, sodium, calcium and magnesium salts. Examples of the organic bases from which salts can be derived include, for example, primary, secondary, and tertiary amines, substituted amines including naturally occurring substituted amines, cyclic amines, basic ion exchange resins, and the like, such as organic amines include isopropylamine, benzathine, cholinate, diethanolamine, diethylamine, lysine, meglumine, piperazine and tromethamine.

In certain aspects, the term “pharmaceutically acceptable salt” as used herein may include the salts forms in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

As used herein, the term “treat,” “treating,” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient. In some embodiments, treating does not include preventing.

As used herein, the term “prevent”, “preventing” or “prevention” of any disease or disorder refers to the prophylactic treatment of the disease or disorder; or delaying the onset or progression of the disease or disorder.

The term “therapy,” as used herein refers to an application of one or more specific procedures used for the amelioration of at least one indicator or a disease or condition. In certain aspects, the specific procedure is the administration of one or more pharmaceutical or therapeutic agents.

The term “associated” or “associated with” in the context of a substance or substance activity or function associated with a disease (e.g. a protein associated disease, or HMGCR associated disease) means that the disease is caused by (in whole or in part), or a symptom of the disease is caused by (in whole or in part) the substance or substance activity or function (e.g., HMGCR activity or function). Thus, as used herein, what is described as “being associated” with a disease, if a causative agent, could be a target for treatment of the disease.

The term “HMGCR-associated disorder or disease,” as used herein refers to a disorder or disease that is caused by, or associated with, HMGCR gene expression or HMGCR protein production. For example, HMGCR-associated disorder or disease (e.g. hyperlipidemia, hypercholesterolemia or ASCVD) may be treated with a HMGCR modulator (e.g., gene silencing agent or down regulator) or HMGCR inhibitor, in the instance where HMGCR activity or function (e.g., enzyme activity in cholesterol synthesis) controls key step in the cholesterol synthesis or metabolism.

As used herein, the term “hyperlipidemia” refers to any disorder, disease or condition characterized by abnormal elevation of levels of any or all lipids, such as cholesterol and triglycerides, and/or lipoproteins in the blood or a condition that can lead to abnormal elevation of levels of any or all lipids and/or lipoproteins in the blood. In one embodiment, the hyperlipidemia is hypertriglyceridemia. As used herein, the term “hypertriglyceridemia” refers to a condition in which triglyceride levels are elevated, often caused or exacerbated by uncontrolled hyperlipidemia mellitus, obesity, and sedentary habits, e.g., when triglycerides in blood are greater than 1000-2000 mg/dL. As used herein the term “hypercholesterolemia” refers to a form of hyperlipidemia (elevated levels of lipids in the blood) in which there are high levels of cholesterol in the serum of a subject, e.g., at least about 240 mg/dL of total cholesterol.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “combination,” embraces mixtures of first and second dsRNAi agents. The term “combination,” also embraces first and second dsRNAi agents, which are formulated separately. In some embodiments, the first dsRNAi agent and/or the second dsRNAi agent are administered together with one or more additional therapeutic agents. In certain embodiments, the first dsRNAi agent is administered at the same time, prior to, or after the administration of the second dsRNAi agent. In certain embodiments, the first dsRNAi agent and the second dsRNAi agent are administered together. In some embodiments, the first dsRNAi agent is formulated with one or more additional therapeutic agents, optionally in the same pharmaceutical composition as the first dsRNAi agent. In some embodiments, the second dsRNAi agent is formulated with one or more additional therapeutic agents, optionally in the same pharmaceutical composition as the second dsRNAi agent. In some embodiments, the first dsRNAi agent and the second dsRNAi agent are formulated with one or more additional therapeutic agents, optionally in the same pharmaceutical composition as the first dsRNAi agent and the second dsRNAi agent. In some embodiments, the first dsRNAi agent (e.g., an HMGCR dsRNAi agent described herein) and the second dsRNAi agent (e.g., inclisiran) are formulated as a fixed dose combination (“FDC”).

Co-administration includes administering one active agent (e.g., RNAi agent) within 0.5, 1, 2, 4, 6, 8, 10, 12, 16, 20, or 24 hours of a second active agent (e.g. anticancer or antitumor agents). Also contemplated herein, are embodiments, where co-administration includes administering one active agent (e.g., dsRNAi agent or a therapeutic agent) within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In some embodiments, the active agents can be formulated separately.

In some embodiments, the first dsRNAi agent and the second dsRNAi agent are co-administered. Co-administration includes administering the first dsRNAi agent within 0.5, 1, 2, 4, 6, 8, 10, 12, 16, 20, or 24 hours of the second dsRNAi agent. Co-administration includes administering the first dsRNAi agent and the second dsRNAi agent simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including the first dsRNAi agent and the second dsRNAi agent. In some embodiments, the first dsRNAi agent and the second dsRNAi agent are formulated separately.

The terms “subject” and “patient” as used herein are used interchangeably. The term subject includes a human or non-human animal, preferably a vertebrate, and more preferably a mammal. In certain aspects, the subject is a human. In certain aspects, the subject is a human patient.

As used herein, a subject is “in need of” a treatment if such subject would benefit biologically, medically or in quality of life from such treatment.

The term “a therapeutically effective amount” of a compound (e.g., siRNA) as disclosed herein refers to an amount of the compound that will elicit the biological or medical response of a subject, for example, reduction or inhibition of an enzyme or a protein activity, or ameliorate symptoms, alleviate conditions, slow or delay disease progression, or prevent a disease, etc. In certain aspects, the term “a therapeutically effective amount” refers to the amount of the compound (e.g., siRNA) of the disclosure that, when administered to a subject, is effective to (1) at least partially alleviate, prevent and/or ameliorate a condition, or a disorder or a disease (i) mediated by the target gene (e.g., HMGCR), or (ii) associated with its activity, or (iii) characterized by activity (normal or abnormal) of the protein encoded by the target gene (e.g., HMGCR); or (2) reduce or inhibit the activity of the protein encoded by the target gene (e.g., HMGCR); or (3) reduce or inhibit the expression of the target gene (e.g., HMGCR). In certain aspects, the term “a therapeutically effective amount” refers to the amount of the compound that, when administered to a cell, or a tissue, or a non-cellular biological material, or a medium, is effective to at least partially reducing or inhibiting the activity of the protein encoded by the target gene (e.g., HMGCR); or at least partially reducing or inhibiting the expression of the protein (e.g., HMGCR) encoded by the target gene. The meaning of the term “a therapeutically effective amount” as illustrated in the above embodiment for the target gene expression also applies by the same means to any other relevant proteins/peptides/enzymes (e.g., HMGCR or other proteins relevant to cholesterol biosynthesis).

For any compound described herein, the therapeutically effective amount can be initially determined from cell culture assays. Target concentrations will be those concentrations of active compound(s) that are capable of achieving the methods described herein, as measured using the methods described herein or known in the art. Therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods established well within the capabilities of the ordinarily skilled artisan (e.g., physician or medical expert). An example of an “therapeutically effective amount” is an amount sufficient to contribute to the treatment, prevention, or reduction of a symptom or symptoms of a disease. For example, for the given parameter (e.g., biomarker), a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The term “control” or “control experiment” is used in accordance with its plain ordinary meaning and refers to an experiment in which the subjects or reagents of the experiment are treated as in a parallel experiment except for omission of a procedure, reagent, or variable of the experiment. Typically, a control is used as a standard of comparison in evaluating experimental effects. In some embodiments, a control is the measurement of the expression of a protein or mRNA (e.g., HMGCR) in the absence of RNAi agents as described herein.

The term “muscle side effect,” “muscle adverse effect,” or “muscle-related side effect” as used herein refers to a symptom or negative side effect, such as pain (e.g., ranging from mild discomfort to serious pain), weakness, soreness, tiredness, damage (e.g., rhabdomyolysis), or spasms, caused in or near muscles or muscle tissues. In some embodiments, the muscle side effect may be a drug-induced myopathies, for example, caused by taking a medicine (e.g., statin).

Unless defined otherwise, the chemical structures and formulae set forth herein are constructed according to the standard rules of chemical valency known in the chemical arts.

The term “alkyl,” by itself or as part of another substituent, means, unless otherwise stated, a straight (i.e., unbranched) or branched carbon chain (or carbon), or combination thereof, which is fully saturated (i.e., molecule by only single bonds) and include mono-, di- and multivalent radicals. As used herein, the alkyl is an uncyclized chain. The alkyl may include a designated number of carbons (e.g., C₁-C₁₀ means one to ten carbons). Examples of alkyl include, but are not limited to, groups such as C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl. For example, C₁₋₆ alkyl include, but are not limited to, methyl, ethyl, n-propyl, 1-methylethyl (iso-propyl), n-butyl, n-pentyl and 1,1-dimethylethyl (t-butyl), and their isomers.

A term “alkylene,” by itself or as part of another substituent, means, unless otherwise stated, a divalent radical derived from an alkyl, as exemplified, but not limited by, -CH₂CH₂CH₂CH₂—.

As used herein, the term “alkenyl,” by itself or as part of another substituent, means, unless otherwise stated, a straight (i.e., unbranched) or branched carbon chain (or carbon), or combination thereof, which is mono- or polyunsaturated (i.e., molecule including at least one double bond) and include mono-, di- and multivalent radicals. As used herein, the alkenyl is an uncyclized chain. Like the alkyl, the alkenyl may include a designated number of carbons (e.g., C₁-C₁₀ means one to ten carbons). Examples of alkenyl include, but are not limited to, groups such as C₁₋₃₀ alkenyl, C₁₋₂₅ alkenyl, C₁₋₂₀ alkenyl, C₁₋₁₅ alkenyl, C₁₋₁₂ alkenyl, C₁₋₁₀ alkenyl, C₁₋₈ alkenyl, C₁₋₆ alkenyl, C₁₋₄ alkenyl, or C₁₋₃ alkenyl. For example, C₂₋₆ alkenyl include, but are not limited to, ethenyl (vinyl), prop-1-enyl, but-1-enyl, pent-1-enyl, pent-4-enyl and penta-1,4-dienyl, and their isomers. A term “alkenylene,” by itself or as part of another substituent, means, unless otherwise stated, a divalent radical derived from an alkenyl, as exemplified, but not limited by, —CH═CHCH₂CH₂—.

As used herein, the term “alkynyl,” by itself or as part of another substituent, means, unless otherwise stated, a straight (i.e., unbranched) or branched carbon chain (or carbon), or combination thereof, which is mono- or polyunsaturated (i.e., molecule including at least one triple bond) and include mono-, di- and multivalent radicals. As used herein, the alkynyl is an uncyclized chain. Like the alkyl, the alkynyl may include a designated number of carbons (e.g., C₁-C₁₀ means one to ten carbons). Examples of alkynyl include, but are not limited to, groups such as C₁₋₃₀ alkynyl, C₁₋₂₅ alkynyl, C₁₋₂₀ alkynyl, C₁₋₁₅ alkynyl, C₁₋₁₂ alkynyl, C₁₋₁₀ alkynyl, C₁₋₈ alkynyl, C₁₋₆ alkynyl, C₁₋₄ alkynyl, or C₁₋₃ alkynyl. For example, C₂₋₆ alkynyl include, but are not limited to, alkynyl, and their isomers. A term “alkynyl,” by itself or as part of another substituent, means, unless otherwise stated, a divalent radical derived from an alkenyl, as exemplified, but not limited by, —CCH₂CH₂—.

As used herein, the term “alkoxy” refers to a radical of the formula —OR^{a} where R^{a} is an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as generally defined above. For example, C₁₋₆ alkoxy include, but are not limited to, methoxy, ethoxy, propoxy, isopropoxy, butoxy, isobutoxy, pentoxy, and hexoxy.

As used herein, the term “alkoxyalkyl” refers to a radical of the formula —R^{a}—O—R^{b} where each R^{a} and R^{b} is independently an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above and oxygen atom may be bonded to any carbon atom in either alkyl radical. For example, C₁₋₆alkoxy C₁₋₆alkyl include, but are not limited to, methoxy-methyl, methoxy-ethyl, ethoxy-ethyl, 1-ethoxy-propyl and 2-methoxy-butyl.

As used herein, the term “alkylcarbonyl” refers to a radical of the formula —C(═O)—R^{a} where R^{a} is an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above.

As used herein, the term “alkyl-carbonyl alkyl” refers to a radical of the formula —R^{a}—C(═O)—R^{b} where each R^{a} and R^{b} is independently an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₄ alkyl, or C₁₋₃ alkyl) radical as defined above. The carbon atom of the carbonyl group may be bonded to any carbon atom in either alkyl radical.

As used herein, the term “alkylaminocarbonyl” refers to a radical of the formula —C(═O)—NH—R^{a} where R^{a} is an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) as defined above.

As used herein, the term “alkoxycarbonyl” refers to a radical of the formula —C(═O)-O—R^{a} where R^{a} is an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above.

As used herein, the term “alkoxycarbonyl alkyl” refers to a radical of the formula -R^{a}—C(═O)—O—R^{b} where each R^{a} and R^{b} is independently an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above.

As used herein, the term “haloalkyl” refers to an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical, as defined above, substituted by one or more halo radicals, as defined above. Examples of halogen C₁₋₆alkyl include, but are not limited to, trifluoromethyl, difluoromethyl, fluoromethyl, trichloromethyl, 2,2,2-trifluoroethyl, 1,3-dibromopropan-2-yl, 3-bromo-2-fluoropropyl and 1,4,4-trifluorobutan-2-yl.

As used herein, the term “hydroxyalkyl” refers to an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above, wherein one of the hydrogen atoms of the alkyl radical is replaced by OH. Examples of hydroxyC₁₋₆ alkyl include, but are not limited to, hydroxy-methyl, 2-hydroxy-ethyl, 2-hydroxy-propyl, 3-hydroxy-propyl and 5-hydroxy-pentyl.

As used herein, the term “aminoalkyl” refers to an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above, wherein one of the hydrogen atoms of the C₁₋₆alkyl group is replaced by a primary amino group. Examples of amino C₁₋₆ alkyl include, but are not limited to, amino-methyl, 2-amino-ethyl, 2-amino-propyl, 3-amino-propyl, 3-amino-pentyl and 5-amino-pentyl.

As used herein, the term “alkylamino” refers to a radical of the formula —NH—R^{a} where R^{a} is an alkyl (e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl) radical as defined above.

The term “heteroalkyl,” by itself or in combination with another term, means, unless otherwise stated, a stable straight or branched chain, or combination thereof, which is fully saturated (i.e., molecule by only single bonds) and include mono-, di- and multivalent radicals, including at least one carbon atom and at least one heteroatom (e.g., O, N, S, Si, or P), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. The heteroalkyl may include a designated number of carbons and heteroatoms (e.g., “2 to 10 membered heteroalkyl” means two to 10 atoms including carbons and heteroatoms).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like).

As used herein, the term “heteroalkenyl,” by itself or as part of another substituent, means, unless otherwise stated, a straight (i.e., unbranched) or branched carbon chain (or carbon), or combination thereof, which is mono- or polyunsaturated (i.e., molecule including at least one double bond between carbon and carbon) and include mono-, di- and multivalent radicals. As used herein, the alkenyl is an uncyclized chain. Like the alkenyl, the heteroalkenyl may include a designated number of carbons and heteroatoms (e.g., “2 to 10 membered heteroalkenyl” means two to 10 atoms including carbons and heteroatoms).

As used herein, the term “heteroalkynyl,” by itself or as part of another substituent, means, unless otherwise stated, a straight (i.e., unbranched) or branched carbon chain (or carbon), or combination thereof, which is mono- or polyunsaturated (i.e., molecule including at least one triple bond between carbon and carbon) and include mono-, di- and multivalent radicals. As used herein, the alkynyl is an uncyclized chain. The heteroalkynyl may include a designated number of carbons and heteroatoms (e.g., “2 to 10 membered heteroalkynyl” means two to 10 atoms including carbons and heteroatoms).

For alkylene and heteroalkylene linking groups, no orientation of the linking group is implied by the direction in which the formula of the linking group is written. For example, the formula —C(O)₂R′— represents both —C(O)₂R′— and —R′C(O)₂—.

A “cycloalkylene” and a “heterocycloalkylene,” alone or as part of another substituent, means a divalent radical derived from a cycloalkyl and heterocycloalkyl, respectively. The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzooxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The symbol “ [symbol] ” denotes the point of attachment of a chemical moiety to the remainder of a molecule or chemical formula.

The term “oxo,” as used herein, means an oxygen that is double-bonded to a carbon atom.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, NR′C(O)R″, NR′C(O)—OR″, NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Certain compounds provided herein possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)-or (S)- or, as (D)- or (L)- for amino acids, and individual isomers are encompassed within the scope of the present disclosure. The compounds of provided herein do not include those that are known in art to be too unstable to synthesize and/or isolate. Compounds provided herein include those in racemic and optically pure forms. Optically active (R)- and (S)-, or (D)- and (L)-isomers may be prepared using chiral synthons or chiral reagents, or resolved using conventional techniques. When the compounds described herein contain olefinic bonds (vinyl group) and unless specified otherwise, it is intended that the compounds include both (E) and (Z) geometric isomers.

As used herein, the term “isomers” refers to compounds having the same number and kind of atoms, and hence the same molecular weight, but differing in respect to the structural arrangement or configuration of the atoms.

### RNAi Agents

In an aspect, the disclosure provides an RNAi agent including a double stranded RNA (dsRNA). In an aspect, also provided is a dsRNA interference (dsRNAi) agent that includes a dsRNA consisting of (i) a sense strand and (ii) an antisense strand, and a ligand attached to at least one of the sense strand and the antisense strand.

A dsRNA is a complex of ribonucleic acid (RNA) molecules formed in a duplex structure. In certain aspects, the dsRNA may be a short interfering RNA (siRNA) that has 10 to 30, or particularly 15-25 nucleotides in each RNA molecule, respectively, “passenger strand” and “guide strand”, and can be incorporated into an RNA-induced silencing complex (RISC). The siRNA is dissociated or unwounded in the RISC, and the passenger strand is degraded while the guide strand remains in the RISC pathway. The guide strand can subsequently bind to a mRNA molecule that includes a complementary sequence to the guide strand and induce or initiate cleavage or degradation of the mRNA molecule. In certain aspects, the mRNA encodes a target gene (e.g., mRNA transcript of a target gene) such that expression of the target gene is suppressed or inhibited through a post-transcriptional gene-silencing (“RNA silencing”). A guide RNA molecule has a complementary sequence to a target mRNA sequence and has anti-parallel orientation to the target gene, so it is interchangeably referred to as an antisense strand. A passenger RNA molecule forming a duplex with the guide RNA and having a complementary sequence to the guide strand (antisense strand) has the same orientation with the target mRNA sequence, so it is interchangeably referred to as an antisense strand.

HMGCR siRNA (Double Stranded RNA)

In an aspect, the disclosure provides a dsRNA interference (dsRNAi) agent that is capable of interacting or recruiting a target mRNA sequence, e.g., HMGCR target mRNA sequence, in the RISC thereby cleaving the target mRNA. The dsRNAi agent can silence HMGCR gene, e.g., by inhibiting, downregulating, or suppressing the expression of HMGCR gene. Gene-silencing (e.g., inhibiting, downregulating, or suppressing of the gene) may be assessed by a decrease in an absolute or relative level of one or more variables that are associated with HMGCR expression compared with a control level. The control level may be any type obtained from, e.g., a pre-dose baseline level, or a level determined from a similar subject, cell, or untreated or treated subject with inactive agents (e.g., PBS buffer). In some embodiments, the level of silencing the HMGCR may be demonstrated by a reduction of the amount of a total HMGCR mRNA in a cell. In some embodiments, the level of silencing the HMGCR may be demonstrated by a reduction of the amount of a total HMGCR protein in a cell.

In an aspect, the dsRNAi agent as described herein can inhibit expression of the HMGCR gene (e.g., human HMGCR) by at least about 10%, about 15%, about 20%, about 25%, about 30%, about 40%, about 50%, about 55%, about 60%, about 65%, about 70%, about 75%, about 80%, about 85%, about 90%, or about 95% based on the expression level of the HMGCR gene in untreated cell or subject. In some embodiments, expression of the HMGCR gene (e.g., human HMGCR) is inhibited by at least about 10%, about 15%, about 20%, about 25%, about 30%, about 40%, about 50%, about 55%, about 60%, about 65%, about 70%, about 75%, about 80%, about 85%, about 90%, or about 95% based on the expression level of the HMGCR gene in untreated cell or subject. In some embodiments, expression of the HMGCR gene (e.g., human HMGCR) is inhibited by at least about 10% based on the expression level of the HMGCR gene in untreated cell or subject. In some embodiments, expression of the HMGCR gene (e.g., human HMGCR) is inhibited by at least about 20% based on the expression level of the HMGCR gene in untreated cell or subject. In some embodiments, expression of the HMGCR gene (e.g., human HMGCR) is inhibited by at least about 30% based on the expression level of the HMGCR gene in untreated cell or subject. In some embodiments, expression of the HMGCR gene (e.g., human HMGCR) is inhibited by at least about 40% based on the expression level of the HMGCR gene in untreated cell or subject. In some embodiments, expression of the HMGCR gene (e.g., human HMGCR) is inhibited by at least about 50% based on the expression level of the HMGCR gene in untreated cell or subject. In some embodiments, expression of the HMGCR gene (e.g., human HMGCR) is inhibited by at least about 60% based on the expression level of the HMGCR gene in untreated cell or subject. In some embodiments, expression of the HMGCR gene (e.g., human HMGCR) is inhibited by at least about 70% based on the expression level of the HMGCR gene in untreated cell or subject.

In an aspect, the dsRNAi agent as described herein can decrease expression of the HMGCR gene (e.g., human HMGCR) by at least about 10%, about 15%, about 20%, about 25%, about 30%, about 40%, about 50%, about 55%, about 60%, about 65%, about 70%, about 75%, about 80%, about 85%, about 90%, or about 95% based on the expression level of the HMGCR gene in untreated cell or subject. In some embodiments, expression of the HMGCR gene (e.g., human HMGCR) is decreased by at least about 10%, about 15%, about 20%, about 25%, about 30%, about 40%, about 50%, about 55%, about 60%, about 65%, about 70%, about 75%, about 80%, about 85%, about 90%, or about 95% based on the expression level of the HMGCR gene in untreated cell or subject. In some embodiments, expression of the HMGCR gene (e.g., human HMGCR) is decreased by at least about 10% based on the expression level of the HMGCR gene in untreated cell or subject. In some embodiments, expression of the HMGCR gene (e.g., human HMGCR) is decreased by at least about 20% based on the expression level of the HMGCR gene in untreated cell or subject. In some embodiments, expression of the HMGCR gene (e.g., human HMGCR) is decreased by at least about 30% based on the expression level of the HMGCR gene in untreated cell or subject. In some embodiments, expression of the HMGCR gene (e.g., human HMGCR) is decreased by at least about 40% based on the expression level of the HMGCR gene in untreated cell or subject. In some embodiments, expression of the HMGCR gene (e.g., human HMGCR) is decreased by at least about 50% based on the expression level of the HMGCR gene in untreated cell or subject. In some embodiments, expression of the HMGCR gene (e.g., human HMGCR) is decreased by at least about 60% based on the expression level of the HMGCR gene in untreated cell or subject. In some embodiments, expression of the HMGCR gene (e.g., human HMGCR) is decreased by at least about 70% based on the expression level of the HMGCR gene in untreated cell or subject.

In an aspect, the dsRNAi agent as described herein can suppress expression of the HMGCR gene (e.g., human HMGCR) by at least about 10%, about 15%, about 20%, about 25%, about 30%, about 40%, about 50%, about 55%, about 60%, about 65%, about 70%, about 75%, about 80%, about 85%, about 90%, or about 95% based on the expression level of the HMGCR gene in untreated cell or subject. In some embodiments, expression of the HMGCR gene (e.g., human HMGCR) is suppressed by at least about 10%, about 15%, about 20%, about 25%, about 30%, about 40%, about 50%, about 55%, about 60%, about 65%, about 70%, about 75%, about 80%, about 85%, about 90%, or about 95% based on the expression level of the HMGCR gene in untreated cell or subject. In some embodiments, expression of the HMGCR gene (e.g., human HMGCR) is suppressed by at least about 10% based on the expression level of the HMGCR gene in untreated cell or subject. In some embodiments, expression of the HMGCR gene (e.g., human HMGCR) is suppressed by at least about 20% based on the expression level of the HMGCR gene in untreated cell or subject. In some embodiments, expression of the HMGCR gene (e.g., human HMGCR) is suppressed by at least about 30% based on the expression level of the HMGCR gene in untreated cell or subject. In some embodiments, expression of the HMGCR gene (e.g., human HMGCR) is suppressed by at least about 40% based on the expression level of the HMGCR gene in untreated cell or subject. In some embodiments, expression of the HMGCR gene (e.g., human HMGCR) is suppressed by at least about 50% based on the expression level of the HMGCR gene in untreated cell or subject. In some embodiments, expression of the HMGCR gene (e.g., human HMGCR) is suppressed by at least about 60% based on the expression level of the HMGCR gene in untreated cell or subject. In some embodiments, expression of the HMGCR gene (e.g., human HMGCR) is suppressed by at least about 70% based on the expression level of the HMGCR gene in untreated cell or subject.

In some embodiments, inhibition of the expression of the HMGCR gene may be manifested by a reduction of the amount of mRNA expressed in a first cell or a first group of cells obtained from a subject that has been treated, e.g., by contacting the cell or by administering the dsRNAi agent as described herein, as compared to a second cell or a second group of cells obtained from a subject that has not been treated but is identical to the first cell or the first group of cells. For example, the level of gene-silencing (e.g., inhibiting, downregulating, or suppressing of the gene) of the HMGCR (e.g., human HMGCR) may be presented as a percentage of remaining mRNA in the treated cells (first cell or group of cells) compared to the mRNA amount in the control (untreated) cells, as shown in the following equation:

$$\frac{( mRNA in control cells ) - ( mRNA in treated cells )}{( mRNA in control cells )} \cdot 100 \% .$$

In some embodiments, the level of gene-silencing (e.g., inhibiting, downregulating, or suppressing of the gene) of the HMGCR (e.g., human HMGCR) may be assessed by measuring a parameter or biomarker, e.g., human HMGCR protein level, in a biological sample (e.g., e.g., a blood, serum or liver tissue obtained from a subject), which may be treated or untreated. Conventional analytical methods as known in the art such as electrophoresis (e.g., SDS or capillary electrophoresis), chromatography (e.g., high performance liquid chromatography (HPLC)), spectroscopy, western blotting, enzyme-linked immunosorbent assays (ELISAs), immunofluorescent assays, electrochemiluminescence assays, and the like can be used without limitation, but examples are not limited thereto. In some embodiments, reduced level of gene-silencing (e.g., inhibiting, downregulating, or suppressing of the gene) of the HMGCR (e.g., human HMGCR) may be observed or assessed by in a liver (tissue) biopsy of the treated subject.

In certain aspects, the dsRNAi agent is a free acid. In certain aspects, the dsRNAi agent is in a salt form (e.g., a pharmaceutically acceptable salt form. It will be understood that references to dsRNAi agent are meant to also include the pharmaceutically acceptable salts of the dsRNAi agent. If the dsRNAi agent has, for example, at least one basic center, they can form acid addition salts. Corresponding acid addition salts can also be formed having, if desired, an additionally present basic center. Active substances having an acid group, e.g., COOH, can form salts with bases. The dsRNAi agent or pharmaceutically acceptable salts thereof may also be used in form of a hydrate or include other solvents used for crystallization. In some embodiments, the RNAi agent is a sodium salt. In some embodiments, the dsRNAi agent is in a salt form (e.g., a pharmaceutically acceptable salt form), where the salt is sodium (Na⁺), ammonium (NH₄⁺), calcium (Ca²⁺), iron (Fe²⁺ or Fe³⁺), magnesium (Mg²⁺), potassium (K⁺), pyridinium (C₅H₅NH⁺), quaternary ammonium (NR₄⁺, R being an alkyl group or an aryl group as described herein), or copper (Cu²⁺).

In an aspect, the disclosure provides a dsRNA having sequences (e.g., antisense strand sequence) that can recognize a specific region of a HMGCR mRNA (e.g., human HMGCR mRNA) and lead cleavage of the HMGCR mRNA and silencing of the gene. The dsRNA includes a sense strand and an antisense strand and each strand may range from 12 to 30 nucleotides in length. In some embodiments, each strand may have 15 to 30 nucleotides in length. In some embodiments, each strand may have 15 to 25 nucleotides in length. In some embodiments, the antisense strand may have 15 to 25 nucleotides in length. In some embodiments, the sense strand may have 15 to 25 nucleotides in length. In some embodiments, the antisense strand may have 15 to 23 nucleotides in length. In some embodiments, the sense strand may have 15 to 23 nucleotides in length. In some embodiments, the antisense strand may have 18 to 25 nucleotides in length. In some embodiments, the sense strand may have 18 to 25 nucleotides in length.

In some embodiments, the sense strand may have 19 to 23 nucleotides in length. In some embodiments, the sense strand may have 21 to 23 nucleotides in length. In some embodiments, the sense strand may have 19 nucleotides in length. In some embodiments, the sense strand may have 20 nucleotides in length. In some embodiments, the sense strand may have 21 nucleotides in length. In some embodiments, the sense strand may have 22 nucleotides in length. In some embodiments, the sense strand may have 23 nucleotides in length.

In some embodiments, the antisense strand may have 19 to 25 nucleotides in length. In some embodiments, the antisense strand may have 19 to 23 nucleotides in length. In some embodiments, the antisense strand may have 21 to 23 nucleotides in length. In some embodiments, the antisense strand may have 23 to 25 nucleotides in length. In some embodiments, the antisense strand may have 19 nucleotides in length. In some embodiments, the antisense strand may have 20 nucleotides in length. In some embodiments, the antisense strand may have 21 nucleotides in length. In some embodiments, the antisense strand may have 22 nucleotides in length. In some embodiments, the antisense strand may have 23 nucleotides in length. In some embodiments, the antisense strand may have 24 nucleotides in length. In some embodiments, the antisense strand may have 25 nucleotides in length.

In some embodiments, the sense strand is 21 to 23 nucleotides in length and the antisense strand is 23 to 25 nucleotides in length. In some embodiments, the sense strand is 21 nucleotides in length and the antisense strand is 23 nucleotides in length. In some embodiments, the sense strand is 22 nucleotides in length and the antisense strand is 24 nucleotides in length. In some embodiments, the sense strand is 23 nucleotides in length and the antisense strand is 25 nucleotides in length.

In an aspect, a dsRNA as described herein forms a double-stranded (or “duplex”) region made between a sense strand and an antisense strand and having 10 to 25 nucleotide pairs in length. The double stranded or duplex region are loaded into the RISC complex and subsequent specific degradation of the sense strand occurs during the RISC pathway. In some embodiments, the double stranded region has 10 nucleotide base pairs in length. In some embodiments, the double stranded region has 11 nucleotide base pairs in length. In some embodiments, the double stranded region has 12 nucleotide base pairs in length. In some embodiments, the double stranded region has 13 nucleotide base pairs in length. In some embodiments, the double stranded region has 14 nucleotide base pairs in length. In some embodiments, the double stranded region has 15 nucleotide base pairs in length. In some embodiments, the double stranded region has 16 nucleotide base pairs in length. In some embodiments, the double stranded region has 17 nucleotide base pairs in length. In some embodiments, the double stranded region has 18 nucleotide base pairs in length. In some embodiments, the double stranded region has 19 nucleotide base pairs in length. In some embodiments, the double stranded region has 20 nucleotide base pairs in length. In some embodiments, the double stranded region has 21 nucleotide base pairs in length. In some embodiments, the double stranded region has 22 nucleotide base pairs in length. In some embodiments, the double stranded region has 23 nucleotide base pairs in length.

In an aspect, a dsRNA as described herein may include at least one single-stranded nucleotide overhang, for example, for increasing in vivo effectiveness of the dsRNA and having substantially improved inhibition of the target genes. In certain aspects, the dsRNA may contain one or more extra nucleotides constituting overhang regions that locate other than the double stranded region at the 3′-end, 5′-end, or both ends of either stand or both strands (sense and antisense strands). In some embodiments, the overhang region may exist at the 3′-end, 5′-end, or both ends of the sense strand. In some embodiments, the overhang region may exist at the 3′-end, 5′-end, or both ends of the antisense strand. In some embodiments, the antisense strand may have a greater length than a length in the sense strand. In some embodiments, the antisense strand may have a shorter length than a length in the sense strand.

In some embodiments, the dsRNA may contain one or more extra nucleotides constituting overhang regions at the 3′-end, 5′-end, or both ends of the antisense strand. In some embodiments, the overhang region in the antisense strand may consist of 1-6 nucleotides in length, for example, 1 nucleotide, 2 nucleotides, 3 nucleotides, 4 nucleotides, 5 nucleotides, or 6 nucleotides in length. In some embodiments, the dsRNA may contain one or more extra nucleotides constituting overhang regions at the 3′-end, 5′-end, or both ends of the sense strand. In some embodiments, the overhang region may consist of 1-6 nucleotides in length, for example, 1 nucleotide, 2 nucleotides, 3 nucleotides, 4 nucleotides, 5 nucleotides, or 6 nucleotides in length.

In some embodiments, the antisense strand may include one-nucleotide overhang at the 5′ end. In some embodiments, the antisense strand may include one-nucleotide overhang at the 3′ end. In some embodiments, the antisense strand may include two-nucleotides overhang. In some embodiments, the antisense contains two-nucleotides overhang at the 5′ end. In some embodiments, the antisense contains two-nucleotides overhang at the 3′ end. In some embodiments, the antisense contains one-nucleotide overhang at the 5′ end and one-nucleotide overhang at the 3′ end. In some embodiments, the antisense strand may include three-nucleotide overhang. In some embodiments, the antisense contains three-nucleotides overhang at the 5′ end. In some embodiments, the antisense contains three-nucleotides overhang at the 3′ end. In some embodiments, the antisense contains two-nucleotides overhang at the 5′ end and one-nucleotide overhang at the 3′ end. In some embodiments, the antisense contains two nucleotides overhang at the 3′ end and one-nucleotide overhang at the 5′ end.

In certain aspects, a dsRNA as described herein may include at least one blunt end, e.g., for increasing in vivo stability with resistance to degradation in physiological surroundings. In some embodiments, the dsRNA may have a blunt end at the 3′-end, 5′-end, or both ends of the duplex. In some embodiments, the dsRNA includes one overhang (e.g., at 3′ end of antisense strand) and one blunt end (e.g., at 5′ end of antisense strand). In some embodiments, the dsRNA includes a blunt end at the 5′-end of the sense strand (and at 3′ end of the antisense strand) and contain overhang nucleotide(s) at the other end. In some embodiments, the dsRNA may have a blunt end at the 3′-end of the sense strand (and at 5′ end of the antisense strand) and contain overhang nucleotide(s) at the other end.

The sequences of the single strands (i.e., sense strand and antisense strand) of the dsRNA can be selected by selecting a target region and a length in the HMGCR mRNA. In certain aspects, a dsRNA as described herein may target a nucleotide region selected from regions of (i) 50-250 and (ii) 2400-2600 of a human HMGCR mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 811 (human HMGCR isoform, transcript variant 1, mRNA (GenBank: NM_000859.3)). In some embodiments, the target region is selected from regions of (i) 100-200 and (ii) 2500-2600 of a human HMGCR mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 811 (human HMGCR isoform, transcript variant 1, mRNA (GenBank: NM_000859.3)).

In certain aspects, an antisense strand of the dsRNA as described herein targets a nucleotide region selected from regions of (i) 50-250 and (ii) 2400-2600 of a human HMGCR mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 811 (human HMGCR isoform, transcript variant 1, mRNA (GenBank: NM_000859.3)). In some embodiments, the antisense strand of the dsRNA as described herein targets a nucleotide region selected from regions of (i) 100-200 and (ii) 2500-2600 of a human HMGCR mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 811 (human HMGCR isoform, transcript variant 1, mRNA (GenBank: NM_000859.3)).

In some embodiments, the antisense strand targets a region of 50-250th nucleotides in a human HMGCR mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 811 (human HMGCR isoform, transcript variant 1, mRNA (GenBank: NM_000859.3)). In some embodiments, the antisense strand targets a region of 100-200th nucleotides in a human HMGCR mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 811 (human HMGCR isoform, transcript variant 1, mRNA (GenBank: NM_000859.3)). In some embodiments, the antisense strand targets a region of 100-150th nucleotides in a human HMGCR mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 811 (human HMGCR isoform, transcript variant 1, mRNA (GenBank: NM_000859.3)).

In some embodiments, the antisense strand targets a region of 2400-2600th nucleotides in a human HMGCR mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 811 (human HMGCR isoform, transcript variant 1, mRNA (GenBank: NM_000859.3)). In some embodiments, the antisense strand targets a region of 2500-2600th nucleotides in a human HMGCR mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 811 (human HMGCR isoform, transcript variant 1, mRNA (GenBank: NM_000859.3)). In some embodiments, the antisense strand targets a region of 2550-2600th nucleotides in a human HMGCR mRNA sequence that has at least about 85% (e.g., about 85%, about 86%, about 87%, about 88%, about 89%, about 90%, about 91%, about 92%, about 93%, about 94%, about 95%, about 96%, about 97%, about 98%, about 99% or 100%) identity to SEQ ID NO: 811 (human HMGCR isoform, transcript variant 1, mRNA (GenBank: NM_000859.3)).

In certain aspects, the target HMGCR mRNA sequence may range from 12 to 30 nucleotides, from 15 to 30 nucleotides, from 18 to 30 nucleotides, from 18 to 25 nucleotides, from 18 to 23 nucleotides. In some embodiments, the target HMGCR mRNA sequence may have 15 nucleotides in length. In some embodiments, the target HMGCR mRNA sequence may have 16 nucleotides in length. In some embodiments, the target HMGCR mRNA sequence may have 17 nucleotides in length. In some embodiments, the target HMGCR mRNA sequence may have 18 nucleotides in length. In some embodiments, the target HMGCR mRNA sequence may have 19 nucleotides in length. In some embodiments, the target HMGCR mRNA sequence may have 20 nucleotides in length. In some embodiments, the target HMGCR mRNA sequence may have 21 nucleotides in length. In some embodiments, the target HMGCR mRNA sequence may have 22 nucleotides in length. In some embodiments, the target HMGCR mRNA sequence may have 23 nucleotides in length.

In certain aspects, example dsRNA sequences including sense strands and antisense strands targeting the above indicated HMGCR mRNA (SEQ ID NO: 811, or GenBank: NM_000859.3) are in Table 1.

TABLE 1

| | | | SEQ | | SEQ |
|---|---|---|---|---|---|
| SIRNA | | | ID | | ID |
| No | position | Sense Strand | NO: | Antisense Strand | NO: |
| 1 | 122 | ACAAUGUUGUCAAGACUUUUU | 1 | AAAAAGUCUUGACAACAUUGUAG | 406 |
| 2 | 125 | AUGUUGUCAAGACUUUUUCGA | 2 | UCGAAAAAGUCUUGACAACAUUG | 407 |
| 3 | 126 | UGUUGUCAAGACUUUUUCGAA | 3 | UUCGAAAAAGUCUUGACAACAUU | 408 |
| 4 | 127 | GUUGUCAAGACUUUUUCGAAU | 4 | AUUCGAAAAAGUCUUGACAACAU | 409 |
| 5 | 130 | GUCAAGACUUUUUCGAAUGCA | 5 | UGCAUUCGAAAAAGUCUUGACAA | 410 |
| 6 | 131 | UCAAGACUUUUUCGAAUGCAU | 6 | AUGCAUUCGAAAAAGUCUUGACA | 411 |
| 7 | 133 | AAGACUUUUUCGAAUGCAUGA | 7 | UCAUGCAUUCGAAAAAGUCUUGA | 412 |
| 8 | 164 | GCCUCCCAUCCCUGGGAAGUA | 8 | UACUUCCCAGGGAUGGGAGGCCA | 413 |
| 9 | 167 | UCCCAUCCCUGGGAAGUCAUA | 9 | UAUGACUUCCCAGGGAUGGGAGG | 414 |
| 10 | 199 | GACACUGACCAUCUGCAUGAU | 10 | AUCAUGCAGAUGGUCAGUGUCAC | 415 |
| 11 | 222 | CCAUGAACAUGUUUACUGGUA | 11 | UACCAGUAAACAUGUUCAUGGAC | 416 |
| 12 | 228 | ACAUGUUUACUGGUAACAAUA | 12 | UAUUGUUACCAGUAAACAUGUUC | 417 |
| 13 | 229 | CAUGUUUACUGGUAACAAUAA | 13 | UUAUUGUUACCAGUAAACAUGUU | 418 |
| 14 | 230 | AUGUUUACUGGUAACAAUAAA | 14 | UUUAUUGUUACCAGUAAACAUGU | 419 |
| 15 | 244 | CAAUAAGAUCUGUGGUUGGAA | 15 | UUCCAACCACAGAUCUUAUUGUU | 420 |
| 16 | 246 | AUAAGAUCUGUGGUUGGAAUU | 16 | AAUUCCAACCACAGAUCUUAUUG | 421 |
| 17 | 247 | UAAGAUCUGUGGUUGGAAUUA | 17 | UAAUUCCAACCACAGAUCUUAUU | 422 |
| 18 | 275 | CCAAAGUUUGAAGAGGAUGUU | 18 | AACAUCCUCUUCAAACUUUGGAC | 423 |
| 19 | 277 | AAAGUUUGAAGAGGAUGUUUU | 19 | AAAACAUCCUCUUCAAACUUUGG | 424 |
| 20 | 295 | UUUGAGCAGUGACAUUAUAAU | 20 | AUUAUAAUGUCACUGCUCAAAAC | 425 |
| 21 | 302 | AGUGACAUUAUAAUUCUGACA | 21 | UGUCAGAAUUAUAAUGUCACUGC | 426 |
| 22 | 305 | GACAUUAUAAUUCUGACAAUA | 22 | UAUUGUCAGAAUUAUAAUGUCAC | 427 |
| 23 | 308 | AUUAUAAUUCUGACAAUAACA | 23 | UGUUAUUGUCAGAAUUAUAAUGU | 428 |
| 24 | 313 | AAUUCUGACAAUAACACGAUA | 24 | UAUCGUGUUAUUGUCAGAAUUAU | 429 |
| 25 | 313 | UUCUGACAAUAACACGAUGCA | 25 | UGCAUCGUGUUAUUGUCAGAAUU | 430 |
| 26 | 316 | UCUGACAAUAACACGAUGCAU | 26 | AUGCAUCGUGUUAUUGUCAGAAU | 431 |
| 27 | 317 | CUGACAAUAACACGAUGCAUA | 27 | UAUGCAUCGUGUUAUUGUCAGAA | 432 |
| 28 | 318 | UGACAAUAACACGAUGCAUAA | 28 | UUAUGCAUCGUGUUAUUGUCAGA | 433 |
| 29 | 340 | CAUCCUGUAUAUUUACUUCCA | 29 | UGGAAGUAAAUAUACAGGAUGGC | 434 |
| 30 | 346 | GUAUAUUUACUUCCAGUUCCA | 30 | UGGAACUGGAAGUAAAUAUACAG | 435 |
| 31 | 357 | UCCAGUUCCAGAAUUUACGUA | 31 | UACGUAAAUUCUGGAACUGGAAG | 436 |
| 32 | 360 | AGUUCCAGAAUUUACGUCAAA | 32 | UUUGACGUAAAUUCUGGAACUGG | 437 |
| 33 | 362 | UUCCAGAAUUUACGUCAACUU | 33 | AAGUUGACGUAAAUUCUGGAACU | 438 |
| 34 | 364 | CCAGAAUUUACGUCAACUUGA | 34 | UCAAGUUGACGUAAAUUCUGGAA | 439 |
| 35 | 365 | CAGAAUUUACGUCAACUUGGA | 35 | UCCAAGUUGACGUAAAUUCUGGA | 440 |
| 36 | 366 | AGAAUUUACGUCAACUUGGAU | 36 | AUCCAAGUUGACGUAAAUUCUGG | 441 |
| 37 | 370 | UUUACGUCAACUUGGAUCAAA | 37 | UUUGAUCCAAGUUGACGUAAAUU | 442 |
| 38 | 371 | UUACGUCAACUUGGAUCAAAA | 38 | UUUUGAUCCAAGUUGACGUAAAU | 443 |
| 39 | 434 | UUUGUAUUCAGUACAGUUGUA | 39 | UACAACUGUACUGAAUACAAAAC | 444 |
| 40 | 439 | AUUCAGUACAGUUGUCAUUCA | 40 | UGAAUGACAACUGUACUGAAUAC | 445 |
| 41 | 445 | UACAGUUGUCAUUCACUUCUU | 41 | AAGAAGUGAAUGACAACUGUACU | 446 |
| 42 | 450 | UUGUCAUUCACUUCUUAGACA | 42 | UGUCUAAGAAGUGAAUGACAACU | 447 |
| 43 | 451 | UGUCAUUCACUUCUUAGACAA | 43 | UUGUCUAAGAAGUGAAUGACAAC | 448 |
| 44 | 456 | UUCACUUCUUAGACAAAGAAU | 44 | AUUCUUUGUCUAAGAAGUGAAUG | 449 |
| 45 | 461 | UUCUUAGACAAAGAAUUGACA | 45 | UGUCAAUUCUUUGUCUAAGAAGU | 450 |
| 46 | 466 | AGACAAAGAAUUGACAGGCUU | 46 | AAGCCUGUCAAUUCUUUGUCUAA | 451 |
| 47 | 469 | CAAAGAAUUGACAGGCUUGAA | 47 | UUCAAGCCUGUCAAUUCUUUGUC | 452 |
| 48 | 543 | UAGCAAAGUUUGCCCUCAGUU | 48 | AACUGAGGGCAAACUUUGCUAAU | 453 |
| 49 | 561 | GUUCCAACUCACAGGAUGAAA | 49 | UUUCAUCCUGUGAGUUGGAACUG | 454 |
| 50 | 587 | GAAAAUAUUGCUCGUGGAAUA | 50 | UAUUCCACGAGCAAUAUUUUCCC | 455 |
| 51 | 588 | AAAAUAUUGCUCGUGGAAUGA | 51 | UCAUUCCACGAGCAAUAUUUUCC | 456 |
| 52 | 589 | AAAUAUUGCUCGUGGAAUGGA | 52 | UCCAUUCCACGAGCAAUAUUUUC | 457 |
| 53 | 590 | AAUAUUGCUCGUGGAAUGGCA | 53 | UGCCAUUCCACGAGCAAUAUUUU | 458 |
| 54 | 593 | AUUGCUCGUGGAAUGGCAAUU | 54 | AAUUGCCAUUCCACGAGCAAUAU | 459 |
| 55 | 595 | UGCUCGUGGAAUGGCAAUUUU | 55 | AAAAUUGCCAUUCCACGAGCAAU | 460 |
| 56 | 596 | GCUCGUGGAAUGGCAAUUUUA | 56 | UAAAAUUGCCAUUCCACGAGCAA | 461 |
| 57 | 600 | GUGGAAUGGCAAUUUUAGGUA | 57 | UACCUAAAAUUGCCAUUCCACGA | 462 |
| 58 | 620 | CCUACGUUUACCCUCGAUGCU | 58 | AGCAUCGAGGGUAAACGUAGGAC | 463 |
| 59 | 621 | CUACGUUUACCCUCGAUGCUA | 59 | UAGCAUCGAGGGUAAACGUAGGA | 464 |
| 60 | 639 | CUCUUGUUGAAUGUCUUGUGA | 60 | UCACAAGACAUUCAACAAGAGCA | 465 |
| 61 | 641 | CUUGUUGAAUGUCUUGUGAUU | 61 | AAUCACAAGACAUUCAACAAGAG | 466 |
| 62 | 644 | GUUGAAUGUCUUGUGAUUGGA | 62 | UCCAAUCACAAGACAUUCAACAA | 467 |
| 63 | 683 | GUACGUCAGCUUGAAAUUAUA | 63 | UAUAAUUUCAAGCUGACGUACCC | 468 |
| 64 | 684 | UACGUCAGCUUGAAAUUAUGU | 64 | ACAUAAUUUCAAGCUGACGUACC | 469 |
| 65 | 688 | UCAGCUUGAAAUUAUGUGCUA | 65 | UAGCACAUAAUUUCAAGCUGACG | 470 |
| 66 | 693 | UUGAAAUUAUGUGCUGCUUUA | 66 | UAAAGCAGCACAUAAUUUCAAGC | 471 |
| 67 | 697 | AAUUAUGUGCUGCUUUGGCUA | 67 | UAGCCAAAGCAGCACAUAAUUUC | 472 |
| 68 | 710 | UUUGGCUGCAUGUCAGUUCUU | 68 | AAGAACUGACAUGCAGCCAAAGC | 473 |
| 69 | 729 | UUGCCAACUACUUCGUGUUCA | 69 | UGAACACGAAGUAGUUGGCAAGA | 474 |
| 70 | 730 | UGCCAACUACUUCGUGUUCAU | 70 | AUGAACACGAAGUAGUUGGCAAG | 475 |
| 71 | 734 | AACUACUUCGUGUUCAUGACU | 71 | AGUCAUGAACACGAAGUAGUUGG | 476 |
| 72 | 736 | CUACUUCGUGUUCAUGACUUU | 72 | AAAGUCAUGAACACGAAGUAGUU | 477 |
| 73 | 738 | ACUUCGUGUUCAUGACUUUCU | 73 | AGAAAGUCAUGAACACGAAGUAG | 478 |
| 74 | 739 | CUUCGUGUUCAUGACUUUCUU | 74 | AAGAAAGUCAUGAACACGAAGUA | 479 |
| 75 | 743 | GUGUUCAUGACUUUCUUCCCA | 75 | UGGGAAGAAAGUCAUGAACACGA | 480 |
| 76 | 761 | CCAGCUUGUGUGUCCUUGGUA | 76 | UACCAAGGACACACAAGCUGGGA | 481 |
| 77 | 766 | UUGUGUGUCCUUGGUAUUAGA | 77 | UCUAAUACCAAGGACACACAAGC | 482 |
| 78 | 779 | GUAUUAGAGCUUUCUCGGGAA | 78 | UUCCCGAGAAAGCUCUAAUACCA | 483 |
| 79 | 800 | AGCCGCGAGGGUCGUCCAAUU | 79 | AAUUGGACGACCCUCGCGGCUUU | 484 |
| 80 | 801 | GCCGCGAGGGUCGUCCAAUUU | 80 | AAAUUGGACGACCCUCGCGGCUU | 485 |
| 81 | 836 | UUUGCCCGAGUUUUAGAAGAA | 81 | UUCUUCUAAAACUCGGGCAAAAU | 486 |
| 82 | 839 | GCCCGAGUUUUAGAAGAAGAA | 82 | UUCUUCUUCUAAAACUCGGGCAA | 487 |
| 83 | 876 | CUGUAACUCAGAGGGUCAAGA | 83 | UCUUGACCCUCUGAGUUACAGGA | 488 |
| 84 | 879 | UAACUCAGAGGGUCAAGAUGA | 84 | UCAUCUUGACCCUCUGAGUUACA | 489 |
| 85 | 880 | AACUCAGAGGGUCAAGAUGAU | 85 | AUCAUCUUGACCCUCUGAGUUAC | 490 |
| 86 | 882 | CUCAGAGGGUCAAGAUGAUUA | 86 | UAAUCAUCUUGACCCUCUGAGUU | 491 |
| 87 | 883 | UCAGAGGGUCAAGAUGAUUAU | 87 | AUAAUCAUCUUGACCCUCUGAGU | 492 |
| 88 | 890 | GUCAAGAUGAUUAUGUCUCUA | 88 | UAGAGACAUAAUCAUCUUGACCC | 493 |
| 89 | 902 | AUGUCUCUAGGCUUGGUUCUU | 89 | AAGAACCAAGCCUAGAGACAUAA | 494 |
| 90 | 904 | GUCUCUAGGCUUGGUUCUUGU | 90 | ACAAGAACCAAGCCUAGAGACAU | 495 |
| 91 | 909 | UAGGCUUGGUUCUUGUUCAUA | 91 | UAUGAACAAGAACCAAGCCUAGA | 496 |
| 92 | 919 | UCUUGUUCAUGCUCACAGUCA | 92 | UGACUGUGAGCAUGAACAAGAAC | 497 |
| 93 | 927 | AUGCUCACAGUCGCUGGAUAA | 93 | UUAUCCAGCGACUGUGAGCAUGA | 498 |
| 94 | 928 | UGCUCACAGUCGCUGGAUAGA | 94 | UCUAUCCAGCGACUGUGAGCAUG | 499 |
| 95 | 932 | CACAGUCGCUGGAUAGCUGAU | 95 | AUCAGCUAUCCAGCGACUGUGAG | 500 |
| 96 | 935 | AGUCGCUGGAUAGCUGAUCCU | 96 | AGGAUCAGCUAUCCAGCGACUGU | 501 |
| 97 | 938 | CGCUGGAUAGCUGAUCCUUCU | 97 | AGAAGGAUCAGCUAUCCAGCGAC | 502 |
| 98 | 954 | CUUCUCCUCAAAACAGUACAA | 98 | UUGUACUGUUUUGAGGAGAAGGA | 503 |
| 99 | 957 | CUCCUCAAAACAGUACAGCAA | 99 | UUGCUGUACUGUUUUGAGGAGAA | 504 |
| 100 | 966 | ACAGUACAGCAGAUACUUCUA | 100 | UAGAAGUAUCUGCUGUACUGUUU | 505 |
| 101 | 967 | CAGUACAGCAGAUACUUCUAA | 101 | UUAGAAGUAUCUGCUGUACUGUU | 506 |
| 102 | 968 | AGUACAGCAGAUACUUCUAAA | 102 | UUUAGAAGUAUCUGCUGUACUGU | 507 |
| 103 | 971 | ACAGCAGAUACUUCUAAGGUU | 103 | AACCUUAGAAGUAUCUGCUGUAC | 508 |
| 104 | 972 | CAGCAGAUACUUCUAAGGUUU | 104 | AAACCUUAGAAGUAUCUGCUGUA | 509 |
| 105 | 1063 | UAAAAUGAUCAGCAUGGAUAU | 105 | AUAUCCAUGCUGAUCAUUUUAGA | 510 |
| 106 | 1066 | AAUGAUCAGCAUGGAUAUUGA | 106 | UCAAUAUCCAUGCUGAUCAUUUU | 511 |
| 107 | 1070 | AUCAGCAUGGAUAUUGAACAA | 107 | UUGUUCAAUAUCCAUGCUGAUCA | 512 |
| 108 | 1076 | AUGGAUAUUGAACAAGUUAUU | 108 | AAUAACUUGUUCAAUAUCCAUGC | 513 |
| 109 | 1077 | UGGAUAUUGAACAAGUUAUUA | 109 | UAAUAACUUGUUCAAUAUCCAUG | 514 |
| 110 | 1082 | AUUGAACAAGUUAUUACCCUA | 110 | UAGGGUAAUAACUUGUUCAAUAU | 515 |
| 111 | 1083 | UUGAACAAGUUAUUACCCUAA | 111 | UUAGGGUAAUAACUUGUUCAAUA | 516 |
| 112 | 1085 | GAACAAGUUAUUACCCUAAGU | 112 | ACUUAGGGUAAUAACUUGUUCAA | 517 |
| 113 | 1086 | AACAAGUUAUUACCCUAAGUU | 113 | AACUUAGGGUAAUAACUUGUUCA | 518 |
| 114 | 1087 | ACAAGUUAUUACCCUAAGUUU | 114 | AAACUUAGGGUAAUAACUUGUUC | 519 |
| 115 | 1088 | CAAGUUAUUACCCUAAGUUUA | 115 | UAAACUUAGGGUAAUAACUUGUU | 520 |
| 116 | 1090 | AGUUAUUACCCUAAGUUUAGA | 116 | UCUAAACUUAGGGUAAUAACUUG | 521 |
| 117 | 1112 | CUCCUUCUGGCUGUCAAGUAA | 117 | UUACUUGACAGCCAGAAGGAGAG | 522 |
| 118 | 1117 | UCUGGCUGUCAAGUACAUCUU | 118 | AAGAUGUACUUGACAGCCAGAAG | 523 |
| 119 | 1124 | GUCAAGUACAUCUUCUUUGAA | 119 | UUCAAAGAAGAUGUACUUGACAG | 524 |
| 120 | 1126 | CAAGUACAUCUUCUUUGAACA | 120 | UGUUCAAAGAAGAUGUACUUGAC | 525 |
| 121 | 1127 | AAGUACAUCUUCUUUGAACAA | 121 | UUGUUCAAAGAAGAUGUACUUGA | 526 |
| 122 | 1149 | CAGAGACAGAAUCUACACUCU | 122 | AGAGUGUAGAUUCUGUCUCUGUU | 527 |
| 123 | 1172 | UUAAAAAACCCUAUCACAUCU | 123 | AGAUGUGAUAGGGUUUUUUAAUG | 528 |
| 124 | 1181 | CCUAUCACAUCUCCUGUAGUA | 124 | UACUACAGGAGAUGUGAUAGGGU | 529 |
| 125 | 1183 | UAUCACAUCUCCUGUAGUGAA | 125 | UUCACUACAGGAGAUGUGAUAGG | 530 |
| 126 | 1224 | AUUGUUGUAGACGUGAACCUA | 126 | UAGGUUCACGUCUACAACAAUUG | 531 |
| 127 | 1283 | GAAGAGACAGGGAUAAACCGA | 127 | UCGGUUUAUCCCUGUCUCUUCCU | 532 |
| 128 | 1284 | AAGAGACAGGGAUAAACCGAA | 128 | UUCGGUUUAUCCCUGUCUCUUCC | 533 |
| 129 | 1285 | AGAGACAGGGAUAAACCGAGA | 129 | UCUCGGUUUAUCCCUGUCUCUUC | 534 |
| 130 | 1286 | GAGACAGGGAUAAACCGAGAA | 130 | UUCUCGGUUUAUCCCUGUCUCUU | 535 |
| 131 | 1287 | AGACAGGGAUAAACCGAGAAA | 131 | UUUCUCGGUUUAUCCCUGUCUCU | 536 |
| 132 | 1288 | GACAGGGAUAAACCGAGAAAA | 132 | UUUUCUCGGUUUAUCCCUGUCUC | 537 |
| 133 | 1289 | ACAGGGAUAAACCGAGAAAGA | 133 | UCUUUCUCGGUUUAUCCCUGUCU | 538 |
| 134 | 1290 | CAGGGAUAAACCGAGAAAGAA | 134 | UUCUUUCUCGGUUUAUCCCUGUC | 539 |
| 135 | 1291 | AGGGAUAAACCGAGAAAGAAA | 135 | UUUCUUUCUCGGUUUAUCCCUGU | 540 |
| 136 | 1297 | AAACCGAGAAAGAAAAGUUGA | 136 | UCAACUUUUCUUUCUCGGUUUAU | 541 |
| 137 | 1313 | GUUGAGGUUAUAAAACCCUUA | 137 | UAAGGGUUUUAUAACCUCAACUU | 542 |
| 138 | 1318 | GGUUAUAAAACCCUUAGUGGA | 138 | UCCACUAAGGGUUUUAUAACCUC | 543 |
| 139 | 1326 | AACCCUUAGUGGCUGAAACAA | 139 | UUGUUUCAGCCACUAAGGGUUUU | 544 |
| 140 | 1327 | ACCCUUAGUGGCUGAAACAGA | 140 | UCUGUUUCAGCCACUAAGGGUUU | 545 |
| 141 | 1328 | CCCUUAGUGGCUGAAACAGAU | 141 | AUCUGUUUCAGCCACUAAGGGUU | 546 |
| 142 | 1329 | CCUUAGUGGCUGAAACAGAUA | 142 | UAUCUGUUUCAGCCACUAAGGGU | 547 |
| 143 | 1357 | CAGAGCUACAUUUGUGGUUGA | 143 | UCAACCACAAAUGUAGCUCUGUU | 548 |
| 144 | 1360 | AGCUACAUUUGUGGUUGGUAA | 144 | UUACCAACCACAAAUGUAGCUCU | 549 |
| 145 | 1380 | ACUCCUCCUUACUCGAUACUU | 145 | AAGUAUCGAGUAAGGAGGAGUUA | 550 |
| 146 | 1381 | CUCCUCCUUACUCGAUACUUA | 146 | UAAGUAUCGAGUAAGGAGGAGUU | 551 |
| 147 | 1410 | UGGUGACACAGGAACCUGAAA | 147 | UUUCAGGUUCCUGUGUCACCAGU | 552 |
| 148 | 1494 | AAGGUGCAAAAUUCCUUAGUA | 148 | UACUAAGGAAUUUUGCACCUUUC | 553 |
| 149 | 1505 | UUCCUUAGUGAUGCUGAGAUA | 149 | UAUCUCAGCAUCACUAAGGAAUU | 554 |
| 150 | 1510 | UAGUGAUGCUGAGAUCAUCCA | 150 | UGGAUGAUCUCAGCAUCACUAAG | 555 |
| 151 | 1514 | GAUGCUGAGAUCAUCCAGUUA | 151 | UAACUGGAUGAUCUCAGCAUCAC | 556 |
| 152 | 1516 | UGCUGAGAUCAUCCAGUUAGU | 152 | ACUAACUGGAUGAUCUCAGCAUC | 557 |
| 153 | 1519 | UGAGAUCAUCCAGUUAGUCAA | 153 | UUGACUAACUGGAUGAUCUCAGC | 558 |
| 154 | 1520 | GAGAUCAUCCAGUUAGUCAAU | 154 | AUUGACUAACUGGAUGAUCUCAG | 559 |
| 155 | 1521 | AGAUCAUCCAGUUAGUCAAUA | 155 | UAUUGACUAACUGGAUGAUCUCA | 560 |
| 156 | 1523 | AUCAUCCAGUUAGUCAAUGCU | 156 | AGCAUUGACUAACUGGAUGAUCU | 561 |
| 157 | 1525 | CAUCCAGUUAGUCAAUGCUAA | 157 | UUAGCAUUGACUAACUGGAUGAU | 562 |
| 158 | 1527 | UCCAGUUAGUCAAUGCUAAGA | 158 | UCUUAGCAUUGACUAACUGGAUG | 563 |
| 159 | 1528 | CCAGUUAGUCAAUGCUAAGCA | 159 | UGCUUAGCAUUGACUAACUGGAU | 564 |
| 160 | 1529 | CAGUUAGUCAAUGCUAAGCAU | 160 | AUGCUUAGCAUUGACUAACUGGA | 565 |
| 161 | 1530 | AGUUAGUCAAUGCUAAGCAUA | 161 | UAUGCUUAGCAUUGACUAACUGG | 566 |
| 162 | 1531 | GUUAGUCAAUGCUAAGCAUAU | 162 | AUAUGCUUAGCAUUGACUAACUG | 567 |
| 163 | 1532 | UUAGUCAAUGCUAAGCAUAUA | 163 | UAUAUGCUUAGCAUUGACUAACU | 568 |
| 164 | 1535 | GUCAAUGCUAAGCAUAUCCCA | 164 | UGGGAUAUGCUUAGCAUUGACUA | 569 |
| 165 | 1540 | UGCUAAGCAUAUCCCAGCCUA | 165 | UAGGCUGGGAUAUGCUUAGCAUU | 570 |
| 166 | 1543 | UAAGCAUAUCCCAGCCUACAA | 166 | UUGUAGGCUGGGAUAUGCUUAGC | 571 |
| 167 | 1546 | GCAUAUCCCAGCCUACAAGUU | 167 | AACUUGUAGGCUGGGAUAUGCUU | 572 |
| 168 | 1547 | CAUAUCCCAGCCUACAAGUUA | 168 | UAACUUGUAGGCUGGGAUAUGCU | 573 |
| 169 | 1548 | AUAUCCCAGCCUACAAGUUGA | 169 | UCAACUUGUAGGCUGGGAUAUGC | 574 |
| 170 | 1551 | UCCCAGCCUACAAGUUGGAAA | 170 | UUUCCAACUUGUAGGCUGGGAUA | 575 |
| 171 | 1555 | AGCCUACAAGUUGGAAACUCU | 171 | AGAGUUUCCAACUUGUAGGCUGG | 576 |
| 172 | 1558 | CUACAAGUUGGAAACUCUGAU | 172 | AUCAGAGUUUCCAACUUGUAGGC | 577 |
| 173 | 1561 | CAAGUUGGAAACUCUGAUGGA | 173 | UCCAUCAGAGUUUCCAACUUGUA | 578 |
| 174 | 1562 | AAGUUGGAAACUCUGAUGGAA | 174 | UUCCAUCAGAGUUUCCAACUUGU | 579 |
| 175 | 1567 | GGAAACUCUGAUGGAAACUCA | 175 | UGAGUUUCCAUCAGAGUUUCCAA | 580 |
| 176 | 1580 | GAAACUCAUGAGCGUGGUGUA | 176 | UACACCACGCUCAUGAGUUUCCA | 581 |
| 177 | 1582 | AACUCAUGAGCGUGGUGUAUA | 177 | UAUACACCACGCUCAUGAGUUUC | 582 |
| 178 | 1583 | ACUCAUGAGCGUGGUGUAUCU | 178 | AGAUACACCACGCUCAUGAGUUU | 583 |
| 179 | 1584 | CUCAUGAGCGUGGUGUAUCUA | 179 | UAGAUACACCACGCUCAUGAGUU | 584 |
| 180 | 1585 | UCAUGAGCGUGGUGUAUCUAU | 180 | AUAGAUACACCACGCUCAUGAGU | 585 |
| 181 | 1586 | CAUGAGCGUGGUGUAUCUAUU | 181 | AAUAGAUACACCACGCUCAUGAG | 586 |
| 182 | 1587 | AUGAGCGUGGUGUAUCUAUUA | 182 | UAAUAGAUACACCACGCUCAUGA | 587 |
| 183 | 1588 | UGAGCGUGGUGUAUCUAUUCA | 183 | UGAAUAGAUACACCACGCUCAUG | 588 |
| 184 | 1589 | GAGCGUGGUGUAUCUAUUCGA | 184 | UCGAAUAGAUACACCACGCUCAU | 589 |
| 185 | 1656 | ACCUACCUUACAGGGAUUAUA | 185 | UAUAAUCCCUGUAAGGUAGGUAC | 590 |
| 186 | 1657 | CCUACCUUACAGGGAUUAUAA | 186 | UUAUAAUCCCUGUAAGGUAGGUA | 591 |
| 187 | 1658 | CUACCUUACAGGGAUUAUAAU | 187 | AUUAUAAUCCCUGUAAGGUAGGU | 592 |
| 188 | 1664 | UACAGGGAUUAUAAUUACUCA | 188 | UGAGUAAUUAUAAUCCCUGUAAG | 593 |
| 189 | 1702 | UUGUGAGAAUGUUAUUGGAUA | 189 | UAUCCAAUAACAUUCUCACAACA | 594 |
| 190 | 1702 | UUGUGAGAAUGUUAUUGGAUA | 190 | UAUCCAAUAACAUUCUCACAACA | 595 |
| 191 | 1702 | UUGUGAGAAUGUUAUUGGAUA | 191 | UAUCCAAUAACAUUCUCACAACA | 596 |
| 192 | 1862 | AGCAGCCGAGUCCUUGCAGAU | 192 | AUCUGCAAGGACUCGGCUGCUGG | 597 |
| 193 | 1875 | UUGCAGAUGGGAUGACUCGUA | 193 | UACGAGUCAUCCCAUCUGCAAGG | 598 |
| 194 | 1876 | UGCAGAUGGGAUGACUCGUGA | 194 | UCACGAGUCAUCCCAUCUGCAAG | 599 |
| 195 | 1878 | CAGAUGGGAUGACUCGUGGCA | 195 | UGCCACGAGUCAUCCCAUCUGCA | 600 |
| 196 | 1879 | AGAUGGGAUGACUCGUGGCCA | 196 | UGGCCACGAGUCAUCCCAUCUGC | 601 |
| 197 | 1889 | ACUCGUGGCCCAGUUGUGCGU | 197 | ACGCACAACUGGGCCACGAGUCA | 602 |
| 198 | 1898 | CCAGUUGUGCGUCUUCCACGU | 198 | ACGUGGAAGACGCACAACUGGGC | 603 |
| 199 | 1901 | GUUGUGCGUCUUCCACGUGCU | 199 | AGCACGUGGAAGACGCACAACUG | 604 |
| 200 | 1935 | AAGUGAAAGCCUGGCUCGAAA | 200 | UUUCGAGCCAGGCUUUCACUUCU | 605 |
| 201 | 1936 | AGUGAAAGCCUGGCUCGAAAA | 201 | UUUUCGAGCCAGGCUUUCACUUC | 606 |
| 202 | 1943 | GCCUGGCUCGAAACAUCUGAA | 202 | UUCAGAUGUUUCGAGCCAGGCUU | 607 |
| 203 | 1945 | CUGGCUCGAAACAUCUGAAGA | 203 | UCUUCAGAUGUUUCGAGCCAGGC | 608 |
| 204 | 1952 | GAAACAUCUGAAGGGUUCGCA | 204 | UGCGAACCCUUCAGAUGUUUCGA | 609 |
| 205 | 1953 | AAACAUCUGAAGGGUUCGCAA | 205 | UUGCGAACCCUUCAGAUGUUUCG | 610 |
| 206 | 1958 | UCUGAAGGGUUCGCAGUGAUA | 206 | UAUCACUGCGAACCCUUCAGAUG | 611 |
| 207 | 1959 | CUGAAGGGUUCGCAGUGAUAA | 207 | UUAUCACUGCGAACCCUUCAGAU | 612 |
| 208 | 1960 | UGAAGGGUUCGCAGUGAUAAA | 208 | UUUAUCACUGCGAACCCUUCAGA | 613 |
| 209 | 1961 | GAAGGGUUCGCAGUGAUAAAA | 209 | UUUUAUCACUGCGAACCCUUCAG | 614 |
| 210 | 1963 | AGGGUUCGCAGUGAUAAAGGA | 210 | UCCUUUAUCACUGCGAACCCUUC | 615 |
| 211 | 1983 | AGGCAUUUGACAGCACUAGCA | 211 | UGCUAGUGCUGUCAAAUGCCUCC | 616 |
| 212 | 1985 | GCAUUUGACAGCACUAGCAGA | 212 | UCUGCUAGUGCUGUCAAAUGCCU | 617 |
| 213 | 1988 | UUUGACAGCACUAGCAGAUUU | 213 | AAAUCUGCUAGUGCUGUCAAAUG | 618 |
| 214 | 1989 | UUGACAGCACUAGCAGAUUUA | 214 | UAAAUCUGCUAGUGCUGUCAAAU | 619 |
| 215 | 1991 | GACAGCACUAGCAGAUUUGCA | 215 | UGCAAAUCUGCUAGUGCUGUCAA | 620 |
| 216 | 1995 | GCACUAGCAGAUUUGCACGUA | 216 | UACGUGCAAAUCUGCUAGUGCUG | 621 |
| 217 | 1996 | CACUAGCAGAUUUGCACGUCU | 217 | AGACGUGCAAAUCUGCUAGUGCU | 622 |
| 218 | 1998 | CUAGCAGAUUUGCACGUCUAA | 218 | UUAGACGUGCAAAUCUGCUAGUG | 623 |
| 219 | 1999 | UAGCAGAUUUGCACGUCUACA | 219 | UGUAGACGUGCAAAUCUGCUAGU | 624 |
| 220 | 2000 | AGCAGAUUUGCACGUCUACAA | 220 | UUGUAGACGUGCAAAUCUGCUAG | 625 |
| 221 | 2004 | GAUUUGCACGUCUACAGAAAA | 221 | UUUUCUGUAGACGUGCAAAUCUG | 626 |
| 222 | 2006 | UUUGCACGUCUACAGAAACUU | 222 | AAGUUUCUGUAGACGUGCAAAUC | 627 |
| 223 | 2007 | UUGCACGUCUACAGAAACUUA | 223 | UAAGUUUCUGUAGACGUGCAAAU | 628 |
| 224 | 2037 | UAGCUGGACGCAACCUUUAUA | 224 | UAUAAAGGUUGCGUCCAGCUAUA | 629 |
| 225 | 2040 | CUGGACGCAACCUUUAUAUCA | 225 | UGAUAUAAAGGUUGCGUCCAGCU | 630 |
| 226 | 2042 | GGACGCAACCUUUAUAUCCGU | 226 | ACGGAUAUAAAGGUUGCGUCCAG | 631 |
| 227 | 2043 | GACGCAACCUUUAUAUCCGUU | 227 | AACGGAUAUAAAGGUUGCGUCCA | 632 |
| 228 | 2044 | ACGCAACCUUUAUAUCCGUUU | 228 | AAACGGAUAUAAAGGUUGCGUCC | 633 |
| 229 | 2045 | CGCAACCUUUAUAUCCGUUUA | 229 | UAAACGGAUAUAAAGGUUGCGUC | 634 |
| 230 | 2047 | CAACCUUUAUAUCCGUUUCCA | 230 | UGGAAACGGAUAUAAAGGUUGCG | 635 |
| 231 | 2100 | UGAUUUCAAAGGGUACAGAGA | 231 | UCUCUGUACCCUUUGAAAUCAUG | 636 |
| 232 | 2169 | CCGUUAGUGGUAACUAUUGUA | 232 | UACAAUAGUUACCACUAACGGCU | 637 |
| 233 | 2170 | CGUUAGUGGUAACUAUUGUAA | 233 | UUACAAUAGUUACCACUAACGGC | 638 |
| 234 | 2172 | UUAGUGGUAACUAUUGUACUA | 234 | UAGUACAAUAGUUACCACUAACG | 639 |
| 235 | 2175 | GUGGUAACUAUUGUACUGACA | 235 | UGUCAGUACAAUAGUUACCACUA | 640 |
| 236 | 2176 | UGGUAACUAUUGUACUGACAA | 236 | UUGUCAGUACAAUAGUUACCACU | 641 |
| 237 | 2179 | UAACUAUUGUACUGACAAGAA | 237 | UUCUUGUCAGUACAAUAGUUACC | 642 |
| 238 | 2183 | UAUUGUACUGACAAGAAACCU | 238 | AGGUUUCUUGUCAGUACAAUAGU | 643 |
| 239 | 2193 | ACAAGAAACCUGCUGCUAUAA | 239 | UUAUAGCAGCAGGUUUCUUGUCA | 644 |
| 240 | 2240 | GUUGUUUGUGAAGCUGUCAUU | 240 | AAUGACAGCUUCACAAACAACAG | 645 |
| 241 | 2264 | GCCAAGGUUGUCAGAGAAGUA | 241 | UACUUCUCUGACAACCUUGGCUG | 646 |
| 242 | 2266 | CAAGGUUGUCAGAGAAGUAUU | 242 | AAUACUUCUCUGACAACCUUGGC | 647 |
| 243 | 2268 | AGGUUGUCAGAGAAGUAUUAA | 243 | UUAAUACUUCUCUGACAACCUUG | 648 |
| 244 | 2269 | GGUUGUCAGAGAAGUAUUAAA | 244 | UUUAAUACUUCUCUGACAACCUU | 649 |
| 245 | 2271 | UUGUCAGAGAAGUAUUAAAGA | 245 | UCUUUAAUACUUCUCUGACAACC | 650 |
| 246 | 2274 | UCAGAGAAGUAUUAAAGACUA | 246 | UAGUCUUUAAUACUUCUCUGACA | 651 |
| 247 | 2276 | AGAGAAGUAUUAAAGACUACA | 247 | UGUAGUCUUUAAUACUUCUCUGA | 652 |
| 248 | 2277 | GAGAAGUAUUAAAGACUACCA | 248 | UGGUAGUCUUUAAUACUUCUCUG | 653 |
| 249 | 2278 | AGAAGUAUUAAAGACUACCAA | 249 | UUGGUAGUCUUUAAUACUUCUCU | 654 |
| 250 | 2300 | GAGGCUAUGAUUGAGGUCAAA | 250 | UUUGACCUCAAUCAUAGCCUCUG | 655 |
| 251 | 2301 | AGGCUAUGAUUGAGGUCAACA | 251 | UGUUGACCUCAAUCAUAGCCUCU | 656 |
| 252 | 2302 | GGCUAUGAUUGAGGUCAACAU | 252 | AUGUUGACCUCAAUCAUAGCCUC | 657 |
| 253 | 2303 | GCUAUGAUUGAGGUCAACAUU | 253 | AAUGUUGACCUCAAUCAUAGCCU | 658 |
| 254 | 2305 | UAUGAUUGAGGUCAACAUUAA | 254 | UUAAUGUUGACCUCAAUCAUAGC | 659 |
| 255 | 2306 | AUGAUUGAGGUCAACAUUAAA | 255 | UUUAAUGUUGACCUCAAUCAUAG | 660 |
| 256 | 2310 | UUGAGGUCAACAUUAACAAGA | 256 | UCUUGUUAAUGUUGACCUCAAUC | 661 |
| 257 | 2311 | UGAGGUCAACAUUAACAAGAA | 257 | UUCUUGUUAAUGUUGACCUCAAU | 662 |
| 258 | 2317 | CAACAUUAACAAGAAUUUAGU | 258 | ACUAAAUUCUUGUUAAUGUUGAC | 663 |
| 259 | 2320 | CAUUAACAAGAAUUUAGUGGA | 259 | UCCACUAAAUUCUUGUUAAUGUU | 664 |
| 260 | 2323 | UAACAAGAAUUUAGUGGGCUA | 260 | UAGCCCACUAAAUUCUUGUUAAU | 665 |
| 261 | 2328 | AGAAUUUAGUGGGCUCUGCCA | 261 | UGGCAGAGCCCACUAAAUUCUUG | 666 |
| 262 | 2344 | UGCCAUGGCUGGGAGCAUAGA | 262 | UCUAUGCUCCCAGCCAUGGCAGA | 667 |
| 263 | 2345 | GCCAUGGCUGGGAGCAUAGGA | 263 | UCCUAUGCUCCCAGCCAUGGCAG | 668 |
| 264 | 2353 | UGGGAGCAUAGGAGGCUACAA | 264 | UUGUAGCCUCCUAUGCUCCCAGC | 669 |
| 265 | 2357 | AGCAUAGGAGGCUACAACGCA | 265 | UGCGUUGUAGCCUCCUAUGCUCC | 670 |
| 266 | 2358 | GCAUAGGAGGCUACAACGCCA | 266 | UGGCGUUGUAGCCUCCUAUGCUC | 671 |
| 267 | 2362 | AGGAGGCUACAACGCCCAUGA | 267 | UCAUGGGCGUUGUAGCCUCCUAU | 672 |
| 268 | 2368 | CUACAACGCCCAUGCAGCAAA | 268 | UUUGCUGCAUGGGCGUUGUAGCC | 673 |
| 269 | 2370 | ACAACGCCCAUGCAGCAAACA | 269 | UGUUUGCUGCAUGGGCGUUGUAG | 674 |
| 270 | 2376 | CCCAUGCAGCAAACAUUGUCA | 270 | UGACAAUGUUUGCUGCAUGGGCG | 675 |
| 271 | 2377 | CCAUGCAGCAAACAUUGUCAA | 271 | UUGACAAUGUUUGCUGCAUGGGC | 676 |
| 272 | 2399 | GCCAUCUACAUUGCCUGUGGA | 272 | UCCACAGGCAAUGUAGAUGGCGG | 677 |
| 273 | 2419 | ACAGGAUGCAGCACAGAAUGU | 273 | ACAUUCUGUGCUGCAUCCUGUCC | 678 |
| 274 | 2420 | CAGGAUGCAGCACAGAAUGUU | 274 | AACAUUCUGUGCUGCAUCCUGUC | 679 |
| 275 | 2424 | AUGCAGCACAGAAUGUUGGUA | 275 | UACCAACAUUCUGUGCUGCAUCC | 680 |
| 276 | 2426 | GCAGCACAGAAUGUUGGUAGU | 276 | ACUACCAACAUUCUGUGCUGCAU | 681 |
| 277 | 2429 | GCACAGAAUGUUGGUAGUUCA | 277 | UGAACUACCAACAUUCUGUGCUG | 682 |
| 278 | 2479 | UCCCACAAAUGAAGAUUUAUA | 278 | UAUAAAUCUUCAUUUGUGGGACC | 683 |
| 279 | 2482 | CACAAAUGAAGAUUUAUAUAU | 279 | AUAUAUAAAUCUUCAUUUGUGGG | 684 |
| 280 | 2484 | CAAAUGAAGAUUUAUAUAUCA | 280 | UGAUAUAUAAAUCUUCAUUUGUG | 685 |
| 281 | 2502 | UCAGCUGCACCAUGCCAUCUA | 281 | UAGAUGGCAUGGUGCAGCUGAUA | 686 |
| 282 | 2514 | UGCCAUCUAUAGAGAUAGGAA | 282 | UUCCUAUCUCUAUAGAUGGCAUG | 687 |
| 283 | 2575 | UUUGCAGAUGCUAGGUGUUCA | 283 | UGAACACCUAGCAUCUGCAAACA | 688 |
| 284 | 2576 | UUGCAGAUGCUAGGUGUUCAA | 284 | UUGAACACCUAGCAUCUGCAAAC | 689 |
| 285 | 2579 | CAGAUGCUAGGUGUUCAAGGA | 285 | UCCUUGAACACCUAGCAUCUGCA | 690 |
| 286 | 2587 | AGGUGUUCAAGGAGCAUGCAA | 286 | UUGCAUGCUCCUUGAACACCUAG | 691 |
| 287 | 2588 | GGUGUUCAAGGAGCAUGCAAA | 287 | UUUGCAUGCUCCUUGAACACCUA | 692 |
| 288 | 2592 | UUCAAGGAGCAUGCAAAGAUA | 288 | UAUCUUUGCAUGCUCCUUGAACA | 693 |
| 289 | 2593 | UCAAGGAGCAUGCAAAGAUAA | 289 | UUAUCUUUGCAUGCUCCUUGAAC | 694 |
| 290 | 2594 | CAAGGAGCAUGCAAAGAUAAU | 290 | AUUAUCUUUGCAUGCUCCUUGAA | 695 |
| 291 | 2606 | AAAGAUAAUCCUGGGGAAAAU | 291 | AUUUUCCCCAGGAUUAUCUUUGC | 696 |
| 292 | 2620 | GGAAAAUGCCCGGCAGCUUGA | 292 | UCAAGCUGCCGGGCAUUUUCCCC | 697 |
| 293 | 2627 | GCCCGGCAGCUUGCCCGAAUU | 293 | AAUUCGGGCAAGCUGCCGGGCAU | 698 |
| 294 | 2633 | CAGCUUGCCCGAAUUGUGUGU | 294 | ACACACAAUUCGGGCAAGCUGCC | 699 |
| 295 | 2658 | CCGUAAUGGCUGGGGAAUUGU | 295 | ACAAUUCCCCAGCCAUUACGGUC | 700 |
| 296 | 2674 | AUUGUCACUUAUGGCAGCAUU | 296 | AAUGCUGCCAUAAGUGACAAUUC | 701 |
| 297 | 2677 | GUCACUUAUGGCAGCAUUGGA | 297 | UCCAAUGCUGCCAUAAGUGACAA | 702 |
| 298 | 2721 | ACAUGAUUCACAACAGGUCGA | 298 | UCGACCUGUUGUGAAUCAUGUGA | 703 |
| 299 | 2722 | CAUGAUUCACAACAGGUCGAA | 299 | UUCGACCUGUUGUGAAUCAUGUG | 704 |
| 300 | 2723 | AUGAUUCACAACAGGUCGAAA | 300 | UUUCGACCUGUUGUGAAUCAUGU | 705 |
| 301 | 2724 | UGAUUCACAACAGGUCGAAGA | 301 | UCUUCGACCUGUUGUGAAUCAUG | 706 |
| 302 | 2725 | GAUUCACAACAGGUCGAAGAU | 302 | AUCUUCGACCUGUUGUGAAUCAU | 707 |
| 303 | 2727 | UUCACAACAGGUCGAAGAUCA | 303 | UGAUCUUCGACCUGUUGUGAAUC | 708 |
| 304 | 2728 | UCACAACAGGUCGAAGAUCAA | 304 | UUGAUCUUCGACCUGUUGUGAAU | 709 |
| 305 | 2729 | CACAACAGGUCGAAGAUCAAU | 305 | AUUGAUCUUCGACCUGUUGUGAA | 710 |
| 306 | 2731 | CAACAGGUCGAAGAUCAAUUU | 306 | AAAUUGAUCUUCGACCUGUUGUG | 711 |
| 307 | 2732 | AACAGGUCGAAGAUCAAUUUA | 307 | UAAAUUGAUCUUCGACCUGUUGU | 712 |
| 308 | 2734 | CAGGUCGAAGAUCAAUUUACA | 308 | UGUAAAUUGAUCUUCGACCUGUU | 713 |
| 309 | 2735 | AGGUCGAAGAUCAAUUUACAA | 309 | UUGUAAAUUGAUCUUCGACCUGU | 714 |
| 310 | 2736 | GGUCGAAGAUCAAUUUACAAA | 310 | UUUGUAAAUUGAUCUUCGACCUG | 715 |
| 311 | 2737 | GUCGAAGAUCAAUUUACAAGA | 311 | UCUUGUAAAUUGAUCUUCGACCU | 716 |
| 312 | 2738 | UCGAAGAUCAAUUUACAAGAA | 312 | UUCUUGUAAAUUGAUCUUCGACC | 717 |
| 313 | 2740 | GAAGAUCAAUUUACAAGACCU | 313 | AGGUCUUGUAAAUUGAUCUUCGA | 718 |
| 314 | 2741 | AAGAUCAAUUUACAAGACCUA | 314 | UAGGUCUUGUAAAUUGAUCUUCG | 719 |
| 315 | 2742 | AGAUCAAUUUACAAGACCUCA | 315 | UGAGGUCUUGUAAAUUGAUCUUC | 720 |
| 316 | 2743 | GAUCAAUUUACAAGACCUCCA | 316 | UGGAGGUCUUGUAAAUUGAUCUU | 721 |
| 317 | 2744 | AUCAAUUUACAAGACCUCCAA | 317 | UUGGAGGUCUUGUAAAUUGAUCU | 722 |
| 318 | 2833 | UAAAGGACUAACAUAAAAUCU | 318 | AGAUUUUAUGUUAGUCCUUUAGA | 723 |
| 319 | 2834 | AAAGGACUAACAUAAAAUCUA | 319 | UAGAUUUUAUGUUAGUCCUUUAG | 724 |
| 320 | 2937 | UCAGAGAGGUCUCAGGUUCUU | 320 | AAGAACCUGAGACCUCUCUGAAA | 725 |
| 321 | 2941 | AGAGGUCUCAGGUUCUUUCCA | 321 | UGGAAAGAACCUGAGACCUCUCU | 726 |
| 322 | 2945 | GUCUCAGGUUCUUUCCAUGCA | 322 | UGCAUGGAAAGAACCUGAGACCU | 727 |
| 323 | 2947 | CUCAGGUUCUUUCCAUGCAGA | 323 | UCUGCAUGGAAAGAACCUGAGAC | 728 |
| 324 | 2981 | AACACAGUUUAGUGCUUUACA | 324 | UGUAAAGCACUAAACUGUGUUCA | 729 |
| 325 | 2994 | GCUUUACAUGCUGUGCUCUUU | 325 | AAAGAGCACAGCAUGUAAAGCAC | 730 |
| 326 | 3058 | UGGUAAUCUACAGCUCACCUA | 326 | UAGGUGAGCUGUAGAUUACCAUC | 731 |
| 327 | 3068 | CAGCUCACCUCUGAAGGCAAA | 327 | UUUGCCUUCAGAGGUGAGCUGUA | 732 |
| 328 | 3071 | CUCACCUCUGAAGGCAAAUAU | 328 | AUAUUUGCCUUCAGAGGUGAGCU | 733 |
| 329 | 3102 | AAAAGUUUUGAUGAAAUUCUU | 329 | AAGAAUUUCAUCAAAACUUUUUU | 734 |
| 330 | 3105 | AGUUUUGAUGAAAUUCUUGAA | 330 | UUCAAGAAUUUCAUCAAAACUUU | 735 |
| 331 | 3108 | UUUGAUGAAAUUCUUGAAGUU | 331 | AACUUCAAGAAUUUCAUCAAAAC | 736 |
| 332 | 3110 | UGAUGAAAUUCUUGAAGUUCA | 332 | UGAACUUCAAGAAUUUCAUCAAA | 737 |
| 333 | 3111 | GAUGAAAUUCUUGAAGUUCAU | 333 | AUGAACUUCAAGAAUUUCAUCAA | 738 |
| 334 | 3117 | AUUCUUGAAGUUCAUGGUGAU | 334 | AUCACCAUGAACUUCAAGAAUUU | 739 |
| 335 | 3126 | GUUCAUGGUGAUCAGUGCAAU | 335 | AUUGCACUGAUCACCAUGAACUU | 740 |
| 336 | 3129 | CAUGGUGAUCAGUGCAAUUGA | 336 | UCAAUUGCACUGAUCACCAUGAA | 741 |
| 337 | 3130 | AUGGUGAUCAGUGCAAUUGAA | 337 | UUCAAUUGCACUGAUCACCAUGA | 742 |
| 338 | 3207 | GAAACUCCUGAUUUUGUAGUU | 338 | AACUACAAAAUCAGGAGUUUCAU | 743 |
| 339 | 3208 | AAACUCCUGAUUUUGUAGUUA | 339 | UAACUACAAAAUCAGGAGUUUCA | 744 |
| 340 | 3209 | AACUCCUGAUUUUGUAGUUAA | 340 | UUAACUACAAAAUCAGGAGUUUC | 745 |
| 341 | 3210 | ACUCCUGAUUUUGUAGUUAAU | 341 | AUUAACUACAAAAUCAGGAGUUU | 746 |
| 342 | 3212 | UCCUGAUUUUGUAGUUAAUUU | 342 | AAAUUAACUACAAAAUCAGGAGU | 747 |
| 343 | 3213 | CCUGAUUUUGUAGUUAAUUUA | 343 | UAAAUUAACUACAAAAUCAGGAG | 748 |
| 344 | 3229 | AUUUAUUAAGUCUGGGAUGUA | 344 | UACAUCCCAGACUUAAUAAAUUA | 749 |
| 345 | 3230 | UUUAUUAAGUCUGGGAUGUAA | 345 | UUACAUCCCAGACUUAAUAAAUU | 750 |
| 346 | 3241 | UGGGAUGUAGAACUUCAAGAA | 346 | UUCUUGAAGUUCUACAUCCCAGA | 751 |
| 347 | 3243 | GGAUGUAGAACUUCAAGAAGU | 347 | ACUUCUUGAAGUUCUACAUCCCA | 752 |
| 348 | 3244 | GAUGUAGAACUUCAAGAAGUA | 348 | UACUUCUUGAAGUUCUACAUCCC | 753 |
| 349 | 3252 | ACUUCAAGAAGUAAGAGCUAA | 349 | UUAGCUCUUACUUCUUGAAGUUC | 754 |
| 350 | 3254 | UUCAAGAAGUAAGAGCUAAGU | 350 | ACUUAGCUCUUACUUCUUGAAGU | 755 |
| 351 | 3256 | CAAGAAGUAAGAGCUAAGUUA | 351 | UAACUUAGCUCUUACUUCUUGAA | 756 |
| 352 | 3332 | GGGGGUAAUCAGCAUUAUUCU | 352 | AGAAUAAUGCUGAUUACCCCCCA | 757 |
| 353 | 3333 | GGGGUAAUCAGCAUUAUUCUU | 353 | AAGAAUAAUGCUGAUUACCCCCC | 758 |
| 354 | 3400 | AAACUACAGAAUAAUGUGUUA | 354 | UAACACAUUAUUCUGUAGUUUGG | 759 |
| 355 | 3401 | AACUACAGAAUAAUGUGUUAA | 355 | UUAACACAUUAUUCUGUAGUUUG | 760 |
| 356 | 3402 | ACUACAGAAUAAUGUGUUAAA | 356 | UUUAACACAUUAUUCUGUAGUUU | 761 |
| 357 | 3460 | AUUUAUCUCCGCAGGCUAUUU | 357 | AAAUAGCCUGCGGAGAUAAAUAC | 762 |
| 358 | 3465 | UCUCCGCAGGCUAUUUGUUCA | 358 | UGAACAAAUAGCCUGCGGAGAUA | 763 |
| 359 | 3466 | CUCCGCAGGCUAUUUGUUCAA | 359 | UUGAACAAAUAGCCUGCGGAGAU | 764 |
| 360 | 3482 | UUCAGAGAGGCCUUUUGUUUA | 360 | UAAACAAAAGGCCUCUCUGAACA | 765 |
| 361 | 3483 | UCAGAGAGGCCUUUUGUUUAA | 361 | UUAAACAAAAGGCCUCUCUGAAC | 766 |
| 362 | 3484 | CAGAGAGGCCUUUUGUUUAAA | 362 | UUUAAACAAAAGGCCUCUCUGAA | 767 |
| 363 | 3485 | AGAGAGGCCUUUUGUUUAAAU | 363 | AUUUAAACAAAAGGCCUCUCUGA | 768 |
| 364 | 3487 | AGAGGCCUUUUGUUUAAAUAU | 364 | AUAUUUAAACAAAAGGCCUCUCU | 769 |
| 365 | 3532 | CUGGAUUGGCUAUAACAUGUA | 365 | UACAUGUUAUAGCCAAUCCAGAC | 770 |
| 366 | 3533 | UGGAUUGGCUAUAACAUGUCU | 366 | AGACAUGUUAUAGCCAAUCCAGA | 771 |
| 367 | 3537 | UUGGCUAUAACAUGUCUUUCA | 367 | UGAAAGACAUGUUAUAGCCAAUC | 772 |
| 368 | 3550 | GUCUUUCAGCAUUAGGCUUUU | 368 | AAAAGCCUAAUGCUGAAAGACAU | 773 |
| 369 | 3597 | ACUAAAGAUAUCAGAGCUCUU | 369 | AAGAGCUCUGAUAUCUUUAGUAA | 774 |
| 370 | 3598 | CUAAAGAUAUCAGAGCUCUUA | 370 | UAAGAGCUCUGAUAUCUUUAGUA | 775 |
| 371 | 3651 | AAGCAAGACUGGGACCUUAGA | 371 | UCUAAGGUCCCAGUCUUGCUUGU | 776 |
| 372 | 3652 | AGCAAGACUGGGACCUUAGAA | 372 | UUCUAAGGUCCCAGUCUUGCUUG | 777 |
| 373 | 3654 | CAAGACUGGGACCUUAGAAAU | 373 | AUUUCUAAGGUCCCAGUCUUGCU | 778 |
| 374 | 3655 | AAGACUGGGACCUUAGAAAUA | 374 | UAUUUCUAAGGUCCCAGUCUUGC | 779 |
| 375 | 3656 | AGACUGGGACCUUAGAAAUCA | 375 | UGAUUUCUAAGGUCCCAGUCUUG | 780 |
| 376 | 3714 | UCUAAGCCAACUUUAAUUGCU | 376 | AGCAAUUAAAGUUGGCUUAGAGA | 781 |
| 377 | 3770 | UUUUUUGUAAACUGUAUCAAA | 377 | UUUGAUACAGUUUACAAAAAAAA | 782 |
| 378 | 3773 | UUUGUAAACUGUAUCAAAUCU | 378 | AGAUUUGAUACAGUUUACAAAAA | 783 |
| 379 | 3784 | UAUCAAAUCUGUAUAUGUUGU | 379 | ACAACAUAUACAGAUUUGAUACA | 784 |
| 380 | 3786 | UCAAAUCUGUAUAUGUUGUAA | 380 | UUACAACAUAUACAGAUUUGAUA | 785 |
| 381 | 3788 | AAAUCUGUAUAUGUUGUAAUA | 381 | UAUUACAACAUAUACAGAUUUGA | 786 |
| 382 | 3789 | AAUCUGUAUAUGUUGUAAUAA | 382 | UUAUUACAACAUAUACAGAUUUG | 787 |
| 383 | 3790 | AUCUGUAUAUGUUGUAAUAAA | 383 | UUUAUUACAACAUAUACAGAUUU | 788 |
| 384 | 3814 | UAUGCUAGUUUAUUGGAAGUA | 384 | UACUUCCAAUAAACUAGCAUAAG | 789 |
| 385 | 3816 | UGCUAGUUUAUUGGAAGUGUU | 385 | AACACUUCCAAUAAACUAGCAUA | 790 |
| 386 | 3825 | AUUGGAAGUGUUCAAGAAAUA | 386 | UAUUUCUUGAACACUUCCAAUAA | 791 |
| 387 | 3826 | UUGGAAGUGUUCAAGAAAUAA | 387 | UUAUUUCUUGAACACUUCCAAUA | 792 |
| 388 | 3827 | UGGAAGUGUUCAAGAAAUAAA | 388 | UUUAUUUCUUGAACACUUCCAAU | 793 |
| 389 | 3850 | UCAACUUGUGUACUGAUAAAA | 389 | UUUUAUCAGUACACAAGUUGAUU | 794 |
| 390 | 4097 | GUCAGCAGAGUUAUUGAAUCU | 390 | AGAUUCAAUAACUCUGCUGACCC | 795 |
| 391 | 4099 | CAGCAGAGUUAUUGAAUCUUA | 391 | UAAGAUUCAAUAACUCUGCUGAC | 796 |
| 392 | 4100 | AGCAGAGUUAUUGAAUCUUAA | 392 | UUAAGAUUCAAUAACUCUGCUGA | 797 |
| 393 | 4102 | CAGAGUUAUUGAAUCUUAAUU | 393 | AAUUAAGAUUCAAUAACUCUGCU | 798 |
| 394 | 4103 | AGAGUUAUUGAAUCUUAAUUU | 394 | AAAUUAAGAUUCAAUAACUCUGC | 799 |
| 395 | 4120 | AUUUUUUUUAAUGUACAAGUU | 395 | AACUUGUACAUUAAAAAAAAUUA | 800 |
| 396 | 4154 | AAAGAACUCCUUAUUUUGUAU | 396 | AUACAAAAUAAGGAGUUCUUUAU | 801 |
| 397 | 4459 | UAAUGUUUUGUACAAUUACUA | 397 | UAGUAAUUGUACAAAACAUUAAU | 802 |
| 398 | 4480 | AAUUGUAUACAUUUUGUUAUA | 398 | UAUAACAAAAUGUAUACAAUUUA | 803 |
| 399 | 4482 | UUGUAUACAUUUUGUUAUAGA | 399 | UCUAUAACAAAAUGUAUACAAUU | 804 |
| 400 | 4483 | UGUAUACAUUUUGUUAUAGAA | 400 | UUCUAUAACAAAAUGUAUACAAU | 805 |
| 401 | 4484 | GUAUACAUUUUGUUAUAGAAU | 401 | AUUCUAUAACAAAAUGUAUACAA | 806 |
| 402 | 4485 | UAUACAUUUUGUUAUAGAAUA | 402 | UAUUCUAUAACAAAAUGUAUACA | 807 |
| 403 | 4486 | AUACAUUUUGUUAUAGAAUAA | 403 | UUAUUCUAUAACAAAAUGUAUAC | 808 |
| 404 | 4488 | ACAUUUUGUUAUAGAAUACUU | 404 | AAGUAUUCUAUAACAAAAUGUAU | 809 |
| 405 | 4496 | UUAUAGAAUACUUUUUUCUAA | 405 | UUAGAAAAAAGUAUUCUAUAACA | 810 |
| 702 | 110 | GAUUCUGUAGCUACAAUGUUA | 1434 | UAACAUUGUAGCUACAGAAUCCU | 1441 |
| 703 | 111 | AUUCUGUAGCUACAAUGUUGU | 1435 | ACAACAUUGUAGCUACAGAAUCC | 1442 |
| 704 | 115 | UGUAGCUACAAUGUUGUCAAA | 1436 | UUUGACAACAUUGUAGCUACAGA | 1443 |
| 705 | 2843 | ACAUAAAAUCUGUGAAUUAAA | 1437 | UUUAAUUCACAGAUUUUAUGUUA | 1444 |
| 706 | 2835 | AAGGACUAACAUAAAAUCUGU | 1438 | ACAGAUUUUAUGUUAGUCCUUUA | 1445 |
| 707 | 3277 | UAAGUUCAUGUUUGUAAAUUA | 1439 | UAAUUUACAAACAUGAACUUAGA | 1446 |
| 708 | 3418 | UUAAACAUGCUAAAUAGUUCU | 1440 | AGAACUAUUUAGCAUGUUUAACA | 1447 |

Homo sapiens HMGCR, transcript variant 1, mRNA: GenBank: NM_000859.3 (SEQ ID NO: 811)

| | |
|---|---|
| 1 | ccttccgctc cgcgactgcg ttaactggag ccaggctgag cgtcggcgcc ggggttcggt |
| 61 | ggcctctagt gagatctgga ggatccaagg attctgtagc tacaatgttg tcaagacttt |
| 121 | ttcgaatgca tggcctcttt gtggcctccc atccctggga agtcatagtg gggacagtga |
| 181 | cactgaccat ctgcatgatg tccatgaaca tgtttactgg taacaataag atctgtggtt |
| 241 | ggaattatga atgtccaaag tttgaagagg atgttttgag cagtgacatt ataattctga |
| 301 | caataacacg atgcatagcc atcctgtata tttacttcca gttccagaat ttacgtcaac |
| 361 | ttggatcaaa atatattttg ggtattgctg gccttttcac aattttctca agttttgtat |
| 421 | tcagtacagt tgtcattcac ttcttagaca aagaattgac aggcttgaat gaagctttgc |
| 481 | cctttttcct acttttgatt gacctttoca gagcaagcac attagcaaag tttgccctca |
| 541 | gttccaactc acaggatgaa gtaagggaaa atattgctcg tggaatggca attttaggtc |
| 601 | ctacgtttac cctcgatgct cttgttgaat gtcttgtgat tggagttggt accatgtcag |
| 661 | gggtacgtca gcttgaaatt atgtgctgct ttggctgcat gtcagttctt gccaactact |
| 721 | tcgtgttcat gactttcttc ccagcttgtg tgtccttggt attagagctt tctcgggaaa |
| 781 | gccgcgaggg togtccaatt tggcagctca gccattttgc ccgagtttta gaagaagaag |
| 841 | aaaataagcc gaatcctgta actcagaggg tcaagatgat tatgtctcta ggcttggttc |
| 901 | ttgttcatgc tcacagtcgc tggatagctg atccttctcc tcaaaacagt acagcagata |
| 961 | cttctaaggt ttcattagga ctggatgaaa atgtgtccaa gagaattgaa ccaagtgttt |
| 1021 | ccctctggca gttttatctc tctaaaatga tcagcatgga tattgaacaa gttattaccc |
| 1081 | taagtttagc tctccttctg gctgtcaagt acatcttctt tgaacaaaca gagacagaat |
| 1141 | ctacactctc attaaaaaac cctatcacat ctcctgtagt gacacaaaag aaagtcccag |
| 1201 | acaattgttg tagacgtgaa cctatgctgg tcagaaataa ccagaaatgt gattcagtag |
| 1261 | aggaagagac agggataaac cgagaaagaa aagttgaggt tataaaacce ttagtggctg |
| 1321 | aaacagatac cccaaacaga gctacatttg tggttggtaa ctcctcctta ctcgatactt |
| 1381 | catcagtact ggtgacacag gaacctgaaa ttgaacttcc cagggaacct cggcctaatg |
| 1441 | aagaatgtct acagatactt gggaatgcag agaaaggtgc aaaattcctt agtgatgctg |
| 1501 | agatcatcca gttagtcaat gctaagcata toccagccta caagttggaa actctgatgg |
| 1561 | aaactcatga gcgtggtgta tctattcgcc gacagttact ttccaagaag ctttcagaac |
| 1621 | cttcttctct ccagtaccta ccttacaggg attataatta ctccttggtg atgggagctt |
| 1681 | gttgtgagaa tgttattgga tatatgccca tccctgttgg agtggcagga cccctttgct |
| 1741 | tagatgaaaa agaatttcag gttccaatgg caacaacaga aggttgtctt gtggccagca |
| 1801 | ccaatagagg ctgcagagca ataggtcttg gtggaggtgc cagcagccga gtccttgcag |
| 1861 | atgggatgac tegtggccca gttgtgcgtc ttccacgtgc ttgtgactct gcagaagtga |
| 1921 | aagcctggct cgaaacatct gaagggttcg cagtgataaa ggaggcattt gacagcacta |
| 1981 | gcagatttgc acgtctacag aaacttcata caagtatage tggacgcaac ctttatatcc |
| 2041 | gtttccagtc caggtcaggg gatgccatgg ggatgaacat gatttcaaag ggtacagaga |
| 2101 | aagcactttc aaaacttcac gagtatttcc ctgaaatgca gattctagcc gttagtggta |
| 2161 | actattgtac tgacaagaaa cctgctgcta taaattggat agagggaaga ggaaaatctg |
| 2221 | ttgtttgtga agctgtcatt ccagccaagg ttgtcagaga agtattaaag actaccacag |
| 2281 | aggctatgat tgaggtcaac attaacaaga atttagtggg ctctgccatg gctgggagca |
| 2341 | taggaggcta caacgcccat gcagcaaaca ttgtcaccgc catctacatt gcctgtggac |
| 2401 | aggatgcagc acagaatgtt ggtagttcaa actgtattac tttaatggaa gcaagtggtc |
| 2461 | ccacaaatga agatttatat atcagctgca ccatgccatc tatagagata ggaacggtgg |
| 2521 | gtggtgggac caacctacta cctcagcaag cctgtttgca gatgctaggt gttcaaggag |
| 2581 | catgcaaaga taatcctggg gaaaatgccc ggcagcttgc ccgaattgtg tgtgggaccg |
| 2641 | taatggctgg ggaattgtca cttatggcag cattggcage aggacatctt gtcaaaagtc |
| 2701 | acatgattca caacaggtcg aagatcaatt tacaagacct ccaaggagct tgcaccaaga |
| 2761 | agacagcctg aatagcccga cagttctgaa ctggaacatg ggcattgggt tctaaaggac |
| 2821 | taacataaaa tctgtgaatt aaaaaagctc aatgcattgt cttgtggagg atgaatagat |
| 2881 | gtgatcactg agacagccac ttggtttttg gctctttcag agaggtctca ggttctttcc |
| 2941 | atgcagactc ctcagatctg aacacagttt agtgctttac atgctgtgct ctttgaagag |
| 3001 | atttcaacaa gaatattgta tgttaaagca tcagagatgg taatctacag ctcacctctg |
| 3061 | aaggcaaata taagctggga aaaaagtttt gatgaaattc ttgaagttca tggtgatcag |
| 3121 | tgcaattgac cttctccctc actcctgcca gttgaaaatg gatttttaaa ttatactgta |
| 3181 | gctgatgaaa ctcctgattt tgtagttaat ttattaagtc tgggatgtag aacttcaaga |
| 3241 | agtaagagct aagttctaag ttcatgtttg taaattaata cttcatttgg tgctggtcta |
| 3301 | ttttgatttt ggggggtaat cagcattatt cttcagaagg ggacctgttt tottcaaggg |
| 3361 | aagaaacact cttattccca aactacagaa taatgtgtta aacatgctaa atagttctat |
| 3421 | caggaaaaca aatcactgta tttatctccg caggctattt gttcagagag gccttttgtt |
| 3481 | taaatataaa tgtttaaata taaatgtttg tctggattgg ctataacatg tctttcagca |
| 3541 | ttaggctttt aagaaacaca gggttttgta ttctttacta aagatatcag agctcttaat |
| 3601 | gttgcttaga tgagggtgac tgtcaagtac aagcaagact gggaccttag aaatcattgt |
| 3661 | agaaacacag ttttgaaaga aaaataccat gtctctaagc caactttaat tgcttaaaag |
| 3721 | acatttttat ttagttgaaa aatctagttt tttttgtaaa ctgtatcaaa tctgtatatg |
| 3781 | ttgtaataaa acttatgcta gtttattgga agtgttcaag aaataaaaat caacttgtgt |
| 3841 | actgataaaa tactctagcc tgggccagag aagataatgt tctttaatgt tgtccaggaa |
| 3901 | accctggctt gcttgccgag cctaatgaaa gggaaagtca gctttcagag ccagtgaagg |
| 3961 | agccacgtga atggccctag aactgtgcct agttcctgtg gccaggaggt tggtgactga |
| 4021 | aacattcaca cagggctctt tgatggaccc acgaacgctc ttagctttct cagggggtca |
| 4081 | gcagagttat tgaatcttaa ttttttttaa tgtacaagtt ttgtataaat aataaagaac |
| 4141 | tccttatttt gtattacatc taatgcttca agtgttgctc ttggaaagct gatgatgtct |
| 4201 | cttgtagaag atggactctg aaaaacattc caggaaacca tggcagcatg gagagcctct |
| 4261 | tagtgattgt gtctgcattg ttattgtgga agatttacct tttctgttgt acgtaaagct |
| 4321 | taaattgctt ttgttgtgac tttttagcca gtgacttttt ctgagetttt catggaagtg |
| 4381 | gcagtgaaaa atatgttgag tgttcatttt agtgactgta attaatatct tgctggatta |
| 4441 | atgttttgta caattactaa attgtataca ttttgttata gaatactttt ttctagtttc |
| 4501 | agtaaataat gaaaaggaag ttaataccaa |

In Table 1, each code (letter, e.g., A, G, C, and U) represents a single ribonucleotide in the dsRNA. In some embodiments, the sequence list may be inclusive of any possible, additional modifications in a nucleobase, a ribose sugar ring, and/or a phosphate group (i.e., internucleoside linkage). In some embodiments, the last nucleotide from the 5′ end (or the first nucleotide from 3′ end) in each strand (sense strand and antisense strand) may have not include a phosphate group as being hydrolyzed or processed, e.g., during the synthesis of the oligonucleotides, but may contain 3′-terminal —OH group. In some embodiments, a phosphate group in the last nucleotide from the 5′ end (or the first nucleotide from 3′ end) in the sense strand may be added as a functional group for conjugation with a ligand.

In some embodiments, the dsRNA includes a sense strand having 10 contiguous nucleotides differing by no more than 3 nucleotides from the nucleotide sequence selected from SEQ ID NOs: 1 to 405 and 1434 to 1440. In some embodiments, the dsRNA includes an antisense strand having 10 contiguous nucleotides differing by no more than 3 nucleotides from the nucleotide sequence of SEQ ID NOs: 406 to 810 and 1441 to 1447. In some embodiments, the dsRNA includes a sense strand having 11 contiguous nucleotides differing by no more than 3 nucleotides from the nucleotide sequence selected from SEQ ID NOs: 1 to 405 and 1434 to 1440. In some embodiments, the dsRNA includes an antisense strand having 11 contiguous nucleotides differing by no more than 3 nucleotides from the nucleotide sequence selected from SEQ ID NOs: 406 to 810 and 1441 to 1447. In some embodiments, the dsRNA includes a sense strand having 12 contiguous nucleotides differing by no more than 3 nucleotides from the nucleotide sequence selected from SEQ ID NOs: 1 to 405 and 1434 to 1440. In some embodiments, the dsRNA includes an antisense strand having 12 contiguous nucleotides differing by no more than 3 nucleotides from the nucleotide sequence selected from SEQ ID NOs: 406 to 810 and 1441 to 1447. In some embodiments, the dsRNA includes a sense strand having 13 contiguous nucleotides differing by no more than 3 nucleotides from the nucleotide sequence selected from SEQ ID NOs: 1 to 405 and 1434 to 1440. In some embodiments, the dsRNA includes an antisense strand having 13 contiguous nucleotides differing by no more than 3 nucleotides from the nucleotide sequence selected from SEQ ID NOs: 406 to 810 and 1441 to 1447. In some embodiments, the dsRNA includes a sense strand having 14 contiguous nucleotides differing by no more than 3 nucleotides from the nucleotide sequence selected from SEQ ID NOs: 1 to 405 and 1434 to 1440. In some embodiments, the dsRNA includes an antisense strand having 14 contiguous nucleotides differing by no more than 3 nucleotides from the nucleotide sequence selected from SEQ ID NOs: 406 to 810 and 1441 to 1447. In some embodiments, the dsRNA includes a sense strand having 15 contiguous nucleotides differing by no more than 3 nucleotides from the nucleotide sequence selected from SEQ ID NOs: 1 to 405 and 1434 to 1440. In some embodiments, the dsRNA includes an antisense strand having 15 contiguous nucleotides differing by no more than 3 nucleotides from the nucleotide sequence selected from SEQ ID NOs: 406 to 810 and 1441 to 1447. In some embodiments, the dsRNA includes a sense strand having 16 contiguous nucleotides differing by no more than 3 nucleotides from the nucleotide sequence selected from SEQ ID NOs: 1 to 405 and 1434 to 1440. In some embodiments, the dsRNA includes an antisense strand having 16 contiguous nucleotides differing by no more than 3 nucleotides from the nucleotide sequence selected from SEQ ID NOs: 406 to 810 and 1441 to 1447. In some embodiments, the dsRNA includes a sense strand having 17 contiguous nucleotides differing by no more than 3 nucleotides from the nucleotide sequence selected from SEQ ID NOs: 1 to 405 and 1434 to 1440. In some embodiments, the dsRNA includes an antisense strand having 17 contiguous nucleotides differing by no more than 3 nucleotides from the nucleotide sequence selected from SEQ ID NOs: 406 to 810 and 1441 to 1447. In some embodiments, the dsRNA includes a sense strand having 18 contiguous nucleotides differing by no more than 3 nucleotides from the nucleotide sequence selected from SEQ ID NOs: 1 to 405 and 1434 to 1440. In some embodiments, the dsRNA includes an antisense strand having 18 contiguous nucleotides differing by no more than 3 nucleotides from the nucleotide sequence selected from SEQ ID NOs: 406 to 810 and 1441 to 1447. In some embodiments, the dsRNA includes a sense strand having 19 contiguous nucleotides differing by no more than 3 nucleotides from the nucleotide sequence selected from SEQ ID NOs: 1 to 405 and 1434 to 1440. In some embodiments, the dsRNA includes an antisense strand having 19 contiguous nucleotides differing by no more than 3 nucleotides from the nucleotide sequence selected from SEQ ID NOs: 406 to 810 and 1441 to 1447. In some embodiments, the dsRNA includes a sense strand having 20 contiguous nucleotides differing by no more than 3 nucleotides from the nucleotide sequence selected from SEQ ID NOs: 1 to 405 and 1434 to 1440. In some embodiments, the dsRNA includes an antisense strand having 20 contiguous nucleotides differing by no more than 3 nucleotides from the nucleotide sequence selected from SEQ ID NOs: 406 to 810 and 1441 to 1447. In some embodiments, the dsRNA includes a sense strand having 21 contiguous nucleotides differing by no more than 3 nucleotides from the nucleotide sequence selected from SEQ ID NOs: 1 to 405 and 1434 to 1440. In some embodiments, the dsRNA includes an antisense strand having 21 contiguous nucleotides differing by no more than 3 nucleotides from the nucleotide sequence selected from SEQ ID NOs: 406 to 810 and 1441 to 1447.

In some embodiments, the dsRNA includes a sense strand having 10 contiguous nucleotides differing by no more than 2 nucleotides from the nucleotide sequence selected from SEQ ID NOs: 1 to 405 and 1434 to 1440. In some embodiments, the dsRNA includes an antisense strand having 10 contiguous nucleotides differing by no more than 2 nucleotides from the nucleotide sequence selected from SEQ ID NOs: 406 to 810 and 1441 to 1447. In some embodiments, the dsRNA includes a sense strand having 11 contiguous nucleotides differing by no more than 2 nucleotides from the nucleotide sequence selected from SEQ ID NOs: 1 to 405 and 1434 to 1440. In some embodiments, the dsRNA includes an antisense strand having 11 contiguous nucleotides differing by no more than 2 nucleotides from the nucleotide sequence selected from SEQ ID NOs: 406 to 810 and 1441 to 1447. In some embodiments, the dsRNA includes a sense strand having 12 contiguous nucleotides differing by no more than 2 nucleotides from the nucleotide sequence selected from SEQ ID NOs: 1 to 405 and 1434 to 1440. In some embodiments, the dsRNA includes an antisense strand having 12 contiguous nucleotides differing by no more than 2 nucleotides from the nucleotide sequence selected from SEQ ID NOs: 406 to 810 and 1441 to 1447. In some embodiments, the dsRNA includes a sense strand having 13 contiguous nucleotides differing by no more than 2 nucleotides from the nucleotide sequence selected from SEQ ID NOs: 1 to 405 and 1434 to 1440. In some embodiments, the dsRNA includes an antisense strand having 13 contiguous nucleotides differing by no more than 2 nucleotides from the nucleotide sequence selected from SEQ ID NOs: 406 to 810 and 1441 to 1447. In some embodiments, the dsRNA includes a sense strand having 14 contiguous nucleotides differing by no more than 2 nucleotides from the nucleotide sequence selected from SEQ ID NOs: 1 to 405 and 1434 to 1440. In some embodiments, the dsRNA includes an antisense strand having 14 contiguous nucleotides differing by no more than 2 nucleotides from the nucleotide sequence selected from SEQ ID NOs: 406 to 810 and 1441 to 1447. In some embodiments, the dsRNA includes a sense strand having 15 contiguous nucleotides differing by no more than 2 nucleotides from the nucleotide sequence selected from SEQ ID NOs: 1 to 405 and 1434 to 1440. In some embodiments, the dsRNA includes an antisense strand having 15 contiguous nucleotides differing by no more than 2 nucleotides from the nucleotide sequence selected from SEQ ID NOs: 406 to 810 and 1441 to 1447. In some embodiments, the dsRNA includes a sense strand having 16 contiguous nucleotides differing by no more than 2 nucleotides from the nucleotide sequence selected from SEQ ID NOs: 1 to 405 and 1434 to 1440. In some embodiments, the dsRNA includes an antisense strand having 16 contiguous nucleotides differing by no more than 2 nucleotides from the nucleotide sequence selected from SEQ ID NOs: 406 to 810 and 1441 to 1447. In some embodiments, the dsRNA includes a sense strand having 17 contiguous nucleotides differing by no more than 2 nucleotides from the nucleotide sequence selected from SEQ ID NOs: 1 to 405 and 1434 to 1440. In some embodiments, the dsRNA includes an antisense strand having 17 contiguous nucleotides differing by no more than 2 nucleotides from the nucleotide sequence selected from SEQ ID NOs: 406 to 810 and 1441 to 1447. In some embodiments, the dsRNA includes a sense strand having 18 contiguous nucleotides differing by no more than 2 nucleotides from the nucleotide sequence selected from SEQ ID NOs: 1 to 405 and 1434 to 1440. In some embodiments, the dsRNA includes an antisense strand having 18 contiguous nucleotides differing by no more than 2 nucleotides from the nucleotide sequence selected from SEQ ID NOs: 406 to 810 and 1441 to 1447. In some embodiments, the dsRNA includes a sense strand having 19 contiguous nucleotides differing by no more than 2 nucleotides from the nucleotide sequence selected from SEQ ID NOs: 1 to 405 and 1434 to 1440. In some embodiments, the dsRNA includes an antisense strand having 19 contiguous nucleotides differing by no more than 2 nucleotides from the nucleotide sequence selected from SEQ ID NOs: 406 to 810 and 1441 to 1447. In some embodiments, the dsRNA includes a sense strand having 20 contiguous nucleotides differing by no more than 2 nucleotides from the nucleotide sequence selected from SEQ ID NOs: 1 to 405 and 1434 to 1440. In some embodiments, the dsRNA includes an antisense strand having 20 contiguous nucleotides differing by no more than 2 nucleotides from the nucleotide sequence selected from SEQ ID NOs: 406 to 810 and 1441 to 1447. In some embodiments, the dsRNA includes a sense strand having 21 contiguous nucleotides differing by no more than 2 nucleotides from the nucleotide sequence selected from SEQ ID NOs: 1 to 405 and 1434 to 1440. In some embodiments, the dsRNA includes an antisense strand having 21 contiguous nucleotides differing by no more than 2 nucleotides from the nucleotide sequence selected from SEQ ID NOs: 406 to 810 and 1441 to 1447.

In some embodiments, the dsRNA includes a sense strand having 10 contiguous nucleotides differing by no more than 1 nucleotide from the nucleotide sequence selected from SEQ ID NOs: 1 to 405 and 1434 to 1440. In some embodiments, the dsRNA includes an antisense strand having 10 contiguous nucleotides differing by no more than 1 nucleotide from the nucleotide sequence selected from SEQ ID NOs: 406 to 810 and 1441 to 1447. In some embodiments, the dsRNA includes a sense strand having 11 contiguous nucleotides differing by no more than 1 nucleotide from the nucleotide sequence selected from SEQ ID NOs: 1 to 405 and 1434 to 1440. In some embodiments, the dsRNA includes an antisense strand having 11 contiguous nucleotides differing by no more than 1 nucleotide from the nucleotide sequence selected from SEQ ID NOs: 406 to 810 and 1441 to 1447. In some embodiments, the dsRNA includes a sense strand having 12 contiguous nucleotides differing by no more than 1 nucleotide from the nucleotide sequence selected from SEQ ID NOs: 1 to 405 and 1434 to 1440. In some embodiments, the dsRNA includes an antisense strand having 12 contiguous nucleotides differing by no more than 1 nucleotide from the nucleotide sequence selected from SEQ ID NOs: 406 to 810 and 1441 to 1447. In some embodiments, the dsRNA includes a sense strand having 13 contiguous nucleotides differing by no more than 1 nucleotide from the nucleotide sequence selected from SEQ ID NOs: 1 to 405 and 1434 to 1440. In some embodiments, the dsRNA includes an antisense strand having 13 contiguous nucleotides differing by no more than 1 nucleotide from the nucleotide sequence selected from SEQ ID NOs: 406 to 810 and 1441 to 1447. In some embodiments, the dsRNA includes a sense strand having 14 contiguous nucleotides differing by no more than 1 nucleotide from the nucleotide sequence selected from SEQ ID NOs: 1 to 405 and 1434 to 1440. In some embodiments, the dsRNA includes an antisense strand having 14 contiguous nucleotides differing by no more than 1 nucleotide from the nucleotide sequence selected from SEQ ID NOs: 406 to 810 and 1441 to 1447. In some embodiments, the dsRNA includes a sense strand having 15 contiguous nucleotides differing by no more than 1 nucleotide from the nucleotide sequence selected from SEQ ID NOs: 1 to 405 and 1434 to 1440. In some embodiments, the dsRNA includes an antisense strand having 15 contiguous nucleotides differing by no more than 1 nucleotide from the nucleotide sequence selected from SEQ ID NOs: 406 to 810 and 1441 to 1447. In some embodiments, the dsRNA includes a sense strand having 16 contiguous nucleotides differing by no more than 1 nucleotide from the nucleotide sequence selected from SEQ ID NOs: 1 to 405 and 1434 to 1440. In some embodiments, the dsRNA includes an antisense strand having 16 contiguous nucleotides differing by no more than 1 nucleotide from the nucleotide sequence selected from SEQ ID NOs: 406 to 810 and 1441 to 1447. In some embodiments, the dsRNA includes a sense strand having 17 contiguous nucleotides differing by no more than 1 nucleotide from the nucleotide sequence selected from SEQ ID NOs: 1 to 405 and 1434 to 1440. In some embodiments, the dsRNA includes an antisense strand having 17 contiguous nucleotides differing by no more than 1 nucleotide from the nucleotide sequence selected from SEQ ID NOs: 406 to 810 and 1441 to 1447. In some embodiments, the dsRNA includes a sense strand having 18 contiguous nucleotides differing by no more than 1 nucleotide from the nucleotide sequence selected from SEQ ID NOs: 1 to 405 and 1434 to 1440. In some embodiments, the dsRNA includes an antisense strand having 18 contiguous nucleotides differing by no more than 1 nucleotide from the nucleotide sequence selected from SEQ ID NOs: 406 to 810 and 1441 to 1447. In some embodiments, the dsRNA includes a sense strand having 19 contiguous nucleotides differing by no more than 1 nucleotide from the nucleotide sequence selected from SEQ ID NOs: 1 to 405 and 1434 to 1440. In some embodiments, the dsRNA includes an antisense strand having 19 contiguous nucleotides differing by no more than 1 nucleotide from the nucleotide sequence selected from SEQ ID NOs: 406 to 810 and 1441 to 1447. In some embodiments, the dsRNA includes a sense strand having 20 contiguous nucleotides differing by no more than 1 nucleotide from the nucleotide sequence selected from SEQ ID NOs: 1 to 405 and 1434 to 1440. In some embodiments, the dsRNA includes an antisense strand having 20 contiguous nucleotides differing by no more than 1 nucleotide from the nucleotide sequence selected from SEQ ID NOs: 406 to 810 and 1441 to 1447. In some embodiments, the dsRNA includes a sense strand having 21 contiguous nucleotides differing by no more than 1 nucleotide from the nucleotide sequence selected from SEQ ID NOs: 1 to 405 and 1434 to 1440. In some embodiments, the dsRNA includes an antisense strand having 21 contiguous nucleotides differing by no more than 1 nucleotide from the nucleotide sequence selected from SEQ ID NOs: 406 to 810 and 1441 to 1447. In some embodiments, the dsRNA includes an antisense strand having 22 contiguous nucleotides differing by no more than 1 nucleotide from the nucleotide sequence selected from SEQ ID NOs: 406 to 810 and 1441 to 1447. In some embodiments, the dsRNA includes an antisense strand having 23 contiguous nucleotides differing by no more than 1 nucleotide from the nucleotide sequence selected from SEQ ID NOs: 406 to 810 and 1441 to 1447.

In some embodiments, the dsRNA includes (i) a sense strand having 15 contiguous nucleotides differing by no more than one, two or three nucleotides from the nucleotide sequence selected from SEQ ID NOs: 1 to 405 and 1434 to 1440 and (ii) an antisense strand forming a duplex with the sense strand of (i) and having 15 contiguous nucleotides differing by no more than one, two or three nucleotides from the nucleotide sequence selected from SEQ ID NOs: 406 to 810 and 1441 to 1447. In some embodiments, the dsRNA includes (i) a sense strand having 16 contiguous nucleotides differing by no more than one, two or three from the nucleotide sequence selected from SEQ ID NOs: 1 to 405 and 1434 to 1440 and (ii) an antisense strand forming a duplex with the sense strand of (i) and having 16 contiguous nucleotides differing by no more than one, two or three nucleotides from the nucleotide sequence selected from SEQ ID NOs: 406 to 810 and 1441 to 1447. In some embodiments, the dsRNA includes (i) a sense strand having 17 contiguous nucleotides differing by no more than one, two or three nucleotides from the nucleotide sequence selected from SEQ ID NOs: 1 to 405 and 1434 to 1440 and (ii) an antisense strand forming a duplex with the sense strand of (i) and having 17 contiguous nucleotides differing by no more than one, two or three nucleotides from the nucleotide sequence selected from SEQ ID NOs: 406 to 810 and 1441 to 1447. In some embodiments, the dsRNA includes (i) a sense strand having 18 contiguous nucleotides differing by no more than one, two or three nucleotides from the nucleotide sequence selected from SEQ ID NOs: 1 to 405 and 1434 to 1440 and (ii) an antisense strand forming a duplex with the sense strand of (i) and having 18 contiguous nucleotides differing by no more than one, two or three nucleotides from the nucleotide sequence selected from SEQ ID NOs: 406 to 810 and 1441 to 1447. In some embodiments, the dsRNA includes (i) a sense strand having 19 contiguous nucleotides differing by no more than one, two or three nucleotides from the nucleotide sequence selected from SEQ ID NOs: 1 to 405 and 1434 to 1440 and (ii) an antisense strand forming a duplex with the sense strand of (i) and having 19 contiguous nucleotides differing by no more than one, two or three nucleotides from the nucleotide sequence selected from SEQ ID NOs: 406 to 810 and 1441 to 1447. In some embodiments, the dsRNA includes (i) a sense strand having 20 contiguous nucleotides differing by no more than one, two or three nucleotides from the nucleotide sequence selected from SEQ ID NOs: 1 to 405 and 1434 to 1440 and (ii) an antisense strand forming a duplex with the sense strand of (i) and having 20 contiguous nucleotides differing by no more than one, two or three nucleotides from the nucleotide sequence selected from SEQ ID NOs: 406 to 810 and 1441 to 1447. In some embodiments, the dsRNA includes (i) a sense strand having 21 contiguous nucleotides differing by no more than one, two or three nucleotides from the nucleotide sequence selected from SEQ ID NOs: 1 to 405 and 1434 to 1440 and (ii) an antisense strand forming a duplex with the sense strand of (i) and having 21 contiguous nucleotides differing by no more than one, two or three nucleotides from the nucleotide sequence selected from SEQ ID NOs: 406 to 810 and 1441 to 1447.

In certain aspects, when a sense strand or an antisense strand of a dsRNA in above paragraphs is differing by a certain number of nucleotides (e.g., one, two or three nucleotides) from a specific sequence (e.g., SEQ ID NOs: 1 to 810 and 1434 to 1447), it is meant by that the sense strand or the antisense strand of the dsRNA includes one, two or three nucleotides having different nucleobases compared to the nucleobases of the nucleotides at the corresponding positions of the specific sequence (e.g., SEQ ID NOs: 1 to 810 and 1434 to 1447).

### Modification Pattern

In an aspect, the disclosure provides a set of modification patterns determined or arranged by modified nucleotides in dsRNAs described herein. Aside from or in addition to the nucleobase sequences, various arrangements of modified nucleotides and the modification patterns thereof can be introduced, for example, to increase stability in a biological or physiological surrounding, to facilitate or promote cleavage by the RNA-induced silencing complex, and/or to mitigate or reduce off-targeting risk (e.g., to HMGCR off-targeting risk).

In an aspect, the disclosure provides a dsRNA that is partially (e.g., greater than about 1%, 5%, 10%, 15%, 20%, 25%, 30%, 35%, 40%, or 45% of the total nucleotides), substantially (e.g., greater than about 50%, 55%, 60%, 65%, 70%, 75%, 80%, 85%, 90%, or 95% of the total nucleotides), or entirely made of modified nucleotides, which can provide improved resistance to chemical and/or nuclease digestion and increased in vivo stability thereby imposing a longer in vivo half-life. Further, increasing the in vivo half-life of the dsRNA results in enhanced bioavailability and enhanced effectiveness in inhibiting expression or activity of a target gene (e.g., human HMGCR). For example, the stability of dsRNA in blood or serum may be determined, e.g., by its susceptibility to degradation by the cellular enzymes, which may be dependent on the characteristics (e.g., sequences, modification, modification pattern, or other chemical moieties) of each strand (i.e., sense strand or antisense strand) of the dsRNA. Thus, in certain aspect, the efficiency of dsRNA as a therapeutic agent may be improved by increasing the in vivo stability (e.g., in blood or serum) of the dsRNA while maintaining the ability of the dsRNA to mediate RNA interference in vivo.

### Modified Nucleotides

The modified nucleotides as used herein contain one or more modifications, for example, the modified nucleotides contain at least one chemical modification or replacement in an internucleoside linkage (“linkage”), a nucleobase, and/or a sugar moiety of the nucleotide. Non-limiting examples include a 2′-modification on a ribose sugar ring (e.g., 2′-deoxy, 2′-O-alkyl, 2′-halo, 2′-O-alkoxyalkyl, 2′-O-amino alkyl, etc.), 3′-modification (e.g., substitution) in backbone phosphate group, or 4′-modification on a ribose sugar ring (e.g., 4′-thio RNA). Also, other non-limiting examples of modifications may include one or more modifications selected from a deoxy modification, a 2′-O-alkyl modification, a 2′-halo modification, a 2′-5′-linkage modification, a conformationally restricting modification, an abasic modification, a 2′amino-modification, a 2′-O-allyl modification, 2′-C-alkyl modification, a 2′-O-alkoxyalkyl modification, a morpholino modification, a modification containing a phosphoramidate group, a modification containing a non-natural nucleobase, a modification in a tetrahydropyran, a modification in a threose (TNA), a modification containing a 1,5-anhydrohexitol, a modification containing a cyclohexyl, a modification containing a cyclohexenyl a modification containing a phosphorothioate group, a modification containing a methylphosphonate group, a modification containing an alkylphosphate, a modification containing a phosphonate, a modification containing an alkylphosphonate, a modification to form a thermally destabilizing nucleotide, a modification containing glycol (GNA), and a 2-O-(N-methylacetamide) modification. For example, a modified nucleotide may include a single modification, or two or more modifications at the positions at which the chemical modification groups do not hinder or intervene each other.

In some embodiments, each of the modified nucleotides is independently selected from LNA, GNA, TNA, 2′-O-alkoxyalkyl modified nucleotide, 2′-O-alkyl modified nucleotide, 2-O-allyl modified nucleotide, 2′C-allyl modified nucleotide, 2′-halo modified nucleotide, and 2˜deoxy modified nucleotide (DNA). The term alkyl, alkoxyl, allyl, amino, and halo can be interpreted as described above. In some embodiments, the modified nucleotides include at least one LNAs. In some embodiments, the modified nucleotides include at least one GNAs. In some embodiments, the modified nucleotides include at least one TNAs. In some embodiments, the modified nucleotides include at least one 2′-O-alkoxyalkyl modified nucleotides. In some embodiments, the modified nucleotides include at least one 2′-O-alkyl modified nucleotides. In some embodiments, the modified nucleotides include at least one 2-O-allyl modified nucleotides. In some embodiments, the modified nucleotides include at least one 2′-C-allyl modified nucleotides. In some embodiments, the modified nucleotides include at least one 2′-halo (e.g., —F) modified nucleotides. In some embodiments, the modified nucleotides include at least one 2′-deoxy modified nucleotides (DNA).

In some embodiments, each of the modified nucleotides contain independently selected from LNA modification, GNA modification, TNA modification, 2′-O-alkoxyalkyl modification, 2′-O-alkyl modification, 2′-O-allyl modification, 2′-C-allyl modification, 2′-halo modification, and 2′-deoxy modification (DNA). The term alkyl, alkoxyl, allyl, amino, and halo can be interpreted as described above. In some embodiments, the modified nucleotides include at least one LNAs. In some embodiments, the modified nucleotides include at least one GNAs. In some embodiments, the modified nucleotides include at least one TNAs. In some embodiments, the modified nucleotides include at least one 2′-O-alkoxyalkyl modifications. In some embodiments, the modified nucleotides include at least one 2′-O-alkyl modifications. In some embodiments, the modified nucleotides include at least one 2′-O-allyl modifications. In some embodiments, the modified nucleotides include at least one 2′-C-allyl modifications. In some embodiments, the modified nucleotides include at least one 2′-halo (e.g., —F) modifications. In some embodiments, the modified nucleotides include at least one 2′-deoxy modifications (DNA).

In some embodiments, the modified nucleotide may be a bicyclic (or bridged) nucleic acid (“BNA”) having a covalent linkage between the 2′ and 4′ carbons on a ribose sugar. In some embodiments, the modified nucleotide is a locked RNA (“LNA”) having covalent linkage of a bicyclic sugar modification is a 4′-CH₂-O-2′ linkage (methylene oxy), also known as LNA having a structure of e.g.,

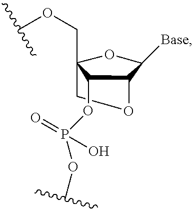

or a pharmaceutically acceptable salt thereof.

In some embodiments, a ribose ring may be replaced with a glycol motif linked to phosphate and the GNA nucleotide has a structure of

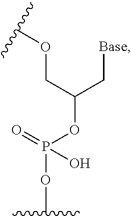

or a pharmaceutically acceptable salt thereof.

In some embodiments, a ribose pentofuranosyl ring may be replaced with a threofuranosyl ring linked to the phosphate and a threofuranosyl nucleotide (TNA) may include a moiety of

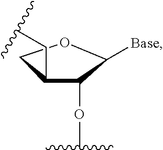

or a pharmaceutically acceptable salt thereof. In some embodiments, the TNA may have a structure of

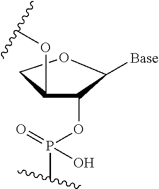

or a pharmaceutically acceptable salt thereof. In some embodiments, the phosphodiester linkage in the TNA may be modified, e.g., with phosphorothioate group and modified TNA may include a structure of

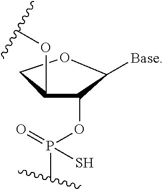

or a pharmaceutically acceptable salt thereof. The TNA may further include one or more substituents at 1′, 3′ and/or 4′ positions and such modified TNA may be encompassed by the definition of TNA herein.

In some embodiments, a ribose ring may not include a base and an abasic nucleotide has a structure of

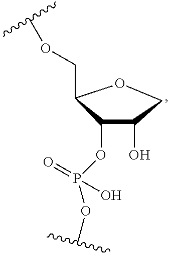

or a pharmaceutically acceptable salt thereof.

In certain aspects, the modified nucleotide may include a heterocyclic group (e.g., 5 to 6 membered heterocycloalkyl ring) in place of a ribose ring. In some embodiments, the ribose ring may be replaced with a morpholinyl ring, e.g., to form an morpholino oligonucleotide. In some embodiments, the ribose ring may be replaced with an arabinose ring.

In certain aspects, the modified nucleotides contain one or more modification groups at 2′ position on the ribose ring by replacing 2′-OH. In some embodiments, the modification group may be hydrogen (i.e. deoxy), halogen (e.g., —F), substituted or unsubstituted alkyl (e.g., C₁-C₁₂ alkyl), or substituted or unsubstituted heteroalkyl (e.g., —O—(C₁-C₁₂ alkyl), —N—(C₁-C₁₂ alkyl), —C(O)NH—(C₁-C₁₂ alkyl), —NHC(O)—(C₁-C₁₂ alkyl), or —C(O)—(C₁-C₁₂ alkyl)). In some embodiments, the modification group may be hydrogen, —F, —O-alkyl (e.g., C₁-C₄ alkyl), or —O— alkoxyalkyl (e.g., —O—(C₁-C₄ alkylene)-(C₁-C₄ alkoxyl)). Any of the alkyl, heteroalkyl, alkylene in the disclosure are optionally substituted with one or more of hydroxyl (—OH), C₁-C₃ alkyl (e.g., methyl, or ethyl), amine (e.g., monoamine or diamine), alkoxyl (e.g., —O—CH₃ (OMe) or —O—CH₂CH₃ (OEt)), halogen (e.g., —F) or the like.

In certain aspects, the modified nucleotides may include one or more of 2′-deoxy modification, 2′-O-alkyl modification, 2′-O-substituted alkyl modification, 2′-O-alkoxyalkyl modification, and 2′-O-aminoalkyl modification. In some embodiments, the modified nucleotides may include one or more of 2′-deoxy modification, 2′-O-alkyl modification, 2′-O—substituted alkyl modification, 2′-O-alkoxyalkyl modification, and 2′-O-aminoalkyl modification. In some embodiments, the modified nucleotides include at least one GNAs. In some embodiments, the modified nucleotides include at least one 2′-O-alkoxyalkyl modifications. In some embodiments, the modified nucleotides include at least one 2′-O-alkyl modifications. In some embodiments, the modified nucleotides include at least one 2′-O-allyl modifications. In some embodiments, the modified nucleotides include at least one 2′-C-allyl modifications. In some embodiments, the modified nucleotides include at least one 2′-halo (e.g., —F) modifications. In some embodiments, the modified nucleotides include at least one 2′-deoxy modifications (DNA). In some embodiments, the modified nucleotides do not include 2′-deoxy modifications (DNA).

In certain aspects, the modified nucleotides may include one or more of 2′-deoxy nucleotide (DNA), 2′-O-methyl (2′-OMe) modification, 2′-flouro (2′-F) modification, 2′-O—methoxyethyl (2′-O-MOE or “2′-MOE”) modification, 2′-O-aminopropyl (2′-O-AP) modification, 2′-O-dimethylaminoethyl (2′-O-DMAOE) modification, 2′-O—dimethylaminopropyl (2′-O-DMAP) modification, 2′-O-dimethylaminoethyloxyethyl (2′-O—DMAEOE) modification, and 2′-O-N-methylacetamido (2′-O-NMA) modification. In some embodiments, the modified nucleotides may include at least one 2′-deoxy modification (DNA). In some embodiments, the modified nucleotides may include at least one 2′-O-methyl (2′-OMe) modification. In some embodiments, the modified nucleotides may include at least one 2′-flouro (2′-F) modification. In some embodiments, the modified nucleotides may include at least one 2′-0-methoxyethyl (2′-O-MOE or “2′-MOE”) modification. In some embodiments, the modified nucleotides may include at least one 2′-O-aminopropyl (2′-O-AP) modification. In some embodiments, the modified nucleotides may include at least one 2′-O-dimethylaminoethyl (2′-O—DMAOE) modification. In some embodiments, the modified nucleotides may include at least one 2′-O-dimethylaminopropyl (2′-O-DMAP) modification. In some embodiments, the modified nucleotides may include at least one 2′-O-dimethylaminoethyloxyethyl (2′-O-DMAEOE) modification. In some embodiments, the modified nucleotides may include at least one 2′-O-N-methylacetamido (2′-O-NMA) modification.

In some embodiments, each modified nucleotide containing a modification on a 2′ sugar ring may optionally contain a phosphorothioate group at 5′ or 3′ linkage. In some embodiments, each modified nucleotide containing a modification on a 2′ sugar ring may optionally contain a modification such as an abasic modification or methylated nucleobase modification at nucleobase.

In certain aspects, the dsRNA is partially (e.g., greater than about 1%, 5%, 10%, 15%, 20%, 25%, 30%, 35%, 40%, or 45% of the total nucleotides), substantially (e.g., greater than about 50%, 55%, 60%, 65%, 70%, 75%, 80%, 85%, 90%, or 95% of the total nucleotides), or entirely made of modified nucleotides containing the modification on 2′ sugar ring. In some embodiments, the dsRNA is partially (e.g., greater than about 1%, 5%, 10%, 15%, 20%, 25%, 30%, 35%, 40%, or 45% of the total nucleotides) made of modified nucleotides containing the modification on 2′ sugar ring. In some embodiments, the dsRNA is substantially (e.g., greater than about 50%, 55%, 60%, 65%, 70%, 75%, 80%, 85%, 90%, or 95% of the total nucleotides) made of modified nucleotides containing the modification on 2′ sugar ring. In some embodiments, the dsRNA includes greater than about 80% of modified nucleotides containing the modification on 2′ sugar ring based on the total nucleotides. In some embodiments, the dsRNA includes greater than about 85% of modified nucleotides containing the modification on 2′ sugar ring based on the total nucleotides. In some embodiments, the dsRNA includes greater than about 90% of modified nucleotides containing the modification on 2′ sugar ring based on the total nucleotides. In some embodiments, the dsRNA includes greater than about 95% of modified nucleotides containing the modification on 2′ sugar ring based on the total nucleotides. In some embodiments, the dsRNA is entirely made of modified nucleotides containing the modification on 2′ sugar ring.

In certain aspects, the modified nucleotide may include a modification in a phosphate group or, in other words, an internucleoside linkage modification (e.g., phosphorothioate, phosphorodithioate, methylphosphonate, methylene phosphonate, or vinylphosphonate (VP) linkage). In some embodiments, the linkage modification may include phosphorothioate (PS) having a structure of

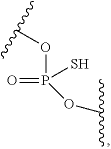 [SMILES: *OP(=O)(S)O*]

which may be an Rp isomer or an Sp isomer. In some embodiments, the linkage modification may include phosphorothioate (PS) having a structure of

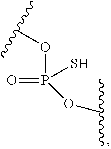 [SMILES: *OP(=O)(S)O*]

which may be a stereopure Rp isomer. In some embodiments, the linkage modification may include phosphorothioate (PS) having a structure of

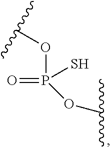 [SMILES: *OP(=O)(S)O*]

which may be a stereopure Sp isomer.

For example, the modified nucleotide including 3′-PS modification can be represented as

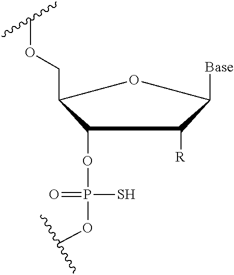

wherein R represents H, OH or a substituent (e.g., —F, —CH₃, —OMe, or MOE). In some embodiments, the 3′-PS group may be a stereopure Sp isomer. In some embodiments, the 3′-PS group may be a stereopure Rp isomer.

In certain aspects, the dsRNAi agent may be entirely made of modified nucleotides having one or more internucleoside linkage modification and/or modifications in the sugar moieties of the nucleotides. Example dsRNA (siRNA) with modified nucleotides, including sense strands and antisense strands targeting the above indicated HMGCR mRNA, are shown in Table 2.

TABLE 2

| | position | | SEQ | | SEQ |
|---|---|---|---|---|---|
| siRNA | in | | ID | | ID |
| No. | mRNA | Sense Strand | NO: | Antisense strand | NO: |
| 406 | 125 | A004p001U004p001G004pU004 | 812 | U004p001C007p001G004pA004p | 1053 |
| | | pU004pG004pU007pC004pA007 | | A004pA007pA004pA004pG004pU | |
| | | pA007pG007pA004pC004pU004 | | 004pC004pU004pU004pG007pA0 | |
| | | pU004pU004pU004pU004pC004 | | 04pC007pA004pA004pC004pA00 | |
| | | pG004pA004 | | 4pU004p001U004p001G004 | |
| 407 | 126 | U004p001G004p001U004pU004 | 813 | U004p001U007p001C004pG004p | 1054 |
| | | pG004pU004pC007pA004pA007 | | A004pA007pA004pA004pA004pG | |
| | | pG007pA007pC004pU004pU004 | | 004pU004pC004pU004pU007pG0 | |
| | | pU004pU004pU004pC004pG004 | | 04pA007pC004pA004pA004pC00 | |
| | | pA004pA004 | | 4pA004p001U004p001U004 | |
| 408 | 127 | G004p001U004p001U004pG004 | 814 | A004p001U007p001U004pC004p | 1055 |
| | | pU004pC004pA007pA004pG007 | | G004pA007pA004pA004pA004pA | |
| | | pA007pC007pU004pU004pU004 | | 004pG004pU004pC004pU007pU0 | |
| | | pU004pU004pC004pG004pA004 | | 04pG007pA004pC004pA004pA00 | |
| | | pA004pU004 | | 4pC004p001A004p001U004 | |
| 409 | 130 | G004p001U004p001C004pA004 | 815 | U004p001G007p001C004pA004p | 1056 |
| | | pA004pG004pA007pC004pU007 | | U004pU007pC004pG004pA004pA | |
| | | pU007pU007pU004pU004pC004 | | 004pA004pA004pA004pG007pU0 | |
| | | pG004pA004pA004pU004pG004 | | 04pC007pU004pU004pG004pA00 | |
| | | pC004pA004 | | 4pC004p001A004p001A004 | |
| 410 | 131 | U004p001C004p001A004pA004 | 816 | A004p001U007p001G004pC004p | 1057 |
| | | pG004pA004pC007pU004pU007 | | A004pU007pU004pC004pG004pA | |
| | | pU007pU007pU004pC004pG004 | | 004pA004pA004pA004pA007pG0 | |
| | | pA004pA004pU004pG004pC004 | | 04pU007pC004pU004pU004pG00 | |
| | | pA004pU004 | | 4pA004p001C004p001A004 | |
| 411 | 133 | A004p001A004p001G004pA004 | 817 | U004p001C007p001A004pU004p | 1058 |
| | | pC004pU004pU007pU004pU007 | | G004pC007pA004pU004pU004pC | |
| | | pU007pC007pG004pA004pA004 | | 004pG004pA004pA004pA007pA0 | |
| | | pU004pG004pC004pA004pU004 | | 04pA007pG004pU004pC004pU00 | |
| | | pG004pA004 | | 4pU004p001G004p001A004 | |
| 412 | 164 | G004p001C004p001C004pU004 | 818 | U004p001A007p001C004pU004p | 1059 |
| | | pC004pC004pC007pA004pU007 | | U004pC007pC004pC004pA004pG | |
| | | pC007pC007pC004pU004pG004 | | 004pG004pG004pA004pU007pG0 | |
| | | pG004pG004pA004pA004pG004 | | 04pG007pG004pA004pG004pG00 | |
| | | pU004pA004 | | 4pC004p001C004p001A004 | |
| 413 | 244 | C004p001A004p001A004pU004 | 819 | U004p001U007p001C004pC004p | 1060 |
| | | pA004pA004pG007pA004pU007 | | A004pA007pC004pC004pA004pC | |
| | | pC007pU007pG004pU004pG004 | | 004pA004pG004pA004pU007pC0 | |
| | | pG004pU004pU004pG004pG004 | | 04pU007pU004pA004pU004pU00 | |
| | | pA004pA004 | | 4pG004p001U004p001U004 | |
| 414 | 277 | A004p001A004p001A004pG004 | 820 | A004p001A007p001A004pA004p | 1061 |
| | | pU004pU004pU007pG004pA007 | | C004pA007pU004pC004pC004pU | |
| | | pA007pG007pA004pG004pG004 | | 004pC004pU004pU004pC007pA0 | |
| | | pA004pU004pG004pU004pU004 | | 04pA007pA004pC004pU004pU00 | |
| | | pU004pU004 | | 4pU004p001G004p001G004 | |
| 415 | 295 | U004p001U004p001U004pG004 | 821 | A004p001U007p001U004pA004p | 1062 |
| | | pA004pG004pC007pA004pG007 | | U004pA007pA004pU004pG004pU | |
| | | pU007pG007pA004pC004pA004 | | 004pC004pA004pC004pU007pG0 | |
| | | pU004pU004pA004pU004pA004 | | 04pC007pU004pC004pA004pA00 | |
| | | pA004pU004 | | 4pA004p001A004p001C004 | |
| 416 | 313 | A004p001A004p001U004pU004 | 822 | U004p001A007p001U004pC004p | 1063 |
| | | pC004pU004pG007pA004pC007 | | G004pU007pG004pU004pU004pA | |
| | | pA007pA007pU004pA004pA004 | | 004pU004pU004pG004pU007pC0 | |
| | | pC004pA004pC004pG004pA004 | | 04pA007pG004pA004pA004pU00 | |
| | | pU004pA004 | | 4pU004p001A004p001U004 | |
| 417 | 315 | U004p001U004p001C004pU004 | 823 | U004p001G007p001C004pA004p | 1064 |
| | | pG004pA004pC007pA004pA007 | | U004pC007pG004pU004pG004pU | |
| | | pU007pA007pA004pC004pA004 | | 004pU004pA004pU004pU007pG0 | |
| | | pC004pG004pA004pU004pG004 | | 04pU007pC004pA004pG004pA00 | |
| | | pC004pA004 | | 4pA004p001U004p001U004 | |
| 418 | 316 | U004p001C004p001U004pG004 | 824 | A004p001U007p001G004pC004p | 1065 |
| | | pA004pC004pA007pA004pU007 | | A004pU007pC004pG004pU004pG | |
| | | pA007pA007pC004pA004pC004 | | 004pU004pU004pA004pU007pU0 | |
| | | pG004pA004pU004pG004pC004 | | 04pG007pU004pC004pA004pG00 | |
| | | pA004pU004 | | 4pA004p001A004p001U004 | |
| 419 | 318 | U004p001G004p001A004pC004 | 825 | U004p001U007p001A004pU004p | 1066 |
| | | pA004pA004pU007pA004pA007 | | G004pC007pA004pU004pC004pG | |
| | | pC007pA007pC004pG004pA004 | | 004pU004pG004pU004pU007pA0 | |
| | | pU004pG004pC004pA004pU004 | | 04pU007pU004pG004pU004pC00 | |
| | | pA004pA004 | | 4pA004p001G004p001A004 | |
| 420 | 357 | U004p001C004p001C004pA004 | 826 | U004p001A007p001C004pG004p | 1067 |
| | | pG004pU004pU007pC004pC007 | | U004pA007pA004pA004pU004pU | |
| | | pA007pG007pA004pA004pU004 | | 004pC004pU004pG004pG007pA0 | |
| | | pU004pU004pA004pC004pG004 | | 04pA007pC004pU004pG004pG00 | |
| | | pU004pA004 | | 4pA004p001A004p001G004 | |
| 421 | 360 | A004p001G004p001U004pU004 | 827 | U004p001U007p001U004pG004p | 1068 |
| | | pC004pC004pA007pG004pA007 | | A004pC007pG004pU004pA004pA | |
| | | pA007pU007pU004pU004pA004 | | 004pA004pU004pU004pC007pU0 | |
| | | pc004pG004pU004pC004pA004 | | 04pG007pG004pA004pA004pC00 | |
| | | pA004pA004 | | 4pU004p001G004p001G004 | |
| 422 | 362 | U004p001U004p001C004pC004 | 828 | A004p001A007p001G004pU004p | 1069 |
| | | pA004pG004pA007pA004pU007 | | U004pG007pA004pC004pG004pU | |
| | | pU007pU007pA004pC004pG004 | | 004pA004pA004pA004pU007pU0 | |
| | | pU004pC004pA004pA004pC004 | | 04pC007pU004pG004pG004pA00 | |
| | | pU004pU004 | | 4pA004p001C004p001U004 | |
| 423 | 364 | C004p001C004p001A004pG004 | 829 | U004p001C007p001A004pA004p | 1070 |
| | | pA004pA004pU007pU004pU007 | | G004pU007pU004pG004pA004pC | |
| | | pA007pC007pG004pU004pC004 | | 004pG004pU004pA004pA007pA0 | |
| | | pA004pA004pC004pU004pU004 | | 04pU007pU004pC004pU004pG00 | |
| | | pG004pA004 | | 4pG004p001A004p001A004 | |
| 424 | 366 | A004p001G004p001A004pA004 | 830 | A004p001U007p001C004pC004p | 1071 |
| | | pU004pU004pU007pA004pC007 | | A004pA007pG004pU004pU004pG | |
| | | pG007pU007pC004pA004pA004 | | 004pA004pC004pG004pU007pA0 | |
| | | pC004pU004pU004pG004pG004 | | 04pA007pA004pU004pU004pC00 | |
| | | pA004pU004 | | 4pU004p001G004p001G004 | |
| 425 | 370 | U004p001U004p001U004pA004 | 831 | U004p001U007p001U004pG004p | 1072 |
| | | pC004pG004pU007pC004pA007 | | A004pU007pC004pC004pA004pA | |
| | | pA007pC007pU004pU004pG004 | | 004pG004pU004pU004pG007pA0 | |
| | | pG004pA004pU004pC004pA004 | | 04pC007pG004pU004pA004pA00 | |
| | | pA004pA004 | | 4pA004p001U004p001U004 | |
| 426 | 371 | U004p001U004p001A004pC004 | 832 | U004p001U007p001U004pU004p | 1073 |
| | | pG004pU004pC007pA004pA007 | | G004pA007pU004pC004pC004pA | |
| | | pC007pU007pU004pG004pG004 | | 004pA004pG004pU004pU007pG0 | |
| | | pA004pU004pC004pA004pA004 | | 04pA007pC004pG004pU004pA00 | |
| | | pA004pA004 | | 4pA004p001A004p001U004 | |
| 427 | 434 | U004p001U004p001U004pG004 | 833 | U004p001A007p001C004pA004p | 1074 |
| | | pU004pA004pU007pU004pC007 | | A004pC007pU004pG004pU004pA | |
| | | pA007pG007pU004pA004pC004 | | 004pC004pU004pG004pA007pA0 | |
| | | pA004pG004pU004pU004pG004 | | 04pU007pA004pC004pA004pA00 | |
| | | pU004pA004 | | 4pA004p001A004p001C004 | |
| 428 | 439 | A004p001U004p001U004pC004 | 834 | U004p001G007p001A004pA004p | 1075 |
| | | pA004pG004pU007pA004pC007 | | U004pG007pA004pC004pA004pA | |
| | | pA007pG007pU004pU004pG004 | | 004pC004pU004pG004pU007pA0 | |
| | | pU004pC004pA004pU004pU004 | | 04pC007pU004pG004pA004pA00 | |
| | | pC004pA004 | | 4pU004p001A004p001C004 | |
| 429 | 451 | U004p001G004p001U004pC004 | 835 | U004p001U007p001G004pU004p | 1076 |
| | | pA004pU004pU007pC004pA007 | | C004pU007pA004pA004pG004pA | |
| | | pC007pU007pU004pC004pU004 | | 004pA004pG004pU004pG007pA0 | |
| | | pU004pA004pG004pA004pC004 | | 04pA007pU004pG004pA004pC00 | |
| | | pA004pA004 | | 4pA004p001A004p001C004 | |
| 430 | 461 | U004p001U004p001C004pU004 | 836 | U004p001G007p001U004pC004p | 1077 |
| | | pU004pA004pG007pA004pC007 | | A004pA007pU004pU004pC004pU | |
| | | pA007pA007pA004pG004pA004 | | 004pU004pU004pG004pU007pC0 | |
| | | pA004pU004pU004pG004pA004 | | 04pU007pA004pA004pG004pA00 | |
| | | pC004pA004 | | 4pA004p001G004p001U004 | |
| 431 | 543 | U004p001A004p001G004pC004 | 837 | A004p001A007p001C004pU004p | 1078 |
| | | pA004pA004pA007pG004pU007 | | G004pA007pG004pG004pG004pC | |
| | | pU007pU007pG004pC004pC004 | | 004pA004pA004pA004pC007pU0 | |
| | | pC004pU004pC004pA004pG004 | | 04pU007pU004pG004pC004pU00 | |
| | | pU004pU004 | | 4pA004p001A004p001U004 | |
| 432 | 561 | G004p001U004p001U004pC004 | 838 | U004p001U007p001U004pC004p | 1079 |
| | | pC004pA004pA007pC004pU007 | | A004pU007pC004pC004pU004pG | |
| | | pC007pA007pC004pA004pG004 | | 004pU004pG004pA004pG007pU0 | |
| | | pG004pA004pU004pG004pA004 | | 04pU007pG004pG004pA004pA00 | |
| | | pA004pA004 | | 4pC004p001U004p001G004 | |
| 433 | 587 | G004p001A004p001A004pA004 | 839 | U004p001A007p001U004pU004p | 1080 |
| | | pA004pU004pA007pU004pU007 | | C004pC007pA004pC004pG004pA | |
| | | pG007pC007pU004pC004pG004 | | 004pG004pC004pA004pA007pU0 | |
| | | pU004pG004pG004pA004pA004 | | 04pA007pU004pU004pU004pU00 | |
| | | pU004pA004 | | 4pC004p001C004p001C004 | |
| 434 | 588 | A004p001A004p001A004pA004 | 840 | U004p001C007p001A004pU004p | 1081 |
| | | pU004pA004pU007pU004pG007 | | U004pC007pC004pA004pC004pG | |
| | | pC007pU007pC004pG004pU004 | | 004pA004pG004pC004pA007pA0 | |
| | | pG004pG004pA004pA004pU004 | | 04pU007pA004pU004pU004pU00 | |
| | | pG004pA004 | | 4pU004p001C004p001C004 | |
| 435 | 589 | A004p001A004p001A004pU004 | 841 | U004p001C007p001C004pA004p | 1082 |
| | | pA004pU004pU007pG004pC007 | | U004pU007pC004pC004pA004pC | |
| | | pU007pC007pG004pU004pG004 | | 004pG004pA004pG004pC007pA0 | |
| | | pG004pA004pA004pU004pG004 | | 04pA007pU004pA004pU004pU00 | |
| | | pG004pA004 | | 4pU004p001U004p001C004 | |
| 436 | 590 | A004p001A004p001U004pA004 | 842 | U004p001G007p001C004pC004p | 1083 |
| | | pU004pU004pG007pC004pU007 | | A004pU007pU004pC004pC004pA | |
| | | pC007pG007pU004pG004pG004 | | 004pC004pG004pA004pG007pC0 | |
| | | pA004pA004pU004pG004pG004 | | 04pA007pA004pU004pA004pU00 | |
| | | pC004pA004 | | 4pU004p001U004p001U004 | |
| 437 | 593 | A004p001U004p001U004pG004 | 843 | A004p001A007p001U004pU004p | 1084 |
| | | pC004pU004pC007pG004pU007 | | G004pC007pC004pA004pU004pU | |
| | | pG007pG007pA004pA004pU004 | | 004pC004pC004pA004pC007pG0 | |
| | | pG004pG004pC004pA004pA004 | | 04pA007pG004pC004pA004pA00 | |
| | | pU004pU004 | | 4pU004p001A004p001U004 | |
| 438 | 595 | U004p001G004p001C004pU004 | 844 | A004p001A007p001A004pA004p | 1085 |
| | | pC004pG004pU007pG004pG007 | | U004pU007pG004pC004pC004pA | |
| | | pA007pA007pU004pG004pG004 | | 004pU004pU004pC004pC007pA0 | |
| | | pC004pA004pA004pU004pU004 | | 04pC007pG004pA004pG004pC00 | |
| | | pU004pU004 | | 4pA004p001A004p001U004 | |
| 439 | 600 | G004p001U004p001G004pG004 | 845 | U004p001A007p001C004pC004p | 1086 |
| | | pA004pA004pU007pG004pG007 | | U004pA007pA004pA004pA004pU | |
| | | pC007pA007pA004pU004pU004 | | 004pU004pG004pC004pC007pA0 | |
| | | pU004pU004pA004pG004pG004 | | 04pU007pU004pC004pC004pA00 | |
| | | pU004pA004 | | 4pC004p001G004p001A004 | |
| 440 | 620 | C004p001C004p001U004pA004 | 846 | A004p001G007p001C004pA004p | 1087 |
| | | pc004pG004pU007pU004pU007 | | U004pC007pG004pA004pG004pG | |
| | | pA007pC007pC004pC004pU004 | | 004pG004pU004pA004pA007pA0 | |
| | | pC004pG004pA004pU004pG004 | | 04pC007pG004pU004pA004pG00 | |
| | | pC004pU004 | | 4pG004p001A004p001C004 | |
| 441 | 621 | C004p001U004p001A004pC004 | 847 | U004p001A007p001G004pC004p | 1088 |
| | | pG004pU004pU007pU004pA007 | | A004pU007pC004pG004pA004pG | |
| | | pC007pC007pC004pU004pC004 | | 004pG004pG004pU004pA007pA0 | |
| | | pG004pA004pU004pG004pC004 | | 04pA007pC004pG004pU004pA00 | |
| | | pU004pA004 | | 4pG004p001G004p001A004 | |
| 442 | 641 | C004p001U004p001U004pG004 | 848 | A004p001A007p001U004pC004p | 1089 |
| | | pU004pU004pG007pA004pA007 | | A004pC007pA004pA004pG004pA | |
| | | pU007pG007pU004pC004pU004 | | 004pC004pA004pU004pU007pC0 | |
| | | pU004pG004pU004pG004pA004 | | 04pA007pA004pC004pA004pA00 | |
| | | pU004pU004 | | 4pG004p001A004p001G004 | |
| 443 | 644 | G004p001U004p001U004pG004 | 849 | U004p001C007p001C004pA004p | 1090 |
| | | pA004pA004pU007pG004pU007 | | A004pU007pC004pA004pC004pA | |
| | | pc007pU007pU004pG004pU004 | | 004pA004pG004pA004pC007pA0 | |
| | | pG004pA004pU004pU004pG004 | | 04pU007pU004pC004pA004pA00 | |
| | | pG004pA004 | | 4pC004p001A004p001A004 | |
| 444 | 683 | G004p001U004p001A004pC004 | 850 | U004p001A007p001U004pA004p | 1091 |
| | | pG004pU004pC007pA004pG007 | | A004pU007pU004pU004pC004pA | |
| | | pC007pU007pU004pG004pA004 | | 004pA004pG004pC004pU007pG0 | |
| | | pA004pA004pU004pU004pA004 | | 04pA007pC004pG004pU004pA00 | |
| | | pU004pA004 | | 4pC004p001C004p001C004 | |
| 445 | 688 | U004p001C004p001A004pG004 | 851 | U004p001A007p001G004pC004p | 1092 |
| | | pC004pU004pU007pG004pA007 | | A004pC007pA004pU004pA004pA | |
| | | pA007pA007pU004pU004pA004 | | 004pU004pU004pU004pC007pA0 | |
| | | pU004pG004pU004pG004pC004 | | 04pA007pG004pC004pU004pG00 | |
| | | pU004pA004 | | 4pA004p001C004p001G004 | |
| 446 | 697 | A004p001A004p001U004pU004 | 852 | U004p001A007p001G004pC004p | 1093 |
| | | pA004pU004pG007pU004pG007 | | C004pA007pA004pA004pG004pC | |
| | | pC007pU007pG004pC004pU004 | | 004pA004pG004pC004pA007pC0 | |
| | | pU004pU004pG004pG004pC004 | | 04pA007pU004pA004pA004pU00 | |
| | | pU004pA004 | | 4pU004p001U004p001C004 | |
| 447 | 710 | U004p001U004p001U004pG004 | 853 | A004p001A007p001G004pA004p | 1094 |
| | | pG004pC004pU007pG004pC007 | | A004pC007pU004pG004pA004pC | |
| | | pA007pU007pG004pU004pC004 | | 004pA004pU004pG004pC007pA0 | |
| | | pA004pG004pU004pU004pC004 | | 04pG007pC004pC004pA004pA00 | |
| | | pU004pU004 | | 4pA004p001G004p001C004 | |
| 448 | 729 | U004p001U004p001G004pC004 | 854 | U004p001G007p001A004pA004p | 1095 |
| | | pC004pA004pA007pC004pU007 | | C004pA007pC004pG004pA004pA | |
| | | pA007pC007pU004pU004pC004 | | 004pG004pU004pA004pG007pU0 | |
| | | pG004pU004pG004pU004pU004 | | 04pU007pG004pG004pC004pA00 | |
| | | pC004pA004 | | 4pA004p001G004p001A004 | |
| 449 | 730 | U004p001G004p001C004pC004 | 855 | A004p001U007p001G004pA004p | 1096 |
| | | pA004pA004pC007pU004pA007 | | A004pC007pA004pC004pG004pA | |
| | | pC007pU007pU004pC004pG004 | | 004pA004pG004pU004pA007pG0 | |
| | | pU004pG004pU004pU004pC004 | | 04pU007pU004pG004pG004pC00 | |
| | | pA004pU004 | | 4pA004p001A004p001G004 | |
| 450 | 734 | A004p001A004p001C004pU004 | 856 | A004p001G007p001U004pC004p | 1097 |
| | | pA004pC004pU007pU004pC007 | | A004pU007pG004pA004pA004pC | |
| | | pG007pU007pG004pU004pU004 | | 004pA004pC004pG004pA007pA0 | |
| | | pC004pA004pU004pG004pA004 | | 04pG007pU004pA004pG004pU00 | |
| | | pC004pU004 | | 4pU004p001G004p001G004 | |
| 451 | 738 | A004p001C004p001U004pU004 | 857 | A004p001G007p001A004pA004p | 1098 |
| | | pC004pG004pU007pG004pU007 | | A004pG007pU004pC004pA004pU | |
| | | pU007pC007pA004pU004pG004 | | 004pG004pA004pA004pC007pA0 | |
| | | pA004pC004pU004pU004pU004 | | 04pC007pG004pA004pA004pG00 | |
| | | pC004pU004 | | 4pU004p001A004p001G004 | |
| 452 | 739 | C004p001U004p001U004pC004 | 858 | A004p001A007p001G004pA004p | 1099 |
| | | pG004pU004pG007pU004pU007 | | A004pA007pG004pU004pC004pA | |
| | | pC007pA007pU004pG004pA004 | | 004pU004pG004pA004pA007pC0 | |
| | | pC004pU004pU004pU004pC004 | | 04pA007pC004pG004pA004pA00 | |
| | | pU004pU004 | | 4pG004p001U004p001A004 | |
| 453 | 902 | A004p001U004p001G004pU004 | 859 | A004p001A007p001G004pA004p | 1100 |
| | | pC004pU004pC007pU004pA007 | | A004pC007pC004pA004pA004pG | |
| | | pG007pG007pC004pU004pU004 | | 004pC004pC004pU004pA007pG0 | |
| | | pG004pG004pU004pU004pC004 | | 04pA007pG004pA004pC004pA00 | |
| | | pU004pU004 | | 4pU004p001A004p001A004 | |
| 454 | 909 | U004p001A004p001G004pG004 | 860 | U004p001A007p001U004pG004p | 1101 |
| | | pC004pU004pU007pG004pG007 | | A004pA007pC004pA004pA004pG | |
| | | pU007pU007pC004pU004pU004 | | 004pA004pA004pC004pC007pA0 | |
| | | pG004pU004pU004pC004pA004 | | 04pA007pG004pC004pC004pU00 | |
| | | pU004pA004 | | 4pA004p001G004p001A004 | |
| 455 | 919 | U004p001C004p001U004pU004 | 861 | U004p001G007p001A004pC004p | 1102 |
| | | pG004pU004pU007pC004pA007 | | U004pG007pU004pG004pA004pG | |
| | | pU007pG007pC004pU004pC004 | | 004pC004pA004pU004pG007pA0 | |
| | | pA004pC004pA004pG004pU004 | | 04pA007pC004pA004pA004pG00 | |
| | | pC004pA004 | | 4pA004p001A004p001C004 | |
| 456 | 927 | A004p001U004p001G004pC004 | 862 | U004p001U007p001A004pU004p | 1103 |
| | | pU004pC004pA007pC004pA007 | | C004pC007pA004pG004pC004pG | |
| | | pG007pU007pC004pG004pC004 | | 004pA004pC004pU004pG007pU0 | |
| | | pU004pG004pG004pA004pU004 | | 04pG007pA004pG004pC004pA00 | |
| | | pA004pA004 | | 4pU004p001G004p001A004 | |
| 457 | 928 | U004p001G004p001C004pU004 | 863 | U004p001C007p001U004pA004p | 1104 |
| | | pC004pA004pC007pA004pG007 | | U004pC007pC004pA004pG004pC | |
| | | pU007pC007pG004pC004pU004 | | 004pG004pA004pC004pU007pG0 | |
| | | pG004pG004pA004pU004pA004 | | 04pU007pG004pA004pG004pC00 | |
| | | pG004pA004 | | 4pA004p001U004p001G004 | |
| 458 | 932 | C004p001A004p001C004pA004 | 864 | A004p001U007p001C004pA004p | 1105 |
| | | pG004pU004pC007pG004pC007 | | G004pC007pU004pA004pU004pC | |
| | | pU007pG007pG004pA004pU004 | | 004pC004pA004pG004pC007pG0 | |
| | | pA004pG004pC004pU004pG004 | | 04pA007pC004pU004pG004pU00 | |
| | | pA004pU004 | | 4pG004p001A004p001G004 | |
| 459 | 935 | A004p001G004p001U004pC004 | 865 | A004p001G007p001G004pA004p | 1106 |
| | | pG004pC004pU007pG004pG007 | | U004pC007pA004pG004pC004pU | |
| | | pA007pU007pA004pG004pC004 | | 004pA004pU004pC004pC007pA0 | |
| | | pU004pG004pA004pU004pC004 | | 04pG007pC004pG004pA004pC00 | |
| | | pC004pU004 | | 4pU004p001G004p001U004 | |
| 460 | 954 | C004p001U004p001U004pC004 | 866 | U004p001U007p001G004pU004p | 1107 |
| | | pU004pC004pC007pU004pC007 | | A004pC007pU004pG004pU004pU | |
| | | pA007pA007pA004pA004pC004 | | 004pU004pU004pG004pA007pG0 | |
| | | pA004pG004pU004pA004pC004 | | 04pG007pA004pG004pA004pA00 | |
| | | pA004pA004 | | 4pG004p001G004p001A004 | |
| 461 | 957 | C004p001U004p001C004pC004 | 867 | U004p001U007p001G004pC004p | 1108 |
| | | pU004pC004pA007pA004pA007 | | U004pG007pU004pA004pC004pU | |
| | | pA007pC007pA004pG004pU004 | | 004pG004pU004pU004pU007pU0 | |
| | | pA004pC004pA004pG004pC004 | | 04pG007pA004pG004pG004pA00 | |
| | | pA004pA004 | | 4pG004p001A004p001A004 | |
| 462 | 966 | A004p001C004p001A004pG004 | 868 | U004p001A007p001G004pA004p | 1109 |
| | | pU004pA004pC007pA004pG007 | | A004pG007pU004pA004pU004pC | |
| | | pC007pA007pG004pA004pU004 | | 004pU004pG004pC004pU007pG0 | |
| | | pA004pC004pU004pU004pC004 | | 04pU007pA004pC004pU004pG00 | |
| | | pU004pA004 | | 4pU004p001U004p001U004 | |
| 463 | 967 | C004p001A004p001G004pU004 | 869 | U004p001U007p001A004pG004p | 1110 |
| | | pA004pC004pA007pG004pC007 | | A004pA007pG004pU004pA004pU | |
| | | pA007pG007pA004pU004pA004 | | 004pC004pU004pG004pC007pU0 | |
| | | pC004pU004pU004pC004pU004 | | 04pG007pU004pA004pC004pU00 | |
| | | pA004pA004 | | 4pG004p001U004p001U004 | |
| 464 | 968 | A004p001G004p001U004pA004 | 870 | U004p001U007p001U004pA004p | 1111 |
| | | pC004pA004pG007pC004pA007 | | G004pA007pA004pG004pU004pA | |
| | | pG007pA007pU004pA004pC004 | | 004pU004pC004pU004pG007pC0 | |
| | | pU004pU004pC004pU004pA004 | | 04pU007pG004pU004pA004pC00 | |
| | | pA004pA004 | | 4pU004p001G004p001U004 | |
| 465 | 972 | C004p001A004p001G004pC004 | 871 | A004p001A007p001A004pC004p | 1112 |
| | | pA004pG004pA007pU004pA007 | | C004pU007pU004pA004pG004pA | |
| | | pC007pU007pU004pC004pU004 | | 004pA004pG004pU004pA007pU0 | |
| | | pA004pA004pG004pG004pU004 | | 04pC007pU004pG004pC004pU00 | |
| | | pU004pU004 | | 4pG004p001U004p001A004 | |
| 466 | 1083 | U004p001U004p001G004pA004 | 872 | U004p001U007p001A004pG004p | 1113 |
| | | pA004pC004pA007pA004pG007 | | G004pG007pU004pA004pA004pU | |
| | | pU007pU007pA004pU004pU004 | | 004pA004pA004pC004pU007pU0 | |
| | | pA004pC004pC004pC004pU004 | | 04pG007pU004pU004pC004pA00 | |
| | | pA004pA004 | | 4pA004p001U004p001A004 | |
| 467 | 1085 | G004p001A004p001A004pC004 | 873 | A004p001C007p001U004pU004p | 1114 |
| | | pA004pA004pG007pU004pU007 | | A004pG007pG004pG004pU004pA | |
| | | pA007pU007pU004pA004pC004 | | 004pA004pU004pA004pA007pC0 | |
| | | pC004pC004pU004pA004pA004 | | 04pU007pU004pG004pU004pU00 | |
| | | pG004pU004 | | 4pC004p001A004p001A004 | |
| 468 | 1087 | A004p001C004p001A004pA004 | 874 | A004p001A007p001A004pC004p | 1115 |
| | | pG004pU004pU007pA004pU007 | | U004pU007pA004pG004pG004pG | |
| | | pU007pA007pC004pC004pC004 | | 004pU004pA004pA004pU007pA0 | |
| | | pU004pA004pA004pG004pU004 | | 04pA007pC004pU004pU004pG00 | |
| | | pU004pU004 | | 4pU004p001U004p001C004 | |
| 469 | 1088 | C004p001A004p001A004pG004 | 875 | U004p001A007p001A004pA004p | 1116 |
| | | pU004pU004pA007pU004pU007 | | C004pU007pU004pA004pG004pG | |
| | | pA007pC007pC004pC004pU004 | | 004pG004pU004pA004pA007pU0 | |
| | | pA004pA004pG004pU004pU004 | | 04pA007pA004pC004pU004pU00 | |
| | | pU004pA004 | | 4pG004p001U004p001U004 | |
| 470 | 1090 | A004p001G004p001U004pU004 | 876 | U004p001C007p001U004pA004p | 1117 |
| | | pA004pU004pU007pA004pC007 | | A004pA007pC004pU004pU004pA | |
| | | pC007pC007pU004pA004pA004 | | 004pG004pG004pG004pU007pA0 | |
| | | pG004pU004pU004pU004pA004 | | 04pA007pU004pA004pA004pC00 | |
| | | pG004pA004 | | 4pU004p001U004p001G004 | |
| 471 | 1112 | C004p001U004p001C004pC004 | 877 | U004p001U007p001A004pC004p | 1118 |
| | | pU004pU004pC007pU004pG007 | | U004pU007pG004pA004pC004pA | |
| | | pG007pC007pU004pG004pU004 | | 004pG004pC004pC004pA007pG0 | |
| | | pC004pA004pA004pG004pU004 | | 04pA007pA004pG004pG004pA00 | |
| | | pA004pA004 | | 4pG004p001A004p001G004 | |
| 472 | 1172 | U004p001U004p001A004pA004 | 878 | A004p001G007p001A004pU004p | 1119 |
| | | pA004pA004pA007pA004pC007 | | G004pU007pG004pA004pU004pA | |
| | | pC007pC007pU004pA004pU004 | | 004pG004pG004pG004pU007pU0 | |
| | | pC004pA004pC004pA004pU004 | | 04pU007pU004pU004pU004pA00 | |
| | | pC004pU004 | | 4pA004p001U004p001G004 | |
| 473 | 1181 | C004p001C004p001U004pA004 | 879 | U004p001A007p001C004pU004p | 1120 |
| | | pU004pC004pA007pC004pA007 | | A004pC007pA004pG004pG004pA | |
| | | pU007pC007pU004pC004pC004 | | 004pG004pA004pU004pG007pU0 | |
| | | pU004pG004pU004pA004pG004 | | 04pG007pA004pU004pA004pG00 | |
| | | pU004pA004 | | 4pG004p001G004p001U004 | |
| 474 | 1183 | U004p001A004p001U004pC004 | 880 | U004p001U007p001C004pA004p | 1121 |
| | | pA004pC004pA007pU004pC007 | | C004pU007pA004pC004pA004pG | |
| | | pU007pC007pC004pU004pG004 | | 004pG004pA004pG004pA007pU0 | |
| | | pU004pA004pG004pU004pG004 | | 04pG007pU004pG004pA004pU00 | |
| | | pA004pA004 | | 4pA004p001G004p001G004 | |
| 475 | 1224 | A004p001U004p001U004pG004 | 881 | U004p001A007p001G004pG004p | 1122 |
| | | pU004pU004pG007pU004pA007 | | U004pU007pC004pA004pC004pG | |
| | | pG007pA007pC004pG004pU004 | | 004pU004pC004pU004pA007pC0 | |
| | | pG004pA004pA004pC004pC004 | | 04pA007pA004pC004pA004pA00 | |
| | | pU004pA004 | | 4pU004p001U004p001G004 | |
| 476 | 1283 | G004p001A004p001A004pG004 | 882 | U004p001C007p001G004pG004p | 1123 |
| | | pA004pG004pA007pC004pA007 | | U004pU007pU004pA004pU004pC | |
| | | pG007pG007pG004pA004pU004 | | 004pC004pC004pU004pG007pU0 | |
| | | pA004pA004pA004pC004pC004 | | 04pC007pU004pC004pU004pU00 | |
| | | pG004pA004 | | 4pC004p001C004p001U004 | |
| 477 | 1284 | A004p001A004p001G004pA004 | 883 | U004p001U007p001C004pG004p | 1124 |
| | | pG004pA004pC007pA004pG007 | | G004pU007pU004pU004pA004pU | |
| | | pG007pG007pA004pU004pA004 | | 004pC004pC004pC004pU007pG0 | |
| | | pA004pA004pC004pC004pG004 | | 04pU007pC004pU004pC004pU00 | |
| | | pA004pA004 | | 4pU004p001C004p001C004 | |
| 478 | 1287 | A004p001G004p001A004pC004 | 884 | U004p001U007p001U004pC004p | 1125 |
| | | pA004pG004pG007pG004pA007 | | U004pC007pG004pG004pU004pU | |
| | | pU007pA007pA004pA004pC004 | | 004pU004pA004pU004pC007pC0 | |
| | | pC004pG004pA004pG004pA004 | | 04pC007pU004pG004pU004pC00 | |
| | | pA004pA004 | | 4pU004p001C004p001U004 | |
| 479 | 1288 | G004p001A004p001C004pA004 | 885 | U004p001U007p001U004pU004p | 1126 |
| | | pG004pG004pG007pA004pU007 | | C004pU007pC004pG004pG004pU | |
| | | pA007pA007pA004pC004pC004 | | 004pU004pU004pA004pU007pC0 | |
| | | pG004pA004pG004pA004pA004 | | 04pC007pC004pU004pG004pU00 | |
| | | pA004pA004 | | 4pC004p001U004p001C004 | |
| 480 | 1291 | A004p001G004p001G004pG004 | 886 | U004p001U007p001U004pC004p | 1127 |
| | | pA004pU004pA007pA004pA007 | | U004pU007pU004pC004pU004pC | |
| | | pC007pC007pG004pA004pG004 | | 004pG004pG004pU004pU007pU0 | |
| | | pA004pA004pA004pG004pA004 | | 04pA007pU004pC004pC004pC00 | |
| | | pA004pA004 | | 4pU004p001G004p001U004 | |
| 481 | 1297 | A004p001A004p001A004pC004 | 887 | U004p001C007p001A004pA004p | 1128 |
| | | pC004pG004pA007pG004pA007 | | C004pU007pU004pU004pU004pC | |
| | | pA007pA007pG004pA004pA004 | | 004pU004pU004pU004pC007pU0 | |
| | | pA004pA004pG004pU004pU004 | | 04pC007pG004pG004pU004pU00 | |
| | | pG004pA004 | | 4pU004p001A004p001U004 | |
| 482 | 1313 | G004p001U004p001U004pG004 | 888 | U004p001A007p001A004pG004p | 1129 |
| | | pA004pG004pG007pU004pU007 | | G004pG007pU004pU004pU004pU | |
| | | pA007pU007pA004pA004pA004 | | 004pA004pU004pA004pA007pC0 | |
| | | pA004pC004pC004pC004pU004 | | 04pC007pU004pC004pA004pA00 | |
| | | pU004pA004 | | 4pC004p001U004p001U004 | |
| 483 | 1318 | G004p001G004p001U004pU004 | 889 | U004p001C007p001C004pA004p | 1130 |
| | | pA004pU004pA007pA004pA007 | | C004pU007pA004pA004pG004pG | |
| | | pA007pC007pC004pC004pU004 | | 004pG004pU004pU004pU007pU0 | |
| | | pU004pA004pG004pU004pG004 | | 04pA007pU004pA004pA004pC00 | |
| | | pG004pA004 | | 4pC004p001U004p001C004 | |
| 484 | 1326 | A004p001A004p001C004pC004 | 890 | U004p001U007p001G004pU004p | 1131 |
| | | pC004pU004pU007pA004pG007 | | U004pU007pC004pA004pG004pC | |
| | | pU007pG007pG004pC004pU004 | | 004pC004pA004pC004pU007pA0 | |
| | | pG004pA004pA004pA004pC004 | | 04pA007pG004pG004pG004pU00 | |
| | | pA004pA004 | | 4pU004p001U004p001U004 | |
| 485 | 1327 | A004p001C004p001C004pC004 | 891 | U004p001C007p001U004pG004p | 1132 |
| | | pU004pU004pA007pG004pU007 | | U004pU007pU004pC004pA004pG | |
| | | pG007pG007pC004pU004pG004 | | 004pC004pC004pA004pC007pU0 | |
| | | pA004pA004pA004pC004pA004 | | 04pA007pA004pG004pG004pG00 | |
| | | pG004pA004 | | 4pU004p001U004p001U004 | |
| 486 | 1328 | C004p001C004p001C004pU004 | 892 | A004p001U007p001C004pU004p | 1133 |
| | | pU004pA004pG007pU004pG007 | | G004pU007pU004pU004pC004pA | |
| | | pG007pC007pU004pG004pA004 | | 004pG004pC004pC004pA007pC0 | |
| | | pA004pA004pC004pA004pG004 | | 04pU007pA004pA004pG004pG00 | |
| | | pA004pU004 | | 4pG004p001U004p001U004 | |
| 487 | 1329 | C004p001C004p001U004pU004 | 893 | U004p001A007p001U004pC004p | 1134 |
| | | pA004pG004pU007pG004pG007 | | U004pG007pU004pU004pU004pC | |
| | | pC007pU007pG004pA004pA004 | | 004pA004pG004pC004pC007pA0 | |
| | | pA004pC004pA004pG004pA004 | | 04pC007pU004pA004pA004pG00 | |
| | | pU004pA004 | | 4pG004p001G004p001U004 | |
| 488 | 1357 | C004p001A004p001G004pA004 | 894 | U004p001C007p001A004pA004p | 1135 |
| | | pG004pC004pU007pA004pC007 | | C004pC007pA004pC004pA004pA | |
| | | pA007pU007pU004pU004pG004 | | 004pA004pU004pG004pU007pA0 | |
| | | pU004pG004pG004pU004pU004 | | 04pG007pC004pU004pC004pU00 | |
| | | pG004pA004 | | 4pG004p001U004p001U004 | |
| 489 | 1380 | A004p001C004p001U004pC004 | 895 | A004p001A007p001G004pU004p | 1136 |
| | | pC004pU004pC007pC004pU007 | | A004pU007pC004pG004pA004pG | |
| | | pU007pA007pC004pU004pC004 | | 004pU004pA004pA004pG007pG0 | |
| | | pG004pA004pU004pA004pC004 | | 04pA007pG004pG004pA004pG00 | |
| | | pU004pU004 | | 4pU004p001U004p001A004 | |
| 490 | 1381 | C004p001U004p001C004pC004 | 896 | U004p001A007p001A004pG004p | 1137 |
| | | pU004pC004pC007pU004pU007 | | U004pA007pU004pC004pG004pA | |
| | | pA007pC007pU004pC004pG004 | | 004pG004pU004pA004pA007pG0 | |
| | | pA004pU004pA004pC004pU004 | | 04pG007pA004pG004pG004pA00 | |
| | | pU004pA004 | | 4pG004p001U004p001U004 | |
| 491 | 1410 | U004p001G004p001G004pU004 | 897 | U004p001U007p001U004pC004p | 1138 |
| | | pG004pA004pC007pA004pC007 | | A004pG007pG004pU004pU004pC | |
| | | pA007pG007pG004pA004pA004 | | 004pC004pU004pG004pU007pG0 | |
| | | pC004pC004pU004pG004pA004 | | 04pU007pC004pA004pC004pC00 | |
| | | pA004pA004 | | 4pA004p001G004p001U004 | |
| 492 | 1494 | A004p001A004p001G004pG004 | 898 | U004p001A007p001C004pU004p | 1139 |
| | | pU004pG004pC007pA004pA007 | | A004pA007pG004pG004pA004pA | |
| | | pA007pA007pU004pU004pC004 | | 004pU004pU004pU004pU007pG0 | |
| | | pc004pU004pU004pA004pG004 | | 04pC007pA004pC004pC004pU00 | |
| | | pU004pA004 | | 4pU004p001U004p001C004 | |
| 493 | 1505 | U004p001U004p001C004pC004 | 899 | U004p001A007p001U004pC004p | 1140 |
| | | pU004pU004pA007pG004pU007 | | U004pC007pA004pG004pC004pA | |
| | | pG007pA007pU004pG004pC004 | | 004pU004pC004pA004pC007pU0 | |
| | | pU004pG004pA004pG004pA004 | | 04pA007pA004pG004pG004pA00 | |
| | | pU004pA004 | | 4pA004p001U004p001U004 | |
| 494 | 1516 | U004p001G004p001C004pU004 | 900 | A004p001C007p001U004pA004p | 1141 |
| | | pG004pA004pG007pA004pU007 | | A004pC007pU004pG004pG004pA | |
| | | pC007pA007pU004pC004pC004 | | 004pU004pG004pA004pU007pC0 | |
| | | pA004pG004pU004pU004pA004 | | 04pU007pC004pA004pG004pC00 | |
| | | pG004pU004 | | 4pA004p001U004p001C004 | |
| 495 | 1516 | U004p001G004p001C004pU004 | 901 | A004p001C007p001U004pA004p | 1142 |
| | | pG004pA004pG007pA004pU007 | | A004pC007pU004pG004pG004pA | |
| | | pC007pA007pU004pC004pC004 | | 004pU004pG004pA004pU007pC0 | |
| | | pA004pG004pU004pU004pA004 | | 04pU007pC004pA004pG004pC00 | |
| | | pG004pU004 | | 4pA004p001U004p001C004 | |
| 496 | 1519 | U004p001G004p001A004pG004 | 902 | U004p001U007p001G004pA004p | 1143 |
| | | pA004pU004pC007pA004pU007 | | C004pU007pA004pA004pC004pU | |
| | | pC007pC007pA004pG004pU004 | | 004pG004pG004pA004pU007pG0 | |
| | | pU004pA004pG004pU004pC004 | | 04pA007pU004pC004pU004pC00 | |
| | | pA004pA004 | | 4pA004p001G004p001C004 | |
| 497 | 1521 | A004p001G004p001A004pU004 | 903 | U004p001A007p001U004pU004p | 1144 |
| | | pC004pA004pU007pC004pC007 | | G004pA007pC004pU004pA004pA | |
| | | pA007pG007pU004pU004pA004 | | 004pC004pU004pG004pG007pA0 | |
| | | pG004pU004pC004pA004pA004 | | 04pU007pG004pA004pU004pC00 | |
| | | pU004pA004 | | 4pU004p001C004p001A004 | |
| 498 | 1523 | A004p001U004p001C004pA004 | 904 | A004p001G007p001C004pA004p | 1145 |
| | | pU004pC004pC007pA004pG007 | | U004pU007pG004pA004pC004pU | |
| | | pU007pU007pA004pG004pU004 | | 004pA004pA004pC004pU007pG0 | |
| | | pC004pA004pA004pU004pG004 | | 04pG007pA004pU004pG004pA00 | |
| | | pC004pU004 | | 4pU004p001C004p001U004 | |
| 499 | 1527 | U004p001C004p001C004pA004 | 905 | U004p001C007p001U004pU004p | 1146 |
| | | pG004pU004pU007pA004pG007 | | A004pG007pC004pA004pU004pU | |
| | | pU007pC007pA004pA004pU004 | | 004pG004pA004pC004pU007pA0 | |
| | | pG004pC004pU004pA004pA004 | | 04pA007pC004pU004pG004pG00 | |
| | | pG004pA004 | | 4pA004p001U004p001G004 | |
| 500 | 1530 | A004p001G004p001U004pU004 | 906 | U004p001A007p001U004pG004p | 1147 |
| | | pA004pG004pU007pC004pA007 | | C004pU007pU004pA004pG004pC | |
| | | pA007pU007pG004pC004pU004 | | 004pA004pU004pU004pG007pA0 | |
| | | pA004pA004pG004pC004pA004 | | 04pC007pU004pA004pA004pC00 | |
| | | pU004pA004 | | 4pU004p001G004p001G004 | |
| 501 | 1531 | G004p001U004p001U004pA004 | 907 | A004p001U007p001A004pU004p | 1148 |
| | | pG004pU004pC007pA004pA007 | | G004pC007pU004pU004pA004pG | |
| | | pU007pG007pC004pU004pA004 | | 004pC004pA004pU004pU007pG0 | |
| | | pA004pG004pC004pA004pU004 | | 04pA007pC004pU004pA004pA00 | |
| | | pA004pU004 | | 4pC004p001U004p001G004 | |
| 502 | 1532 | U004p001U004p001A004pG004 | 908 | U004p001A007p001U004pA004p | 1149 |
| | | pU004pC004pA007pA004pU007 | | U004pG007pC004pU004pU004pA | |
| | | pG007pC007pU004pA004pA004 | | 004pG004pC004pA004pU007pU0 | |
| | | pG004pC004pA004pU004pA004 | | 04pG007pA004pC004pU004pA00 | |
| | | pU004pA004 | | 4pA004p001C004p001U004 | |
| 503 | 1535 | G004p001U004p001C004pA004 | 909 | U004p001G007p001G004pG004p | 1150 |
| | | pA004pU004pG007pC004pU007 | | A004pU007pA004pU004pG004pC | |
| | | pA007pA007pG004pC004pA004 | | 004pU004pU004pA004pG007pC0 | |
| | | pU004pA004pU004pC004pC004 | | 04pA007pU004pU004pG004pA00 | |
| | | pC004pA004 | | 4pC004p001U004p001A004 | |
| 504 | 1546 | G004p001C004p001A004pU004 | 910 | A004p001A007p001C004pU004p | 1151 |
| | | pA004pU004pC007pC004pC007 | | U004pG007pU004pA004pG004pG | |
| | | pA007pG007pC004pC004pU004 | | 004pC004pU004pG004pG007pG0 | |
| | | pA004pC004pA004pA004pG004 | | 04pA007pU004pA004pU004pG00 | |
| | | pU004pU004 | | 4pC004p001U004p001U004 | |
| 505 | 1547 | C004p001A004p001U004pA004 | 911 | U004p001A007p001A004pC004p | 1152 |
| | | pU004pC004pC007pC004pA007 | | U004pU007pG004pU004pA004pG | |
| | | pG007pC007pC004pU004pA004 | | 004pG004pC004pU004pG007pG0 | |
| | | pC004pA004pA004pG004pU004 | | 04pG007pA004pU004pA004pU00 | |
| | | pU004pA004 | | 4pG004p001C004p001U004 | |
| 506 | 1548 | A004p001U004p001A004pU004 | 912 | U004p001C007p001A004pA004p | 1153 |
| | | pC004pC004pC007pA004pG007 | | C004pU007pU004pG004pU004pA | |
| | | pC007pC007pU004pA004pC004 | | 004pG004pG004pC004pU007pG0 | |
| | | pA004pA004pG004pU004pU004 | | 04pG007pG004pA004pU004pA00 | |
| | | pG004pA004 | | 4pU004p001G004p001C004 | |
| 507 | 1555 | A004p001G004p001C004pC004 | 913 | A004p001G007p001A004pG004p | 1154 |
| | | pU004pA004pC007pA004pA007 | | U004pU007pU004pC004pC004pA | |
| | | pG007pU007pU004pG004pG004 | | 004pA004pC004pU004pU007pG0 | |
| | | pA004pA004pA004pC004pU004 | | 04pU007pA004pG004pG004pC00 | |
| | | pC004pU004 | | 4pU004p001G004p001G004 | |
| 508 | 1561 | C004p001A004p001A004pG004 | 914 | U004p001C007p001C004pA004p | 1155 |
| | | pU004pU004pG007pG004pA007 | | U004pC007pA004pG004pA004pG | |
| | | pA007pA007pC004pU004pC004 | | 004pU004pU004pU004pC007pC0 | |
| | | pU004pG004pA004pU004pG004 | | 04pA007pA004pC004pU004pU00 | |
| | | pG004pA004 | | 4pG004p001U004p001A004 | |
| 509 | 1562 | A004p001A004p001G004pU004 | 915 | U004p001U007p001C004pC004p | 1156 |
| | | pU004pG004pG007pA004pA007 | | A004pU007pC004pA004pG004pA | |
| | | pA007pC007pU004pC004pU004 | | 004pG004pU004pU004pU007pC0 | |
| | | pG004pA004pU004pG004pG004 | | 04pC007pA004pA004pC004pU00 | |
| | | pA004pA004 | | 4pU004p001G004p001U004 | |
| 510 | 1567 | G004p001G004p001A004pA004 | 916 | U004p001G007p001A004pG004p | 1157 |
| | | pA004pC004pU007pC004pU007 | | U004pU007pU004pC004pC004pA | |
| | | pG007pA007pU004pG004pG004 | | 004pU004pC004pA004pG007pA0 | |
| | | pA004pA004pA004pC004pU004 | | 04pG007pU004pU004pU004pC00 | |
| | | pC004pA004 | | 4pC004p001A004p001A004 | |
| 511 | 1580 | G004p001A004p001A004pA004 | 917 | U004p001A007p001C004pA004p | 1158 |
| | | pC004pU004pC007pA004pU007 | | C004pC007pA004pC004pG004pC | |
| | | pG007pA007pG004pC004pG004 | | 004pU004pC004pA004pU007pG0 | |
| | | pU004pG004pG004pU004pG004 | | 04pA007pG004pU004pU004pU00 | |
| | | pU004pA004 | | 4pC004p001C004p001A004 | |
| 512 | 1582 | A004p001A004p001C004pU004 | 918 | U004p001A007p001U004pA004p | 1159 |
| | | pC004pA004pU007pG004pA007 | | C004pA007pC004pC004pA004pC | |
| | | pG007pC007pG004pU004pG004 | | 004pG004pC004pU004pC007pA0 | |
| | | pG004pU004pG004pU004pA004 | | 04pU007pG004pA004pG004pU00 | |
| | | pU004pA004 | | 4pU004p001U004p001C004 | |
| 513 | 1583 | A004p001C004p001U004pC004 | 919 | A004p001G007p001A004pU004p | 1160 |
| | | pA004pU004pG007pA004pG007 | | A004pC007pA004pC004pC004pA | |
| | | pC007pG007pU004pG004pG004 | | 004pC004pG004pC004pU007pC0 | |
| | | pU004pG004pU004pA004pU004 | | 04pA007pU004pG004pA004pG00 | |
| | | pC004pU004 | | 4pU004p001U004p001U004 | |
| 514 | 1585 | U004p001C004p001A004pU004 | 920 | A004p001U007p001A004pG004p | 1161 |
| | | pG004pA004pG007pC004pG007 | | A004pU007pA004pC004pA004pC | |
| | | pU007pG007pG004pU004pG004 | | 004pC004pA004pC004pG007pC0 | |
| | | pU004pA004pU004pC004pU004 | | 04pU007pC004pA004pU004pG00 | |
| | | pA004pU004 | | 4pA004p001G004p001U004 | |
| 515 | 1587 | A004p001U004p001G004pA004 | 921 | U004p001A007p001A004pU004p | 1162 |
| | | pG004pC004pG007pU004pG007 | | A004pG007pA004pU004pA004pC | |
| | | pG007pU007pG004pU004pA004 | | 004pA004pC004pC004pA007pC0 | |
| | | pU004pC004pU004pA004pU004 | | 04pG007pC004pU004pC004pA00 | |
| | | pU004pA004 | | 4pU004p001G004p001A004 | |
| 516 | 1588 | U004p001G004p001A004pG004 | 922 | U004p001G007p001A004pA004p | 1163 |
| | | pC004pG004pU007pG004pG007 | | U004pA007pG004pA004pU004pA | |
| | | pU007pG007pU004pA004pU004 | | 004pC004pA004pC004pC007pA0 | |
| | | pC004pU004pA004pU004pU004 | | 04pC007pG004pC004pU004pC00 | |
| | | pC004pA004 | | 4pA004p001U004p001G004 | |
| 517 | 1589 | G004p001A004p001G004pC004 | 923 | U004p001C007p001G004pA004p | 1164 |
| | | pG004pU004pG007pG004pU007 | | A004pU007pA004pG004pA004pU | |
| | | pG007pU007pA004pU004pC004 | | 004pA004pC004pA004pC007pC0 | |
| | | pU004pA004pU004pU004pC004 | | 04pA007pC004pG004pC004pU00 | |
| | | pG004pA004 | | 4pC004p001A004p001U004 | |
| 518 | 1656 | A004p001C004p001C004pU004 | 924 | U004p001A007p001U004pA004p | 1165 |
| | | pA004pC004pC007pU004pU007 | | A004pU007pC004pC004pC004pU | |
| | | pA007pC007pA004pG004pG004 | | 004pG004pU004pA004pA007pG0 | |
| | | pG004pA004pU004pU004pA004 | | 04pG007pU004pA004pG004pG00 | |
| | | pU004pA004 | | 4pU004p001A004p001C004 | |
| 519 | 1658 | C004p001U004p001A004pC004 | 925 | A004p001U007p001U004pA004p | 1166 |
| | | pC004pU004pU007pA004pC007 | | U004pA007pA004pU004pC004pC | |
| | | pA007pG007pG004pG004pA004 | | 004pC004pU004pG004pU007pA0 | |
| | | pU004pU004pA004pU004pA004 | | 04pA007pG004pG004pU004pA00 | |
| | | pA004pU004 | | 4pG004p001G004p001U004 | |
| 520 | 1664 | U004p001A004p001C004pA004 | 926 | U004p001G007p001A004pG004p | 1167 |
| | | pG004pG004pG007pA004pU007 | | U004pA007pA004pU004pU004pA | |
| | | pU007pA007pU004pA004pA004 | | 004pU004pA004pA004pU007pC0 | |
| | | pU004pU004pA004pC004pU004 | | 04pC007pC004pU004pG004pU00 | |
| | | pC004pA004 | | 4pA004p001A004p001G004 | |
| 521 | 1862 | A004p001G004p001C004pA004 | 927 | A004p001U007p001C004pU004p | 1168 |
| | | pG004pC004pC007pG004pA007 | | G004pC007pA004pA004pG004pG | |
| | | pG007pU007pC004pC004pU004 | | 004pA004pC004pU004pC007pG0 | |
| | | pU004pG004pC004pA004pG004 | | 04pG007pC004pU004pG004pC00 | |
| | | pA004pU004 | | 4pU004p001G004p001G004 | |
| 522 | 1875 | U004p001U004p001G004pC004 | 928 | U004p001A007p001C004pG004p | 1169 |
| | | pA004pG004pA007pU004pG007 | | A004pG007pU004pC004pA004pU | |
| | | pG007pG007pA004pU004pG004 | | 004pC004pC004pC004pA007pU0 | |
| | | pA004pC004pU004pC004pG004 | | 04pC007pU004pG004pC004pA00 | |
| | | pU004pA004 | | 4pA004p001G004p001G004 | |
| 523 | 1876 | U004p001G004p001C004pA004 | 929 | U004p001C007p001A004pC004p | 1170 |
| | | pG004pA004pU007pG004pG007 | | G004pA007pG004pU004pC004pA | |
| | | pG007pA007pU004pG004pA004 | | 004pU004pC004pC004pC007pA0 | |
| | | pC004pU004pC004pG004pU004 | | 04pU007pC004pU004pG004pC00 | |
| | | pG004pA004 | | 4pA004p001A004p001G004 | |
| 524 | 1878 | C004p001A004p001G004pA004 | 930 | U004p001G007p001C004pC004p | 1171 |
| | | pU004pG004pG007pG004pA007 | | A004pC007pG004pA004pG004pU | |
| | | pU007pG007pA004pC004pU004 | | 004pC004pA004pU004pC007pC0 | |
| | | pC004pG004pU004pG004pG004 | | 04pC007pA004pU004pC004pU00 | |
| | | pC004pA004 | | 4pG004p001C004p001A004 | |
| 525 | 1879 | A004p001G004p001A004pU004 | 931 | U004p001G007p001G004pC004p | 1172 |
| | | pG004pG004pG007pA004pU007 | | C004pA007pC004pG004pA004pG | |
| | | pG007pA007pC004pU004pC004 | | 004pU004pC004pA004pU007pC0 | |
| | | pG004pU004pG004pG004pC004 | | 04pC007pC004pA004pU004pC00 | |
| | | pC004pA004 | | 4pU004p001G004p001C004 | |
| 526 | 1889 | A004p001C004p001U004pC004 | 932 | A004p001C007p001G004pC004p | 1173 |
| | | pG004pU004pG007pG004pC007 | | A004pC007pA004pA004pC004pU | |
| | | pC007pC007pA004pG004pU004 | | 004pG004pG004pG004pC007pC0 | |
| | | pU004pG004pU004pG004pC004 | | 04pA007pC004pG004pA004pG00 | |
| | | pG004pU004 | | 4pU004p001C004p001A004 | |
| 527 | 1898 | C004p001C004p001A004pG004 | 933 | A004p001C007p001G004pU004p | 1174 |
| | | pU004pU004pG007pU004pG007 | | G004pG007pA004pA004pG004pA | |
| | | pc007pG007pU004pC004pU004 | | 004pC004pG004pC004pA007pC0 | |
| | | pU004pC004pC004pA004pC004 | | 04pA007pA004pC004pU004pG00 | |
| | | pG004pU004 | | 4pG004p001G004p001C004 | |
| 528 | 1901 | G004p001U004p001U004pG004 | 934 | A004p001G007p001C004pA004p | 1175 |
| | | pU004pG004pC007pG004pU007 | | C004pG007pU004pG004pG004pA | |
| | | pC007pU007pU004pC004pC004 | | 004pA004pG004pA004pC007pG0 | |
| | | pA004pC004pG004pU004pG004 | | 04pC007pA004pC004pA004pA00 | |
| | | pC004pU004 | | 4pC004p001U004p001G004 | |
| 529 | 1936 | A004p001G004p001U004pG004 | 935 | U004p001U007p001U004pU004p | 1176 |
| | | pA004pA004pA007pG004pC007 | | C004pG007pA004pG004pC004pC | |
| | | pC007pU007pG004pG004pC004 | | 004pA004pG004pG004pC007pU0 | |
| | | pU004pC004pG004pA004pA004 | | 04pU007pU004pC004pA004pC00 | |
| | | pA004pA004 | | 4pU004p001U004p001C004 | |
| 530 | 1945 | C004p001U004p001G004pG004 | 936 | U004p001C007p001U004pU004p | 1177 |
| | | pC004pU004pC007pG004pA007 | | C004pA007pG004pA004pU004pG | |
| | | pA007pA007pC004pA004pU004 | | 004pU004pU004pU004pC007pG0 | |
| | | pC004pU004pG004pA004pA004 | | 04pA007pG004pC004pC004pA00 | |
| | | pG004pA004 | | 4pG004p001G004p001C004 | |
| 531 | 1952 | G004p001A004p001A004pA004 | 937 | U004p001G007p001C004pG004p | 1178 |
| | | pC004pA004pU007pC004pU007 | | A004pA007pC004pC004pC004pU | |
| | | pG007pA007pA004pG004pG004 | | 004pU004pC004pA004pG007pA0 | |
| | | pG004pU004pU004pC004pG004 | | 04pU007pG004pU004pU004pU00 | |
| | | pC004pA004 | | 4pC004p001G004p001A004 | |
| 532 | 1953 | A004p001A004p001A004pC004 | 938 | U004p001U007p001G004pC004p | 1179 |
| | | pA004pU004pC007pU004pG007 | | G004pA007pA004pC004pC004pC | |
| | | pA007pA007pG004pG004pG004 | | 004pU004pU004pC004pA007pG0 | |
| | | pU004pU004pC004pG004pC004 | | 04pA007pU004pG004pU004pU00 | |
| | | pA004pA004 | | 4pU004p001C004p001G004 | |
| 533 | 1959 | C004p001U004p001G004pA004 | 939 | U004p001U007p001A004pU004p | 1180 |
| | | pA004pG004pG007pG004pU007 | | C004pA007pC004pU004pG004pC | |
| | | pU007pC007pG004pC004pA004 | | 004pG004pA004pA004pC007pC0 | |
| | | pG004pU004pG004pA004pU004 | | 04pC007pU004pU004pC004pA00 | |
| | | pA004pA004 | | 4pG004p001A004p001U004 | |
| 534 | 1961 | G004p001A004p001A004pG004 | 940 | U004p001U007p001U004pU004p | 1181 |
| | | pG004pG004pU007pU004pC007 | | A004pU007pC004pA004pC004pU | |
| | | pG007pC007pA004pG004pU004 | | 004pG004pC004pG004pA007pA0 | |
| | | pG004pA004pU004pA004pA004 | | 04pC007pC004pC004pU004pU00 | |
| | | pA004pA004 | | 4pC004p001A004p001G004 | |
| 535 | 1985 | G004p001C004p001A004pU004 | 941 | U004p001C007p001U004pG004p | 1182 |
| | | pU004pU004pG007pA004pC007 | | C004pU007pA004pG004pU004pG | |
| | | pA007pG007pC004pA004pC004 | | 004pC004pU004pG004pU007pC0 | |
| | | pU004pA004pG004pC004pA004 | | 04pA007pA004pA004pU004pG00 | |
| | | pG004pA004 | | 4pC004p001C004p001U004 | |
| 536 | 1989 | U004p001U004p001G004pA004 | 942 | U004p001A007p001A004pA004p | 1183 |
| | | pC004pA004pG007pC004pA007 | | U004pC007pU004pG004pC004pU | |
| | | pC007pU007pA004pG004pC004 | | 004pA004pG004pU004pG007pC0 | |
| | | pA004pG004pA004pU004pU004 | | 04pU007pG004pU004pC004pA00 | |
| | | pU004pA004 | | 4pA004p001A004p001U004 | |
| 537 | 1995 | G004p001C004p001A004pC004 | 943 | U004p001A007p001C004pG004p | 1184 |
| | | pU004pA004pG007pC004pA007 | | U004pG007pC004pA004pA004pA | |
| | | pG007pA007pU004pU004pU004 | | 004pU004pC004pU004pG007pC0 | |
| | | pG004pC004pA004pC004pG004 | | 04pU007pA004pG004pU004pG00 | |
| | | pU004pA004 | | 4pC004p001U004p001G004 | |
| 538 | 1996 | C004p001A004p001C004pU004 | 944 | A004p001G007p001A004pC004p | 1185 |
| | | pA004pG004pC007pA004pG007 | | G004pU007pG004pC004pA004pA | |
| | | pA007pU007pU004pU004pG004 | | 004pA004pU004pC004pU007pG0 | |
| | | pC004pA004pC004pG004pU004 | | 04pC007pU004pA004pG004pU00 | |
| | | pC004pU004 | | 4pG004p001C004p001U004 | |
| 539 | 1998 | C004p001U004p001A004pG004 | 945 | U004p001U007p001A004pG004p | 1186 |
| | | pC004pA004pG007pA004pU007 | | A004pC007pG004pU004pG004pC | |
| | | pU007pU007pG004pC004pA004 | | 004pA004pA004pA004pU007pC0 | |
| | | pC004pG004pU004pC004pU004 | | 04pU007pG004pC004pU004pA00 | |
| | | pA004pA004 | | 4pG004p001U004p001G004 | |
| 540 | 1999 | U004p001A004p001G004pC004 | 946 | U004p001G007p001U004pA004p | 1187 |
| | | pA004pG004pA007pU004pU007 | | G004pA007pC004pG004pU004pG | |
| | | pU007pG007pC004pA004pC004 | | 004pC004pA004pA004pA007pU0 | |
| | | pG004pU004pC004pU004pA004 | | 04pC007pU004pG004pC004pU00 | |
| | | pC004pA004 | | 4pA004p001G004p001U004 | |
| 541 | 2000 | A004p001G004p001C004pA004 | 947 | U004p001U007p001G004pU004p | 1188 |
| | | pG004pA004pU007pU004pU007 | | A004pG007pA004pC004pG004pU | |
| | | pG007pC007pA004pC004pG004 | | 004pG004pC004pA004pA007pA0 | |
| | | pU004pC004pU004pA004pC004 | | 04pU007pC004pU004pG004pC00 | |
| | | pA004pA004 | | 4pU004p001A004p001G004 | |
| 542 | 2004 | G004p001A004p001U004pU004 | 948 | U004p001U007p001U004pU004p | 1189 |
| | | pU004pG004pC007pA004pC007 | | C004pU007pG004pU004pA004pG | |
| | | pG007pU007pC004pU004pA004 | | 004pA004pC004pG004pU007pG0 | |
| | | pC004pA004pG004pA004pA004 | | 04pC007pA004pA004pA004pU00 | |
| | | pA004pA004 | | 4pC004p001U004p001G004 | |
| 543 | 2006 | U004p001U004p001U004pG004 | 949 | A004p001A007p001G004pU004p | 1190 |
| | | pC004pA004pC007pG004pU007 | | U004pU007pC004pU004pG004pU | |
| | | pC007pU007pA004pC004pA004 | | 004pA004pG004pA004pC007pG0 | |
| | | pG004pA004pA004pA004pC004 | | 04pU007pG004pC004pA004pA00 | |
| | | pU004pU004 | | 4pA004p001U004p001C004 | |
| 544 | 2007 | U004p001U004p001G004pC004 | 950 | U004p001A007p001A004pG004p | 1191 |
| | | pA004pC004pG007pU004pC007 | | U004pU007pU004pC004pU004pG | |
| | | pU007pA007pC004pA004pG004 | | 004pU004pA004pG004pA007pC0 | |
| | | pA004pA004pA004pC004pU004 | | 04pG007pU004pG004pC004pA00 | |
| | | pU004pA004 | | 4pA004p001A004p001U004 | |
| 545 | 2040 | C004p001U004p001G004pG004 | 951 | U004p001G007p001A004pU004p | 1192 |
| | | pA004pC004pG007pC004pA007 | | A004pU007pA004pA004pA004pG | |
| | | pA007pC007pC004pU004pU004 | | 004pG004pU004pU004pG007pC0 | |
| | | pU004pA004pU004pA004pU004 | | 04pG007pU004pC004pC004pA00 | |
| | | pC004pA004 | | 4pG004p001C004p001U004 | |
| 546 | 2042 | G004p001G004p001A004pC004 | 952 | A004p001C007p001G004pG004p | 1193 |
| | | pG004pC004pA007pA004pC007 | | A004pU007pA004pU004pA004pA | |
| | | pC007pU007pU004pU004pA004 | | 004pA004pG004pG004pU007pU0 | |
| | | pU004pA004pU004pC004pC004 | | 04pG007pC004pG004pU004pC00 | |
| | | pG004pU004 | | 4pC004p001A004p001G004 | |
| 547 | 2043 | G004p001A004p001C004pG004 | 953 | A004p001A007p001C004pG004p | 1194 |
| | | pC004pA004pA007pC004pC007 | | G004pA007pU004pA004pU004pA | |
| | | pU007pU007pU004pA004pU004 | | 004pA004pA004pG004pG007pU0 | |
| | | pA004pU004pC004pC004pG004 | | 04pU007pG004pC004pG004pU00 | |
| | | pU004pU004 | | 4pC004p001C004p001A004 | |
| 548 | 2170 | C004p001G004p001U004pU004 | 954 | U004p001U007p001A004pC004p | 1195 |
| | | pA004pG004pU007pG004pG007 | | A004pA007pU004pA004pG004pU | |
| | | pU007pA007pA004pC004pU004 | | 004pU004pA004pC004pC007pA0 | |
| | | pA004pU004pU004pG004pU004 | | 04pC007pU004pA004pA004pC00 | |
| | | pA004pA004 | | 4pG004p001G004p001C004 | |
| 549 | 2172 | U004p001U004p001A004pG004 | 953 | U004p001A007p001G004pU004p | 1196 |
| | | pU004pG004pG007pU004pA007 | | A004pC007pA004pA004pU004pA | |
| | | pA007pC007pU004pA004pU004 | | 004pG004pU004pU004pA007pC0 | |
| | | pU004pG004pU004pA004pC004 | | 04pC007pA004pC004pU004pA00 | |
| | | pU004pA004 | | 4pA004p001C004p001G004 | |
| 550 | 2179 | U004p001A004p001A004pC004 | 956 | U004p001U007p001C004pU004p | 1197 |
| | | pU004pA004pU007pU004pG007 | | U004pG007pU004pC004pA004pG | |
| | | pU007pA007pC004pU004pG004 | | 004pU004pA004pC004pA007pA0 | |
| | | pA004pC004pA004pA004pG004 | | 04pU007pA004pG004pU004pU00 | |
| | | pA004pA004 | | 4pA004p001C004p001C004 | |
| 551 | 2183 | U004p001A004p001U004pU004 | 957 | A004p001G007p001G004pU004p | 1198 |
| | | pG004pU004pA007pC004pU007 | | U004pU007pC004pU004pU004pG | |
| | | pG007pA007pC004pA004pA004 | | 004pU004pC004pA004pG007pU0 | |
| | | pG004pA004pA004pA004pC004 | | 04pA007pC004pA004pA004pU00 | |
| | | pC004pU004 | | 4pA004p001G004p001U004 | |
| 552 | 2193 | A004p001C004p001A004pA004 | 958 | U004p001U007p001A004pU004p | 1199 |
| | | pG004pA004pA007pA004pC007 | | A004pG007pC004pA004pG004pC | |
| | | pC007pU007pG004pC004pU004 | | 004pA004pG004pG004pU007pU0 | |
| | | pG004pC004pU004pA004pU004 | | 04pU007pC004pU004pU004pG00 | |
| | | pA004pA004 | | 4pU004p001C004p001A004 | |
| 553 | 2264 | G004p001C004p001C004pA004 | 959 | U004p001A007p001C004pU004p | 1200 |
| | | pA004pG004pG007pU004pU007 | | U004pC007pU004pC004pU004pG | |
| | | pG007pU007pC004pA004pG004 | | 004pA004pC004pA004pA007pC0 | |
| | | pA004pG004pA004pA004pG004 | | 04pC007pU004pU004pG004pG00 | |
| | | pU004pA004 | | 4pC004p001U004p001G004 | |
| 554 | 2266 | C004p001A004p001A004pG004 | 960 | A004p001A007p001U004pA004p | 1201 |
| | | pG004pU004pU007pG004pU007 | | C004pU007pU004pC004pU004pC | |
| | | pC007pA007pG004pA004pG004 | | 004pU004pG004pA004pC007pA0 | |
| | | pA004pA004pG004pU004pA004 | | 04pA007pC004pC004pU004pU00 | |
| | | pU004pU004 | | 4pG004p001G004p001C004 | |
| 555 | 2269 | G004p001G004p001U004pU004 | 961 | U004p001U007p001U004pA004p | 1202 |
| | | pG004pU004pC007pA004pG007 | | A004pU007pA004pC004pU004pU | |
| | | pA007pG007pA004pA004pG004 | | 004pC004pU004pC004pU007pG0 | |
| | | pU004pA004pU004pU004pA004 | | 04pA007pC004pA004pA004pC00 | |
| | | pA004pA004 | | 4pC004p001U004p001U004 | |
| 556 | 2274 | U004p001C004p001A004pG004 | 962 | U004p001A007p001G004pU004p | 1203 |
| | | pA004pG004pA007pA004pG007 | | C004pU007pU004pU004pA004pA | |
| | | pU007pA007pU004pU004pA004 | | 004pU004pA004pC004pU007pU0 | |
| | | pA004pA004pG004pA004pC004 | | 04pC007pU004pC004pU004pG00 | |
| | | pU004pA004 | | 4pA004p001C004p001A004 | |
| 557 | 2276 | A004p001G004p001A004pG004 | 963 | U004p001G007p001U004pA004p | 1204 |
| | | pA004pA004pG007pU004pA007 | | G004pU007pC004pU004pU004pU | |
| | | pU007pU007pA004pA004pA004 | | 004pA004pA004pU004pA007pC0 | |
| | | pG004pA004pC004pU004pA004 | | 04pU007pU004pC004pU004pC00 | |
| | | pC004pA004 | | 4pU004p001G004p001A004 | |
| 558 | 2277 | G004p001A004p001G004pA004 | 964 | U004p001G007p001G004pU004p | 1205 |
| | | pA004pG004pU007pA004pU007 | | A004pG007pU004pC004pU004pU | |
| | | pU007pA007pA004pA004pG004 | | 004pU004pA004pA004pU007pA0 | |
| | | pA004pC004pU004pA004pC004 | | 04pC007pU004pU004pC004pU00 | |
| | | pC004pA004 | | 4pC004p001U004p001G004 | |
| 559 | 2278 | A004p001G004p001A004pA004 | 965 | U004p001U007p001G004pG004p | 1206 |
| | | pG004pU004pA007pU004pU007 | | U004pA007pG004pU004pC004pU | |
| | | pA007pA007pA004pG004pA004 | | 004pU004pU004pA004pA007pU0 | |
| | | pC004pU004pA004pC004pC004 | | 04pA007pC004pU004pU004pC00 | |
| | | pA004pA004 | | 4pU004p001C004p001U004 | |
| 560 | 2300 | G004p001A004p001G004pG004 | 966 | U004p001U007p001U004pG004p | 1207 |
| | | pC004pU004pA007pU004pG007 | | A004pC007pC004pU004pC004pA | |
| | | pA007pU007pU004pG004pA004 | | 004pA004pU004pC004pA007pU0 | |
| | | pG004pG004pU004pC004pA004 | | 04pA007pG004pC004pC004pU00 | |
| | | pA004pA004 | | 4pC004p001U004p001G004 | |
| 561 | 2301 | A004p001G004p001G004pC004 | 967 | U004p001G007p001U004pU004p | 1208 |
| | | pU004pA004pU007pG004pA007 | | G004pA007pC004pC004pU004pC | |
| | | pU007pU007pG004pA004pG004 | | 004pA004pA004pU004pC007pA0 | |
| | | pG004pU004pC004pA004pA004 | | 04pU007pA004pG004pC004pC00 | |
| | | pC004pA004 | | 4pU004p001C004p001U004 | |
| 562 | 2305 | U004p001A004p001U004pG004 | 968 | U004p001U007p001A004pA004p | 1209 |
| | | pA004pU004pU007pG004pA007 | | U004pG007pU004pU004pG004pA | |
| | | pG007pG007pU004pC004pA004 | | 004pC004pC004pU004pC007pA0 | |
| | | pA004pC004pA004pU004pU004 | | 04pA007pU004pC004pA004pU00 | |
| | | pA004pA004 | | 4pA004p001G004p001C004 | |
| 563 | 2306 | A004p001U004p001G004pA004 | 969 | U004p001U007p001U004pA004p | 1210 |
| | | pU004pU004pG007pA004pG007 | | A004pU007pG004pU004pU004pG | |
| | | pG007pU007pC004pA004pA004 | | 004pA004pC004pC004pU007pC0 | |
| | | pC004pA004pU004pU004pA004 | | 04pA007pA004pU004pC004pA00 | |
| | | pA004pA004 | | 4pU004p001A004p001G004 | |
| 564 | 2320 | C004p001A004p001U004pU004 | 970 | U004p001C007p001C004pA004p | 1211 |
| | | pA004pA004pC007pA004pA007 | | C004pU007pA004pA004pA004pU | |
| | | pG007pA007pA004pU004pU004 | | 004pU004pC004pU004pU007pG0 | |
| | | pU004pA004pG004pU004pG004 | | 04pU007pU004pA004pA004pU00 | |
| | | pG004pA004 | | 4pG004p001U004p001U004 | |
| 565 | 2323 | U004p001A004p001A004pC004 | 971 | U004p001A007p001G004pC004p | 1212 |
| | | pA004pA004pG007pA004pA007 | | C004pC007pA004pC004pU004pA | |
| | | pU007pU007pU004pA004pG004 | | 004pA004pA004pU004pU007pC0 | |
| | | pU004pG004pG004pG004pC004 | | 04pU007pU004pG004pU004pU00 | |
| | | pU004pA004 | | 4pA004p001A004p001U004 | |
| 566 | 2328 | A004p001G004p001A004pA004 | 972 | U004p001G007p001G004pC004p | 1213 |
| | | pU004pU004pU007pA004pG007 | | A004pG007pA004pG004pC004pC | |
| | | pU007pG007pG004pG004pC004 | | 004pC004pA004pC004pU007pA0 | |
| | | pU004pC004pU004pG004pC004 | | 04pA007pA004pU004pU004pC00 | |
| | | pC004pA004 | | 4pU004p001U004p001G004 | |
| 567 | 2344 | U004p001G004p001C004pC004 | 973 | U004p001C007p001U004pA004p | 1214 |
| | | pA004pU004pG007pG004pC007 | | U004pG007pC004pU004pC004pC | |
| | | pU007pG007pG004pG004pA004 | | 004pC004pA004pG004pC007pC0 | |
| | | pG004pC004pA004pU004pA004 | | 04pA007pU004pG004pG004pC00 | |
| | | pG004pA004 | | 4pA004p001G004p001A004 | |
| 568 | 2345 | G004p001C004p001C004pA004 | 974 | U004p001C007p001C004pU004p | 1215 |
| | | pU004pG004pG007pC004pU007 | | A004pU007pG004pC004pU004pC | |
| | | pG007pG007pG004pA004pG004 | | 004pC004pC004pA004pG007pC0 | |
| | | pC004pA004pU004pA004pG004 | | 04pC007pA004pU004pG004pG00 | |
| | | pG004pA004 | | 4pC004p001A004p001G004 | |
| 569 | 2357 | A004p001G004p001C004pA004 | 975 | U004p001G007p001C004pG004p | 1216 |
| | | pU004pA004pG007pG004pA007 | | U004pU007pG004pU004pA004pG | |
| | | pG007pG007pC004pU004pA004 | | 004pC004pC004pU004pC007pC0 | |
| | | pC004pA004pA004pC004pG004 | | 04pU007pA004pU004pG004pC00 | |
| | | pC004pA004 | | 4pU004p001C004p001C004 | |
| 570 | 2358 | G004p001C004p001A004pU004 | 976 | U004p001G007p001G004pC004p | 1217 |
| | | pA004pG004pG007pA004pG007 | | G004pU007pU004pG004pU004pA | |
| | | pG007pC007pU004pA004pC004 | | 004pG004pC004pC004pU007pC0 | |
| | | pA004pA004pC004pG004pC004 | | 04pC007pU004pA004pU004pG00 | |
| | | pC004pA004 | | 4pC004p001U004p001C004 | |
| 571 | 2362 | A004p001G004p001G004pA004 | 977 | U004p001C007p001A004pU004p | 1218 |
| | | pG004pG004pC007pU004pA007 | | G004pG007pG004pC004pG004pU | |
| | | pC007pA007pA004pC004pG004 | | 004pU004pG004pU004pA007pG0 | |
| | | pc004pC004pC004pA004pU004 | | 04pC007pC004pU004pC004pC00 | |
| | | pG004pA004 | | 4pU004p001A004p001U004 | |
| 572 | 2368 | C004p001U004p001A004pC004 | 978 | U004p001U007p001U004pG004p | 1219 |
| | | pA004pA004pC007pG004pC007 | | C004pU007pG004pC004pA004pU | |
| | | pC007pC007pA004pU004pG004 | | 004pG004pG004pG004pC007pG0 | |
| | | pC004pA004pG004pC004pA004 | | 04pU007pU004pG004pU004pA00 | |
| | | pA004pA004 | | 4pG004p001C004p001C004 | |
| 573 | 2370 | A004p001C004p001A004pA004 | 979 | U004p001G007p001U004pU004p | 1220 |
| | | pC004pG004pC007pC004pC007 | | U004pG007pC004pU004pG004pC | |
| | | pA007pU007pG004pC004pA004 | | 004pA004pU004pG004pG007pG0 | |
| | | pG004pC004pA004pA004pA004 | | 04pC007pG004pU004pU004pG00 | |
| | | pC004pA004 | | 4pU004p001A004p001G004 | |
| 574 | 2376 | C004p001C004p001C004pA004 | 980 | U004p001G007p001A004pC004p | 1221 |
| | | pU004pG004pC007pA004pG007 | | A004pA007pU004pG004pU004pU | |
| | | pC007pA007pA004pA004pC004 | | 004pU004pG004pC004pU007pG0 | |
| | | pA004pU004pU004pG004pU004 | | 04pC007pA004pU004pG004pG00 | |
| | | pC004pA004 | | 4pG004p001C004p001G004 | |
| 575 | 2377 | C004p001C004p001A004pU004 | 981 | U004p001U007p001G004pA004p | 1222 |
| | | pG004pC004pA007pG004pC007 | | C004pA007pA004pU004pG004pU | |
| | | pA007pA007pA004pC004pA004 | | 004pU004pU004pG004pC007pU0 | |
| | | pU004pU004pG004pU004pC004 | | 04pG007pC004pA004pU004pG00 | |
| | | pA004pA004 | | 4pG004p001G004p001C004 | |
| 576 | 2399 | G004p001C004p001C004pA004 | 982 | U004p001C007p001C004pA004p | 1223 |
| | | pU004pC004pU007pA004pC007 | | C004pA007pG004pG004pC004pA | |
| | | pA007pU007pU004pG004pC004 | | 004pA004pU004pG004pU007pA0 | |
| | | pC004pU004pG004pU004pG004 | | 04pG007pA004pU004pG004pG00 | |
| | | pG004pA004 | | 4pC004p001G004p001G004 | |
| 577 | 2424 | A004p001U004p001G004pC004 | 983 | U004p001A007p001C004pC004p | 1224 |
| | | pA004pG004pC007pA004pC007 | | A004pA007pC004pA004pU004pU | |
| | | pA007pG007pA004pA004pU004 | | 004pC004pU004pG004pU007pG0 | |
| | | pG004pU004pU004pG004pG004 | | 04pC007pU004pG004pC004pA00 | |
| | | pU004pA004 | | 4pU004p001C004p001C004 | |
| 578 | 2426 | G004p001C004p001A004pG004 | 984 | A004p001C007p001U004pA004p | 1225 |
| | | pC004pA004pC007pA004pG007 | | C004pC007pA004pA004pC004pA | |
| | | pA007pA007pU004pG004pU004 | | 004pU004pU004pC004pU007pG0 | |
| | | pU004pG004pG004pU004pA004 | | 04pU007pG004pC004pU004pG00 | |
| | | pG004pU004 | | 4pC004p001A004p001U004 | |
| 579 | 2429 | G004p001C004p001A004pC004 | 985 | U004p001G007p001A004pA004p | 1226 |
| | | pA004pG004pA007pA004pU007 | | C004pU007pA004pC004pC004pA | |
| | | pG007pU007pU004pG004pG004 | | 004pA004pC004pA004pU007pU0 | |
| | | pU004pA004pG004pU004pU004 | | 04pC007pU004pG004pU004pG00 | |
| | | pC004pA004 | | 4pC004p001U004p001G004 | |
| 580 | 2479 | U004p001C004p001C004pC004 | 986 | U004p001A007p001U004pA004p | 1227 |
| | | pA004pC004pA007pA004pA007 | | A004pA007pU004pC004pU004pU | |
| | | pU007pG007pA004pA004pG004 | | 004pC004pA004pU004pU007pU0 | |
| | | pA004pU004pU004pU004pA004 | | 04pG007pU004pG004pG004pG00 | |
| | | pU004pA004 | | 4pA004p001C004p001C004 | |
| 581 | 2482 | C004p001A004p001C004pA004 | 987 | A004p001U007p001A004pU004p | 1228 |
| | | pA004pA004pU007pG004pA007 | | A004pU007pA004pA004pA004pU | |
| | | pA007pG007pA004pU004pU004 | | 004pC004pU004pU004pC007pA0 | |
| | | pU004pA004pU004pA004pU004 | | 04pU007pU004pU004pG004pU00 | |
| | | pA004pU004 | | 4pG004p001G004p001G004 | |
| 582 | 2484 | C004p001A004p001A004pA004 | 988 | U004p001G007p001A004pU004p | 1229 |
| | | pU004pG004pA007pA004pG007 | | A004pU007pA004pU004pA004pA | |
| | | pA007pU007pU004pU004pA004 | | 004pA004pU004pC004pU007pU0 | |
| | | pU004pA004pU004pA004pU004 | | 04pC007pA004pU004pU004pU00 | |
| | | pC004pA004 | | 4pG004p001U004p001G004 | |
| 583 | 2502 | U004p001C004p001A004pG004 | 989 | U004p001A007p001G004pA004p | 1230 |
| | | pC004pU004pG007pC004pA007 | | U004pG007pG004pC004pA004pU | |
| | | pC007pC007pA004pU004pG004 | | 004pG004pG004pU004pG007pC0 | |
| | | pC004pC004pA004pU004pC004 | | 04pA007pG004pC004pU004pG00 | |
| | | pU004pA004 | | 4pA004p001U004p001A004 | |
| 584 | 2575 | U004p001U004p001U004pG004 | 990 | U004p001G007p001A004pA004p | 1231 |
| | | pC004pA004pG007pA004pU007 | | C004pA007pC004pC004pU004pA | |
| | | pG007pC007pU004pA004pG004 | | 004pG004pC004pA004pU007pC0 | |
| | | pG004pU004pG004pU004pU004 | | 04pU007pG004pC004pA004pA00 | |
| | | pC004pA004 | | 4pA004p001C004p001A004 | |
| 585 | 2576 | U004p001U004p001G004pC004 | 991 | U004p001U007p001G004pA004p | 1232 |
| | | pA004pG004pA007pU004pG007 | | A004pC007pA004pC004pC004pU | |
| | | pC007pU007pA004pG004pG004 | | 004pA004pG004pC004pA007pU0 | |
| | | pU004pG004pU004pU004pC004 | | 04pC007pU004pG004pC004pA00 | |
| | | pA004pA004 | | 4pA004p001A004p001C004 | |
| 586 | 2579 | C004p001A004p001G004pA004 | 992 | U004p001C007p001C004pU004p | 1233 |
| | | pU004pG004pC007pU004pA007 | | U004pG007pA004pA004pC004pA | |
| | | pG007pG007pU004pG004pU004 | | 004pC004pC004pU004pA007pG0 | |
| | | pU004pC004pA004pA004pG004 | | 04pC007pA004pU004pC004pU00 | |
| | | pG004pA004 | | 4pG004p001C004p001A004 | |
| 587 | 2592 | U004p001U004p001C004pA004 | 993 | U004p001A007p001U004pC004p | 1234 |
| | | pA004pG004pG007pA004pG007 | | U004pU007pU004pG004pC004pA | |
| | | pC007pA007pU004pG004pC004 | | 004pU004pG004pC004pU007pC0 | |
| | | pA004pA004pA004pG004pA004 | | 04pC007pU004pU004pG004pA00 | |
| | | pU004pA004 | | 4pA004p001C004p001A004 | |
| 588 | 2606 | A004p001A004p001A004pG004 | 994 | A004p001U007p001U004pU004p | 1235 |
| | | pA004pU004pA007pA004pU007 | | U004pC007pC004pC004pC004pA | |
| | | pC007pC007pU004pG004pG004 | | 004pG004pG004pA004pU007pU0 | |
| | | pG004pG004pA004pA004pA004 | | 04pA007pU004pC004pU004pU00 | |
| | | pA004pU004 | | 4pU004p001G004p001C004 | |
| 589 | 2620 | G004p001G004p001A004pA004 | 995 | U004p001C007p001A004pA004p | 1236 |
| | | pA004pA004pU007pG004pC007 | | G004pC007pU004pG004pC004pC | |
| | | pC007pC007pG004pG004pC004 | | 004pG004pG004pG004pC007pA0 | |
| | | pA004pG004pC004pU004pU004 | | 04pU007pU004pU004pU004pC00 | |
| | | pG004pA004 | | 4pC004p001C004p001C004 | |
| 590 | 2627 | G004p001C004p001C004pC004 | 996 | A004p001A007p001U004pU004p | 1237 |
| | | pG004pG004pC007pA004pG007 | | C004pG007pG004pG004pC004pA | |
| | | pc007pU007pU004pG004pC004 | | 004pA004pG004pC004pU007pG0 | |
| | | pC004pC004pG004pA004pA004 | | 04pC007pC004pG004pG004pG00 | |
| | | pU004pU004 | | 4pC004p001A004p001U004 | |
| 591 | 2633 | C004p001A004p001G004pC004 | 997 | A004p001C007p001A004pC004p | 1238 |
| | | pU004pU004pG007pC004pC007 | | A004pC007pA004pA004pU004pU | |
| | | pC007pG007pA004pA004pU004 | | 004pC004pG004pG004pG007pC0 | |
| | | pU004pG004pU004pG004pU004 | | 04pA007pA004pG004pC004pU00 | |
| | | pG004pU004 | | 4pG004p001C004p001C004 | |
| 592 | 2658 | C004p001C004p001G004pU004 | 998 | A004p001C007p001A004pA004p | 1239 |
| | | pA004pA004pU007pG004pG007 | | U004pU007pC004pC004pC004pC | |
| | | pC007pU007pG004pG004pG004 | | 004pA004pG004pC004pC007pA0 | |
| | | pG004pA004pA004pU004pU004 | | 04pU007pU004pA004pC004pG00 | |
| | | pG004pU004 | | 4pG004p001U004p001C004 | |
| 593 | 2677 | G004p001U004p001C004pA004 | 999 | U004p001C007p001C004pA004p | 1240 |
| | | pc004pU004pU007pA004pU007 | | A004pU007pG004pC004pU004pG | |
| | | pG007pG007pC004pA004pG004 | | 004pC004pC004pA004pU007pA0 | |
| | | pC004pA004pU004pU004pG004 | | 04pA007pG004pU004pG004pA00 | |
| | | pG004pA004 | | 4pC004p001A004p001A004 | |
| 594 | 2721 | A004p001C004p001A004pU004 | 1000 | U004p001C007p001G004pA004p | 1241 |
| | | pG004pA004pU007pU004pC007 | | C004pC007pU004pG004pU004pU | |
| | | pA007pC007pA004pA004pC004 | | 004pG004pU004pG004pA007pA0 | |
| | | pA004pG004pG004pU004pC004 | | 04pU007pC004pA004pU004pG00 | |
| | | pG004pA004 | | 4pU004p001G004p001A004 | |
| 595 | 2722 | C004p001A004p001U004pG004 | 1001 | U004p001U007p001C004pG004p | 1242 |
| | | pA004pU004pU007pC004pA007 | | A004pC007pC004pU004pG004pU | |
| | | pC007pA007pA004pC004pA004 | | 004pU004pG004pU004pG007pA0 | |
| | | pG004pG004pU004pC004pG004 | | 04pA007pU004pC004pA004pU00 | |
| | | pA004pA004 | | 4pG004p001U004p001G004 | |
| 596 | 2723 | A004p001U004p001G004pA004 | 1002 | U004p001U007p001U004pC004p | 1243 |
| | | pU004pU004pC007pA004pC007 | | G004pA007pC004pC004pU004pG | |
| | | pA007pA007pC004pA004pG004 | | 004pU004pU004pG004pU007pG0 | |
| | | pG004pU004pC004pG004pA004 | | 04pA007pA004pU004pC004pA00 | |
| | | pA004pA004 | | 4pU004p001G004p001U004 | |
| 597 | 2724 | U004p001G004p001A004pU004 | 1003 | U004p001C007p001U004pU004p | 1244 |
| | | pU004pC004pA007pC004pA007 | | C004pG007pA004pC004pC004pU | |
| | | pA007pC007pA004pG004pG004 | | 004pG004pU004pU004pG007pU0 | |
| | | pU004pC004pG004pA004pA004 | | 04pG007pA004pA004pU004pC00 | |
| | | pG004pA004 | | 4pA004p001U004p001G004 | |
| 598 | 2725 | G004p001A004p001U004pU004 | 1004 | A004p001U007p001C004pU004p | 1245 |
| | | pC004pA004pC007pA004pA007 | | U004pC007pG004pA004pC004pC | |
| | | pC007pA007pG004pG004pU004 | | 004pU004pG004pU004pU007pG0 | |
| | | pC004pG004pA004pA004pG004 | | 04pU007pG004pA004pA004pU00 | |
| | | pA004pU004 | | 4pC004p001A004p001U004 | |
| 599 | 2728 | U004p001C004p001A004pC004 | 1005 | U004p001U007p001G004pA004p | 1246 |
| | | pA004pA004pC007pA004pG007 | | U004pC007pU004pU004pC004pG | |
| | | pG007pU007pC004pG004pA004 | | 004pA004pC004pC004pU007pG0 | |
| | | pA004pG004pA004pU004pC004 | | 04pU007pU004pG004pU004pG00 | |
| | | pA004pA004 | | 4pA004p001A004p001U004 | |
| 600 | 2731 | C004p001A004p001A004pC004 | 1006 | A004p001A007p001A004pU004p | 1247 |
| | | pA004pG004pG007pU004pC007 | | U004pG007pA004pU004pC004pU | |
| | | pG007pA007pA004pG004pA004 | | 004pU004pC004pG004pA007pC0 | |
| | | pU004pC004pA004pA004pU004 | | 04pC007pU004pG004pU004pU00 | |
| | | pU004pU004 | | 4pG004p001U004p001G004 | |
| 601 | 2732 | A004p001A004p001C004pA004 | 1007 | U004p001A007p001A004pA004p | 1248 |
| | | pG004pG004pU007pC004pG007 | | U004pU007pG004pA004pU004pC | |
| | | pA007pA007pG004pA004pU004 | | 004pU004pU004pC004pG007pA0 | |
| | | pC004pA004pA004pU004pU004 | | 04pC007pC004pU004pG004pU00 | |
| | | pU004pA004 | | 4pU004p001G004p001U004 | |
| 602 | 2734 | C004p001A004p001G004pG004 | 1008 | U004p001G007p001U004pA004p | 1249 |
| | | pU004pC004pG007pA004pA007 | | A004pA007pU004pU004pG004pA | |
| | | pG007pA007pU004pC004pA004 | | 004pU004pC004pU004pU007pC0 | |
| | | pA004pU004pU004pU004pA004 | | 04pG007pA004pC004pC004pU00 | |
| | | pC004pA004 | | 4pG004p001U004p001U004 | |
| 603 | 2735 | A004p001G004p001G004pU004 | 1009 | U004p001U007p001G004pU004p | 1250 |
| | | pC004pG004pA007pA004pG007 | | A004pA007pA004pU004pU004pG | |
| | | pA007pU007pC004pA004pA004 | | 004pA004pU004pC004pU007pU0 | |
| | | pU004pU004pU004pA004pC004 | | 04pC007pG004pA004pC004pC00 | |
| | | pA004pA004 | | 4pU004p001G004p001U004 | |
| 604 | 2736 | G004p001G004p001U004pC004 | 1010 | U004p001U007p001U004pG004p | 1251 |
| | | pG004pA004pA007pG004pA007 | | U004pA007pA004pA004pU004pU | |
| | | pU007pC007pA004pA004pU004 | | 004pG004pA004pU004pC007pU0 | |
| | | pU004pU004pA004pC004pA004 | | 04pU007pC004pG004pA004pC00 | |
| | | pA004pA004 | | 4pC004p001U004p001G004 | |
| 605 | 2741 | A004p001A004p001G004pA004 | 1011 | U004p001A007p001G004pG004p | 1252 |
| | | pU004pC004pA007pA004pU007 | | U004pC007pU004pU004pG004pU | |
| | | pU007pU007pA004pC004pA004 | | 004pA004pA004pA004pU007pU0 | |
| | | pA004pG004pA004pC004pC004 | | 04pG007pA004pU004pC004pU00 | |
| | | pU004pA004 | | 4pU004p001C004p001G004 | |
| 606 | 2742 | A004p001G004p001A004pU004 | 1012 | U004p001G007p001A004pG004p | 1253 |
| | | pC004pA004pA007pU004pU007 | | G004pU007pC004pU004pU004pG | |
| | | pU007pA007pC004pA004pA004 | | 004pU004pA004pA004pA007pU0 | |
| | | pG004pA004pC004pC004pU004 | | 04pU007pG004pA004pU004pC00 | |
| | | pC004pA004 | | 4pU004p001U004p001C004 | |
| 607 | 2833 | U004p001A004p001A004pA004 | 1013 | A004p001G007p001A004pU004p | 1254 |
| | | pG004pG004pA007pC004pU007 | | U004pU007pU004pA004pU004pG | |
| | | pA007pA007pC004pA004pU004 | | 004pU004pU004pA004pG007pU0 | |
| | | pA004pA004pA004pA004pU004 | | 04pC007pC004pU004pU004pU00 | |
| | | pC004pU004 | | 4pA004p001G004p001A004 | |
| 608 | 2834 | A004p001A004p001A004pG004 | 1014 | U004p001A007p001G004pA004p | 1255 |
| | | pG004pA004pC007pU004pA007 | | U004pU007pU004pU004pA004pU | |
| | | pA007pC007pA004pU004pA004 | | 004pG004pU004pU004pA007pG0 | |
| | | pA004pA004pA004pU004pC004 | | 04pU007pC004pC004pU004pU00 | |
| | | pU004pA004 | | 4pU004p001A004p001G004 | |
| 609 | 2941 | A004p001G004p001A004pG004 | 1015 | U004p001G007p001G004pA004p | 1256 |
| | | pG004pU004pC007pU004pC007 | | A004pA007pG004pA004pA004pC | |
| | | pA007pG007pG004pU004pU004 | | 004pC004pU004pG004pA007pG0 | |
| | | pC004pU004pU004pU004pC004 | | 04pA007pC004pC004pU004pC00 | |
| | | pC004pA004 | | 4pU004p001C004p001U004 | |
| 610 | 2994 | G004p001C004p001U004pU004 | 1016 | A004p001A007p001A004pG004p | 1257 |
| | | pU004pA004pC007pA004pU007 | | A004pG007pC004pA004pC004pA | |
| | | pG007pC007pU004pG004pU004 | | 004pG004pC004pA004pU007pG0 | |
| | | pG004pC004pU004pC004pU004 | | 04pU007pA004pA004pA004pG00 | |
| | | pU004pU004 | | 4pC004p001A004p001C004 | |
| 611 | 3058 | U004p001G004p001G004pU004 | 1017 | U004p001A007p001G004pG004p | 1258 |
| | | pA004pA004pU007pC004pU007 | | U004pG007pA004pG004pC004pU | |
| | | pA007pC007pA004pG004pC004 | | 004pG004pU004pA004pG007pA0 | |
| | | pU004pC004pA004pC004pC004 | | 04pU007pU004pA004pC004pC00 | |
| | | pU004pA004 | | 4pA004p001U004p001C004 | |
| 612 | 3071 | C004p001U004p001C004pA004 | 1018 | A004p001U007p001A004pU004p | 1259 |
| | | pC004pC004pU007pC004pU007 | | U004pU007pG004pC004pC004pU | |
| | | pG007pA007pA004pG004pG004 | | 004pU004pC004pA004pG007pA0 | |
| | | pC004pA004pA004pA004pU004 | | 04pG007pG004pU004pG004pA00 | |
| | | pA004pU004 | | 4pG004p001C004p001U004 | |
| 613 | 3102 | A004p001A004p001A004pA004 | 1019 | A004p001A007p001G004pA004p | 1260 |
| | | pG004pU004pU007pU004pU007 | | A004pU007pU004pU004pC004pA | |
| | | pG007pA007pU004pG004pA004 | | 004pU004pC004pA004pA007pA0 | |
| | | pA004pA004pU004pU004pC004 | | 04pA007pC004pU004pU004pU00 | |
| | | pU004pU004 | | 4pU004p001U004p001U004 | |
| 614 | 3105 | A004p001G004p001U004pU004 | 1020 | U004p001U007p001C004pA004p | 1261 |
| | | pU004pU004pG007pA004pU007 | | A004pG007pA004pA004pU004pU | |
| | | pG007pA007pA004pA004pU004 | | 004pU004pC004pA004pU007pC0 | |
| | | pU004pC004pU004pU004pG004 | | 04pA007pA004pA004pA004pC00 | |
| | | pA004pA004 | | 4pU004p001U004p001U004 | |
| 615 | 3108 | U004p001U004p001U004pG004 | 1021 | A004p001A007p001C004pU004p | 1262 |
| | | pA004pU004pG007pA004pA007 | | U004pC007pA004pA004pG004pA | |
| | | pA007pU007pU004pC004pU004 | | 004pA004pU004pU004pU007pC0 | |
| | | pU004pG004pA004pA004pG004 | | 04pA007pU004pC004pA004pA00 | |
| | | pU004pU004 | | 4pA004p001A004p001C004 | |
| 616 | 3111 | G004p001A004p001U004pG004 | 1022 | A004p001U007p001G004pA004p | 1263 |
| | | pA004pA004pA007pU004pU007 | | A004pC007pU004pU004pC004pA | |
| | | pC007pU007pU004pG004pA004 | | 004pA004pG004pA004pA007pU0 | |
| | | pA004pG004pU004pU004pC004 | | 04pU007pU004pC004pA004pU00 | |
| | | pA004pU004 | | 4pC004p001A004p001A004 | |
| 617 | 3117 | A004p001U004p001U004pC004 | 1023 | A004p001U007p001C004pA004p | 1264 |
| | | pU004pU004pG007pA004pA007 | | C004pC007pA004pU004pG004pA | |
| | | pG007pU007pU004pC004pA004 | | 004pA004pC004pU004pU007pC0 | |
| | | pU004pG004pG004pU004pG004 | | 04pA007pA004pG004pA004pA00 | |
| | | pA004pU004 | | 4pU004p001U004p001U004 | |
| 618 | 3126 | G004p001U004p001U004pC004 | 1024 | A004p001U007p001U004pG004p | 1265 |
| | | pA004pU004pG007pG004pU007 | | C004pA007pC004pU004pG004pA | |
| | | pG007pA007pU004pC004pA004 | | 004pU004pC004pA004pC007pC0 | |
| | | pG004pU004pG004pC004pA004 | | 04pA007pU004pG004pA004pA00 | |
| | | pA004pU004 | | 4pC004p001U004p001U004 | |
| 619 | 3129 | C004p001A004p001U004pG004 | 1025 | U004p001C007p001A004pA004p | 1266 |
| | | pG004pU004pG007pA004pU007 | | U004pU007pG004pC004pA004pC | |
| | | pC007pA007pG004pU004pG004 | | 004pU004pG004pA004pU007pC0 | |
| | | pC004pA004pA004pU004pU004 | | 04pA007pC004pC004pA004pU00 | |
| | | pG004pA004 | | 4pG004p001A004p001A004 | |
| 620 | 3130 | A004p001U004p001G004pG004 | 1026 | U004p001U007p001C004pA004p | 1267 |
| | | pU004pG004pA007pU004pC007 | | A004pU007pU004pG004pC004pA | |
| | | pA007pG007pU004pG004pC004 | | 004pC004pU004pG004pA007pU0 | |
| | | pA004pA004pU004pU004pG004 | | 04pC007pA004pC004pC004pA00 | |
| | | pA004pA004 | | 4pU004p001G004p001A004 | |
| 621 | 3208 | A004p001A004p001A004pC004 | 1027 | U004p001A007p001A004pC004p | 1268 |
| | | pU004pC004pC007pU004pG007 | | U004pA007pC004pA004pA004pA | |
| | | pA007pU007pU004pU004pU004 | | 004pA004pU004pC004pA007pG0 | |
| | | pG004pU004pA004pG004pU004 | | 04pG007pA004pG004pU004pU00 | |
| | | pU004pA004 | | 4pU004p001C004p001A004 | |
| 622 | 3212 | U004p001C004p001C004pU004 | 1028 | A004p001A007p001A004pU004p | 1269 |
| | | pG004pA004pU007pU004pU007 | | U004pA007pA004pC004pU004pA | |
| | | pU007pG007pU004pA004pG004 | | 004pC004pA004pA004pA007pA0 | |
| | | pU004pU004pA004pA004pU004 | | 04pU007pC004pA004pG004pG00 | |
| | | pU004pU004 | | 4pA004p001G004p001U004 | |
| 623 | 3213 | C004p001C004p001U004pG004 | 1029 | U004p001A007p001A004pA004p | 1270 |
| | | pA004pU004pU007pU004pU007 | | U004pU007pA004pA004pC004pU | |
| | | pG007pU007pA004pG004pU004 | | 004pA004pC004pA004pA007pA0 | |
| | | pU004pA004pA004pU004pU004 | | 04pA007pU004pC004pA004pG00 | |
| | | pU004pA004 | | 4pG004p001A004p001G004 | |
| 624 | 3229 | A004p001U004p001U004pU004 | 1030 | U004p001A007p001C004pA004p | 1271 |
| | | pA004pU004pU007pA004pA007 | | U004pC007pC004pC004pA004pG | |
| | | pG007pU007pC004pU004pG004 | | 004pA004pC004pU004pU007pA0 | |
| | | pG004pG004pA004pU004pG004 | | 04pA007pU004pA004pA004pA00 | |
| | | pU004pA004 | | 4pU004p001U004p001A004 | |
| 625 | 3230 | U004p001U004p001U004pA004 | 1031 | U004p001U007p001A004pC004p | 1272 |
| | | pU004pU004pA007pA004pG007 | | A004pU007pC004pC004pC004pA | |
| | | pU007pC007pU004pG004pG004 | | 004pG004pA004pC004pU007pU0 | |
| | | pG004pA004pU004pG004pU004 | | 04pA007pA004pU004pA004pA00 | |
| | | pA004pA004 | | 4pA004p001U004p001U004 | |
| 626 | 3256 | C004p001A004p001A004pG004 | 1032 | U004p001A007p001A004pC004p | 1273 |
| | | pA004pA004pG007pU004pA007 | | U004pU007pA004pG004pC004pU | |
| | | pA007pG007pA004pG004pC004 | | 004pC004pU004pU004pA007pC0 | |
| | | pU004pA004pA004pG004pU004 | | 04pU007pU004pC004pU004pU00 | |
| | | pU004pA004 | | 4pG004p001A004p001A004 | |
| 627 | 3460 | A004p001U004p001U004pU004 | 1033 | A004p001A007p001A004pU004p | 1274 |
| | | pA004pU004pC007pU004pC007 | | A004pG007pC004pC004pU004pG | |
| | | pC007pG007pC004pA004pG004 | | 004pC004pG004pG004pA007pG0 | |
| | | pG004pC004pU004pA004pU004 | | 04pA007pU004pA004pA004pA00 | |
| | | pU004pU004 | | 4pU004p001A004p001C004 | |
| 628 | 3465 | U004p001C004p001U004pC004 | 1034 | U004p001G007p001A004pA004p | 1275 |
| | | pC004pG004pC007pA004pG007 | | C004pA007pA004pA004pU004pA | |
| | | pG007pC007pU004pA004pU004 | | 004pG004pC004pC004pU007pG0 | |
| | | pU004pU004pG004pU004pU004 | | 04pC007pG004pG004pA004pG00 | |
| | | pC004pA004 | | 4pA004p001U004p001A004 | |
| 629 | 3466 | C004p001U004p001C004pC004 | 1035 | U004p001U007p001G004pA004p | 1276 |
| | | pG004pC004pA007pG004pG007 | | A004pC007pA004pA004pA004pU | |
| | | pc007pU007pA004pU004pU004 | | 004pA004pG004pC004pC007pU0 | |
| | | pU004pG004pU004pU004pC004 | | 04pG007pC004pG004pG004pA00 | |
| | | pA004pA004 | | 4pG004p001A004p001U004 | |
| 630 | 3482 | U004p001U004p001C004pA004 | 1036 | U004p001A007p001A004pA004p | 1277 |
| | | pG004pA004pG007pA004pG007 | | C004pA007pA004pA004pA004pG | |
| | | pG007pC007pC004pU004pU004 | | 004pG004pC004pC004pU007pC0 | |
| | | pU004pU004pG004pU004pU004 | | 04pU007pC004pU004pG004pA00 | |
| | | pU004pA004 | | 4pA004p001C004p001A004 | |
| 631 | 3484 | C004p001A004p001G004pA004 | 1037 | U004p001U007p001U004pA004p | 1278 |
| | | pG004pA004pG007pG004pC007 | | A004pA007pC004pA004pA004pA | |
| | | pC007pU007pU004pU004pU004 | | 004pA004pG004pG004pC007pC0 | |
| | | pG004pU004pU004pU004pA004 | | 04pU007pC004pU004pC004pU00 | |
| | | pA004pA004 | | 4pG004p001A004p001A004 | |
| 632 | 3487 | A004p001G004p001A004pG004 | 1038 | A004p001U007p001A004pU004p | 1279 |
| | | pG004pC004pC007pU004pU007 | | U004pU007pA004pA004pA004pC | |
| | | pU007pU007pG004pU004pU004 | | 004pA004pA004pA004pA007pG0 | |
| | | pU004pA004pA004pA004pU004 | | 04pG007pC004pC004pU004pC00 | |
| | | pA004pU004 | | 4pU004p001C004p001U004 | |
| 633 | 3532 | C004p001U004p001G004pG004 | 1039 | U004p001A007p001C004pA004p | 1280 |
| | | pA004pU004pU007pG004pG007 | | U004pG007pU004pU004pA004pU | |
| | | pC007pU007pA004pU004pA004 | | 004pA004pG004pC004pC007pA0 | |
| | | pA004pC004pA004pU004pG004 | | 04pA007pU004pC004pC004pA00 | |
| | | pU004pA004 | | 4pG004p001A004p001C004 | |
| 634 | 3533 | U004p001G004p001G004pA004 | 1040 | A004p001G007p001A004pC004p | 1281 |
| | | pU004pU004pG007pG004pC007 | | A004pU007pG004pU004pU004pA | |
| | | pU007pA007pU004pA004pA004 | | 004pU004pA004pG004pC007pC0 | |
| | | pC004pA004pU004pG004pU004 | | 04pA007pA004pU004pC004pC00 | |
| | | pC004pU004 | | 4pA004p001G004p001A004 | |
| 635 | 3537 | U004p001U004p001G004pG004 | 1041 | U004p001G007p001A004pA004p | 1282 |
| | | pC004pU004pA007pU004pA007 | | A004pG007pA004pC004pA004pU | |
| | | pA007pC007pA004pU004pG004 | | 004pG004pU004pU004pA007pU0 | |
| | | pU004pC004pU004pU004pU004 | | 04pA007pG004pC004pC004pA00 | |
| | | pC004pA004 | | 4pA004p001U004p001C004 | |
| 636 | 3654 | C004p001A004p001A004pG004 | 1042 | A004p001U007p001U004pU004p | 1283 |
| | | pA004pC004pU007pG004pG007 | | C004pU007pA004pA004pG004pG | |
| | | pG007pA007pC004pC004pU004 | | 004pU004pC004pC004pC007pA0 | |
| | | pU004pA004pG004pA004pA004 | | 04pG007pU004pC004pU004pU00 | |
| | | pA004pU004 | | 4pG004p001C004p001U004 | |
| 637 | 3655 | A004p001A004p001G004pA004 | 1043 | U004p001A007p001U004pU004p | 1284 |
| | | pC004pU004pG007pG004pG007 | | U004pC007pU004pA004pA004pG | |
| | | pA007pC007pC004pU004pU004 | | 004pG004pU004pC004pC007pC0 | |
| | | pA004pG004pA004pA004pA004 | | 04pA007pG004pU004pC004pU00 | |
| | | pU004pA004 | | 4pU004p001G004p001C004 | |
| 638 | 3714 | U004p001C004p001U004pA004 | 1044 | A004p001G007p001C004pA004p | 1285 |
| | | pA004pG004pC007pC004pA007 | | A004pU007pU004pA004pA004pA | |
| | | pA007pC007pU004pU004pU004 | | 004pG004pU004pU004pG007pG0 | |
| | | pA004pA004pU004pU004pG004 | | 04pC007pU004pU004pA004pG00 | |
| | | pC004pU004 | | 4pA004p001G004p001A004 | |
| 639 | 3773 | U004p001U004p001U004pG004 | 1045 | A004p001G007p001A004pU004p | 1286 |
| | | pU004pA004pA007pA004pC007 | | U004pU007pG004pA004pU004pA | |
| | | pU007pG007pU004pA004pU004 | | 004pC004pA004pG004pU007pU0 | |
| | | pC004pA004pA004pA004pU004 | | 04pU007pA004pC004pA004pA00 | |
| | | pC004pU004 | | 4pA004p001A004p001A004 | |
| 640 | 3784 | U004p001A004p001U004pC004 | 1046 | A004p001C007p001A004pA004p | 1287 |
| | | pA004pA004pA007pU004pC007 | | C004pA007pU004pA004pU004pA | |
| | | pU007pG007pU004pA004pU004 | | 004pC004pA004pG004pA007pU0 | |
| | | pA004pU004pG004pU004pU004 | | 04pU007pU004pG004pA004pU00 | |
| | | pG004pU004 | | 4pA004p001C004p001A004 | |
| 641 | 3814 | U004p001A004p001U004pG004 | 1047 | U004p001A007p001C004pU004p | 1288 |
| | | pC004pU004pA007pG004pU007 | | U004pC007pC004pA004pA004pU | |
| | | pU007pU007pA004pU004pU004 | | 004pA004pA004pA004pC007pU0 | |
| | | pG004pG004pA004pA004pG004 | | 04pA007pG004pC004pA004pU00 | |
| | | pU004pA004 | | 4pA004p001A004p001G004 | |
| 642 | 3825 | A004p001U004p001U004pG004 | 1048 | U004p001A007p001U004pU004p | 1289 |
| | | pG004pA004pA007pG004pU007 | | U004pC007pU004pU004pG004pA | |
| | | pG007pU007pU004pC004pA004 | | 004pA004pC004pA004pC007pU0 | |
| | | pA004pG004pA004pA004pA004 | | 04pU007pC004pC004pA004pA00 | |
| | | pU004pA004 | | 4pU004p001A004p001A004 | |
| 643 | 3827 | U004p001G004p001G004pA004 | 1049 | U004p001U007p001U004pA004p | 1290 |
| | | pA004pG004pU007pG004pU007 | | U004pU007pU004pC004pU004pU | |
| | | pU007pC007pA004pA004pG004 | | 004pG004pA004pA004pC007pA0 | |
| | | pA004pA004pA004pU004pA004 | | 04pC007pU004pU004pC004pC00 | |
| | | pA004pA004 | | 4pA004p001A004p001U004 | |
| 644 | 3850 | U004p001C004p001A004pA004 | 1050 | U004p001U007p001U004pU004p | 1291 |
| | | pC004pU004pU007pG004pU007 | | A004pU007pC004pA004pG004pU | |
| | | pG007pU007pA004pC004pU004 | | 004pA004pC004pA004pC007pA0 | |
| | | pG004pA004pU004pA004pA004 | | 04pA007pG004pU004pU004pG00 | |
| | | pA004pA004 | | 4pA004p001U004p001U004 | |
| 645 | 4097 | G004p001U004p001C004pA004 | 1051 | A004p001G007p001A004pU004p | 1292 |
| | | pG004pC004pA007pG004pA007 | | U004pC007pA004pA004pU004pA | |
| | | pG007pU007pU004pA004pU004 | | 004pA004pC004pU004pC007pU0 | |
| | | pU004pG004pA004pA004pU004 | | 04pG007pC004pU004pG004pA00 | |
| | | pC004pU004 | | 4pC004p001C004p001C004 | |
| 646 | 4120 | A004p001U004p001U004pU004 | 1052 | A004p001A007p001C004pU004p | 1293 |
| | | pU004pU004pU007pU004pU007 | | U004pG007pU004pA004pC004pA | |
| | | pA007pA007pU004pG004pU004 | | 004pU004pU004pA004pA007pA0 | |
| | | pA004pC004pA004pA004pG004 | | 04pA007pA004pA004pA004pA00 | |
| | | pU004pU004 | | 4pU004p001U004p001A004 | |

Table A below shows codes in the nucleotide sequences in Table 2 and the following Tables in the disclosure.

TABLE A

| | |
|---|---|
| p: phosphate group/phosphodiester linkage | |
| p001: phosphorothioate linker(PS) | |
| SS: sense strand | AS: antisense strand |
| B001: abasic nucleoside | |
| A000: unmodified adenosine (A) | G000: unmodified guanosine (G) |
| A002: deoxyriboadenosine (dA) | G002: deoxyriboguanosine (dG) |
| A004: adenosine/2′ OMe | G004: guanosine/2′ OMe |
| A005: adenosine/2′ MOE | G005: guanosine/2′ MOE |
| A007: adenosine/2′ F | G007: guanosine/2′ F |
| A1016: GNA with adenine | G1016: GNA with guanine |
| A042: TNA with adenine | G042: TNA with guanine |
| A1017: locked nucleotide with adenine | G1017: locked nucleotide with guanine |
| X033A1027: adenosine/5′(E)-VP-2′-OMe | X033G1027: guanosine/5′(E)-VP-2′-OMe |
| C000: unmodified cytidine (C) | U000: unmodified uridine (U) |
| C002: deoxyribocytidine (dC) | T000: ribothymidine |
| C004: cytidine/2′ OMe | T002: deoxyribothymidine (dT) |
| C005 or C005*: 5-methyl-cytidine/2′ MOE | U004: uridine/2′ OMe |
| C007: cytidine/2′ F | T005: ribothymidine (5-methyl uridine)/2′ MOE |
| C1016: GNA with cytosine | U007: uridine/2′ F |
| C042: TNA with cytosine | U1016: GNA with uracil |
| C1017: locked nucleotide with cytosine | U042: TNA with uracil |
| X033C1027: cytidine/5′(E)-VP-2′-OMe | U1017: locked nucleotide with uracil |
| | X033U1027: uridine/5′(E)-VP-2′-OMe |

The sequences and sequence lists including modified nucleosides (e.g., RNA, RNA modified at a 2′-OH sugar moiety, or RNA modified at a nucleobase) in the disclosure (e.g., Tables 5-8, 10, and 14) are indicated with codes defined in Table A unless otherwise indicated. Each code consists of a letter representing a type of the nucleobase, e.g., “A”, “G”, “C”, “U,” or “T” and a numeric code representing a type of modification on a sugar ring.

For example, if a nucleoside is coded as “T005”, it is meant by a RNA nucleoside including a 2′-MOE sugar moiety and a thymine (or methylated uracil) as “T” indicates a thymine (or methylated uracil) nucleobase and “005” indicates a 2′-MOE substituent at 2′-OH position on the sugar ring.

In particular example, if a nucleoside is coded as “C005*”, it is meant by a RNA nucleoside including a 5-methylated cytosine and a 2′-MOE sugar moiety as “C” indicates a type of specific nucleobase, i.e. 5-methylated cytosine, that can exist in combination with a 2′-MOE sugar moiety and “005” indicates a 2′-MOE substituent at 2′-OH position on the sugar ring.

The nucleosides in each sequence of the dsRNA are connected via phosphodiester group (“p” in Table A) or modification thereof (e.g., phosphorothioate linkage “p001” in Table A). In some embodiments, an example nucleotide may include a nucleoside and 3′-phosphodiester group. Example nucleotides may be presented in in Table A-1.

TABLE A-1

| Code for | | |
|---|---|---|
| nucleotide | 5′-end first nucleotide | Other position |
| A004p: 2′-O-methyl adenosine-3′- phosphate | 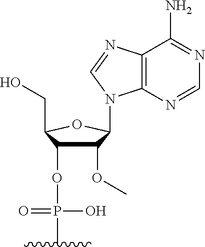 | 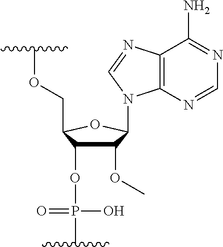 |
| U004p: 2′-O-methyl uridine-3′- phosphate | 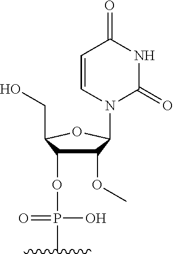 | 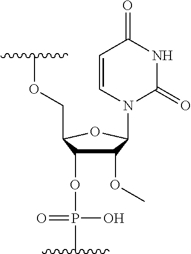 |
| C004p: 2′-O-methyl cytidine-3′- phosphate | 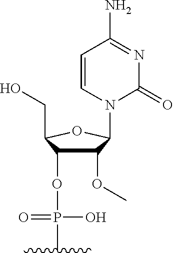 | 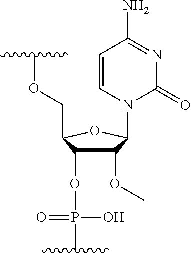 |
| G004p: 2′-O-methyl guanosine-3′- phosphate | 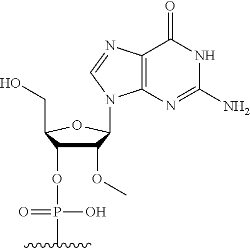 | 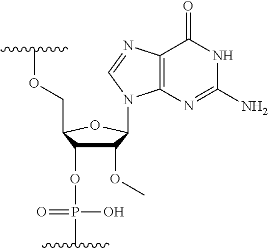 |
| A004p001: 2′-O-methyl adenosine-3′- phosphorothioate | 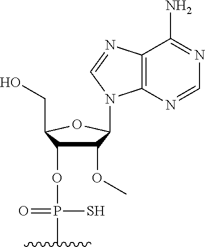 | 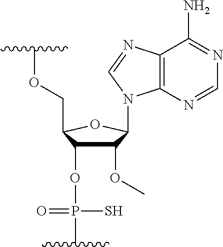 |
| U004p001: 2′-O-methyl uridine-3′- phosphorothioate | 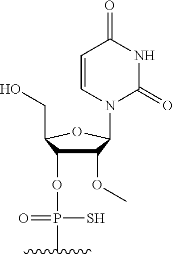 | 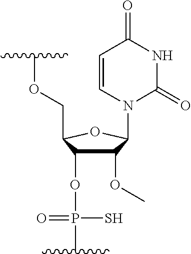 |
| C004p001: 2′-O-methyl cytidine-3′- phosphorothioate | 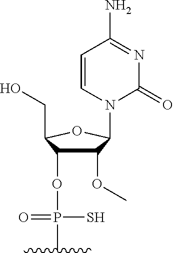 | 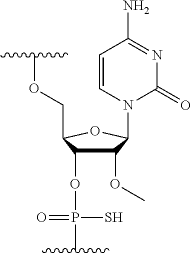 |
| G004p001: 2′-O-methyl guanosine-3′- phosphorothioate | 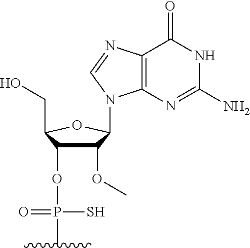 | 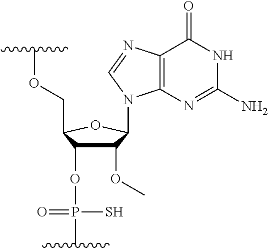 |
| A005p: 2′-O- methoxyethyl (MOE) adenosine-3′- phosphate | 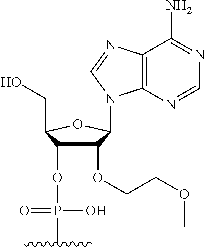 | 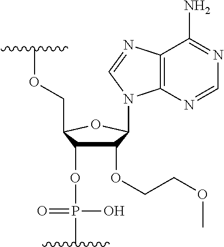 |
| T005p: 2′-O-methoxyethyl (MOE) thymidine (or 5- methyl uridine)-3′- phosphate | 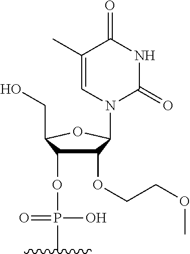 | 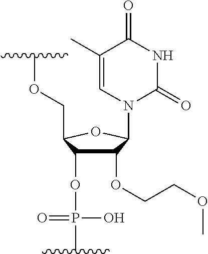 |
| C005*p: 2′-O-methoxyethyl (MOE) 5-methyl-cytidine- 3′-phosphate | 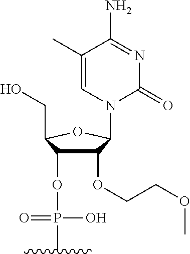 | 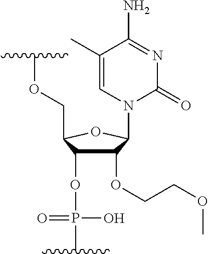 |
| G005p: 2′-O-methoxyethyl (MOE) guanosine-3′- phosphate | 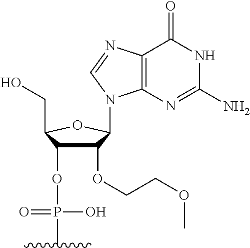 | 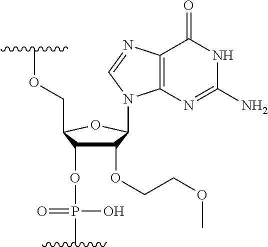 |
| A005p001: 2′-O- methoxyethyl (MOE) adenosine-3′- phosphorothioate | 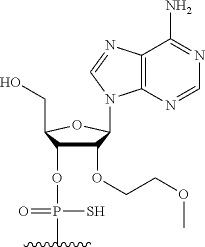 | 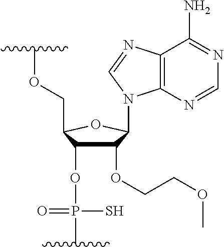 |
| T005p001: 2′-O-methoxyethyl (MOE) thymidine (or 5- methyl uridine)-3′- phosphorothioate | 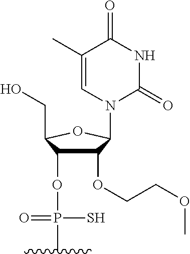 | 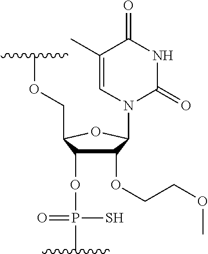 |
| C005*p001: 2′-O-methoxyethyl (MOE) 5-methyl-cytidine- 3′- phosphorothioate | 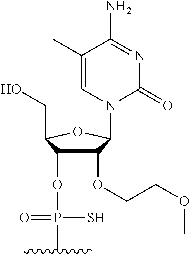 | 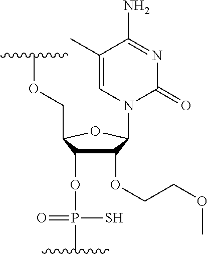 |
| G005p001: 2′-O-methoxyethyl (MOE) guanosine-3′- phosphorothioate | 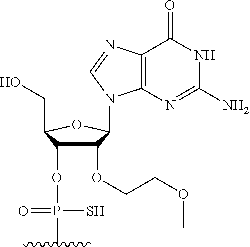 | 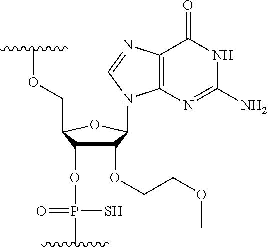 |
| A007p: 2′-fluoro adenosine-3′- phosphate | 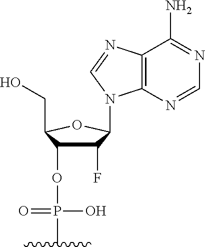 | 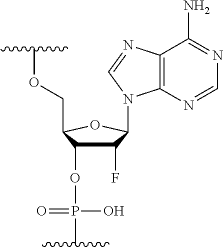 |
| U007p: 2′-fluoro uridine-3′- phosphate | 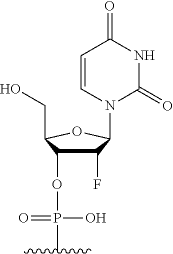 | 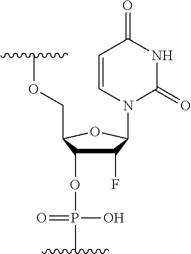 |
| C007p: 2′-fluoro cytidine-3′- phosphate | 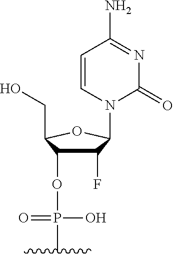 | 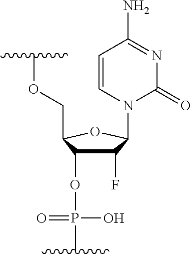 |
| G007p: 2′-fluoro guanosine-3′- phosphate | 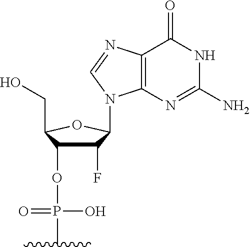 | 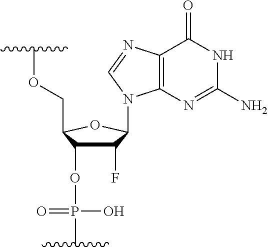 |
| A007p001: 2′-fluoro adenosine-3′- phosphorothioate | 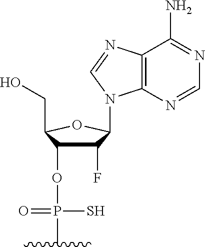 | 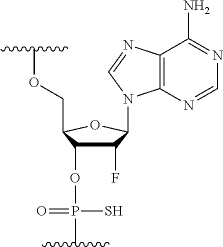 |
| U007p001: 2′-fluoro uridine-3′- phosphorothioate | 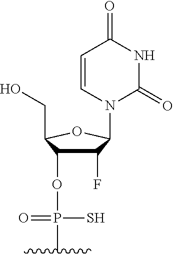 | 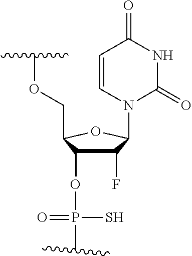 |
| C007p001: 2′-fluoro cytidine-3′- phosphorothioate | 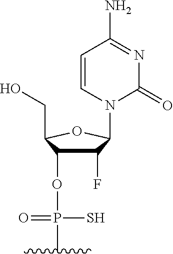 | 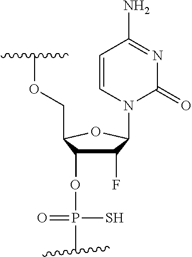 |
| G007p001: 2′-fluoro guanosine-3′- phosphorothioate | 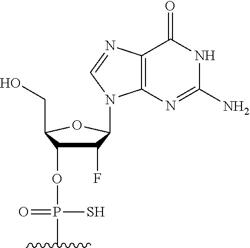 | 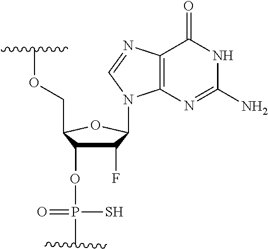 |
| T002p: 2′- deoxyribothymidine- 3′-phosphate | 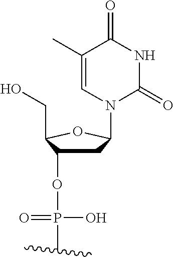 | 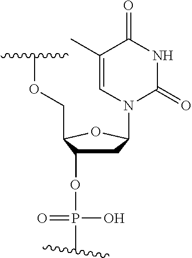 |
| T002p001: 2′- deoxythymidine- 3′- phosphorothioate | 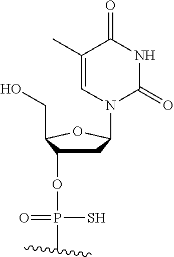 | 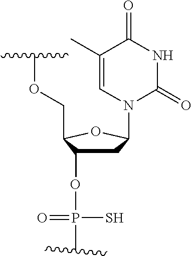 |
| A1016p: GNA with adenine-2′- phosphate | 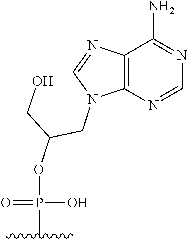 [SMILES: *P(=O)(O)OC(CO)Cn1cnc2c(N)ncnc21] | 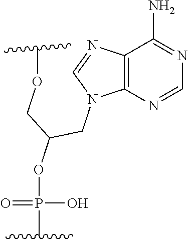 [SMILES: *OCC(Cn1cnc2c(N)ncnc21)OP(*)(=O)O] |
| U1016p: GNA with uracil- 2′-phosphate | 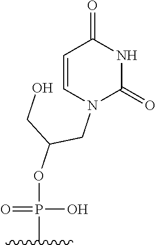 [SMILES: *P(=O)(O)OC(CO)Cn1ccc(=O)[nH]c1=O] | 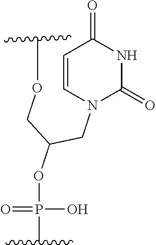 [SMILES: *OCC(Cn1ccc(=O)[nH]c1=O)OP(*)(=O)O] |
| C1016p: GNA with cytosine-2′- phosphate | 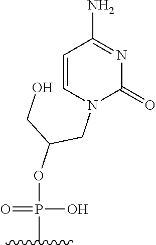 [SMILES: *P(=O)(O)OC(CO)Cn1ccc(N)nc1=O] | 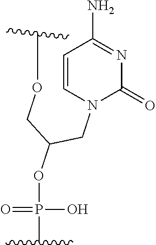 [SMILES: *OCC(Cn1ccc(N)nc1=O)OP(*)(=O)O] |
| G1016p: GNA with guanine-2′- phosphate | 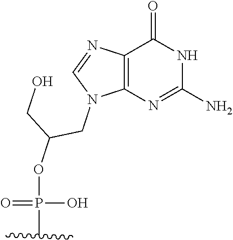 [SMILES: *P(=O)(O)OC(CO)Cn1cnc2c(=O)[nH]c(N)nc21] | 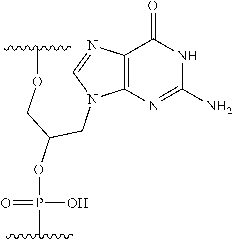 [SMILES: *OCC(Cn1cnc2c(=O)[nH]c(N)nc21)OP(*)(=O)O] |
| A042p: TNA with adenine-2′- phosphate | 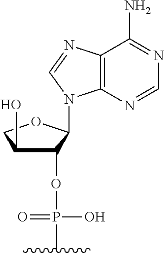 | 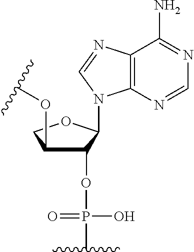 |
| U042p: TNA with uracil-2′- phosphate | 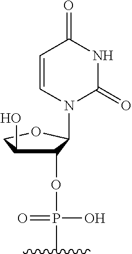 | 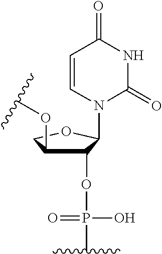 |
| C042p: TNA with cytosine-2′- phosphate | 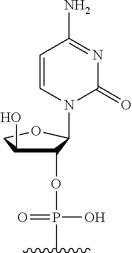 | 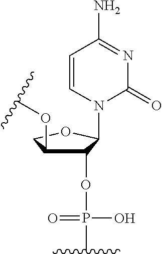 |
| G042p: TNA with guanine-2′- phosphate | 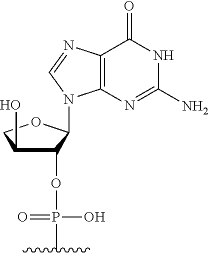 | 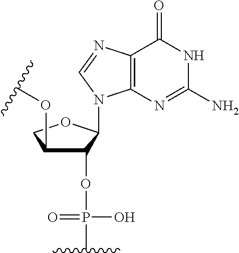 |
| A042p001: TNA with adenine-2′- phosphorothioate | 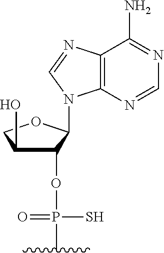 | 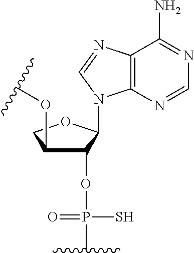 |
| U042p001: TNA with uracil-2′- phosphorothioate | 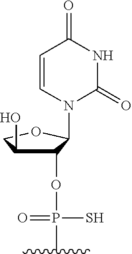 | 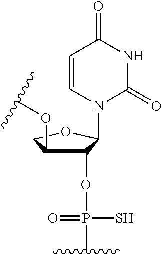 |
| C042p001: TNA with cytosine-2′- phosphorothioate | 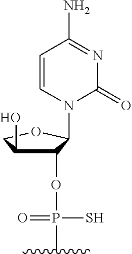 | 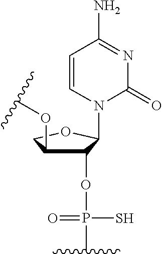 |
| G042p001: TNA with guanine-2′- phosphate | 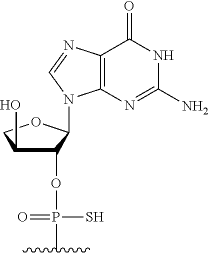 | 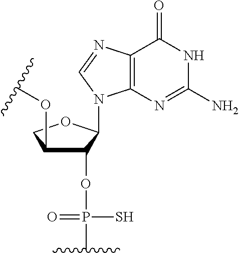 |
| X033A1027p001: 5′-(E)- vinylphosphonate- 2′-O-methyl adenosine-3′- phosphorothioate | 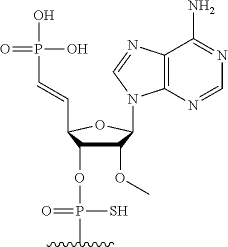 | |
| X033U1027p001: 5′-(E)- vinylphosphonate- 2′-O-methyl uridine-3′- phosphorothioate | 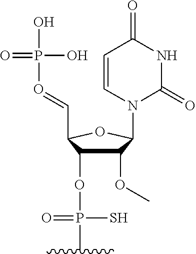 | |
| X033C1027p001: 5′-(E)- vinylphosphonate- 2′-O-methyl cytidine-3′- phosphorothioate | 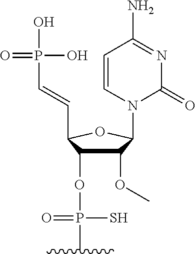 | |
| X033G1027p001: 5′-(E)- vinylphosphonate- 2′-O-methyl guanosine-3′- phosphorothioate | 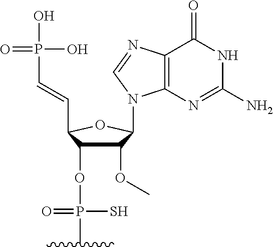 | |

In some embodiments, the dsRNA includes a sense strand having 10 contiguous nucleotides differing by no more than 3 nucleotides from the nucleotide sequence selected from SEQ ID NOs: 812 to 1052. In some embodiments, the dsRNA includes an antisense strand having 10 contiguous nucleotides differing by no more than 3 nucleotides from the nucleotide sequence of SEQ ID NOs: 1053 to 1293. In some embodiments, the dsRNA includes a sense strand having 11 contiguous nucleotides differing by no more than 3 nucleotides from the nucleotide sequence selected from SEQ ID NOs: 812 to 1052. In some embodiments, the dsRNA includes an antisense strand having 11 contiguous nucleotides differing by no more than 3 nucleotides from the nucleotide sequence selected from SEQ ID NOs: 1053 to 1293. In some embodiments, the dsRNA includes a sense strand having 12 contiguous nucleotides differing by no more than 3 nucleotides from the nucleotide sequence selected from SEQ ID NOs: 812 to 1052. In some embodiments, the dsRNA includes an antisense strand having 12 contiguous nucleotides differing by no more than 3 nucleotides from the nucleotide sequence selected from SEQ ID NOs: 1053 to 1293. In some embodiments, the dsRNA includes a sense strand having 13 contiguous nucleotides differing by no more than 3 nucleotides from the nucleotide sequence selected from SEQ ID NOs: 812 to 1052. In some embodiments, the dsRNA includes an antisense strand having 13 contiguous nucleotides differing by no more than 3 nucleotides from the nucleotide sequence selected from SEQ ID NOs: 1053 to 1293. In some embodiments, the dsRNA includes a sense strand having 14 contiguous nucleotides differing by no more than 3 nucleotides from the nucleotide sequence selected from SEQ ID NOs: 812 to 1052. In some embodiments, the dsRNA includes an antisense strand having 14 contiguous nucleotides differing by no more than 3 nucleotides from the nucleotide sequence selected from SEQ ID NOs: 1053 to 1293. In some embodiments, the dsRNA includes a sense strand having 15 contiguous nucleotides differing by no more than 3 nucleotides from the nucleotide sequence selected from SEQ ID NOs: 812 to 1052. In some embodiments, the dsRNA includes an antisense strand having 15 contiguous nucleotides differing by no more than 3 nucleotides from the nucleotide sequence selected from SEQ ID NOs: 1053 to 1293. In some embodiments, the dsRNA includes a sense strand having 16 contiguous nucleotides differing by no more than 3 nucleotides from the nucleotide sequence selected from SEQ ID NOs: 812 to 1052. In some embodiments, the dsRNA includes an antisense strand having 16 contiguous nucleotides differing by no more than 3 nucleotides from the nucleotide sequence selected from SEQ ID NOs: 1053 to 1293. In some embodiments, the dsRNA includes a sense strand having 17 contiguous nucleotides differing by no more than 3 nucleotides from the nucleotide sequence selected from SEQ ID NOs: 812 to 1052. In some embodiments, the dsRNA includes an antisense strand having 17 contiguous nucleotides differing by no more than 3 nucleotides from the nucleotide sequence selected from SEQ ID NOs: 1053 to 1293. In some embodiments, the dsRNA includes a sense strand having 18 contiguous nucleotides differing by no more than 3 nucleotides from the nucleotide sequence selected from SEQ ID NOs: 812 to 1052. In some embodiments, the dsRNA includes an antisense strand having 18 contiguous nucleotides differing by no more than 3 nucleotides from the nucleotide sequence selected from SEQ ID NOs: 1053 to 1293. In some embodiments, the dsRNA includes a sense strand having 19 contiguous nucleotides differing by no more than 3 nucleotides from the nucleotide sequence selected from SEQ ID NOs: 812 to 1052. In some embodiments, the dsRNA includes an antisense strand having 19 contiguous nucleotides differing by no more than 3 nucleotides from the nucleotide sequence selected from SEQ ID NOs: 1053 to 1293. In some embodiments, the dsRNA includes a sense strand having 20 contiguous nucleotides differing by no more than 3 nucleotides from the nucleotide sequence selected from SEQ ID NOs: 812 to 1052. In some embodiments, the dsRNA includes an antisense strand having 20 contiguous nucleotides differing by no more than 3 nucleotides from the nucleotide sequence selected from SEQ ID NOs: 1053 to 1293. In some embodiments, the dsRNA includes a sense strand having 21 contiguous nucleotides differing by no more than 3 nucleotides from the nucleotide sequence selected from SEQ ID NOs: 812 to 1052. In some embodiments, the dsRNA includes an antisense strand having 21 contiguous nucleotides differing by no more than 3 nucleotides from the nucleotide sequence selected from SEQ ID NOs: 1053 to 1293.

In some embodiments, the dsRNA includes a sense strand having 10 contiguous nucleotides differing by no more than 2 nucleotides from the nucleotide sequence selected from SEQ ID NOs: 812 to 1052. In some embodiments, the dsRNA includes an antisense strand having 10 contiguous nucleotides differing by no more than 2 nucleotides from the nucleotide sequence selected from SEQ ID NOs: 1053 to 1293. In some embodiments, the dsRNA includes a sense strand having 11 contiguous nucleotides differing by no more than 2 nucleotides from the nucleotide sequence selected from SEQ ID NOs: 812 to 1052. In some embodiments, the dsRNA includes an antisense strand having 11 contiguous nucleotides differing by no more than 2 nucleotides from the nucleotide sequence selected from SEQ ID NOs: 1053 to 1293. In some embodiments, the dsRNA includes a sense strand having 12 contiguous nucleotides differing by no more than 2 nucleotides from the nucleotide sequence selected from SEQ ID NOs: 812 to 1052. In some embodiments, the dsRNA includes an antisense strand having 12 contiguous nucleotides differing by no more than 2 nucleotides from the nucleotide sequence selected from SEQ ID NOs: 1053 to 1293. In some embodiments, the dsRNA includes a sense strand having 13 contiguous nucleotides differing by no more than 2 nucleotides from the nucleotide sequence selected from SEQ ID NOs: 812 to 1052. In some embodiments, the dsRNA includes an antisense strand having 13 contiguous nucleotides differing by no more than 2 nucleotides from the nucleotide sequence selected from SEQ ID NOs: 1053 to 1293. In some embodiments, the dsRNA includes a sense strand having 14 contiguous nucleotides differing by no more than 2 nucleotides from the nucleotide sequence selected from SEQ ID NOs: 812 to 1052. In some embodiments, the dsRNA includes an antisense strand having 14 contiguous nucleotides differing by no more than 2 nucleotides from the nucleotide sequence selected from SEQ ID NOs: 1053 to 1293. In some embodiments, the dsRNA includes a sense strand having 15 contiguous nucleotides differing by no more than 2 nucleotides from the nucleotide sequence selected from SEQ ID NOs: 812 to 1052. In some embodiments, the dsRNA includes an antisense strand having 15 contiguous nucleotides differing by no more than 2 nucleotides from the nucleotide sequence selected from SEQ ID NOs: 1053 to 1293. In some embodiments, the dsRNA includes a sense strand having 16 contiguous nucleotides differing by no more than 2 nucleotides from the nucleotide sequence selected from SEQ ID NOs: 812 to 1052. In some embodiments, the dsRNA includes an antisense strand having 16 contiguous nucleotides differing by no more than 2 nucleotides from the nucleotide sequence selected from SEQ ID NOs: 1053 to 1293. In some embodiments, the dsRNA includes a sense strand having 17 contiguous nucleotides differing by no more than 2 nucleotides from the nucleotide sequence selected from SEQ ID NOs: 812 to 1052. In some embodiments, the dsRNA includes an antisense strand having 17 contiguous nucleotides differing by no more than 2 nucleotides from the nucleotide sequence selected from SEQ ID NOs: 1053 to 1293. In some embodiments, the dsRNA includes a sense strand having 18 contiguous nucleotides differing by no more than 2 nucleotides from the nucleotide sequence selected from SEQ ID NOs: 812 to 1052. In some embodiments, the dsRNA includes an antisense strand having 18 contiguous nucleotides differing by no more than 2 nucleotides from the nucleotide sequence selected from SEQ ID NOs: 1053 to 1293. In some embodiments, the dsRNA includes a sense strand having 19 contiguous nucleotides differing by no more than 2 nucleotides from the nucleotide sequence selected from SEQ ID NOs: 812 to 1052. In some embodiments, the dsRNA includes an antisense strand having 19 contiguous nucleotides differing by no more than 2 nucleotides from the nucleotide sequence selected from SEQ ID NOs: 1053 to 1293. In some embodiments, the dsRNA includes a sense strand having 20 contiguous nucleotides differing by no more than 2 nucleotides from the nucleotide sequence selected from SEQ ID NOs: 812 to 1052. In some embodiments, the dsRNA includes an antisense strand having 20 contiguous nucleotides differing by no more than 2 nucleotides from the nucleotide sequence selected from SEQ ID NOs: 1053 to 1293. In some embodiments, the dsRNA includes a sense strand having 21 contiguous nucleotides differing by no more than 2 nucleotides from the nucleotide sequence selected from SEQ ID NOs: 812 to 1052. In some embodiments, the dsRNA includes an antisense strand having 21 contiguous nucleotides differing by no more than 2 nucleotides from the nucleotide sequence selected from SEQ ID NOs: 1053 to 1293.

In some embodiments, the dsRNA includes a sense strand having 10 contiguous nucleotides differing by no more than 1 nucleotide from the nucleotide sequence selected from SEQ ID NOs: 812 to 1052. In some embodiments, the dsRNA includes an antisense strand having 10 contiguous nucleotides differing by no more than 1 nucleotide from the nucleotide sequence selected from SEQ ID NOs: 1053 to 1293. In some embodiments, the dsRNA includes a sense strand having 11 contiguous nucleotides differing by no more than 1 nucleotide from the nucleotide sequence selected from SEQ ID NOs: 812 to 1052. In some embodiments, the dsRNA includes an antisense strand having 11 contiguous nucleotides differing by no more than 1 nucleotide from the nucleotide sequence selected from SEQ ID NOs: 1053 to 1293. In some embodiments, the dsRNA includes a sense strand having 12 contiguous nucleotides differing by no more than 1 nucleotide from the nucleotide sequence selected from SEQ ID NOs: 812 to 1052. In some embodiments, the dsRNA includes an antisense strand having 12 contiguous nucleotides differing by no more than 1 nucleotide from the nucleotide sequence selected from SEQ ID NOs: 1053 to 1293. In some embodiments, the dsRNA includes a sense strand having 13 contiguous nucleotides differing by no more than 1 nucleotide from the nucleotide sequence selected from SEQ ID NOs: 812 to 1052. In some embodiments, the dsRNA includes an antisense strand having 13 contiguous nucleotides differing by no more than 1 nucleotide from the nucleotide sequence selected from SEQ ID NOs: 1053 to 1293. In some embodiments, the dsRNA includes a sense strand having 14 contiguous nucleotides differing by no more than 1 nucleotide from the nucleotide sequence selected from SEQ ID NOs: 812 to 1052. In some embodiments, the dsRNA includes an antisense strand having 14 contiguous nucleotides differing by no more than 1 nucleotide from the nucleotide sequence selected from SEQ ID NOs: 1053 to 1293. In some embodiments, the dsRNA includes a sense strand having 15 contiguous nucleotides differing by no more than 1 nucleotide from the nucleotide sequence selected from SEQ ID NOs: 812 to 1052. In some embodiments, the dsRNA includes an antisense strand having 15 contiguous nucleotides differing by no more than 1 nucleotide from the nucleotide sequence selected from SEQ ID NOs: 1053 to 1293. In some embodiments, the dsRNA includes a sense strand having 16 contiguous nucleotides differing by no more than 1 nucleotide from the nucleotide sequence selected from SEQ ID NOs: 812 to 1052. In some embodiments, the dsRNA includes an antisense strand having 16 contiguous nucleotides differing by no more than 1 nucleotide from the nucleotide sequence selected from SEQ ID NOs: 1053 to 1293. In some embodiments, the dsRNA includes a sense strand having 17 contiguous nucleotides differing by no more than 1 nucleotide from the nucleotide sequence selected from SEQ ID NOs: 812 to 1052. In some embodiments, the dsRNA includes an antisense strand having 17 contiguous nucleotides differing by no more than 1 nucleotide from the nucleotide sequence selected from SEQ ID NOs: 1053 to 1293. In some embodiments, the dsRNA includes a sense strand having 18 contiguous nucleotides differing by no more than 1 nucleotide from the nucleotide sequence selected from SEQ ID NOs: 812 to 1052. In some embodiments, the dsRNA includes an antisense strand having 18 contiguous nucleotides differing by no more than 1 nucleotide from the nucleotide sequence selected from SEQ ID NOs: 1053 to 1293. In some embodiments, the dsRNA includes a sense strand having 19 contiguous nucleotides differing by no more than 1 nucleotide from the nucleotide sequence selected from SEQ ID NOs: 812 to 1052. In some embodiments, the dsRNA includes an antisense strand having 19 contiguous nucleotides differing by no more than 1 nucleotide from the nucleotide sequence selected from SEQ ID NOs: 1053 to 1293. In some embodiments, the dsRNA includes a sense strand having 20 contiguous nucleotides differing by no more than 1 nucleotide from the nucleotide sequence selected from SEQ ID NOs: 812 to 1052. In some embodiments, the dsRNA includes an antisense strand having 20 contiguous nucleotides differing by no more than 1 nucleotide from the nucleotide sequence selected from SEQ ID NOs: 1053 to 1293. In some embodiments, the dsRNA includes a sense strand having 21 contiguous nucleotides differing by no more than 1 nucleotide from the nucleotide sequence selected from SEQ ID NOs: 812 to 1052. In some embodiments, the dsRNA includes an antisense strand having 21 contiguous nucleotides differing by no more than 1 nucleotide from the nucleotide sequence selected from SEQ ID NOs: 1053 to 1293. In some embodiments, the dsRNA includes an antisense strand having 22 contiguous nucleotides differing by no more than 1 nucleotide from the nucleotide sequence selected from SEQ ID NOs: 1053 to 1293. In some embodiments, the dsRNA includes an antisense strand having 23 contiguous nucleotides differing by no more than 1 nucleotide from the nucleotide sequence selected from SEQ ID NOs: 1053 to 1293.

In some embodiments, the dsRNA includes (i) a sense strand having 15 contiguous nucleotides differing by no more than one, two or three nucleotides from the nucleotide sequence selected from SEQ ID NOs: 812 to 1052 and (ii) an antisense strand forming a duplex with the sense strand of (i) and having 15 contiguous nucleotides differing by no more than one, two or three nucleotides from the nucleotide sequence selected from SEQ ID NOs: 1053 to 1293. In some embodiments, the dsRNA includes (i) a sense strand having 16 contiguous nucleotides differing by no more than one, two or three from the nucleotide sequence selected from SEQ ID NOs: 812 to 1052 and (ii) an antisense strand forming a duplex with the sense strand of (i) and having 16 contiguous nucleotides differing by no more than one, two or three nucleotides from the nucleotide sequence selected from SEQ ID NOs: 1053 to 1293. In some embodiments, the dsRNA includes (i) a sense strand having 17 contiguous nucleotides differing by no more than one, two or three nucleotides from the nucleotide sequence selected from SEQ ID NOs: 812 to 1052 and (ii) an antisense strand forming a duplex with the sense strand of (i) and having 17 contiguous nucleotides differing by no more than one, two or three nucleotides from the nucleotide sequence selected from SEQ ID NOs: 1053 to 1293. In some embodiments, the dsRNA includes (i) a sense strand having 18 contiguous nucleotides differing by no more than one, two or three nucleotides from the nucleotide sequence selected from SEQ ID NOs: 812 to 1052 and (ii) an antisense strand forming a duplex with the sense strand of (i) and having 18 contiguous nucleotides differing by no more than one, two or three nucleotides from the nucleotide sequence selected from SEQ ID NOs: 1053 to 1293. In some embodiments, the dsRNA includes (i) a sense strand having 19 contiguous nucleotides differing by no more than one, two or three nucleotides from the nucleotide sequence selected from SEQ ID NOs: 812 to 1052 and (ii) an antisense strand forming a duplex with the sense strand of (i) and having 19 contiguous nucleotides differing by no more than one, two or three nucleotides from the nucleotide sequence selected from SEQ ID NOs: 1053 to 1293. In some embodiments, the dsRNA includes (i) a sense strand having 20 contiguous nucleotides differing by no more than one, two or three nucleotides from the nucleotide sequence selected from SEQ ID NOs: 812 to 1052 and (ii) an antisense strand forming a duplex with the sense strand of (i) and having 20 contiguous nucleotides differing by no more than one, two or three nucleotides from the nucleotide sequence selected from SEQ ID NOs: 1053 to 1293. In some embodiments, the dsRNA includes (i) a sense strand having 21 contiguous nucleotides differing by no more than one, two or three nucleotides from the nucleotide sequence selected from SEQ ID NOs: 812 to 1052 and (ii) an antisense strand forming a duplex with the sense strand of (i) and having 21 contiguous nucleotides differing by no more than one, two or three nucleotides from the nucleotide sequence selected from SEQ ID NOs: 1053 to 1293.

In certain aspects, when a sense strand or an antisense strand of a dsRNA in above paragraphs is differing by a certain number of nucleotides (e.g., one, two or three nucleotides) from a specific sequence (e.g., SEQ ID NOs: 812 to 1293), it is meant by that the sense strand or the antisense strand includes one, two or three nucleotides, having different nucleobases and/or different modifications compared to the nucleobases and/or the modifications of the nucleotides at the corresponding positions of the specific sequence (e.g., SEQ ID NOs: 812 to 1293). In some embodiments, when a sense strand or an antisense strand is differing by a certain number of nucleotides (e.g., one, two or three nucleotides) from a specific sequence (e.g., SEQ ID NOs: 812 to 1293), the sense strand or the antisense strand includes one, two, or three nucleotides, having different nucleobases compared to the nucleobases of the nucleotides at the corresponding positions of the specific sequence (e.g., SEQ ID NOs: 812 to 1293). In some embodiments, when a sense strand or an antisense strand is differing by a certain number of nucleotides (e.g., one, two or three nucleotides) from a specific sequence (e.g., SEQ ID NOs: 812 to 1293), the sense strand or the antisense strand includes one, two, or three nucleotides, having different modifications compared to the modifications of the nucleotides at the corresponding positions of the specific sequence (e.g., SEQ ID NOs: 812 to 1293). In some embodiments, when a sense strand or an antisense strand is differing by a certain number of nucleotides (e.g., one, two or three nucleotides) from a specific sequence (e.g., SEQ ID NOs: 812 to 1293), the sense strand or the antisense strand includes one, two, or three nucleotides having different nucleobases and different modifications compared to the nucleobases and the modifications of the nucleotides at the corresponding positions of the specific sequence (e.g., SEQ ID NOs: 812 to 1293).

In certain aspects, when a sense strand or an antisense strand of a dsRNA in above paragraphs is differing by a certain number of nucleotides (e.g., one, two or three nucleotides) from a specific sequence (e.g., SEQ ID NOs: 812 to 1293), it is meant by that the sense strand or the antisense strand includes one, two or three nucleotides, having different nucleobases, different modifications, and/or different phosphate linkages (e.g., phosphorothioate (PS)), compared to the nucleobases, the modifications, and/or the phosphate linkages of the nucleotides at the corresponding positions of the specific sequence (e.g., SEQ ID NOs: 812 to 1293). In some embodiments, when a sense strand or an antisense strand is differing by a certain number of nucleotides (e.g., one, two or three nucleotides) from a specific sequence (e.g., SEQ ID NOs: 812 to 1293), the sense strand or the antisense strand includes one, two, or three nucleotides, having different phosphate linkages compared to the phosphate linkages of the nucleotides at the corresponding positions of the specific sequence (e.g., SEQ ID NOs: 812 to 1293). In some embodiments, when a sense strand or an antisense strand is differing by a certain number of nucleotides (e.g., one, two or three nucleotides) from a specific sequence (e.g., SEQ ID NOs: 812 to 1293), the sense strand or the antisense strand includes one, two, or three nucleotides, having different nucleobases and different phosphate linkages compared to the nucleobases and the phosphate linkages of the nucleotides at the corresponding positions of the specific sequence (e.g., SEQ ID NOs: 812 to 1293). In some embodiments, when a sense strand or an antisense strand is differing by a certain number of nucleotides (e.g., one, two or three nucleotides) from a specific sequence (e.g., SEQ ID NOs: 812 to 1293), the sense strand or the antisense strand includes one, two, or three nucleotides, having different modifications and different phosphate linkages compared to the modifications and the phosphate linkages of the nucleotides at the corresponding positions of the specific sequence (e.g., SEQ ID NOs: 812 to 1293). In some embodiments, when a sense strand or an antisense strand is differing by a certain number of nucleotides (e.g., one, two or three nucleotides) from a specific sequence (e.g., SEQ ID NOs: 812 to 1293), the sense strand or the antisense strand includes one, two, or three nucleotides having different nucleobases, different modifications, and different phosphate linkages compared to the nucleobases, the modifications, and the phosphate linkages of the nucleotides at the corresponding positions of the specific sequence (e.g., SEQ ID NOs: 812 to 1293).

In certain aspects, the first nucleotide from the 5′ end of each strand (e.g., sense strand and antisense strand) may include an additional phosphate group or a variant thereof (e.g., phosphorothioate, phosphorodithioate, methylphosphonate, methylene phosphonate, or vinylphosphonate (VP)) attached or linked to the 5′ terminal group of the first nucleotide.

In some embodiments, the first nucleotide from the 5′ end of each strand (e.g., sense strand and antisense strand) includes a 5′-vinylphosphonate (5′-VP) group that is a chemical moiety having the structure of

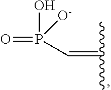 [SMILES: *=CP(=O)([O-])O]

or salts thereof, wherein [symbol] represents the point of attachment to the 5′ carbon of the pentafuranosyl sugar. In some embodiments, the first nucleotide from the 5′ end of each strand (e.g., sense strand and antisense strand) may include (E)-vinylphosphonate (VP) having a structure of

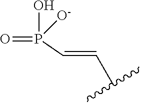

wherein [symbol] represents the point of attachment to the 4′ carbon of the pentafuranosyl sugar. In some embodiments, the first nucleotide from the 5′ end of each strand (e.g., sense strand and antisense strand) may include (Z)-vinylphosphonate having a structure of

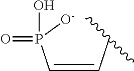

wherein [symbol] represents the point of attachment to the 4′ carbon of the pentafuranosyl sugar.

In certain aspects, one or more of the modified nucleotides contain a 2′ modification (e.g., 2′-OMe, 2′-F, 2′-MOE, 2′-deoxy, etc.) and an internucleoside linkage modification (e.g., phosphorothioate or (E)-vinylphosphonate). In some embodiments, one or more of the modified nucleotides contain 2′-OMe modification and phosphorothioate group. In some embodiments, one or more of the modified nucleotides contain 2′-OMe modification and (E)-vinylphosphonate group. In some embodiments, one or more of the modified nucleotides contain 2′-F modification and phosphorothioate group. In some embodiments, one or more of the modified nucleotides contain 2′-F and (E)-vinylphosphonate group. In some embodiments, one or more of the modified nucleotides contain 2′-MOE modification and phosphorothioate group. In some embodiments, one or more of the modified nucleotides contain 2′-MOE modification and (E)-vinylphosphonate group. In some embodiments, one or more of the modified nucleotides contain 2′-deoxy modification and phosphorothioate group. In some embodiments, one or more of the modified nucleotides contain 2′-OMe modification and (E)-vinylphosphonate group. In some embodiments, one or more of the modified nucleotides are GNA containing (E)-vinylphosphonate group. In some embodiments, one or more of the modified nucleotides are GNA containing a phosphorothioate group.

In certain aspects, the modified nucleotides contain one or more modifications on a modified nucleobase. In some embodiments, one or more of the modified nucleotides may include thymine (“T”) nucleobase (“ribothymidine” or “5-methyluridine”) in the ribonucleotide (e.g., including 2′-OH). In some embodiments, one or more of the modified nucleotides may include methylcytosine nucleobase (e.g., 5-methylcytidine or N4-methylcytidine). In certain aspects, one or more of the modified nucleotides may contain no nucleobase or be abasic.

### Sense Strand (SS)

In certain aspects, a sense strand of the dsRNA agent(s) as described herein are substantially (e.g., greater than about 80%, 85%, 90%, or 95% of the total nucleotides) made of modified nucleotides. In another certain aspect, the sense strand is entirely made of modified nucleotides.

In certain aspects, a sense strand of the dsRNA agent(s) as described herein includes two or more 2′-MOE modifications. In some embodiments, the sense strand includes two, four, six or eight 2′-MOE modifications. In some embodiments, the sense strand includes two 2′-MOE modifications. In some embodiments, the sense strand includes four 2′-MOE modifications. In some embodiments, the sense strand includes six 2′-MOE modifications. In some embodiments, the sense strand includes eight 2′-MOE modifications.

In some embodiments, the 2′-MOE modified nucleotides in the sense strand as described herein include a structure of

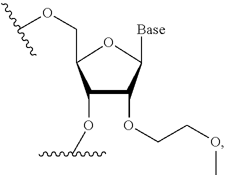

or a pharmaceutically acceptable salt thereof, wherein [symbol] is an attachment point to a linkage (e.g., phosphate or phosphorothioate group) or the adjacent nucleotides and “Base” is a nucleobase.

In some embodiments, the 2′-MOE modified nucleotides in the sense strand as described herein include a structure of

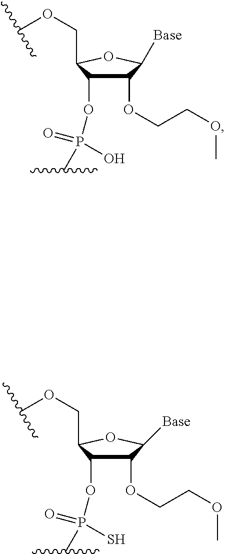

or a pharmaceutically acceptable salt thereof, wherein [symbol] is an attachment point to a terminal group (e.g., H, OH, or salt) or the adjacent nucleotides and “Base” is a nucleobase. In some embodiments, the 2′-MOE modified nucleotides in the sense strand as described herein include a structure of

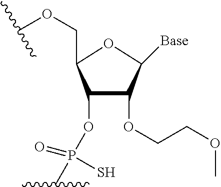

or a pharmaceutically acceptable salt thereof.

In some embodiments, the 2′-MOE modified nucleotides include a structure of

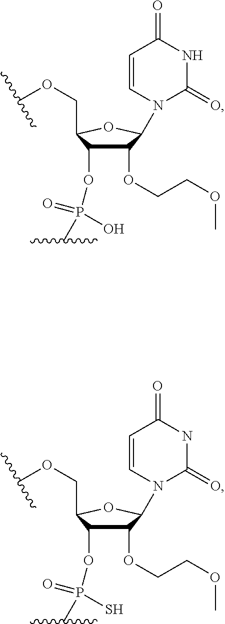

or a pharmaceutically acceptable salt thereof, wherein [symbol] is an attachment point to a terminal group (e.g., H, OH, or salt) or the adjacent nucleotides. In some embodiments, the 2′-MOE modified nucleotides include a structure of

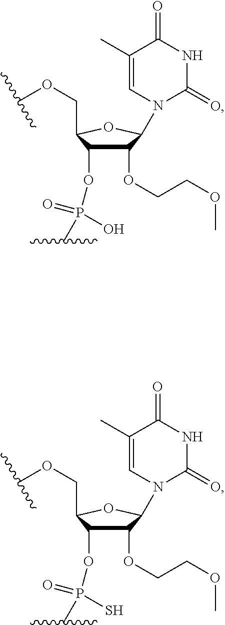

or a pharmaceutically acceptable salt thereof. In some embodiments, the 2′-MOE modified nucleotides include a structure of

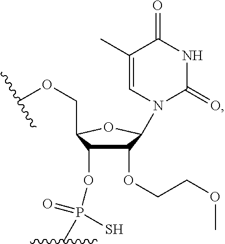

or a pharmaceutically acceptable salt thereof.

In some embodiments, the 2′-MOE modified nucleotides include a nucleotide having a structure of

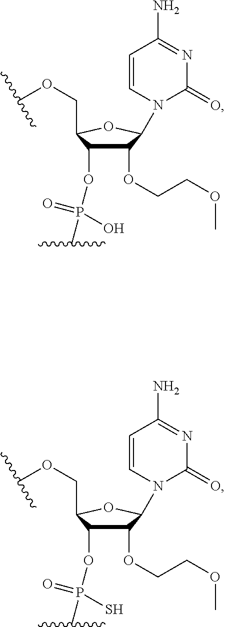

or a pharmaceutically acceptable salt thereof, wherein [symbol] is an attachment point to a terminal group (e.g., H, OH, or salt) or the adjacent nucleotides. In some embodiments, the 2′-MOE modified nucleotides include a nucleotide having a structure of

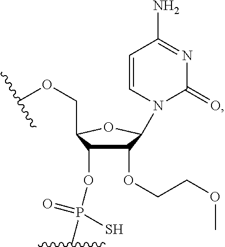

or a pharmaceutically acceptable salt thereof. In some embodiments, the 2′-MOE modified nucleotides include a nucleotide having a structure of

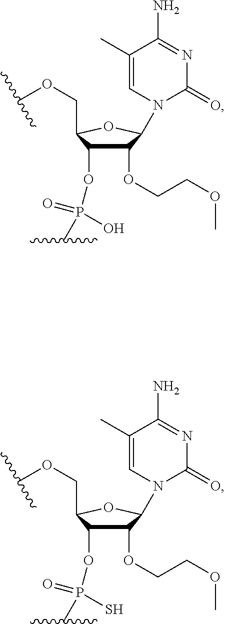

or a pharmaceutically acceptable salt thereof. In some embodiments, the 2′-MOE modified nucleotides include a nucleotide having a structure of

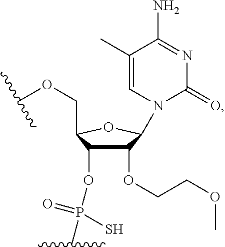

or a pharmaceutically acceptable salt thereof.

In some embodiments, the 2′-MOE modified nucleotides in the sense strand as described herein include a nucleotide having a structure of

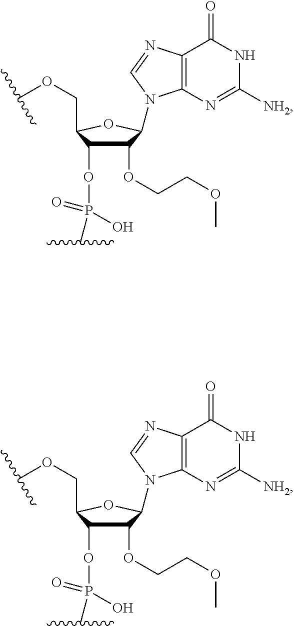

or a pharmaceutically acceptable salt thereof, wherein [symbol] is an attachment point to a terminal group (e.g., H, OH, or salt) or the adjacent nucleotides. In some embodiments, the 2′-MOE modified nucleotides in the sense strand as described herein include a nucleotide having a structure of

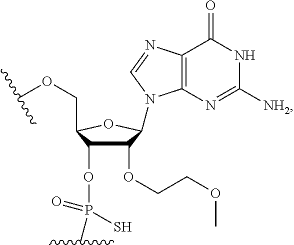

or a pharmaceutically acceptable salt thereof.

In some embodiments, the 2′-MOE modified nucleotides include a nucleotide having a structure of

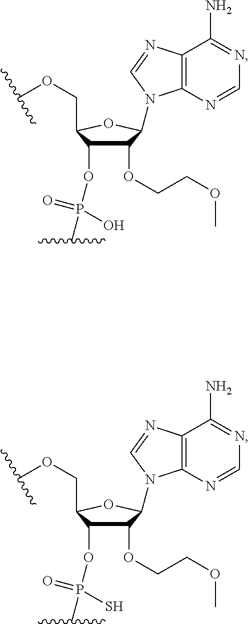

or a pharmaceutically acceptable salt thereof, wherein [symbol] is an attachment point to a terminal group (e.g., H, OH, or salt) or the adjacent nucleotides. In some embodiments, the 2′-MOE modified nucleotides include a nucleotide having a structure of

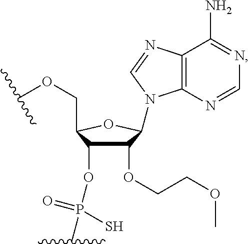

or a pharmaceutically acceptable salt thereof.

In certain aspects, at least one of the 2′-MOE modified nucleotides in the sense strand as described herein has a structure of

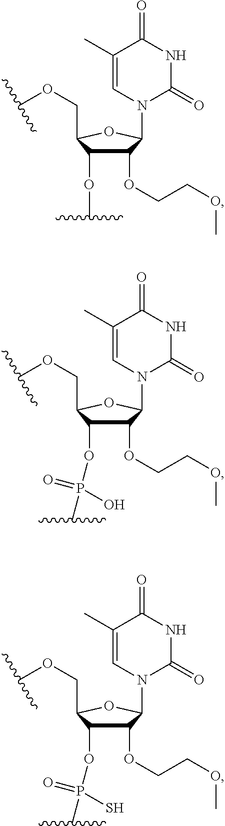

or a pharmaceutically acceptable salt thereof, wherein [symbol] is an attachment point to a terminal group (e.g., H, OH, or salt) or the adjacent nucleotides. In some embodiments, at least one of the 2′-MOE modified nucleotides in the sense strand as described herein has a structure of

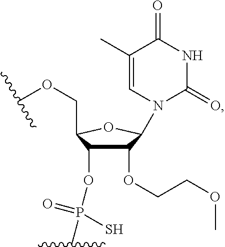

or a pharmaceutically acceptable salt thereof.

In some embodiments, the first nucleotide from the 5′ end of the sense strand includes a structure of

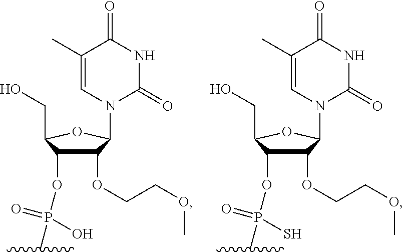

or a pharmaceutically acceptable salt thereof. In some embodiments, the first nucleotide from the 5′ end of the sense strand includes a structure of

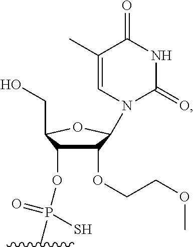

or a pharmaceutically acceptable salt thereof.

In some embodiments, the first nucleotide from the 3′ end of the sense strand includes a structure of

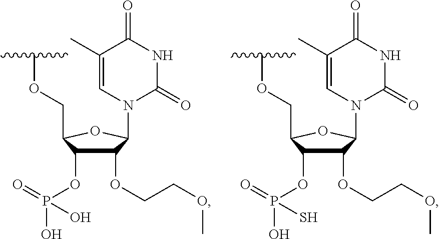

or a pharmaceutically acceptable salt thereof. In some embodiments, the first nucleotide from the 3′ end of the sense strand includes a structure of

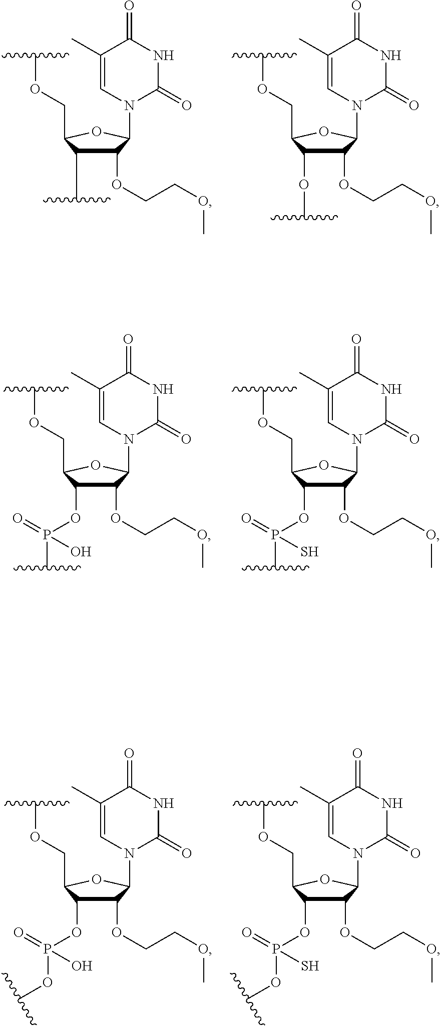

or a pharmaceutically acceptable salt thereof, wherein [symbol] is an attachment point to a ligand. In some embodiments, the first nucleotide from the 3′ end of the sense strand includes a structure of

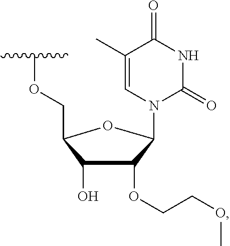

or a pharmaceutically acceptable salt thereof.

In certain aspects, at least one of the 2′-MOE modified nucleotides in the sense strand as described herein has a structure of

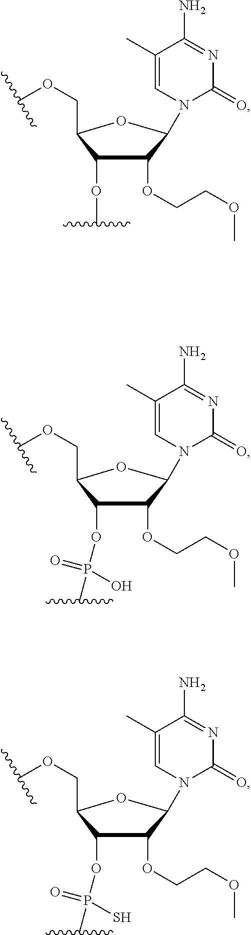

or a pharmaceutically acceptable salt thereof, wherein [symbol] is an attachment point to a terminal group (e.g., H, OH, or salt) or the adjacent nucleotides. In some embodiments, at least one of the 2′-MOE modified nucleotides in the sense strand as described herein has a structure of

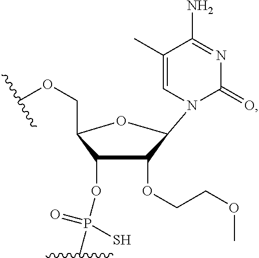

or a pharmaceutically acceptable salt thereof.

In some embodiments, the first nucleotide from the 5′ end of the sense strand includes a structure of

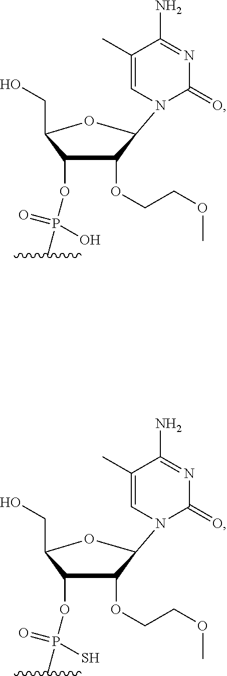

or a pharmaceutically acceptable salt thereof. In some embodiments, the first nucleotide from the 5′ end of the sense strand includes a structure of

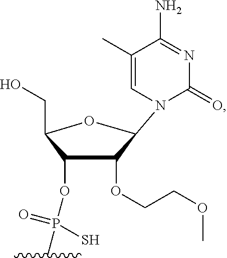

or a pharmaceutically acceptable salt thereof.

In some embodiments, the first nucleotide from the 3′ end of the sense strand includes a structure of

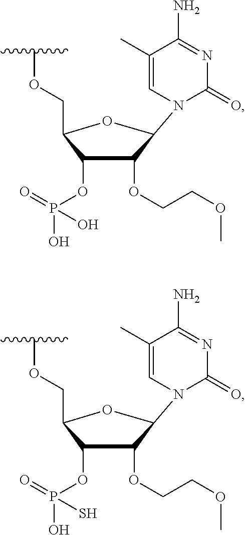

or a pharmaceutically acceptable salt thereof. In some embodiments, the first nucleotide from the 3′ end of the sense strand includes a structure of

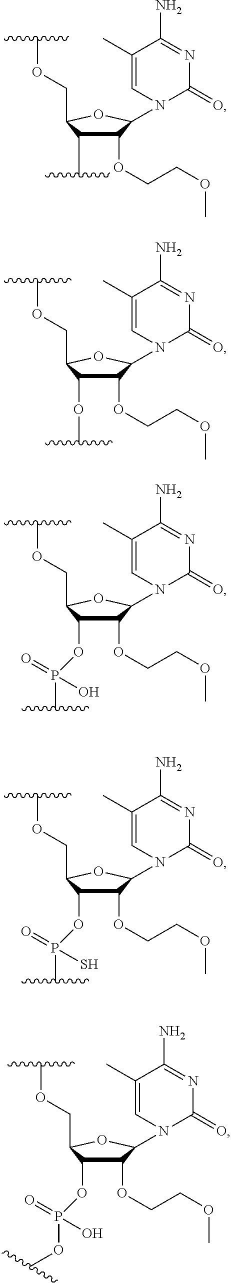

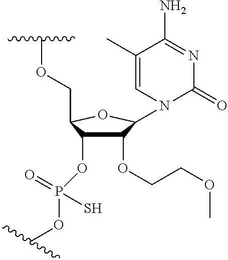

or a pharmaceutically acceptable salt thereof, wherein [symbol] is an attachment point to a ligand. In some embodiments, the first nucleotide from the 3′ end of the sense strand includes a structure of

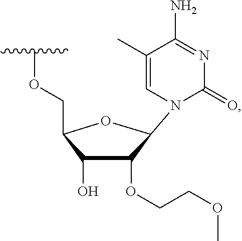

or a pharmaceutically acceptable salt thereof.

In certain aspects, at least one of the 2′-MOE modified nucleotides in the sense strand as described herein has a structure of

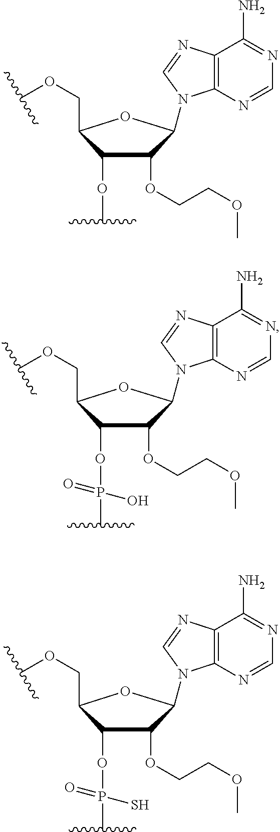

or a pharmaceutically acceptable salt thereof, wherein [symbol] is an attachment point to a terminal group (e.g., H, OH, or salt) or the adjacent nucleotides. In some embodiments, at least one of the 2′-MOE modified nucleotides in the sense strand as described herein has a structure of

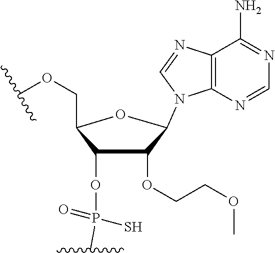

or a pharmaceutically acceptable salt thereof.

In some embodiments, the first nucleotide from the 5′ end of the sense strand includes a structure of

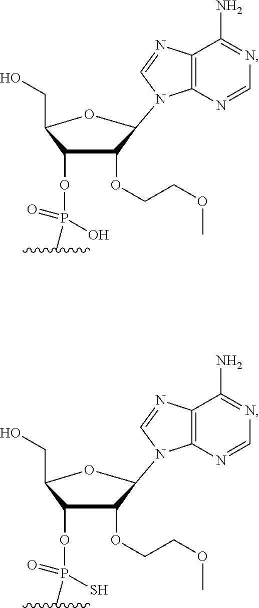

or a pharmaceutically acceptable salt thereof. In some embodiments, the first nucleotide from the 5′ end of the sense strand includes a structure of

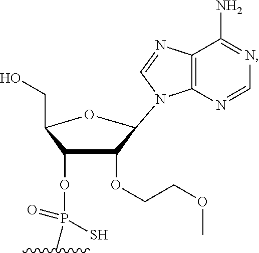

or a pharmaceutically acceptable salt thereof.

In some embodiments, the first nucleotide from the 3′ end of the sense strand has a structure of

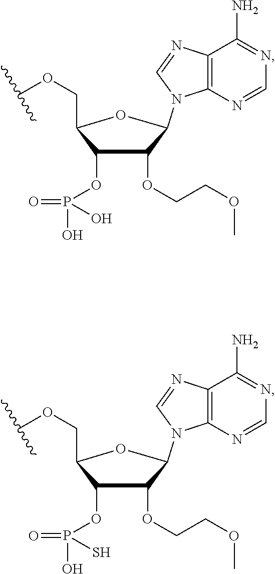

or a pharmaceutically acceptable salt thereof. In some embodiments, the first nucleotide from the 3′ end of the sense strand has a structure of

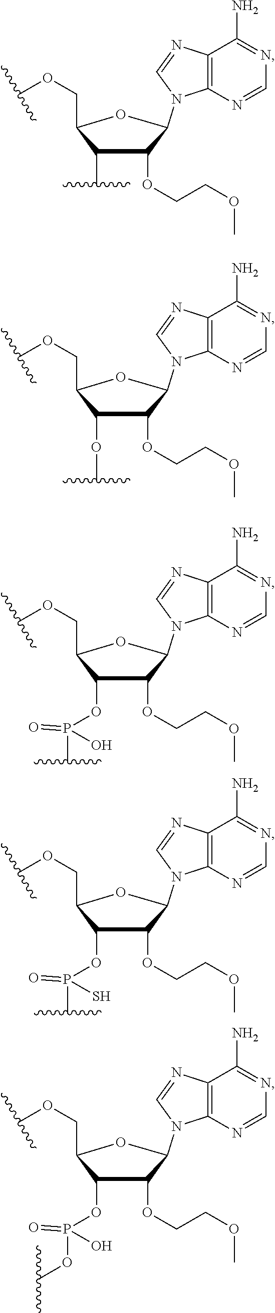

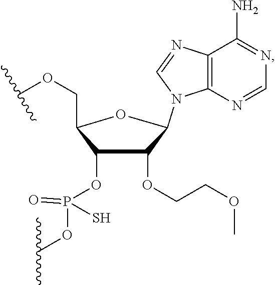

or a pharmaceutically acceptable salt thereof, wherein [symbol] is an attachment point to a ligand. In some embodiments, the first nucleotide from the 3′ end of the sense strand has a structure of

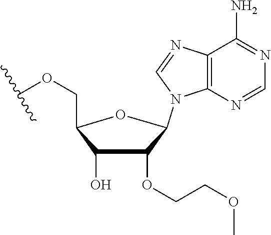

or a pharmaceutically acceptable salt thereof.

In certain aspects, at least one of the 2′-MOE modified nucleotides in the sense strand as described herein has a structure of

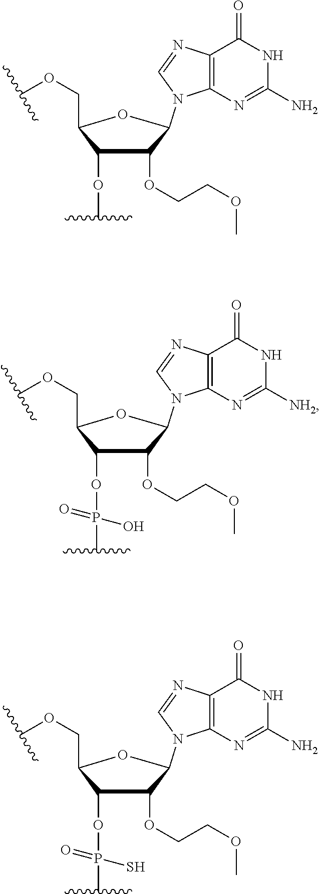

or a pharmaceutically acceptable salt thereof, wherein [symbol] is an attachment point to a terminal group (e.g., H, OH, or salt) or the adjacent nucleotides. In some embodiments, at least one of the 2′-MOE modified nucleotides in the sense strand as described herein has a structure of

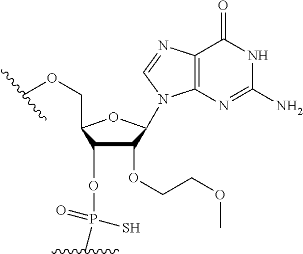

or a pharmaceutically acceptable salt thereof.

In some embodiments, the first nucleotide from the 5′ end of the sense strand includes a structure of

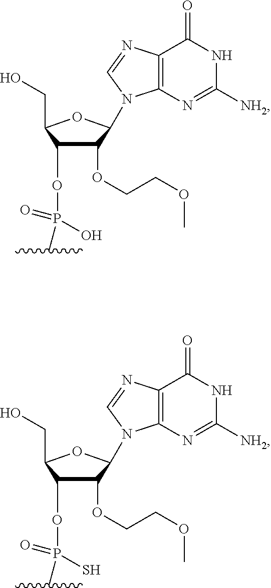

or a pharmaceutically acceptable salt thereof. In some embodiments, the first nucleotide from the 5′ end of the sense strand includes a structure of

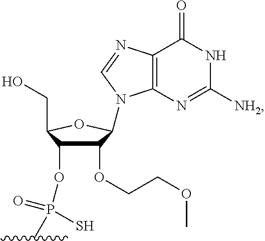

or a pharmaceutically acceptable salt thereof.

In some embodiments, the first nucleotide from the 3′ end of the sense strand has a structure of

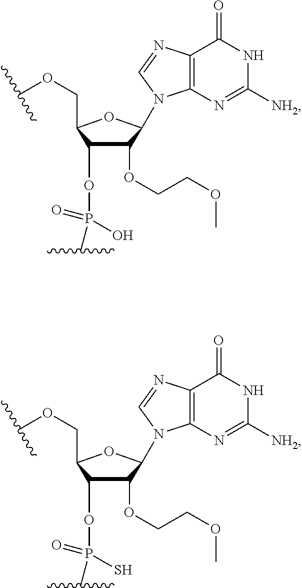

or a pharmaceutically acceptable salt thereof. In some embodiments, the first nucleotide from the 3′ end of the sense strand has a structure of

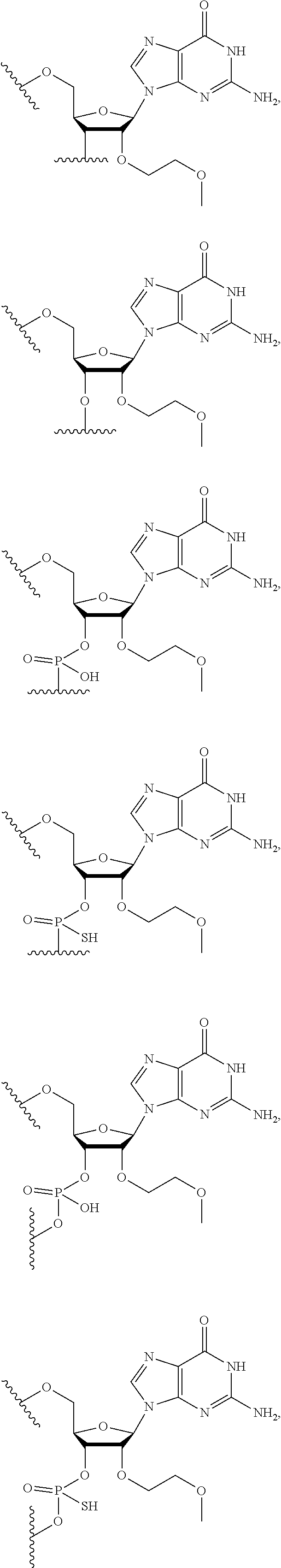

or a pharmaceutically acceptable salt thereof, wherein [symbol] is an attachment point to a ligand. In some embodiments, the first nucleotide from the 3′ end of the sense strand has a structure of

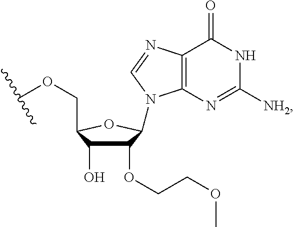

or a pharmaceutically acceptable salt thereof.

In certain aspects, the 2′-MOE modified nucleotides locate at both 5′ and 3′ ends of a sense strand so as to form a structural confinement (“2′-MOE clamp”) at the sense strand termini. In some embodiments, the 2′-MOE clamps may be symmetric and having the same number of 2′-MOE modified nucleotides at both 5′ and 3′ ends of the sense strand. For example, the sense strand includes one 2′-MOE modified nucleotide at 5′ end and one 2′-MOE modified nucleotide at 3′ end; two 2′-MOE modified nucleotides at 5′ end and two 2′-MOE modified nucleotides at 3′ end; or three 2′-MOE modified nucleotides at 5′ end and three 2′-MOE modified nucleotides at 3′ end. In some embodiments, the 2′-MOE clamps may be asymmetric and having different numbers of 2′-MOE nucleotides at 5′ and 3′ ends of the sense strand. For example, the sense strand includes one 2′-MOE modified nucleotide at 5′ end only; one 2′-MOE modified nucleotide at 3′ end only; two 2′-MOE modified nucleotides at 5′ end only; two 2′-MOE modified nucleotides at 3′ end only; one 2′-MOE modified nucleotide at 5′ end and two 2′-MOE modified nucleotides at 3′ end; or two 2′-MOE modified nucleotides at 5′ end and one 2′-MOE modified nucleotide at 3′ end.

In certain aspects, the sense strand includes one 2′-MOE modified nucleotide at 5′ end and one 2′-MOE modified nucleotide at 3′ end. In some embodiments, the sense strand includes only one 2′-MOE modified nucleotide at 5′ end and only one 2′-MOE modified nucleotide at 3′ end. In some embodiments, the sense strand includes only one 2′-MOE modified nucleotide at 5′ end. In some embodiments, the sense strand includes only one 2′-MOE modified nucleotide at 3′ end.

In certain aspects, the sense strand includes at least two contiguous 2′-MOE modified nucleotides at 5′ end and at least two 2′-MOE modified nucleotides at 3′ end. In some embodiments, the sense strand includes only two 2′-MOE modified nucleotides at 5′ end and only two 2′-MOE modified nucleotides at 3′ end. In some embodiments, the sense strand includes only two 2′-MOE modified nucleotides at 5′ end. In some embodiments, the sense strand includes only two 2′-MOE modified nucleotides at 3′ end.

In certain aspects, the sense strand is 21 nucleotides in length. In some embodiments, the sense strand includes one, two, three, or four 2′-MOE modified nucleotides positioned at the 1st, 2nd, 20th, and/or 21^{st} nucleotides from the 5′ end of the sense strand. In some embodiments, the sense strand includes two 2′-MOE modified nucleotides positioned at the 1st, 2nd, 20th, or 21^{st} nucleotides from the 5′ end of the sense strand. In some embodiments, the sense strand includes three 2′-MOE modified nucleotides positioned at the 1st, 2nd, 20th, or 21st nucleotides from the 5′ end of the sense strand. In some embodiments, the sense strand includes 2′-MOE modified nucleotides positioned at the 1st, 2nd, 20th, and 21^{st} nucleotides from the 5′ end of the sense strand. In some embodiments, the sense strand does not include a 2′-MOE modified nucleotide at the 3rd to 19th nucleotides from 5′ end of the sense strands.

Alternatively, in certain aspects, a sense strand of the dsRNA as described herein includes two or more TNAs. In some embodiments, the sense strand includes two, four, six or eight TNAs. In some embodiments, the sense strand includes two TNAs. In some embodiments, the sense strand includes four TNAs. In some embodiments, the sense strand includes six TNAs. In some embodiments, the sense strand includes eight TNAs.

In certain aspects, the sense strand includes at least two contiguous TNAs at 5′ end and at least two TNAs at 3′ end. In some embodiments, the sense strand includes only two TNAs at 5′ end and only two TNAs at 3′ end. In some embodiments, the sense strand includes only two TNAs at 5′ end. In some embodiments, the sense strand includes only two TNAs at 3′ end.

In some embodiments, the TNAs in the sense strand as described herein include a structure of

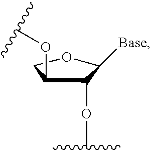

or a pharmaceutically acceptable salt thereof, wherein [symbol] is an attachment point to a linkage (e.g., phosphate or phosphorothioate group) or the adjacent nucleotides and “Base” is a nucleobase.

In some embodiments, the TNAs in the sense strand as described herein include a structure of

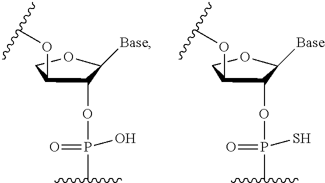

or a pharmaceutically acceptable salt thereof wherein [symbol] is an attachment point to a terminal group (e.g., H, OH, or salt) or the adjacent nucleotides and “Base” is a nucleobase. In some embodiments, the TNAs in the dsRNA as described herein include a structure of

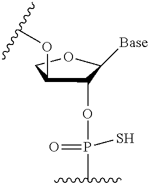

or a pharmaceutically acceptable salt thereof.

In some embodiments, the TNAs include a structure of

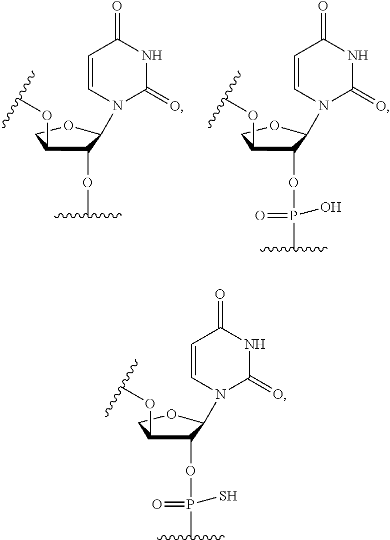

or a pharmaceutically acceptable salt thereof, wherein [symbol] is an attachment point to a terminal group (e.g., H, OH, or salt) or the adjacent nucleotides. In some embodiments, the TNAs include a structure of

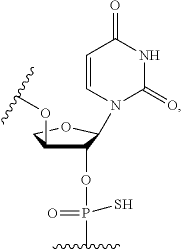

or a pharmaceutically acceptable salt thereof.

In some embodiments, the TNAs include a nucleotide having a structure of

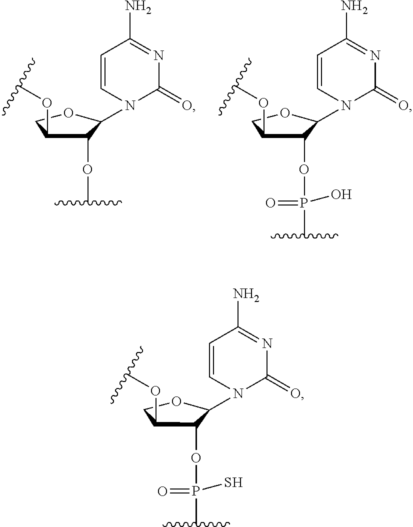

or a pharmaceutically acceptable salt thereof, wherein [symbol] is an attachment point to a terminal group (e.g., H, OH, or salt) or the adjacent nucleotides. In some embodiments, the TNAs include a nucleotide having a structure of

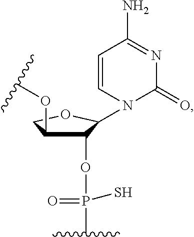

or a pharmaceutically acceptable salt thereof

In some embodiments, the TNAs in the dsRNA as described herein include a nucleotide having a structure of

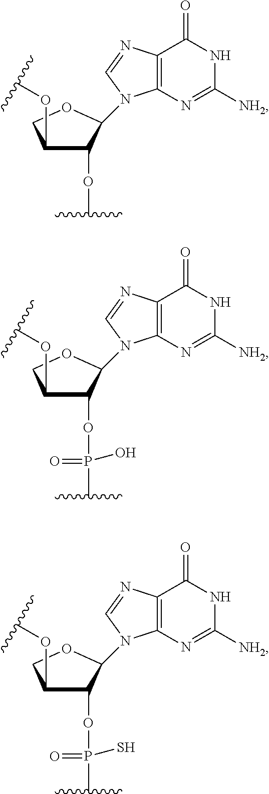

or a pharmaceutically acceptable salt thereof, wherein [symbol] is an attachment point to a terminal group (e.g., H, OH, or salt) or the adjacent nucleotides. In some embodiments, the TNAs in the dsRNA as described herein include a nucleotide having a structure of

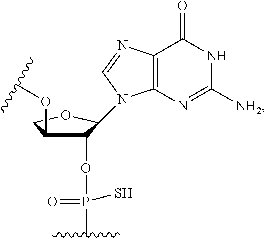

or a pharmaceutically acceptable salt thereof.

In some embodiments, the TNAs include a nucleotide having a structure of

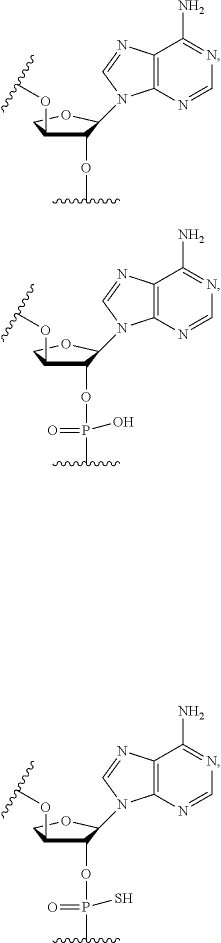

or a pharmaceutically acceptable salt thereof, wherein [symbol] is an attachment point to a terminal group (e.g., H, OH, or salt) or the adjacent nucleotides. In some embodiments, the TNAs include a nucleotide having a structure of

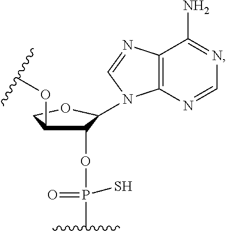

or a pharmaceutically acceptable salt thereof.

In certain aspects, at least one of the TNAs in the sense strand as described herein has a structure of

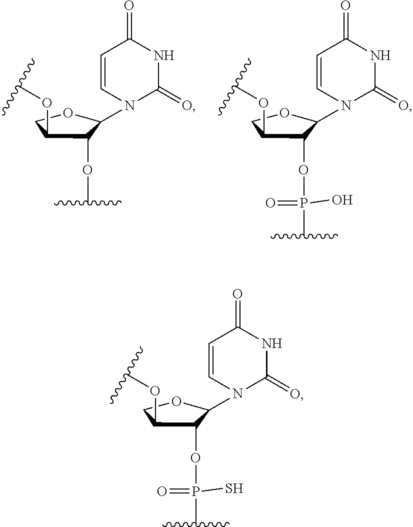

or a pharmaceutically acceptable salt thereof, wherein [symbol] is an attachment point to a terminal group (e.g., H, OH, or salt) or the adjacent nucleotides. In some embodiments, at least one of the TNAs in the sense strand as described herein has a structure of

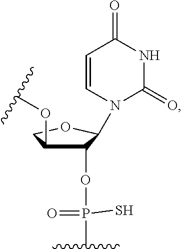

or a pharmaceutically acceptable salt thereof.

In some embodiments, the first nucleotide from the 5′ end of the sense strand includes a structure of

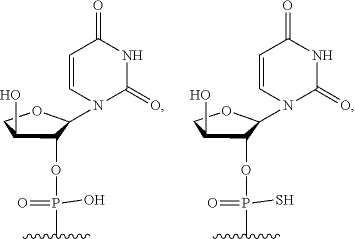

or a pharmaceutically acceptable salt thereof. In some embodiments, the first nucleotide from the 5′ end of the sense strand includes a structure of

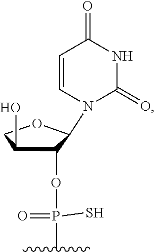

or a pharmaceutically acceptable salt thereof.

In some embodiments, the first nucleotide from the 3′ end of the sense strand includes a structure of

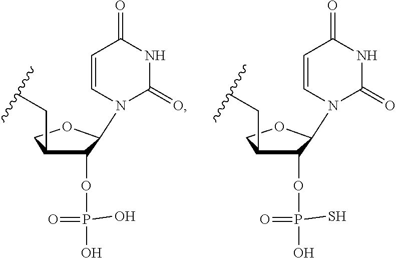

or a pharmaceutically acceptable salt thereof. In some embodiments, the first nucleotide from the 3′ end of the sense strand includes a structure of,

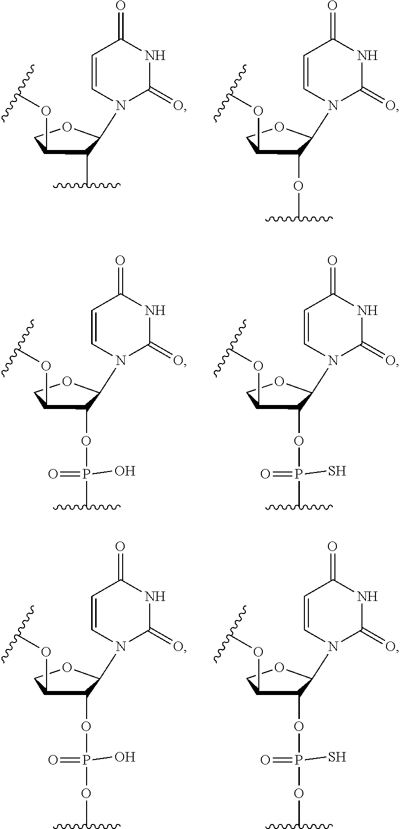

or a pharmaceutically acceptable salt thereof and [symbol] is an attachment point to a ligand. In some embodiments, the first nucleotide from the 3′ end of the sense strand includes a structure of

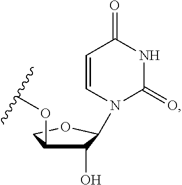

or a pharmaceutically acceptable salt thereof.

In certain aspects, at least one of the TNAs in the sense strand as described herein has a structure of

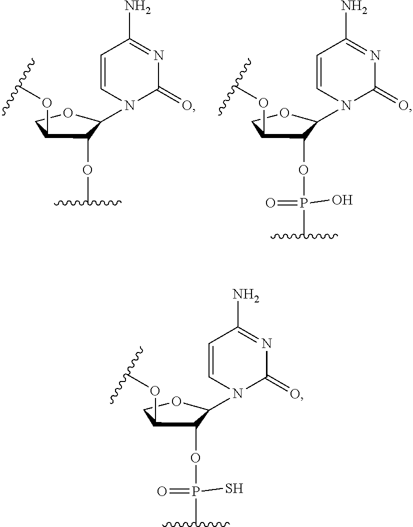

or a pharmaceutically acceptable salt thereof, wherein [symbol] is an attachment point to a terminal group (e.g., H, OH, or salt) or the adjacent nucleotides. In some embodiments, at least one of the TNAs in the sense strand as described herein has a structure of

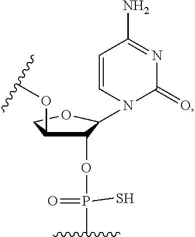

or a pharmaceutically acceptable salt thereof.

In some embodiments, the first nucleotide from the 5′ end of the sense strand includes a structure of

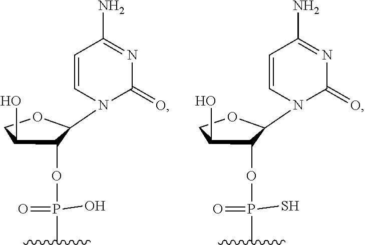

or a pharmaceutically acceptable salt thereof. In some embodiments, the first nucleotide from the 5′ end of the sense strand includes a structure of

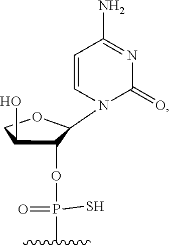

or a pharmaceutically acceptable salt thereof.

In some embodiments, the first nucleotide from the 3′ end of the sense strand includes a structure of

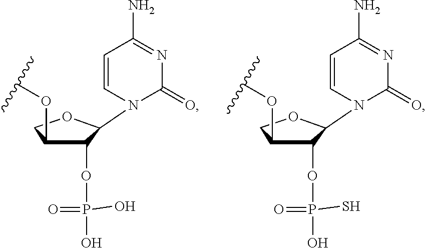

or a pharmaceutically acceptable salt thereof. In some embodiments, the first nucleotide from the 3′ end of the sense strand includes a structure of

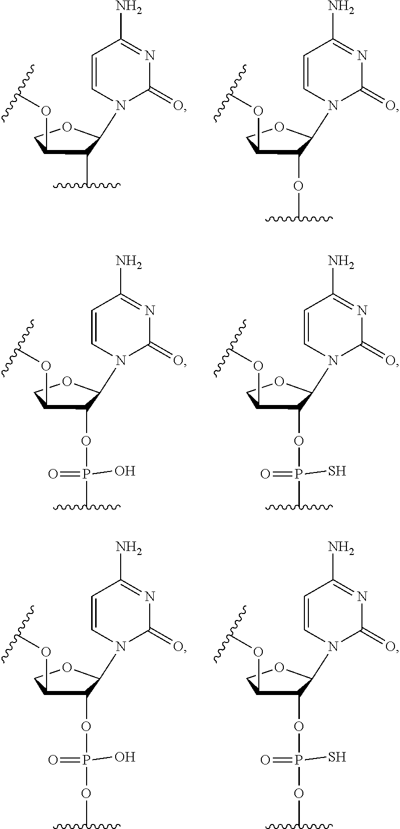

or a pharmaceutically acceptable salt thereof and [symbol] is an attachment point to a ligand. In some embodiments, the first nucleotide from the 3′ end of the sense strand includes a structure of

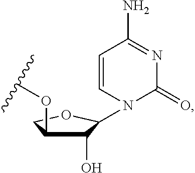

or a pharmaceutically acceptable salt thereof.

In certain aspects, at least one of the TNAs in the sense strand as described herein has a structure of

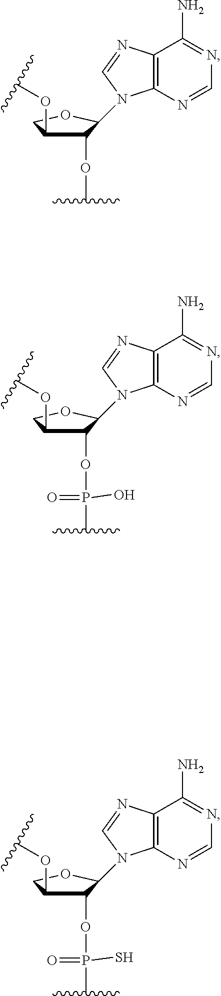

or a pharmaceutically acceptable salt thereof, wherein [symbol] is an attachment point to a terminal group (e.g., H, OH, or salt) or the adjacent nucleotides. In some embodiments, at least one of the TNAs in the sense strand as described herein has a structure of

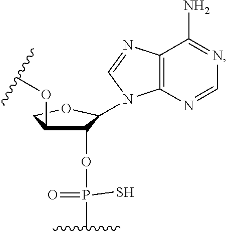

or a pharmaceutically acceptable salt thereof.

In some embodiments, the first nucleotide from the 5′ end of the sense strand includes a structure of

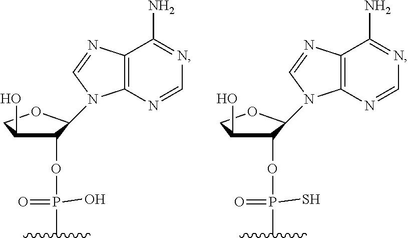

or a pharmaceutically acceptable salt thereof. In some embodiments, the first nucleotide from the 5′ end of the sense strand includes a structure of

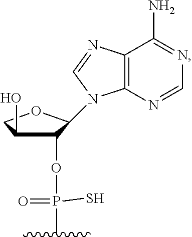

or a pharmaceutically acceptable salt thereof.

In some embodiments, the first nucleotide from the 3′ end of the sense strand includes a structure of

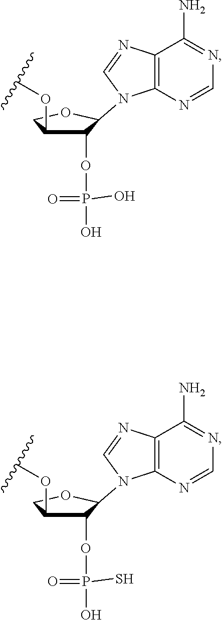

or a pharmaceutically acceptable salt thereof. In some embodiments, the first nucleotide from the 3′ end of the sense strand includes a structure of

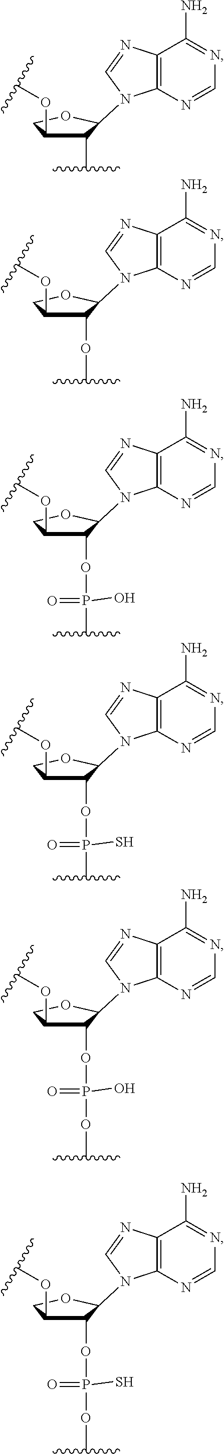

or a pharmaceutically acceptable salt thereof and [symbol] is an attachment point to a ligand. In some embodiments, the first nucleotide from the 3′ end of the sense strand includes a structure of

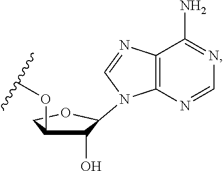

or a pharmaceutically acceptable salt thereof.

In certain aspects, at least one of the TNAs in the sense strand as described herein has a structure of

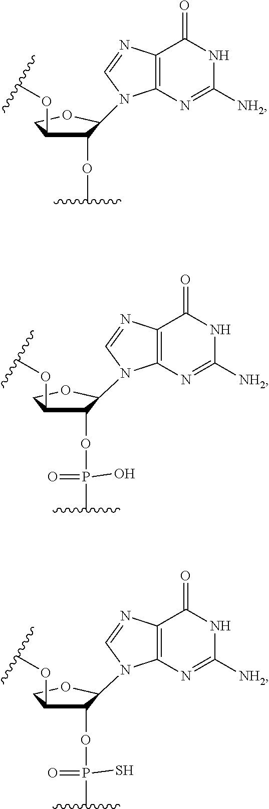

or a pharmaceutically acceptable salt thereof, wherein [symbol] is an attachment point to a terminal group (e.g., H, OH, or salt) or the adjacent nucleotides. In some embodiments, at least one of the TNAs in the sense strand as described herein has a structure of

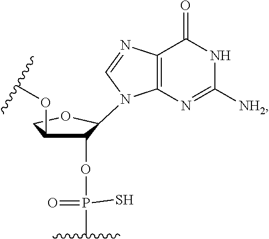

or a pharmaceutically acceptable salt thereof.

In some embodiments, the first nucleotide from the 5′ end of the sense strand includes a structure of

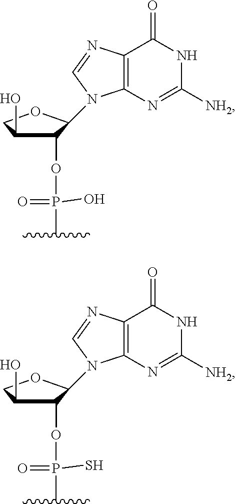

or a pharmaceutically acceptable salt thereof. In some embodiments, the first nucleotide from the 5′ end of the sense strand includes a structure of

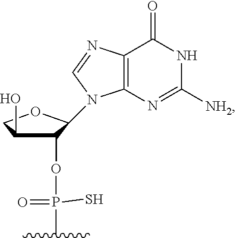

or a pharmaceutically acceptable salt thereof.

In some embodiments, the first nucleotide from the 3′ end of the sense strand includes a structure of

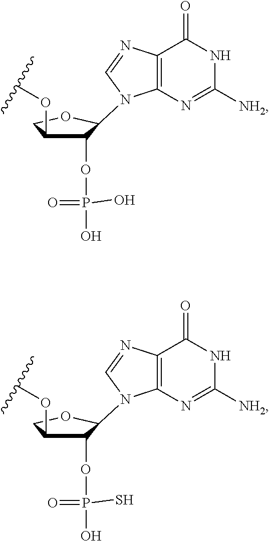

or a pharmaceutically acceptable salt thereof. In some embodiments, the first nucleotide from the 3′ end of the sense strand includes a structure of

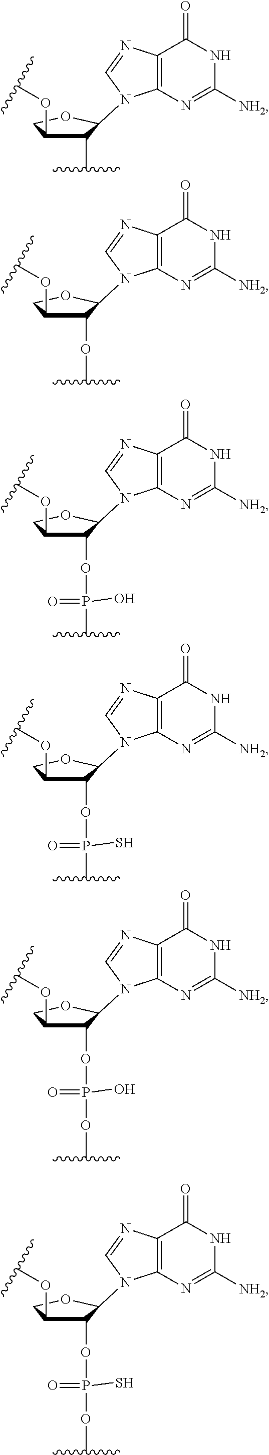

or a pharmaceutically acceptable salt thereof and [symbol] is an attachment point to a ligand. In some embodiments, the first nucleotide from the 3′ end of the sense strand includes a structure of

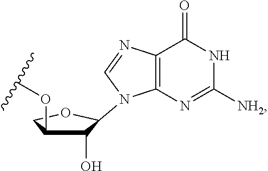

or a pharmaceutically acceptable salt thereof.

In certain aspects, the TNAs locate at both 5′ and 3′ ends of a sense strand so as to form a structural confinement (“TNA clamp”) at the sense strand termini. In some embodiments, the TNA clamps may be symmetric and having the same number of TNAs at both 5′ and 3′ ends of the sense strand. For example, the sense strand includes one TNA at 5′ end and one TNA at 3′ end; two TNAs at 5′ end and two TNAs at 3′ end; or three TNAs at 5′ end and three TNAs at 3′ end. In some embodiments, the TNA clamps may be asymmetric and having different numbers of TNAs at 5′ and 3′ ends of the sense strand. For example, the sense strand includes one TNA at 5′ end only; one TNA at 3′ end only; two TNAs at 5′ end only; two TNAs at 3′ end only; one TNA at 5′ end and two TNAs at 3′ end; or two TNAs at 5′ end and one TNA at 3′ end.

In certain aspects, the sense strand includes one TNA at 5′ end and one TNA at 3′ end. In some embodiments, the sense strand includes only one TNA at 5′ end and only one TNA at 3′ end. In some embodiments, the sense strand includes only one TNA at 5′ end. In some embodiments, the sense strand includes only one TNA at 3′ end.

In certain aspects, the sense strand includes at least two contiguous TNAs at 5′ end and at least two TNAs at 3′ end. In some embodiments, the sense strand includes only two TNAs at 5′ end and only two TNAs at 3′ end. In some embodiments, the sense strand includes only two TNAs at 5′ end. In some embodiments, the sense strand includes only two TNAs at 3′ end.

In certain aspects, the sense strand is 21 nucleotides in length. In some embodiments, the sense strand includes one, two, three, or four TNAs positioned at the 1st, 2nd, 20th, and/or 21st nucleotides from the 5′ end of the sense strand. In some embodiments, the sense strand includes two TNAs positioned at the 1st, 2nd, 20th, or 21st nucleotides from the 5′ end of the sense strand. In some embodiments, the sense strand includes three TNAs positioned at the 1st, 2nd, 20th, or 21st nucleotides from the 5′ end of the sense strand. In some embodiments, the sense strand includes TNAs positioned at the 1st, 2nd, 20th, and 21st nucleotides from the 5′ end of the sense strand.

In certain aspects, the sense strand of the dsRNA as described herein includes two or more 2′-F modifications. In some embodiments, the sense strand of the dsRNA includes two, three, four, five, six, seven, or eight 2′-F modified nucleotides. In some embodiments, the sense strand includes two 2′-F modified nucleotides. In some embodiments, the sense strand includes three 2′-F modified nucleotides. In some embodiments, the sense strand includes four 2′-F modified nucleotides. In some embodiments, the sense strand includes five 2′-F modified nucleotides. In some embodiments, the sense strand includes six 2′-F modified nucleotides. In some embodiments, the sense strand includes seven 2′-F modified nucleotides. In some embodiments, the sense strand includes eight 2′-F modified nucleotides. In some embodiments, two contiguous 2′-F modified nucleotides locate in the sense strand. In some embodiments, three contiguous 2′-F modified nucleotides locate in the sense strand. In some embodiments, four contiguous 2′-F modified nucleotides locate in the sense strand.

In certain aspects, the sense strand is 21 nucleotides in length. In some embodiments, 2′-F modified nucleotides locate at 5th, 7th, 8th, and/or 9th positions from the 5′ end of the sense strand. In some embodiments, 2′-F modified nucleotides locate at 6th, 8th, 9th, and/or 10th positions from the 5′ end of the sense strand. In some embodiments, 2′-F modified nucleotides locate at 7th, 9th, 10th, and/or 11th positions from the 5′ end of the sense strand. In some embodiments, 2′-F modified nucleotides locate at 8th, 10th, 11th, and/or 12th positions from the 5′ end of the sense strand. In some embodiments, 2′-F modified nucleotides locate at 9th, 11th, 12th, and/or 13th positions from the 5′ end of the sense strand.

In some embodiments, the sense strand includes 2′-OMe modified nucleotides in the remaining positions in the sense strand.

In certain aspects, the sense strand includes one to six (e.g., 1, 2, 3, 4, 5 or 6) phosphorothioate (PS) linkages between nucleosides. In some embodiments, the sense strand includes one, two, three, or four phosphorothioate (PS) linkages between nucleosides.

In certain aspects, the sense strand is 21 nucleotides in length. In some embodiments, the sense strand includes two 3′-PS modified nucleotides at the 1st, 2nd, 19th and/or 20th positions from 5′-end of the sense strand. In some embodiments, the sense strand includes three 3′-PS modified nucleotides at the 1st, 2nd, 19th and/or 20th positions from 5′-end of the sense strand. In some embodiments, the sense strand includes 3′-PS modified nucleotides at the 1st, 2nd, 19th and 20th positions from 5′-end of the sense strand.

In certain aspects, the sense strand is 21 nucleotides in length. In some embodiments, the sense strand includes 3′-PS modified nucleotides at the 1st and 2nd positions from 5′-end of the sense strand. In some embodiments, the sense strand includes a 3′-PS modified nucleotide at the 1st position from 5′-end of the sense strand. In some embodiments, the sense strand includes 3′-PS modified nucleotides at the 1st and 20th positions from 5′-end of the sense strand.

In certain aspects, the sense strand includes two to eight phosphorothioate (PS) groups or linkages between nucleosides. In certain aspects, the sense strand is 21 nucleotides in length. In some embodiments, the sense strand includes two 3′-PS modified nucleotides positioned at the 1st, 2nd, 3rd, 4th, 17th, 18th, 19th and/or 20th nucleotides from 5′-end of the sense strand. In some embodiments, the sense strand includes four 3′-PS modified nucleotides positioned at the 1st, 2nd, 3rd, 4th, 17th, 18th, 19th and/or 20th nucleotides from 5′-end of the sense strand. In some embodiments, the sense strand includes six 3′-PS modified nucleotides positioned at the 1st, 2nd, 3rd, 4th, 17th, 18th, 19th and/or 20th nucleotides from 5′-end of the sense strand. In some embodiments, the sense strand includes 3′-PS modified nucleotides positioned at the 1st, 2nd, 3rd, 4th, 17th, 18th, 19th and 20th nucleotides from 5′-end of the sense strand.

In certain aspects, the sense strand is 21 nucleotides in length. In some embodiments, at least one of the 3′-PS groups at the 1st, 2nd, 3rd, 4th, 17th, 18th, 19th and/or 20th nucleotides from 5′-end of the sense strand is a stereopure Rp isomer. In some embodiments, at least one of the 3′-PS groups at the 1st, 2nd, 19th and/or 20th nucleotides from 5′-end of the sense strand is a stereopure Rp isomer. In some embodiments, at least one of the 3′-PS groups at the 1st and/or 20th nucleotides from 5′-end of the sense strand is a stereopure Rp isomer. In some embodiments, the 3′-PS group at the 1^{st} nucleotide from 5′-end of the sense strand is a stereopure Rp isomer. In some embodiments, the 3′-PS group at the 2nd nucleotide from 5′-end of the sense strand is a stereopure Rp isomer. In some embodiments, the 3′-PS group at the 19th nucleotide from 5′-end of the sense strand is a stereopure Rp isomer. In some embodiments, the 3′-PS group at the 20th nucleotide from 5′-end of the sense strand is a stereopure Rp isomer. In some embodiments, the 3′-PS groups at the 1st and 20th nucleotides from 5′-end of the sense strand are stereopure Rp isomers. In some embodiments, the 3′-PS groups at the 1st, 2nd, 19^{th} and 20th nucleotides from 5′-end of the sense strand are stereopure Rp isomers.

In certain aspects, the sense strand is 21 nucleotides in length. In some embodiments, at least one of the 3′-PS groups at the 1st, 2nd, 3rd, 4th, 17th, 18th, 19th and/or 20th nucleotides from 5′-end of the sense strand is a stereopure Sp isomer. In some embodiments, at least one of the 3′-PS groups at the 1st, 2nd, 19th and/or 20th nucleotides from 5′-end of the sense strand is a stereopure Sp isomer. In some embodiments, at least one of the 3′-PS groups at the 1st and/or 20th nucleotides from 5′-end of the sense strand is a stereopure Sp isomer. In some embodiments, the 3′-PS group at the 1st nucleotide from 5′-end of the sense strand is a stereopure Sp isomer. In some embodiments, the 3′-PS group at the 2nd nucleotide from 5′-end of the sense strand is a stereopure Sp isomer. In some embodiments, the 3′-PS group at the 19th nucleotide from 5′-end of the sense strand is a stereopure Sp isomer. In some embodiments, the 3′-PS group at the 20nd nucleotide from 5′-end of the sense strand is a stereopure Sp isomer. In some embodiments, the 3′-PS groups at the 1st and 20th nucleotides from 5′-end of the sense strand are stereopure Sp isomers. In some embodiments, the 3′-PS groups at the 1st, 2nd, 19th, and 20th nucleotides from 5′-end of the sense strand are stereopure Sp isomers.

In certain aspects, a sense strand of the dsRNA as described herein includes one or more of 2′-MOE modified nucleotides, one or more of 2′-F modified nucleotides, and one or more of 2′-OMe modified nucleotides. In certain aspects, a sense strand of the dsRNA as described herein consists of 2′-MOE modified nucleotides, 2′-F modified nucleotides and 2′-OMe modified nucleotides.

In certain aspects, a sense strand of the dsRNA as described herein may have a Formula (I),

5′-X₁-X₂-X₃-X₄-X₅-X₆-X₇-X₈-X₉-X₁₀-X₁₁-X₁₂-X₁₃-X₁₄-
X₁₅-X₁₆-X₁₇-X₁₈-X₁₉-X₂₀-X₂₁-3′ (I)

wherein:
each X₁ to X₂₁ is independently a nucleotide,
each X₁, X₂, X₂₀, and X₂₁ is a 2′-MOE modified nucleotide;
each X₃ to X₁₉ is independently selected from a deoxyribonucleotide, 2′-MOE modified nucleotide, 2′-F modified nucleotide, and 2′-OMe modified nucleotide.

In some embodiments, the first nucleotide from the 5′ end of the sense strand (X₁) is a 2′-MOE modified nucleotide with a nucleobase T. In some embodiments, the second nucleotide from the 5′ end of the sense strand (X₂) is a 2′-MOE modified nucleotide with a nucleobase T, G, or methylated cytosine (e.g., 5-methylcytosine or N⁴-methylcytosine). In some embodiments, the second nucleotide from the 5′ end of the sense strand (X₂) is a 2′-MOE modified nucleotide with a nucleobase T. In some embodiments, the second nucleotide from the 5′ end of the sense strand (X₂) is a 2′-MOE modified nucleotide with a nucleobase G. In some embodiments, the second nucleotide from the 5′ end of the sense strand (X₂) is a 2′-MOE modified nucleotide with a nucleobase methylated cytosine (e.g., 5-methylcytosine or N⁴-methylcytosine).

In some embodiments, the first nucleotide from the 3′ end of the sense strand (X₂₁) is a 2′-MOE modified nucleotide with a nucleobase A or T. In some embodiments, the first nucleotide from the 3′ end of the sense strand (X₂₁) is a 2′-MOE modified nucleotide with a nucleobase A. In some embodiments, the first nucleotide from the 3′ end of the sense strand (X₂₁) is a 2′-MOE modified nucleotide with a nucleobase T. In some embodiments, the second nucleotide from the 3′ end of the sense strand (X₂₀) is a 2′-MOE modified nucleotide with a nucleobase A.

In certain aspects, X₃ to X₁₉ do not include a 2′-MOE modified nucleotide. In some embodiments, each X₃ to X₁₉ is independently selected from 2′-F modified nucleotides and 2′-OMe modified nucleotides.

In some embodiments, at least one of X₃ to X₁₉ is not a deoxyribonucleotide. In some embodiments, X₃ to X₁₉ does not include a deoxyribonucleotide.

In some embodiments, each X_{f}, X_{f+2}, X_{f+3}, and X_{f+4} is 2′-F modified nucleotide when f is an integer from 3 to 17. In some embodiments, f is 5. In some embodiments, f is 6. In some embodiments, f is 7. In some embodiments, f is 8. In some embodiments, f is 9. In some embodiments, X₅, X₇, X₈, and X₉ are 2′-F modified nucleotides. In some embodiments, X₆, X₈, X₉, and X₁₀ are 2′-F modified nucleotides. In some embodiments, X₇, X₉, X₁₀, and X₁₁ are 2′-F modified nucleotides. In some embodiments, X₈, X₁₀, X₁₁, and X₁₂ are 2′-F modified nucleotides. In some embodiments, X₉, X₁₁, X₁₂, and X₁₃ are 2′-F modified nucleotides.

In some embodiments, the sense strand includes 2′-OMe modified nucleotides in the remaining positions in the sense strand.

In some embodiments, at least two nucleotides from X₁, X₂, X₁₉, and X₂₀ contain a 3′-PS group, respectively. In some embodiments, two nucleotides from X₁, X₂, X₁₉, and X₂₀ contain a 3′-PS group, respectively. In some embodiments, three nucleotides from X₁, X₂, X₁₉, and X₂₀ contain a 3′-PS group, respectively. In some embodiments, each X₁, X₂, X₁₉, and X₂₀ contains a 3′-PS group. In some embodiments, each X₁ and X₂ contains a 3′-PS group.

In some embodiments, at least four from X₁, X₂, X₃, X₄, X₁₇, X₁₈, X₁₉, and/or X₂₀ contain 3′-PS groups. In some embodiments, four from X₁, X₂, X₃, X₄, X₁₇, X₁₈, X₁₉, and/or X₂₀ contain a 3′-PS group, respectively. In some embodiments, six from X₁, X₂, X₃, X₄, X₁₇, X₁₈, X₁₉, and/or X₂₀ contain a 3′-PS group, respectively. In some embodiments, X₁, X₂, X₃, X₄, X₁₇, X₁₈, X₁₉, and X₂₀ contain a 3′-PS group, respectively.

In some embodiments, in X₃ to X₁₈, two to six nucleotides contain 3′-PS groups. In some embodiments, in X₃ to X₁₈, two nucleotides contain a 3′-PS group, respectively, respectively. In some embodiments, in X₃ to X₁₈, three nucleotides contain a 3′-PS group, respectively. In some embodiments, in X₃ to X₁₈, four nucleotides contain a 3′-PS group, respectively. In some embodiments, in X₃ to X₁₈, five nucleotides contain a 3′-PS group, respectively. In some embodiments, in X₃ to X₁₈, six nucleotides contain a 3′-PS group, respectively.

In certain aspects, the sense strand includes 2′-MOE modified nucleotides positioned at the 1^{st}, 2^{nd}, 20^{th}, and 21^{st} nucleotides from the 5′-end of the sense strand.

In some embodiments, the sense strand having 21 nucleotides in length includes:
(i) 2′-MOE modifications at the 1^{st}, 2^{nd}, 20^{th} and/or 21^{st} nucleotides from the 5′-end of the sense strand; and
(ii) 3′-PS modifications at the 1^{st}, 2^{nd}, 19^{th}, and/or 20^{th} nucleotides from 5′-end of the sense strand.

In some embodiments, the sense strand having 21 nucleotides in length includes:
(i) 2′-MOE modifications at the 1st, 2^{nd}, 20^{th} and/or 21^{st} nucleotides from the 5′-end of the sense strand; and 2′-F modifications at 8^{th}, 10^{th}, 11^{th}, and 12^{th} nucleotides from the 5′ end of the sense strand.

In some embodiments, the sense strand having 21 nucleotides in length includes:
(i) 2′-MOE modifications at the 1^{st}, 2^{nd}, 20^{th} and/or 21^{st} nucleotides from the 5′-end of the sense strand; 2′-F modifications at 7^{th}, 9^{th}, 10^{th}, and 11^{th} nucleotides from the 5′ end of the sense strand; and 2′-OMe modifications in the remaining nucleotides.

In some embodiments, the sense strand having 21 nucleotides in length includes:
(i) 2′-MOE modifications at the 1^{st}, 2^{nd}, 20^{th} and/or 21^{st} nucleotides from the 5′-end of the sense strand; and 2′-F modifications at 7^{th}, 9^{th}, 10^{th}, and 11^{th} nucleotides from the 5′ end of the sense strand, and
(ii) 3′-PS modifications at the 1^{st} and 2^{nd} nucleotides from 5′-end of the sense strand.

In some embodiments, the sense strand having 21 nucleotides in length includes:
(i) 2′-MOE modifications at the 1^{st}, 2^{nd}, 20^{th}, and 21^{st} nucleotides from the 5′-end of the sense strand; 2′-F modifications at 7^{th}, 9^{th}, 10^{th}, and/or 11^{th} nucleotides from the 5′ end of the sense strand; and 2′-OMe modifications in the remaining nucleotides, and
(ii) 3′-PS modifications at the 1^{st} and 2^{nd} nucleotides from 5′-end of the sense strand.

In some embodiments, the sense strand having 21 nucleotides in length includes:
(i) 2′-MOE modifications at the 1^{st}, 2^{nd}, 20^{th} and/or 21^{st} nucleotides from the 5′-end of the sense strand; and 2′-F modifications at 7^{th}, 9^{th}, 10^{th}, and 11^{th} nucleotides from the 5′ end of the sense strand, and
(ii) 3′-PS modifications at the 1^{st}, 2^{nd}, 19^{th}, and 20^{th} nucleotides from 5′ end of the sense strand.

In some embodiments, the sense strand having 21 nucleotides in length includes:
(i) 2′-MOE modifications at the 1^{st}, 2^{nd}, 20^{th}, and 21^{st} nucleotides from the 5′-end of the sense strand; 2′-F modifications at 7^{th}, 9^{th}, 10^{th}, and/or 11^{th} nucleotides from the 5′ end of the sense strand; and 2′-OMe modifications in the remaining nucleotides, and
(ii) 3′-PS modifications at the 1^{st}, 2^{nd}, 19^{th}, and 20^{th} nucleotides from 5′ end of the sense strand.

In certain aspects, a sense strand of the dsRNA as described herein may have a Formula (I′),

5′-Y_{1′}-Y_{2′}-Y₃-Y₄-Y₅-Y₆-Y₇-Y₈-Y₉-Y₁₀-Y₁₁-Y₁₂-Y₁₃-
Y₁₄-Y₁₅-Y₁₆-Y₁₇-Y₁₈-Y₁₉-Y₂₀-Y₂₁-3′ (I′)

wherein:
each Y₁, Y₂, Y₂₀, and Y₂₁ is independently a TNA; and
each Y₃ to Y₁₉ is independently selected from 2′-deoxy modified nucleotide, 2′-MOE modified nucleotide, 2′-F modified nucleotide, and 2′-OMe modified nucleotide.

In certain aspects, in Formula (I′), Y₃ to Y₁₉ do not include a 2′-MOE modified nucleotide. In some embodiments, each Y₃ to Y₁₉ is selected from deoxyribonucleotide, 2′-F modified nucleotides and 2′-OMe modified nucleotides. In some embodiments, at least one of Y₃ to Y₁₉ is not a deoxyribonucleotide.

In some embodiments, each Y_{f}, Y_{f+2}, and Y_{f+3} is 2′-F modified nucleotide and Y_{f+4} is 2′-deoxy modified nucleotide when f is an integer from 3 to 17. In some embodiments, f is 5. In some embodiments, f is 6. In some embodiments, f is 7. In some embodiments, f is 8. In some embodiments, f is 9. In some embodiments, Y₅, Y₇, and Y₈ are 2′-F modified nucleotides, and Y₉ is 2′-deoxy modified nucleotide (e.g., dT). In some embodiments, Y₆, Y₈, and Y₉ are 2′-F modified nucleotides and Y₁₀ is 2′-deoxy modified nucleotide (e.g., dT). In some embodiments, Y₇, Y₉, and Y₁₀ are 2′-F modified nucleotides, and Y₁₁ is 2′-deoxy modified nucleotide (e.g., dT). In some embodiments, Y₅, Y₁₀, and Y₁₁ are 2′-F modified nucleotides, and Y₁₂ is 2′-deoxy modified nucleotide (e.g., dT). In some embodiments, Y₉, Y₁₁, and Y₁₂ are 2′-F modified nucleotides, and Y₁₃ is 2′-deoxy modified nucleotide (e.g., dT).

In some embodiments, the sense strand includes 2′-OMe modified nucleotides in the remaining positions in the sense strand.

In some embodiments, at least two nucleotides from Y₁, Y₂, Y₁₉, and Y₂₀ contain a 3′-PS group, respectively. In some embodiments, two from Y₁, Y₂, Y₁₉, and Y₂₀ contains 3′-PS group. In some embodiments, three nucleotides from Y₁, Y₂, Y₁₉, and Y₂₀ contain a 3′-PS group, respectively. In some embodiments, each Y₁, Y₂, Y₁₉, and Y₂₀ contains a 3′-PS group. In some embodiments, each Y₁ and Y₂ contains a 3′-PS group.

In some embodiments, at least four from Y₁, Y₂, Y₃, Y₄, Y₁₇, Y₁₈, Y₁₉, and/or Y₂₀ contain 3′-PS groups. In some embodiments, four from Y₁, Y₂, Y₃, Y₄, Y₁₇, Y₁₈, Y₁₉, and/or Y₂₀ contain a 3′-PS group, respectively. In some embodiments, six from Y₁, Y₂, Y₃, Y₄, Y₁₇, Y₁₈, Y₁₉, and/or Y₂₀ contain a 3′-PS group, respectively. In some embodiments, Y₁, Y₂, Y₃, Y₄, Y₁₇, Y₁₈, Y₁₉, and Y₂₀ contain a 3′-PS group, respectively.

In some embodiments, in Y₃ to Y₁₈, two to six nucleotides contain 3′-PS groups. In some embodiments, in Y₃ to Y₁₈, two nucleotides contain a 3′-PS group, respectively. In some embodiments, in Y₃ to Y₁₈, three nucleotides contain a 3′-PS group, respectively. In some embodiments, in Y₃ to Y₁₈, four nucleotides contain a 3′-PS group, respectively. In some embodiments, in Y₃ to Y₁₈, five nucleotides contain a 3′-PS group, respectively. In some embodiments, in Y₃ to Y₁₈, six nucleotides contain a 3′-PS group, respectively.

In certain aspects, the sense strand includes TNAs positioned at the 1st, 2nd, 20th, and 21^{st} nucleotides from the 5′ end of the sense strand.

In some embodiments, the sense strand having 21 nucleotides in length includes:
(i) TNAs at the 1st, 2nd, 20th and/or 21st nucleotides from the 5′ end of the sense strand; and
(ii) 3′-PS modifications at the 1st, 2nd, 19th, and/or 20th nucleotides from 5′ end of the sense strand.

In some embodiments, the sense strand having 21 nucleotides in length includes:
(i) TNAs at the 1st, 2nd, 20th and/or 21st nucleotides from the 5′ end of the sense strand; 2′-F modifications at 7th, 9th, 10th, and 11th nucleotides from the 5′ end of the sense strand; and 2′-OMe modifications in the remaining nucleotides.

In some embodiments, the sense strand having 21 nucleotides in length includes:
(i) TNAs at the 1st, 2nd, 20th and/or 21st nucleotides from the 5′ end of the sense strand; 2′-F modifications at 7th, 9th, 10th and 11th nucleotides from the 5′ end of the sense strand; and 2′-OMe modifications in the remaining nucleotides; and
(ii) 3′-PS modifications at the 1st and 2nd nucleotides from 5′ end of the sense strand.

In some embodiments, the sense strand having 21 nucleotides in length includes:
(i) TNAs at the 1st, 2nd, 20th and/or 21st nucleotides from the 5′ end of the sense strand; 2′-F modifications at 7th, 9th, 10th and 11th nucleotides from the 5′ end of the sense strand; and 2′-OMe modifications in the remaining nucleotides; and
(ii) 3′-PS modifications at the 1st, 2nd, 19th, and 20th nucleotides from 5′ end of the sense strand.

In some embodiments, the sense strand having 21 nucleotides in length includes:
(i) TNAs at the 1st, 2nd, 20th and 21st nucleotides from the 5′ end of the sense strand; 2′-F modifications at 7th, 9th, 10th and 11th nucleotides from the 5′ end of the sense strand; and 2′-OMe modifications in the remaining nucleotides; and
(ii) 3′-PS modifications at the 1st and 2nd nucleotides from 5′ end of the sense strand.

In some embodiments, the sense strand having 21 nucleotides in length includes:
(i) TNAs at the 1st, 2nd, 20th and 21st nucleotides from the 5′ end of the sense strand; 2′-F modifications at 7th, 9th, 10th and 11th nucleotides from the 5′ end of the sense strand; and 2′-OMe modifications in the remaining nucleotides; and
(ii) 3′-PS modifications at the 1st, 2nd, 19th, and 20th nucleotides from 5′ end of the sense strand.

Example modification patterns of sense strands are shown in Table B.

TABLE B

| | 2′-MOE | | | 2′-OMe | |
|---|---|---|---|---|---|
| 21-mer SS | modified | | 2′F modified | modified | |
| modification | nucleotide | TNA | nucleotide | nucleotide | |
| pattern No. | position | position | position | position | 3′-PS linkage |
| SS1 | | | 7, 9, 10, 11 | 1, 2, 3, 4, 5, | 1, 2 |
| | | | | 6, 8, 12, 13, | |
| | | | | 14, 15, 16, | |
| | | | | 17, 18, 19, | |
| | | | | 20, 21 | |
| SS2 | 1, 2, 20, 21 | | 7, 9, 10, 11 | 3, 4, 5, 6, 8, | 1, 2 |
| | | | | 12, 13, 14, | |
| | | | | 15, 16, 17, | |
| | | | | 18, 19 | |
| SS3 | 1, 2, 20, 21 | | 7, 9, 10, 11 | 3, 4, 5, 6, 8, | 1, 2, 19, 20 |
| | | | | 12, 13, 14, | |
| | | | | 15, 16, 17, | |
| | | | | 18, 19 | |
| SS4 | | 1, 2, 20, 21 | 7, 9, 10, 11 | 3, 4, 5, 6, 8, | 1, 2 |
| | | | | 12, 13, 14, | |
| | | | | 15, 16, 17, | |
| | | | | 18, 19 | |
| SS5 | | 1, 2, 20, 21 | 7, 9, 10, 11 | 3, 4, 5, 6, 8, | 1, 2, 19, 20 |
| | | | | 12, 13, 14, | |
| | | | | 15, 16, 17, | |
| | | | | 18, 19 | |

In some embodiments, the sense strand having 21 nucleotides in length has the modification pattern of SS1. In some embodiments, the sense strand having 21 nucleotides in length does not have the modification pattern of SS1. In some embodiments, the sense strand having 21 nucleotides in length has the modification pattern of SS2. In some embodiments, the sense strand having 21 nucleotides in length has the modification pattern of SS3. In some embodiments, the sense strand having 21 nucleotides in length has the modification pattern of SS4. In some embodiments, the sense strand having 21 nucleotides in length has the modification pattern of SS5.

### Antisense Strand (AS)

In certain aspects, an antisense strand of the dsRNA as described herein are substantially (e.g., greater than about 80%, 85%, 90%, or 95% of the total nucleotides) made of modified nucleotides. In another certain aspect, the antisense strand is entirely made of modified nucleotides.

In certain aspects, the first nucleotide from the 5′ end of the antisense strand may contain an additional phosphate group or a variant thereof (e.g., phosphorothioate, phosphorodithioate, methylphosphonate, methylene phosphonate, or vinylphosphonate (VP)) attached or linked to the 5′ terminal group of the first nucleotide) attached or linked to the 5′ terminal group of the first nucleotide.

In certain aspects, the antisense strand includes 5′-vinylphosphonate (5′-VP) group at the first nucleotide from the 5′ end in the antisense strand. The “5′-VP” is a chemical moiety having the structure of

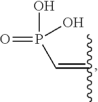 [SMILES: *=CP(=O)(O)O]

or a pharmaceutically acceptable salt thereof, where the wavy line represent the point of attachment to the 5′ carbon of the pentofuranosyl sugar of a nucleotide.

In some embodiments, the first nucleotide from the 5′ end in the antisense strand includes (E)-vinylphosphonate (VP) having a structure of

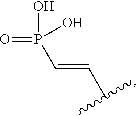 [SMILES: */C=C/P(=O)(O)O]

or a pharmaceutically acceptable salt thereof, wherein the wavy line presents the point of attachment to the 4′ carbon of the pentofuranosyl sugar of a nucleotide. In some embodiments, the first nucleotide from the 5′ end in the antisense strand includes (Z)-vinylphosphonate having a structure of

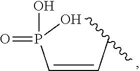 [SMILES: */C=C\P(=O)(O)O]

or a pharmaceutically acceptable salt thereof, wherein the wavy line presents the point of attachment to the 4′ carbon of the pentofuranosyl sugar of a nucleotide.

In some embodiments, the first nucleotide from the 5′ end of the antisense strand has a structure of

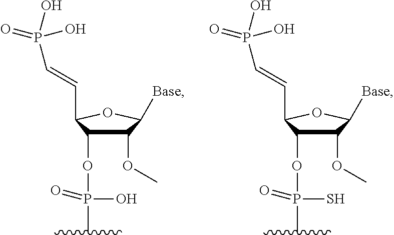

or a pharmaceutically acceptable salt thereof. In some embodiments, the first nucleotide from the 5′ end of the antisense strand has a structure of

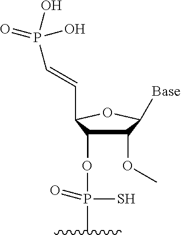

or a pharmaceutically acceptable salt thereof. In some embodiments, the first nucleotide from the 5′ end of the antisense strand has a structure of

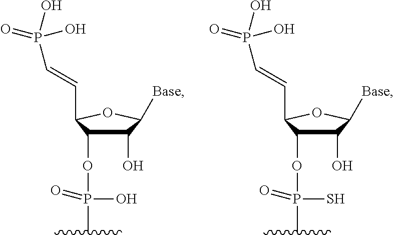

or a pharmaceutically acceptable salt thereof. In some embodiments, the first nucleotide from the 5′ end of the antisense strand has a structure of

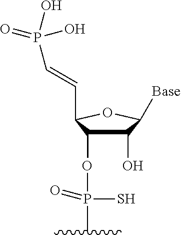

or a pharmaceutically acceptable salt thereof.

In some embodiments, the first nucleotide from the 5′ end of the antisense strand has a structure of

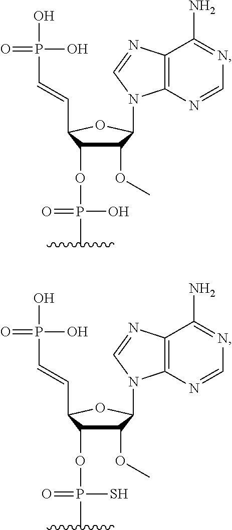

or a pharmaceutically acceptable salt thereof, wherein [symbol] is an attachment point to the adjacent nucleotides. In some embodiments, the first nucleotide from the 5′ end of the antisense strand has a structure of

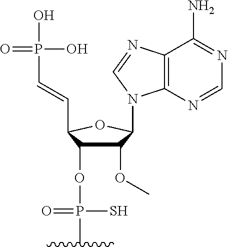

or a pharmaceutically acceptable salt thereof. In some embodiments, the first nucleotide from the 5′ end of the antisense strand has a structure of

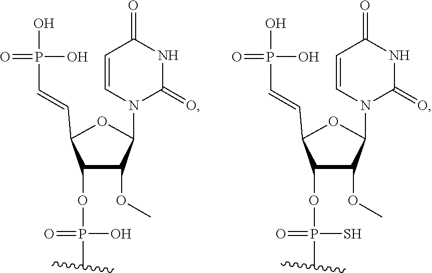

or a pharmaceutically acceptable salt thereof, wherein [symbol] is an attachment point to the adjacent nucleotides. In some embodiments, the first nucleotide from the 5′ end of the antisense strand has a structure of

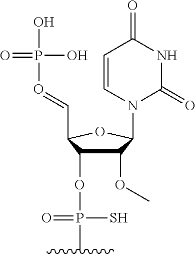

or a pharmaceutically acceptable salt thereof.

In certain aspects, the antisense strand of the dsRNA as described herein includes two or more 2′-F modifications. In some embodiments, the antisense strand of the dsRNA includes two, three, four, five, six, seven, or eight 2′-F modified nucleotides. In some embodiments, the antisense strand includes two 2′-F modified nucleotides. In some embodiments, the antisense strand includes three 2′-F modified nucleotides. In some embodiments, the antisense strand includes four 2′-F modified nucleotides. In some embodiments, the antisense strand includes five 2′-F modified nucleotides. In some embodiments, the antisense strand includes six 2′-F modified nucleotides. In some embodiments, the antisense strand includes seven 2′-F modified nucleotides. In some embodiments, the antisense strand includes eight 2′-F modified nucleotides. In some embodiments, two contiguous 2′-F modified nucleotides locate in the antisense strand. In some embodiments, three contiguous 2′-F modified nucleotides locate in the antisense strand. In some embodiments, four contiguous 2′-F modified nucleotides locate in the antisense strand.

In certain aspects, the antisense strand is 23 nucleotides in length. In some embodiments, the antisense includes comprises two, three, or four 2′-F modifications positioned at the 2nd, 6th, 14th, and/or 16th nucleotide from 5′ end of the antisense strand. In some embodiments, the antisense includes two 2′-F modifications positioned at the 2nd, 6th, 14th, and/or 16th nucleotide from 5′ end of the antisense strand. In some embodiments, the antisense includes three 2′-F modifications positioned at the 2nd, 6th, 14th, and/or 16th nucleotide from 5′ end of the antisense strand. In some embodiments, the antisense includes 2′-F modifications positioned at the 2nd, 6th, 14th, and 16th nucleotide from 5′ end of the antisense strand.

In certain aspects, an antisense strand of the dsRNA as described herein does not include a 2′-MOE modification. Alternatively, in certain aspects, the antisense strand includes one to four 2′-MOE modified nucleotides. In some embodiments, the antisense strand includes one 2′-MOE modified nucleotide. In some embodiments, the antisense strand includes two 2′-MOE modified nucleotides. In some embodiments, the antisense strand includes three 2′-MOE modified nucleotides. In some embodiments, the antisense strand includes four 2′-MOE modified nucleotides.

In certain aspects, the antisense strand is 23 nucleotides in length. In some embodiments, the antisense strand includes one to four 2′-MOE modification at the 1st, 9th, 10th, and 23rd nucleotides from the 5′-end of the antisense strand. In some embodiments, the antisense strand includes one 2′-MOE modification at the 1st, 9th, 10th, or 23rd nucleotides from the 5′-end of the antisense strand. In some embodiments, the antisense strand includes two 2′-MOE modifications at the 1st, 9th, 10th, and/or 23rd nucleotides from the 5′-end of the antisense strand. In some embodiments, the antisense strand includes three 2′-MOE modifications at the 1st, 9th, 10th, and/or 23rd nucleotides from the 5′-end of the antisense strand. In some embodiments, the antisense strand includes four 2′-MOE modification at the 1st, 9th, 10th, and 23rd nucleotides from the 5′-end of the antisense strand.

In certain aspects, the antisense strand includes at least one GNA nucleotide. In some embodiments, the antisense strand includes only one GNA.

In certain aspects, the antisense strand is 23 nucleotides in length. In some embodiments, the antisense strand includes only one GNA at the 4th nucleotide from 5′-end of the antisense strand. In some embodiments, the antisense strand includes only one GNA at the 5th nucleotide from 5′-end of the antisense strand. In some embodiments, the antisense strand includes only one GNA at the 6th nucleotide from 5′-end of the antisense strand.

In certain aspects, the antisense strand includes two, three, or four phosphorothioate (PS) linkages between nucleosides. In certain aspects, the antisense strand is 23 nucleotides in length. In some embodiments, the antisense strand includes two 3′-PS modifications positioned at the 1st, 2nd, 21st, and/or 22nd nucleotides from 5′-end of the antisense strand. In some embodiments, the antisense strand includes three 3′-PS modifications positioned at the 1st, 2nd, 21st, and/or 22nd nucleotides from 5′-end of the antisense strand. In some embodiments, the antisense strand includes 3′-PS modifications positioned at the 1st, 2nd, 21st, and 22nd nucleotides from 5′-end of the antisense strand.

In certain aspects, the antisense strand includes two to eight phosphorothioate (PS) linkages between nucleosides.

In certain aspects, the antisense strand is 23 nucleotides in length. In some embodiments, the antisense strand includes two 3′-PS modified nucleotides positioned at the 1st, 2nd, 3rd, 4th, 19th, 20th, 21st, and/or 22nd nucleotides from 5′-end of the antisense strand. In some embodiments, the antisense strand includes four 3′-PS modified nucleotides positioned at the 1st, 2nd, 3rd, 4th, 19th, 20th, 21st, and/or 22nd nucleotides from 5′-end of the antisense strand. In some embodiments, the antisense strand includes six 3′-PS modified nucleotides positioned at the 1st, 2nd, 3rd, 4th, 19th, 20th, 21st, and/or 22nd nucleotides from 5′-end of the antisense strand. In some embodiments, the antisense strand includes 3′-PS modified nucleotides positioned at the 1st, 2nd, 3rd, 4th, 19th, 20th, 21st, and 22nd nucleotides from 5′-end of the antisense strand.

In certain aspects, the antisense strand is 23 nucleotides in length. In some embodiments, at least one of the PS groups at the 1st, 2nd, 3rd, 4th, 19th, 20th, 21st, and/or 22nd nucleotides from 5′-end of the antisense strand is a stereopure Rp isomer. In some embodiments, at least one of the PS groups at the 1st, 2nd, 21st, and/or 22nd nucleotides from 5′-end of the antisense strand is a stereopure Rp isomer. In some embodiments, at least one of the PS groups at the 1st and/or 22nd nucleotides from 5′-end of the antisense strand is a stereopure Rp isomer. In some embodiments, the PS group at the 1st nucleotide from 5′-end of the antisense strand is a stereopure Rp isomer. In some embodiments, the PS group at the 22nd nucleotide from 5′-end of the antisense strand is a stereopure Rp isomer. In some embodiments, the PS groups at the 1st and 22nd nucleotides from 5′-end of the antisense strand are stereopure Rp isomers.

In certain aspects, the antisense strand is 23 nucleotides in length. In some embodiments, at least one of the PS groups at the 1st, 2nd, 3rd, 4th, 19th, 20th, 21st, and/or 22nd nucleotides from 5′-end of the antisense strand is a stereopure Sp isomer. In some embodiments, at least one of the PS groups at the 1st, 2nd, 21st, and/or 22nd nucleotides from 5′-end of the antisense strand is a stereopure Sp isomer. In some embodiments, at least one of the PS groups at the 1st and/or 22nd nucleotides from 5′-end of the antisense strand is a stereopure Sp isomer. In some embodiments, the PS group at the 1st nucleotide from 5′-end of the antisense strand is a stereopure Sp isomer. In some embodiments, the PS group at the 22nd nucleotide from 5′-end of the antisense strand is a stereopure Sp isomer. In some embodiments, the PS groups at the 1st and 22nd nucleotides from 5′-end of the antisense strand are stereopure Sp isomers.

In certain aspects, the antisense strand is 23 nucleotides in length. In some embodiments, at least one of the PS groups at the 1st, 2nd, 3rd, 4th, 19th, 20th, 21st, and/or 22nd nucleotides from 5′-end of the antisense strand is stereopure Sp isomer

In certain aspects, an antisense strand of dsRNA may have a Formula (II):

5′-X_{1′}-X_{2′}-X_{3′}-X_{4′}-X_{5′}-X_{6′}-X_{7′}-X_{8′}-X_{9′}-X_{10′}-X_{11′}-
X_{12′}-X_{13′}-X_{14′}-X_{15′}-X_{16′}-X_{17′}-X_{18′}-X_{19′}-X_{20′}-X_{21′}-
X_{22′}-X_{23′} -3′ (II)

wherein:
each X_{1′} to X_{23′} is independently selected from a 2′-deoxy modified nucleotide (deoxyribonucleotide), 2′-F modified nucleotide, 2′-OMe modified nucleotide, 2′-MOE modified nucleotide, and GNA; and
X_{1′} further includes a 5′-(E)-vinylphosphonate group.

In certain aspects, X_{1′} to X_{23′} do not include a 2′-MOE modified nucleotide. In some embodiments, each X_{1′} to X_{23′} is independently selected from 2′-F modified nucleotides and 2′-OMe modified nucleotides.

Alternatively, in certain aspect, X_{1′} to X_{23′} include one to four 2′-MOE modified nucleotides. In some embodiments, one of X_{1′}, X_{9′}, X_{10′}, and X_{23′} may be 2′-MOE modified nucleotide. In some embodiments, two of X_{1′}, X_{9′}, X_{10′}, and X_{23′} may be 2′-MOE modified nucleotides. In some embodiments, three of X_{1′}, X_{9′}, X_{10′}, and X_{23′} may be 2′-MOE modified nucleotides. In some embodiments, X_{1′}, X_{9′}, X_{10′}, and X_{23′} may be 2′-MOE modified nucleotide.

In some embodiments, X_{2′} is a 2′-F modified nucleotide. In some embodiments, X_{6′} is a 2′-F modified nucleotide. In some embodiments, X_{14′}is a 2′-F modified nucleotide. In some embodiments, X_{16′} is a 2′-F modified nucleotide. In some embodiments, two of X_{2′}, X_{6′}, X_{14′} and X_{16′} are 2′-F modified nucleotides. In some embodiments, three of X_{2′}, X_{6′}, X_{14′} and X_{16′} are 2′-F modified nucleotides. In some embodiments, each X_{2′}, X_{6′}, X_{14′} and X_{16′} is a 2′-F modified nucleotide.

In some embodiments, X_{1′} to X_{23′} may include at least one GNA. In some embodiments, X_{1′} to X_{23′} may include only one GNA. In some embodiments, X_{5′} is a GNA.

In some embodiments, the antisense strand includes 2′-OMe modified nucleotides in the remaining positions in the antisense strand.

In some embodiments, at least two from X_{1′}, X_{2′}, X_{21′}, and X_{22′} contain 3′-PS groups. In some embodiments, two from X_{1′}, X_{2′}, X_{21′}, and X_{22′} contain a 3′-PS group, respectively. In some embodiments, three from X_{1′}, X_{2′}, X_{21′}, and X_{22′} contain a 3′-PS group. In some embodiments, each X_{1′}, X_{2′}, X_{21′}, and X_{22′} contains a 3′-PS group.

In some embodiments, at least four from X_{1′}, X_{2′}, X_{3′}, X_{4′}, X_{19′}, X_{20′}, X_{21′}, and X_{22′} contain 3′-PS groups. In some embodiments, four from X_{1′}, X_{2′}, X_{3′}, X_{4′}, X_{19′}, X_{20′}, X_{21′}, and X_{22′} contain a 3′-PS group, respectively. In some embodiments, six from X_{1′}, X_{2′}, X_{3′}, X_{4′}, X_{19′}, X_{20′}, X_{21′}, and X_{22′} contain a 3′-PS group, respectively. In some embodiments, X_{1′}, X_{2′}, X_{3′}, X_{4′}, X_{19′}, X_{20′}, X_{21′}, and X_{22′} contain a 3′-PS group, respectively.

In some embodiments, in X_{3′} to X_{20′}, two to six nucleotides contain 3′-PS groups. In some embodiments, in X_{3′} to X_{20′}, two nucleotides contain a 3′-PS group, respectively. In some embodiments, in X_{3′} to X_{20′}, three nucleotides contain a 3′-PS group, respectively. In some embodiments, in X_{3′} to X_{20′}, four nucleotides contain a 3′-PS group, respectively. In some embodiments, in X_{3′} to X_{20′}, five nucleotides contain a 3′-PS group, respectively. In some embodiments, in X_{3′} to X_{20′}, six nucleotides contain a 3′-PS group, respectively.

In certain aspects, the antisense strand includes 5′-(E)-VP modified nucleotide at the first nucleotide from 5′ end of the antisense strand. In some embodiments, the antisense strand includes a 5′-(E)-VP-2′-OMe modified nucleotide at the first nucleotide from 5′ end of the antisense strand.

In some embodiments, the antisense strand having 23 nucleotides in length includes:
(i) a 5′-(E)-VP-2′-OMe modification at the first nucleotide from 5′ end of the antisense strand; and
(ii) 3′-PS modifications at the 1^{st}, 2^{nd}, 21^{st}, and/or 22^{nd} nucleotides from 5′-end of the antisense strand.

In some embodiments, the antisense strand having 23 nucleotides in length includes:
(i) a 5′-(E)-VP-2′-OMe modification at the first nucleotide from 5′ end of the antisense strand; and
(ii) 2′-F modifications at 2^{nd}, 6^{th}, 14^{th}, and/or 16^{th} nucleotides from the 5′ end of the antisense strand.

In some embodiments, the antisense strand having 23 nucleotides in length includes:
(i) a 5′-(E)-VP-2′-OMe modification at the first nucleotide from 5′ end of the antisense strand, and
(ii) 2′-F modifications at 2^{nd}, 6^{th}, 14^{th}, and/or 16^{th} nucleotides from the 5′ end of the antisense strand; and 2′-OMe modifications in the remaining nucleotides.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; and
(iii) 3′-PS modifications at the 1^{st}, 2^{nd}, 21^{st}, and/or 22^{nd} nucleotides from 5′-end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modification at 2^{nd}, 6^{th}, 14^{th}, and/or 16^{th} nucleotides from the 5′ end of the antisense strand; and 2′-OMe modification in the remaining nucleotides, and
(iii) 3′-PS modification at the 1^{st}, 2^{nd}, 21^{st}, and/or 22^{nd} nucleotides from 5′-end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modification at 2^{nd}, 6^{th}, 14^{th}, and 16^{th} nucleotides from the 5′ end of the antisense strand; and 2′-OMe modification in the remaining nucleotides, and
(iii) 3′-PS modification at the 1^{st}, 2^{nd}, 21^{st}, and 22^{nd} nucleotides from 5′-end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; GNA at 5^{th} nucleotide from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 14^{th}, and/or 16^{th} nucleotides from the 5′ end of the antisense strand; GNA at 6^{th} nucleotide from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; GNA at 7^{th} nucleotide from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; TNA at 3^{rd} nucleotide from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; TNA at 5^{th} nucleotide from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 14^{th}, and/or 16^{th} nucleotides from the 5′ end of the antisense strand; TNA at 6^{th} nucleotide from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; TNA at 7^{th} nucleotide from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; a 2′-deoxy modification at 5^{th} nucleotide from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 14^{th}, and/or 16^{th} nucleotides from the 5′ end of the antisense strand; a 2′-deoxy modification at 6^{th} nucleotide from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; a 2′-deoxy modification at 7^{th} nucleotide from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; GNA at 3^{rd} and 5^{th} nucleotides from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 14^{th}, and/or 16^{th} nucleotides from the 5′ end of the antisense strand; GNA at 3^{rd} and 6^{th} nucleotides from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16th nucleotides from the 5′ end of the antisense strand; GNA at 3rd and 7^{th} nucleotides from the 5′ end of the antisense strand; and 2′-OMe modifications in the remaining nucleotides; and
(iii) 3′-PS modifications at the It, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; TNA at 3^{rd} and 5^{th} nucleotides from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 14^{th}, and/or 16^{th} nucleotides from the 5′ end of the antisense strand; TNA at 3^{rd} and 6^{th} nucleotides from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; TNA at 3^{rd} and 7^{th} nucleotides from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; a 2′-deoxy modification at 3^{rd} and 5^{th} nucleotides from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 14^{th}, and/or 16^{th} nucleotides from the 5′ end of the antisense strand; a 2′-deoxy modification at 3^{rd} and 6^{th} nucleotide from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

In some embodiments, the antisense strand having 23 nucleotides in length includes:
(i) a 5′-(E)-VP-2′-OMe modification at the first nucleotide from 5′ end of the antisense strand;
(ii) 2′-F modifications at 2^{nd}, 6^{th}, 14^{th}, and/or 16^{th} nucleotides from the 5′ end of the antisense strand; a 2′-deoxy modification at 3^{rd} and 7^{th} nucleotides from the 5′ end of the antisense strand; and 2′-OMe modifications in the remaining nucleotides; and
(iii) 3′-PS modifications at the 1^{st}, 2^{nd}, 21^{st}, and/or 22^{nd} nucleotides from 5′ end of the antisense strand.

TABLE C

| | 5′-VP | | | | 2′-deoxy | 2′-OMe | |
|---|---|---|---|---|---|---|---|
| 23-mer AS | modified | 2′-F | | | modified | modified | |
| modification | nucleotide | modified | GNA | TNA | nucleotide | nucleotide | 3′-PS |
| pattern | position | nucleotide | position | position | position | position | linkage |
| AS1 | — | 2, 6, 14, 16 | | | | 1, 3, 4, 5, 7, 8, | 1, 2, 21, 22 |
| | | | | | | 9, 10, 11, 12, | |
| | | | | | | 13, 15, 17, 18, | |
| | | | | | | 19, 20, 21, 22, | |
| | | | | | | 23 | |
| AS2 | 1 | 2, 6, 14, 16 | | | | 1, 3, 4, 5, 7, 8, | 1, 2, 21, 22 |
| | | | | | | 9, 10, 11, 12, | |
| | | | | | | 13, 15, 17, 18, | |
| | | | | | | 19, 20, 21, 22, | |
| | | | | | | 23 | |
| AS3 | 1 | 2, 6, 14, 16 | 5 | | | 1, 3, 4, 7, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS4 | 1 | 2, 14, 16 | 6 | | | 1, 3, 4, 5, 7, 8, | 1, 2, 21, 22 |
| | | | | | | 9, 10, 11, 12, | |
| | | | | | | 13, 15, 17, 18, | |
| | | | | | | 19, 20, 21, 22, | |
| | | | | | | 23 | |
| AS5 | 1 | 2, 6, 14, 16 | 7 | | | 1, 3, 4, 5, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS6 | 1 | 2, 6, 14, 16 | | 3 | | 1, 4, 5, 7, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS7 | 1 | 2, 6, 14, 16 | | 5 | | 1, 3, 4, 7, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS8 | 1 | 2, 14, 16 | | 6 | | 1, 3, 4, 5, 7, 8, | 1, 2, 21, 22 |
| | | | | | | 9, 10, 11, 12, | |
| | | | | | | 13, 15, 17, 18, | |
| | | | | | | 19, 20, 21, 22, | |
| | | | | | | 23 | |
| AS9 | 1 | 2, 6, 14, 16 | | 7 | | 1, 3, 4, 5, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS10 | 1 | 2, 6, 14, 16 | | | 5 | 1, 3, 4, 7, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS11 | 1 | 2, 14, 16 | | | 6 | 1, 3, 4, 5, 7, 8, | 1, 2, 21, 22 |
| | | | | | | 9, 10, 11, 12, | |
| | | | | | | 13, 15, 17, 18, | |
| | | | | | | 19, 20, 21, 22, | |
| | | | | | | 23 | |
| AS12 | 1 | 2, 6, 14, 16 | | | 7 | 1, 3, 4, 5, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS13 | 1 | 2, 6, 14, 16 | 3,5 | | | 1, 4, 7, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS14 | 1 | 2, 14, 16 | 3, 6 | | | 1, 4, 5, 7, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS15 | 1 | 2, 6, 14, 16 | 3, 7 | | | 1, 4, 5, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS16 | 1 | 2, 6, 14, 16 | | 3,5 | | 1, 4, 7, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS17 | 1 | 2, 14, 16 | | 3, 6 | | 1, 4, 5, 7, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS18 | 1 | 2, 6, 14, 16 | | 3, 7 | | 1, 4, 5, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS19 | 1 | 2, 6, 14, 16 | | | 3,5 | 1, 4, 7, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS20 | 1 | 2, 14, 16 | | | 3, 6 | 1, 4, 5, 7, 8, 9, | 1, 2, 21, 22 |
| | | | | | | 10, 11, 12, 13, | |
| | | | | | | 15, 17, 18, 19, | |
| | | | | | | 20, 21, 22, 23 | |
| AS21 | 1 | 2, 6, 14, 16 | | | 3, 7 | 1, 4, 5, 8, 9, | 1, 2, 21, 22 |
| | | | | | | 10, 11, 12, 13, | |
| | | | | | | 15, 17, 18, 19, | |
| | | | | | | 20, 21, 22, 23 | |

In an aspect, the dsRNAi agent having the nucleotides modification patterns as described herein can improve half-life relative to a reference dsRNAi agent that does not contain such nucleotides modification patterns. In some embodiments, the dsRNAi agent having the nucleotides modification patterns as described herein improved half-life by about 1.1 fold, 1.2 fold, 1.3 fold, 1.4 fold, 1.5 fold, 1.6 fold, 1.7 fold, 1.8 fold, 1.9 fold, 2.0 fold, 3.0 fold, 4.0 fold, 5.0 fold, 6.0 fold, 7.0 fold, 8.0 fold, 9.0 fold, 10 fold, or more relative to a reference dsRNAi agent that does not contain such nucleotides modification patterns. In some embodiments, the dsRNAi agent having the nucleotides modification patterns as described herein improved half-life by about 2 fold, 2.5 fold, 3 fold, 3.5 fold, 4 fold, 4.5 fold, 5 fold, 7 fold, or 10 fold, relative to a reference dsRNAi agent that does not contain such nucleotides modification patterns.

dsRNA Modification Pattern

In an aspect, the dsRNA as described herein includes a sense strand of Formula (I) as described herein and an antisense strand of Formula (II) as described herein. The sense strand and the antisense strand form a duplex.

In certain aspects, the dsRNA includes:
(i) a sense strand including 2′-MOE modifications at X₁, X₂, X₂₀ and X₂₁; and
(ii) an antisense strand including a 5′-(E)-VP-2′-OMe modification at X_{1′}.

In some embodiments, the dsRNA includes:
(i) a sense strand including:
   (a) 2′-MOE modifications at X₁, X₂, X₂₀ and X₂₁; and
   (b) 3′-PS modifications at X₁ and X₂, and
(ii) an antisense strand including:
   (a) a 5′-(E)-VP-2′-OMe modification at X_{1′}; and
   (b) 3′-PS modifications at X_{1′}, X_{2′}, X_{21′}, and X_{22′}.

In some embodiments, the dsRNA includes:
(i) a sense strand including:
   (a) 2′-MOE modifications at X₁, X₂, X₂₀ and X₂₁; and 2′-F modifications at X₇, X₉, X₁₀ and X₁₁, and
(ii) an antisense strand including:
   (a) a 5′-(E)-VP-2′-OMe modification at X_{1′}, and
   (b) 2′-F modifications at X_{2′}, X_{6′}, X_{14′} and/or X_{16′}.

In some embodiments, the dsRNA includes:
(i) a sense strand including:
   (a) 2′-MOE modifications at X₁, X₂, X₂₀ and X₂₁; and 2′-F modifications at X₇, X₉, X₁₀ and X₁₁, and
   (b) 3′-PS modifications at X₁ and X₂, and
(ii) an antisense strand including:
   (a) a 5′-(E)-VP-2′-OMe modification at X_{1′};
   (b) 2′-F modifications at X_{2′}, X_{6′}, X_{14′} and/or X_{16′}; and
   (c) 3′-PS modifications X_{1′}, X_{2′}, X_{21′}, and X_{22′}.

In some embodiments, the dsRNA includes:
(i) a sense strand including:
   (a) 2′-MOE modifications at X₁, X₂, X₂₀ and X₂₁; 2′-F modifications at X₇, X₉, X₁₀ and X₁₁; and 2′-OMe modifications in the remaining nucleotides in the sense strand, and
   (b) 3′-PS modifications at X₁ and X₂, and
(ii) an antisense strand including:
   (a) a 5′-(E)-VP-2′-OMe modification at X_{1′},
   (b) 2′-F modifications at X_{2′}, X_{6′}, X_{14′} and/or X_{11′}; and 2′-OMe modifications in the remaining nucleotides in the antisense strand, and
   (c) 3′-PS modifications at X_{1′}, X_{2′}, X_{21′}, and X_{22′}.

In some embodiments, the dsRNA includes:
(i) a sense strand including:
   (a) 2′-MOE modifications at X₁, X₂, X₂₀ and X₂₁; 2′-F modifications at X₇, X₉, X₁₀ and X₁₁; and 2′-OMe modifications in the remaining nucleotides in the sense strand, and
   (b) 3′-PS modifications at X₁ and X₂, and
(ii) an antisense strand including:
   (a) a 5′-(E)-VP-2′-OMe modification at X_{1′},
   (b) a GNA nucleotide at X_{5′},
   (c) 2′-F modifications at X_{2′}, X_{6′}, X_{14′} and/or X_{16′}; and 2′-OMe modifications in the remaining nucleotides in the antisense strand, and (d) 3′-PS modifications at X_{1′}, X_{2′}, X_{21′}, and X_{22′}.

In some embodiments, the dsRNA includes:
(i) a sense strand including:
   (a) 2′-MOE modifications at X₁, X₂, X₂₀ and X₂₁; 2′-F modifications at X₇, X₉, X₁₀ and X₁₁; and 2′-OMe modifications in the remaining nucleotides in the sense strand, and
   (b) 3′-PS modifications at X₁ and X₂, and
(ii) an antisense strand including:
   (a) a 5′-(E)-VP-2′-OMe modification at X_{1′};
   (b) 3′-PS modifications at X_{1′}, X_{2′}, X_{21′}, and X_{22′}; and
   (c) one selected from:
      (c1) 2′-F modifications at X_{2′}, X_{6′}, X_{14′} and X_{16′}; and 2′-OMe modifications in the remaining nucleotides in the antisense strand,
      (c2) 2′-F modifications at X_{2′}, X_{6′}, X_{14′} and X_{16′}; TNA at X_{3′}; and 2′-OMe modifications in the remaining nucleotides in the antisense strand,
      (c3) 2′-F modifications at X_{2′}, X_{6′}, X_{14′} and X_{16′}; TNA, GNA or 2′-deoxy modification at X₅; and 2′-OMe modifications in the remaining nucleotides in the antisense strand,
      (c4) 2′-F modifications at X_{2′}, X_{14′} and X_{16′}; TNA, GNA or 2′-deoxy modification at X_{6′}; and 2′-OMe modifications in the remaining nucleotides in the antisense strand, and
      (c5) 2′-F modifications at X_{2′}, X_{6′}, X_{14′} and X_{16′}; TNA, GNA or 2′-deoxy modification at X_{7′}; and 2′-OMe modifications in the remaining nucleotides in the antisense strand.

In some embodiments, the dsRNA includes:
(i) a sense strand of Formula (I′) including:
   (a) TNAs at Y₁, Y₂, Y₂₀ and Y₂₁; and
   (b) 3′-PS modifications at Y₁, Y₂, Y₁₉, and Y₂₀, and
(ii) an antisense strand of Formula (II) including:
   (a) a 5′-(E)-VP-2′-OMe modification at X_{1′}; and
   (b) 3′-PS modifications at X_{1′}, X_{2′}, X_{21′}, and X_{22′}.

In some embodiments, the dsRNA includes:
(i) a sense strand of Formula (I′) including:
   (a) TNAs at Y₁, Y₂, Y₂₀ and Y₂₁;
   (b) 3′-PS modifications at Y₁, Y₂, Y₁₉, and/or Y₂₀; and
   (c) 2′-F modifications at Y₇, Y₉, and Y₁₀; 2′-deoYy modification at Y₁₁; and 2′-OMe modifications in the remaining nucleotides in the sense strand, and
(ii) an antisense strand of Formula (II) including:
   (a) a 5′-(E)-VP-2′-OMe modification at X_{1′};
   (b) 3′-PS modifications at X_{1′}, X_{2′}, X_{21′}, and X_{22′}; and
   (c) one selected from:
      (c1) 2′-F modifications at X_{2′}, X_{6′}, X_{14′} and X_{16′}; and 2′-OMe modifications in the remaining nucleotides in the antisense strand,
      (c2) 2′-F modifications at X_{2′}, X_{6′}, X_{14′} and X_{16′}; TNA at X_{3′}; and 2′-OMe modifications in the remaining nucleotides in the antisense strand,
      (c3) 2′-F modifications at X_{2′}, X_{6′}, X_{14′} and X_{16′}; TNA, GNA or 2′-deoxy modification at X_{5′}; and 2′-OMe modifications in the remaining nucleotides in the antisense strand,
      (c4) 2′-F modifications at X_{2′}, X_{14′} and X_{16′}; TNA, GNA or 2′-deoxy modification at X_{6′}; and 2′-OMe modifications in the remaining nucleotides in the antisense strand, and
      (c5) 2′-F modifications at X_{2′}, X_{6′}, X_{14′} and X_{16′}; TNA, GNA or 2′-deoxy modification at X_{7′}; and 2′-OMe modifications in the remaining nucleotides in the antisense strand.

In certain aspects, the dsRNA includes:
(i) a sense strand having 21 nucleotides in length and including 2′-MOE modifications at the 1^{st}, 2^{nd}, 20^{th} and 21^{st} nucleotides from the 5′end of the sense strand; and
(ii) an antisense strand having 23 nucleotides in length and including a 5′-(E)-VP-2′-OMe modification at the 1^{st} nucleotide from 5′ end of the antisense strand.

In some embodiments, the dsRNA as described herein includes a sense strand having 21 nucleotides in length and an antisense strand having 23 nucleotides.

In some embodiments, the dsRNA as described herein includes a sense strand having 21 nucleotides in length and including 2′-MOE modifications at the 1^{st}, 2^{nd}, 20^{th} and 21^{st} nucleotides from the 5′end of the sense strand.

In some embodiments, the dsRNA as described herein includes antisense strand having 23 nucleotides in length and including a 5′-(E)-VP-2′-OMe modification at the 1st position from 5′ end of the antisense strand.

In some embodiments, the dsRNA has Modification Pattern A of:
(i) a sense strand having 21 nucleotides in length and including:
   (a) 2′-MOE modifications at the 1^{st}, 2^{nd}, 20^{th} and 21^{st} nucleotides from the 5′end of the sense strand, and
   (b) 3′-PS modifications at the 1^{st} and 2^{nd} nucleotides from 5′-end of the sense strand, and
(ii) an antisense strand having 23 nucleotides in length and including:
   (a) a 5′-(E)-VP-2′-OMe modification at the 1^{st} position from 5′ end of the antisense strand, and
   (b) 3′-PS modifications at the 1^{st}, 2^{nd}, 21^{st}, and 22^{nd} nucleotides from 5′-end of the antisense strand.

In some embodiments, the dsRNA has Modification Pattern B of:
(i) a sense strand having 21 nucleotides in length and including:
   (a) 2′-MOE modifications at the 1^{st}, 2^{nd}, 20^{th} and 21^{st} nucleotides from the 5′-end of the sense strand; and 2′-F modifications at the 7^{th}, 9^{th}, 10^{th}, and 11^{th} nucleotides from the 5′ end of the sense strand, and
(ii) an antisense strand having 23 nucleotides in length and including:
   (a) a 5′-(E)-VP-2′-OMe modification at the 1^{st} position from 5′ end of the antisense strand, and
   (b) 2′-F modifications at the 2^{nd}, 6^{th}, 14^{th}, and 16^{th} nucleotides from the 5′ end of the antisense strand.

In some embodiments, the dsRNA has Modification Pattern C of:
(i) a sense strand having 21 nucleotides in length and including:
   (a) 2′-MOE modifications at the 1^{st}, 2^{nd}, 20^{th} and 21^{st} nucleotides from the 5′-end of the sense strand; and 2′-F modifications at the 7^{th}, 90^{th}, and 11^{th} nucleotides from the 5′ end of the sense strand, and
   (b) 3′-PS modifications at the 1^{st} and 2^{nd} nucleotides from 5′-end of the sense strand, and
(ii) an antisense strand having 23 nucleotides in length and including:
   (a) a 5′-(E)-VP-2′-OMe modification at the 1^{st} position from 5′ end of the antisense strand,
   (b) 2′-F modifications at the 2^{nd}, 6^{th}, 14^{th}, and 16^{th} nucleotides from the 5′ end of the antisense strand, and (c) 3′-PS modifications at the 1^{st}, 2^{nd}, 21^{st}, and 22^{nd} nucleotides from 5′-end of the antisense strand.

In some embodiments, the dsRNA has Modification Pattern D of:
(i) a sense strand having 21 nucleotides in length and including:
   (a) 2′-MOE modifications at the 1^{st}, 2^{nd}, 20^{th} and 21^{st} nucleotides from the 5′-end of the sense strand; 2′-F modifications at 7^{th}, 9^{th}, 10^{th}, and 11^{th} nucleotides from the 5′ end of the sense strand; and 2′-OMe modifications in the remaining nucleotides in the sense strand, and
   (b) 3′-PS modifications at the 1^{st} and 2^{nd} nucleotides from 5′-end of the sense strand, and
(ii) an antisense strand having 23 nucleotides in length and including:
   (a) a 5′-(E)-VP-2′-OMe modification at the 1^{st} position from 5′ end of the antisense strand,
   (b) 2′-F modifications at 2^{nd}, 6^{th}, 14^{th}, and 16^{th} nucleotides from the 5′ end of the antisense strand; and 2′-OMe modifications in the remaining nucleotides in the sense strand, and
   (c) 3′-PS modifications at the 1^{st}, 2^{nd}, 21^{st}, and 22^{nd} nucleotides from 5′-end of the antisense strand.

In some embodiments, the dsRNA has Modification Pattern E of:
(i) a sense strand having 21 monomers (e.g., nucleotides) in length and including:
   (a) 2′-MOE modifications at the 1^{st}, 2^{nd}, 20^{th} and 21^{st} positions from the 5′-end of the sense strand; 2′-F modifications at 7^{th}, 9^{th}, 10^{th}, and 11^{th} positions from the 5′ end of the sense strand; and 2′-OMe modifications in the remaining positions in the sense strand, and
   (b) 3′-PS modifications at the 1^{st} and 2^{nd} positions from 5′-end of the sense strand; and
(ii) an antisense strand having 23 monomers (e.g., nucleotides) in length and including:
   (a) a 5′-(E)-VP-2′-OMe modification at the 1St position from 5′ end of the antisense strand,
   (b) a GNA at 5^{th} position from the 5′ end of the antisense strand; 2′-F modifications at 2^{nd}, 6^{th}, 14^{th}, and 16^{th} positions from the 5′ end of the antisense strand; and 2′-OMe modifications in the remaining positions in the antisense strand, and
   (c) 3′-PS modifications at the 1^{st}, 2^{nd}, 21^{st}, and 22^{nd} positions from 5′-end of the antisense strand.

In some embodiments, the dsRNA has Modification Pattern E-1 of:
(i) a sense strand having 21 monomers (e.g., nucleotides) in length and including:
   (a) 2′-MOE modifications at the 1^{st}, 2^{nd}, 20^{th} and 21^{st} positions from the 5′-end of the sense strand; 2′-F modifications at 7^{th}, 9^{th}, 10^{th}, and 11^{th} positions from the 5′ end of the sense strand; and 2′-OMe modifications in the remaining positions in the sense strand, and
   (b) 3′-PS modifications at the 1^{st} and 2^{nd} positions from 5′-end of the sense strand; and
(ii) an antisense strand having 23 monomers (e.g., nucleotides) in length and including:
   (a) a 5′-(E)-VP-2′-OMe modification at the 1^{st} position from 5′ end of the antisense strand,
   (b) a GNA, TNA, or 2′-deoxy modification at 5^{th} position from the 5′ end of the antisense strand; 2′-F modifications at 2^{nd}, 6^{th}, 14^{th}, and 16^{th} positions from the 5′ end of the antisense strand; and 2′-OMe modifications in the remaining positions in the antisense strand, and
   (c) 3′-PS modifications at the 1^{st}, 2^{nd}, 21^{st}, and 22nd positions from 5′-end of the antisense strand.

In some embodiments, the dsRNA has Modification Pattern E-2 of:
(i) a sense strand having 21 monomers (e.g., nucleotides) in length and including:
   (a) 2′-MOE modifications at the 1^{st}, 2^{nd}, 20^{th} and 21^{st} positions from the 5′-end of the sense strand; 2′-F modifications at 7^{th}, 9^{th}, 10^{th}, and 11^{th} positions from the 5′ end of the sense strand; and 2′-OMe modifications in the remaining positions in the sense strand, and
   (b) 3′-PS modifications at the 1^{st} and 2^{nd} positions from 5′-end of the sense strand; and
(ii) an antisense strand having 23 monomers (e.g., nucleotides) in length and including:
   (a) a 5′-(E)-VP-2′-OMe modification at the 1^{st} position from 5′ end of the antisense strand,
   (b) a TNA nucleotide at 3^{rd} position from the 5′ end of the antisense strand; 2′-F modifications at 2^{nd}, 6^{th}, 14^{th}, and 16^{th} positions from the 5′ end of the antisense strand; and 2′-OMe modifications in the remaining positions in the antisense strand, and (c) 3′-PS modifications at the 1^{st}, 2^{nd}, 21^{st}, and 22nd positions from 5′-end of the antisense strand.

In some embodiments, the dsRNA has Modification Pattern E-3 of:
(i) a sense strand having 21 monomers (e.g., nucleotides) in length and including:
   (a) 2′-MOE modifications at the 1^{st}, 2^{nd}, 20^{th} and 21^{st} positions from the 5′-end of the sense strand; 2′-F modifications at 7^{th}, 9^{th}, 10^{th}, and 11^{th} positions from the 5′ end of the sense strand; and 2′-OMe modifications in the remaining positions in the sense strand, and
   (b) 3′-PS modifications at the 1^{st} and 2^{nd} positions from 5′-end of the sense strand; and
(ii) an antisense strand having 23 monomers (e.g., nucleotides) in length and including:
   (a) a 5′-(E)-VP-2′-OMe modification at the 1^{st} position from 5′ end of the antisense strand,
   (b) a GNA, TNA, or 2′-deoxy modification at 6^{th} position from the 5′ end of the antisense strand; 2′-F modifications at 2^{nd}, 14^{th}, and 16^{th} positions from the 5′ end of the antisense strand; and 2′-OMe modifications in the remaining positions in the antisense strand, and
   (c) 3′-PS modifications at the 1^{st}, 2^{nd}, 21^{st}, and 22nd positions from 5′-end of the antisense strand.

In some embodiments, the dsRNA has Modification Pattern E-4 of:
(i) a sense strand having 21 monomers (e.g., nucleotides) in length and including:
   (a) 2′-MOE modifications at the 1^{st}, 2^{nd}, 20^{th} and 21^{st} positions from the 5′-end of the sense strand; 2′-F modifications at 7^{th}, 9^{th}, 10^{th}, and 11^{th} positions from the 5′ end of the sense strand; and 2′-OMe modifications in the remaining positions in the sense strand, and
   (b) 3′-PS modifications at the 1^{st} and 2^{nd} positions from 5′-end of the sense strand; and
(ii) an antisense strand having 23 monomers (e.g., nucleotides) in length and including:
   (a) a 5′-(E)-VP-2′-OMe modification at the 1^{st} position from 5′ end of the antisense strand,
   (b) a GNA, TNA, or 2′-deoxy modification at 7^{th} position from the 5′ end of the antisense strand; 2′-F modifications at 2^{nd}, 6^{th}, 14^{th}, and 16^{th} positions from the 5′ end of the antisense strand; and 2′-OMe modifications in the remaining positions in the antisense strand, and
   (c) 3′-PS modifications at the 1^{st}, 2^{nd}, 21^{st}, and 22nd positions from 5′-end of the antisense strand.

In certain aspects, the antisense strand of the dsRNA as described herein does not include a 5′-(E)-VP-2′-OMe modification at the 1^{st} position from 5′ end of the antisense strand.

In some embodiments, the dsRNA has Modification Pattern F of:
(i) a sense strand having 21 nucleotides in length and including:
   (a) 2′-MOE modifications at the 1^{st}, 2^{nd}, 20^{th} and 21^{st} nucleotides from the 5′-end of the sense strand; 2′-F modifications at 7^{th}, 9^{th}, 10^{th}, and 11^{th} nucleotides from the 5′ end of the sense strand; and 2′-OMe modifications in the remaining nucleotides in the sense strand, and
   (b) 3′-PS modifications at the 1^{st} and 2^{nd} nucleotides from 5′-end of the sense strand.
(ii) an antisense strand having 23 nucleotides in length and including:
   (a) 2′-F modifications at 2^{nd}, 6^{th}, 14^{th}, and 16^{th} nucleotides from the 5′ end of the antisense strand; and 2′-OMe modifications in the remaining nucleotides in the antisense strand, and
   (b) 3′-PS modifications at the 1^{st}, 2^{nd}, 21^{st}, and 22^{nd} nucleotides from 5′-end of the antisense strand.

In some embodiments, the dsRNA has Modification Pattern G-1 of:
(i) a sense strand having 21 nucleotides in length and including:
   (a) 2′-F modifications at 7^{th}, 9^{th}, 10^{th}, and 11^{th} nucleotides from the 5′ end of the sense strand; and 2′-OMe modifications in the remaining nucleotides in the sense strand, and
   (b) 3′-PS modifications at the 1^{st} and 2^{nd} nucleotides from 5′-end of the sense strand, and
(ii) an antisense strand having 23 nucleotides in length and including:
   (a) 2′-F modifications at 2^{nd}, 6^{th}, 14^{th}, and/or 16^{th} nucleotides from the 5′ end of the antisense strand; and 2′-OMe modifications in the remaining nucleotides in the antisense strand, and
   (b) 3′-PS modifications at the 1^{st}, 2^{nd}, 21^{st}, and 22^{nd} nucleotides from 5′-end of the antisense strand.

In some embodiments, the dsRNA has Modification Pattern G-2 of:
(i) a sense strand having 21 nucleotides in length and including:
   (a) 2′-F modifications at 7^{th}, 9^{th}, 10^{th}, and 11^{th} nucleotides from the 5′ end of the sense strand; and 2′-OMe modifications in the remaining nucleotides in the sense strand, and
   (b) 3′-PS modifications at the 1^{st} and 2^{nd} nucleotides from 5′-end of the sense strand, and
(ii) an antisense strand having 23 nucleotides in length and including:
   (a) 2′-F modifications at 2^{nd}, 6^{th}, 9^{th}, 14^{th}, and 16^{th} nucleotides from the 5′ end of the antisense strand; and 2′-OMe modifications in the remaining nucleotides in the antisense strand, and
   (b) 3′-PS modifications at the 1^{st}, 2^{nd}, 21^{st}, and 22^{nd} nucleotides from 5′-end of the antisense strand.

In some embodiments, the dsRNA has Modification Pattern H of:
(i) a sense strand having 21 nucleotides in length and including:
   (a) 2′-F modifications at 7^{th}, 9^{th}, 10^{th}, and 11^{th} nucleotides from the 5′ end of the sense strand; and 2′-OMe modifications in the remaining nucleotides in the sense strand, and
   (b) 3′-PS modifications at the 1^{st} and 2^{nd} nucleotides from 5′-end of the sense strand, and
(ii) an antisense strand having 23 nucleotides in length and including:
   (a) a 5′-(E)-VP-2′-OMe modification at the 1^{st} position from 5′ end of the antisense strand;
   (b) 2′-F modifications at 2^{nd}, 6^{th}, 14^{th}, and 16^{th} nucleotides from the 5′ end of the antisense strand; and 2′-OMe modifications in the remaining nucleotides in the antisense strand, and
   (c) 3′-PS modifications at the 1^{st}, 2^{nd}, 21^{st}, and 22^{nd} nucleotides from 5′-end of the antisense strand.

In some embodiments, the dsRNA has Modification Pattern I of:
(i) a sense strand having 21 nucleotides in length and including:
   (a) TNAs at the 1st, 2nd, 20th and 21st nucleotides from the 5′ end, and
   (b) 3′-PS modifications at the 1st and 2nd nucleotides from the 5′ end, and
(ii) an antisense strand having 23 nucleotides in length and including:
   (a) a 5′-(E)-VP-2′-OMe modification at the 1st position from the 5′ end, and
   (b) 3′-PS modifications at the 1st, 2nd, 21st, and 22nd nucleotides from the 5′ end.

In some embodiments, the dsRNA has Modification Pattern J of:
(i) a sense strand having 21 nucleotides in length and including:
   (a) TNAs at the 1st, 2nd, 20th and 21^{st} nucleotides from the 5′ end, and
   (b) 3′-PS modifications at the 1st, 2nd, 19th, and 20th nucleotides from the 5′ end, and
(ii) an antisense strand having 23 nucleotides in length and including:
   (a) a 5′-(E)-VP-2′-OMe modification at the 1^{st} position from the 5′ end, and
   (b) 3′-PS modifications at the 1st, 2nd, 21st, and 22nd nucleotides from the 5′ end.

In some embodiments, the dsRNA has Modification Pattern K of:
(i) a sense strand having 21 nucleotides in length and including:
   (a) TNAs at the 1st, 2nd, 20th and 21st nucleotides from the 5′ end,
   (b) 3′-PS modifications at the 1st, 2nd, 19th, and/or 20th nucleotides from the 5′ end, and
   (c) 2′-F modifications at the 7th, 9th, 10th, and 11th nucleotides from the 5′ end; and 2′-OMe modifications in the remaining nucleotides in the sense strand,
and
(ii) an antisense strand having 23 nucleotides in length and including:
   (a) a 5′-(E)-VP-2′-OMe modification at the 1^{st} position from the 5′ end,
   (b) 3′-PS modifications at the 1st, 2nd, 21st, and 22nd nucleotides from the 5′ end, and
   (c) one selected from:
      (c1) 2′-F modifications at the 2nd, 6th, 14th, and 16th nucleotides from the 5′ end; and 2′-OMe modifications in the remaining nucleotides in the antisense strand,
      (c2) 2′-F modifications at the 2nd, 6th, 14th, and 16th nucleotides from the 5′ end; TNA at the 3rd nucleotide from the 5′ end; and 2′-OMe modifications in the remaining nucleotides in the antisense strand,
      (c3) 2′-F modifications at the 2nd, 6th, 14th, and 16th nucleotides from the 5′ end; TNA, GNA or 2′-deoxy modification at the 5th nucleotide from the 5′ end; and 2′-OMe modifications in the remaining nucleotides in the antisense strand,
      (c4) 2′-F modifications at the 2nd, 14th, and 16th nucleotides from the 5′ end; TNA, GNA or 2′-deoxy modification at the 6th nucleotide from the 5′ end; and 2′-OMe modifications in the remaining nucleotides in the antisense strand, and
      (c5) 2′-F modifications at the 2nd, 6th, 14th, and 16th nucleotides from the 5′ end; TNA, GNA or 2′-deoxy modification at the 7th nucleotide from the 5′ end; and 2′-OMe modifications in the remaining nucleotides in the antisense strand.

In certain aspects, modified sequences of sense strands and antisense strands targeting the above indicated HMGCR mRNA (SEQ ID NO: 811, or GenBank: NM_000859.3) are in Table 3.

TABLE 3

| siRNA | | | SEQ ID |
|---|---|---|---|
| No. | Sequence (5′-3′) | Strand | NO. |
| 647 | T005p001G005p001U004pU004pG004pU004pC007pA004pA007 | SS | 1294 |
| | pG007pA007pC004pU004pU004pU004pU004pU004pC004pG004 | | |
| | pA005pA005 | | |
| | X033U1027p001U007p001C004pG004pA004pA007pA004pA004 | AS | 1298 |
| | pA004pG004pU004pC004pU004pU007pG004pA007pC004pA004 | | |
| | pA004pC004pA004p001U004p001U004 | | |
| 648 | T005p001T005p001G004pC004pA004pG004pA007pU004pG007 | SS | 1295 |
| | pC007pU007pA004pG004pG004pU004pG004pU004pU004pC004 | | |
| | pA005pA005 | | |
| | X033U1027p001U007p001G004pA004pA004pC007pA004pC004 | AS | 1299 |
| | pC004pU004pA004pG004pC004pA007pU004pC007pU004pG004 | | |
| | pC004pA004pA004p001A004p001C004 | | |
| 649 | T005p001C005*p001A004pA004pG004pA004pC007pU004pU00 | SS | 1296 |
| | 7pU007pU007pU004pC004pG004pA004pA004pU004pG004pC00 | | |
| | 4pA005pA005 | | |
| | X033U1027p001U007p001G004pC004pA1016pU004pU004pC00 | AS | 1300 |
| | 4pG004pA004pA004pA004pA004pA007pG004pU007pC004pU00 | | |
| | 4pU004pG004pA004p001C004p001A004 | | |
| 650 | T005p001T005p001G004pC004pA004pG004pA007pU004pG007 | SS | 1297 |
| | pC007pU007pA004pG004pG004pU004pG004pU004pU004pC004 | | |
| | pA005pT005 | | |
| | A004p001U007p001G004pA004pA004pC007pA004pC004pC004 | AS | 1301 |
| | pU004pA004pG004pC004pA007pU004pC007pU004pG004pC004 | | |
| | pA004pA004p001A004p001C004 | | |
| 709 | G005p001A005p001U004pU004pC004pU004pG007pU004pA007 | SS | 1448 |
| | pG007pC007pU004pA004pC004pA004pA004pU004pG004pU004 | | |
| | pT005pA005 | | |
| | X033U1027p001A007p001A004pC004pA004pU007pU004pG004 | AS | 1463 |
| | pU004pA004pG004pC004pU004pA007pC004pA007pG004pA004 | | |
| | pA004pU004pC004p001C004p001U004 | | |
| 710 | A005p001T005p001U004pC004pU004pG004pU007pA004pG007 | SS | 1449 |
| | pC007pU007pA004pC004pA004pA004pU004pG004pU004pU004 | | |
| | pG005pT005 | | |
| | X033A1027p001C007p001A004pA004pC004pA007pU004pU004 | AS | 1464 |
| | pG004pU004pA004pG004pC004pU007pA004pC007pA004pG004 | | |
| | pA004pA004pU004p001C004p001C004 | | |
| 711 | T005p001G005p001U004pA004pG004pC004pU007pA004pC007 | SS | 1450 |
| | pA007pA007pU004pG004pU004pU004pG004pU004pC004pA004 | | |
| | pA005pA005 | | |
| | X033U1027p001U007p001U004pG004pA004pC007pA004pA004 | AS | 1465 |
| | pC004pA004pU004pU004pG004pU007pA004pG007pC004pU004 | | |
| | pA004pC004pA004p001G004p001A004 | | |
| 712 | T005p001G005p001U004pA004pG004pC004pU007pA004pC007 | SS | 1451 |
| | pA007pA007pU004pG004pU004pU004pG004pU004pC004pA004 | | |
| | p001A005p001A005 | | |
| | X033U1027p001U007p001U004pG004pA004pC007pA004pA004 | AS | 1466 |
| | pC004pA004pU004pU004pG004pU007pA004pG007pC004pU004 | | |
| | pA004pC004pA004p001G004p001A004 | | |
| 713 | T005p001G005p001U004pU004pG004pU004pC007pA004pA007 | SS | 1452 |
| | pG007pA007pC004pU004pU004pU004pU004pU004pC004pG004 | | |
| | p001A005p001A005 | | |
| | X033U1027p001U007p001C004pG004pA004pA007pA004pA004 | AS | 1467 |
| | pA004pG004pU004pC004pU004pU007pG004pA007pC004pA004 | | |
| | pA004pC004pA004p001U004p001U004 | | |
| 714 | A005p001C005*p001A004pU004pA004pA004pA007pA004pU00 | SS | 1453 |
| | 7pC007pU007pG004pU004pG004pA004pA004pU004pU004pA00 | | |
| | 4pA005pA005 | | |
| | X033U1027p001U007p001U004pA004pA004pU007pU004pC004 | AS | 1468 |
| | pA004pC004pA004pG004pA004pU007pU004pU007pU004pA004 | | |
| | pU004pG004pU004p001U004p001A004 | | |
| 715 | A005p001C005*p001A004pU004pA004pA004pA007pA004pU00 | SS | 1454 |
| | 7pC007pU007pG004pU004pG004pA004pA004pU004pU004pA00 | | |
| | 4p001A005p001A005 | | |
| | X033U1027p001U007p001U004pA004pA004pU007pU004pC004 | AS | 1469 |
| | pA004pC004pA004pG004pA004pU007pU004pU007pU004pA004 | | |
| | pU004pG004pU004p001U004p001A004 | | |
| 716 | A005p001A005p001G004pG004pA004pC004pU007pA004pA007 | SS | 1455 |
| | pC007pA007pU004pA004pA004pA004pA004pU004pC004pU004 | | |
| | pG005pT005 | | |
| | X033A1027p001C007p001A004pG004pA004pU007pU004pU004 | AS | 1470 |
| | pU004pA004pU004pG004pU004pU007pA004pG007pU004pC004 | | |
| | pC004pU004pU004p001U004p001A004 | | |
| 717 | A005p001A005p001G004pG004pA004pC004pU007pA004pA007 | SS | 1456 |
| | pC007pA007pU004pA004pA004pA004pA004pU004pC004pU004 | | |
| | p001G005p001T005 | | |
| | X033A1027p001C007p001A004pG004pA004pU007pU004pU004 | AS | 1471 |
| | pU004pA004pU004pG004pU004pU007pA004pG007pU004pC004 | | |
| | pC004pU004pU004p001U004p001A004 | | |
| 718 | T005p001A005p001A004pG004pU004pU004pC007pA004pU007 | SS | 1457 |
| | pG007pU007pU004pU004pG004pU004pA004pA004pA004pU004 | | |
| | pT005pA005 | | |
| | X033U1027p001A007p001A004pU004pU004pU007pA004pC004 | AS | 1472 |
| | pA004pA004pA004pC004pA004pU007pG004pA007pA004pC004 | | |
| | pU004pU004pA004p001G004p001A004 | | |
| 720 | T005p001A005p001A004pG004pU004pU004pC007pA004pU007 | SS | 1458 |
| | pG007pU007pU004pU004pG004pU004pA004pA004pA004pU004 | | |
| | p001T005p001A005 | | |
| | X033U1027p001A007p001A004pU004pU004pU007pA004pC004 | AS | 1473 |
| | pA004pA004pA004pC004pA004pU007pG004pA007pA004pC004 | | |
| | pU004pU004pA004p001G004p001A004 | | |
| 721 | T005p001T005p001A004pA004pA004pC004pA007pU004pG007 | SS | 1459 |
| | pC007pU007pA004pA004pA004pU004pA004pG004pU004pU004 | | |
| | pC005*pT005 | | |
| | X033A1027p001G007p001A004pA004pC004pU007pA004pU004 | AS | 1474 |
| | pU004pU004pA004pG004pC004pA007pU004pG007pU004pU004 | | |
| | pU004pA004pA004p001C004p001A004 | | |
| 722 | T005p001T005p001A004pA004pA004pC004pA007pU004pG007 | SS | 1460 |
| | pC007pU007pA004pA004pA004pU004pA004pG004pU004pU004 | | |
| | p001C005*p001T005 | | |
| | X033A1027p001G007p001A004pA004pC004pU007pA004pU004 | AS | 1475 |
| | pU004pU004pA004pG004pC004pA007pU004pG007pU004pU004 | | |
| | pU004pA004pA004p001C004p001A004 | | |
| 723 | T005p001T005p001G004pC004pA004pG004pA007pU004pG007 | SS | 1461 |
| | pC007pU007pA004pG004pG004pU004pG004pU004pU004pC004 | | |
| | pA005pA005 | | |
| | X033U1027p001U007p001G004pA004pA004pC007pA004pC004 | AS | 1476 |
| | pC004pU004pA004pG004pC004pA007pU004pC007pU004pG004 | | |
| | pC004pA004pA004p001A004p001C004 | | |
| 724 | T005p001T005p001G004pC004pA004pG004pA007pU004pG007 | SS | 1462 |
| | pC007pU007pA004pG004pG004pU004pG004pU004pU004pC004 | | |
| | p001A005p001A005 | | |
| | X033U1027p001U007p001G004pA004pA004pC007pA004pC004 | AS | 1477 |
| | pC004pU004pA004pG004pC004pA007pU004pC007pU004pG004 | | |
| | pC004pA004pA004p001A004p001C004 | | |
| 725 | U042p001U042p001G004pC004pA004pG004pA007pU004pG007 | SS | 1481 |
| | pC007pU007pA004pG004pG004pU004pG004pU004pU004pC004 | | |
| | pA042pA042 | | |
| | X033U1027p001U007p001G004pA004pA004pC007pA004pC004 | AS | 1299 |
| | pC004pU004pA004pG004pC004pA007pU004pC007pU004pG004 | | |
| | pC004pA004pA004p001A004p001C004 | | |
| 726 | U042p001U042p001G004pC004pA004pG004pA007pU004pG007 | SS | 1482 |
| | pC007pU007pA004pG004pG004pU004pG004pU004pU004pC004 | | |
| | pA042p001A042 | | |
| | X033U1027p001U007p001G004pA004pA004pC007pA004pC004 | AS | 1299 |
| | pC004pU004pA004pG004pC004pA007pU004pC007pU004pG004 | | |
| | pC004pA004pA004p001A004p001C004 | | |
| 727 | T005p001G005p001U004pA004pG004pC004pU007pA004pC007 | SS | 1451 |
| | pA007pA007pU004pG004pU004pU004pG004pU004pC004pA004 | | |
| | p001A005p001A005 | | |
| | X033U1027p001U007p001U042pG004pA004pC007pA004pA004 | AS | 2600 |
| | pC004pA004pU004pU004pG004pU007pA004pG007pC004pU004 | | |
| | pA004pC004pA004p001G004p001A004 | | |
| 728 | A005p001A005p001G004pG004pA004pC004pU007pA004pA007 | SS | 1456 |
| | pC007pA007pU004pA004pA004pA004pA004pU004pC004pU004 | | |
| | p001G005p001T005 | | |
| | X033A1027p001C007p001A042pG004pA004pU007pU004pU004 | AS | 2601 |
| | pU004pA004pU004pG004pU004pU007pA004pG007pU004pC004 | | |
| | pC004pU004pU004p001U004p001A004 | | |
| 729 | T005p001G005p001U004pU004pG004pU004pC007pA004pA007 | SS | 1452 |
| | pG007pA007pC004pU004pU004pU004pU004pU004pC004pG004 | | |
| | p001A005p001A005 | | |
| | X033U1027p001U007p001C042pG004pA004pA007pA004pA004 | AS | 2602 |
| | pA004pG004pU004pC004pU004pU007pG004pA007pC004pA004 | | |
| | pA004pC004pA004p001U004p001U004 | | |
| 730 | A005p001C005*p001A004pU004pA004pA004pA007pA004pU00 | SS | 1454 |
| | 7pC007pU007pG004pU004pG004pA004pA004pU004pU004pA00 | | |
| | 4p001A005p001A005 | | |
| | X033U1027p001U007p001U042pA004pA004pU007pU004pC004 | AS | 2603 |
| | pA004pC004pA004pG004pA004pU007pU004pU007pU004pA004 | | |
| | pU004pG004pU004p001U004p001A004 | | |
| 731 | T005p001A005p001A004pG004pU004pU004pC007pA004pU007 | SS | 1458 |
| | pG007pU007pU004pU004pG004pU004pA004pA004pA004pU004 | | |
| | p001T005p001A005 | | |
| | X033U1027p001A007p001A042pU004pU004pU007pA004pC004 | AS | 2604 |
| | pA004pA004pA004pC004pA004pU007pG004pA007pA004pC004 | | |
| | pU004pU004pA004p001G004p001A004 | | |
| 732 | T005p001T005p001A004pA004pA004pC004pA007pU004pG007 | SS | 1460 |
| | pC007pU007pA004pA004pA004pU004pA004pG004pU004pU004 | | |
| | p001C005*p001T005 | | |
| | X033A1027p001G007p001A042pA004pC004pU007pA004pU004 | AS | 2605 |
| | pU004pU004pA004pG004pC004pA007pU004pG007pU004pU004 | | |
| | pU004pA004pA004p001C004p001A004 | | |

In Table 3, the nucleotide indicated with “T” is referred to a ribonucleotide having thymidine nucleobase (“ribothymidine”) and “U” is referred to a ribonucleotide having uracil nucleobase (“uridine”). In some embodiments, “T” ribonucleotide (ribothymidine) above may be interchangeably use as “methylated uridine,” “5-methyluridine” or “mU.”

In some embodiments, the sequence in Table 3 may include modified nucleobases. In some embodiments, the sense strand may include one or more nucleotides containing thymine or methylated uracil nucleobase. In some embodiments, the first nucleotide at 5′ end of the sense strand contains the thymine or methylated uracil. In some embodiments, the first nucleotide at 5′ end of the sense strand contains the thymine or methylated uracil. In some embodiments, the sense strand may include one or more nucleotides containing methylate cytosine nucleobase (e.g., 5-methylcytosine or N4-methylcytosine). Example sequences containing modified nucleobases are described in Table 3 above (e.g., C005*: 5-methyl-cytidine/2′ MOE).

In some embodiments, the terminal T at 5′ end of the sense strand in the sequences (SEQ ID Nos: 1294 to 1297, 1448 to 1462, and 1481 to 1482 in Table 3) may not be a part of the HMGCR target mRNA sequence. In some embodiments, the terminal U (uridine with 5′(E)-VP-2′-OMe) at 5′ end of the antisense strand in the sequences (SEQ ID Nos: 1298 to 1301, 1463 to 1477, and 2600 to 2605 in Table 3) may not be a part of the complementary sequence to the HMGCR mRNA target region.

In some embodiments, the dsRNA includes (i) a sense strand having 15 contiguous nucleotides differing by no more than one, two or three nucleotides from the nucleotide sequence selected from SEQ ID NO: 1294 and (ii) an antisense strand forming a duplex with the sense strand of (i) and having 15 contiguous nucleotides differing by no more than one, two or three nucleotides from the nucleotide sequence selected from SEQ ID NO: 1298. In some embodiments, the dsRNA includes (i) a sense strand having 16 contiguous nucleotides differing by no more than one, two or three from the nucleotide sequence selected from SEQ ID NO: 1294 and (ii) an antisense strand forming a duplex with the sense strand of (i) and having 16 contiguous nucleotides differing by no more than one, two or three nucleotides from the nucleotide sequence selected from SEQ ID NO: 1298. In some embodiments, the dsRNA includes (i) a sense strand having 17 contiguous nucleotides differing by no more than one, two or three nucleotides from the nucleotide sequence selected from SEQ ID NO: 1294 and (ii) an antisense strand forming a duplex with the sense strand of (i) and having 17 contiguous nucleotides differing by no more than one, two or three nucleotides from the nucleotide sequence selected from SEQ ID NO: 1298. In some embodiments, the dsRNA includes (i) a sense strand having 18 contiguous nucleotides differing by no more than one, two or three nucleotides from the nucleotide sequence selected from SEQ ID NO: 1294 and (ii) an antisense strand forming a duplex with the sense strand of (i) and having 18 contiguous nucleotides differing by no more than one, two or three nucleotides from the nucleotide sequence selected from SEQ ID NO: 1298. In some embodiments, the dsRNA includes (i) a sense strand having 19 contiguous nucleotides differing by no more than one, two or three nucleotides from the nucleotide sequence selected from SEQ ID NO: 1294 and (ii) an antisense strand forming a duplex with the sense strand of (i) and having 19 contiguous nucleotides differing by no more than one, two or three nucleotides from the nucleotide sequence selected from SEQ ID NO: 1298. In some embodiments, the dsRNA includes (i) a sense strand having 20 contiguous nucleotides differing by no more than one, two or three nucleotides from the nucleotide sequence selected from SEQ ID NO: 1294 and (ii) an antisense strand forming a duplex with the sense strand of (i) and having 20 contiguous nucleotides differing by no more than one, two or three nucleotides from the nucleotide sequence selected from SEQ ID NO: 1298. In some embodiments, the dsRNA includes (i) a sense strand having 21 contiguous nucleotides differing by no more than one, two or three nucleotides from the nucleotide sequence selected from SEQ ID NO: 1294 and (ii) an antisense strand forming a duplex with the sense strand of (i) and having 21 contiguous nucleotides differing by no more than one, two or three nucleotides from the nucleotide sequence selected from SEQ ID NO: 1298.

In some embodiments, the dsRNA includes (i) a sense strand having 15 contiguous nucleotides differing by no more than one, two or three nucleotides from the nucleotide sequence selected from SEQ ID NO: 1295 and (ii) an antisense strand forming a duplex with the sense strand of (i) and having 15 contiguous nucleotides differing by no more than one, two or three nucleotides from the nucleotide sequence selected from SEQ ID NO: 1299. In some embodiments, the dsRNA includes (i) a sense strand having 16 contiguous nucleotides differing by no more than one, two or three from the nucleotide sequence selected from SEQ ID NO: 1295 and (ii) an antisense strand forming a duplex with the sense strand of (i) and having 16 contiguous nucleotides differing by no more than one, two or three nucleotides from the nucleotide sequence selected from SEQ ID NO: 1299. In some embodiments, the dsRNA includes (i) a sense strand having 17 contiguous nucleotides differing by no more than one, two or three nucleotides from the nucleotide sequence selected from SEQ ID NO: 1295 and (ii) an antisense strand forming a duplex with the sense strand of (i) and having 17 contiguous nucleotides differing by no more than one, two or three nucleotides from the nucleotide sequence selected from SEQ ID NO: 1299. In some embodiments, the dsRNA includes (i) a sense strand having 18 contiguous nucleotides differing by no more than one, two or three nucleotides from the nucleotide sequence selected from SEQ ID NO: 1295 and (ii) an antisense strand forming a duplex with the sense strand of (i) and having 18 contiguous nucleotides differing by no more than one, two or three nucleotides from the nucleotide sequence selected from SEQ ID NO: 1299. In some embodiments, the dsRNA includes (i) a sense strand having 19 contiguous nucleotides differing by no more than one, two or three nucleotides from the nucleotide sequence selected from SEQ ID NO: 1295 and (ii) an antisense strand forming a duplex with the sense strand of (i) and having 19 contiguous nucleotides differing by no more than one, two or three nucleotides from the nucleotide sequence selected from SEQ ID NO: 1299. In some embodiments, the dsRNA includes (i) a sense strand having 20 contiguous nucleotides differing by no more than one, two or three nucleotides from the nucleotide sequence selected from SEQ ID NO: 1295 and (ii) an antisense strand forming a duplex with the sense strand of (i) and having 20 contiguous nucleotides differing by no more than one, two or three nucleotides from the nucleotide sequence selected from SEQ ID NO: 1299. In some embodiments, the dsRNA includes (i) a sense strand having 21 contiguous nucleotides differing by no more than one, two or three nucleotides from the nucleotide sequence selected from SEQ ID NO: 1295 and (ii) an antisense strand forming a duplex with the sense strand of (i) and having 21 contiguous nucleotides differing by no more than one, two or three nucleotides from the nucleotide sequence selected from SEQ ID NO: 1299.

In some embodiments, the dsRNA includes (i) a sense strand having 15 contiguous nucleotides differing by no more than one, two or three nucleotides from the nucleotide sequence selected from SEQ ID NO: 1296 and (ii) an antisense strand forming a duplex with the sense strand of (i) and having 15 contiguous nucleotides differing by no more than one, two or three nucleotides from the nucleotide sequence selected from SEQ ID NO: 1300. In some embodiments, the dsRNA includes (i) a sense strand having 16 contiguous nucleotides differing by no more than one, two or three from the nucleotide sequence selected from SEQ ID NO: 1296 and (ii) an antisense strand forming a duplex with the sense strand of (i) and having 16 contiguous nucleotides differing by no more than one, two or three nucleotides from the nucleotide sequence selected from SEQ ID NO: 1300. In some embodiments, the dsRNA includes (i) a sense strand having 17 contiguous nucleotides differing by no more than one, two or three nucleotides from the nucleotide sequence selected from SEQ ID NO: 1296 and (ii) an antisense strand forming a duplex with the sense strand of (i) and having 17 contiguous nucleotides differing by no more than one, two or three nucleotides from the nucleotide sequence selected from SEQ ID NO: 1300. In some embodiments, the dsRNA includes (i) a sense strand having 18 contiguous nucleotides differing by no more than one, two or three nucleotides from the nucleotide sequence selected from SEQ ID NO: 1296 and (ii) an antisense strand forming a duplex with the sense strand of (i) and having 18 contiguous nucleotides differing by no more than one, two or three nucleotides from the nucleotide sequence selected from SEQ ID NO: 1300. In some embodiments, the dsRNA includes (i) a sense strand having 19 contiguous nucleotides differing by no more than one, two or three nucleotides from the nucleotide sequence selected from SEQ ID NO: 1296 and (ii) an antisense strand forming a duplex with the sense strand of (i) and having 19 contiguous nucleotides differing by no more than one, two or three nucleotides from the nucleotide sequence selected from SEQ ID NO: 1300. In some embodiments, the dsRNA includes (i) a sense strand having 20 contiguous nucleotides differing by no more than one, two or three nucleotides from the nucleotide sequence selected from SEQ ID NO: 1296 and (ii) an antisense strand forming a duplex with the sense strand of (i) and having 20 contiguous nucleotides differing by no more than one, two or three nucleotides from the nucleotide sequence selected from SEQ ID NO: 1300. In some embodiments, the dsRNA includes (i) a sense strand having 21 contiguous nucleotides differing by no more than one, two or three nucleotides from the nucleotide sequence selected from SEQ ID NO: 1296 and (ii) an antisense strand forming a duplex with the sense strand of (i) and having 21 contiguous nucleotides differing by no more than one, two or three nucleotides from the nucleotide sequence selected from SEQ ID NO: 1300.

In some embodiments, the dsRNA includes (i) a sense strand having 15 contiguous nucleotides differing by no more than one, two or three nucleotides from the nucleotide sequence selected from SEQ ID NO: 1297 and (ii) an antisense strand forming a duplex with the sense strand of (i) and having 15 contiguous nucleotides differing by no more than one, two or three nucleotides from the nucleotide sequence selected from SEQ ID NO: 1301. In some embodiments, the dsRNA includes (i) a sense strand having 16 contiguous nucleotides differing by no more than one, two or three from the nucleotide sequence selected from SEQ ID NO: 1297 and (ii) an antisense strand forming a duplex with the sense strand of (i) and having 16 contiguous nucleotides differing by no more than one, two or three nucleotides from the nucleotide sequence selected from SEQ ID NO: 1301. In some embodiments, the dsRNA includes (i) a sense strand having 17 contiguous nucleotides differing by no more than one, two or three nucleotides from the nucleotide sequence selected from SEQ ID NO: 1297 and (ii) an antisense strand forming a duplex with the sense strand of (i) and having 17 contiguous nucleotides differing by no more than one, two or three nucleotides from the nucleotide sequence selected from SEQ ID NO: 1301. In some embodiments, the dsRNA includes (i) a sense strand having 18 contiguous nucleotides differing by no more than one, two or three nucleotides from the nucleotide sequence selected from SEQ ID NO: 1297 and (ii) an antisense strand forming a duplex with the sense strand of (i) and having 18 contiguous nucleotides differing by no more than one, two or three nucleotides from the nucleotide sequence selected from SEQ ID NO: 1301. In some embodiments, the dsRNA includes (i) a sense strand having 19 contiguous nucleotides differing by no more than one, two or three nucleotides from the nucleotide sequence selected from SEQ ID NO: 1297 and (ii) an antisense strand forming a duplex with the sense strand of (i) and having 19 contiguous nucleotides differing by no more than one, two or three nucleotides from the nucleotide sequence selected from SEQ ID NO: 1301. In some embodiments, the dsRNA includes (i) a sense strand having 20 contiguous nucleotides differing by no more than one, two or three nucleotides from the nucleotide sequence selected from SEQ ID NO: 1297 and (ii) an antisense strand forming a duplex with the sense strand of (i) and having 20 contiguous nucleotides differing by no more than one, two or three nucleotides from the nucleotide sequence selected from SEQ ID NO: 1301. In some embodiments, the dsRNA includes (i) a sense strand having 21 contiguous nucleotides differing by no more than one, two or three nucleotides from the nucleotide sequence selected from SEQ ID NO: 1297 and (ii) an antisense strand forming a duplex with the sense strand of (i) and having 21 contiguous nucleotides differing by no more than one, two or three nucleotides from the nucleotide sequence selected from SEQ ID NO: 1301.

In some embodiments, the dsRNA includes (i) a sense strand having 15 contiguous nucleotides differing by no more than one, two or three nucleotides from the nucleotide sequence selected from SEQ ID NO: 1448 and (ii) an antisense strand forming a duplex with the sense strand of (i) and having 15 contiguous nucleotides differing by no more than one, two or three nucleotides from the nucleotide sequence selected from SEQ ID NO: 1463. In some embodiments, the dsRNA includes (i) a sense strand having 16 contiguous nucleotides differing by no more than one, two or three from the nucleotide sequence selected from SEQ ID NO: 1448 and (ii) an antisense strand forming a duplex with the sense strand of (i) and having 16 contiguous nucleotides differing by no more than one, two or three nucleotides from the nucleotide sequence selected from SEQ ID NO: 1463. In some embodiments, the dsRNA includes (i) a sense strand having 17 contiguous nucleotides differing by no more than one, two or three nucleotides from the nucleotide sequence selected from SEQ ID NO: 1448 and (ii) an antisense strand forming a duplex with the sense strand of (i) and having 17 contiguous nucleotides differing by no more than one, two or three nucleotides from the nucleotide sequence selected from SEQ ID NO: 1463. In some embodiments, the dsRNA includes (i) a sense strand having 18 contiguous nucleotides differing by no more than one, two or three nucleotides from the nucleotide sequence selected from SEQ ID NO: 1448 and (ii) an antisense strand forming a duplex with the sense strand of (i) and having 18 contiguous nucleotides differing by no more than one, two or three nucleotides from the nucleotide sequence selected from SEQ ID NO: 1463. In some embodiments, the dsRNA includes (i) a sense strand having 19 contiguous nucleotides differing by no more than one, two or three nucleotides from the nucleotide sequence selected from SEQ ID NO: 1448 and (ii) an antisense strand forming a duplex with the sense strand of (i) and having 19 contiguous nucleotides differing by no more than one, two or three nucleotides from the nucleotide sequence selected from SEQ ID NO: 1463. In some embodiments, the dsRNA includes (i) a sense strand having 20 contiguous nucleotides differing by no more than one, two or three nucleotides from the nucleotide sequence selected from SEQ ID NO: 1448 and (ii) an antisense strand forming a duplex with the sense strand of (i) and having 20 contiguous nucleotides differing by no more than one, two or three nucleotides from the nucleotide sequence selected from SEQ ID NO: 1463. In some embodiments, the dsRNA includes (i) a sense strand having 21 contiguous nucleotides differing by no more than one, two or three nucleotides from the nucleotide sequence selected from SEQ ID NO: 1448 and (ii) an antisense strand forming a duplex with the sense strand of (i) and having 21 contiguous nucleotides differing by no more than one, two or three nucleotides from the nucleotide sequence selected from SEQ ID NO: 1463.

In some embodiments, the dsRNA includes (i) a sense strand having 15 contiguous nucleotides differing by no more than one, two or three nucleotides from the nucleotide sequence selected from SEQ ID NO: 1449 and (ii) an antisense strand forming a duplex with the sense strand of (i) and having 15 contiguous nucleotides differing by no more than one, two or three nucleotides from the nucleotide sequence selected from SEQ ID NO: 1464. In some embodiments, the dsRNA includes (i) a sense strand having 16 contiguous nucleotides differing by no more than one, two or three from the nucleotide sequence selected from SEQ ID NO: 1449 and (ii) an antisense strand forming a duplex with the sense strand of (i) and having 16 contiguous nucleotides differing by no more than one, two or three nucleotides from the nucleotide sequence selected from SEQ ID NO: 1464. In some embodiments, the dsRNA includes (i) a sense strand having 17 contiguous nucleotides differing by no more than one, two or three nucleotides from the nucleotide sequence selected from SEQ ID NO: 1449 and (ii) an antisense strand forming a duplex with the sense strand of (i) and having 17 contiguous nucleotides differing by no more than one, two or three nucleotides from the nucleotide sequence selected from SEQ ID NO: 1464. In some embodiments, the dsRNA includes (i) a sense strand having 18 contiguous nucleotides differing by no more than one, two or three nucleotides from the nucleotide sequence selected from SEQ ID NO: 1449 and (ii) an antisense strand forming a duplex with the sense strand of (i) and having 18 contiguous nucleotides differing by no more than one, two or three nucleotides from the nucleotide sequence selected from SEQ ID NO: 1464. In some embodiments, the dsRNA includes (i) a sense strand having 19 contiguous nucleotides differing by no more than one, two or three nucleotides from the nucleotide sequence selected from SEQ ID NO: 1449 and (ii) an antisense strand forming a duplex with the sense strand of (i) and having 19 contiguous nucleotides differing by no more than one, two or three nucleotides from the nucleotide sequence selected from SEQ ID NO: 1464. In some embodiments, the dsRNA includes (i) a sense strand having 20 contiguous nucleotides differing by no more than one, two or three nucleotides from the nucleotide sequence selected from SEQ ID NO: 1449 and (ii) an antisense strand forming a duplex with the sense strand of (i) and having 20 contiguous nucleotides differing by no more than one, two or three nucleotides from the nucleotide sequence selected from SEQ ID NO: 1464. In some embodiments, the dsRNA includes (i) a sense strand having 21 contiguous nucleotides differing by no more than one, two or three nucleotides from the nucleotide sequence selected from SEQ ID NO: 1449 and (ii) an antisense strand forming a duplex with the sense strand of (i) and having 21 contiguous nucleotides differing by no more than one, two or three nucleotides from the nucleotide sequence selected from SEQ ID NO: 1464.

In some embodiments, the dsRNA includes (i) a sense strand having 15 contiguous nucleotides differing by no more than one, two or three nucleotides from the nucleotide sequence selected from SEQ ID NO: 1450 and (ii) an antisense strand forming a duplex with the sense strand of (i) and having 15 contiguous nucleotides differing by no more than one, two or three nucleotides from the nucleotide sequence selected from SEQ ID NO: 1465. In some embodiments, the dsRNA includes (i) a sense strand having 16 contiguous nucleotides differing by no more than one, two or three from the nucleotide sequence selected from SEQ ID NO: 1450 and (ii) an antisense strand forming a duplex with the sense strand of (i) and having 16 contiguous nucleotides differing by no more than one, two or three nucleotides from the nucleotide sequence selected from SEQ ID NO: 1465. In some embodiments, the dsRNA includes (i) a sense strand having 17 contiguous nucleotides differing by no more than one, two or three nucleotides from the nucleotide sequence selected from SEQ ID NO: 1450 and (ii) an antisense strand forming a duplex with the sense strand of (i) and having 17 contiguous nucleotides differing by no more than one, two or three nucleotides from the nucleotide sequence selected from SEQ ID NO: 1465. In some embodiments, the dsRNA includes (i) a sense strand having 18 contiguous nucleotides differing by no more than one, two or three nucleotides from the nucleotide sequence selected from SEQ ID NO: 1450 and (ii) an antisense strand forming a duplex with the sense strand of (i) and having 18 contiguous nucleotides differing by no more than one, two or three nucleotides from the nucleotide sequence selected from SEQ ID NO: 1465. In some embodiments, the dsRNA includes (i) a sense strand having 19 contiguous nucleotides differing by no more than one, two or three nucleotides from the nucleotide sequence selected from SEQ ID NO: 1450 and (ii) an antisense strand forming a duplex with the sense strand of (i) and having 19 contiguous nucleotides differing by no more than one, two or three nucleotides from the nucleotide sequence selected from SEQ ID NO: 1465. In some embodiments, the dsRNA includes (i) a sense strand having 20 contiguous nucleotides differing by no more than one, two or three nucleotides from the nucleotide sequence selected from SEQ ID NO: 1450 and (ii) an antisense strand forming a duplex with the sense strand of (i) and having 20 contiguous nucleotides differing by no more than one, two or three nucleotides from the nucleotide sequence selected from SEQ ID NO: 1465. In some embodiments, the dsRNA includes (i) a sense strand having 21 contiguous nucleotides differing by no more than one, two or three nucleotides from the nucleotide sequence selected from SEQ ID NO: 1450 and (ii) an antisense strand forming a duplex with the sense strand of (i) and having 21 contiguous nucleotides differing by no more than one, two or three nucleotides from the nucleotide sequence selected from SEQ ID NO: 1465.

In some embodiments, the dsRNA includes (i) a sense strand having 15 contiguous nucleotides differing by no more than one, two or three nucleotides from the nucleotide sequence selected from SEQ ID NO: 1451 and (ii) an antisense strand forming a duplex with the sense strand of (i) and having 15 contiguous nucleotides differing by no more than one, two or three nucleotides from the nucleotide sequence selected from SEQ ID NO: 1466. In some embodiments, the dsRNA includes (i) a sense strand having 16 contiguous nucleotides differing by no more than one, two or three from the nucleotide sequence selected from SEQ ID NO: 1451 and (ii) an antisense strand forming a duplex with the sense strand of (i) and having 16 contiguous nucleotides differing by no more than one, two or three nucleotides from the nucleotide sequence selected from SEQ ID NO: 1466. In some embodiments, the dsRNA includes (i) a sense strand having 17 contiguous nucleotides differing by no more than one, two or three nucleotides from the nucleotide sequence selected from SEQ ID NO: 1451 and (ii) an antisense strand forming a duplex with the sense strand of (i) and having 17 contiguous nucleotides differing by no more than one, two or three nucleotides from the nucleotide sequence selected from SEQ ID NO: 1466. In some embodiments, the dsRNA includes (i) a sense strand having 18 contiguous nucleotides differing by no more than one, two or three nucleotides from the nucleotide sequence selected from SEQ ID NO: 1451 and (ii) an antisense strand forming a duplex with the sense strand of (i) and having 18 contiguous nucleotides differing by no more than one, two or three nucleotides from the nucleotide sequence selected from SEQ ID NO: 1466. In some embodiments, the dsRNA includes (i) a sense strand having 19 contiguous nucleotides differing by no more than one, two or three nucleotides from the nucleotide sequence selected from SEQ ID NO: 1451 and (ii) an antisense strand forming a duplex with the sense strand of (i) and having 19 contiguous nucleotides differing by no more than one, two or three nucleotides from the nucleotide sequence selected from SEQ ID NO: 1466. In some embodiments, the dsRNA includes (i) a sense strand having 20 contiguous nucleotides differing by no more than one, two or three nucleotides from the nucleotide sequence selected from SEQ ID NO: 1451 and (ii) an antisense strand forming a duplex with the sense strand of (i) and having 20 contiguous nucleotides differing by no more than one, two or three nucleotides from the nucleotide sequence selected from SEQ ID NO: 1466. In some embodiments, the dsRNA includes (i) a sense strand having 21 contiguous nucleotides differing by no more than one, two or three nucleotides from the nucleotide sequence selected from SEQ ID NO: 1451 and (ii) an antisense strand forming a duplex with the sense strand of (i) and having 21 contiguous nucleotides differing by no more than one, two or three nucleotides from the nucleotide sequence selected from SEQ ID NO: 1466.

In some embodiments, the dsRNA includes (i) a sense strand having 15 contiguous nucleotides differing by no more than one, two or three nucleotides from the nucleotide sequence selected from SEQ ID NO: 1452 and (ii) an antisense strand forming a duplex with the sense strand of (i) and having 15 contiguous nucleotides differing by no more than one, two or three nucleotides from the nucleotide sequence selected from SEQ ID NO: 1467. In some embodiments, the dsRNA includes (i) a sense strand having 16 contiguous nucleotides differing by no more than one, two or three from the nucleotide sequence selected from SEQ ID NO: 1452 and (ii) an antisense strand forming a duplex with the sense strand of (i) and having 16 contiguous nucleotides differing by no more than one, two or three nucleotides from the nucleotide sequence selected from SEQ ID NO: 1467. In some embodiments, the dsRNA includes (i) a sense strand having 17 contiguous nucleotides differing by no more than one, two or three nucleotides from the nucleotide sequence selected from SEQ ID NO: 1452 and (ii) an antisense strand forming a duplex with the sense strand of (i) and having 17 contiguous nucleotides differing by no more than one, two or three nucleotides from the nucleotide sequence selected from SEQ ID NO: 1467. In some embodiments, the dsRNA includes (i) a sense strand having 18 contiguous nucleotides differing by no more than one, two or three nucleotides from the nucleotide sequence selected from SEQ ID NO: 1452 and (ii) an antisense strand forming a duplex with the sense strand of (i) and having 18 contiguous nucleotides differing by no more than one, two or three nucleotides from the nucleotide sequence selected from SEQ ID NO: 1467. In some embodiments, the dsRNA includes (i) a sense strand having 19 contiguous nucleotides differing by no more than one, two or three nucleotides from the nucleotide sequence selected from SEQ ID NO: 1452 and (ii) an antisense strand forming a duplex with the sense strand of (i) and having 19 contiguous nucleotides differing by no more than one, two or three nucleotides from the nucleotide sequence selected from SEQ ID NO: 1467. In some embodiments, the dsRNA includes (i) a sense strand having 20 contiguous nucleotides differing by no more than one, two or three nucleotides from the nucleotide sequence selected from SEQ ID NO: 1452 and (ii) an antisense strand forming a duplex with the sense strand of (i) and having 20 contiguous nucleotides differing by no more than one, two or three nucleotides from the nucleotide sequence selected from SEQ ID NO: 1467. In some embodiments, the dsRNA includes (i) a sense strand having 21 contiguous nucleotides differing by no more than one, two or three nucleotides from the nucleotide sequence selected from SEQ ID NO: 1452 and (ii) an antisense strand forming a duplex with the sense strand of (i) and having 21 contiguous nucleotides differing by no more than one, two or three nucleotides from the nucleotide sequence selected from SEQ ID NO: 1467.

In some embodiments, the dsRNA includes (i) a sense strand having 15 contiguous nucleotides differing by no more than one, two or three nucleotides from the nucleotide sequence selected from SEQ ID NO: 1453 and (ii) an antisense strand forming a duplex with the sense strand of (i) and having 15 contiguous nucleotides differing by no more than one, two or three nucleotides from the nucleotide sequence selected from SEQ ID NO: 1468. In some embodiments, the dsRNA includes (i) a sense strand having 16 contiguous nucleotides differing by no more than one, two or three from the nucleotide sequence selected from SEQ ID NO: 1453 and (ii) an antisense strand forming a duplex with the sense strand of (i) and having 16 contiguous nucleotides differing by no more than one, two or three nucleotides from the nucleotide sequence selected from SEQ ID NO: 1468. In some embodiments, the dsRNA includes (i) a sense strand having 17 contiguous nucleotides differing by no more than one, two or three nucleotides from the nucleotide sequence selected from SEQ ID NO: 1453 and (ii) an antisense strand forming a duplex with the sense strand of (i) and having 17 contiguous nucleotides differing by no more than one, two or three nucleotides from the nucleotide sequence selected from SEQ ID NO: 1468. In some embodiments, the dsRNA includes (i) a sense strand having 18 contiguous nucleotides differing by no more than one, two or three nucleotides from the nucleotide sequence selected from SEQ ID NO: 1453 and (ii) an antisense strand forming a duplex with the sense strand of (i) and having 18 contiguous nucleotides differing by no more than one, two or three nucleotides from the nucleotide sequence selected from SEQ ID NO: 1468. In some embodiments, the dsRNA includes (i) a sense strand having 19 contiguous nucleotides differing by no more than one, two or three nucleotides from the nucleotide sequence selected from SEQ ID NO: 1453 and (ii) an antisense strand forming a duplex with the sense strand of (i) and having 19 contiguous nucleotides differing by no more than one, two or three nucleotides from the nucleotide sequence selected from SEQ ID NO: 1468. In some embodiments, the dsRNA includes (i) a sense strand having 20 contiguous nucleotides differing by no more than one, two or three nucleotides from the nucleotide sequence selected from SEQ ID NO: 1453 and (ii) an antisense strand forming a duplex with the sense strand of (i) and having 20 contiguous nucleotides differing by no more than one, two or three nucleotides from the nucleotide sequence selected from SEQ ID NO: 1468. In some embodiments, the dsRNA includes (i) a sense strand having 21 contiguous nucleotides differing by no more than one, two or three nucleotides from the nucleotide sequence selected from SEQ ID NO: 1453 and (ii) an antisense strand forming a duplex with the sense strand of (i) and having 21 contiguous nucleotides differing by no more than one, two or three nucleotides from the nucleotide sequence selected from SEQ ID NO: 1468.

In some embodiments, the dsRNA includes (i) a sense strand having 15 contiguous nucleotides differing by no more than one, two or three nucleotides from the nucleotide sequence selected from SEQ ID NO: 1454 and (ii) an antisense strand forming a duplex with the sense strand of (i) and having 15 contiguous nucleotides differing by no more than one, two or three nucleotides from the nucleotide sequence selected from SEQ ID NO: 1469. In some embodiments, the dsRNA includes (i) a sense strand having 16 contiguous nucleotides differing by no more than one, two or three from the nucleotide sequence selected from SEQ ID NO: 1454 and (ii) an antisense strand forming a duplex with the sense strand of (i) and having 16 contiguous nucleotides differing by no more than one, two or three nucleotides from the nucleotide sequence selected from SEQ ID NO: 1469. In some embodiments, the dsRNA includes (i) a sense strand having 17 contiguous nucleotides differing by no more than one, two or three nucleotides from the nucleotide sequence selected from SEQ ID NO: 1454 and (ii) an antisense strand forming a duplex with the sense strand of (i) and having 17 contiguous nucleotides differing by no more than one, two or three nucleotides from the nucleotide sequence selected from SEQ ID NO: 1469. In some embodiments, the dsRNA includes (i) a sense strand having 18 contiguous nucleotides differing by no more than one, two or three nucleotides from the nucleotide sequence selected from SEQ ID NO: 1454 and (ii) an antisense strand forming a duplex with the sense strand of (i) and having 18 contiguous nucleotides differing by no more than one, two or three nucleotides from the nucleotide sequence selected from SEQ ID NO: 1469. In some embodiments, the dsRNA includes (i) a sense strand having 19 contiguous nucleotides differing by no more than one, two or three nucleotides from the nucleotide sequence selected from SEQ ID NO: 1454 and (ii) an antisense strand forming a duplex with the sense strand of (i) and having 19 contiguous nucleotides differing by no more than one, two or three nucleotides from the nucleotide sequence selected from SEQ ID NO: 1469. In some embodiments, the dsRNA includes (i) a sense strand having 20 contiguous nucleotides differing by no more than one, two or three nucleotides from the nucleotide sequence selected from SEQ ID NO: 1454 and (ii) an antisense strand forming a duplex with the sense strand of (i) and having 20 contiguous nucleotides differing by no more than one, two or three nucleotides from the nucleotide sequence selected from SEQ ID NO: 1469. In some embodiments, the dsRNA includes (i) a sense strand having 21 contiguous nucleotides differing by no more than one, two or three nucleotides from the nucleotide sequence selected from SEQ ID NO: 1454 and (ii) an antisense strand forming a duplex with the sense strand of (i) and having 21 contiguous nucleotides differing by no more than one, two or three nucleotides from the nucleotide sequence selected from SEQ ID NO: 1469.

In some embodiments, the dsRNA includes (i) a sense strand having 15 contiguous nucleotides differing by no more than one, two or three nucleotides from the nucleotide sequence selected from SEQ ID NO: 1455 and (ii) an antisense strand forming a duplex with the sense strand of (i) and having 15 contiguous nucleotides differing by no more than one, two or three nucleotides from the nucleotide sequence selected from SEQ ID NO: 1470. In some embodiments, the dsRNA includes (i) a sense strand having 16 contiguous nucleotides differing by no more than one, two or three from the nucleotide sequence selected from SEQ ID NO: 1455 and (ii) an antisense strand forming a duplex with the sense strand of (i) and having 16 contiguous nucleotides differing by no more than one, two or three nucleotides from the nucleotide sequence selected from SEQ ID NO: 1470. In some embodiments, the dsRNA includes (i) a sense strand having 17 contiguous nucleotides differing by no more than one, two or three nucleotides from the nucleotide sequence selected from SEQ ID NO: 1455 and (ii) an antisense strand forming a duplex with the sense strand of (i) and having 17 contiguous nucleotides differing by no more than one, two or three nucleotides from the nucleotide sequence selected from SEQ ID NO: 1470. In some embodiments, the dsRNA includes (i) a sense strand having 18 contiguous nucleotides differing by no more than one, two or three nucleotides from the nucleotide sequence selected from SEQ ID NO: 1455 and (ii) an antisense strand forming a duplex with the sense strand of (i) and having 18 contiguous nucleotides differing by no more than one, two or three nucleotides from the nucleotide sequence selected from SEQ ID NO: 1470. In some embodiments, the dsRNA includes (i) a sense strand having 19 contiguous nucleotides differing by no more than one, two or three nucleotides from the nucleotide sequence selected from SEQ ID NO: 1455 and (ii) an antisense strand forming a duplex with the sense strand of (i) and having 19 contiguous nucleotides differing by no more than one, two or three nucleotides from the nucleotide sequence selected from SEQ ID NO: 1470. In some embodiments, the dsRNA includes (i) a sense strand having 20 contiguous nucleotides differing by no more than one, two or three nucleotides from the nucleotide sequence selected from SEQ ID NO: 1455 and (ii) an antisense strand forming a duplex with the sense strand of (i) and having 20 contiguous nucleotides differing by no more than one, two or three nucleotides from the nucleotide sequence selected from SEQ ID NO: 1470. In some embodiments, the dsRNA includes (i) a sense strand having 21 contiguous nucleotides differing by no more than one, two or three nucleotides from the nucleotide sequence selected from SEQ ID NO: 1455 and (ii) an antisense strand forming a duplex with the sense strand of (i) and having 21 contiguous nucleotides differing by no more than one, two or three nucleotides from the nucleotide sequence selected from SEQ ID NO: 1470.

In some embodiments, the dsRNA includes (i) a sense strand having 15 contiguous nucleotides differing by no more than one, two or three nucleotides from the nucleotide sequence selected from SEQ ID NO: 1456 and (ii) an antisense strand forming a duplex with the sense strand of (i) and having 15 contiguous nucleotides differing by no more than one, two or three nucleotides from the nucleotide sequence selected from SEQ ID NO: 1471. In some embodiments, the dsRNA includes (i) a sense strand having 16 contiguous nucleotides differing by no more than one, two or three from the nucleotide sequence selected from SEQ ID NO: 1456 and (ii) an antisense strand forming a duplex with the sense strand of (i) and having 16 contiguous nucleotides differing by no more than one, two or three nucleotides from the nucleotide sequence selected from SEQ ID NO: 1471. In some embodiments, the dsRNA includes (i) a sense strand having 17 contiguous nucleotides differing by no more than one, two or three nucleotides from the nucleotide sequence selected from SEQ ID NO: 1456 and (ii) an antisense strand forming a duplex with the sense strand of (i) and having 17 contiguous nucleotides differing by no more than one, two or three nucleotides from the nucleotide sequence selected from SEQ ID NO: 1471. In some embodiments, the dsRNA includes (i) a sense strand having 18 contiguous nucleotides differing by no more than one, two or three nucleotides from the nucleotide sequence selected from SEQ ID NO: 1456 and (ii) an antisense strand forming a duplex with the sense strand of (i) and having 18 contiguous nucleotides differing by no more than one, two or three nucleotides from the nucleotide sequence selected from SEQ ID NO: 1471. In some embodiments, the dsRNA includes (i) a sense strand having 19 contiguous nucleotides differing by no more than one, two or three nucleotides from the nucleotide sequence selected from SEQ ID NO: 1456 and (ii) an antisense strand forming a duplex with the sense strand of (i) and having 19 contiguous nucleotides differing by no more than one, two or three nucleotides from the nucleotide sequence selected from SEQ ID NO: 1471. In some embodiments, the dsRNA includes (i) a sense strand having 20 contiguous nucleotides differing by no more than one, two or three nucleotides from the nucleotide sequence selected from SEQ ID NO: 1456 and (ii) an antisense strand forming a duplex with the sense strand of (i) and having 20 contiguous nucleotides differing by no more than one, two or three nucleotides from the nucleotide sequence selected from SEQ ID NO: 1471. In some embodiments, the dsRNA includes (i) a sense strand having 21 contiguous nucleotides differing by no more than one, two or three nucleotides from the nucleotide sequence selected from SEQ ID NO: 1456 and (ii) an antisense strand forming a duplex with the sense strand of (i) and having 21 contiguous nucleotides differing by no more than one, two or three nucleotides from the nucleotide sequence selected from SEQ ID NO: 1471.

In some embodiments, the dsRNA includes (i) a sense strand having 15 contiguous nucleotides differing by no more than one, two or three nucleotides from the nucleotide sequence selected from SEQ ID NO: 1457 and (ii) an antisense strand forming a duplex with the sense strand of (i) and having 15 contiguous nucleotides differing by no more than one, two or three nucleotides from the nucleotide sequence selected from SEQ ID NO: 1472. In some embodiments, the dsRNA includes (i) a sense strand having 16 contiguous nucleotides differing by no more than one, two or three from the nucleotide sequence selected from SEQ ID NO: 1457 and (ii) an antisense strand forming a duplex with the sense strand of (i) and having 16 contiguous nucleotides differing by no more than one, two or three nucleotides from the nucleotide sequence selected from SEQ ID NO: 1472. In some embodiments, the dsRNA includes (i) a sense strand having 17 contiguous nucleotides differing by no more than one, two or three nucleotides from the nucleotide sequence selected from SEQ ID NO: 1457 and (ii) an antisense strand forming a duplex with the sense strand of (i) and having 17 contiguous nucleotides differing by no more than one, two or three nucleotides from the nucleotide sequence selected from SEQ ID NO: 1472. In some embodiments, the dsRNA includes (i) a sense strand having 18 contiguous nucleotides differing by no more than one, two or three nucleotides from the nucleotide sequence selected from SEQ ID NO: 1457 and (ii) an antisense strand forming a duplex with the sense strand of (i) and having 18 contiguous nucleotides differing by no more than one, two or three nucleotides from the nucleotide sequence selected from SEQ ID NO: 1472. In some embodiments, the dsRNA includes (i) a sense strand having 19 contiguous nucleotides differing by no more than one, two or three nucleotides from the nucleotide sequence selected from SEQ ID NO: 1457 and (ii) an antisense strand forming a duplex with the sense strand of (i) and having 19 contiguous nucleotides differing by no more than one, two or three nucleotides from the nucleotide sequence selected from SEQ ID NO: 1472. In some embodiments, the dsRNA includes (i) a sense strand having 20 contiguous nucleotides differing by no more than one, two or three nucleotides from the nucleotide sequence selected from SEQ ID NO: 1457 and (ii) an antisense strand forming a duplex with the sense strand of (i) and having 20 contiguous nucleotides differing by no more than one, two or three nucleotides from the nucleotide sequence selected from SEQ ID NO: 1472. In some embodiments, the dsRNA includes (i) a sense strand having 21 contiguous nucleotides differing by no more than one, two or three nucleotides from the nucleotide sequence selected from SEQ ID NO: 1457 and (ii) an antisense strand forming a duplex with the sense strand of (i) and having 21 contiguous nucleotides differing by no more than one, two or three nucleotides from the nucleotide sequence selected from SEQ ID NO: 1472.

In some embodiments, the dsRNA includes (i) a sense strand having 15 contiguous nucleotides differing by no more than one, two or three nucleotides from the nucleotide sequence selected from SEQ ID NO: 1458 and (ii) an antisense strand forming a duplex with the sense strand of (i) and having 15 contiguous nucleotides differing by no more than one, two or three nucleotides from the nucleotide sequence selected from SEQ ID NO: 1473. In some embodiments, the dsRNA includes (i) a sense strand having 16 contiguous nucleotides differing by no more than one, two or three from the nucleotide sequence selected from SEQ ID NO: 1458 and (ii) an antisense strand forming a duplex with the sense strand of (i) and having 16 contiguous nucleotides differing by no more than one, two or three nucleotides from the nucleotide sequence selected from SEQ ID NO: 1473. In some embodiments, the dsRNA includes (i) a sense strand having 17 contiguous nucleotides differing by no more than one, two or three nucleotides from the nucleotide sequence selected from SEQ ID NO: 1458 and (ii) an antisense strand forming a duplex with the sense strand of (i) and having 17 contiguous nucleotides differing by no more than one, two or three nucleotides from the nucleotide sequence selected from SEQ ID NO: 1473. In some embodiments, the dsRNA includes (i) a sense strand having 18 contiguous nucleotides differing by no more than one, two or three nucleotides from the nucleotide sequence selected from SEQ ID NO: 1458 and (ii) an antisense strand forming a duplex with the sense strand of (i) and having 18 contiguous nucleotides differing by no more than one, two or three nucleotides from the nucleotide sequence selected from SEQ ID NO: 1473. In some embodiments, the dsRNA includes (i) a sense strand having 19 contiguous nucleotides differing by no more than one, two or three nucleotides from the nucleotide sequence selected from SEQ ID NO: 1458 and (ii) an antisense strand forming a duplex with the sense strand of (i) and having 19 contiguous nucleotides differing by no more than one, two or three nucleotides from the nucleotide sequence selected from SEQ ID NO: 1473. In some embodiments, the dsRNA includes (i) a sense strand having 20 contiguous nucleotides differing by no more than one, two or three nucleotides from the nucleotide sequence selected from SEQ ID NO: 1458 and (ii) an antisense strand forming a duplex with the sense strand of (i) and having 20 contiguous nucleotides differing by no more than one, two or three nucleotides from the nucleotide sequence selected from SEQ ID NO: 1473. In some embodiments, the dsRNA includes (i) a sense strand having 21 contiguous nucleotides differing by no more than one, two or three nucleotides from the nucleotide sequence selected from SEQ ID NO: 1458 and (ii) an antisense strand forming a duplex with the sense strand of (i) and having 21 contiguous nucleotides differing by no more than one, two or three nucleotides from the nucleotide sequence selected from SEQ ID NO: 1473.

In some embodiments, the dsRNA includes (i) a sense strand having 15 contiguous nucleotides differing by no more than one, two or three nucleotides from the nucleotide sequence selected from SEQ ID NO: 1459 and (ii) an antisense strand forming a duplex with the sense strand of (i) and having 15 contiguous nucleotides differing by no more than one, two or three nucleotides from the nucleotide sequence selected from SEQ ID NO: 1474. In some embodiments, the dsRNA includes (i) a sense strand having 16 contiguous nucleotides differing by no more than one, two or three from the nucleotide sequence selected from SEQ ID NO: 1459 and (ii) an antisense strand forming a duplex with the sense strand of (i) and having 16 contiguous nucleotides differing by no more than one, two or three nucleotides from the nucleotide sequence selected from SEQ ID NO: 1474. In some embodiments, the dsRNA includes (i) a sense strand having 17 contiguous nucleotides differing by no more than one, two or three nucleotides from the nucleotide sequence selected from SEQ ID NO: 1459 and (ii) an antisense strand forming a duplex with the sense strand of (i) and having 17 contiguous nucleotides differing by no more than one, two or three nucleotides from the nucleotide sequence selected from SEQ ID NO: 1474. In some embodiments, the dsRNA includes (i) a sense strand having 18 contiguous nucleotides differing by no more than one, two or three nucleotides from the nucleotide sequence selected from SEQ ID NO: 1459 and (ii) an antisense strand forming a duplex with the sense strand of (i) and having 18 contiguous nucleotides differing by no more than one, two or three nucleotides from the nucleotide sequence selected from SEQ ID NO: 1474. In some embodiments, the dsRNA includes (i) a sense strand having 19 contiguous nucleotides differing by no more than one, two or three nucleotides from the nucleotide sequence selected from SEQ ID NO: 1459 and (ii) an antisense strand forming a duplex with the sense strand of (i) and having 19 contiguous nucleotides differing by no more than one, two or three nucleotides from the nucleotide sequence selected from SEQ ID NO: 1474. In some embodiments, the dsRNA includes (i) a sense strand having 20 contiguous nucleotides differing by no more than one, two or three nucleotides from the nucleotide sequence selected from SEQ ID NO: 1459 and (ii) an antisense strand forming a duplex with the sense strand of (i) and having 20 contiguous nucleotides differing by no more than one, two or three nucleotides from the nucleotide sequence selected from SEQ ID NO: 1474. In some embodiments, the dsRNA includes (i) a sense strand having 21 contiguous nucleotides differing by no more than one, two or three nucleotides from the nucleotide sequence selected from SEQ ID NO: 1459 and (ii) an antisense strand forming a duplex with the sense strand of (i) and having 21 contiguous nucleotides differing by no more than one, two or three nucleotides from the nucleotide sequence selected from SEQ ID NO: 1474.

In some embodiments, the dsRNA includes (i) a sense strand having 15 contiguous nucleotides differing by no more than one, two or three nucleotides from the nucleotide sequence selected from SEQ ID NO: 1460 and (ii) an antisense strand forming a duplex with the sense strand of (i) and having 15 contiguous nucleotides differing by no more than one, two or three nucleotides from the nucleotide sequence selected from SEQ ID NO: 1475. In some embodiments, the dsRNA includes (i) a sense strand having 16 contiguous nucleotides differing by no more than one, two or three from the nucleotide sequence selected from SEQ ID NO: 1460 and (ii) an antisense strand forming a duplex with the sense strand of (i) and having 16 contiguous nucleotides differing by no more than one, two or three nucleotides from the nucleotide sequence selected from SEQ ID NO: 1475. In some embodiments, the dsRNA includes (i) a sense strand having 17 contiguous nucleotides differing by no more than one, two or three nucleotides from the nucleotide sequence selected from SEQ ID NO: 1460 and (ii) an antisense strand forming a duplex with the sense strand of (i) and having 17 contiguous nucleotides differing by no more than one, two or three nucleotides from the nucleotide sequence selected from SEQ ID NO: 1475. In some embodiments, the dsRNA includes (i) a sense strand having 18 contiguous nucleotides differing by no more than one, two or three nucleotides from the nucleotide sequence selected from SEQ ID NO: 1460 and (ii) an antisense strand forming a duplex with the sense strand of (i) and having 18 contiguous nucleotides differing by no more than one, two or three nucleotides from the nucleotide sequence selected from SEQ ID NO: 1475. In some embodiments, the dsRNA includes (i) a sense strand having 19 contiguous nucleotides differing by no more than one, two or three nucleotides from the nucleotide sequence selected from SEQ ID NO: 1460 and (ii) an antisense strand forming a duplex with the sense strand of (i) and having 19 contiguous nucleotides differing by no more than one, two or three nucleotides from the nucleotide sequence selected from SEQ ID NO: 1475. In some embodiments, the dsRNA includes (i) a sense strand having 20 contiguous nucleotides differing by no more than one, two or three nucleotides from the nucleotide sequence selected from SEQ ID NO: 1460 and (ii) an antisense strand forming a duplex with the sense strand of (i) and having 20 contiguous nucleotides differing by no more than one, two or three nucleotides from the nucleotide sequence selected from SEQ ID NO: 1475. In some embodiments, the dsRNA includes (i) a sense strand having 21 contiguous nucleotides differing by no more than one, two or three nucleotides from the nucleotide sequence selected from SEQ ID NO: 1460 and (ii) an antisense strand forming a duplex with the sense strand of (i) and having 21 contiguous nucleotides differing by no more than one, two or three nucleotides from the nucleotide sequence selected from SEQ ID NO: 1475.

In some embodiments, the dsRNA includes (i) a sense strand having 15 contiguous nucleotides differing by no more than one, two or three nucleotides from the nucleotide sequence selected from SEQ ID NO: 1461 and (ii) an antisense strand forming a duplex with the sense strand of (i) and having 15 contiguous nucleotides differing by no more than one, two or three nucleotides from the nucleotide sequence selected from SEQ ID NO: 1476. In some embodiments, the dsRNA includes (i) a sense strand having 16 contiguous nucleotides differing by no more than one, two or three from the nucleotide sequence selected from SEQ ID NO: 1461 and (ii) an antisense strand forming a duplex with the sense strand of (i) and having 16 contiguous nucleotides differing by no more than one, two or three nucleotides from the nucleotide sequence selected from SEQ ID NO: 1476. In some embodiments, the dsRNA includes (i) a sense strand having 17 contiguous nucleotides differing by no more than one, two or three nucleotides from the nucleotide sequence selected from SEQ ID NO: 1461 and (ii) an antisense strand forming a duplex with the sense strand of (i) and having 17 contiguous nucleotides differing by no more than one, two or three nucleotides from the nucleotide sequence selected from SEQ ID NO: 1476. In some embodiments, the dsRNA includes (i) a sense strand having 18 contiguous nucleotides differing by no more than one, two or three nucleotides from the nucleotide sequence selected from SEQ ID NO: 1461 and (ii) an antisense strand forming a duplex with the sense strand of (i) and having 18 contiguous nucleotides differing by no more than one, two or three nucleotides from the nucleotide sequence selected from SEQ ID NO: 1476. In some embodiments, the dsRNA includes (i) a sense strand having 19 contiguous nucleotides differing by no more than one, two or three nucleotides from the nucleotide sequence selected from SEQ ID NO: 1461 and (ii) an antisense strand forming a duplex with the sense strand of (i) and having 19 contiguous nucleotides differing by no more than one, two or three nucleotides from the nucleotide sequence selected from SEQ ID NO: 1476. In some embodiments, the dsRNA includes (i) a sense strand having 20 contiguous nucleotides differing by no more than one, two or three nucleotides from the nucleotide sequence selected from SEQ ID NO: 1461 and (ii) an antisense strand forming a duplex with the sense strand of (i) and having 20 contiguous nucleotides differing by no more than one, two or three nucleotides from the nucleotide sequence selected from SEQ ID NO: 1476. In some embodiments, the dsRNA includes (i) a sense strand having 21 contiguous nucleotides differing by no more than one, two or three nucleotides from the nucleotide sequence selected from SEQ ID NO: 1461 and (ii) an antisense strand forming a duplex with the sense strand of (i) and having 21 contiguous nucleotides differing by no more than one, two or three nucleotides from the nucleotide sequence selected from SEQ ID NO: 1476.

In some embodiments, the dsRNA includes (i) a sense strand having 15 contiguous nucleotides differing by no more than one, two or three nucleotides from the nucleotide sequence selected from SEQ ID NO: 1462 and (ii) an antisense strand forming a duplex with the sense strand of (i) and having 15 contiguous nucleotides differing by no more than one, two or three nucleotides from the nucleotide sequence selected from SEQ ID NO: 1477. In some embodiments, the dsRNA includes (i) a sense strand having 16 contiguous nucleotides differing by no more than one, two or three from the nucleotide sequence selected from SEQ ID NO: 1462 and (ii) an antisense strand forming a duplex with the sense strand of (i) and having 16 contiguous nucleotides differing by no more than one, two or three nucleotides from the nucleotide sequence selected from SEQ ID NO: 1477. In some embodiments, the dsRNA includes (i) a sense strand having 17 contiguous nucleotides differing by no more than one, two or three nucleotides from the nucleotide sequence selected from SEQ ID NO: 1462 and (ii) an antisense strand forming a duplex with the sense strand of (i) and having 17 contiguous nucleotides differing by no more than one, two or three nucleotides from the nucleotide sequence selected from SEQ ID NO: 1477. In some embodiments, the dsRNA includes (i) a sense strand having 18 contiguous nucleotides differing by no more than one, two or three nucleotides from the nucleotide sequence selected from SEQ ID NO: 1462 and (ii) an antisense strand forming a duplex with the sense strand of (i) and having 18 contiguous nucleotides differing by no more than one, two or three nucleotides from the nucleotide sequence selected from SEQ ID NO: 1477. In some embodiments, the dsRNA includes (i) a sense strand having 19 contiguous nucleotides differing by no more than one, two or three nucleotides from the nucleotide sequence selected from SEQ ID NO: 1462 and (ii) an antisense strand forming a duplex with the sense strand of (i) and having 19 contiguous nucleotides differing by no more than one, two or three nucleotides from the nucleotide sequence selected from SEQ ID NO: 1477. In some embodiments, the dsRNA includes (i) a sense strand having 20 contiguous nucleotides differing by no more than one, two or three nucleotides from the nucleotide sequence selected from SEQ ID NO: 1462 and (ii) an antisense strand forming a duplex with the sense strand of (i) and having 20 contiguous nucleotides differing by no more than one, two or three nucleotides from the nucleotide sequence selected from SEQ ID NO: 1477. In some embodiments, the dsRNA includes (i) a sense strand having 21 contiguous nucleotides differing by no more than one, two or three nucleotides from the nucleotide sequence selected from SEQ ID NO: 1462 and (ii) an antisense strand forming a duplex with the sense strand of (i) and having 21 contiguous nucleotides differing by no more than one, two or three nucleotides from the nucleotide sequence selected from SEQ ID NO: 1477.

In some embodiments, the dsRNA includes (i) a sense strand having 15 contiguous nucleotides differing by no more than one, two or three nucleotides from the nucleotide sequence selected from SEQ ID NO: 1481 and (ii) an antisense strand forming a duplex with the sense strand of (i) and having 15 contiguous nucleotides differing by no more than one, two or three nucleotides from the nucleotide sequence selected from SEQ ID NO: 1299. In some embodiments, the dsRNA includes (i) a sense strand having 16 contiguous nucleotides differing by no more than one, two or three from the nucleotide sequence selected from SEQ ID NO: 1481 and (ii) an antisense strand forming a duplex with the sense strand of (i) and having 16 contiguous nucleotides differing by no more than one, two or three nucleotides from the nucleotide sequence selected from SEQ ID NO: 1299. In some embodiments, the dsRNA includes (i) a sense strand having 17 contiguous nucleotides differing by no more than one, two or three nucleotides from the nucleotide sequence selected from SEQ ID NO: 1481 and (ii) an antisense strand forming a duplex with the sense strand of (i) and having 17 contiguous nucleotides differing by no more than one, two or three nucleotides from the nucleotide sequence selected from SEQ ID NO: 1299. In some embodiments, the dsRNA includes (i) a sense strand having 18 contiguous nucleotides differing by no more than one, two or three nucleotides from the nucleotide sequence selected from SEQ ID NO: 1481 and (ii) an antisense strand forming a duplex with the sense strand of (i) and having 18 contiguous nucleotides differing by no more than one, two or three nucleotides from the nucleotide sequence selected from SEQ ID NO: 1299. In some embodiments, the dsRNA includes (i) a sense strand having 19 contiguous nucleotides differing by no more than one, two or three nucleotides from the nucleotide sequence selected from SEQ ID NO: 1481 and (ii) an antisense strand forming a duplex with the sense strand of (i) and having 19 contiguous nucleotides differing by no more than one, two or three nucleotides from the nucleotide sequence selected from SEQ ID NO: 1299. In some embodiments, the dsRNA includes (i) a sense strand having 20 contiguous nucleotides differing by no more than one, two or three nucleotides from the nucleotide sequence selected from SEQ ID NO: 1481 and (ii) an antisense strand forming a duplex with the sense strand of (i) and having 20 contiguous nucleotides differing by no more than one, two or three nucleotides from the nucleotide sequence selected from SEQ ID NO: 1299. In some embodiments, the dsRNA includes (i) a sense strand having 21 contiguous nucleotides differing by no more than one, two or three nucleotides from the nucleotide sequence selected from SEQ ID NO: 1481 and (ii) an antisense strand forming a duplex with the sense strand of (i) and having 21 contiguous nucleotides differing by no more than one, two or three nucleotides from the nucleotide sequence selected from SEQ ID NO: 1299.

In some embodiments, the dsRNA includes (i) a sense strand having 15 contiguous nucleotides differing by no more than one, two or three nucleotides from the nucleotide sequence selected from SEQ ID NO: 1482 and (ii) an antisense strand forming a duplex with the sense strand of (i) and having 15 contiguous nucleotides differing by no more than one, two or three nucleotides from the nucleotide sequence selected from SEQ ID NO: 1299. In some embodiments, the dsRNA includes (i) a sense strand having 16 contiguous nucleotides differing by no more than one, two or three from the nucleotide sequence selected from SEQ ID NO: 1482 and (ii) an antisense strand forming a duplex with the sense strand of (i) and having 16 contiguous nucleotides differing by no more than one, two or three nucleotides from the nucleotide sequence selected from SEQ ID NO: 1299. In some embodiments, the dsRNA includes (i) a sense strand having 17 contiguous nucleotides differing by no more than one, two or three nucleotides from the nucleotide sequence selected from SEQ ID NO: 1482 and (ii) an antisense strand forming a duplex with the sense strand of (i) and having 17 contiguous nucleotides differing by no more than one, two or three nucleotides from the nucleotide sequence selected from SEQ ID NO: 1299. In some embodiments, the dsRNA includes (i) a sense strand having 18 contiguous nucleotides differing by no more than one, two or three nucleotides from the nucleotide sequence selected from SEQ ID NO: 1482 and (ii) an antisense strand forming a duplex with the sense strand of (i) and having 18 contiguous nucleotides differing by no more than one, two or three nucleotides from the nucleotide sequence selected from SEQ ID NO: 1299. In some embodiments, the dsRNA includes (i) a sense strand having 19 contiguous nucleotides differing by no more than one, two or three nucleotides from the nucleotide sequence selected from SEQ ID NO: 1482 and (ii) an antisense strand forming a duplex with the sense strand of (i) and having 19 contiguous nucleotides differing by no more than one, two or three nucleotides from the nucleotide sequence selected from SEQ ID NO: 1299. In some embodiments, the dsRNA includes (i) a sense strand having 20 contiguous nucleotides differing by no more than one, two or three nucleotides from the nucleotide sequence selected from SEQ ID NO: 1482 and (ii) an antisense strand forming a duplex with the sense strand of (i) and having 20 contiguous nucleotides differing by no more than one, two or three nucleotides from the nucleotide sequence selected from SEQ ID NO: 1299. In some embodiments, the dsRNA includes (i) a sense strand having 21 contiguous nucleotides differing by no more than one, two or three nucleotides from the nucleotide sequence selected from SEQ ID NO: 1482 and (ii) an antisense strand forming a duplex with the sense strand of (i) and having 21 contiguous nucleotides differing by no more than one, two or three nucleotides from the nucleotide sequence selected from SEQ ID NO: 1299.

In some embodiments, the dsRNA includes (i) a sense strand having 15 contiguous nucleotides differing by no more than one, two or three nucleotides from the nucleotide sequence selected from SEQ ID NO: 1451 and (ii) an antisense strand forming a duplex with the sense strand of (i) and having 15 contiguous nucleotides differing by no more than one, two or three nucleotides from the nucleotide sequence selected from SEQ ID NO: 2600. In some embodiments, the dsRNA includes (i) a sense strand having 16 contiguous nucleotides differing by no more than one, two or three from the nucleotide sequence selected from SEQ ID NO: 1451 and (ii) an antisense strand forming a duplex with the sense strand of (i) and having 16 contiguous nucleotides differing by no more than one, two or three nucleotides from the nucleotide sequence selected from SEQ ID NO: 2600. In some embodiments, the dsRNA includes (i) a sense strand having 17 contiguous nucleotides differing by no more than one, two or three nucleotides from the nucleotide sequence selected from SEQ ID NO: 1451 and (ii) an antisense strand forming a duplex with the sense strand of (i) and having 17 contiguous nucleotides differing by no more than one, two or three nucleotides from the nucleotide sequence selected from SEQ ID NO: 2600. In some embodiments, the dsRNA includes (i) a sense strand having 18 contiguous nucleotides differing by no more than one, two or three nucleotides from the nucleotide sequence selected from SEQ ID NO: 1451 and (ii) an antisense strand forming a duplex with the sense strand of (i) and having 18 contiguous nucleotides differing by no more than one, two or three nucleotides from the nucleotide sequence selected from SEQ ID NO: 2600. In some embodiments, the dsRNA includes (i) a sense strand having 19 contiguous nucleotides differing by no more than one, two or three nucleotides from the nucleotide sequence selected from SEQ ID NO: 1451 and (ii) an antisense strand forming a duplex with the sense strand of (i) and having 19 contiguous nucleotides differing by no more than one, two or three nucleotides from the nucleotide sequence selected from SEQ ID NO: 2600. In some embodiments, the dsRNA includes (i) a sense strand having 20 contiguous nucleotides differing by no more than one, two or three nucleotides from the nucleotide sequence selected from SEQ ID NO: 1451 and (ii) an antisense strand forming a duplex with the sense strand of (i) and having 20 contiguous nucleotides differing by no more than one, two or three nucleotides from the nucleotide sequence selected from SEQ ID NO: 2600. In some embodiments, the dsRNA includes (i) a sense strand having 21 contiguous nucleotides differing by no more than one, two or three nucleotides from the nucleotide sequence selected from SEQ ID NO: 1451 and (ii) an antisense strand forming a duplex with the sense strand of (i) and having 21 contiguous nucleotides differing by no more than one, two or three nucleotides from the nucleotide sequence selected from SEQ ID NO: 2600.

In some embodiments, the dsRNA includes (i) a sense strand having 15 contiguous nucleotides differing by no more than one, two or three nucleotides from the nucleotide sequence selected from SEQ ID NO: 1456 and (ii) an antisense strand forming a duplex with the sense strand of (i) and having 15 contiguous nucleotides differing by no more than one, two or three nucleotides from the nucleotide sequence selected from SEQ ID NO: 2601. In some embodiments, the dsRNA includes (i) a sense strand having 16 contiguous nucleotides differing by no more than one, two or three from the nucleotide sequence selected from SEQ ID NO: 1456 and (ii) an antisense strand forming a duplex with the sense strand of (i) and having 16 contiguous nucleotides differing by no more than one, two or three nucleotides from the nucleotide sequence selected from SEQ ID NO: 2601. In some embodiments, the dsRNA includes (i) a sense strand having 17 contiguous nucleotides differing by no more than one, two or three nucleotides from the nucleotide sequence selected from SEQ ID NO: 1456 and (ii) an antisense strand forming a duplex with the sense strand of (i) and having 17 contiguous nucleotides differing by no more than one, two or three nucleotides from the nucleotide sequence selected from SEQ ID NO: 2601. In some embodiments, the dsRNA includes (i) a sense strand having 18 contiguous nucleotides differing by no more than one, two or three nucleotides from the nucleotide sequence selected from SEQ ID NO: 1456 and (ii) an antisense strand forming a duplex with the sense strand of (i) and having 18 contiguous nucleotides differing by no more than one, two or three nucleotides from the nucleotide sequence selected from SEQ ID NO: 2601. In some embodiments, the dsRNA includes (i) a sense strand having 19 contiguous nucleotides differing by no more than one, two or three nucleotides from the nucleotide sequence selected from SEQ ID NO: 1456 and (ii) an antisense strand forming a duplex with the sense strand of (i) and having 19 contiguous nucleotides differing by no more than one, two or three nucleotides from the nucleotide sequence selected from SEQ ID NO: 2601. In some embodiments, the dsRNA includes (i) a sense strand having 20 contiguous nucleotides differing by no more than one, two or three nucleotides from the nucleotide sequence selected from SEQ ID NO: 1456 and (ii) an antisense strand forming a duplex with the sense strand of (i) and having 20 contiguous nucleotides differing by no more than one, two or three nucleotides from the nucleotide sequence selected from SEQ ID NO: 2601. In some embodiments, the dsRNA includes (i) a sense strand having 21 contiguous nucleotides differing by no more than one, two or three nucleotides from the nucleotide sequence selected from SEQ ID NO: 1456 and (ii) an antisense strand forming a duplex with the sense strand of (i) and having 21 contiguous nucleotides differing by no more than one, two or three nucleotides from the nucleotide sequence selected from SEQ ID NO: 2601.

In some embodiments, the dsRNA includes (i) a sense strand having 15 contiguous nucleotides differing by no more than one, two or three nucleotides from the nucleotide sequence selected from SEQ ID NO: 1452 and (ii) an antisense strand forming a duplex with the sense strand of (i) and having 15 contiguous nucleotides differing by no more than one, two or three nucleotides from the nucleotide sequence selected from SEQ ID NO: 2602. In some embodiments, the dsRNA includes (i) a sense strand having 16 contiguous nucleotides differing by no more than one, two or three from the nucleotide sequence selected from SEQ ID NO: 1452 and (ii) an antisense strand forming a duplex with the sense strand of (i) and having 16 contiguous nucleotides differing by no more than one, two or three nucleotides from the nucleotide sequence selected from SEQ ID NO: 2602. In some embodiments, the dsRNA includes (i) a sense strand having 17 contiguous nucleotides differing by no more than one, two or three nucleotides from the nucleotide sequence selected from SEQ ID NO: 1452 and (ii) an antisense strand forming a duplex with the sense strand of (i) and having 17 contiguous nucleotides differing by no more than one, two or three nucleotides from the nucleotide sequence selected from SEQ ID NO: 2602. In some embodiments, the dsRNA includes (i) a sense strand having 18 contiguous nucleotides differing by no more than one, two or three nucleotides from the nucleotide sequence selected from SEQ ID NO: 1452 and (ii) an antisense strand forming a duplex with the sense strand of (i) and having 18 contiguous nucleotides differing by no more than one, two or three nucleotides from the nucleotide sequence selected from SEQ ID NO: 2602. In some embodiments, the dsRNA includes (i) a sense strand having 19 contiguous nucleotides differing by no more than one, two or three nucleotides from the nucleotide sequence selected from SEQ ID NO: 1452 and (ii) an antisense strand forming a duplex with the sense strand of (i) and having 19 contiguous nucleotides differing by no more than one, two or three nucleotides from the nucleotide sequence selected from SEQ ID NO: 2602. In some embodiments, the dsRNA includes (i) a sense strand having 20 contiguous nucleotides differing by no more than one, two or three nucleotides from the nucleotide sequence selected from SEQ ID NO: 1452 and (ii) an antisense strand forming a duplex with the sense strand of (i) and having 20 contiguous nucleotides differing by no more than one, two or three nucleotides from the nucleotide sequence selected from SEQ ID NO: 2602. In some embodiments, the dsRNA includes (i) a sense strand having 21 contiguous nucleotides differing by no more than one, two or three nucleotides from the nucleotide sequence selected from SEQ ID NO: 1452 and (ii) an antisense strand forming a duplex with the sense strand of (i) and having 21 contiguous nucleotides differing by no more than one, two or three nucleotides from the nucleotide sequence selected from SEQ ID NO: 2602.

In some embodiments, the dsRNA includes (i) a sense strand having 15 contiguous nucleotides differing by no more than one, two or three nucleotides from the nucleotide sequence selected from SEQ ID NO: 1454 and (ii) an antisense strand forming a duplex with the sense strand of (i) and having 15 contiguous nucleotides differing by no more than one, two or three nucleotides from the nucleotide sequence selected from SEQ ID NO: 2603. In some embodiments, the dsRNA includes (i) a sense strand having 16 contiguous nucleotides differing by no more than one, two or three from the nucleotide sequence selected from SEQ ID NO: 1454 and (ii) an antisense strand forming a duplex with the sense strand of (i) and having 16 contiguous nucleotides differing by no more than one, two or three nucleotides from the nucleotide sequence selected from SEQ ID NO: 2603. In some embodiments, the dsRNA includes (i) a sense strand having 17 contiguous nucleotides differing by no more than one, two or three nucleotides from the nucleotide sequence selected from SEQ ID NO: 1454 and (ii) an antisense strand forming a duplex with the sense strand of (i) and having 17 contiguous nucleotides differing by no more than one, two or three nucleotides from the nucleotide sequence selected from SEQ ID NO: 2603. In some embodiments, the dsRNA includes (i) a sense strand having 18 contiguous nucleotides differing by no more than one, two or three nucleotides from the nucleotide sequence selected from SEQ ID NO: 1454 and (ii) an antisense strand forming a duplex with the sense strand of (i) and having 18 contiguous nucleotides differing by no more than one, two or three nucleotides from the nucleotide sequence selected from SEQ ID NO: 2603. In some embodiments, the dsRNA includes (i) a sense strand having 19 contiguous nucleotides differing by no more than one, two or three nucleotides from the nucleotide sequence selected from SEQ ID NO: 1454 and (ii) an antisense strand forming a duplex with the sense strand of (i) and having 19 contiguous nucleotides differing by no more than one, two or three nucleotides from the nucleotide sequence selected from SEQ ID NO: 2603. In some embodiments, the dsRNA includes (i) a sense strand having 20 contiguous nucleotides differing by no more than one, two or three nucleotides from the nucleotide sequence selected from SEQ ID NO: 1454 and (ii) an antisense strand forming a duplex with the sense strand of (i) and having 20 contiguous nucleotides differing by no more than one, two or three nucleotides from the nucleotide sequence selected from SEQ ID NO: 2603. In some embodiments, the dsRNA includes (i) a sense strand having 21 contiguous nucleotides differing by no more than one, two or three nucleotides from the nucleotide sequence selected from SEQ ID NO: 1454 and (ii) an antisense strand forming a duplex with the sense strand of (i) and having 21 contiguous nucleotides differing by no more than one, two or three nucleotides from the nucleotide sequence selected from SEQ ID NO: 2603.

In some embodiments, the dsRNA includes (i) a sense strand having 15 contiguous nucleotides differing by no more than one, two or three nucleotides from the nucleotide sequence selected from SEQ ID NO: 1458 and (ii) an antisense strand forming a duplex with the sense strand of (i) and having 15 contiguous nucleotides differing by no more than one, two or three nucleotides from the nucleotide sequence selected from SEQ ID NO: 2604. In some embodiments, the dsRNA includes (i) a sense strand having 16 contiguous nucleotides differing by no more than one, two or three from the nucleotide sequence selected from SEQ ID NO: 1458 and (ii) an antisense strand forming a duplex with the sense strand of (i) and having 16 contiguous nucleotides differing by no more than one, two or three nucleotides from the nucleotide sequence selected from SEQ ID NO: 2604. In some embodiments, the dsRNA includes (i) a sense strand having 17 contiguous nucleotides differing by no more than one, two or three nucleotides from the nucleotide sequence selected from SEQ ID NO: 1458 and (ii) an antisense strand forming a duplex with the sense strand of (i) and having 17 contiguous nucleotides differing by no more than one, two or three nucleotides from the nucleotide sequence selected from SEQ ID NO: 2604. In some embodiments, the dsRNA includes (i) a sense strand having 18 contiguous nucleotides differing by no more than one, two or three nucleotides from the nucleotide sequence selected from SEQ ID NO: 1458 and (ii) an antisense strand forming a duplex with the sense strand of (i) and having 18 contiguous nucleotides differing by no more than one, two or three nucleotides from the nucleotide sequence selected from SEQ ID NO: 2604. In some embodiments, the dsRNA includes (i) a sense strand having 19 contiguous nucleotides differing by no more than one, two or three nucleotides from the nucleotide sequence selected from SEQ ID NO: 1458 and (ii) an antisense strand forming a duplex with the sense strand of (i) and having 19 contiguous nucleotides differing by no more than one, two or three nucleotides from the nucleotide sequence selected from SEQ ID NO: 2604. In some embodiments, the dsRNA includes (i) a sense strand having 20 contiguous nucleotides differing by no more than one, two or three nucleotides from the nucleotide sequence selected from SEQ ID NO: 1458 and (ii) an antisense strand forming a duplex with the sense strand of (i) and having 20 contiguous nucleotides differing by no more than one, two or three nucleotides from the nucleotide sequence selected from SEQ ID NO: 2604. In some embodiments, the dsRNA includes (i) a sense strand having 21 contiguous nucleotides differing by no more than one, two or three nucleotides from the nucleotide sequence selected from SEQ ID NO: 1458 and (ii) an antisense strand forming a duplex with the sense strand of (i) and having 21 contiguous nucleotides differing by no more than one, two or three nucleotides from the nucleotide sequence selected from SEQ ID NO: 2604.

In some embodiments, the dsRNA includes (i) a sense strand having 15 contiguous nucleotides differing by no more than one, two or three nucleotides from the nucleotide sequence selected from SEQ ID NO: 1460 and (ii) an antisense strand forming a duplex with the sense strand of (i) and having 15 contiguous nucleotides differing by no more than one, two or three nucleotides from the nucleotide sequence selected from SEQ ID NO: 2605. In some embodiments, the dsRNA includes (i) a sense strand having 16 contiguous nucleotides differing by no more than one, two or three from the nucleotide sequence selected from SEQ ID NO: 1460 and (ii) an antisense strand forming a duplex with the sense strand of (i) and having 16 contiguous nucleotides differing by no more than one, two or three nucleotides from the nucleotide sequence selected from SEQ ID NO: 2605. In some embodiments, the dsRNA includes (i) a sense strand having 17 contiguous nucleotides differing by no more than one, two or three nucleotides from the nucleotide sequence selected from SEQ ID NO: 1460 and (ii) an antisense strand forming a duplex with the sense strand of (i) and having 17 contiguous nucleotides differing by no more than one, two or three nucleotides from the nucleotide sequence selected from SEQ ID NO: 2605. In some embodiments, the dsRNA includes (i) a sense strand having 18 contiguous nucleotides differing by no more than one, two or three nucleotides from the nucleotide sequence selected from SEQ ID NO: 1460 and (ii) an antisense strand forming a duplex with the sense strand of (i) and having 18 contiguous nucleotides differing by no more than one, two or three nucleotides from the nucleotide sequence selected from SEQ ID NO: 2605. In some embodiments, the dsRNA includes (i) a sense strand having 19 contiguous nucleotides differing by no more than one, two or three nucleotides from the nucleotide sequence selected from SEQ ID NO: 1460 and (ii) an antisense strand forming a duplex with the sense strand of (i) and having 19 contiguous nucleotides differing by no more than one, two or three nucleotides from the nucleotide sequence selected from SEQ ID NO: 2605. In some embodiments, the dsRNA includes (i) a sense strand having 20 contiguous nucleotides differing by no more than one, two or three nucleotides from the nucleotide sequence selected from SEQ ID NO: 1460 and (ii) an antisense strand forming a duplex with the sense strand of (i) and having 20 contiguous nucleotides differing by no more than one, two or three nucleotides from the nucleotide sequence selected from SEQ ID NO: 2605. In some embodiments, the dsRNA includes (i) a sense strand having 21 contiguous nucleotides differing by no more than one, two or three nucleotides from the nucleotide sequence selected from SEQ ID NO: 1460 and (ii) an antisense strand forming a duplex with the sense strand of (i) and having 21 contiguous nucleotides differing by no more than one, two or three nucleotides from the nucleotide sequence selected from SEQ ID NO: 2605.

In certain aspects, when a sense strand or an antisense strand of a dsRNA in above paragraphs is differing by a certain number of nucleotides (e.g., one, two or three nucleotides) from a specific sequence (e.g., SEQ ID NOs: 1294 to 1301, 1448 to 1477, 1481 to 1482, and 2600 to 2605), it is meant by that the sense strand or the antisense strand includes one, two or three nucleotides, having different nucleobases and/or different modifications compared to the nucleobases and/or the modifications of the nucleotides at the corresponding positions of the specific sequence (e.g., SEQ ID NOs: 1294 to 1301, 1448 to 1477, 1481 to 1482, and 2600 to 2605). In some embodiments, when a sense strand or an antisense strand is differing by a certain number of nucleotides (e.g., one, two or three nucleotides) from a specific sequence (e.g., SEQ ID NOs: 1294 to 1301, 1448 to 1477, 1481 to 1482, and 2600 to 2605), the sense strand or the antisense strand includes one, two, or three nucleotides, having different nucleobases compared to the nucleobases of the nucleotides at the corresponding positions of the specific sequence (e.g., SEQ ID NOs: 1294 to 1301, 1448 to 1477, 1481 to 1482, and 2600 to 2605). In some embodiments, when a sense strand or an antisense strand is differing by a certain number of nucleotides (e.g., one, two or three nucleotides) from a specific sequence (e.g., SEQ ID NOs: 1294 to 1301, 1448 to 1477, 1481 to 1482, and 2600 to 2605), the sense strand or the antisense strand includes one, two, or three nucleotides, having different modifications compared to the modifications of the nucleotides at the corresponding positions of the specific sequence (e.g., SEQ ID NOs: 1294 to 1301, 1448 to 1477, 1481 to 1482, and 2600 to 2605). In some embodiments, when a sense strand or an antisense strand is differing by a certain number of nucleotides (e.g., one, two or three nucleotides) from a specific sequence (e.g., SEQ ID NOs: 1294 to 1301, 1448 to 1477, 1481 to 1482, and 2600 to 2605), the sense strand or the antisense strand includes one, two, or three nucleotides having different nucleobases and different modifications compared to the nucleobases and the modifications of the nucleotides at the corresponding positions of the specific sequence (e.g., SEQ ID NOs: 1294 to 1301, 1448 to 1477, 1481 to 1482, and 2600 to 2605).

In certain aspects, when a sense strand or an antisense strand of a dsRNA in above paragraphs is differing by a certain number of nucleotides (e.g., one, two or three nucleotides) from a specific sequence (e.g., SEQ ID NOs: 1294 to 1301, 1448 to 1477, 1481 to 1482, and 2600 to 2605), it is meant by that the sense strand or the antisense strand includes one, two or three nucleotides, having different nucleobases, different modifications, and/or different phosphate linkages (e.g., phosphorothioate (PS)), compared to the nucleobases, the modifications, and/or the phosphate linkages of the nucleotides at the corresponding positions of the specific sequence (e.g., SEQ ID NOs: 1294 to 1301, 1448 to 1477, 1481 to 1482, and 2600 to 2605). In some embodiments, when a sense strand or an antisense strand is differing by a certain number of nucleotides (e.g., one, two or three nucleotides) from a specific sequence (e.g., SEQ ID NOs: 1294 to 1301, 1448 to 1477, 1481 to 1482, and 2600 to 2605), the sense strand or the antisense strand includes one, two, or three nucleotides, having different phosphate linkages compared to the phosphate linkages of the nucleotides at the corresponding positions of the specific sequence (e.g., SEQ ID NOs: 1294 to 1301, 1448 to 1477, 1481 to 1482, and 2600 to 2605). In some embodiments, when a sense strand or an antisense strand is differing by a certain number of nucleotides (e.g., one, two or three nucleotides) from a specific sequence (e.g., SEQ ID NOs: 1294 to 1301, 1448 to 1477, 1481 to 1482, and 2600 to 2605), the sense strand or the antisense strand includes one, two, or three nucleotides, having different nucleobases and different phosphate linkages compared to the nucleobases and the phosphate linkages of the nucleotides at the corresponding positions of the specific sequence (e.g., SEQ ID NOs: 1294 to 1301, 1448 to 1477, 1481 to 1482, and 2600 to 2605). In some embodiments, when a sense strand or an antisense strand is differing by a certain number of nucleotides (e.g., one, two or three nucleotides) from a specific sequence (e.g., SEQ ID NOs: 1294 to 1301, 1448 to 1477, 1481 to 1482, and 2600 to 2605), the sense strand or the antisense strand includes one, two, or three nucleotides, having different modifications and different phosphate linkages compared to the modifications and the phosphate linkages of the nucleotides at the corresponding positions of the specific sequence (e.g., SEQ ID NOs: 1298 to 1301, 1463 to 1477, and 2600 to 2605). In some embodiments, when a sense strand or an antisense strand is differing by a certain number of nucleotides (e.g., one, two or three nucleotides) from a specific sequence (e.g., SEQ ID NOs: 1294 to 1301, 1448 to 1477, 1481 to 1482, and 2600 to 2605), the sense strand or the antisense strand includes one, two, or three nucleotides having different nucleobases, different modifications, and different phosphate linkages compared to the nucleobases, the modifications, and the phosphate linkages of the nucleotides at the corresponding positions of the specific sequence (e.g., SEQ ID NOs: 1294 to 1301, 1448 to 1477, 1481 to 1482, and 2600 to 2605).

### Ligands

In an aspect, a ligand including the various chemical and biological moieties, such as a small molecule compound, a peptide, an antibody, a carbohydrate, or an additional nucleic acid with or without a linker, can be coupled or conjugated to a dsRNA as described herein. In certain aspects, the ligand may directly (e.g., covalently) conjugated to at least one strand of the dsRNA.

In certain aspects, the ligand may be conjugated via a linker thereof (e.g., covalent linker) to a sense strand. In some embodiments, the ligand may be conjugated via a linker (e.g., covalent linker, or by forming a phosphate or phosphorothioate linkage) to one or more nucleotides in the sense strand. In some embodiments, the ligand may be conjugated via a linker (e.g., covalent linker, or by forming a phosphate or phosphorothioate linkage) to 5′ end of the sense strand. In some embodiments, the ligand may be conjugated via a linker (e.g., covalent linker, or by forming a phosphate or phosphorothioate linkage) to 3′ end of the sense strand. In some embodiments, the ligand may be conjugated via a linker (e.g., covalent linker, or by forming a phosphate or phosphorothioate linkage) to 5′ carbon of the first nucleotide from the 5′ end. In some embodiments, the ligand may be conjugated via a linker (e.g., covalent linker, or by forming a phosphate or phosphorothioate linkage) to 3′ carbon of the first nucleotide from the 3′ end.

In certain aspects, the ligand may be conjugated via a linker (e.g., covalent linker) to an antisense strand. In some embodiments, the ligand may be conjugated via a linker (e.g., covalent linker, or by forming phosphate or phosphorothioate linkage) to one or more nucleotides of an antisense strand. In some embodiments, the ligand may be conjugated via a linker (e.g., covalent linker, or by forming phosphate or phosphorothioate linkage) to 5′ end of an antisense strand. In some embodiments, the ligand may be conjugated via a linker (e.g., covalent linker, or by forming phosphate or phosphorothioate linkage) to 3′ end of an antisense strand. In some embodiments, the ligand may be conjugated via a linker (e.g., covalent linker, or by forming phosphate or phosphorothioate linkage) to 5′ carbon of the first nucleotide from the 5′ end. In some embodiments, the ligand may be conjugated via a linker (e.g., covalent linker, or by forming phosphate or phosphorothioate linkage) to 3′ carbon of the first nucleotide from the 3′ end.

In some embodiments, when the linker forms a phosphate or phosphorothioate linkage, one or more oxygens in the phosphate or phosphorothioate group may be provided from the conjugating nucleotide and/or the ligand. In some embodiments, the linker forms a phosphate or phosphorothioate linkage including the oxygen atom from hydroxyl group of the first nucleotide (e.g., 5′-OH at 5′ end, or 3′-OH from the 3′-end). In some embodiments, the linker forms a phosphate or phosphorothioate linkage including the oxygen atom from the ligand (e.g., terminal group containing oxygen). In some embodiments, the linker forms a phosphate or phosphorothioate linkage including the oxygen atom from hydroxyl group of the first nucleotide (e.g., 5′-OH at 5′ end, or 3′-OH from the 3′-end) and the oxygen atom from the ligand (e.g., terminal group containing oxygen).

In certain aspects, the linker may be a cleavable chemical moiety which is sufficiently stable outside the cell but which upon is spontaneously and/or irreversibly cleaved to release one or more conjugated groups (e.g., targeting moiety) when introduced in a cell or other physiological conditions (e.g., serum, or blood). In some embodiments, the cleavable linker may include a cleavage site at its terminal part that is attached to other compounds or molecules. In some embodiments, the cleavable linker may include a cleavage site that locates between the two-terminus attached to each different compound or molecule.

In certain aspects, the linker may be a non-cleavable linker. In certain aspects, the linker may be a hydrolysable linker.

A choice of the ligand may provide an enhanced affinity and/or delivery of the dsRNA to a specific target biomolecule, cell, tissue, organ compartment, or organ or region of a body. In certain aspects, the ligand may include a targeting moiety or group which bind to a specific organ cell, e.g., liver or kidney cell. In some embodiments, the ligand may include a targeting moiety or group which bind to a specific cell type, e.g., a cancer cell, endothelial cell, or bone cell. In certain aspects, the ligand may include a targeting moiety to hormones and hormone receptors. In certain aspects, the ligand may include a targeting moiety including a lipid component (e.g., short/long chain fatty acid, cationic lipid, lipophilic molecule, cholesterol, steroid, uvaol, hecigenin, diosgenin, terpene, triterpene, sarsasapogenin, friedelin, epifriedelanol-derivatized lithocholic acid, etc.) to modulate or control the binding, to increase resistance to degradation, or to increase targeting or transport into a target cell membrane or cellular lipid vesicles.

Non limiting examples of ligands may include, but not be limited to, proteins (e.g., thyrotropin, melanotropin, lectin, glycoprotein such as transferrin, or surfactant protein A), carbohydrates (e.g., mucin carbohydrate, multivalent lactose, multivalent galactose, N-acetyl-galactosamine, N-acetyl-glucosamine, multivalent mannose, multivalent fucose, glycosylated polyaminoacids, or multivalent galactose), small molecule drugs (e.g., bisphosphonate), polymers (e.g., PEG (e.g., PEG-40K), MPEG, [MPEG]2, polyglutamate, or polyaspartate), a lipid component (e.g., cholesterol, a steroid, bile acid, cholic acid, adamantane acetic acid, 1-pyrene butyric acid, dihydrotestosterone, 1,3-bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, or 03-(oleoyl)cholenic acid), organic compounds (e.g., dimethoxytrityl, or phenoxazine), vitamins (e.g., folate, vitamin B12, or biotin), small peptides (e.g., antennapedia peptide, TAT peptide, RGD peptide, an RGD peptide mimetic), an additional nucleic acids (e.g., an aptamer), dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases or a chelator (e.g., EDTA), radiolabeled markers, enzymes, or the like.

In some embodiments, the ligand may include carbohydrates (e.g., mucin carbohydrate, multivalent lactose, multivalent galactose, N-acetyl-galactosamine, N-acetyl-glucosamine multivalent mannose, multivalent fucose, glycosylated polyaminoacids, or multivalent galactose) as the targeting moiety. In some embodiments, the ligand may include multivalent lactose or multivalent galactose. In some embodiments, the ligand may include N-acetyl-galactosamine as the targeting moiety.

In certain aspects, the ligand may include one or more diagnostic compound, reporter group, cross-linking agent, nuclease-resistance conferring moiety, modified or unmodified nucleobase, lipophilic molecule, cholesterol, lipid, lectin, steroid, uvaol, hecigenin, diosgenin, terpene, triterpene, sarsasapogenin, friedelin, epifriedelanol-derivatized lithocholic acid, vitamin, carbohydrate, dextran, pullulan, chitin, chitosan, synthetic carbohydrate, oligo lactate (e.g., 15-mer), natural polymer, low- or medium-molecular weight polymer, inulin, cyclodextrin, hyaluronic acid, protein, protein-binding agent, integrin-targeting molecule, polycationic, peptide, polyamine, peptide mimic, and/or transferrin.

In certain aspects, the ligand targets a specific receptor on a cell (e.g., liver cell or kidney cell). In some embodiments, the ligand targets a cell surface protein, e.g., asialoglycoprotein receptor (ASGPR), which is abundantly expressed on liver cells (hepatocytes). In some embodiments, for targeting ASGPR, the ligand may include one or more selected from carbohydrate (e.g., pyranose such as glucose or its derivatives (e.g., GluNAc), galactose or its derivatives (e.g., GalNAc), mannose or its derivatives (e.g., mannose-6P)). In some embodiments, the ligand may include a sugar cluster containing two or more sugar moieties (e.g., glucose or its derivatives, galactose or its derivatives (e.g., GalNAc), mannose or its derivatives (e.g., mannose-6P), and etc.). In some embodiments, the ligand may include galactose cluster, e.g., GalNAc cluster, or mannose cluster. In some embodiments, the cluster may be formed by linking or coupling the sugar moieties via one or more covalent linkers.

In certain aspects, the ligand includes one or more GalNAc moieties. In some embodiments, the ligand includes one GalNAc moiety. In some embodiments, the ligand includes two GalNAc moieties. In some embodiments, the ligand includes three GalNAc moieties. In some embodiments, the ligand may include one or more covalent linkers.

In certain aspects, the ligand has a structure of Formula (A):

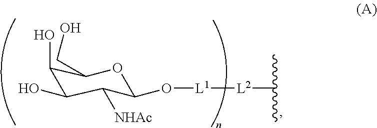

or a pharmaceutically acceptable salt thereof, wherein:
each L¹ is an independently a linker which may be same or different in each occurrence; L² is a linker;
n is an integer from 1 to 3; and
[symbol] is an attachment point to the sense strand or the antisense strand, or to a conjugate linker conjugated to the sense strand or the antisense strand.

In some embodiments, the attachment point is connected to the sense strand or the antisense strand via a direct bond. In some embodiments, the attachment point is connected to the sense strand or the antisense strand via “a conjugate linker” that connects the ligand and one or both strands of dsRNA (e.g., sense strand or antisense strand). In some embodiments, the attachment point is connected to the sense strand or the antisense strand via the conjugate linker that may form a phosphodiester linkage. In some embodiments, the attachment point is connected to the sense strand or the antisense strand via a conjugate linker that may form a phosphorothioate linkage.

In some embodiments, the attachment point is connected to the sense strand via a phosphodiester linkage at the 3′ end of the sense strand. In some embodiments, the attachment point is connected to the sense strand via a phosphodiester linkage at the 5′ end of the sense strand. In some embodiments, the attachment point is connected to the sense strand via a phosphorothioate group at the 3′ end of the sense strand. In some embodiments, the attachment point is connected to the sense strand via a phosphorothioate group at the 5′ end of the sense strand.

In some embodiments, the attachment point is connected to the antisense strand via a phosphodiester linkage at the 3′ end of the antisense strand. In some embodiments, the attachment point is connected to the antisense strand via a phosphodiester linkage at the 5′ end of the antisense strand. In some embodiments, the attachment point is connected to the antisense strand via a phosphorothioate group at the 3′ end of the antisense strand. In some embodiments, the attachment point is connected to the antisense strand via a phosphorothioate group at the 5′ end of the antisense strand.

In certain aspects, each Lⁱ is independently a covalent linker having the formula -L^{1A}-L^{1B}-L^{1C}-L^{1D}-L^{1E}-. Each L^{1A}, L^{1B}, L^{1C}, L^{1D}, and L^{1E} is independently a bond, —O—P(S)(O)—O—, or —O—P(O)(O)—O—, —O—P(S)(O)—, or —O—P(O)(O)—, a substituted or unsubstituted alkylene (e.g., C₁-C₃₀ alkylene, C₁-C₂₅ alkylene, C₁-C₁₂ or C₁-C₈ alkylene), a substituted or unsubstituted heteroalkylene (e.g., 2 to 30 membered heteroalkylene, 2 to 15 membered heteroalkylene, 2 to 12 membered heteroalkylene, or 2 to 8 membered heteroalkylene), a substituted or unsubstituted cycloalkylene (e.g., C₄-C₁₂ cycloalkylene), a substituted or unsubstituted heterocycloalkylene (e.g., 2 to 30 membered, 2 to 15 membered, or 2 to 12 membered heteroalkylene heterocycloalkylene), substituted or unsubstituted arylene (e.g., C₆-C₁₂ arylene), or a substituted or unsubstituted heteroarylene (e.g., 5 to 6 membered heteroarylene). In some embodiments, each L^{1A}, L^{1B}, L^{1C}, L^{1D}, and L^{1E} is independently a bond, a substituted or unsubstituted alkylene (e.g., C₁-C₃₀, C₁-C₁₅, or C₁-C₁₂ alkylene), a substituted or unsubstituted heteroalkylene (e.g., 2 to 12 membered heteroalkylene), substituted or unsubstituted arylene (e.g., phenylene), or substituted or unsubstituted heteroarylene (e.g., pyridylene). In some embodiments, each L^{1A}L^{1B}, L^{1C}, L^{1D}, and L^{1E} is independently a bond, unsubstituted C₁-C₁₂ alkylene, —NHC(O)—, —C(O)NH—, —(CH₂)ₐ₁—O—, —O—(CH₂)ₐ₁—, —(CH₂CH₂O)_{b1}—, —(OCH₂CH₂)_{b1}—, —O—P(S)(O)—O—, or —O—P(O)(O)—O—, and each a1 or b1 is independently an integer from 0 to 12.

In some embodiments, Li has the following structure:

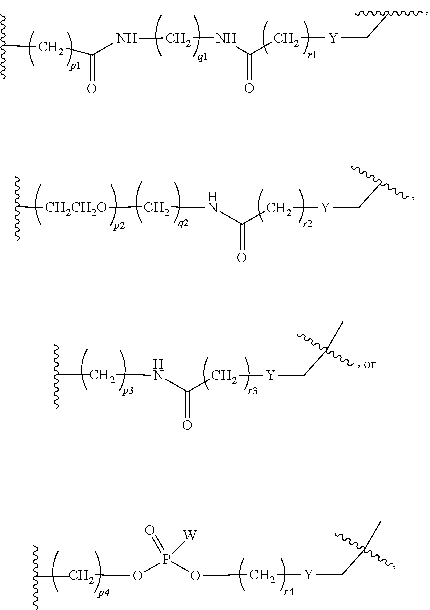

or a pharmaceutically acceptable salt thereof,
wherein:
   each p1, p2, p3, p4, q1, q2, r1, r2, r3 and r4 is independently an integer from 0 to 12;
   W is —OH or —SH; and
   Y is —O— or absent.

In some embodiments, W is —OH. In some embodiments, W is —SH.

In some embodiments, Y is —O—. In some embodiments, Y is absent.

In some embodiments, L¹ has the following structure:

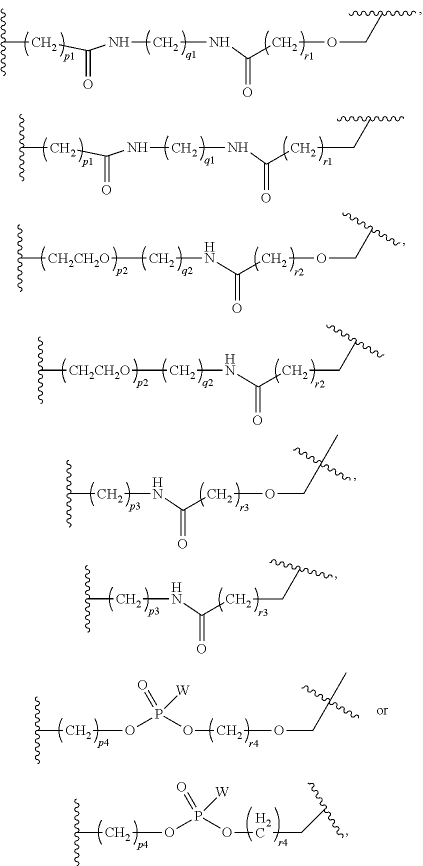

or a pharmaceutically acceptable salt thereof.
p1, p2, p3, p4, q1, q2, r1, r2, r3, r4, and W are as described above.

In some embodiments, each p1, p2, p3, p4, q1, q2, r1, r2, r3 and r4 is independently an integer from 1 to 6. In some embodiments, each p1, p2, p3, and p4 is independently 2, 3, 4, 5, 6, or 8. In some embodiments, each q1 and q2 is independently 1, 2, 3, or 4. In some embodiments, each r1, r2, r3 and r4 is independently 1, 2, 3, or 4.

In certain aspects, the ligand includes the following structures (e.g., GalNAc moiety):

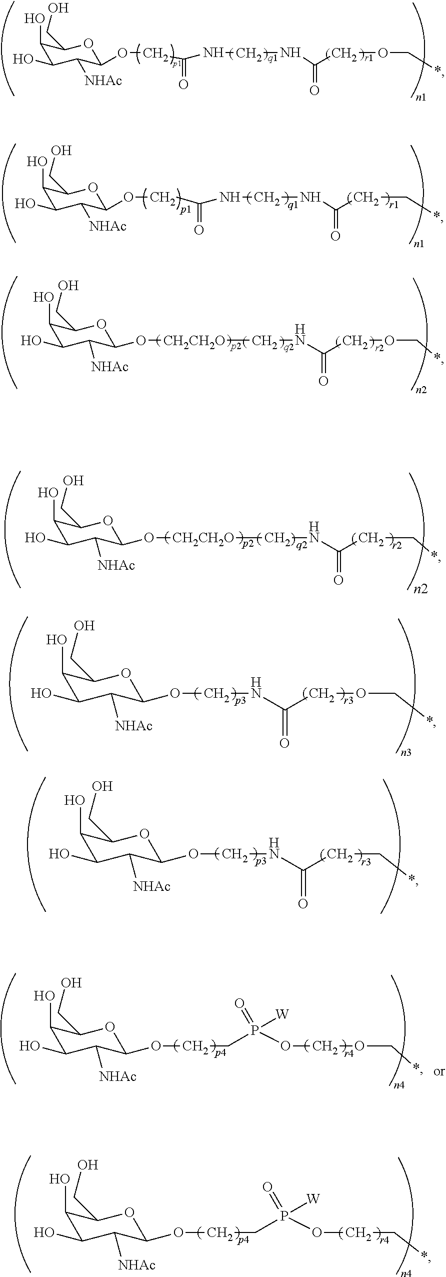

or a pharmaceutically acceptable salt thereof,
wherein each n1, n2, n3, and n4 is independently an integer from 1 to 3.
Y, W, p1, p2, p3, p4, q1, q2, r1, r2, r3, and r4 are as described above, and “*” is an attachment point to L² in Formula (A).

In some embodiments, n1 is 1. In some embodiments, n1 is 2. In some embodiments, n1 is 3. In some embodiments, n2 is 1. In some embodiments, n2 is 2. In some embodiments, n2 is 3. In some embodiments, n3 is 1. In some embodiments, n3 is 2. In some embodiments, n3 is 3. In some embodiments, n4 is 1. In some embodiments, n4 is 2. In some embodiments, n4 is 3.

In some embodiments, the ligand includes the following structure:

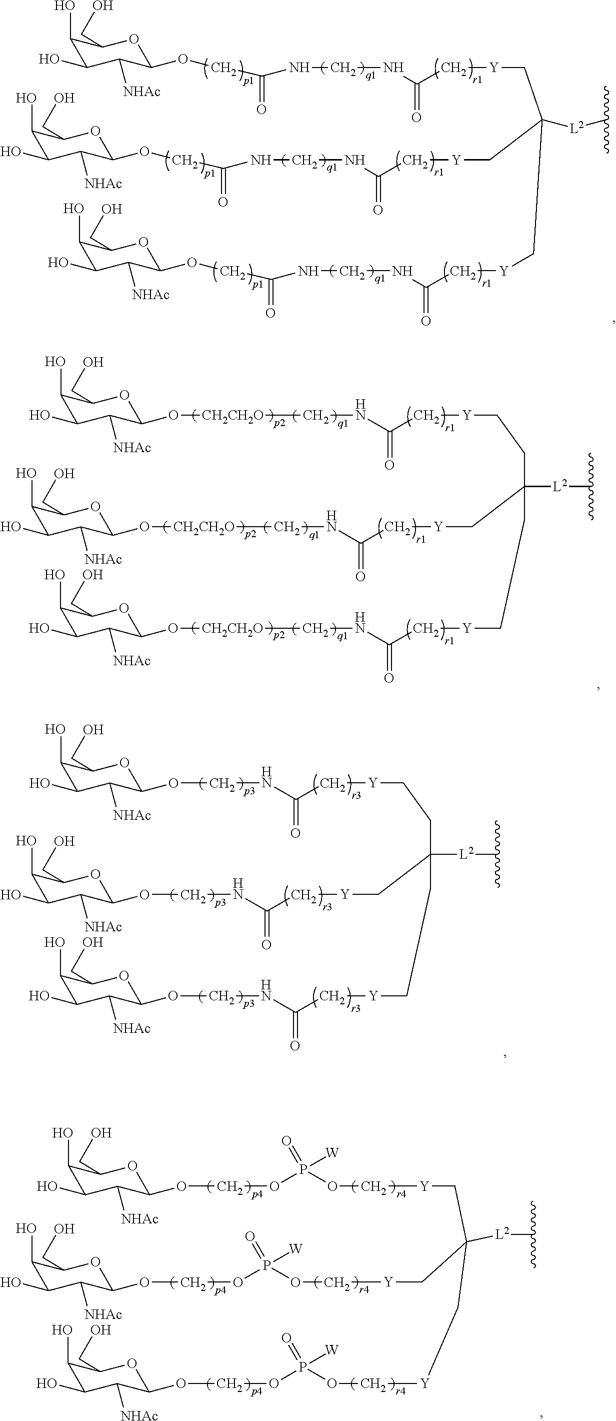

or a pharmaceutically acceptable salt thereof,
wherein L², Y, W, p1, p2, p3, p4, q1, q2, r1, r2, r3, and r4 are as described above and [symbol] is an attachment point to the sense strand or the antisense strand, or to a conjugate linker conjugated to the sense strand or the antisense strand.

In certain aspects, L² is a covalent linker of the formula -L^{2A}-L^{2B}-L^{2C}-L^{2D}-L^{2E}-. Each L^{2A}, L^{2B}, L^{2C}, L^{2D}, and L^{2E} a bond, —O—P(S)(O)—O—, —O—P(O)(O)—O—, —O—P(S)(O)—, or —O—P(O)(O)—, a substituted or unsubstituted alkylene (e.g., C₁-C₃₀ alkylene, C₁-C₂₅ alkylene, C₁-C₁₂ or C₁-C₈ alkylene), a substituted or unsubstituted heteroalkylene (e.g., 2 to 30 membered heteroalkylene, 2 to 15 membered heteroalkylene, 2 to 12 membered heteroalkylene, or 2 to 8 membered heteroalkylene), a substituted or unsubstituted cycloalkylene (e.g., C₄-C₁₂ cycloalkylene), a substituted or unsubstituted heterocycloalkylene (e.g., 5 to 6 membered heterocycloalkylene), substituted or unsubstituted arylene (e.g., phenylene), or a substituted or unsubstituted heteroarylene (e.g., 5 to 6 membered heteroarylene). In some embodiments, each L^{2A}, L^{2B} L²c L^{2D}, and L^{2E} is independently a bond, substituted or unsubstituted alkylene (e.g., C₁-C₃₀ alkylene, C₁-C₂₅ alkylene, C₁-C₁₂ or C₁-C₅ alkylene), a substituted or unsubstituted heteroalkylene (e.g., 2 to 30 membered, 2 to 15 membered, or 2 to 12 membered heteroalkylene heteroalkylene), or a substituted or unsubstituted heterocycloalkylene (e.g., 5 to 6 membered heterocycloalkylene). In some embodiments, each L^{2A}, L^{2B}, L^{2C}, L^{2D}, and L^{2E} is independently a bond, substituted (e.g., OH-substituted) or unsubstituted C₁-C₁₂ alkylene, —NHC(O)—, —C(O)NH—, —(CH₂)ₐ₂—O—, —O—(CH₂)ₐ₂—, —(CH₂CH₂O)_{b2}—, —(OCH₂CH₂)_{b2}—, —O—P(S)(O)—O—, —O—P(O)(O)—O—, —O—P(S)(O)—, or —O—P(O)(O)—, substituted or unsubstituted cycloalkylene (e.g., cyclohexylene), substituted or unsubstituted heterocycloalkylene (e.g., pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin) or substituted or unsubstituted arylene (e.g., phenylene). Each a2 or b2 is independently an integer from 1 to 12.

In some embodiments, L^{2A} is —NHC(O)—, or —C(O)NH—. In some embodiments, L^{2A} is —NHC(O)—.

In some embodiments, L^{2D} is

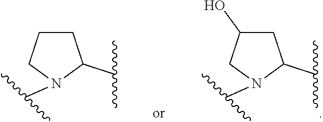 [SMILES: *C1CCCN1* ; *C1CC(O)CN1*]

In some embodiments, L^{2D} is

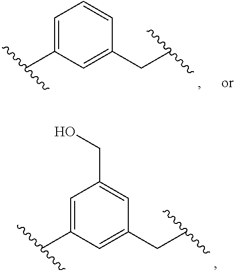 [SMILES: *Cc1cccc(*)c1 ; *Cc1cc(*)cc(CO)c1]

In some embodiments, L^{2D} is

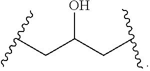 [SMILES: *CC(O)C*]

In some embodiments, L^{2D} is

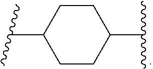 [SMILES: *C1CCC(*)CC1]

In some embodiments, L^{2D} is

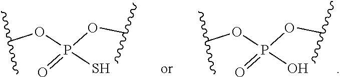 [SMILES: *OP(=O)(S)O* ; *OP(=O)(O)O*]

In some embodiments, L^{2D} is —O— or —S—.

In some embodiments, L^{2E} is a bond. In some embodiments, L^{2E} is —O— or —S—. In some embodiments, L^{2E} is

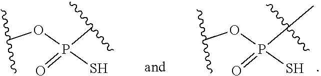 [SMILES: *OP(*)(=O)S ; *OP(*)(=O)S]

In some embodiments, L^{2E} is

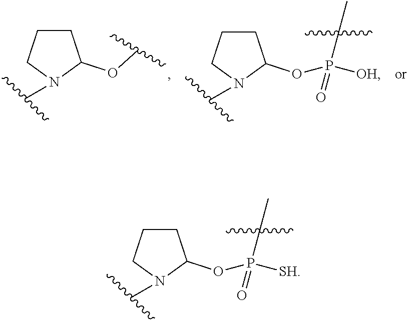 [SMILES: *OC1CCCN1* ; *N1CCCC1OP(*)(=O)O ; *N1CCCC1OP(*)(=O)S]

In some embodiments, L^{2E} is

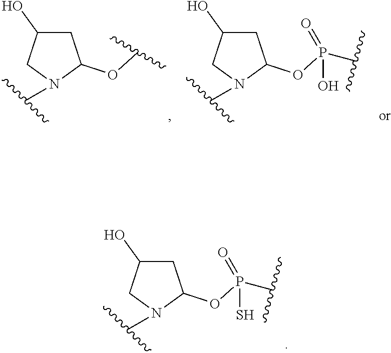 [SMILES: *OC1CC(O)CN1* ; *N1CC(O)CC1OP(*)(=O)O ; *N1CC(O)CC1OP(*)(=O)S]

In some embodiments, L^{2E} is

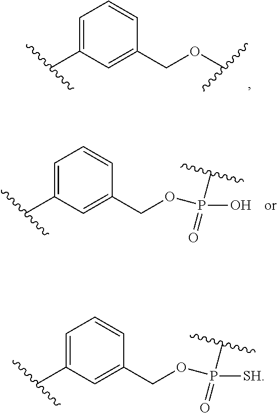 [SMILES: *OCc1cccc(*)c1 ; *c1cccc(COP(*)(=O)O)c1 ; *c1cccc(COP(*)(=O)S)c1]

In some embodiments, L^{2E} is

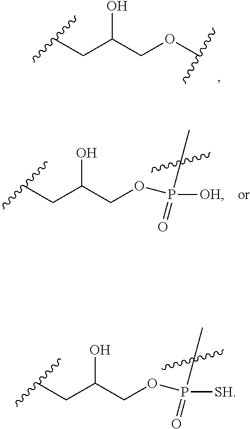 [SMILES: *CC(O)CO* ; *CC(O)COP(*)(=O)O ; *CC(O)COP(*)(=O)S]

In some embodiments,

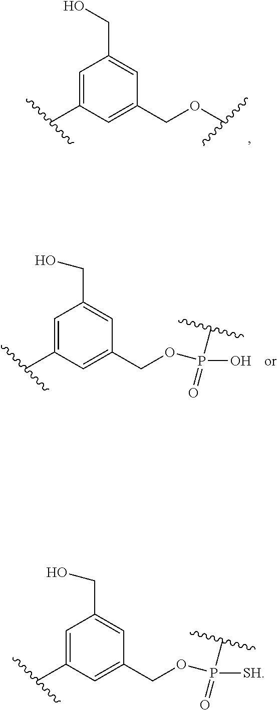 [SMILES: *OCc1cc(*)cc(CO)c1 ; *c1cc(CO)cc(COP(*)(=O)O)c1 ; *c1cc(CO)cc(COP(*)(=O)S)c1]

In some embodiments, L^{2E} is

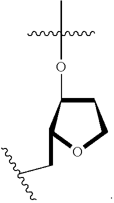

In some embodiments, the ligand includes the following structures (B-1) to (B-6):

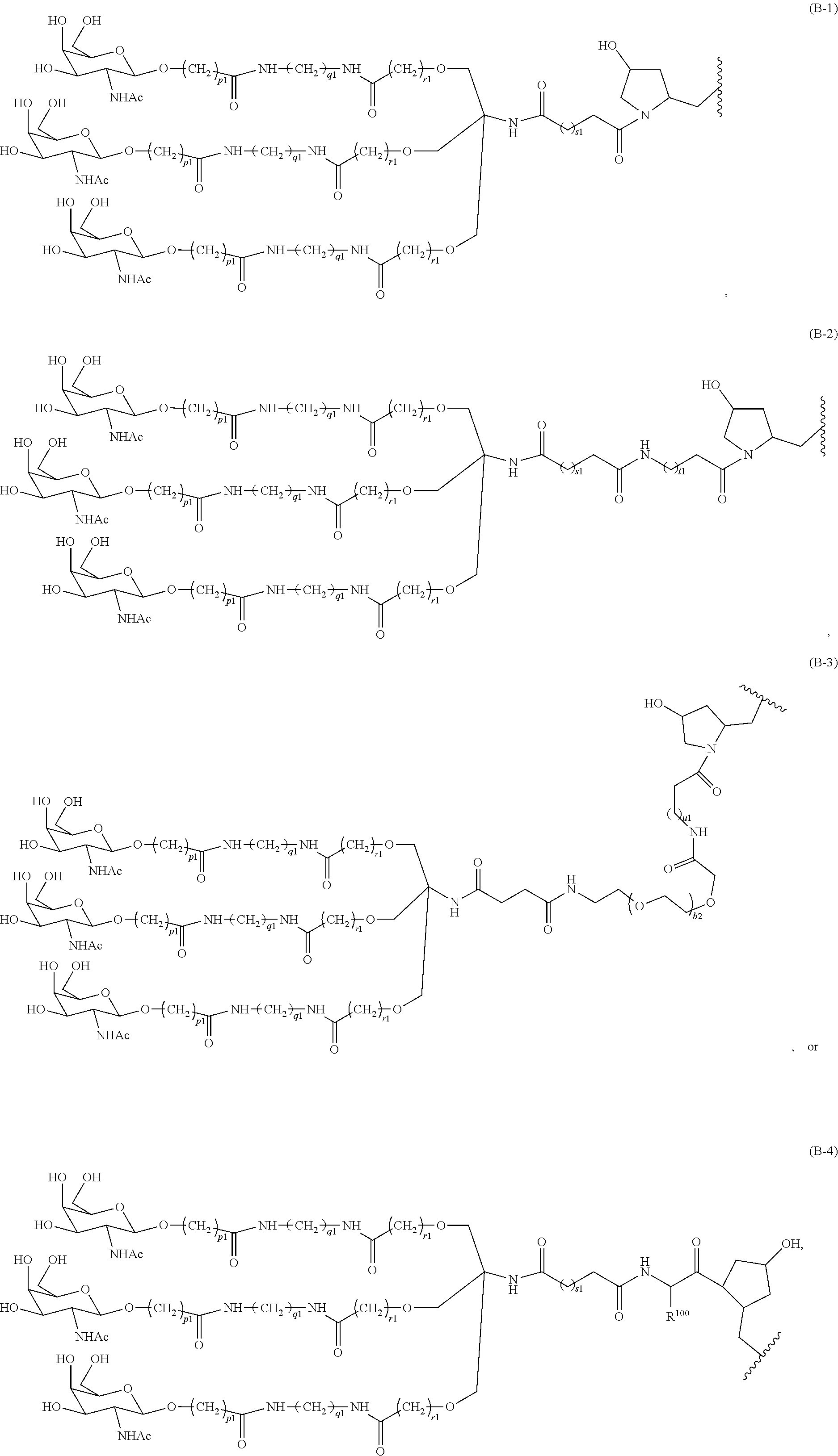

or a pharmaceutically acceptable salt thereof,
wherein each s1, t1, and u1 is independently an integer from 1 to 12, and R¹⁰⁰ is a hydrogen or a substituent (e.g., a side chain of a natural or unnatural amino acid). p1, q1, r1, and b2 are as described above.

Additional suitable ligands related to the above structures of Formula (B) and its subordinates and synthesis thereof are also described in WO2009/082607, entire contents of which are incorporated herein by reference.

In some embodiments, Y is —O—. In some embodiments, Y is absent.

In some embodiments, the ligand includes the following structures (C-1) to (C-3):

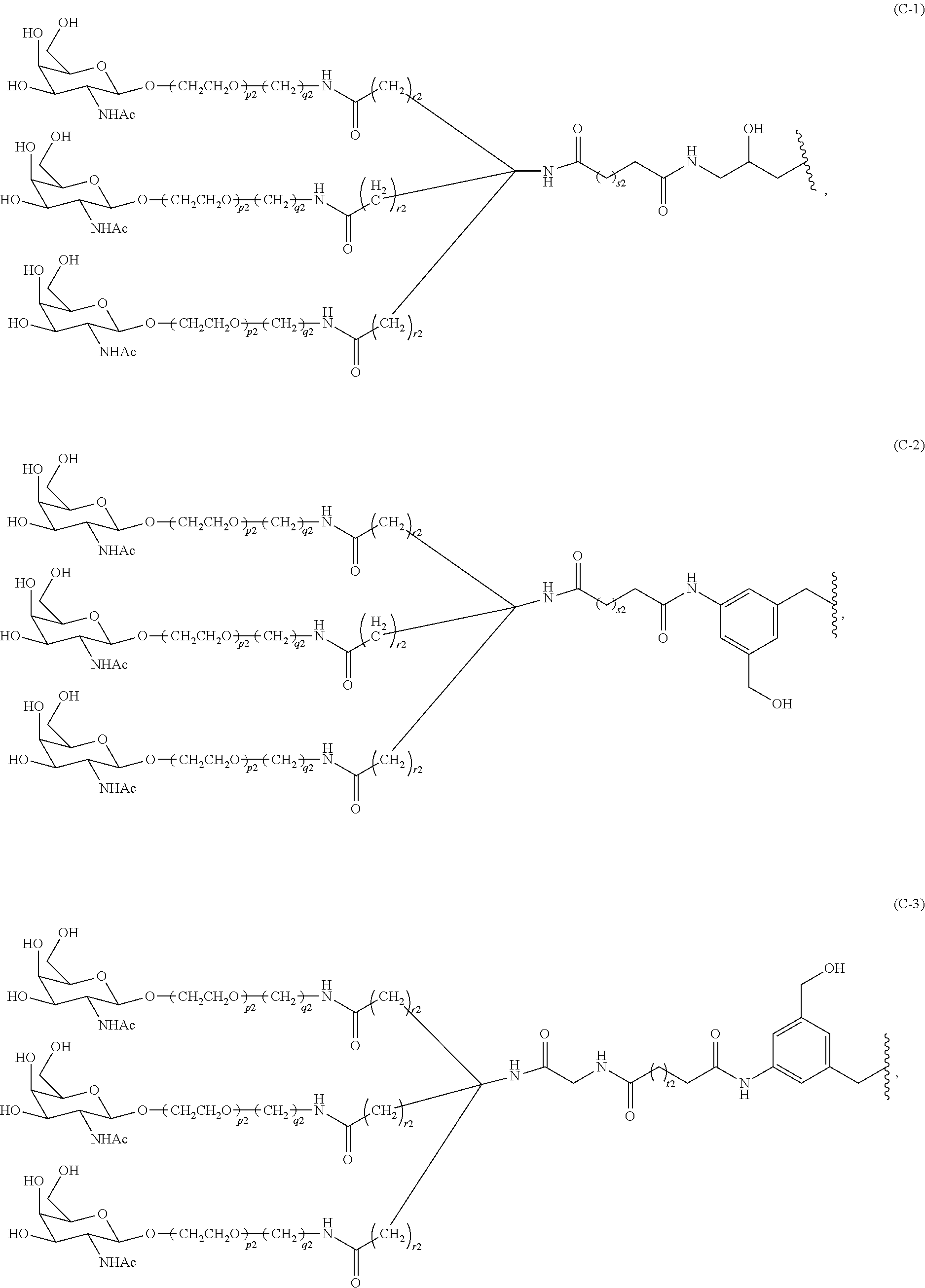

or a pharmaceutically acceptable salt thereof,
   wherein each s2 and t2 is independently an integer from 1 to 12. p2, q2, and r2 are as described above.

Additional suitable ligands related to the above structures of Formula (C) and its subordinates and synthesis thereof are also described in WO2018/191278, entire contents of which are incorporated herein by reference.

In some embodiments, the ligand includes the following structure (D):

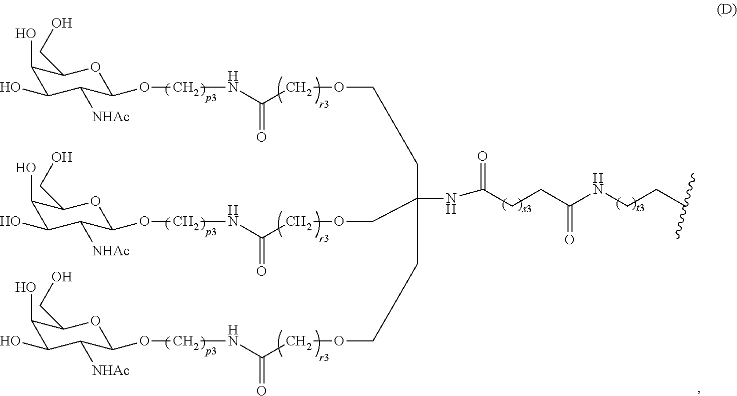

or a pharmaceutically acceptable salt thereof,
wherein each s3 and t3 is independently an integer from 1 to 12. p3 and r3 are as described above.

Additional suitable ligands related to the above structure of Formula (D) and its subordinates and synthesis thereof are also described in WO2014/179620, entire contents of which are incorporated herein by reference.

In some embodiments, the ligand includes the following structure (E-1) to (E-2):

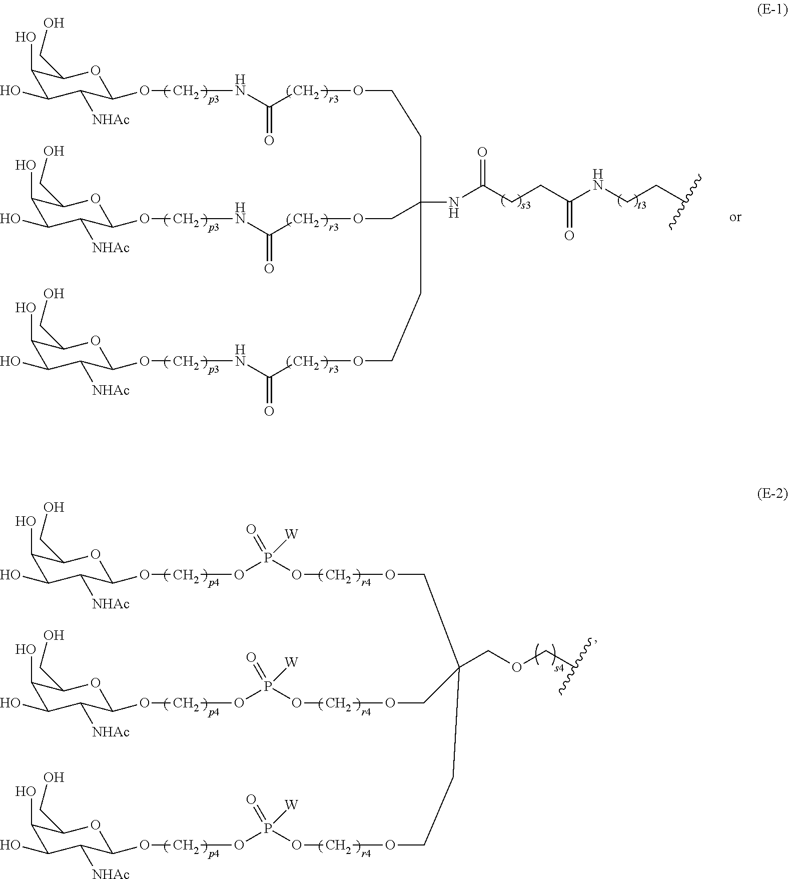

or a pharmaceutically acceptable salt thereof,
wherein each s4 is independently an integer from 1 to 12.
W, p4, and r4 are as described above, a2 is 3 or 4, and [symbol] is an attachment point to the sense strand or the antisense strand, or to a conjugate linker conjugated to the sense strand or the antisense strand.

In some embodiments, at least one of W is —SH. In some embodiments, at least one of W is —OH.

Additional suitable ligands related to the above structure of Formula (E) and its subordinates and synthesis thereof are also described in WO2017/174657, entire contents of which are incorporated herein by reference.

In some embodiments, the ligand includes the following structures:

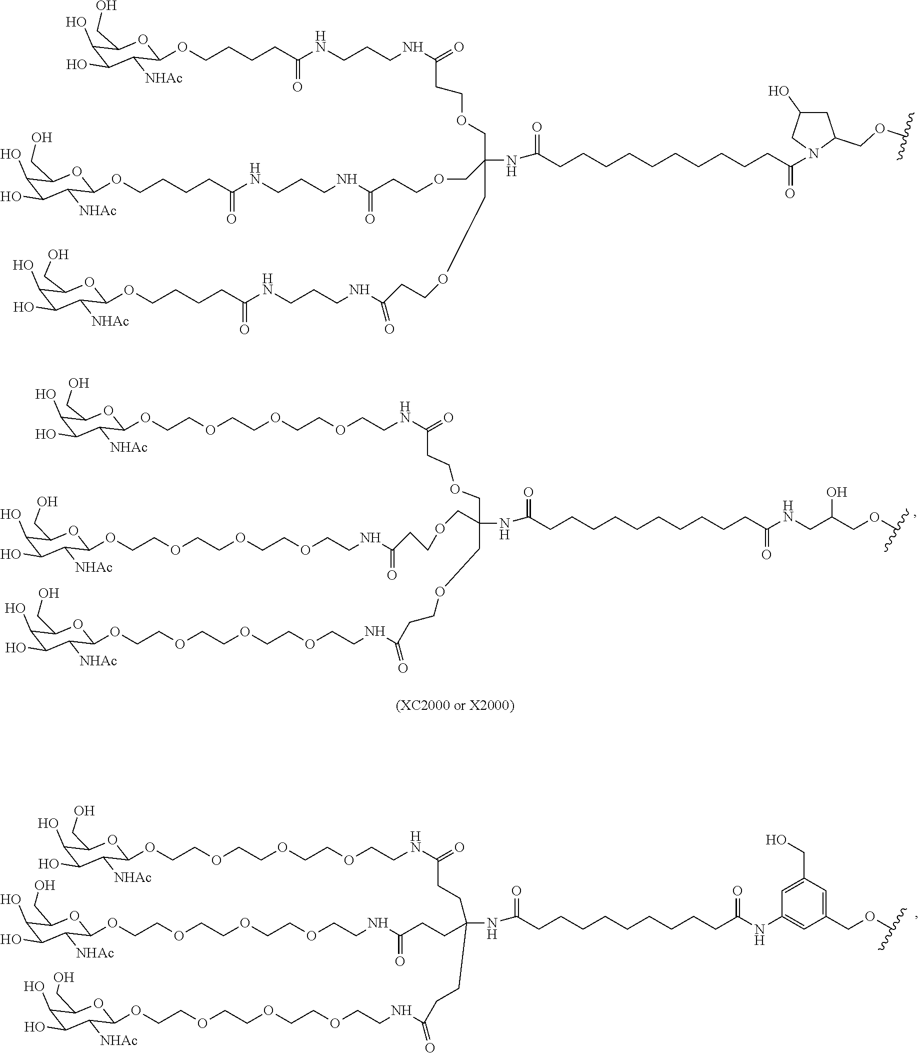

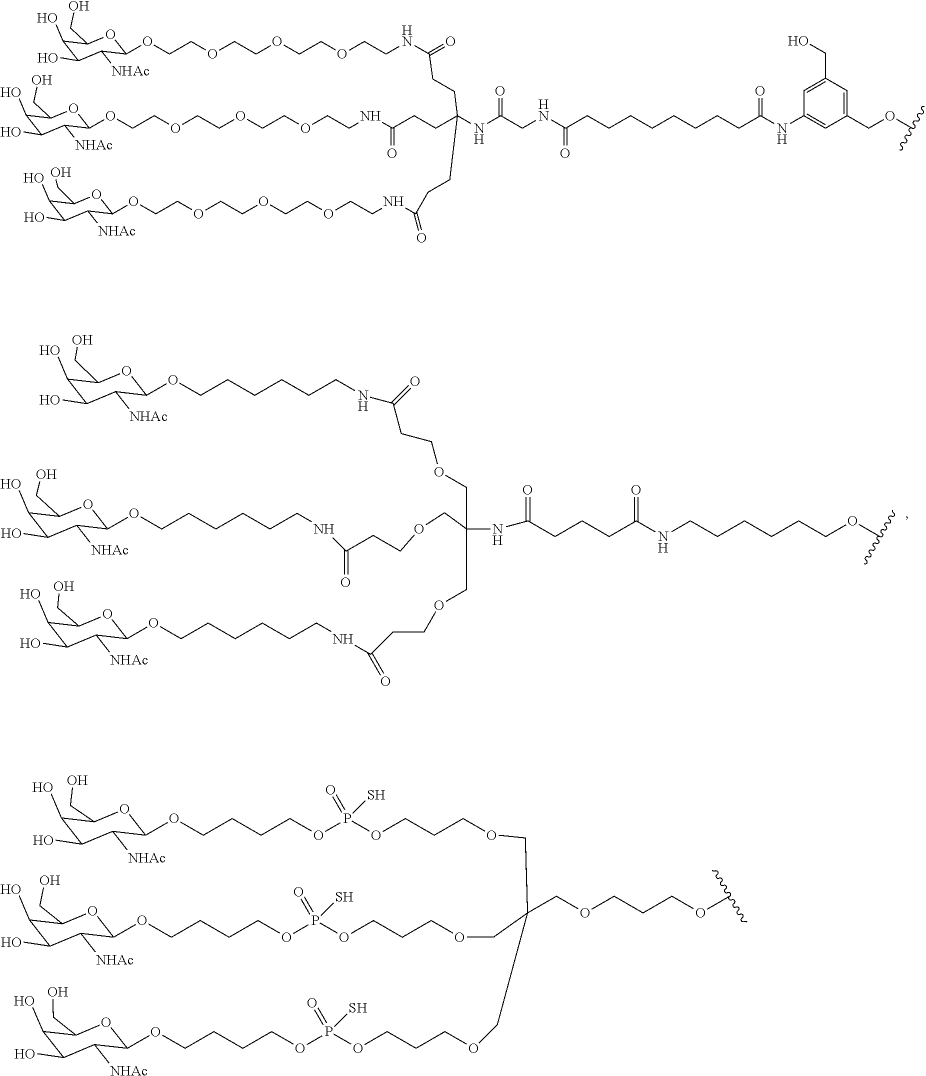 [SMILES: *OCc1cc(CO)cc(NC(=O)CCCCCCCCC(=O)NCC(=O)NC(CCC(=O)NCCOCCOCCOCCOC2OC(CO)C(O)C(O)C2NC(C)=O)(CCC(=O)NCCOCCOCCOCCOC2OC(CO)C(O)C(O)C2NC(C)=O)CCC(=O)NCCOCCOCCOCCOC2OC(CO)C(O)C(O)C2NC(C)=O)c1 ; *OCCCCCCNC(=O)CCCC(=O)NC(COCCC(=O)NCCCCCCOC1OC(CO)C(O)C(O)C1NC(C)=O)(COCCC(=O)NCCCCCCOC1OC(CO)C(O)C(O)C1NC(C)=O)COCCC(=O)NCCCCCCOC1OC(CO)C(O)C(O)C1NC(C)=O ; *OCCCOCC(COCCCOP(=O)(S)OCCCCOC1OC(CO)C(O)C(O)C1NC(C)=O)(COCCCOP(=O)(S)OCCCCOC1OC(CO)C(O)C(O)C1NC(C)=O)COCCCOP(=O)(S)OCCCCOC1OC(CO)C(O)C(O)C1NC(C)=O]

or a pharmaceutically acceptable salt thereof,
wherein [symbol] is an attachment point to the sense strand or the antisense strand, or to a conjugate linker conjugated to the sense strand or the antisense strand.

In some embodiments, the ligand has the following structure:

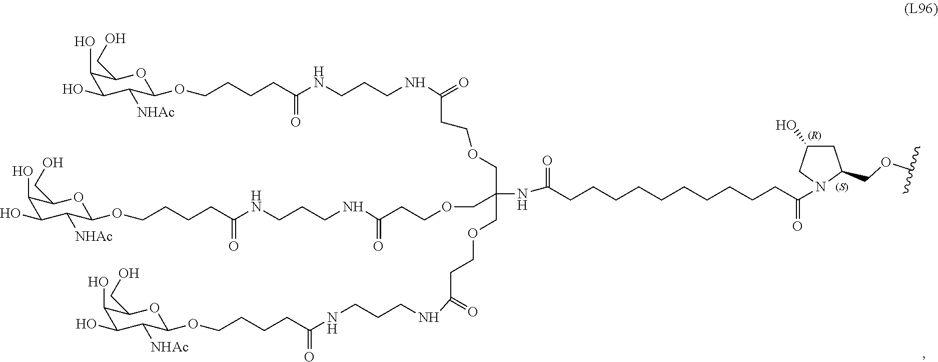

or a pharmaceutically acceptable salt thereof,
wherein [symbol] is an attachment point to the sense strand or the antisense strand, or to a conjugate linker conjugated to the sense strand or the antisense strand.

In some embodiments, the ligand has the following structure:

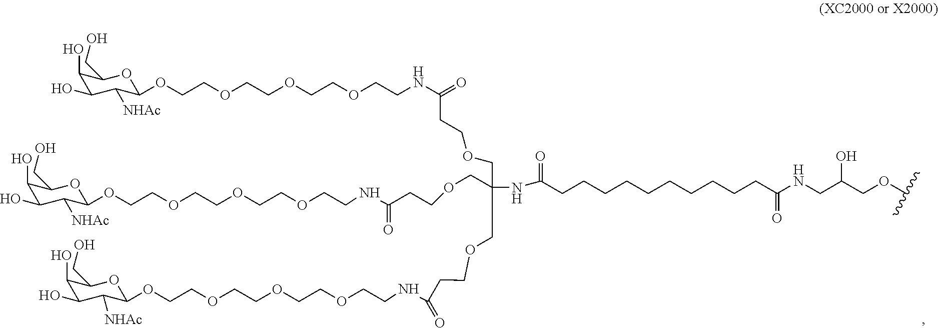

or a pharmaceutically acceptable salt thereof,
wherein [symbol] is an attachment point to the sense strand or the antisense strand, or to a conjugate linker conjugated to the sense strand or the antisense strand.

In certain aspects, the ligand has a structure of Formula (F):

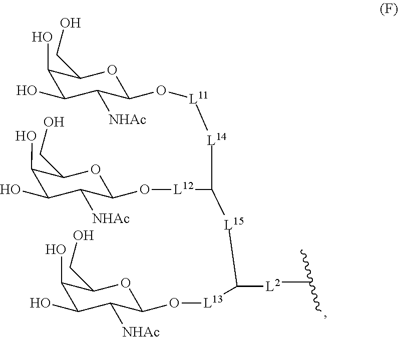

or a pharmaceutically acceptable salt thereof,
   wherein:
   each L¹¹, L¹², L¹³, L¹⁴, and L¹⁵ is an independently a linker;
   L² is a linker as described above;
   [symbol] is an attachment point to the sense strand or the antisense strand, or to a conjugate linker conjugated to the sense strand or the antisense strand.

In some embodiments, each L¹¹ is independently a covalent linker having the formula -L^{11A}-L^{11B}-L^{11C}-L^{11D}-L^{11E}-. Each L^{11A}, L^{11B}, L^{11C}, L^{11D}, and L^{11E} is independently a bond, substituted or unsubstituted alkylene, or a substituted or unsubstituted heteroalkylene. In some embodiments, each L^{11A}, L^{11B}, L^{11C}, L^{11D}, and L^{11E} is independently a bond, substituted or unsubstituted alkylene (e.g., C₁-C₃₀, C₁-C₁₅, or C₁-C₁₂ alkylene), or a substituted or unsubstituted heteroalkylene (e.g., 2 to 30, 2 to 15 membered, or 2 to 12 membered heteroalkylene. In some embodiments, each L^{11A}, L^{11B}, L^{11C}, L^{11D}, and L^{11E} is independently a bond, unsubstituted C₁₁-C₁₁₂ alkylene, —NHC(O)—, —C(O)NH—, —(CH₂)ₐ₁₁—O—, —O—(CH₂)ₐ₁₁—, —(CH₂CH₂O)_{b11}—, or —(OCH₂CH₂)_{b11}—, and each a11 or b11 is independently an integer from 0 to 12.

In some embodiments, each L¹² is independently a covalent linker having the formula -L^{12A}-L^{12B}-L^{12C}-L^{12D}-L^{12E}-. Each L^{12A}, L^{12B} L^{12C}, L^{12D}, and L^{12E} is independently a bond, substituted or unsubstituted alkylene, or a substituted or unsubstituted heteroalkylene. In some embodiments, each L^{12A}, L^{12B}, L^{12C}, L^{12D}, and L^{12E} is independently a bond, substituted or unsubstituted alkylene (e.g., C₁-C₃₀, C₁-C₁₅, or C₁-C₁₂ alkylene), or a substituted or unsubstituted heteroalkylene (e.g., 2 to 30, 2 to 15 membered, or 2 to 12 membered heteroalkylene. In some embodiments, each L^{12A}, L^{12B}, L^{12C}, L^{12D}, and L^{12E} is independently a bond, unsubstituted C₁₂-C₁₂₂ alkylene, —NHC(O)—, —C(O)NH—, —(CH₂)ₐ₁₂—O—, —O—(CH₂)ₐ₁₂—, —(CH₂CH₂O)_{b12}—, or —(OCH₂CH₂)_{b12}—, and each a12 or b12 is independently an integer from 0 to 12.

In some embodiments, each L¹³ is independently a covalent linker having the formula -L^{13A}-L^{13B}-L^{13C}-L^{13D}-L^{13E}-. Each L^{13A}, L^{13B}, L^{13C}, L^{13D}, and L^{13E} is independently a bond, substituted or unsubstituted alkylene, or a substituted or unsubstituted heteroalkylene. In some embodiments, each L^{13A}, L^{13B}, L^{13C}, L^{13D}, and L^{13E} is independently a bond, substituted or unsubstituted alkylene (e.g., C₁-C₃₀, C₁-C₁₅, or C₁-C₁₂ alkylene), or a substituted or unsubstituted heteroalkylene (e.g., 2 to 30, 2 to 15 membered, or 2 to 12 membered heteroalkylene. In some embodiments, each L^{13A}, L^{13B}, L^{13C}, L^{13D}, and L^{13E} is independently a bond, unsubstituted C₁ₐ₁₄-C₁₂ alkylene, —NHC(O)—, —C(O)NH—, —(CH₂)ₐ₁₃—O—, —O—(CH₂)ₐ₁₃—, —(CH₂CH₂O)_{b13}—, or —(OCH₂CH₂)_{b13}—, and each a13 or b13 is independently an integer from 0 to 12.

In some embodiments, L¹⁴ has the formula -L^{14A}-L^{14B}-L^{14C}-L^{14D}-L^{14E}-. Each L^{14A}L^{14B}, L^{14C}, L^{14D}, and L^{14E} is independently a bond, substituted or unsubstituted alkylene, or a substituted or unsubstituted heteroalkylene. In some embodiments, each L^{14A}, L^{14B}, L^{14C}, L^{14D}, and L^{14E} is independently a bond, unsubstituted C₁-C₁₂ alkylene, —NHC(O)—, —C(O)NH—, —(CH₂)ₐ₁₄—O—, —O—(CH₂)ₐ₁₄—, —(CH₂CH₂O)_{b14}—,or —(OCH₂CH₂)_{b14}—, and each a14 or b14 is independently an integer from 0 to 12. In some embodiments, L¹⁴ is a bond. In some embodiments, L¹⁴ is unsubstituted C₁-C₁₂ alkylene. In some embodiments, L¹⁴ is —C(O)NH—(CH₂)_{z1}, or —NHC(O)—(CH₂)_{z1} wherein z1 is an integer from 0 to 12.

In some embodiments, L¹⁵ has the formula -L^{15A}-L^{15B}-L^{15C}-L^{15D}-L^{15E}-. Each L^{15A}, L^{15B}, L^{15C}, L^{15D}, and L^{15E} is independently a bond, substituted or unsubstituted alkylene, or a substituted or unsubstituted heteroalkylene. In some embodiments, each L^{15A}, L^{15B}, L^{15C}, L^{15D}, and L^{15E} is independently a bond, unsubstituted C₁-C₁₂ alkylene, —NHC(O)—, —C(O)NH—, —(CH₂)ₐ₁₅—O—, —O—(CH₂)ₐ₁₅—, —(CH₂CH₂O)_{b15}—,or —(OCH₂CH₂)_{b15}—, and each a15 or b15 is independently an integer from 0 to 12. In some embodiments, L¹⁵ is unsubstituted C₁-C₁₂ alkylene. In some embodiments, L¹⁵ is —C(O)NH—(CH₂)_{z2}, or —NHC(O)—(CH₂)_{z2} wherein z2 is an integer from 0 to 12. In some embodiments, L¹⁵ is —C(O)NH— or —NHC(O)—.

In certain aspects, the ligand includes the following structure:

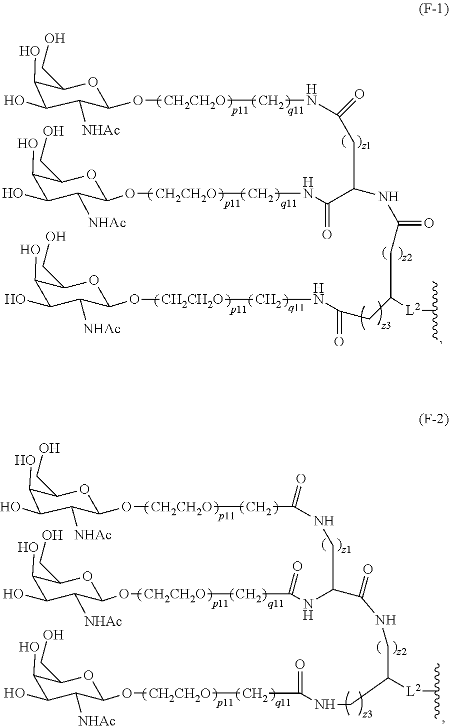

or a pharmaceutically acceptable salt thereof,
wherein:
L² is as described above;
each p11 and q11 is independently an integer from 0 to 12;
each z1, z2, and z3 is independently an integer of 0 to 12; and
[symbol] is an attachment point to the sense strand or the antisense strand, or to a conjugate linker conjugated to the sense strand or the antisense strand.

In some embodiments, the ligand includes the following structures (F-1-a) to (F-1-c):

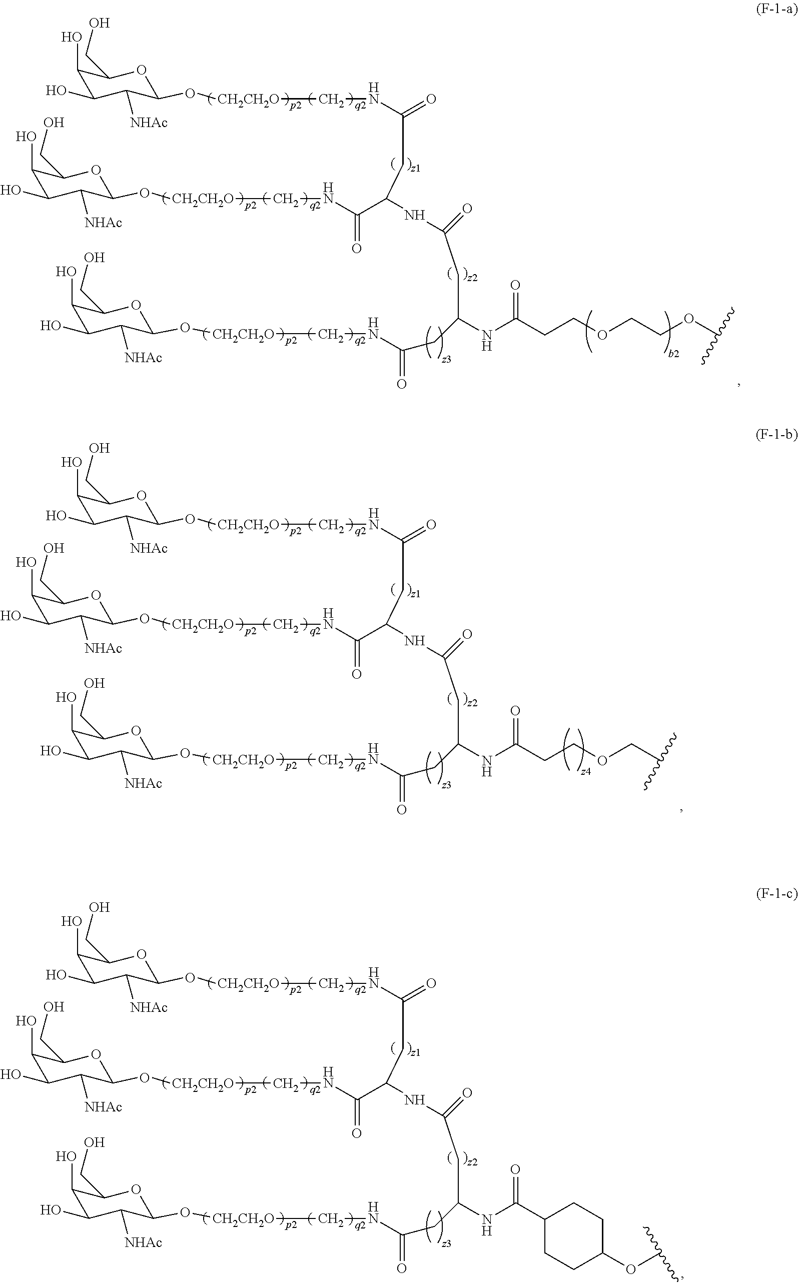

or a pharmaceutically acceptable salt thereof,
   wherein W, p2, q2, z1, z2, and z3 are as described above. z4 is an integer from 0 to 10.

In some embodiments, the ligand includes the following structures (F-2-a) to (F-2-c):

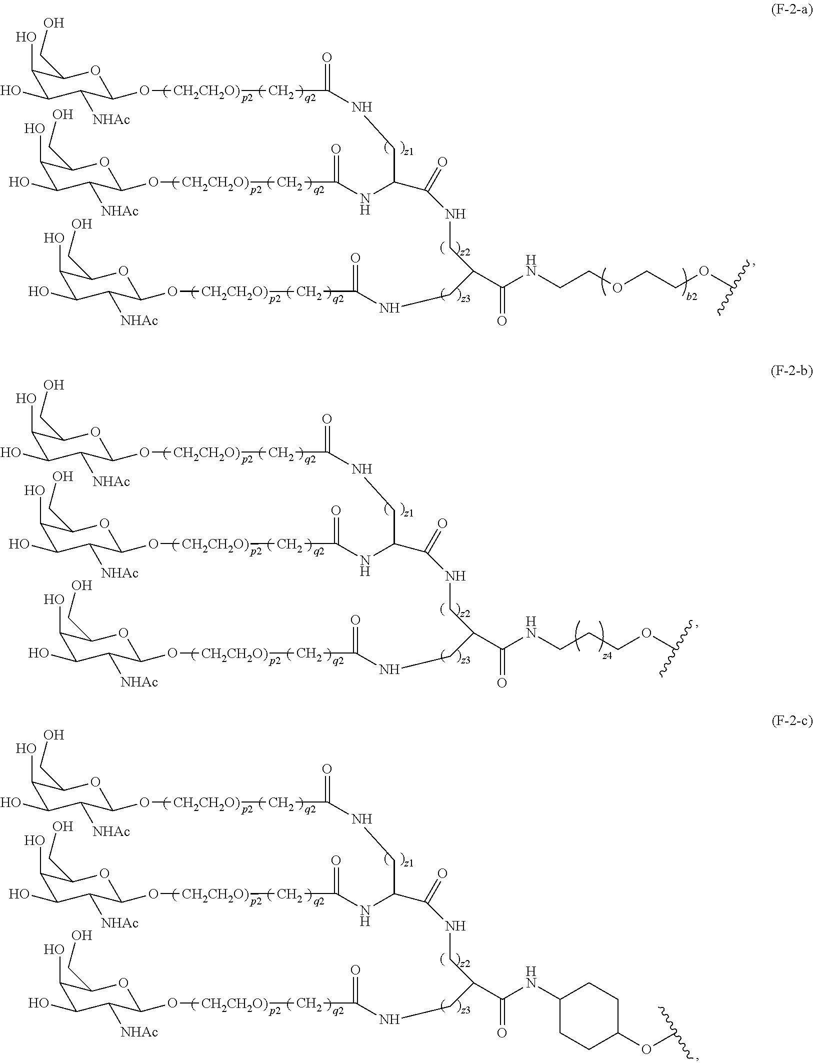

or a pharmaceutically acceptable salt thereof,
wherein W, p2, q2, z1, z2 and z3 are as described above. z4 is an integer from 0 to 10.

In some embodiments, z1 is 0. In some embodiments, z1 is 1. In some embodiments, z1 is 2. In some embodiments, z1 is 3. In some embodiments, z1 is 4. In some embodiments, z2 is 0. In some embodiments, z2 is 1. In some embodiments, z2 is 2. In some embodiments, z2 is 3. In some embodiments, z2 is 4. In some embodiments, z3 is 0. In some embodiments, z3 is 1. In some embodiments, z3 is 2. In some embodiments, z3 is 3. In some embodiments, z3 is 4.

In some embodiments, the ligand includes the following structure:

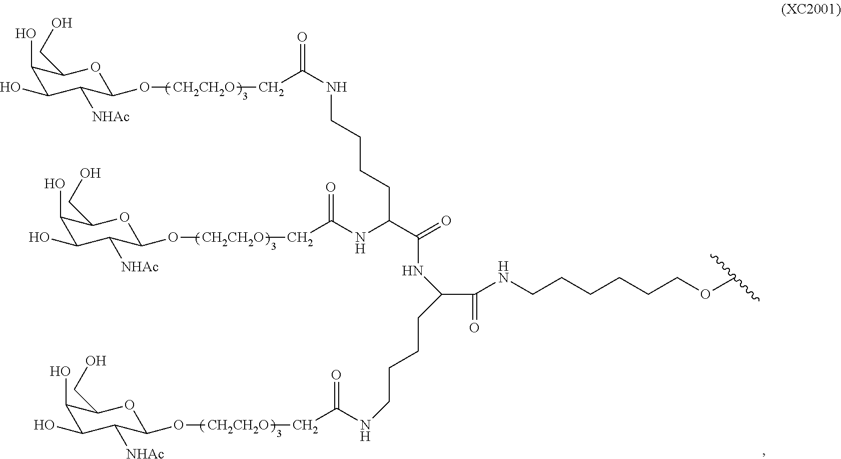

or a pharmaceutically acceptable salt thereof.

Additional suitable ligands related to the above structures of Formula (F) and its subordinates and synthesis thereof are also described in WO2011/104169 and WO2008/022309, entire contents of which are incorporated herein by reference.

In some embodiments, the ligand is coupled or conjugated to the 3′ end of the sense strand. In some embodiments, the ligand is coupled or conjugated to the 5′ end of the sense strand. In some embodiments, the ligand is coupled or conjugated to the 3′ end of the antisense strand. In some embodiments, the ligand is coupled or conjugated to the 5′ end of the antisense strand. In some embodiments, two ligands may be coupled to both sense strand and antisense strand. In some embodiments, the ligand is conjugated to a “non-end” of the sense strand or antisense strand.

In some embodiments, the ligands may be conjugated to the 3′ end of the sense strand and to the 3′ end of the antisense strand. In some embodiments, the ligands may be conjugated to the 5′ end of the sense strand and to the 3′ end of the antisense strand. In some embodiments, the ligands may be conjugated to a non-end of the sense strand and to the 3′ end of the antisense strand. In some embodiments, the ligands may be conjugated to the 3′ end of the sense strand and to a non-end of the antisense strand. In some embodiments, the ligands may be conjugated to the 5′ end of the sense strand and to a non-end of the antisense strand. In some embodiments, the ligands may be conjugated to a non-end of the sense strand and to a non-end of the antisense strand (e.g., nucleobases).

In some embodiments, the dsRNAi agent includes the following structure:

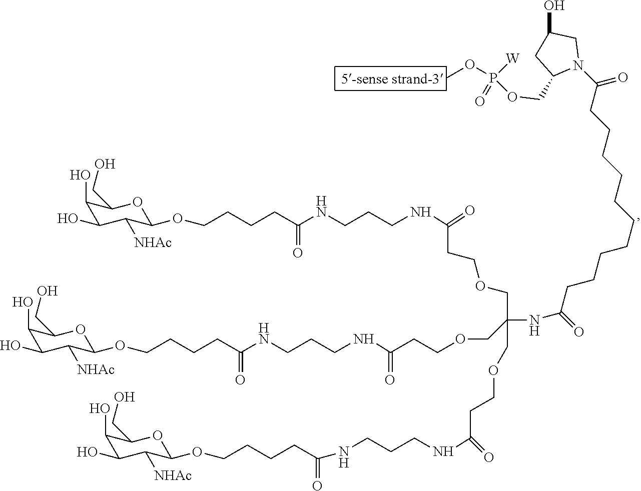

or a pharmaceutically acceptable salt thereof,
wherein W is —OH or —SH.

In some embodiments, the dsRNAi agent includes the following structure:

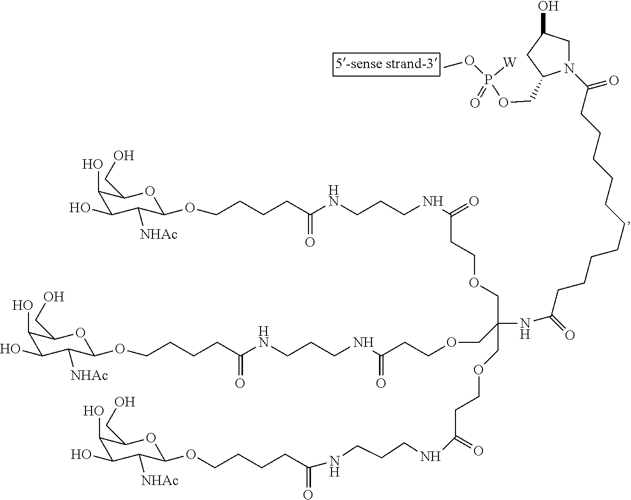

or a pharmaceutically acceptable salt thereof,
wherein W is —OH or —SH, and the sense strand includes any one sense strand selected from SEQ ID NOs: 1-405, 812-1052, 1294-1297, 1434-1440, 1448-1462, and 1481-1482.

In some embodiments, the RNAi agent includes the following structure:

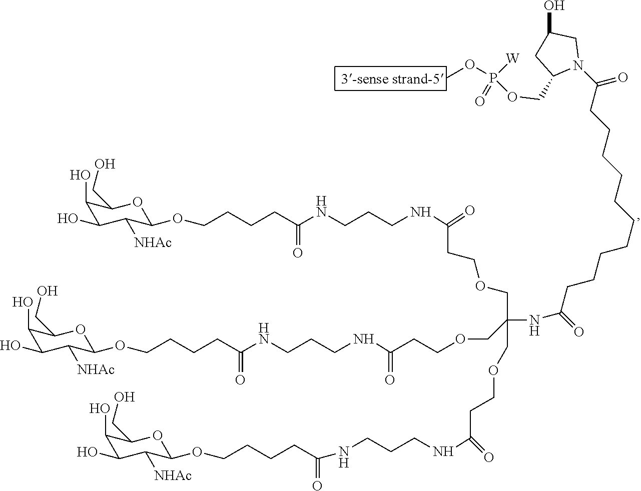

or a pharmaceutically acceptable salt thereof,
wherein W is —OH or —SH.

In some embodiments, the RNAi agent includes the following structure:

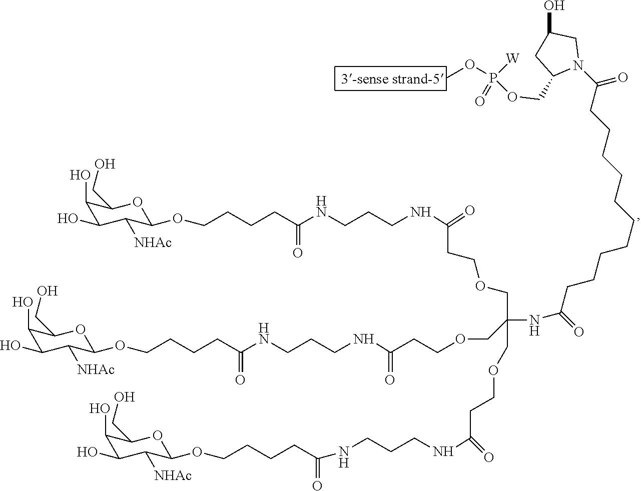

or a pharmaceutically acceptable salt thereof,
wherein W is —OH or —SH, and the sense strand includes any one sense strand selected from SEQ ID NOs: 1-405, 812-1052, 1294-1297, 1434-1440, 1448-1462, and 1481-1482.

In some embodiments, the RNAi agent includes the following structure:

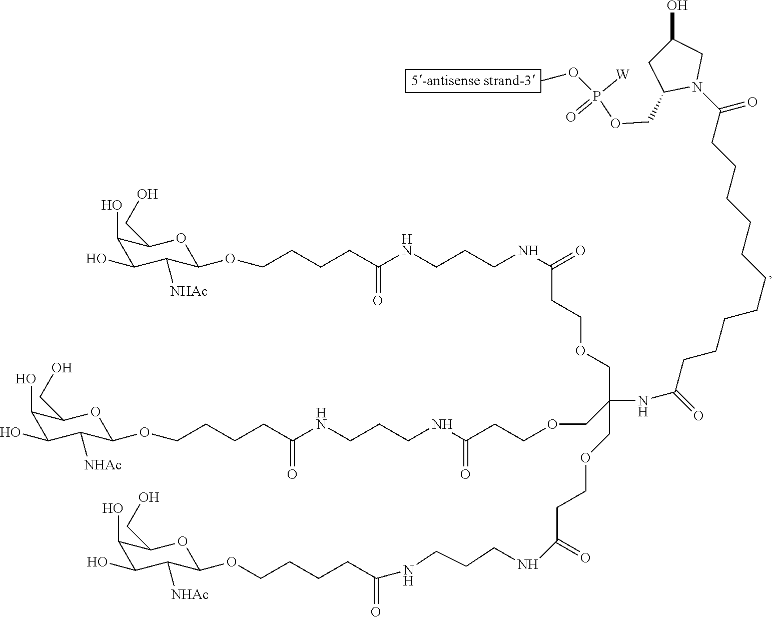

or a pharmaceutically acceptable salt thereof,
wherein W is —OH or —SH.

In some embodiments, the RNAi agent includes the following structure:

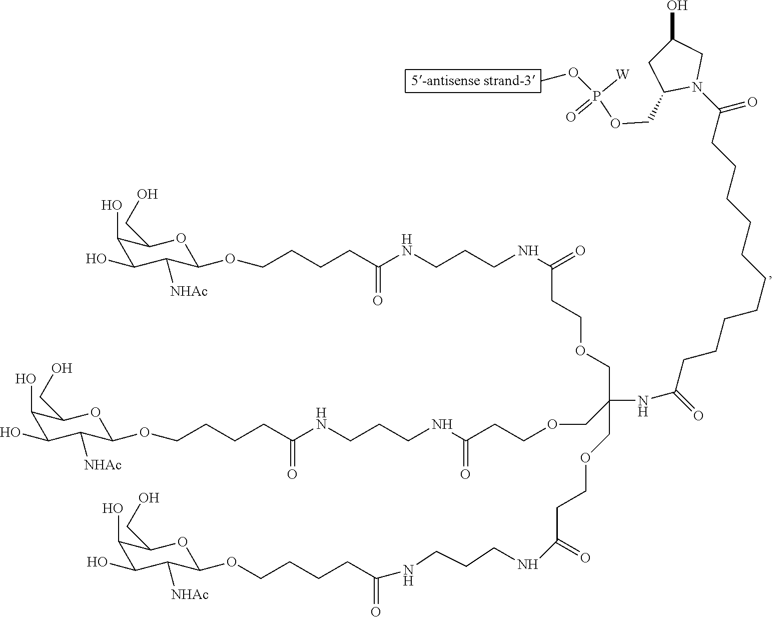

or a pharmaceutically acceptable salt thereof,
wherein W is —OH or —SH, and the antisense strand includes any antisense strand selected from SEQ ID NOs: 406-810, 1053-1293, 1298-1301, 1441-1447, and 1463-1477.

In some embodiments, the RNAi agent includes the following structure:

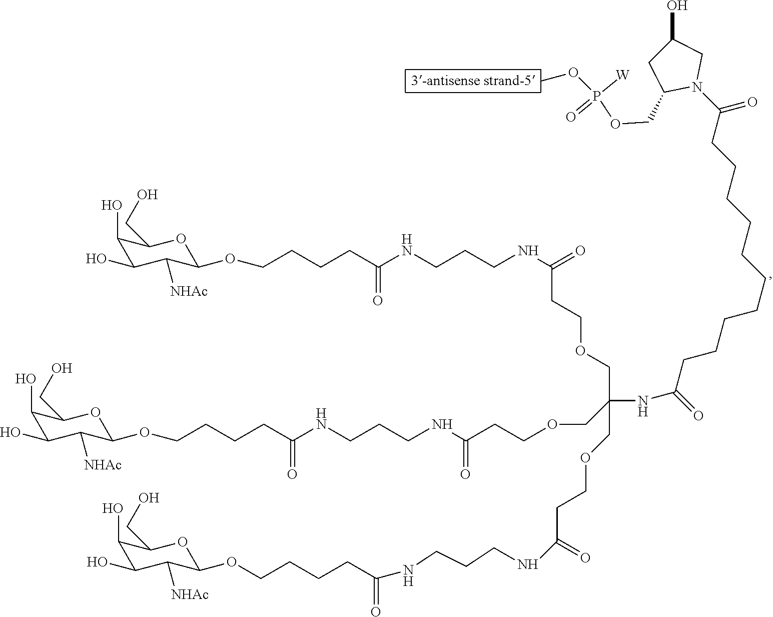

or a pharmaceutically acceptable salt thereof,
wherein W is —OH or —SH.

In some embodiments, the RNAi agent includes the following structure:

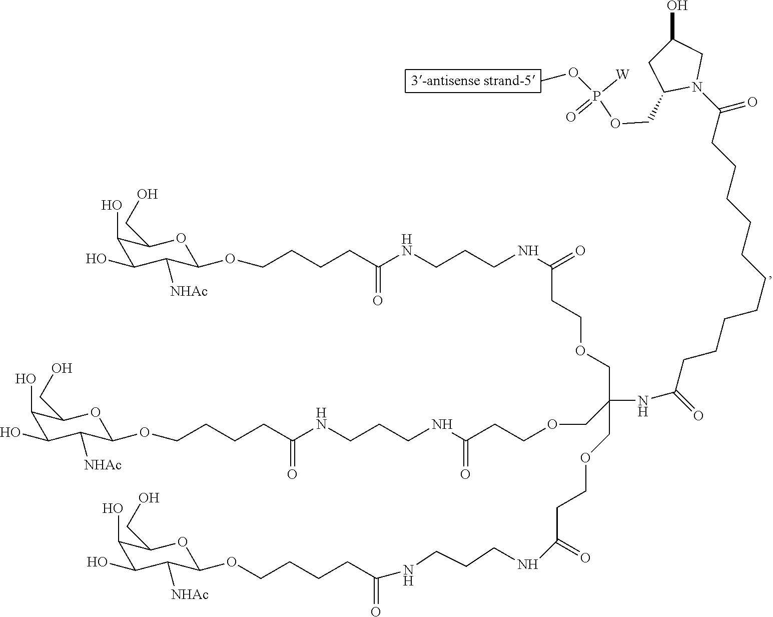

or a pharmaceutically acceptable salt thereof,
wherein W is —OH or —SH, and the antisense strand includes any antisense strand selected from SEQ ID NOs: 406-810, 1053-1293, 1298-1301, 1441-1447, and 1463-1477.

In some embodiments, the RNAi agent includes the following structure:

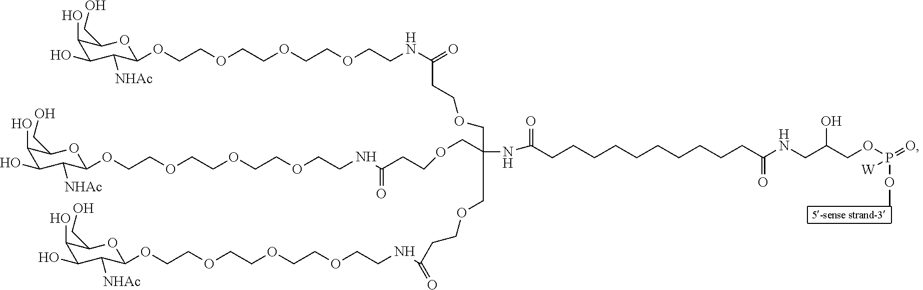

or a pharmaceutically acceptable salt thereof,
wherein W is —OH or —SH.

In some embodiments, the RNAi agent includes the following structure:

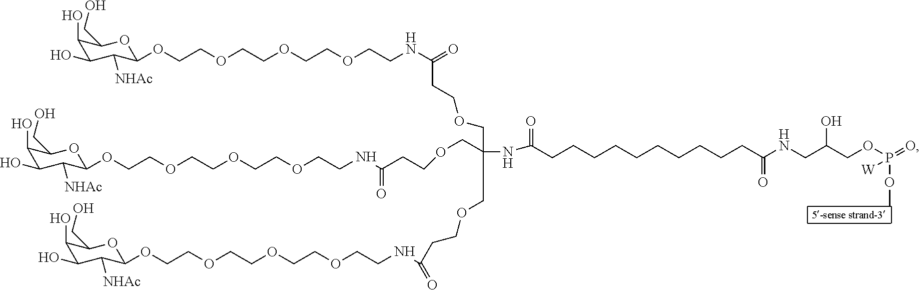

or a pharmaceutically acceptable salt thereof,
   wherein W is —OH or —SH, and the sense strand includes any one sense strand selected from SEQ ID NOs: 1-405, 812-1052, 1294-1297, 1434-1440, 1448-1462, and 1481-1482.

In some embodiments, the RNAi agent includes the following structure:

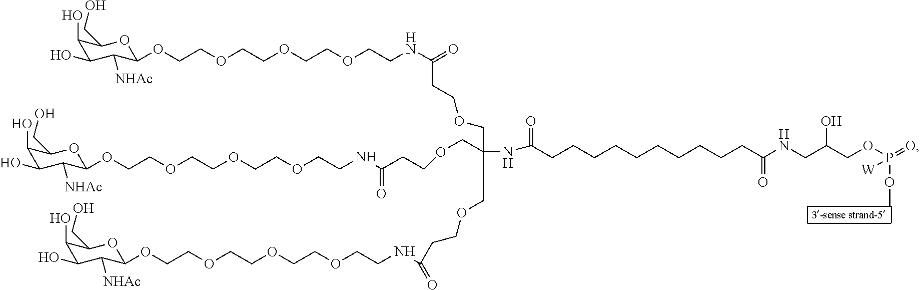

or a pharmaceutically acceptable salt thereof,
   wherein W is —OH or —SH.

In some embodiments, the RNAi agent includes the following structure:

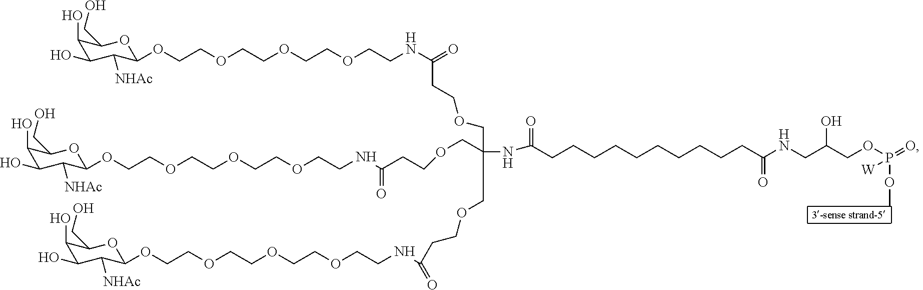

or a pharmaceutically acceptable salt thereof,
   wherein W is —OH or —SH, and the sense strand includes any one sense strand selected from SEQ ID NOs: 1-405, 812-1052, 1294-1297, 1434-1440, 1448-1462, and 1481-1482.

In some embodiments, the RNAi agent includes the following structure:

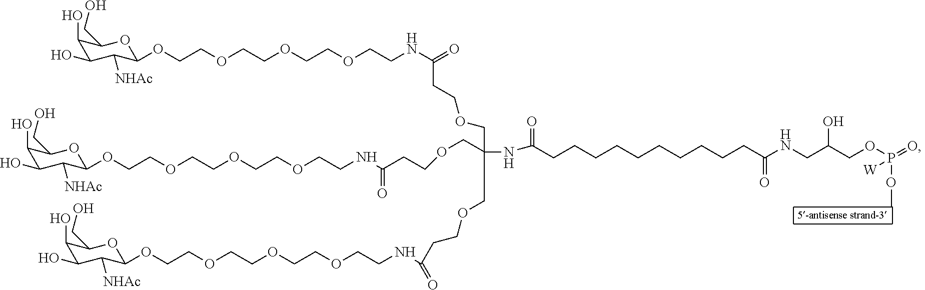

or a pharmaceutically acceptable salt thereof,
   wherein W is —OH or —SH.

In some embodiments, the RNAi agent includes the following structure:

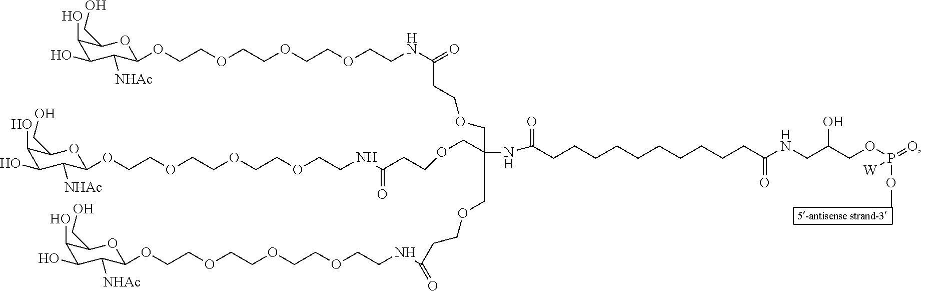

or a pharmaceutically acceptable salt thereof,
   wherein W is —OH or —SH, and the antisense strand includes any antisense strand selected from SEQ ID NOs: 406-810, 1053-1293, 1298-1301, 1441-1447, and 1463-1477.

In some embodiments, the RNAi agent includes the following structure:

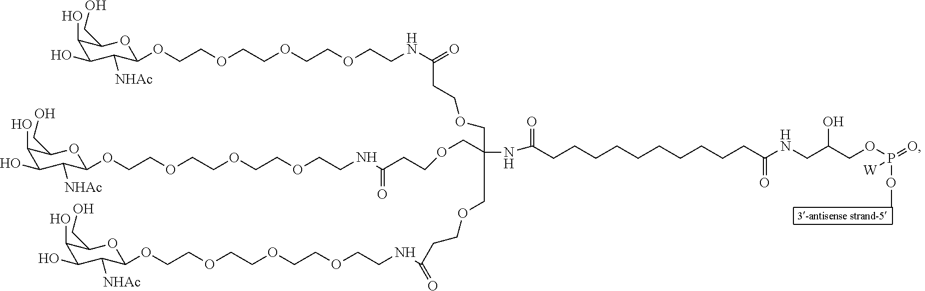

or a pharmaceutically acceptable salt thereof,
   wherein W is —OH or —SH.

In some embodiments, the RNAi agent includes the following structure:

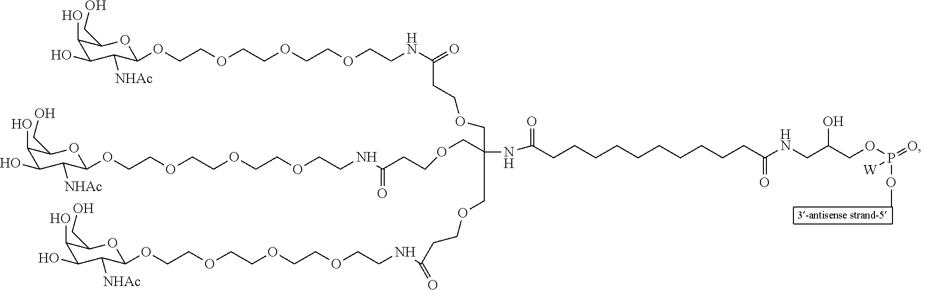

or a pharmaceutically acceptable salt thereof,
   wherein W is —OH or —SH, and the antisense strand includes any antisense strand selected from SEQ ID NOs: 406-810, 1053-1293, 1298-1301, 1441-1447, and 1463-1477.

In some embodiments, the RNAi agent includes the following structure:

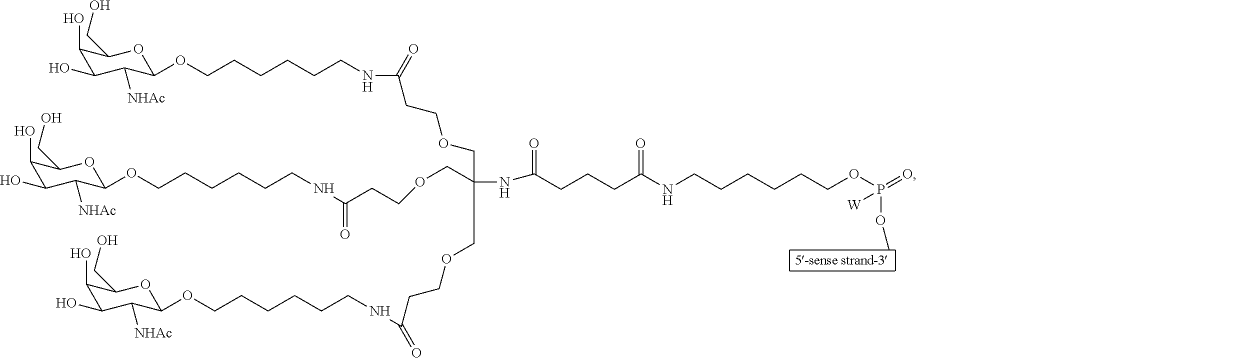

or a pharmaceutically acceptable salt thereof,
   wherein W is —OH or —SH.

In some embodiments, the RNAi agent includes the following structure:

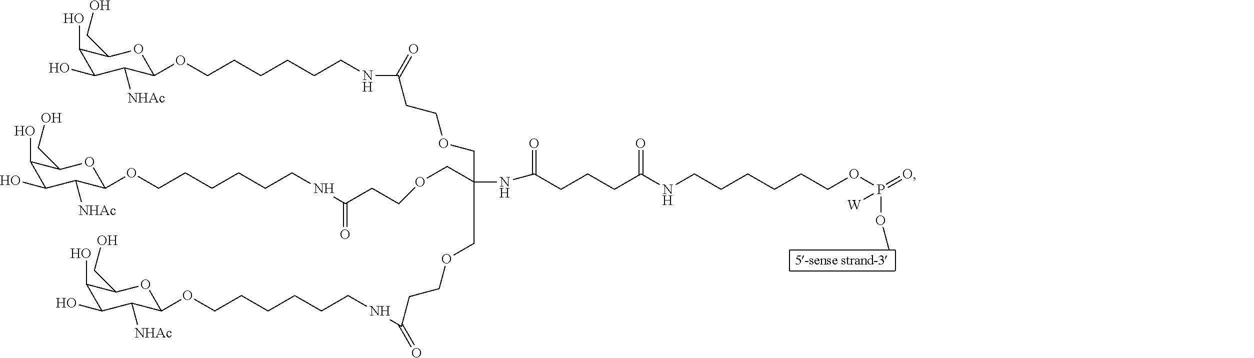

or a pharmaceutically acceptable salt thereof,
   wherein W is —OH or —SH, and the sense strand includes any one sense strand selected from SEQ ID NOs: 1-405, 812-1052, 1294-1297, 1434-1440, 1448-1462, and 1481-1482.

In some embodiments, the RNAi agent includes the following structure:

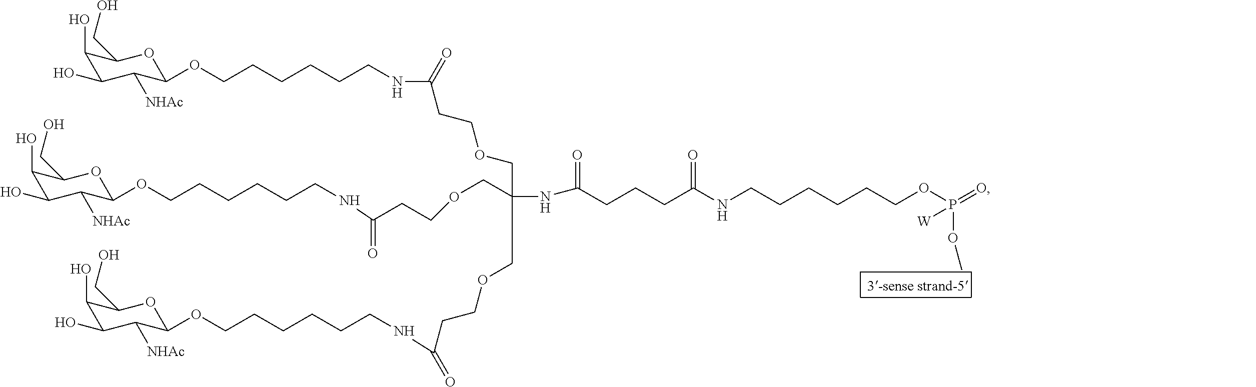

or a pharmaceutically acceptable salt thereof,
   wherein W is —OH or —SH.

In some embodiments, the RNAi agent includes the following structure:

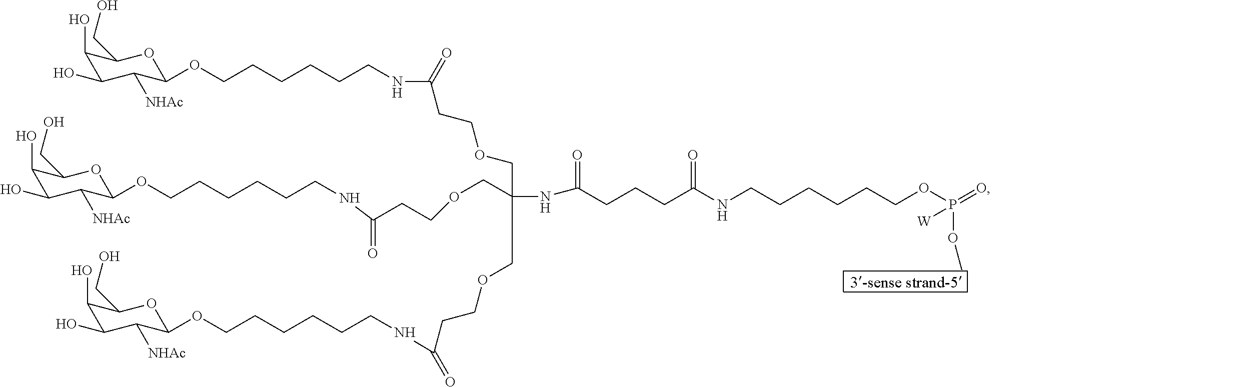

or a pharmaceutically acceptable salt thereof,
   wherein W is —OH or —SH, and the sense strand includes any one sense strand selected from SEQ ID NOs: 1-405, 812-1052, 1294-1297, 1434-1440, 1448-1462, and 1481-1482.

In some embodiments, the RNAi agent includes the following structure:

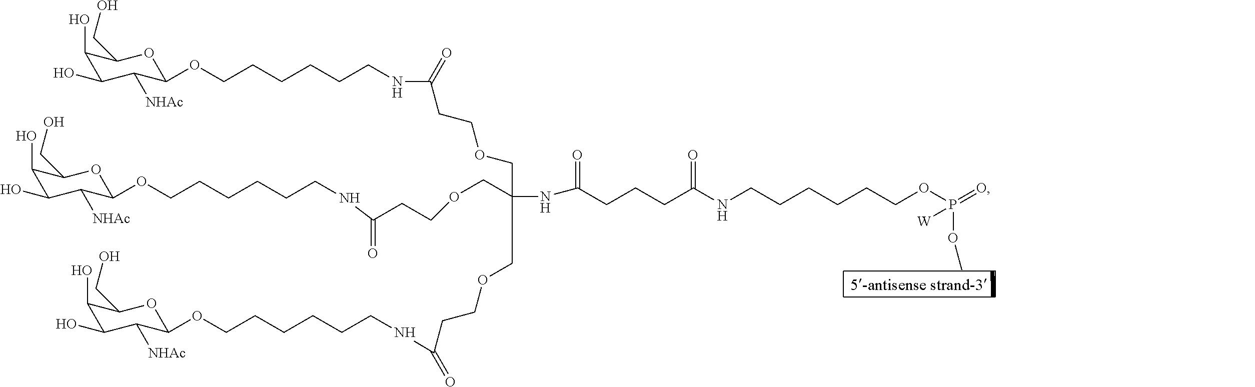

or a pharmaceutically acceptable salt thereof,
   wherein W is —OH or —SH.

In some embodiments, the RNAi agent includes the following structure:

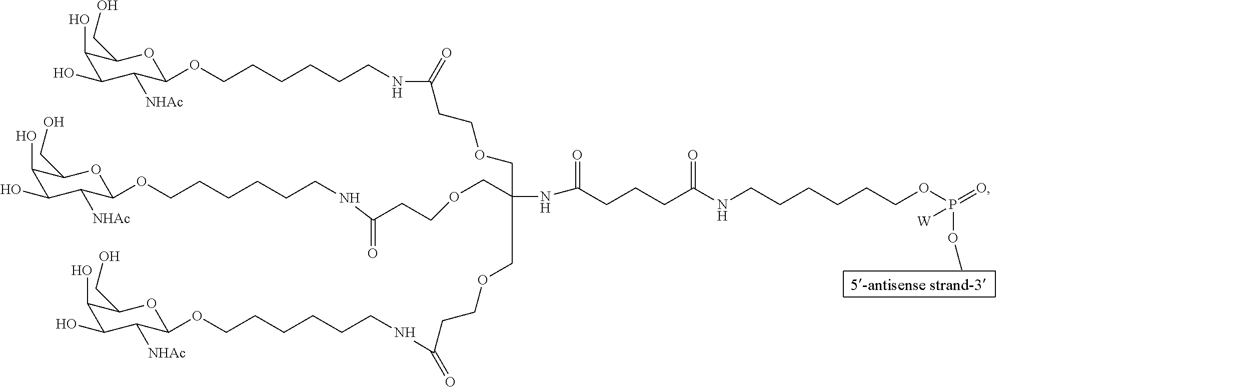

or a pharmaceutically acceptable salt thereof,
   wherein W is —OH or —SH, and the antisense strand includes any antisense strand selected from SEQ ID NOs: 406-810, 1053-1293, 1298-1301, 1441-1447, and 1463-1477.

In some embodiments, the RNAi agent includes the following structure:

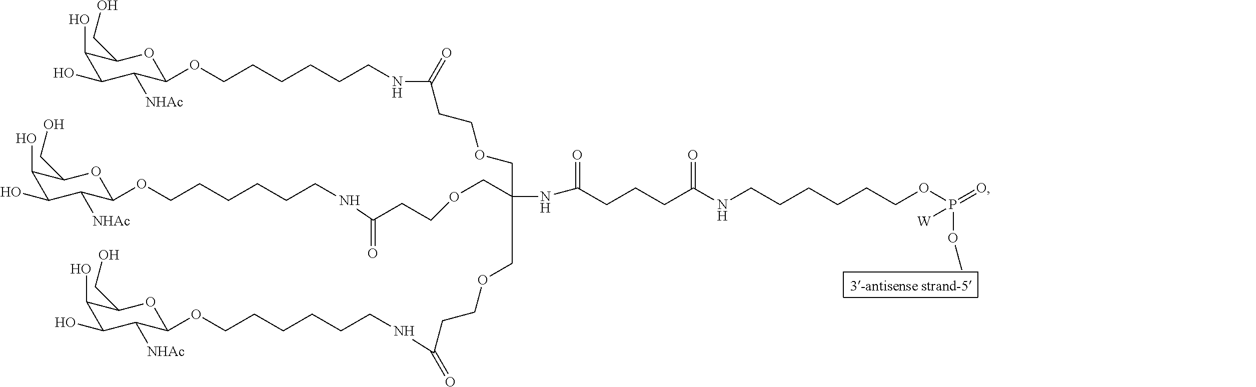

or a pharmaceutically acceptable salt thereof,
   wherein W is —OH or —SH.

In some embodiments, the RNAi agent includes the following structure:

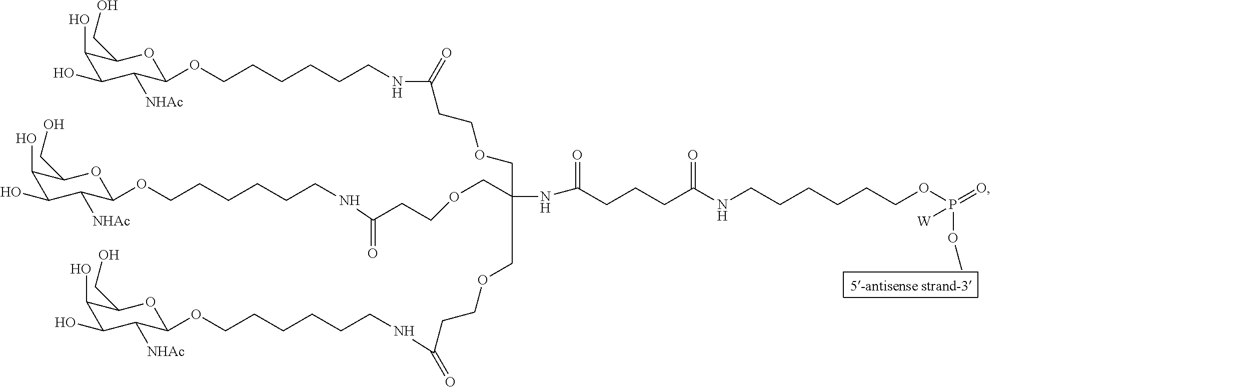

or a pharmaceutically acceptable salt thereof,
   wherein W is —OH or —SH, and the antisense strand includes any antisense strand selected from SEQ ID NOs: 406-810, 1053-1293, 1298-1301, 1441-1447, and 1463-1477.

In some embodiments, the RNAi agent includes the following structure:

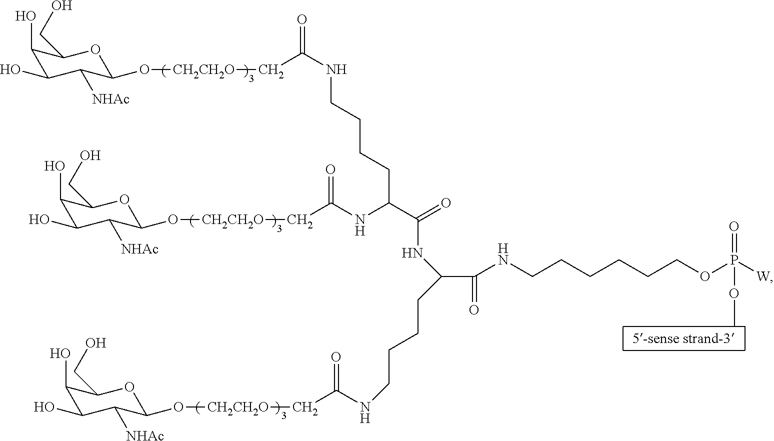

or a pharmaceutically acceptable salt thereof,
   wherein W is —OH or —SH.

In some embodiments, the RNAi agent includes the following structure:

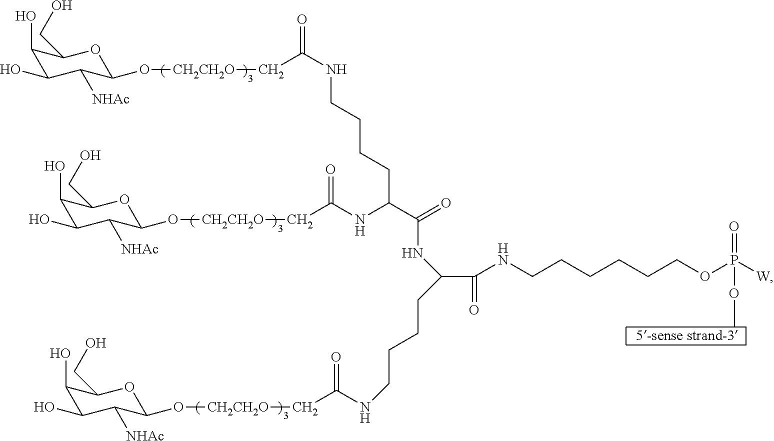

or a pharmaceutically acceptable salt thereof,
   wherein W is —OH or —SH, and the sense strand includes any one sense strand selected from SEQ ID NOs: 1-405, 812-1052, 1294-1297, 1434-1440, 1448-1462, and 1481-1482.

In some embodiments, the RNAi agent includes the following structure:

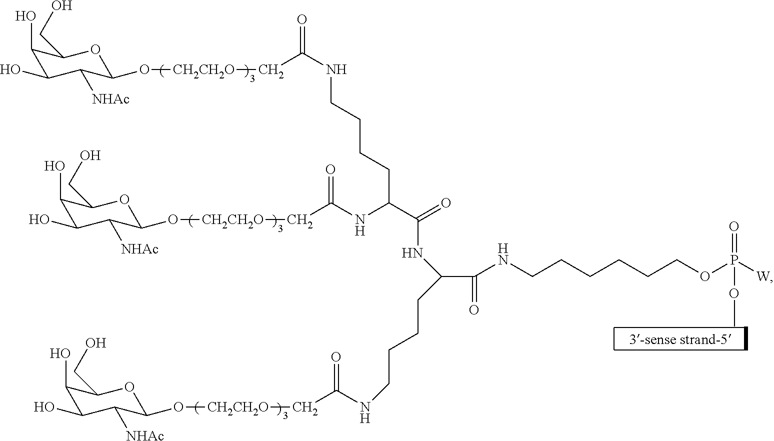

or a pharmaceutically acceptable salt thereof,
   wherein W is —OH or —SH.

In some embodiments, the RNAi agent includes the following structure:

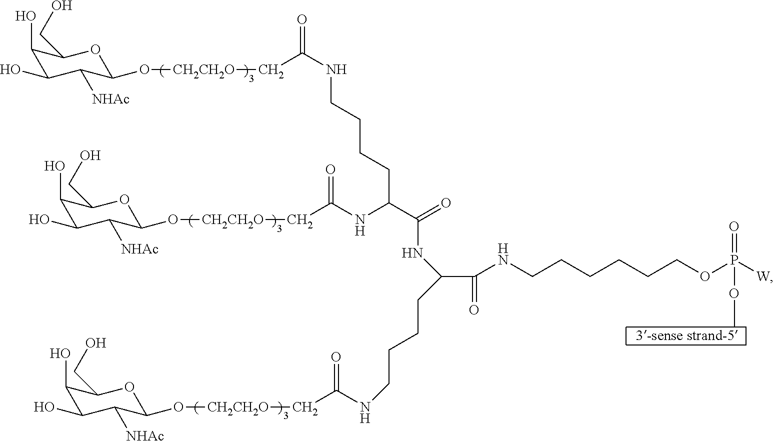

or a pharmaceutically acceptable salt thereof,
   wherein W is —OH or —SH, and the sense strand includes any one sense strand selected from SEQ ID NOs: 1-405, 812-1052, 1294-1297, 1434-1440, 1448-1462, and 1481-1482.

In some embodiments, the RNAi agent includes the following structure:

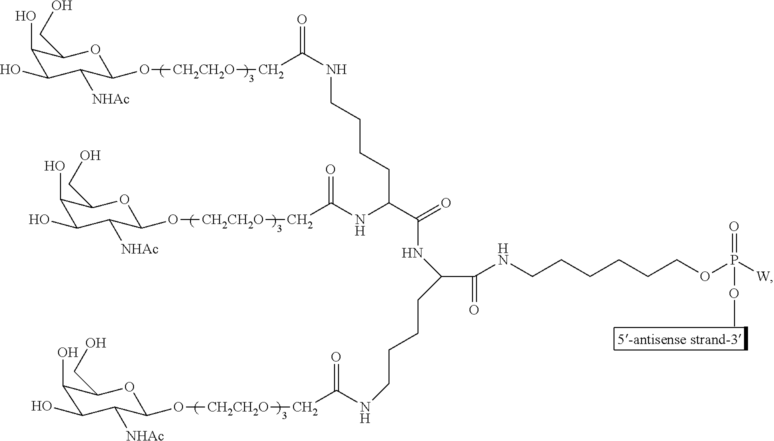

or a pharmaceutically acceptable salt thereof,
   wherein W is —OH or —SH.

In some embodiments, the RNAi agent includes the following structure:

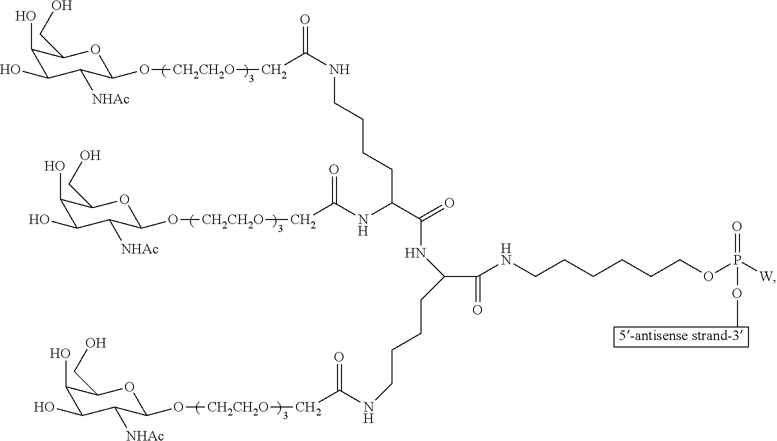

or a pharmaceutically acceptable salt thereof,
   wherein W is —OH or —SH, and the antisense strand includes any antisense strand selected from SEQ ID NOs: 406-810, 1053-1293, 1298-1301, 1441-1447, and 1463-1477.

In some embodiments, the RNAi agent includes the following structure:

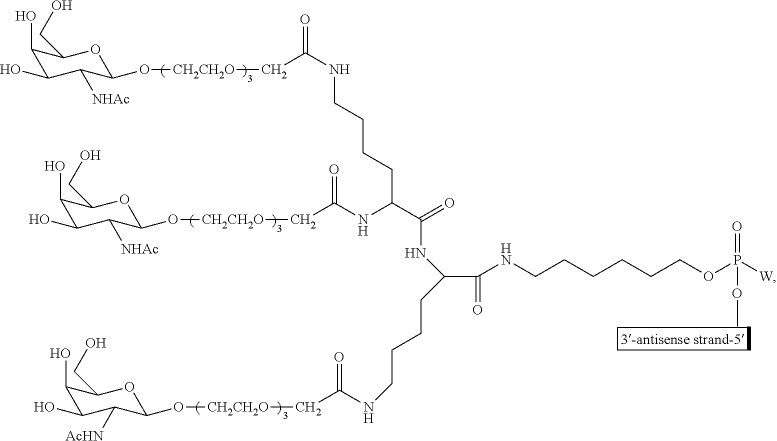

or a pharmaceutically acceptable salt thereof,
   wherein W is —OH or —SH.

In some embodiments, the RNAi agent includes the following structure:

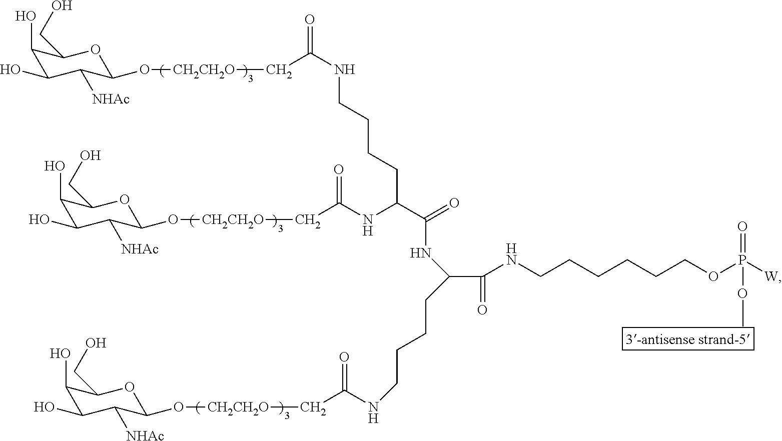

or a pharmaceutically acceptable salt thereof,
   wherein W is —OH or —SH, and the antisense strand includes any antisense strand selected from SEQ ID NOs: 406-810, 1053-1293, 1298-1301, 1441-1447, and 1463-1477.

In some embodiments, W is —OH. In some embodiments, W is —SH.

In certain aspects, the ligand may further include an inverted abasic deoxyribonucleotide (invAb) that may be connected to the sense strand or antisense strand. In some embodiments, the ligand comprises the following structure:

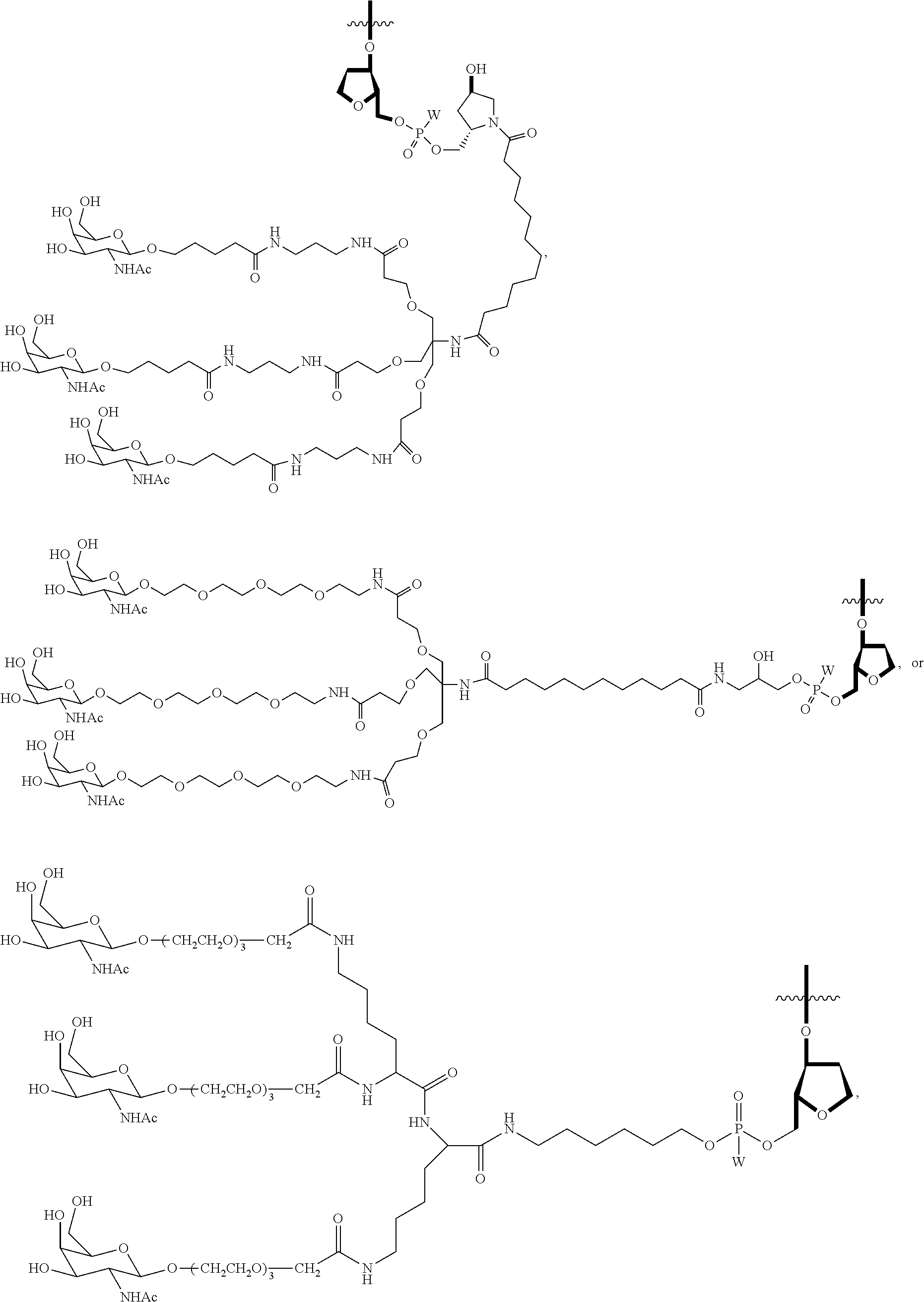

or a pharmaceutically acceptable salt thereof,
   wherein W is —OH or —SH, and [symbol] is an attachment point to the sense strand or the antisense strand.

In some embodiments, W is —OH. In some embodiments, W is —SH.

In certain aspects, the ligand as described above may direct the dsRNAi to a specific cell or tissue, e.g., liver cells. In some embodiments, the ligand may direct the dsRNAi to a liver cell. Examples of dsRNAi agent including the liver targeting ligand (L96) are listed in Table 4.

TABLE 4

| | | | SEQ ID |
|---|---|---|---|
| siRNA | Sequence (5′-3′) | Strand | NO |
| 651 | T005p001G005p001U004pU004pG004pU004pC007pA004pA007pG0 | SS | 1302 |
| | 07pA007pC004pU004pU004pU004pU004pU004pC004pG004pA005p | | |
| | A005px1085 | | |
| | X033U1027p001U007p001C004pG004pA004pA007pA004pA004pA0 | AS | 1306 |
| | 04pG004pU004pC004pU004pU007pG004pA007pC004pA004pA004p | | |
| | C004pA004p001U004p001U004 | | |
| 652 | T005p001T005p001G004pC004pA004pG004pA007pU004pG007pC0 | SS | 1303 |
| | 07pU007pA004pG004pG004pU004pG004pU004pU004pC004pA005p | | |
| | A005px1085 | | |
| | X033U1027p001U007p001G004pA004pA004pC007pA004pC004pC0 | AS | 1307 |
| | 04pU004pA004pG004pC004pA007pU004pC007pU004pG004pC004p | | |
| | A004pA004p001A004p001C004 | | |
| 653 | T005p001C005*p001A004pA004pG004pA004pC007pU004pU007pU | SS | 1304 |
| | 007pU007pU004pC004pG004pA004pA004pU004pG004pC004pA005 | | |
| | pA005px1085 | | |
| | X033U1027p001U007p001G004pC004pA1016pU004pU004pC004pG | AS | 1308 |
| | 004pA004pA004pA004pA004pA007pG004pU007pC004pU004pU004 | | |
| | pG004pA004p001C004p001A004 | | |
| 654 | T005p001T005p001G004pC004pA004pG004pA007pU004pG007pC0 | SS | 1305 |
| | 07pU007pA004pG004pG004pU004pG004pU004pU004pC004pA005p | | |
| | T005px1085 | | |
| | A004p001U007p001G004pA004pA004pC007pA004pC004pC004pU0 | AS | 1309 |
| | 04pA004pG004pC004pA007pU004pC007pU004pG004pC004pA004p | | |
| | A004p001A004p001C004 | | |

X1085: L96

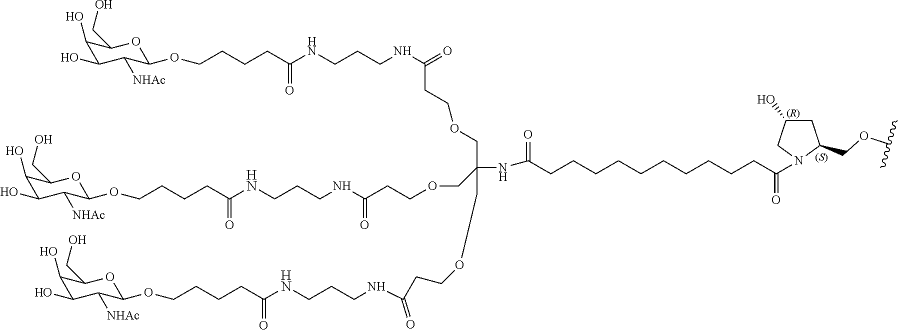

Combinations of dsRNA and ligands as described herein are not limited to the examples and embodiments discussed above.

In some embodiments, a double stranded RNAi agent including:
(i) a sense strand including a nucleotide sequence of SEQ ID NO: 1302; and
(ii) an antisense strand including a nucleotide sequence of SEQ ID NO: 1306,
wherein the ligand (L96) is conjugated to 3′ end of the sense strand to form the following schematic

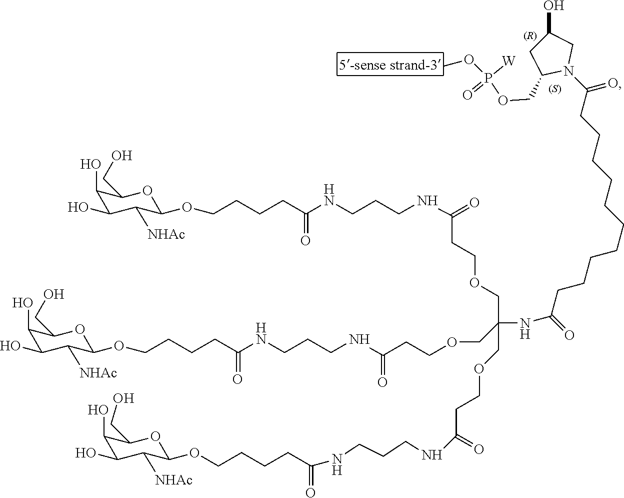

or a pharmaceutically acceptable salt thereof,
   wherein W is —OH or —SH.

In some embodiments, W is —OH.

In some embodiments, a double stranded RNAi agent including:
(i) a sense strand including a nucleotide sequence of SEQ ID NO: 1303; and
(ii) an antisense strand including a nucleotide sequence of SEQ ID NO: 1307,
wherein the ligand (L96) is conjugated to 3′ end of the sense strand to form the following schematic:

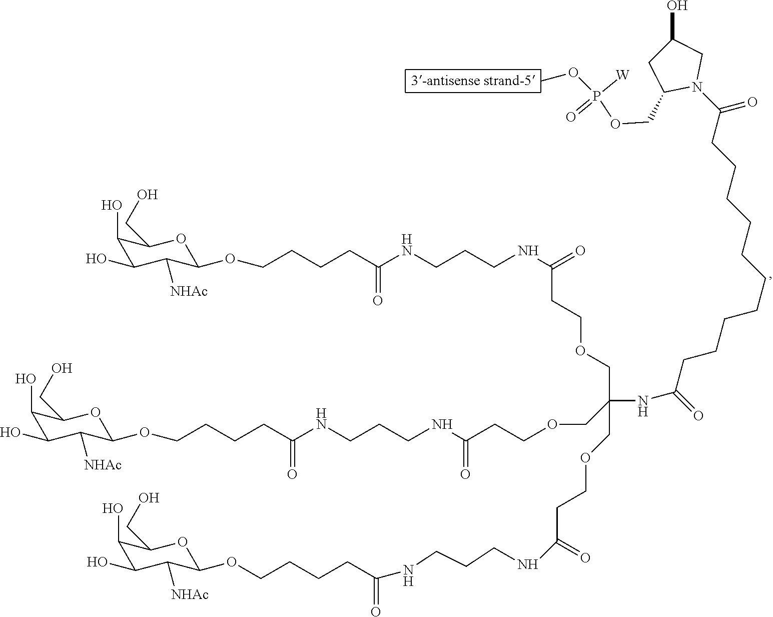

or a pharmaceutically acceptable salt,
   wherein W is —OH or —SH.

In some embodiments, W is —OH.

In some embodiments, a double stranded RNAi agent including:
(i) a sense strand including a nucleotide sequence of SEQ ID NO: 1304; and
(ii) an antisense strand including a nucleotide sequence of SEQ ID NO: 1308,
wherein the ligand (L96) is conjugated to 3′ end of the sense strand to form the following schematic:

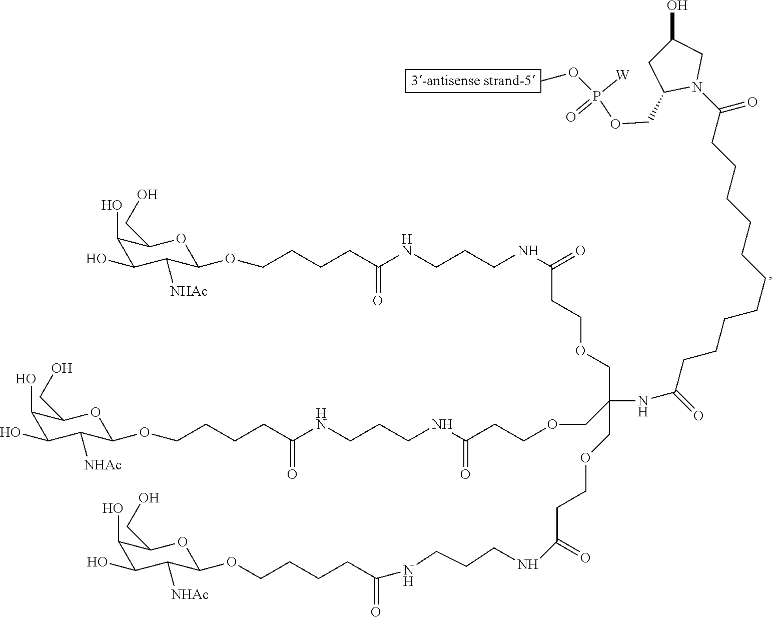

or a pharmaceutically acceptable salt,
   wherein W is —OH or —SH

In some embodiments, W is —OH.

In some embodiments, a double stranded RNAi agent including:
(i) a sense strand including a nucleotide sequence of SEQ ID NO: 1305; and
(ii) an antisense strand including a nucleotide sequence of SEQ ID NO: 1309,
wherein the ligand (L96) is conjugated to 3′ end of the sense strand to form the following schematic:

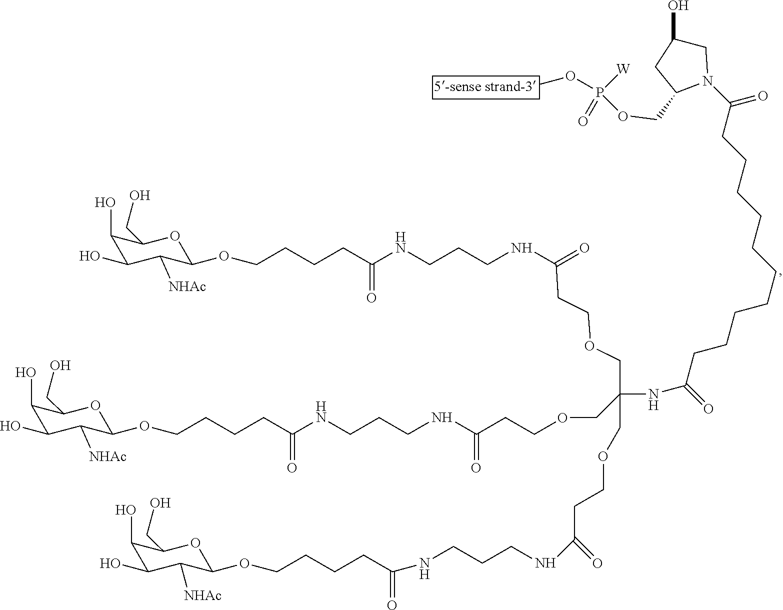

or a pharmaceutically acceptable salt,
wherein W is —OH or —SH.

In some embodiments, W is —OH.

In some embodiments, a double stranded RNAi agent including:
(i) a sense strand including a nucleotide sequence of SEQ ID NO: 1302; and
(ii) an antisense strand including a nucleotide sequence of SEQ ID NO: 1306, wherein the ligand (L96) is conjugated to 3′ end of the sense strand to form the following schematic:

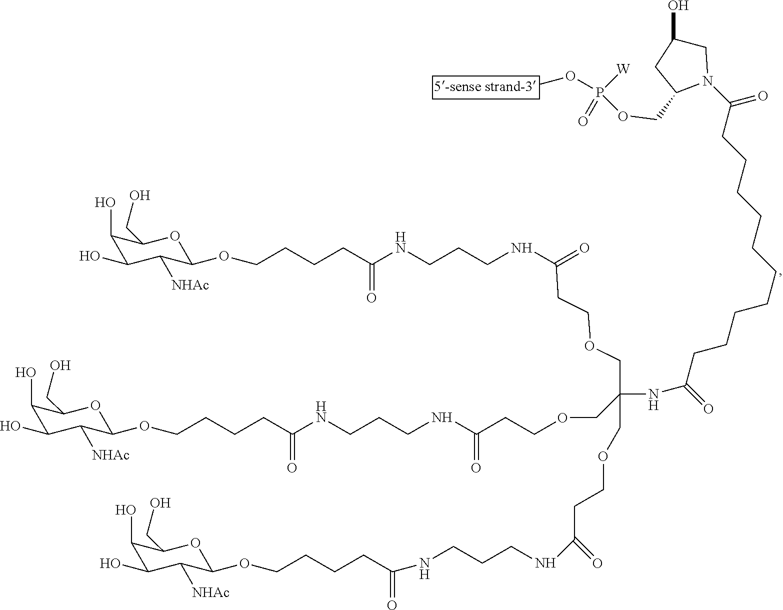

or a pharmaceutically acceptable salt,
   wherein W is —OH.

In some embodiments, a double stranded RNAi agent including:
(i) a sense strand including a nucleotide sequence of SEQ ID NO: 1303; and
(ii) an antisense strand including a nucleotide sequence of SEQ ID NO: 1307,
wherein the ligand (L96) is conjugated to 3′ end of the sense strand to form the following schematic:

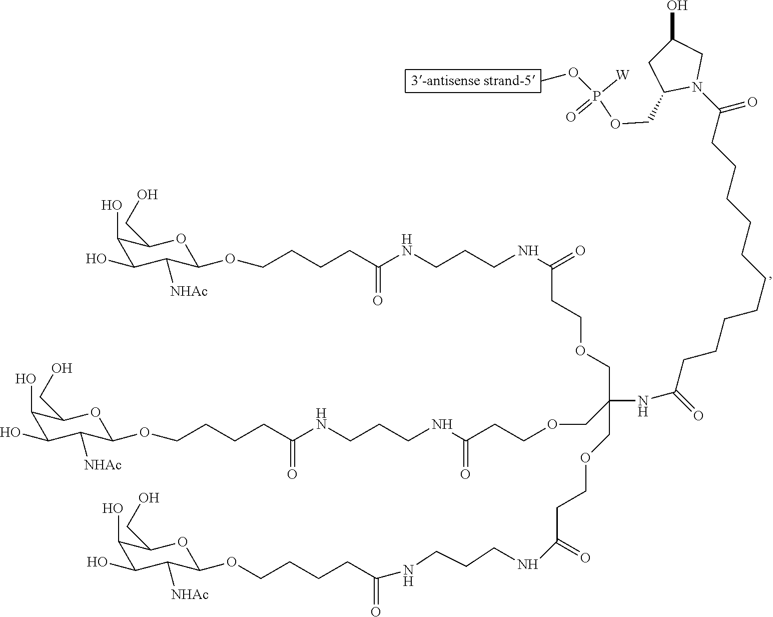

or a pharmaceutically acceptable salt,
wherein W is —OH.

In some embodiments, a double stranded RNAi agent including:
(i) a sense strand including a nucleotide sequence of SEQ ID NO: 1304; and
(ii) an antisense strand including a nucleotide sequence of SEQ ID NO: 1308,
wherein the ligand (L96) is conjugated to 3′ end of the sense strand to form the following schematic:

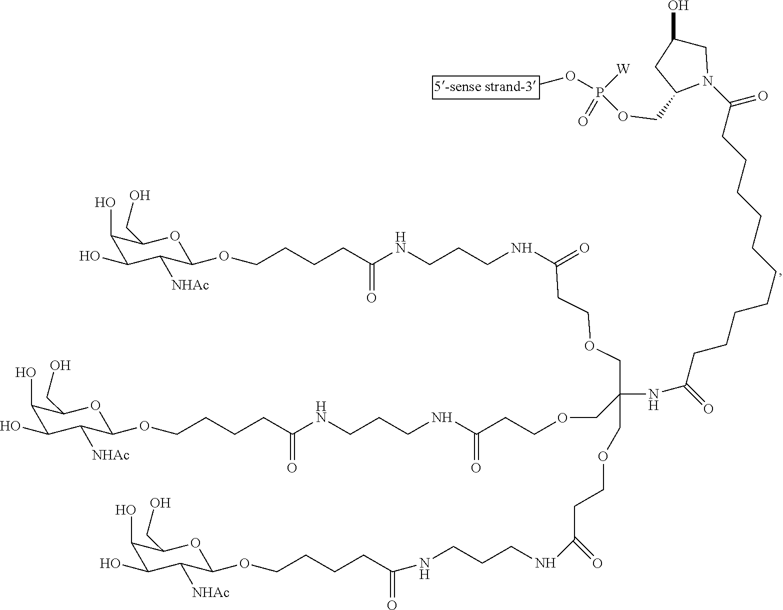

or a pharmaceutically acceptable salt,
wherein W is —OH.

In some embodiments, a double stranded RNAi agent including:
(i) a sense strand including a nucleotide sequence of SEQ ID NO: 1305; and
(ii) an antisense strand including a nucleotide sequence of SEQ ID NO: 1309,
wherein the ligand (L96) is conjugated to 3′ end of the sense strand to form the following schematic:

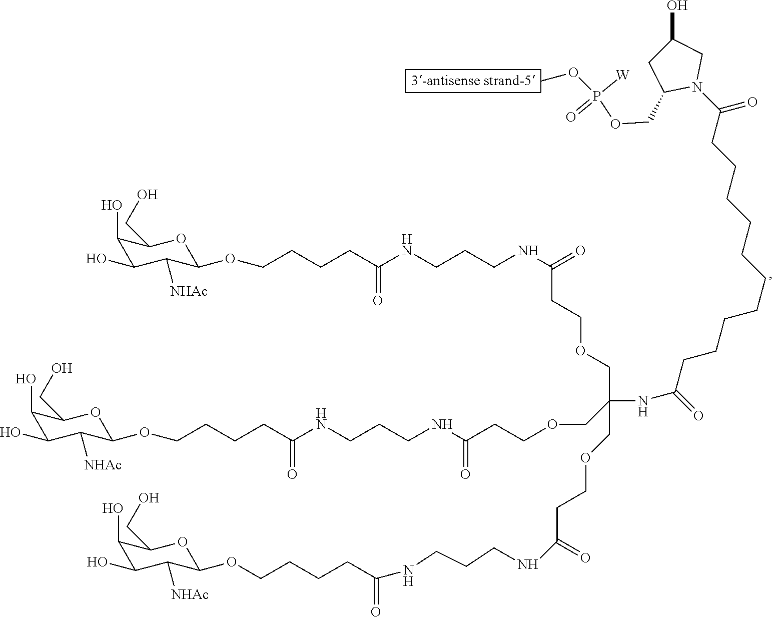

or a pharmaceutically acceptable salt,
wherein W is —OH.

In some embodiments, a double stranded RNAi agent including:
(i) a sense strand including a nucleotide sequence of SEQ ID NO: 1302; and
(ii) an antisense strand including a nucleotide sequence of SEQ ID NO: 1306,
wherein the ligand (L96) is conjugated to 5′ end of the sense strand to form the following schematic:

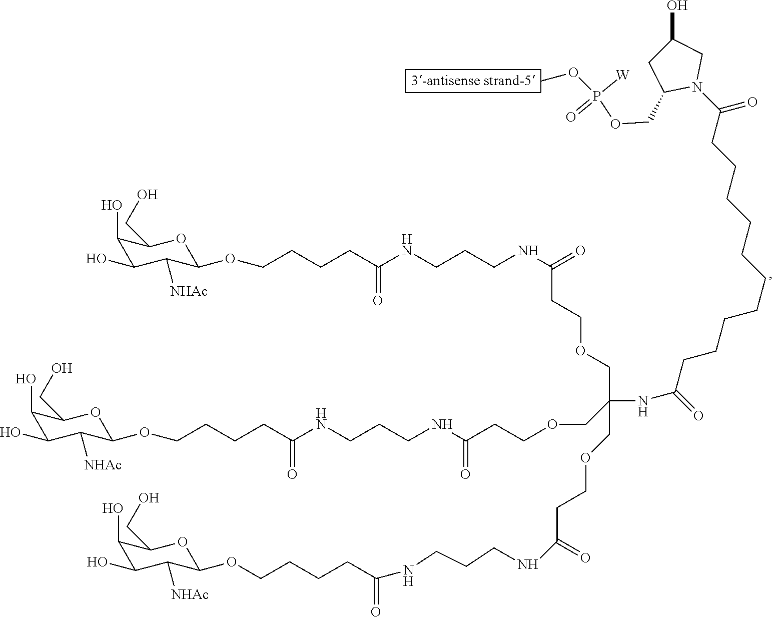

or a pharmaceutically acceptable salt,
wherein W is —OH or —SH.

In some embodiments, W is —OH.

In some embodiments, a double stranded RNAi agent including:
(i) a sense strand including a nucleotide sequence of SEQ ID NO: 1303; and
(ii) an antisense strand including a nucleotide sequence of SEQ ID NO: 1307;
wherein the ligand (L96) is conjugated to 5′ end of the sense strand to form the following schematic:

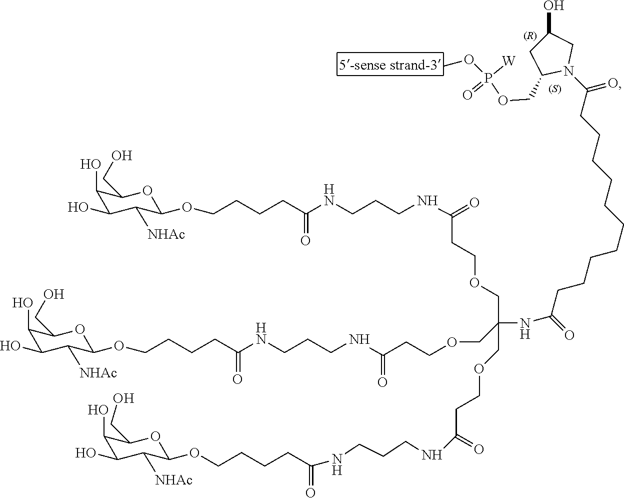

or a pharmaceutically acceptable salt,
wherein W is —OH or —SH.

In some embodiments, W is —OH.

In some embodiments, a double stranded RNAi agent including:
(i) a sense strand including a nucleotide sequence of SEQ ID NO: 1304; and
(ii) an antisense strand including a nucleotide sequence of SEQ ID NO: 1308;
wherein the ligand (L96) is conjugated to 5′ end of the sense strand to form the following schematic:

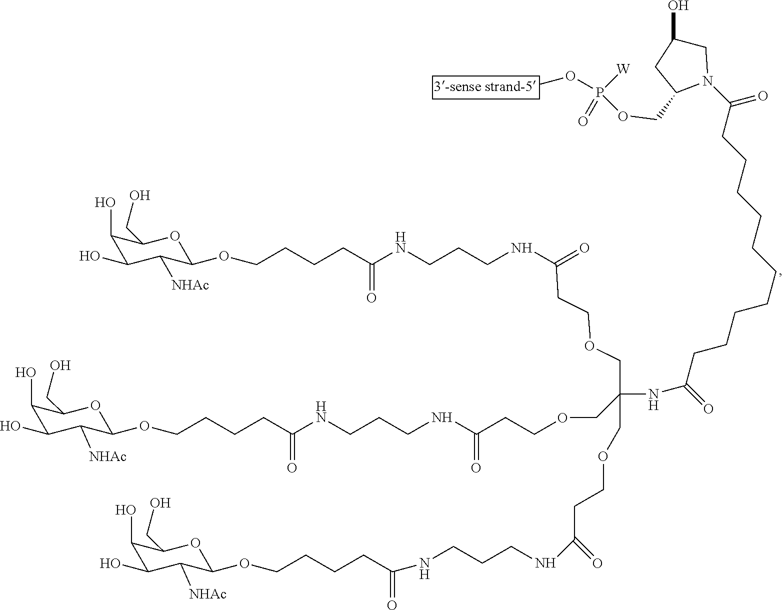

or a pharmaceutically acceptable salt,
wherein W is —OH or —SH.

In some embodiments, W is —OH.

In some embodiments, a double stranded RNAi agent including:
(i) a sense strand including a nucleotide sequence of SEQ ID NO: 1305; and
(ii) an antisense strand including a nucleotide sequence of SEQ ID NO: 1309,
wherein the ligand (L96) is conjugated to 5′ end of the sense strand to form the following schematic:

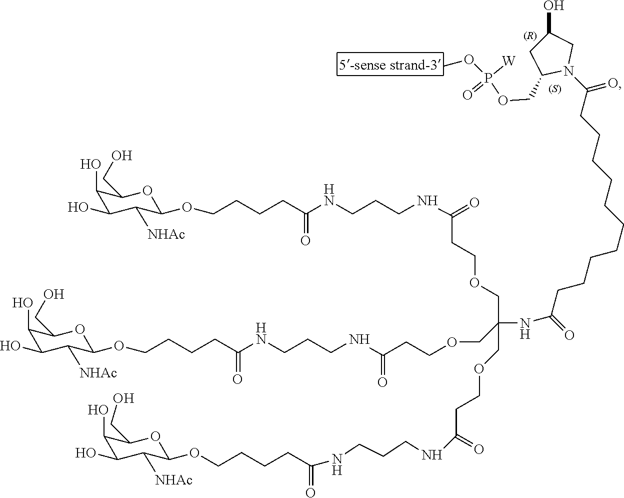

or a pharmaceutically acceptable salt,
wherein W is —OH or —SH.

In some embodiments, W is —OH.

In some embodiments, a double stranded RNAi agent including:
(i) a sense strand including a nucleotide sequence of SEQ ID NO: 1302; and
(ii) an antisense strand including a nucleotide sequence of SEQ ID NO: 1306,
wherein the ligand (L96) is conjugated to 5′ end of the sense strand to form the following schematic:

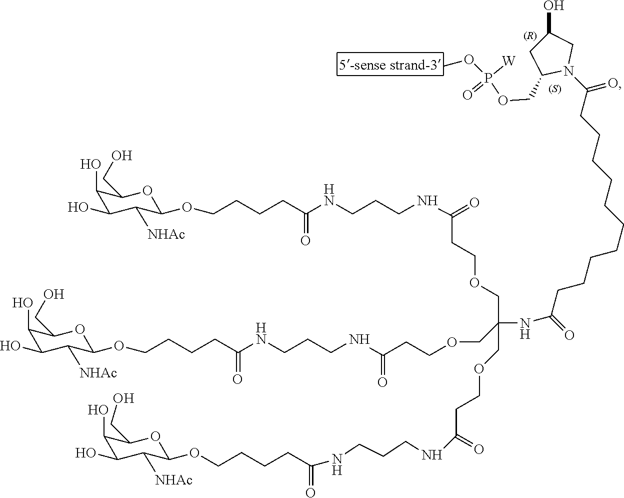

or a pharmaceutically acceptable salt,
wherein W is —OH.

In some embodiments, a double stranded RNAi agent including:
(i) a sense strand including a nucleotide sequence of SEQ ID NO: 1303, and
(ii) an antisense strand including a nucleotide sequence of SEQ ID NO: 1307,
wherein the ligand (L96) is conjugated to 5′ end of the sense strand to form the following schematic:

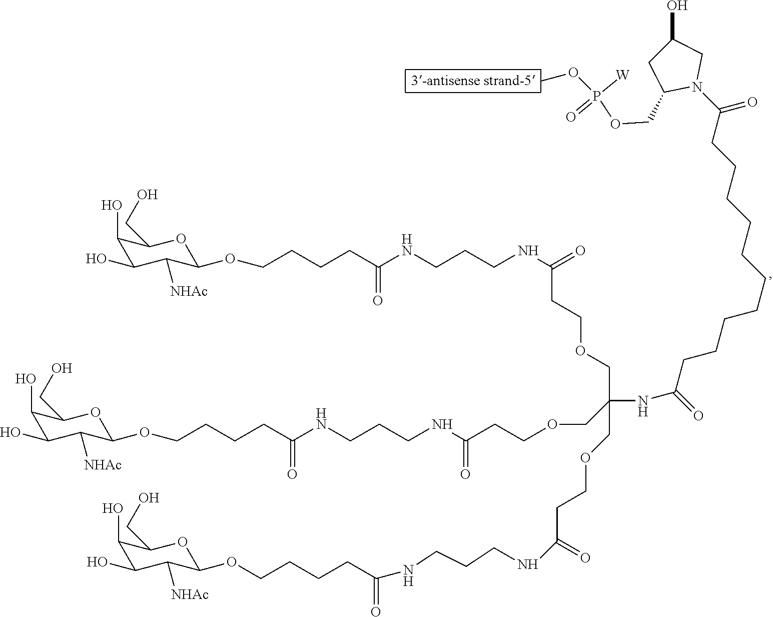

or a pharmaceutically acceptable salt,
wherein W is —OH.

In some embodiments, a double stranded RNAi agent including:
(i) a sense strand including a nucleotide sequence of SEQ ID NO: 1304, and
(ii) an antisense strand including a nucleotide sequence of SEQ ID NO: 1308,
wherein the ligand (L96) is conjugated to 5′ end of the sense strand to form the following schematic:

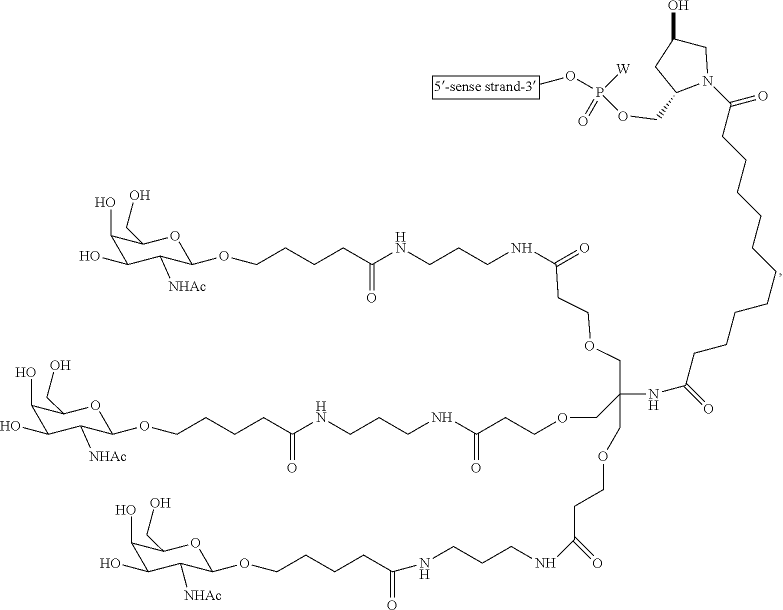

or a pharmaceutically acceptable salt,
wherein W is —OH.

In some embodiments, a double stranded RNAi agent including:
(i) a sense strand including a nucleotide sequence of SEQ ID NO: 1305; and
(ii) an antisense strand including a nucleotide sequence of SEQ ID NO: 1309,
wherein the ligand (L96) is conjugated to 5′ end of the sense strand to form the following schematic:

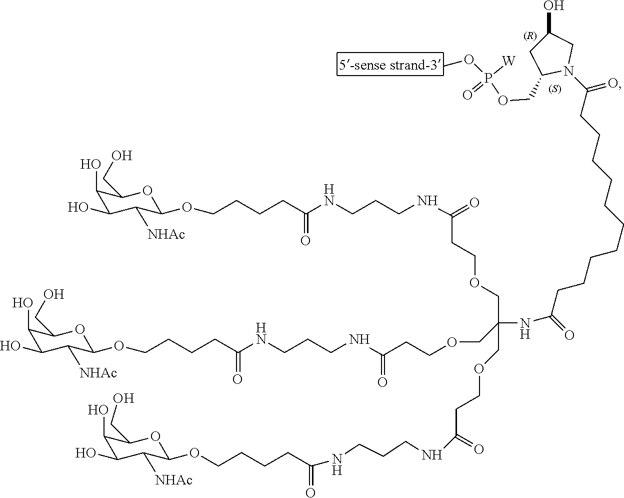

or a pharmaceutically acceptable salt,
wherein W is —OH.

In some embodiments, a double stranded RNAi agent including:
(i) a sense strand consisting of a nucleotide sequence of SEQ ID NO: 1302; and
(ii) an antisense strand consisting of a nucleotide sequence of SEQ ID NO: 1306;
wherein the ligand (L96) is conjugated to 3′ end of the sense strand to form the following schematic:

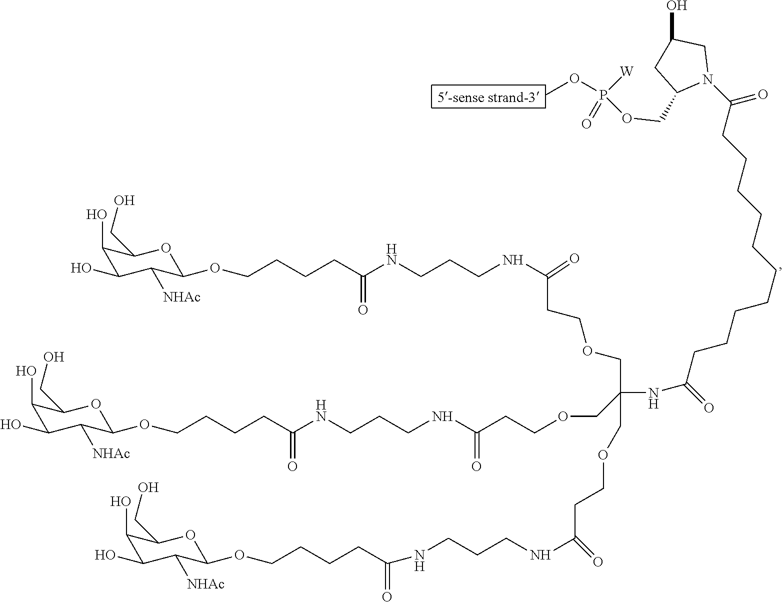

or a pharmaceutically acceptable salt,
   wherein W is —OH or —SH.

In some embodiments, W is —OH.

In some embodiments, a double stranded RNAi agent including:
(i) a sense strand consisting of a nucleotide sequence of SEQ ID NO: 1303; and
(ii) an antisense strand consisting of a nucleotide sequence of SEQ ID NO: 1307;
wherein the ligand (L96) is conjugated to 3′ end of the sense strand to form the following schematic:

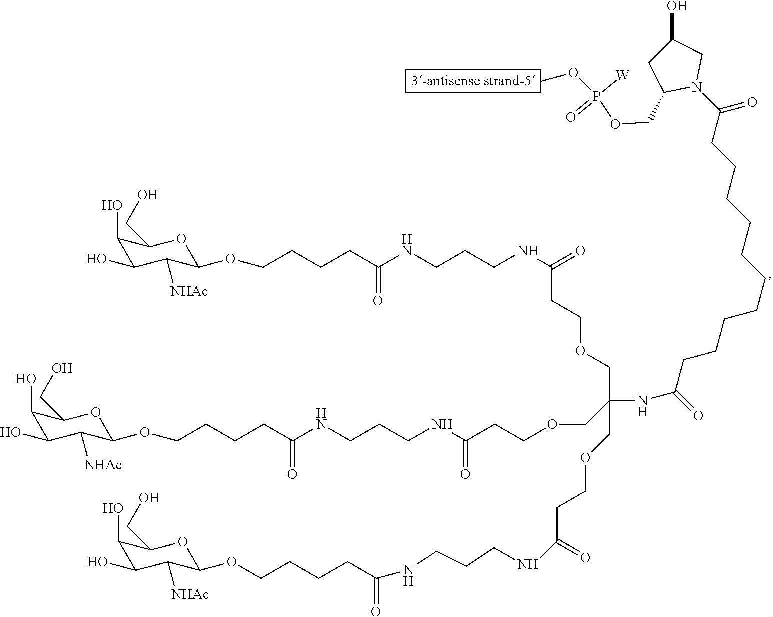

or a pharmaceutically acceptable salt,
wherein W is —OH or —SH.

In some embodiments, W is —OH.

In some embodiments, a double stranded RNAi agent including:
(i) a sense strand consisting of a nucleotide sequence of SEQ ID NO: 1304; and
(ii) an antisense strand consisting of a nucleotide sequence of SEQ ID NO: 1308;
wherein the ligand (L96) is conjugated to 3′ end of the sense strand to form the following schematic:

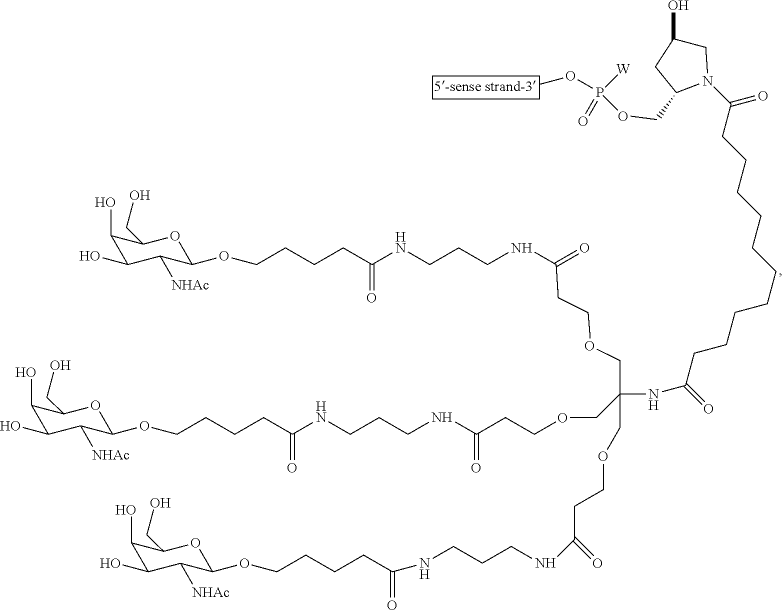

or a pharmaceutically acceptable salt,
wherein W is —OH or —SH.

In some embodiments, W is —OH.

In some embodiments, a double stranded RNAi agent including:
(i) a sense strand consisting of a nucleotide sequence of SEQ ID NO: 1305; and
(ii) an antisense strand consisting of a nucleotide sequence of SEQ ID NO: 1309,
wherein the ligand (L96) is conjugated to 3′ end of the sense strand to form the following schematic:

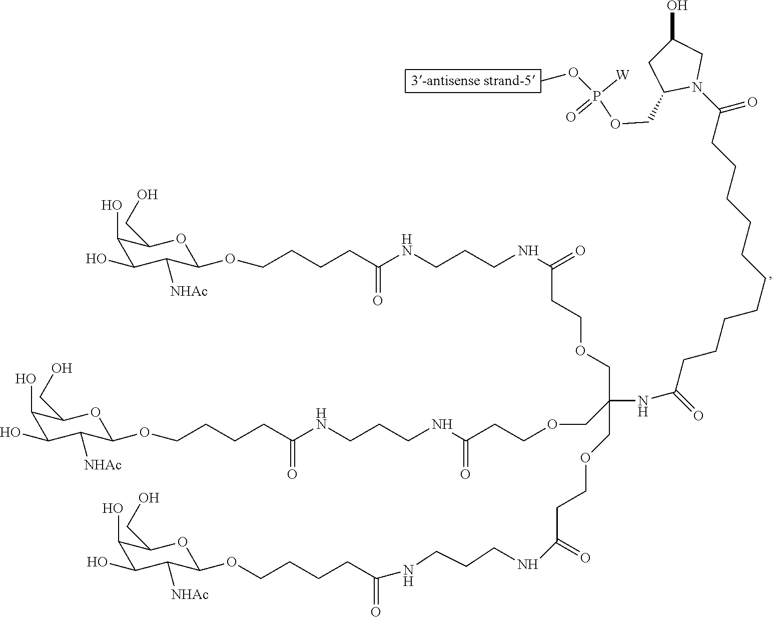

or a pharmaceutically acceptable salt.
wherein W is —OH or —SH.

In some embodiments, W is —OH.

In some embodiments, a double stranded RNAi agent including:
(i) a sense strand consisting of a nucleotide sequence of SEQ ID NO: 1302; and
(ii) an antisense strand consisting of a nucleotide sequence of SEQ ID NO: 1306,
wherein the ligand (L96) is conjugated to 3′ end of the sense strand to form the following schematic:

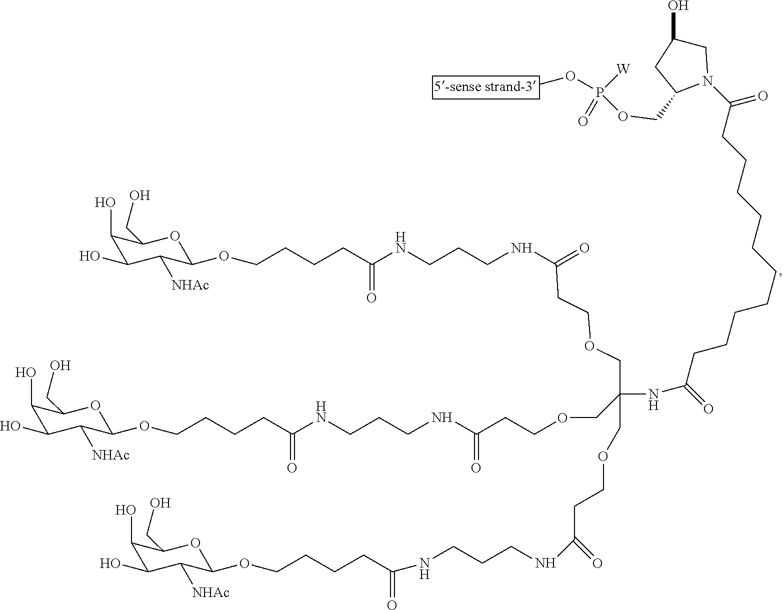

or a pharmaceutically acceptable salt,
wherein W is —OH.

In some embodiments, a double stranded RNAi agent including:
(i) a sense strand consisting of a nucleotide sequence of SEQ ID NO: 1303; and
(ii) an antisense strand consisting of a nucleotide sequence of SEQ ID NO: 1307,
wherein the ligand (L96) is conjugated to 3′ end of the sense strand to form the following schematic:

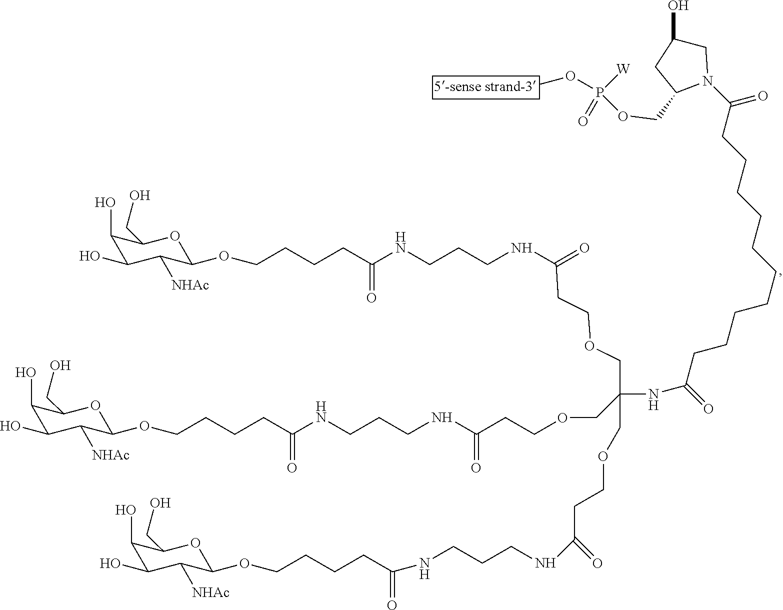

or a pharmaceutically acceptable salt,
wherein W is —OH.

In some embodiments, a double stranded RNAi agent including:
(i) a sense strand consisting of a nucleotide sequence of SEQ ID NO: 1304; and
(ii) an antisense strand consisting of a nucleotide sequence of SEQ ID NO: 1308,
wherein the ligand (L96) is conjugated to 3′ end of the sense strand to form the following schematic:

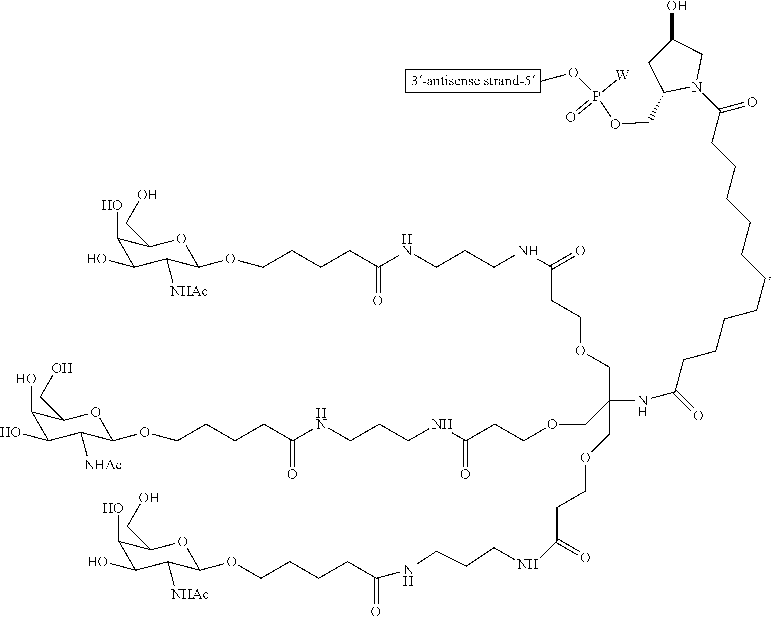

or a pharmaceutically acceptable salt,
wherein W is —OH.

In some embodiments, a double stranded RNAi agent including:
(i) a sense strand consisting of a nucleotide sequence of SEQ ID NO: 1305; and
(ii) an antisense strand consisting of a nucleotide sequence of SEQ ID NO: 1309,
wherein the ligand (L96) is conjugated to 3′ end of the sense strand to form the following schematic:

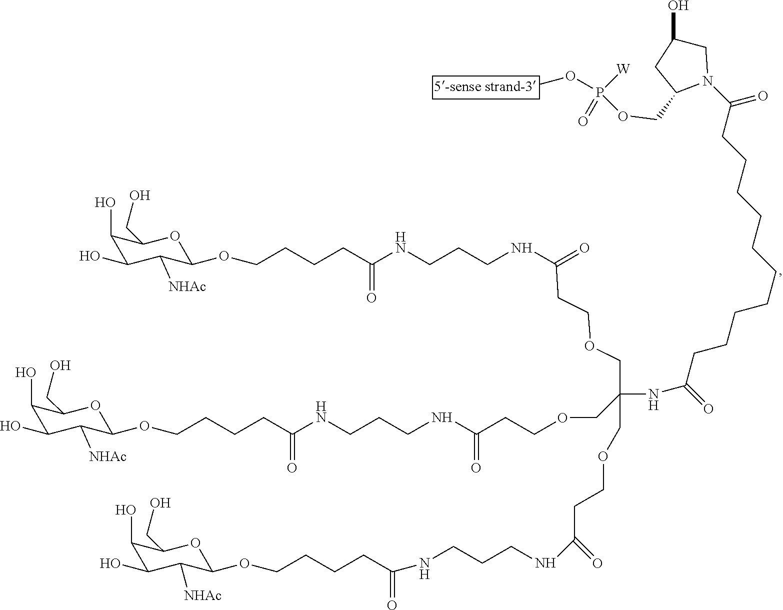

or a pharmaceutically acceptable salt,
wherein W is —OH.

In some embodiments, a double stranded RNAi agent including:
(i) a sense strand consisting of a nucleotide sequence of SEQ ID NO: 1302; and
(ii) an antisense strand consisting of a nucleotide sequence of SEQ ID NO: 1306,
wherein the ligand (L96) is conjugated to 5′ end of the sense strand to form the following schematic:

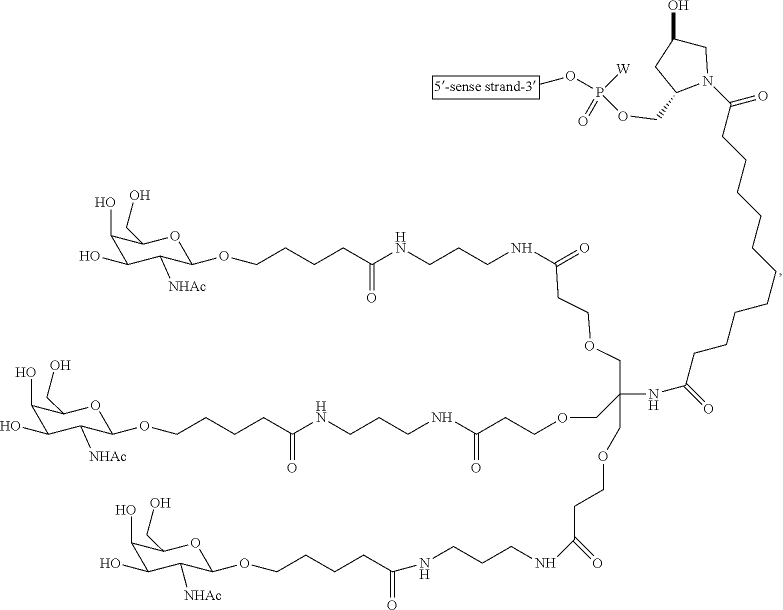

or a pharmaceutically acceptable salt,
wherein W is —OH or —SH.

In some embodiments, W is —OH.

In some embodiments, a double stranded RNAi agent including:
(i) a sense strand consisting of a nucleotide sequence of SEQ ID NO: 1303; and
(ii) an antisense strand consisting of a nucleotide sequence of SEQ ID NO: 1307,
wherein the ligand (L96) is conjugated to 5′ end of the sense strand to form the following schematic:

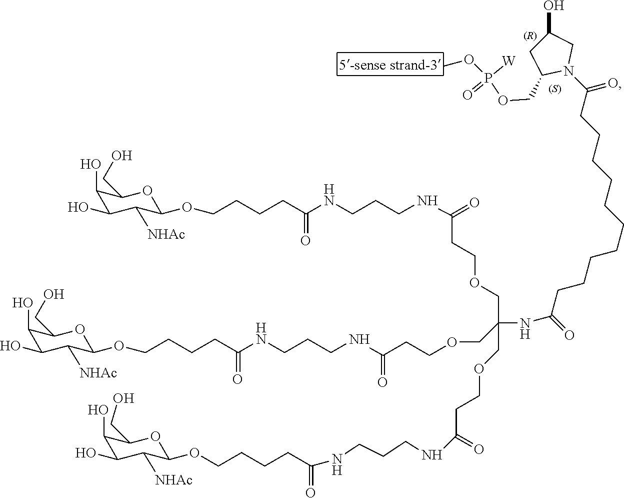

or a pharmaceutically acceptable salt,
wherein W is —OH or —SH.

In some embodiments, W is —OH.

In some embodiments, a double stranded RNAi agent including:
(i) a sense strand consisting of a nucleotide sequence of SEQ ID NO: 1304, and
(ii) an antisense strand consisting of a nucleotide sequence of SEQ ID NO: 1308,
wherein the ligand (L96) is conjugated to 5′ end of the sense strand to form the following schematic:

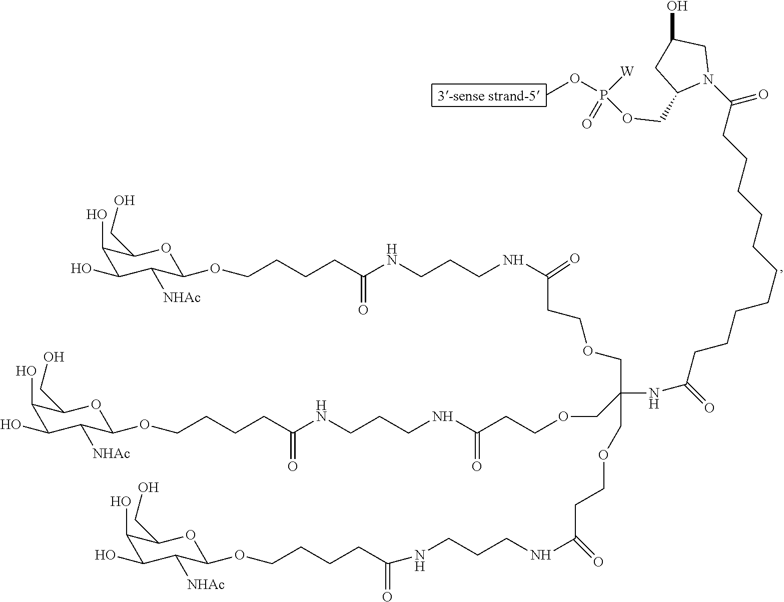

or a pharmaceutically acceptable salt,
wherein W is —OH or —SH.

In some embodiments, W is —OH.

In some embodiments, a double stranded RNAi agent including:
(i) a sense strand consisting of a nucleotide sequence of SEQ ID NO: 1305; and
(ii) an antisense strand consisting of a nucleotide sequence of SEQ ID NO: 1309,
wherein the ligand (L96) is conjugated to 5′ end of the sense strand to form the following schematic:

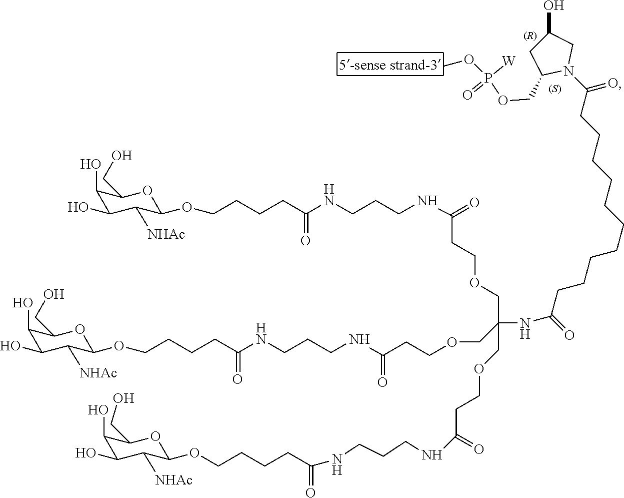

or a pharmaceutically acceptable salt,
wherein W is —OH or —SH.

In some embodiments, W is —OH.

In some embodiments, a double stranded RNAi agent including:
(i) a sense strand consisting of a nucleotide sequence of SEQ ID NO: 1302; and
(ii) an antisense strand consisting of a nucleotide sequence of SEQ ID NO: 1306;
wherein the ligand (L96) is conjugated to 5′ end of the sense strand to form the following schematic:

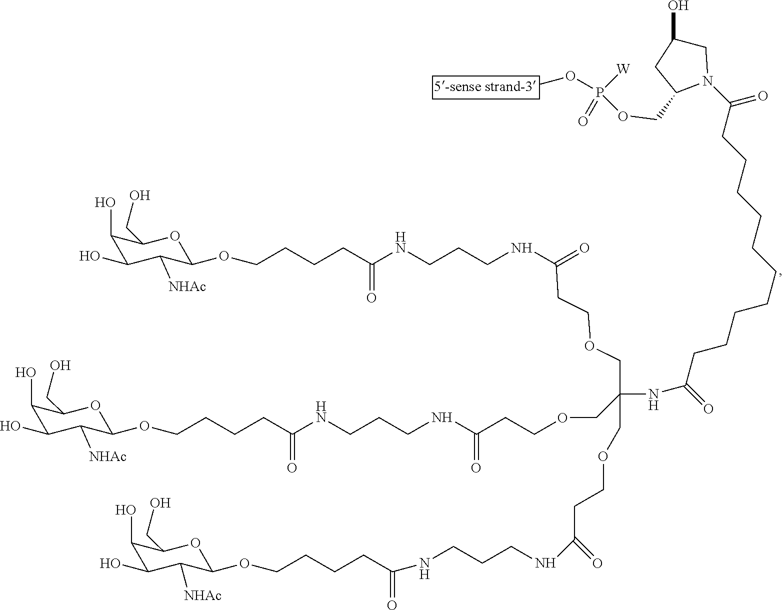

or a pharmaceutically acceptable salt,
wherein W is —OH.

In some embodiments, a double stranded RNAi agent including:
(i) a sense strand consisting of a nucleotide sequence of SEQ ID NO: 1303; and
(ii) an antisense strand consisting of a nucleotide sequence of SEQ ID NO: 1307,
wherein the ligand (L96) is conjugated to 5′ end of the sense strand to form the following schematic:

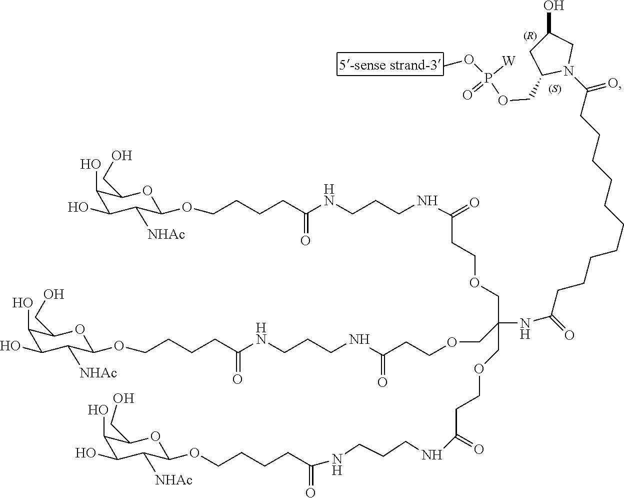

or a pharmaceutically acceptable salt,
wherein W is —OH.

In some embodiments, a double stranded RNAi agent including:
(i) a sense strand consisting of a nucleotide sequence of SEQ ID NO: 1304; and
(ii) an antisense strand consisting of a nucleotide sequence of SEQ ID NO: 1308,
wherein the ligand (L96) is conjugated to 5′ end of the sense strand to form the following schematic:

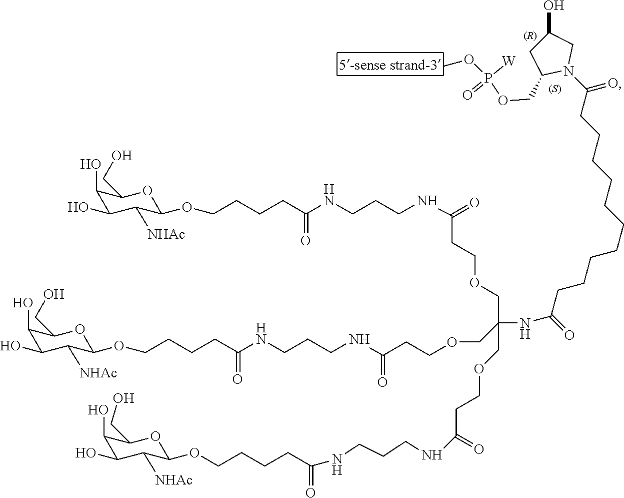

or a pharmaceutically acceptable salt,
wherein W is —OH.

In some embodiments, a double stranded RNAi agent including:
(i) a sense strand consisting of a nucleotide sequence of SEQ ID NO: 1305; and
(ii) an antisense strand consisting of a nucleotide sequence of SEQ ID NO: 1309,
wherein the ligand (L96) is conjugated to 5′ end of the sense strand to form the following schematic:

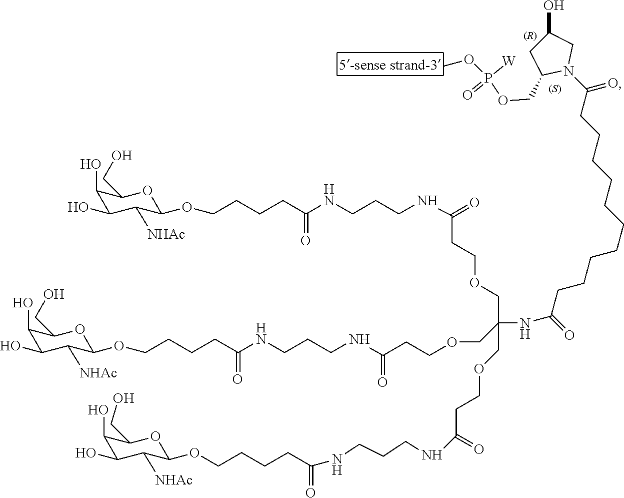

or a pharmaceutically acceptable salt,
wherein W is —OH.

In some embodiments, a double stranded RNAi agent (e.g., siRNA agent) includes:

(i) a sense strand (SS) comprising a nucleotide sequence of
(SEQ ID NO: 1294)
5′-
T005p001G005p001U004pU004pG004pU004pC007pA004pA007pG007pA007pC004pU004
pU004pU004pU004pU004pC004pG004pA005pA005-3′;
(ii) an antisense strand (AS) comprising a nucleotide sequence of
(SEQ ID NO: 1298)
5′-
X033U1027p001U007p001C004pG004pA004pA007pA004pA004pA004pG004pU004pC004
pu004pU007pG004pA007pC004pA004pA004pC004pA004p001U004p001U004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
wherein the ligand (L96) is

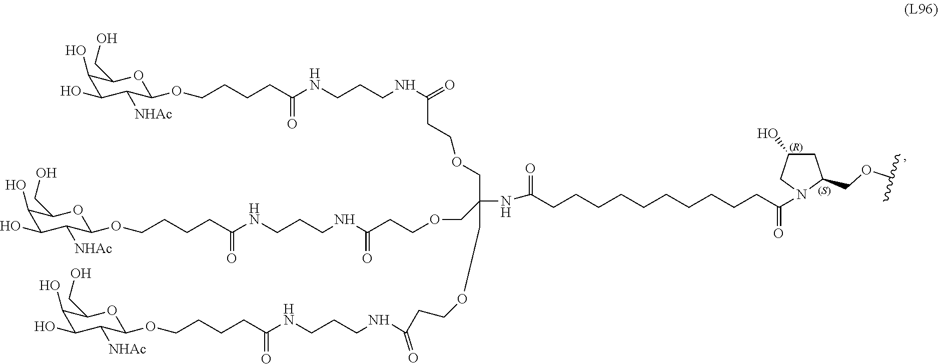

wherein the ligand is conjugated to 3′ end of the sense strand (SS), e.g., via a phosphodiester or phosphorothioate linkage to form the following schematic:

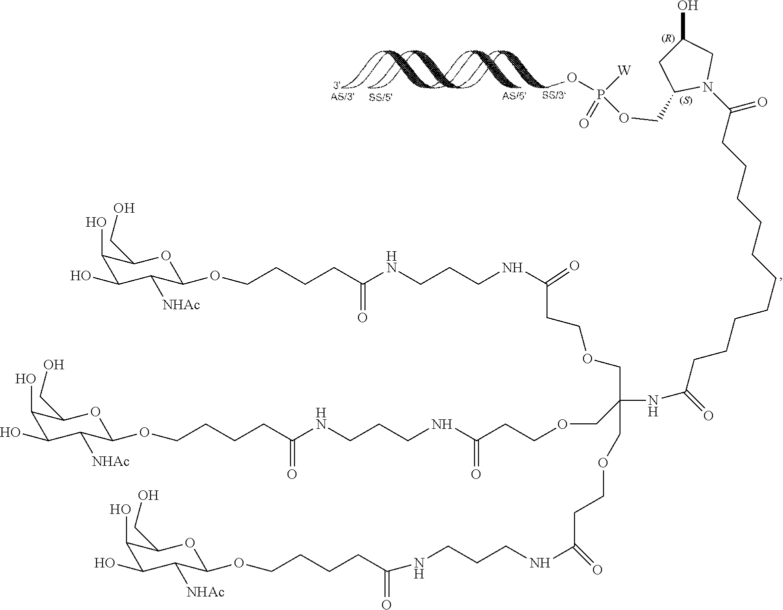

wherein W is —OH,
or a pharmaceutically acceptable salt thereof (e.g., sodium salt form).

In some embodiments, a double stranded RNAi agent (e.g., siRNA agent) includes:

(i) a sense strand (SS) consisting of a nucleotide sequence of:
(SEQ ID NO: 1294)
5′-
T005p001G005p001U004pU004pG004pU004pC007pA004pA007pG007pA007pC004pU004
pU004pU004pU004pU004pC004pG004pA005pA005-3′;
(ii) an antisense strand (AS) consisting of a nucleotide sequence of:
(SEQ ID NO: 1298)
5′-
X033U1027p001U007p001C004pG004pA004pA007pA004pA004pA004pG004pU004pC004
pU004pU007pG004pA007pC004pA004pA004pC004pA004p001U004p001U004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
wherein the ligand (L96) is

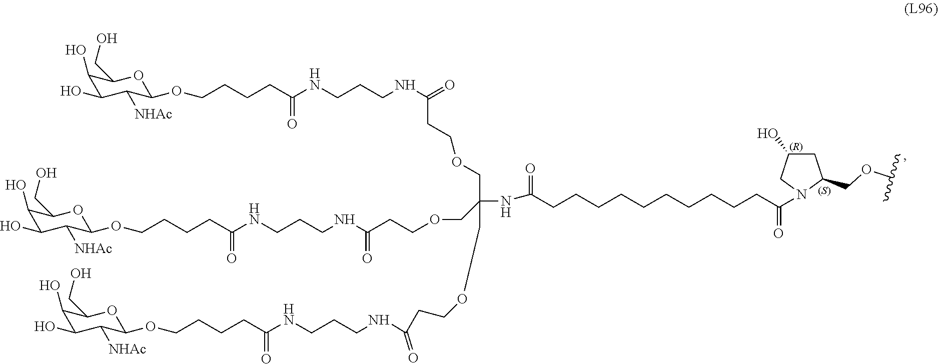

wherein the ligand is conjugated to 3′ end of the sense strand (SS), e.g., via a phosphodiester or phosphorothioate linkage, to form the following schematic:

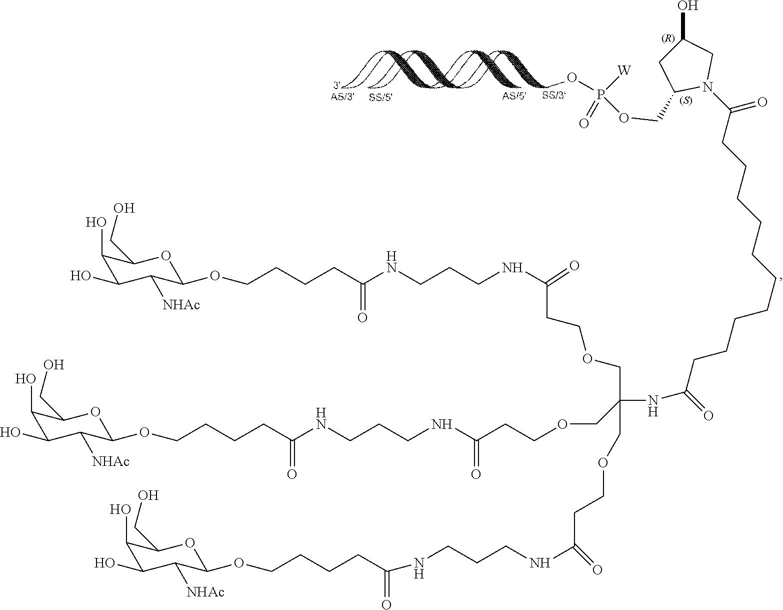

wherein W is —OH,
or a pharmaceutically acceptable salt thereof (e.g., sodium salt form).

In some embodiments, a double stranded RNAi agent (e.g., siRNA agent) includes:

(i)
a sense strand (SS) comprising a nucleotide sequence of
(SEQ ID NO: 1294)
5′-
T005p001G005p001U004pU004pG004pU004pC007pA004pA007pG007pA007pC004pU004
pU004pU004pU004pU004pC004pG004pA005pA005-3′;
(ii) an antisense strand (AS) comprising a nucleotide sequence of
(SEQ ID NO: 1298)
5′-
X033U1027p001U007p001C004pG004pA004pA007pA004pA004pA004pG004pU004pC004
pU004pU007pG004pA007pC004pA004pA004pC004pA004p001U004p001U004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl(MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
wherein the ligand (L96) is

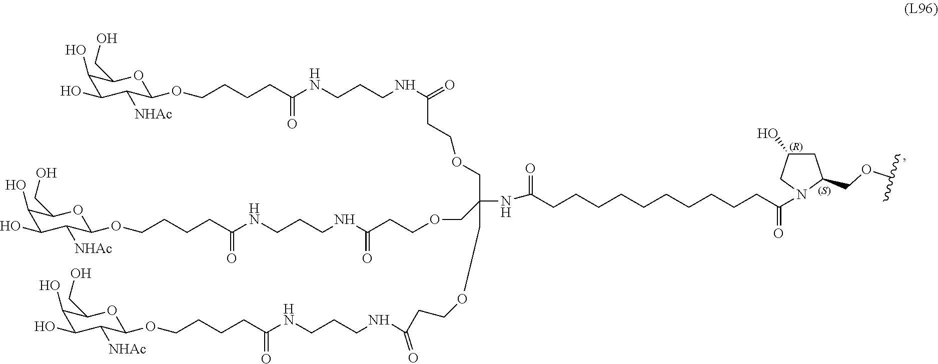

wherein the ligand is conjugated to 3′ end of the sense strand (SS) via a phosphodiester linkage to form the following schematic:

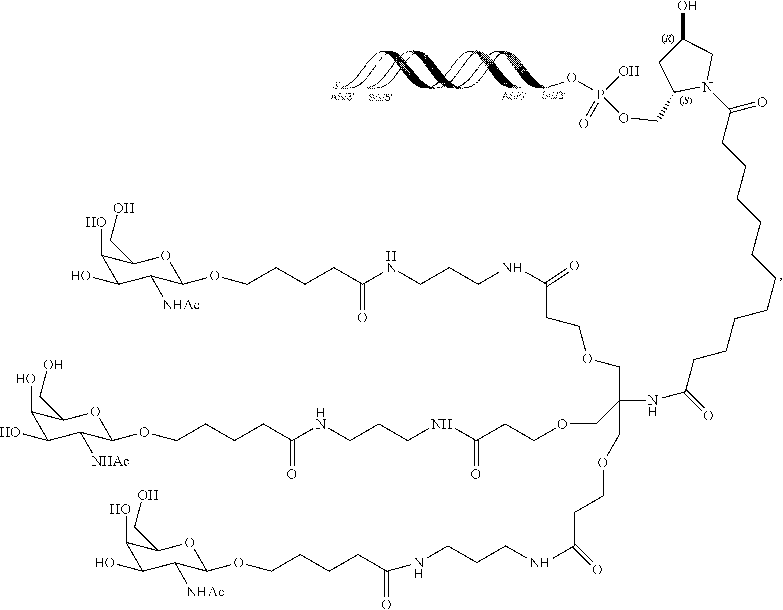

or a pharmaceutically acceptable salt thereof (e.g., sodium salt form).

In some embodiments, a double stranded RNAi agent (e.g., siRNA agent) includes:

(i) a sense strand (SS) consisting of a nucleotide sequence of:
(SEQ ID NO: 1294)
5′-
T005p001G005p001U004pU004pG004pU004pC007pA004pA007pG007pA007pC004pU004
pU004pU004pU004pU004pC004pG004pA005pA005-3′;
(ii) an antisense strand (AS) consisting of a nucleotide sequence of:
(SEQ ID NO: 1298)
5′-
X033U1027p001U007p001C004pG004pA004pA007pA004pA004pA004pG004pU004pC004
pU004pU007pG004pA007pC004pA004pA004pC004pA004p001U004p001U004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
wherein the ligand (L96) is

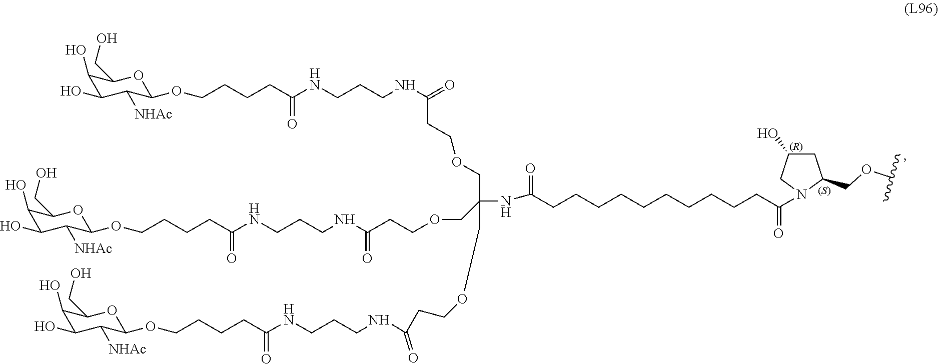

wherein the ligand is conjugated to 3′ end of the sense strand (SS) via a phosphodiester linkage to form the following schematic:

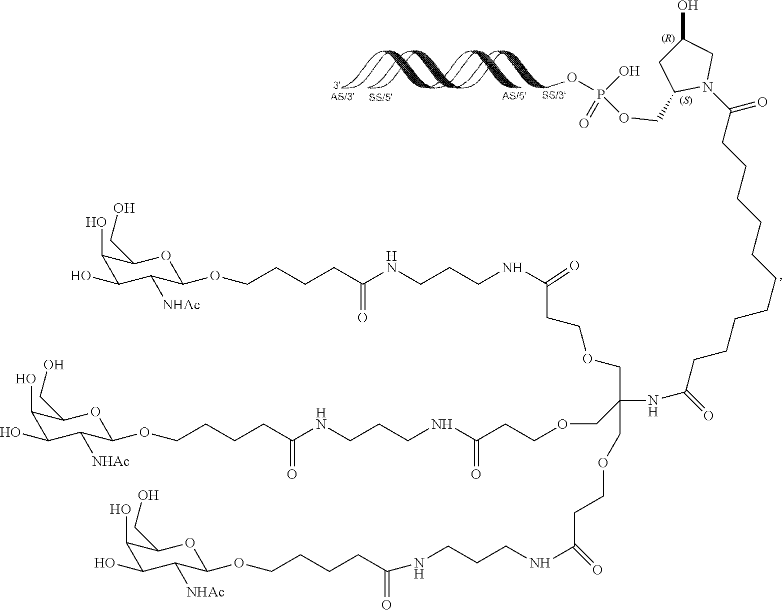

or a pharmaceutically acceptable salt thereof (e.g., sodium salt form).

In some embodiments, a double stranded RNAi agent includes:

(i) a sense strand (SS) comprising a nucleotide sequence of
(SEQ ID NO: 1302)
5′-
T005p001G005p001U004pU004pG004pU004pC007pA004pA007pG007pA007pC004pU004
pU004pU004pU004pU004pC004pG004pA005pA005px1085-3′;
and
(ii) an antisense strand (AS) comprising a nucleotide sequence of
(SEQ ID NO: 1306)
5′-
X033U1027p001U007p001C004pG004pA004pA007pA004pA004pA004pG004pU004pC004
pU004pU007pG004pA007pC004pA004pA004pC004pA004p001U004p001U004-3′;

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; p001 is a phosphorothioate linkage; and X1085 is a ligand (L96),
wherein the ligand (L96) is

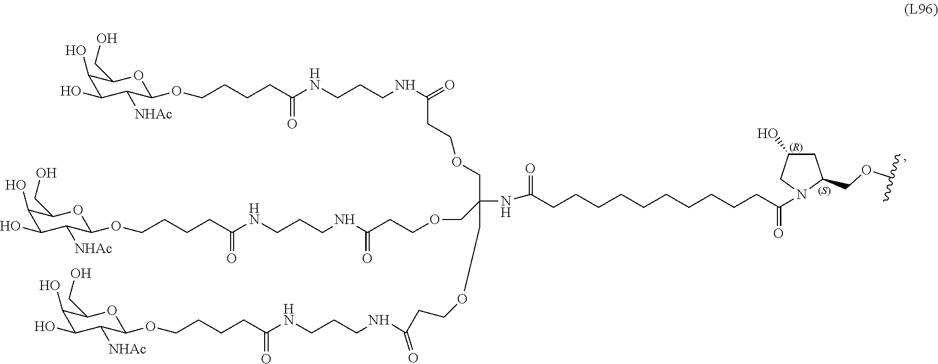

wherein the dsRNAi has the following schematic:

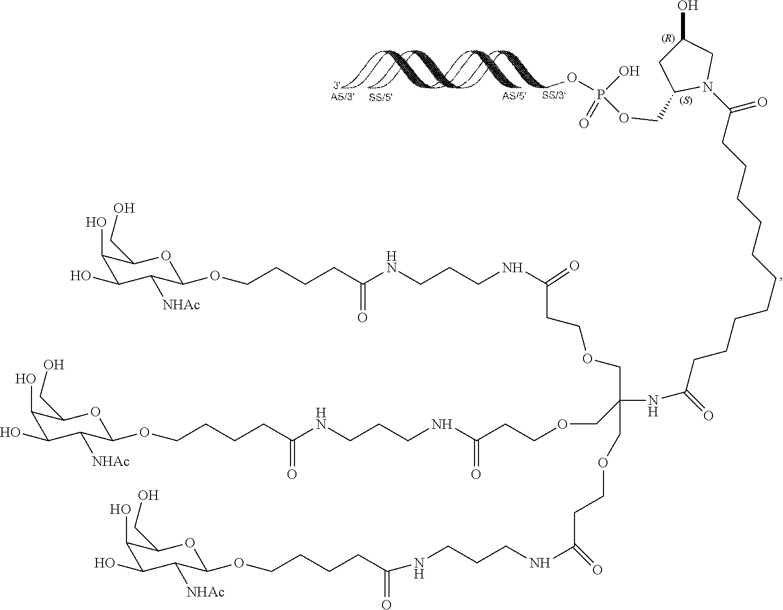

or a pharmaceutically acceptable salt thereof (e.g., sodium salt form).

In some embodiments, a double stranded RNAi agent includes:

(i) a sense strand (SS) consisting of a nucleotide sequence of
(SEQ ID NO: 1302)
5′-
T005p001G005p001U004pU004pG004pU004pC007pA004pA007pG007pA007pC004pU004
pU004pU004pU004pU004pC004pG004pA005pA005px1085-3′;
and
(ii) an antisense strand (AS) consisting of a nucleotide sequence of
(SEQ ID NO: 1306)
5′-
X033U1027p001U007p001C004pG004pA004pA007pA004pA004pA004pG004pU004pC004
pU004pU007pG004pA007pC004pA004pA004pC004pA004p001U004p001U004-3′;

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; p001 is a phosphorothioate linkage; and X1085 is a ligand (L96),
wherein the ligand (L96) is

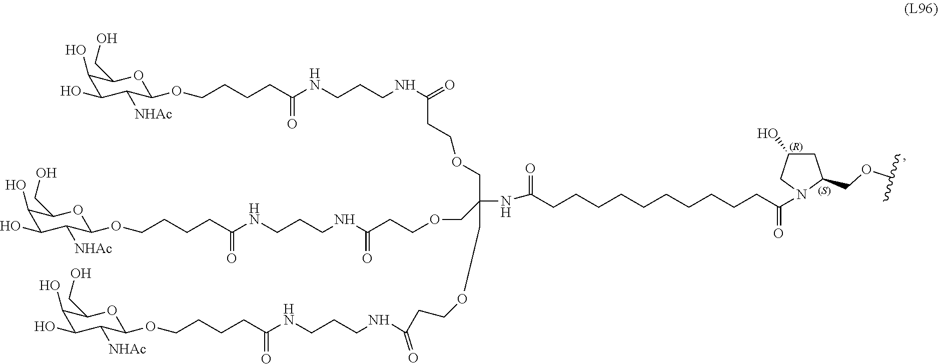

wherein the dsRNAi has the following schematic:

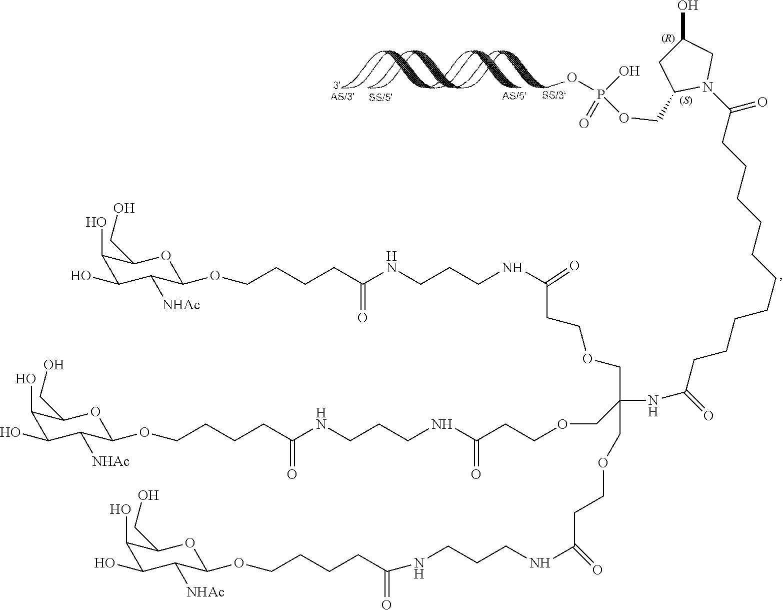

or a pharmaceutically acceptable salt thereof (e.g., sodium salt form).

In some embodiments, a double stranded RNAi agent (e.g., siRNA agent) includes:

(i) a sense strand (SS) comprising
a nucleotide sequence of
(SEQ ID NO: 1295)
5′-T005p001T005p001G004pC004pA004pG004pA007pU
004pG007pC007pU007pA004pG004pG004pU004pG004pU
004pU004pC004pA005pA005-3′;
(ii) an antisense strand (AS) comprising
a nucleotide sequence of
(SEQ ID NO: 1299)
5′-X033U1027p001U007p001G004pA004pA004pC007pA
004pC004pC004pU004pA004pG004pC004pA007pU004pC
007pU004pG004pC004pA004pA004p001A004p001C004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
wherein the ligand (L96) is

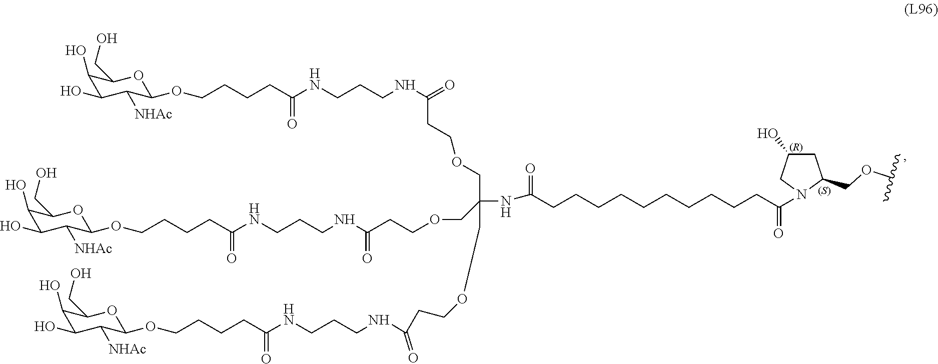

wherein the ligand is conjugated to 3′ end of the sense strand (SS), e.g., via a phosphodiester or phosphorothioate linkage, to form the following schematic:

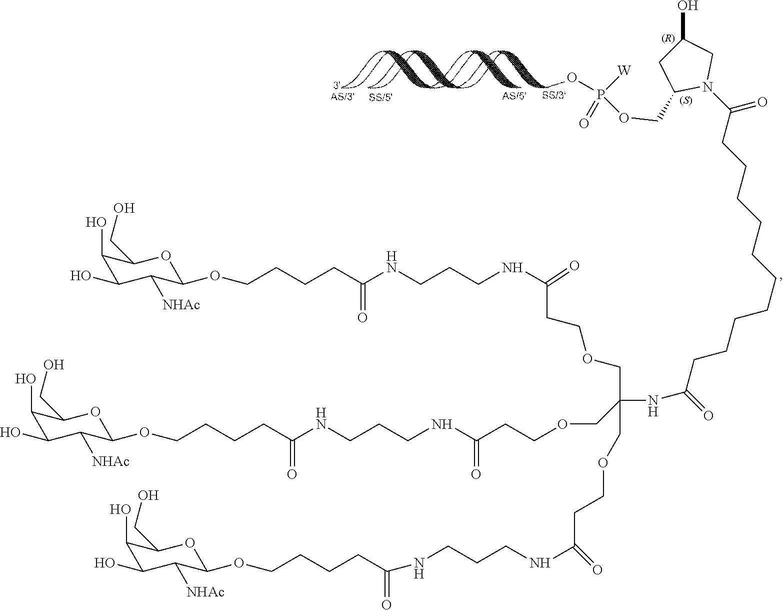

wherein W is —OH,
or a pharmaceutically acceptable salt thereof.

In some embodiments, a double stranded RNAi agent (e.g., siRNA agent) includes:

(i) a sense strand (SS) consisting of
a nucleotide sequence of
(SEQ ID NO: 1295)
5′-T005p001T005p001G004pC004pA004pG004pA007pU
004pG007pC007pU007pA004pG004pG004pU004pG004pU
004pU004pC004pA005pA005-3′;
(ii) an antisense strand (AS) consisting of
a nucleotide sequence of
(SEQ ID NO: 1299)
5′-X033U1027p001U007p001G004pA004pA004pC007pA
004pC004pC004pU004pA004pG004pC004pA007pU004pC
007pU004pG004pC004pA004pA004p001A004p001C004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
wherein the ligand (L96) is

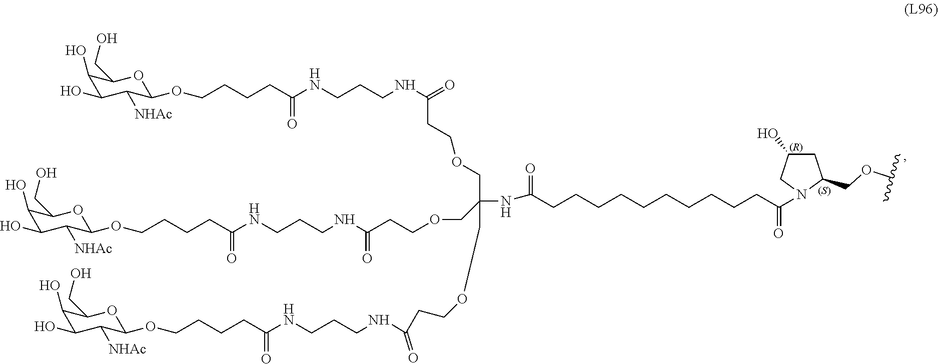

wherein the ligand is conjugated to 3′ end of the sense strand (SS), e.g., via a phosphodiester or phosphorothioate linkage, to form the following schematic:

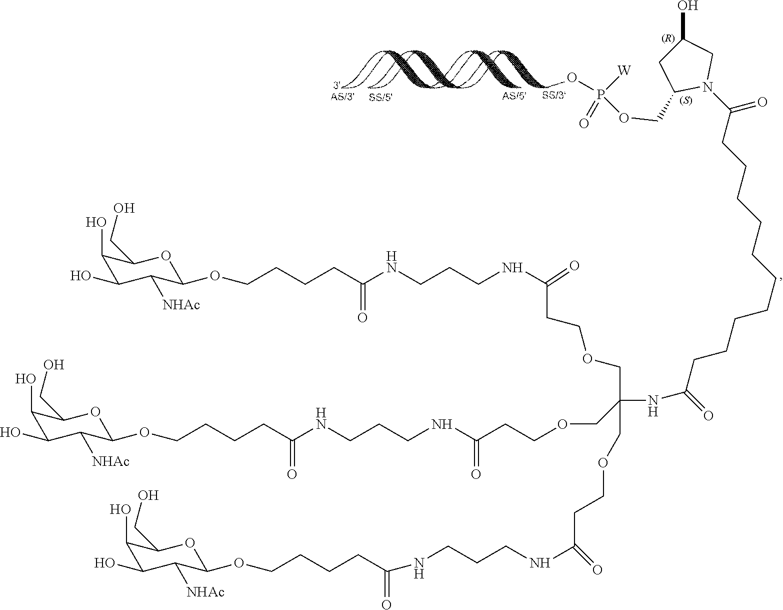

wherein W is —OH,
or a pharmaceutically acceptable salt thereof.

In some embodiments, a double stranded RNAi agent (e.g., siRNA agent) includes:

(i) a sense strand (SS) comprising
a nucleotide sequence of
(SEQ ID NO: 1295)
5′-T005p001T005p001G004pC004pA004pG004pA007pU004
pG007pC007pU007pA004pG004pG004pU004pG004pU004
pU004pC004pA005pA005-3′;
(ii) an antisense strand (AS) comprising
a nucleotide sequence of
(SEQ ID NO: 1299)
5′-X033U1027p001U007p001G004pA004pA004pC007pA
004pC004pC004pU004pA004pG004pC004pA007pU004pC
007pU004pG004pC004pA004pA004p001A004p001C004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
wherein the ligand (L96) is

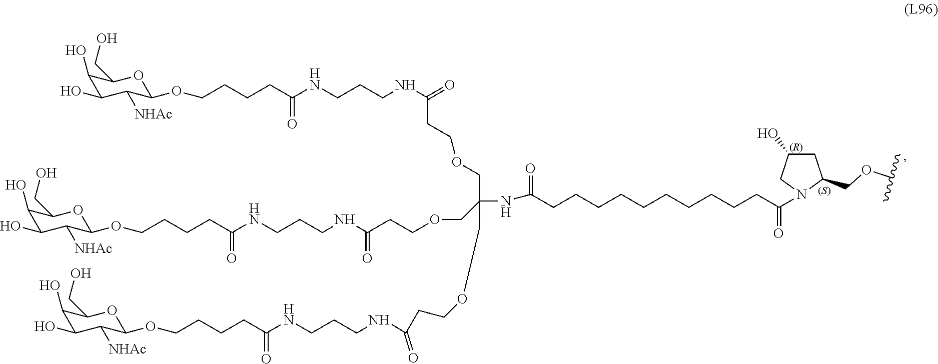

wherein the ligand is conjugated to 3′ end of the sense strand (SS) via a phosphodiester linkage to form the following schematic.

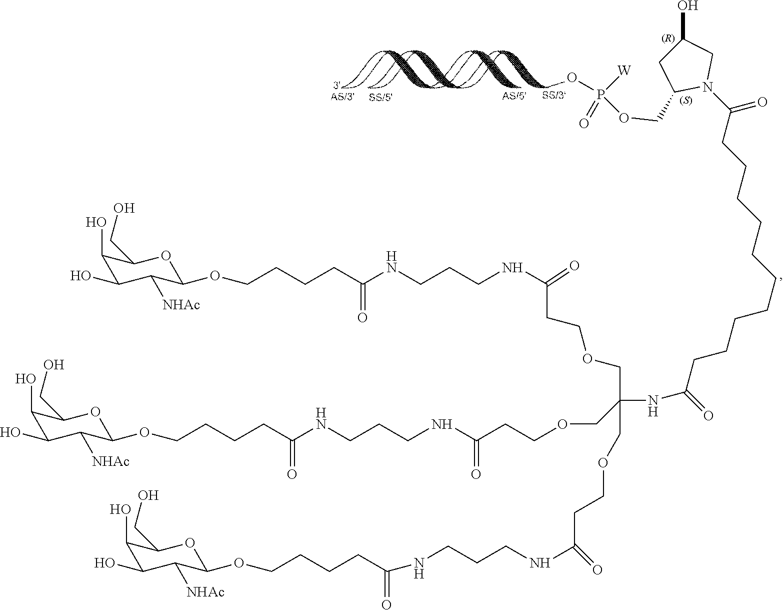

or a pharmaceutically acceptable salt thereof (e.g., sodium salt form).

In some embodiments, a double stranded RNAi agent (e.g., siRNA agent) includes:

(i) a sense strand (SS) consisting of
a nucleotide sequence of
(SEQ ID NO: 1295)
5′-T005p001T005p001G004pC004pA004pG004pA007pU
004pG007pC007pU007pA004pG004pG004pU004pG004pU
004pU004pC004pA005pA005-3′;
(ii) an antisense strand (AS) consisting of
a nucleotide sequence of
(SEQ ID NO: 1299)
5′-X033U1027p001U007p001G004pA004pA004pC007pA
004pC004pC004pU004pA004pG004pC004pA007pU004pC
007pU004pG004pC004pA004pA004p001A004p001C004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
wherein the ligand (L96) is

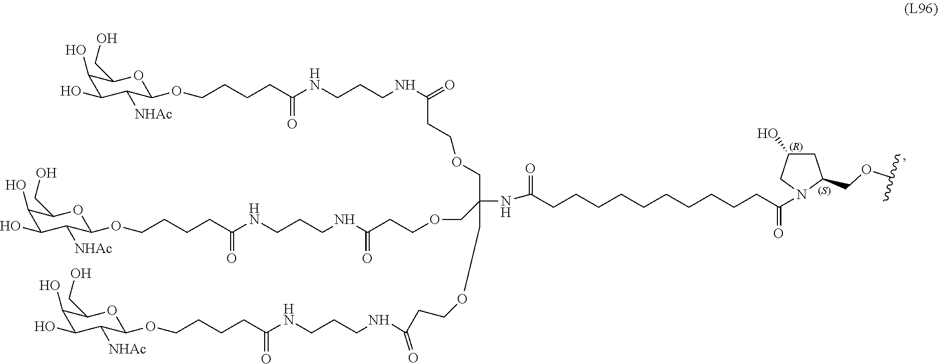

wherein the ligand is conjugated to 3′ end of the sense strand (SS) via a phosphodiester linkage to form the following schematic:

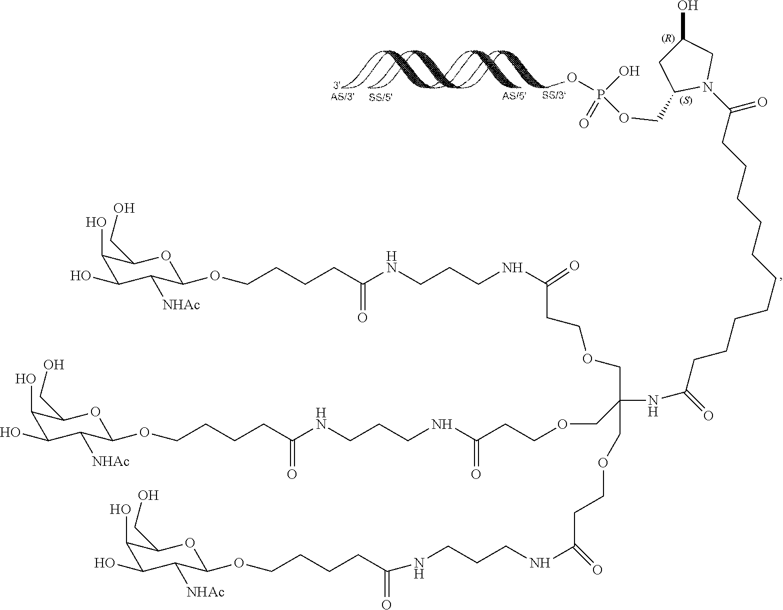

or a pharmaceutically acceptable salt thereof (e.g., sodium salt form).

In some embodiments, a double stranded RNAi agent includes:

(i) a sense strand (SS) comprising
a nucleotide sequence of
(SEQ ID NO: 1303)
5′-T005p001T005p001G004pC004pA004pG004pA007pU
004pG007pC007pU007pA004pG004pG004pU004pG004pU
004pU004pC004pA005pA005pX1085-3′;
and
(ii) an antisense strand (AS) comprising
a nucleotide sequence of
(SEQ ID NO: 1307)
5′-X033U1027p001U007p001G004pA004pA004pC007pA
004pC004pC004pU004pA004pG004pC004pA007pU004pC
007pU004pG004pC004pA004pA004p001A004p001C004-3′;

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; p001 is a phosphorothioate linkage; and X1085 is a ligand (L96),
wherein the ligand (L96) is

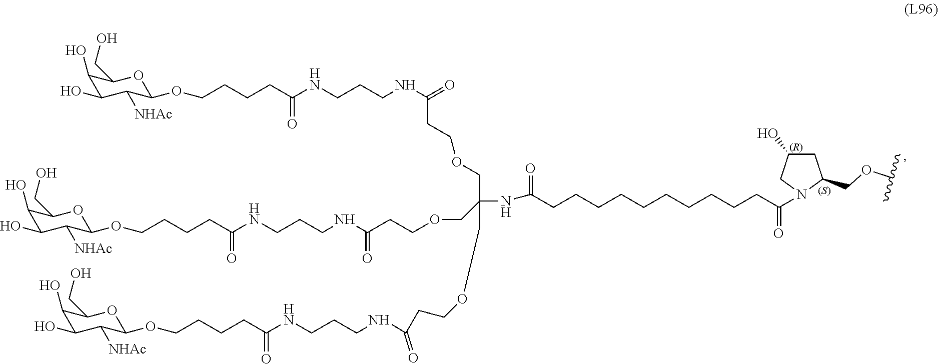

wherein the dsRNAi has the following schematic:

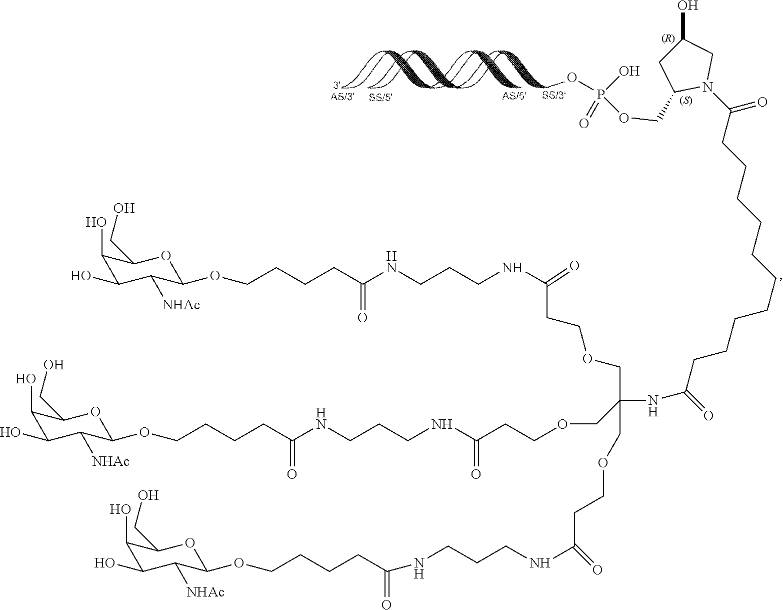

or a pharmaceutically acceptable salt thereof (e.g., sodium salt form).

In some embodiments, a double stranded RNAi agent includes:

(i) a sense strand (SS) consisting of
a nucleotide sequence of
(SEQ ID NO: 1303)
5′-T005p001T005p001G004pC004pA004pG004pA007pU
004pG007pC007pU007pA004pG004pG004pU004pG004pU
004pU004pC004pA005pA005px1085-3′;
and
(ii) an antisense strand (AS) consisting of
a nucleotide sequence of
(SEQ ID NO: 1307)
5′-X033U1027p001U007p001G004pA004pA004pC007pA
004pC004pC004pU004pA004pG004pC004pA007pU004pC
007pU004pG004pC004pA004pA004p001A004p001C004-3′;

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; p001 is a phosphorothioate linkage; and X1085 is a ligand (L96),
wherein the ligand (L96) is

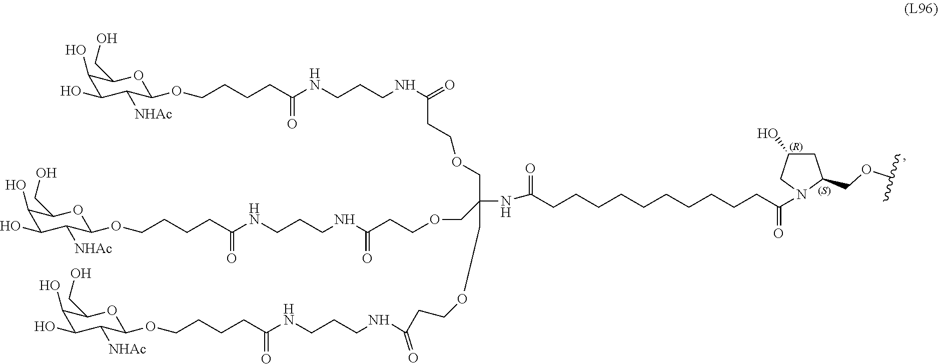

wherein the dsRNAi has the following schematic:

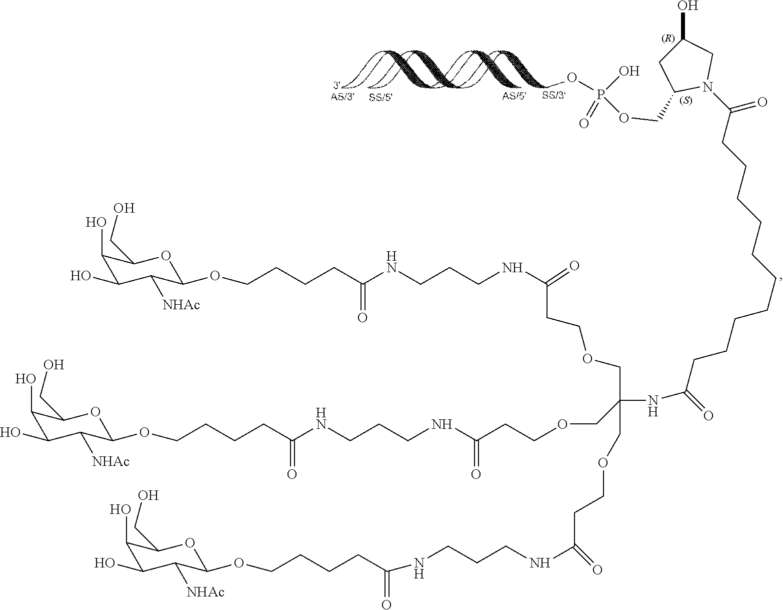

or a pharmaceutically acceptable salt thereof (e.g., sodium salt form).

In some embodiments, a double stranded RNAi agent (e.g., siRNA agent) includes:
(i) a sense strand (SS) comprising a nucleotide sequence of

(i) a sense strand (SS) comprising
a nucleotide sequence of
(SEQ ID NO: 1451)
5′-T005p001G005p001U004pA004pG004pC004pU007pA
004pC007pA007pA007pU004pG004pu004pU004pG004pU
004pC004pA004p001A005p001A005-3′;
(ii) an antisense strand (AS) comprising
a nucleotide sequence of
(SEQ ID NO: 1466)
5′-X033U1027p001U007p001U004pG004pA004pC007pA
004pA004pC004pA004pU004pU004pG004pU007pA004pG
007pC004pU004pA004pC004pA004p001G004p001A004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
wherein the ligand (L96) is

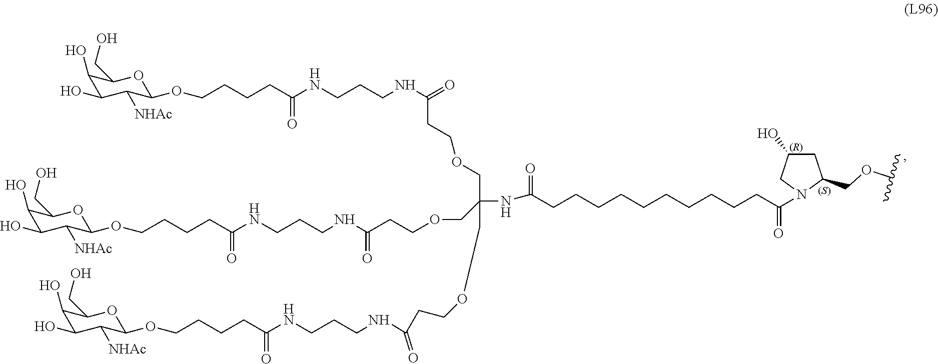

wherein the ligand is conjugated to 3′ end of the sense strand (SS), e.g., via a phosphodiester or phosphorothioate linkage, to form the following schematic:

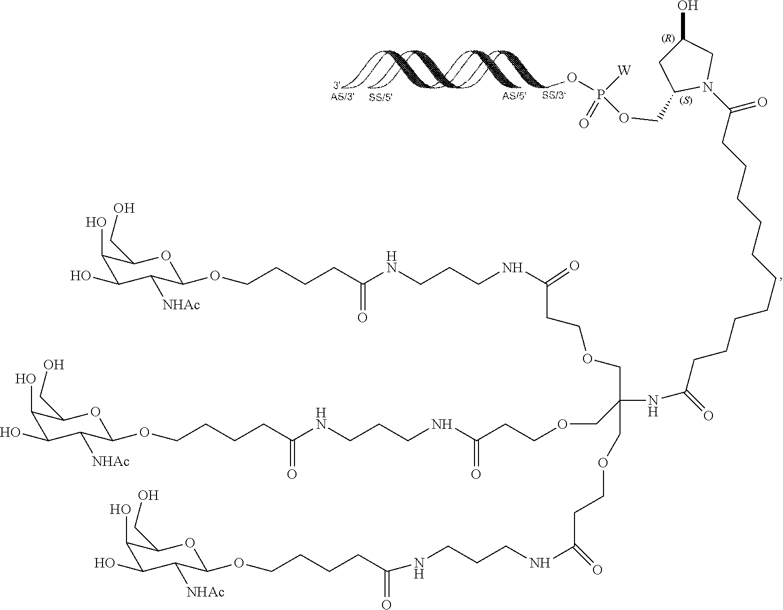

wherein W is —OH
   or a pharmaceutically acceptable salt thereof.

In some embodiments, a double stranded RNAi agent (e.g., siRNA agent) includes:

(i) a sense strand (SS) consisting of
a nucleotide sequence of
(SEQ ID NO: 1451)
5′-T005p001G005p001U004pA004pG004pC004pU007pA
004pC007pA007pA007pU004pG004pU004pU004pG004pU
004pC004pA004p001A005p001A005-3′;
(ii) an antisense strand (AS) consisting of
a nucleotide sequence of
(SEQ ID NO: 1466)
5′-X033U1027p001U007p001U004pG004pA004pC007pA
004pA004pC004pA004pU004pU004pG004pU007pA004pG
007pC004pU004pA004pC004pA004p001G004p001A004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
wherein the ligand (L96) is

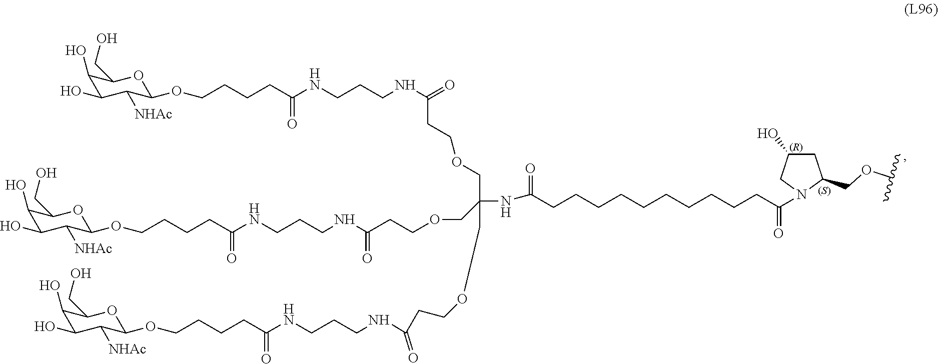

wherein the ligand is conjugated to 3′ end of the sense strand (SS), e.g., via a phosphodiester or phosphorothioate linkage, to form the following schematic:

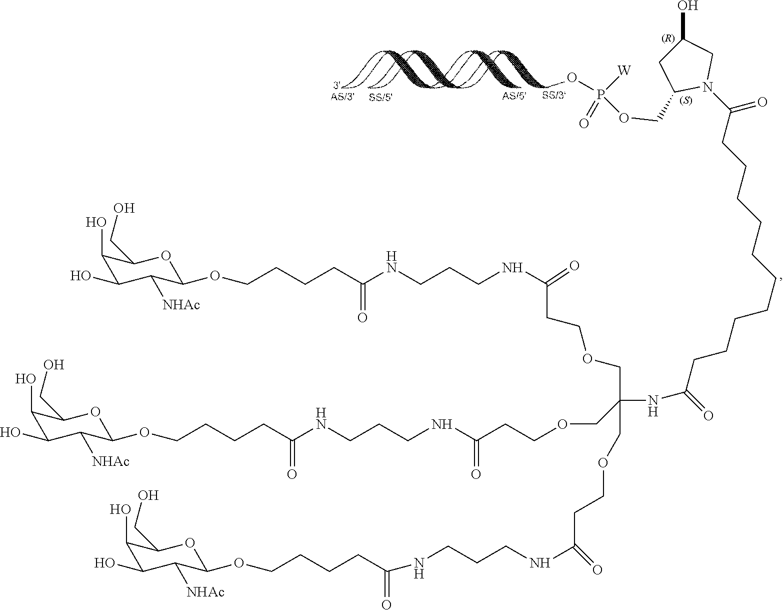

wherein W is —OH,
or a pharmaceutically acceptable salt thereof.

In some embodiments, a double stranded RNAi agent (e.g., siRNA agent) includes:

(i) a sense strand (SS) comprising
a nucleotide sequence of
(SEQ ID NO: 1451)
5′-T005p001G005p001U004pA004pG004pC004pU007pA
004pC007pA007pA007pU004pG004pU004pU004pG004pU
004pC004pA004p001A005p001A005-3′;
(ii) an antisense strand (AS) comprising
a nucleotide sequence of
(SEQ ID NO: 1466)
5′-X033U1027p001U007p001U004pG004pA004pC007pA
004pA004pC004pA004pU004pU004pG004pU007pA004pG
007pC004pU004pA004pC004pA004p001G004p001A004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
wherein the ligand (L96) is

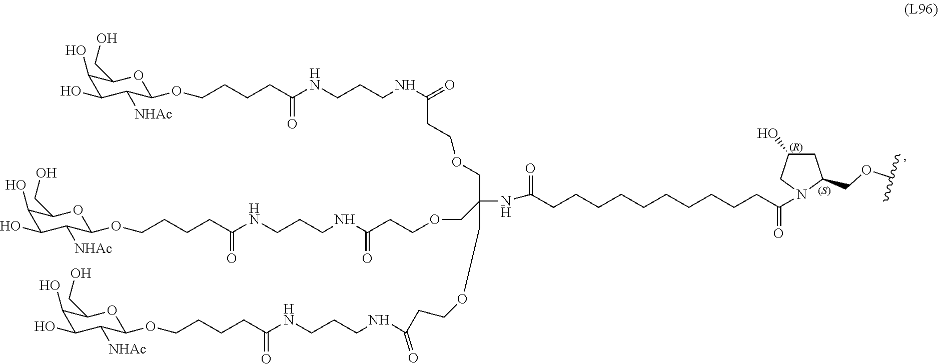

wherein the ligand is conjugated to 3′ end of the sense strand (SS) via a phosphodiester linkage to form the following schematic:

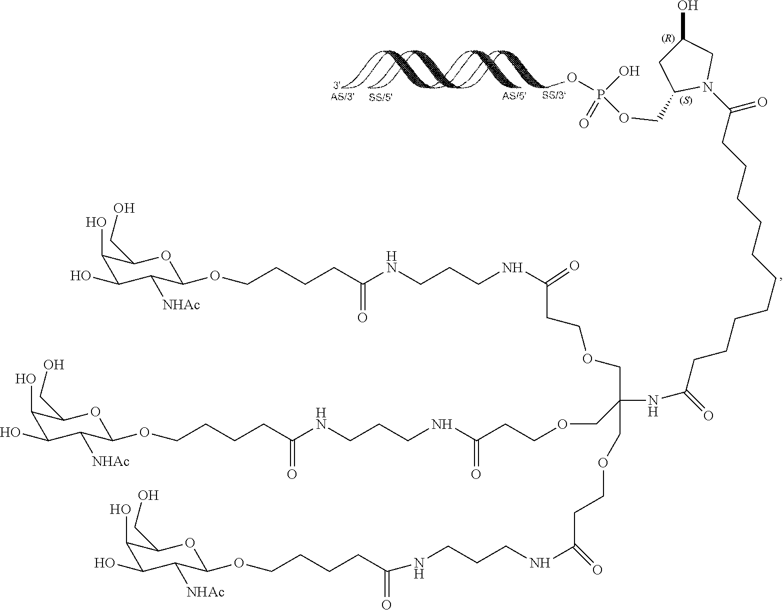

or a pharmaceutically acceptable salt thereof (e.g., sodium salt form).

In some embodiments, a double stranded RNAi agent (e.g., siRNA agent) includes:

(i) a sense strand (SS) consisting of
a nucleotide sequence of
(SEQ ID NO: 1451)
5′-T005p001G005p001U004pA004pG004pC004pU007pA
004pC007pA007pA007pU004pG004pU004pU004pG004pU
004pC004pA004p001A005p001A005-3′;
(ii) an antisense strand (AS) comprising
a nucleotide sequence of
(SEQ ID NO: 1466)
5′-X033U1027p001U007p001U004pG004pA004pC007pA
004pA004pC004pA004pU004pU004pG004pU007pA004pG
007pC004pU004pA004pC004pA004p001G004p001A004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; X033U1027 is 5′-(E)-vinylphosphonate-2′-O-methyluridine; p is a phosphodiester linkage; and p001 is a phosphorothioate linkage,
wherein the ligand (L96) is

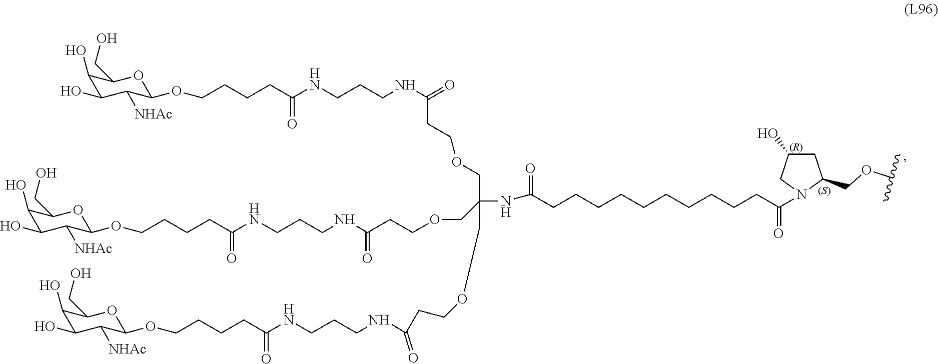

wherein the ligand is conjugated to 3′ end of the sense strand (SS) via a phosphodiester linkage to form the following schematic:

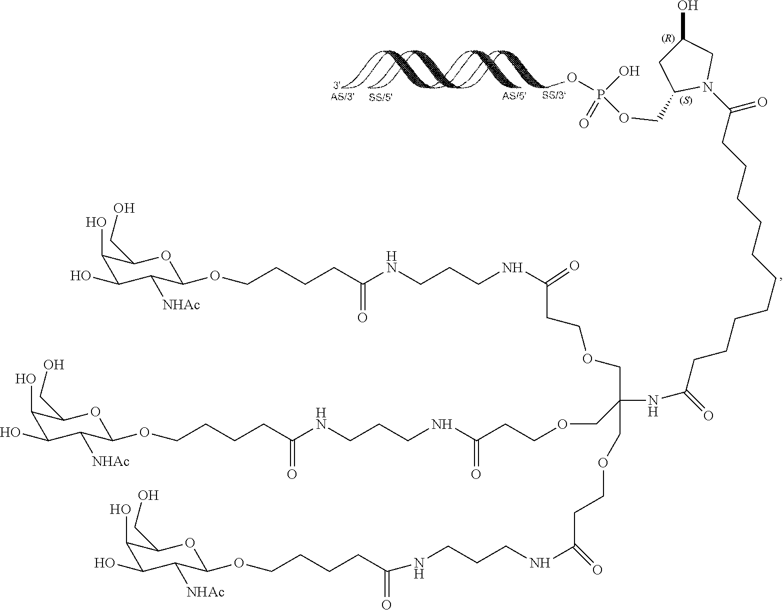

or a pharmaceutically acceptable salt thereof (e.g., sodium salt form).

In some embodiments, a double stranded RNAi agent (e.g., siRNA agent) includes:

(i) a sense strand (SS) comprising
a nucleotide sequence of
(SEQ ID NO: 1456)
5′-A005p001A005p001G004pG004pA004pC004pU007pA
004pA007pC007pA007pU004pA004pA004pA004pA004pU
004pC004pU004p001G005p001T005-3′;
(ii) an antisense strand (AS) comprising
a nucleotide sequence of
(SEQ ID NO: 2601)
5′-X033A1027p001C007p001A042pG004pA004pU007pU
004pU004pU004pA004pU004pG004pU004pU007pA004pG
007pU004pC004pC004pU004pU004p001U004p001A004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is TNA with adenine; X033A1027 is 5′-(E)-vinylphosphonate-2′-O-methyladenosine; p is a phosphodiester linkage; and p001 is a phosphorothioate linkage,
wherein the ligand (L96) is

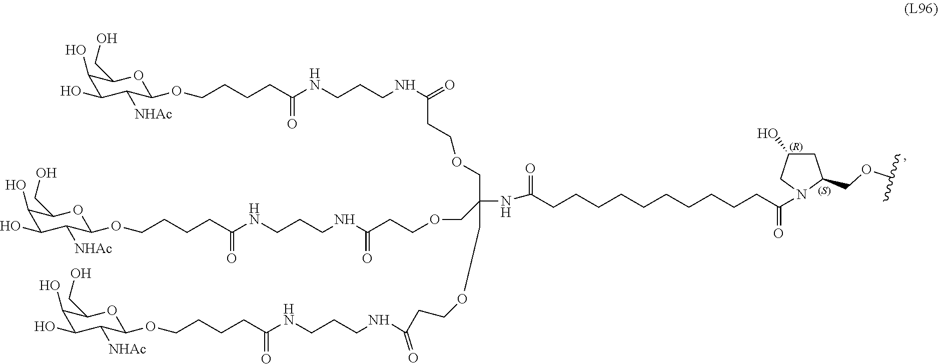

wherein the ligand is conjugated to 3′ end of the sense strand (SS), e.g., via a phosphodiester or phosphorothioate linkage, to form the following schematic:

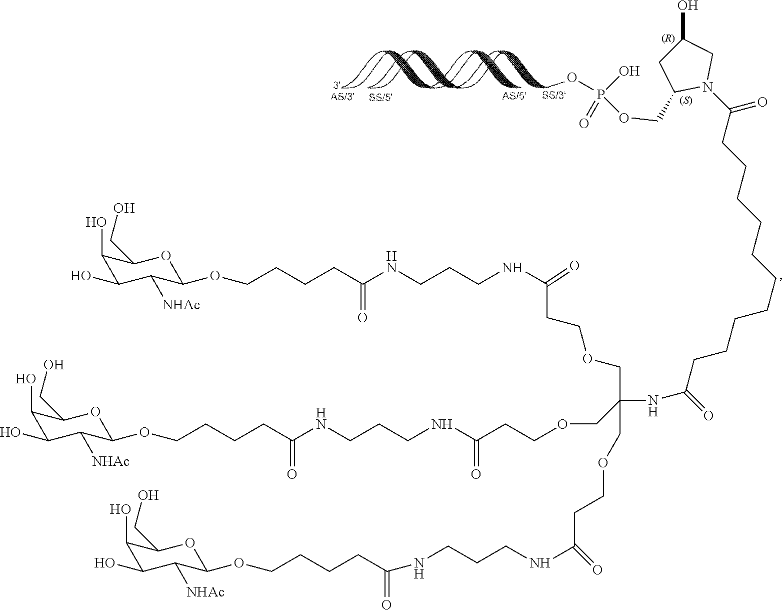

wherein W is —OH,
or a pharmaceutically acceptable salt thereof.

In some embodiments, a double stranded RNAi agent (e.g., siRNA agent) includes:

(1) a sense strand (SS) consisting of
a nucleotide sequence of
(SEQ ID NO: 1456)
5′-A005p001A005p001G004pG004pA004pC004pU007pA
004pA007pC007pA007pU004pA004pA004pA004pA004pU
004pC004pU004p001G005p001T005-3′;
(ii) an antisense strand (AS) consisting of
a nucleotide sequence of
(SEQ ID NO: 2601)
5′-X033A1027p001C007p001A042pG004pA004pU007pU
004pU004pU004pA004pU004pG004pU004pU007pA004pG
007pU004pC004pC004pU004pU004p001U004p001A004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is TNA with adenosine; X033A1027 is 5′-(E)-vinylphosphonate-2′-O-methyladenosine; p is a phosphodiester linkage; and p001 is a phosphorothioate linkage,
wherein the ligand (L96) is

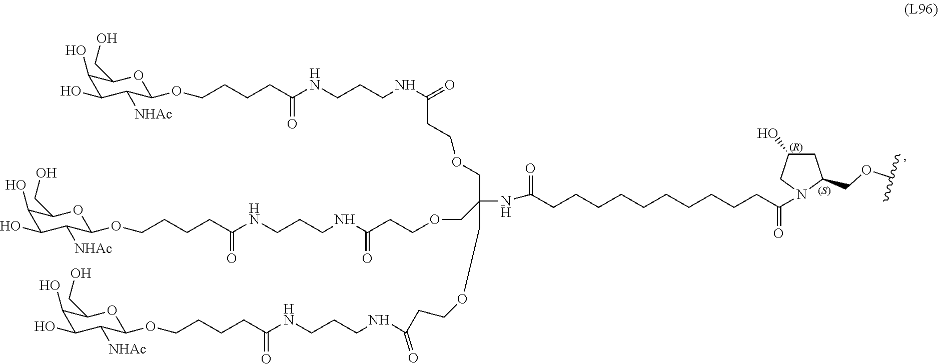

wherein the ligand is conjugated to 3′ end of the sense strand (SS), e.g., via a phosphodiester or phosphorothioate linkage, to form the following schematic:

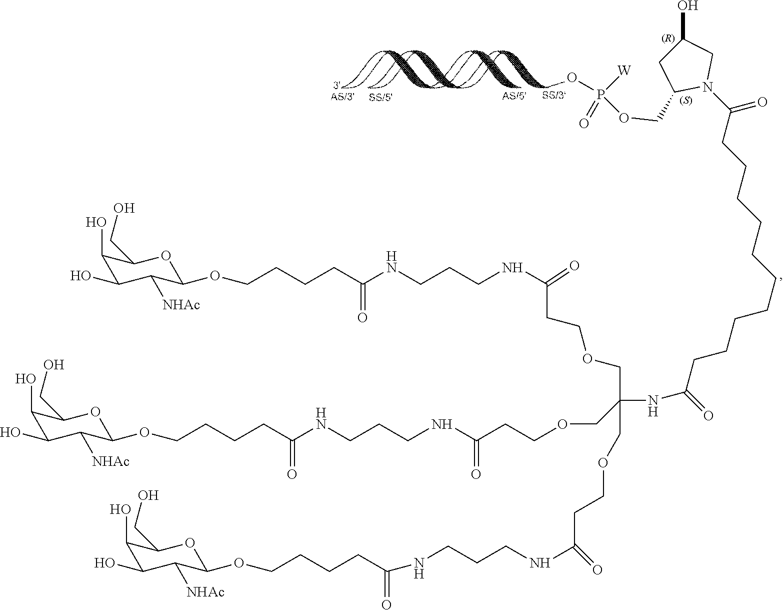

wherein W is —OH,
or a pharmaceutically acceptable salt thereof.

In some embodiments, a double stranded RNAi agent (e.g., siRNA agent) includes:

(i) a sense strand (SS) comprising
a nucleotide sequence of
(SEQ ID NO: 1456)
5′-A005p001A005p001G004pG004pA004pC004pU007pA
004pA007pC007pA007pU004pA004pA004pA004pA004pU
004pC004pU004p001G005p001T005-3′;
(ii) an antisense strand (AS) comprising
a nucleotide sequence of
(SEQ ID NO: 2601)
5′-X033A1027p001C007p001A042pG004pA004pU007pU
004pU004pU004pA004pU004pG004pU004pU007pA004pG
007pU004pC004pC004pU004pU004p001U004p001A004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is TNA with adenosine; X033A1027 is 5′-(E)-vinylphosphonate-2′-O-methyladenosine; p is a phosphodiester linkage; and p001 is a phosphorothioate linkage,
wherein the ligand (L96) is

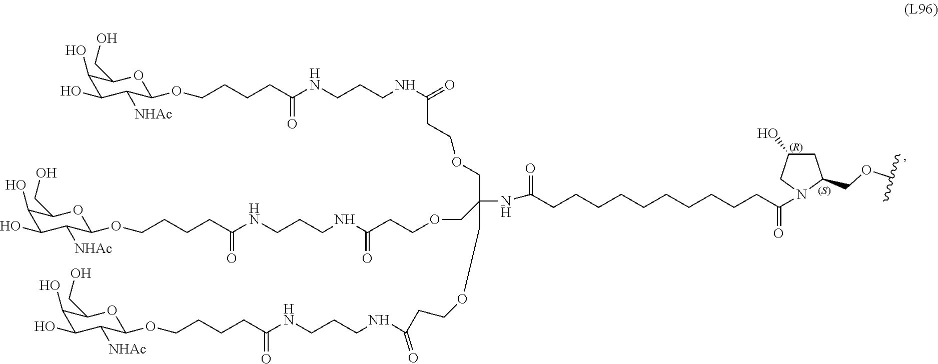

wherein the ligand is conjugated to 3′ end of the sense strand (SS) via a phosphodiester linkage to form the following schematic:

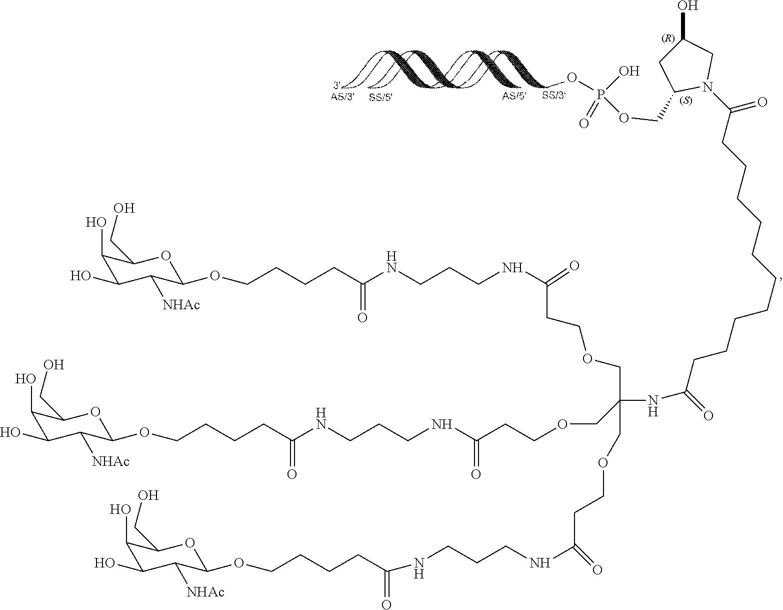

or a pharmaceutically acceptable salt thereof (e.g., sodium salt form).

In some embodiments, a double stranded RNAi agent (e.g., siRNA agent) includes:

(i) a sense strand (SS) consisting of
a nucleotide sequence of
(SEQ ID NO: 1456)
5′-A005p001A005p001G004pG004pA004pC004pU007pA
004pA007pC007pA007pU004pA004pA004pA004pA004pU
004pC004pU004p001G005p001T005-3′;
(ii) an antisense strand (AS) consisting of
a nucleotide sequence of
(SEQ ID NO: 2601)
5′-X033A1027p001C007p001A042pG004pA004pU007pU
004pU004pU004pA004pU004pG004pU004pU007pA004pG
007pU004pC004pC004pU004pU004p001U004p001A004-3′;
and
(iii) a ligand (L96),

wherein:
   A004 is 2′-O-methyladenosine; U004 is 2′-O-methyluridine; C004 is 2′-O-methylcytidine; G004 is 2′-O-methylguanosine; A005 is 2′-O-methoxyethyl(MOE) adenosine; T005 is 2′-O-methoxyethyl(MOE)thymidine (or 5-methyl uridine); C005* is 2′-O-methoxyethyl(MOE)5-methyl-cytidine; G005 is 2′-O-methoxyethyl (MOE)guanosine; A007 is 2′-fluoroadenosine; U007 is 2′-fluorouridine; C007 is 2′-fluorocytidine; G007 is 2′-fluoroguanosine; A042 is TNA with adenosine; X033A1027 is 5′-(E)-vinylphosphonate-2′-O-methyladenosine; p is a phosphodiester linkage; and p001 is a phosphorothioate linkage,
wherein the ligand (L96) is

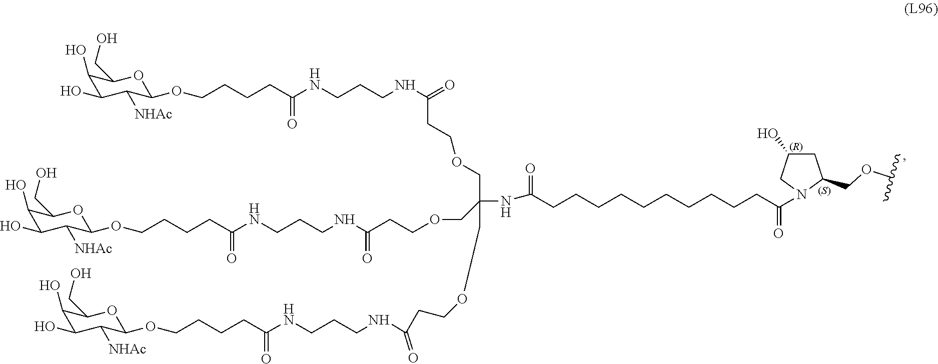

wherein the ligand is conjugated to 3′ end of the sense strand (SS) via a phosphodiester linkage to form the following schematic:

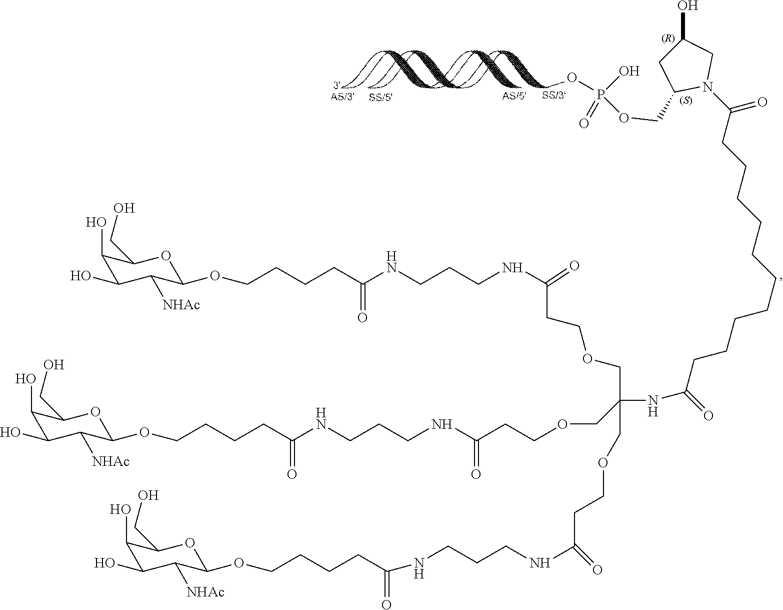

or a pharmaceutically acceptable salt thereof (e.g., sodium salt form).

Example compounds including the siRNAs and ligands as described herein are also listed in Table 5 below.

TABLE 5

| | Sequence | | |
|---|---|---|---|
| | Sense Strand | | Modification |
| Compound No. | Antisense Strand | Ligand | Pattern |
| 1 | SEQ ID NO: 1302 | L96 | D |
| | SEQ ID NO: 1306 | | |
| 2 | SEQ ID NO: 1303 | L96 | D |
| | SEQ ID NO: 1307 | | |
| 3 | SEQ ID NO: 1304 | L96 | E |
| | SEQ ID NO: 1308 | | |
| 4 | SEQ ID NO: 2613 | L96 | F |
| | SEQ ID NO: 2612 | | |
| 5 | SEQ ID NO: 284 | XC2000 | G-1 |
| | SEQ ID NO: 689 | | |
| 6 | SEQ ID NO: 284 | XC2000 | G-2 |
| | SEQ ID NO: 689 | | |
| 7 | SEQ ID NO: 284 | XC2000 | H |
| | SEQ ID NO: 689 | | |
| 8 | SEQ ID NO: 1295 | XC2000 | D |
| | SEQ ID NO: 1299 | | |
| 9 | SEQ ID NO: 284 | XC2001 | H |
| | SEQ ID NO: 689 | | |

### Combination

In an aspect, provided is a combination of a first agent (e.g., HMGCR inhibitor such as dsRNAi agent as described herein) and one or more additional therapeutic agents.

In an aspect, provided also is a combination of a first agent (e.g., HMGCR inhibitor such as dsRNAi agent as described herein) and a second agent. The term “additional therapeutic agent” and “second agent” may be interchangeably used in the context of their use in combination or combination therapy.

In certain aspects, the combination includes a first agent including the dsRNAi agent as described herein (e.g., HMGCR siRNA in Tables 1 to 5, 10 and 14-16) and a second agent. In certain aspects, the combination includes a first agent including one or more dsRNAi agents as described in WO2023/009687, WO2004/138105, and WO2024/260434.

The additional therapeutic agents suitable for the combination with the dsRNAi agents described herein may include a drug or agent that has been used or proven to be useful in treating a disorder of lipid metabolism (e.g., high cholesterol). In certain aspects, pharmaceutical compositions are formulated to administering the dsRNAi agents as described herein for the purpose of a combination with one or more additional therapeutic agents that has been used or proved to be useful in lowering LDL-C in a subject. In certain aspects, the additional therapeutic agent may suitably include selected from a HMGCR small molecule inhibitor (e.g., statins), a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acyl-CoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

In some embodiments, the additional therapeutic agent suitable for the combination with the dsRNAi agents described herein may include a lysophosphatidic acid (LPA) receptor inhibitor. In some embodiments, the additional therapeutic agent may include an angiotensinogen (AGT) inhibitor. In some embodiments, the additional therapeutic agent may include bile sequestering agents (e.g., cholestyramin E). In some embodiments, the additional therapeutic agent includes VLDL secretion inhibitors (e.g., niacin). In some embodiments, the additional therapeutic agent includes lipophilic antioxidants (e.g., Probucol). In some embodiments, the additional therapeutic agent includes acyl-CoA cholesterol acyl transferase inhibitors. In some embodiments, the additional therapeutic agent includes farnesoid X receptor antagonists. In some embodiments, the additional therapeutic agent includes sterol regulatory binding protein cleavage activating protein (SCAP) activators. In some embodiments, the additional therapeutic agent includes microsomal triglyceride transfer protein (MTP) inhibitors. In some embodiments, the additional therapeutic agent includes inhibitors to apolipoproteins (e.g., ApoA1, ApoB, ApoC3, ApoD, ApoE, ApoF, or ApoM). In some embodiments, the additional therapeutic agent includes and therapeutic antibodies against HMGCR. In some embodiments, the additional therapeutic agents may also include agents that raise high density lipoprotein (HDL). In some embodiments, the additional therapeutic agent includes such as cholesteryl ester transfer protein (CETP) inhibitors. In some embodiments, the additional therapeutic agents may also include dietary supplements, e.g., fish oil, and omega-3 oils. In some embodiments, the additional therapeutic agent does not include a HMGCR small molecule inhibitor (e.g., statins). In certain aspects, the additional therapeutic agent or the second agent is a second dsRNAi agent. In certain aspects, the additional therapeutic agent or the second agent is an antisense oligonucleotide (“ASO”) agent.

In some embodiments, the second dsRNAi agent is a dsRNAi agent or an ASO agent that targets one or more of the genes selected from the group consisting of PCSK9, LPA, AGT, ACE, ACE2, AGTR1, AGTR2, ApoA1, ApoB, ApoC3, ApoD, ApoE, ApoF, ApoM, ACAT, CETP, MTTP, PPAR, IBA T, FDFT1, ERG9, SQS1, Ccl2, CCR2, CCL7, CCL8, CCL13, and CCL16.

In some embodiments, the second agent is a second dsRNAi agent. In some embodiments, the second dsRNAi agent targets PCSK9 gene. In some embodiments, the second dsRNAi agent targets LPA gene. In some embodiments, the second dsRNAi agent targets AGT gene. In some embodiments, the second dsRNAi agent targets ACE gene. In some embodiments, the second dsRNAi agent targets ACE2 gene. In some embodiments, the second dsRNAi agent targets AGTR1 gene. In some embodiments, the second dsRNAi agent targets ApoA1 gene. In some embodiments, the second dsRNAi agent targets ApoB gene. In some embodiments, the second dsRNAi agent targets ApoC3 gene. In some embodiments, the second dsRNAi agent targets ApoD gene. In some embodiments, the second dsRNAi agent targets ApoE gene. In some embodiments, the second dsRNAi agent targets ApoF gene. In some embodiments, the second dsRNAi agent targets ApoMgene. In some embodiments, the second dsRNAi agent targets AGTR2 gene. In some embodiments, the second dsRNAi agent targets ACATgene. In some embodiments, the second dsRNAi agent targets CETP gene. In some embodiments, the second dsRNAi agent targets MTTP gene. In some embodiments, the second dsRNAi agent targets PPAR gene. In some embodiments, the second dsRNAi agent targets IBAT gene. In some embodiments, the second dsRNAi agent targets FDFT1 gene. In some embodiments, the second dsRNAi agent targets ERG9 gene. In some embodiments, the second dsRNAi agent targets SQS1 gene. In some embodiments, the second dsRNAi agent targets CCL2 gene. In some embodiments, the second dsRNAi agent targets CCR2 gene. In some embodiments, the second dsRNAi agent targets CCL7 gene. In some embodiments, the second dsRNAi agent targets CCL8 gene. In some embodiments, the second dsRNAi agent targets CCL13 gene. In some embodiments, the second dsRNAi agent targets CCL16 gene.

In some embodiments, the additional therapeutic agent includes the PCSK9 inhibitor. In some embodiments, the PCSK9 inhibitor may be a small molecule, antibody, peptide, or a therapeutic RNA interference agent (e.g., siRNA). In some embodiments, the additional therapeutic agent includes a dsRNAi agent (e.g., siRNA) targeting PCSK9. In some embodiments, the additional therapeutic agent is a second dsRNAi agent.

In some embodiments, the second dsRNAi agent includes inclisiran (e.g., Leqvio®).

In some embodiments, the second dsRNAi agent includes a sense strand having a nucleotide sequence of SEQ ID NO: 1478 (5′-csusagacCfuGfudTuugcuuuugu-3′) and an antisense strand having a nucleotide sequence of SEQ ID NO: 1479 (5′-asCfsaAfAfAfgCfaAfaAfcAfgGfuCfuagsasa-3′), wherein a, g, c and u are 2′-O-methyl (2′-OMe) A, G, C, or U; Af, Gf, Cf or Uf are 2′-fluoro A, G, C or U; dT is 2′-deoxythymidine; and s is a phosphorothioate linkage.

In some embodiments, the second dsRNAi agent includes a sense strand having a nucleotide sequence of SEQ ID NO: 1478 and an antisense strand having a nucleotide sequence of SEQ ID NO: 1479, wherein the sense strand is conjugated to L96 at 3′ end. In some embodiments, the second dsRNAi agent includes a sense strand having a nucleotide sequence of SEQ ID NO: 1480 and an antisense strand having a nucleotide sequence of SEQ ID NO: 1479.

In some embodiments, the second dsRNAi agent including a sense strand having a nucleotide sequence of SEQ ID NO: 1480 and an antisense strand having a nucleotide sequence of SEQ ID NO: 1479 is a pharmaceutically acceptable salt of inclisiran. In some embodiments, the second dsRNAi agent including a sense strand having a nucleotide sequence of SEQ ID NO: 1480 and an antisense strand having a nucleotide sequence of SEQ ID NO: 1479 is sodium salt of inclisiran.

TABLE 6a-1

| | Sequence (5′-3′) |
|---|---|
| SEQ ID: 1478 | 5′-csusagacCfuGfudTuugcuuuugu-3′ |
| (sense strand) | C004p001U004p001A004pG004pA004pC |
| | 004pC007pU004pG007pU004pT002pU00 |
| | 4pU004pG004pC004pU004pU004pU004p |
| | U004pG004pU004 |
| SEQ ID: 1480 | 5′-csusagacCfuGfudTuugcuuuugu-3′ |
| (sense strand) | C004p001U004p001A004pG004pA004p |
| | C004pC007pU004pG007pU004PT002pU |
| | 004pU004pG004pC004pU004pU004pU0 |
| | 04pU004pG004pU004pX1085 |
| SEQ ID: 1479 | 5′-asCfsaAfAfAfgCfaAfaAfcAfgGfuC |
| (antisense | fuagsasa-3′ |
| strand) | A004p001C007p001A004pA007pA007pA |
| | 007pG004pC007pA004pA007pA004pA00 |
| | 7pC004pA007pG004pG007pU004pC007p |
| | U004pA004pG004p001A004p001A004 |

Specific codes in the nucleotide sequences are indicated in above, e.g., Tables A and A-1.

Additional suitable second dsRNAi agent targeting PCSK9 in Table 6a-1, or variants thereof and synthesis thereof are also described in WO2014/089313, entire contents of which are incorporated herein by reference.

In some embodiment, the second dsRNAi agent may have a structure of

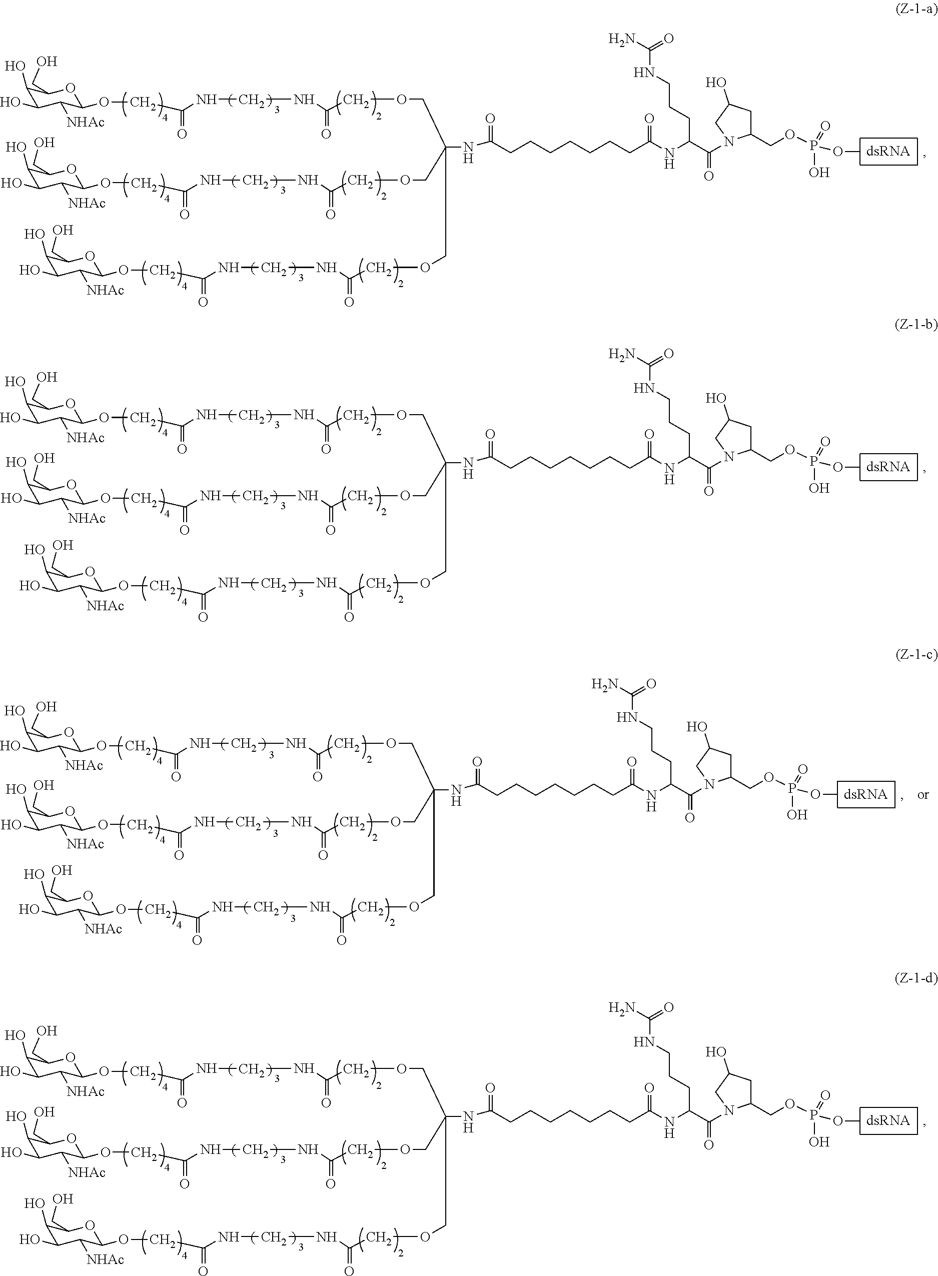

or a pharmaceutically acceptable salt thereof,
wherein the dsRNA includes any one of PCSK9 siRNA in Table 6a, the dsRNA includes:
   (i) a sense strand; and
   (ii) an antisense strand forming a duplex with the sense strand.

TABLE 6a-2

| PCSK9 | | | SEQ ID |
|---|---|---|---|
| SIRNA | Sequence (5′-3′) | Strand | NO |
| 1 | C004p0010004p001A004pG004pA004pC004pC007p0004pG007 | SS | 1488 |
| | pu004pT002pU004pU004pG004pC004pU004p0004pU004pU004 | | |
| | pG004pU004 | | |
| | A004p001C007p001A004pA007pA007pA007pG004pC007pA004 | AS | 1489 |
| | pA007pA004pA007pC004pA007pG004pG007p0004pC007pU004 | | |
| | pA004pG004p001A004p001A004 | | |
| 2 | C004p001U004pA004pG004pA004pC004pC007p0004pG007pU0 | SS | 1490 |
| | 04pT002pU004pU004pG004pC004p0004p0004pU004pU004pG0 | | |
| | 04pU004 | | |
| | A004p001C007p001A004pA007pA007pA007pG004pC007pA004 | AS | 1491 |
| | pA007pA004pA007pC004pA007pG004pG007p0004pC007p0004 | | |
| | pA004pG004p001A004p001A004 | | |
| 3 | C004p001U004p001A004pG004pA004pC004pC007pU004pG007 | SS | 1492 |
| | pU004pT002pU004pU004pG004pC004pU004pU004pU004pU004 | | |
| | pG004pU004 | | |
| | A004p001C007p001A004pA007pA007pA007pG004pC007pA004 | AS | 1493 |
| | pA007pA004pA007pC004pA007pG004pG007pU004pC007pU004 | | |
| | pA004pG004p001A004p001A004 | | |
| 4 | G004p001C004pC004pU004pG000pG000pA000pG000pU004pU0 | SS | 1494 |
| | 04pU004pA000pU004pU004pC004pG000pG000pA000pA000pT0 | | |
| | 02pT002 | | |
| | pU000pU000pC000pC000pG000pA000pA000pU000pA000pA000 | AS | 1495 |
| | pA000pC000pU000pC000pC000pA000pG000pG000pC000pT002 | | |
| | p001T002 | | |
| 5 | G000pC004pC004pU004pG000pG000pA000pG000pU004pU004p | SS | 1496 |
| | U004pA000pU004pU004pC004pG000pG000pA000pA000pT002p | | |
| | T002 | | |
| | pU004pU007pC004pC007pG004pA007pA004pU007pA004pA007 | AS | 1497 |
| | pA004pC007pU004pC007pC004pA007pG004pG007pC004pT002 | | |
| | pT002 | | |
| 6 | G000pC004pC004pU004pG000pG000pA000pG000pU004pU004p | SS | 1498 |
| | U004pA000pU004pU004pC004pG000pG000pA000pT002pT002 | | |
| | pU000pU000pC000pC000pG000pA000pA000pU000pA000pA000 | AS | 1499 |
| | pA000pC000pU000pC000pC000pA000pG000pG000pC000pT002 | | |
| | p001T002 | | |
| 7 | G000pC004pC004pU004pG000pG000pA000pG000pU004pU004p | SS | 1500 |
| | U004pA000pU004pU004pC004pG000pG000pA000pA000pT002p | | |
| | T002 | | |
| | pU000pU000pC000pC000pG000pA000pA000pU000pA000pA000 | AS | 1501 |
| | pA000pC000pU000pC000pC000pA000pG000pG000pC000pT002 | | |
| | p001T002 | | |
| 8 | G000pC004pC004pU004pG000pG000pA000pG000pU004pU004p | SS | 1502 |
| | U004pA000pU004pU004pC004pG000pG000pA000pA000pT002p | | |
| | T002 | | |
| | pU000pU000pC000pC000pG000pA000pA000pU000pA000pA000 | AS | 1503 |
| | pA000pC000pU000pC000pC000pA000pG000pG000pC000pT002 | | |
| | p001T002 | | |
| 9 | G000pC004pC004pU004pG000pG000pA000pG000pU004pU004p | SS | 1504 |
| | U004pA000pU004pU004pC004pG000pG000pA000pA000pT002p | | |
| | T002 | | |
| | pU004pU007pC004pC007pG004pA007pA004pU007pA004pA007 | AS | 1505 |
| | pA004pC007pU004pC007pC004pA007pG004pG007pC004pT002 | | |
| | pT002 | | |
| 10 | G000pC004pC004pU004pG000pG000pA000pG000pU004pU004p | SS | 1506 |
| | U004pA000pU004pU004pC004pG000pG000pA000pA000pT002p | | |
| | 001T002 | | |
| | pU000pU000pC000pC000pG000pA000pA000pU000pA000pA000 | AS | 1507 |
| | pA000pC000pU000pC000pC000pA000pG000pG000pC000pT002 | | |
| | p001T002 | | |
| 11 | G000pC004pC004pU004pG000pG000pA000pG000pU004pU004p | SS | 1508 |
| | U004pA000pU004pU004pC004pG000pG000pA000pA000pT002p | | |
| | 001T002 | | |
| | G000pC004pC004pU004pG000pG000pA000pG000pU004pU004p | AS | 1509 |
| | U004pA000pU004pU004pC004pG000pG000pA000pA000pT002p | | |
| | T002 | | |
| 12 | C004pU004pG007pU004pG007pC004pU004pA007pG007pC004p | SS | 1510 |
| | A002pA007pC004pA007pC004pC004pC004pA004pA004 | | |
| | U004p001U007p001G004pG007pG004pU007pG004pU004pU004 | AS | 1511 |
| | pG004pC007pU007pA004pG007pC004pA007pC004pA007pG004 | | |
| | p001C007p001C004 | | |
| 13 | C004pU004pG007pU004pG007pC004pU004pA007pG007pC004p | SS | 1512 |
| | A002pA007pC004pA004pC004pC004pC004pA004pA004 | | |
| | U004p001U007p001G004G007G004U007G004U004U004G004C0 | AS | 1513 |
| | 07U007A004G005C004A007C004A007G004p001C007p001C004 | | |
| 14 | C004pU004pG007pU004pG007pC004pU004pA007pG007pC004p | SS | 1514 |
| | A002pA007pC004pA004pC004pC004pC004pA004pA004 | | |
| | U004p001U007p001G004pG007pG004pU007pG004pU004pU004 | AS | 1515 |
| | pG004pC007pU007pA004pG007pC004pA007pC004pA007pG004 | | |
| | p001C007p001C004 | | |
| 15 | C004pU004pG007pU004pG007pC004pU004pA007pG007pC004p | SS | 1516 |
| | A002pA007pC004pA004pC004pC004pC004pA004pA004 | | |
| | U004p001U007pG004pG007pG004pU007pG004pU004pU004pG0 | AS | 1517 |
| | 04pC007pU007pA004pG007pC004pA007pC004pA007pG004p00 | | |
| | 1C007p001C004 | | |
| 16 | C004pU004pG007pU004pG007pC004pU004pA007pG007pC004p | SS | 1518 |
| | A002pA007pC004pA004pC004pC004pC004pA004pA004 | | |
| | U004p001U007pG004pG007pG004pU007pG004pU004pU004pG0 | AS | 1519 |
| | 04pC007pU007pA004pG007pC004pA007pC004pA007pG004pC0 | | |
| | 07p001C004 | | |
| 17 | C000pU000pG000pU000pG000pC000pU000pA000pG000pC000p | SS | 1520 |
| | A002pA000pC000pA000pC000pC000pC000pA000pA000 | | |
| | U007pU007pG007pG004pG007pU004pG007pU004pU004pG004p | AS | 1521 |
| | C007pU004pA007pG007pC007pA004pC007pA004pG007pC004p | | |
| | C007 | | |
| 18 | C000pU000pG000pU000pG000pC000pU000pA000pG000pC000p | SS | 1522 |
| | A002pA000pC000pA000pC000pC000pC000pA000pA000 | | |
| | U004U007G007G004G007U004G007U004U004G004C007U004A0 | AS | 1523 |
| | 07G007C007A004C007A004G007C004C007 | | |
| 19 | C000pU000pG000pU000pG000pC000pU000pA000pG000pC000p | SS | 1524 |
| | A002pA000pC000pA000pC000pC000pC000pA000pA000 | | |
| | U007pU004pG007pG004pG007pU004pG007pU004pU004pG004p | AS | 1525 |
| | C007pU004pA007pG007pC007pA004pC007pA004pG007pC004p | | |
| | C007 | | |
| 20 | C000pU000pG000pU000pG000pC000pU000pA000pG000pC000p | SS | 1526 |
| | A002pA000pC000pA000pC000pC000pC000pA000pA000 | | |
| | U007pU007pG004pG004pG007pU004pG007pU004pU004pG004p | AS | 1527 |
| | C007pU004pA007pG007pC007pA004pC007pA004pG007pC004p | | |
| | C007 | | |
| 21 | C000pU000pG000pU000pG000pC000pU000pA000pG000pC000p | SS | 1528 |
| | A002pA000pC000pA000pC000pC000pC000pA000pA000 | | |
| | U007pU007pG007pG004pG004pU004pG007pU004pU004pG004p | AS1 | 1529 |
| | C007pU004pA007pG007pC007pA004pC007pA004pG007pC004p | | |
| | C007 | | |
| 22 | C000pU000pG000pU000pG000pC000pU000pA000pG000pC000p | SS | 1530 |
| | A002pA000pC000pA000pC000pC000pC000pA000pA000 | | |
| | U007pU007pG007pG004pG007pU004pG004pU004pU004pG004p | AS | 1531 |
| | C007pU004pA007pG007pC007pA004pC007pA004pG007pC004p | | |
| | C007 | | |
| 23 | C000pU000pG000pU000pG000pC000pU000pA000pG000pC000p | SS | 1532 |
| | A002pA000pC000pA000pC000pC000pC000pA000pA000 | | |
| | U007pU007pG007pG004pG007pU004pG007pU004pU004pG004p | AS | 1533 |
| | C004pU004pA007pG007pC007pA004pC007pA004pG007pC004p | | |
| | C007 | | |
| 24 | C000pU000pG000pU000pG000pC000pU000pA000pG000pC000p | SS | 1534 |
| | A002pA000pC000pA000pC000pC000pC000pA000pA000 | | |
| | U007pU007pG007pG004pG007pU004pG007pU004pU004pG004p | AS | 1535 |
| | C007pU004pA004pG007pC007pA004pC007pA004pG007pC004p | | |
| | C007 | | |
| 25 | C000pU000pG000pU000pG000pC000pU000pA000pG000pC000p | SS | 1536 |
| | A002pA000pC000pA000pC000pC000pC000pA000pA000 | | |
| | U007pU007pG007pG004pG007pU004pG007pU004pU004pG004p | AS | 1537 |
| | C007pU004pA007pG004pC007pA004pC007pA004pG007pC004p | | |
| | C007 | | |
| 26 | C000pU000pG000pU000pG000pC000pU000pA000pG000pC000p | SS | 1538 |
| | A002pA000pC000pA000pC000pC000pC000pA000pA000 | | |
| | U007pU007pG007pG004pG007pU004pG007pU004pU004pG004p | AS | 1539 |
| | C007pU004pA007pG007pC004pA004pC007pA004pG007pC004p | | |
| | C007 | | |
| 27 | C000pU000pG000pU000pG000pC000pU000pA000pG000pC000p | SS | 1540 |
| | A002pA000pC000pA000pC000pC000pC000pA000pA000 | | |
| | U007pU007pG007pG004pG007pU004pG007pU004pU004pG004p | AS | 1541 |
| | C007pU004pA007pG007pC007pA004pC004pA004pG007pC004p | | |
| | C007 | | |
| 28 | C000pU000pG000pU000pG000pC000pU000pA000pG000pC000p | SS | 1542 |
| | A002pA000pC000pA000pC000pC000pC000pA000pA000 | | |
| | U007pU007pG007pG004pG007pU004pG007pU004pU004pG004p | AS | 1543 |
| | C007pU004pA007pG007pC007pA004pC007pA004pG004pC004C | | |
| | 007 | | |
| 29 | C000pU000pG000pU000pG000pC000pU000pA000pG000pC000p | SS | 1544 |
| | A002pA000pC000pA000pC000pC000pC000pA000pA000 | | |
| | U007pU007pG007pG004pG007pU004pG007pU004pU004pG004p | AS | 1545 |
| | C007pU004pA007pG007pC007pA004pC007pA004pG007pC004p | | |
| | C004 | | |
| 30 | C004p001U004p001A004G004A004C004C007U004G007U004T0 | SS | 1546 |
| | 02U004U004G004C004U004U004U004U004G004U004 | | |
| | A004p001C004p001A004pA007pA004pA007pG004pC007pA004 | AS | 1547 |
| | pA007pA004pA007pC004pA004pG004pG004pU004pC004pU004 | | |
| | pA004pG004p001A004p001A004 | | |
| 31 | C004p001U004p001A004pG004pA004pC004pC007pU004pG007 | SS | 1548 |
| | pU004PT002pU004pU004pG004pC004pU004pU004pU004pU004 | | |
| | pG004pU004 | | |
| | A004p001C007p001A004A000p001pA004pA007pG004pC007pA | AS | 1549 |
| | 004pA007pA004pA007pC004pA004pG004pG004pU004pC004pU | | |
| | 004pA004pG004p001A004p001A004 | | |
| 32 | C004p001U004p001A004pG004pA004pC004pC007pU004pG007 | SS | 1550 |
| | pU004PT002pU004pU004pG004pC004pU004pU004pU004pU004 | | |
| | pG004pU004 | | |
| | A004p001C004p001A004pA007pA004pA007pG004pC007pA004 | AS | 1551 |
| | pA007pA004pA007pC004pA007pG004pG004pU004pC004pU004 | | |
| | pA004pG004p001A004p001A004 | | |
| 33 | C004p001U004p001A004pG004pA004pC004pC007pU004pG007 | SS | 1552 |
| | pU004PT002pU004pU004pG004pC004pU004pU004pU004pU004 | | |
| | pG004pU004 | | |
| | A004p001C004p001A004A007A004A007G004C007A004A007A0 | AS | 1553 |
| | 04A007C004A004G004G007U004C004U004A004G004p001A004 | | |
| | p001A004 | | |
| 34 | C004p001U004p001A004pG004pA004pC004pC007pU004pG007 | SS | 1554 |
| | pU004pT002pU004pU004pG004pC004pU004pU004pU004pU004 | | |
| | pG004pU004 | | |
| | A004p001C004p001A004pA007pA004pA007pG004pC007pA004 | AS | 1555 |
| | pA007pA004pA007pC004pA004pG004pG004pU004pC007pU004 | | |
| | pA004pG004p001A004p001A004 | | |

Specific codes in the nucleotide sequences are indicated in above, e.g., Tables A and A-1.

Additional suitable second dsRNAi agent targeting PCSK9 in Table 6a-2, or variants thereof and synthesis thereof are also described in WO2022/266753, entire contents of which are incorporated herein by reference.

In some embodiment, the second dsRNAi agent may have a structure of

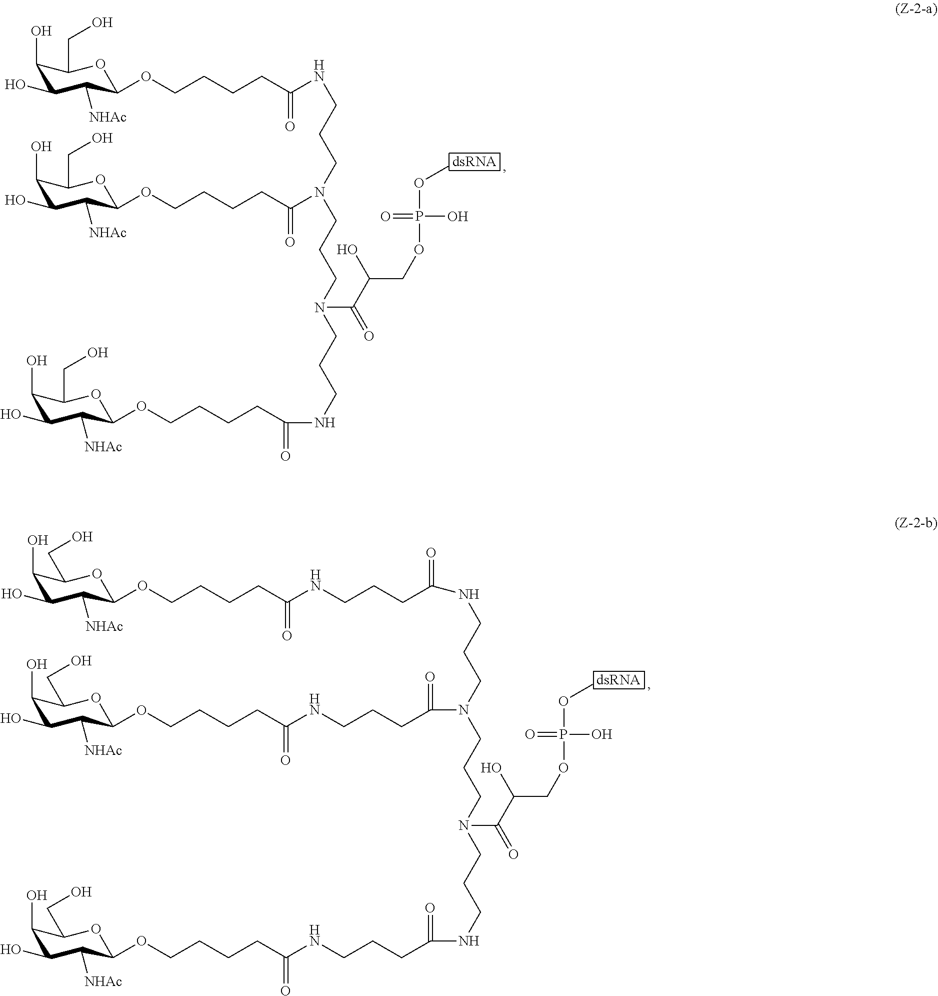

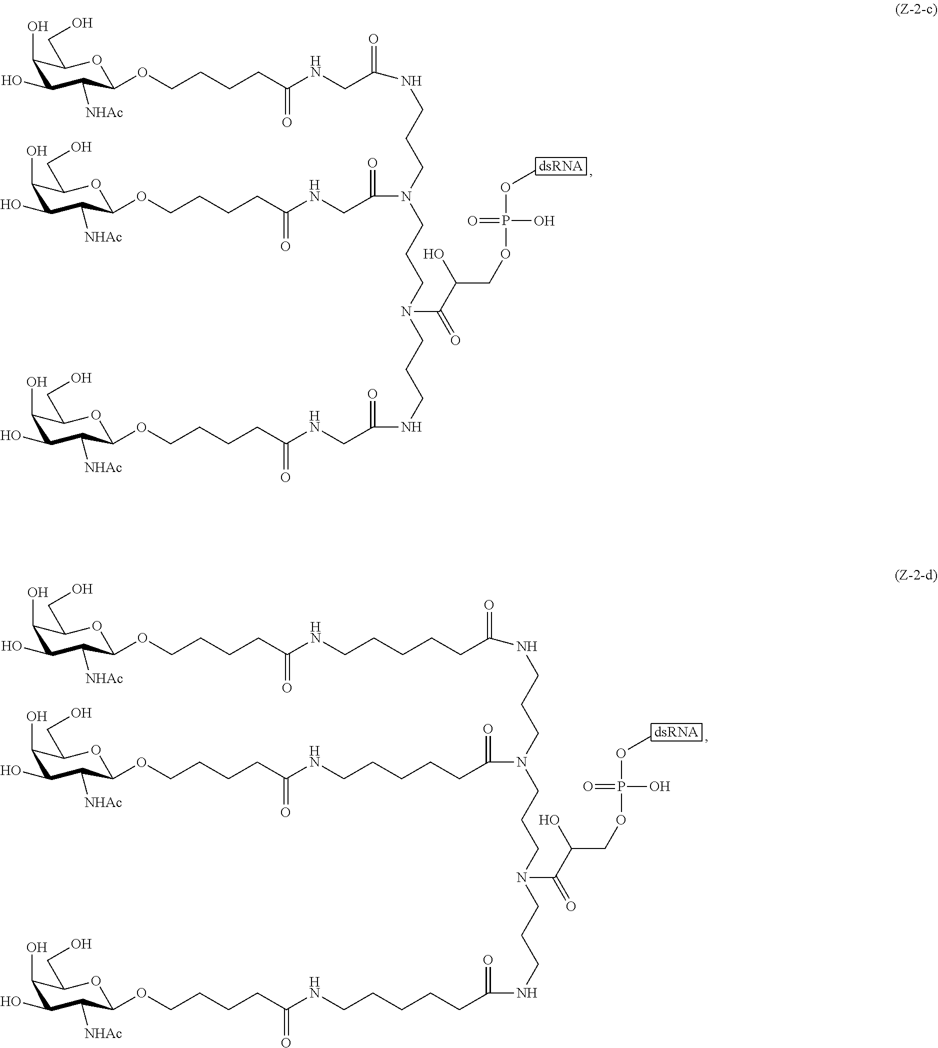

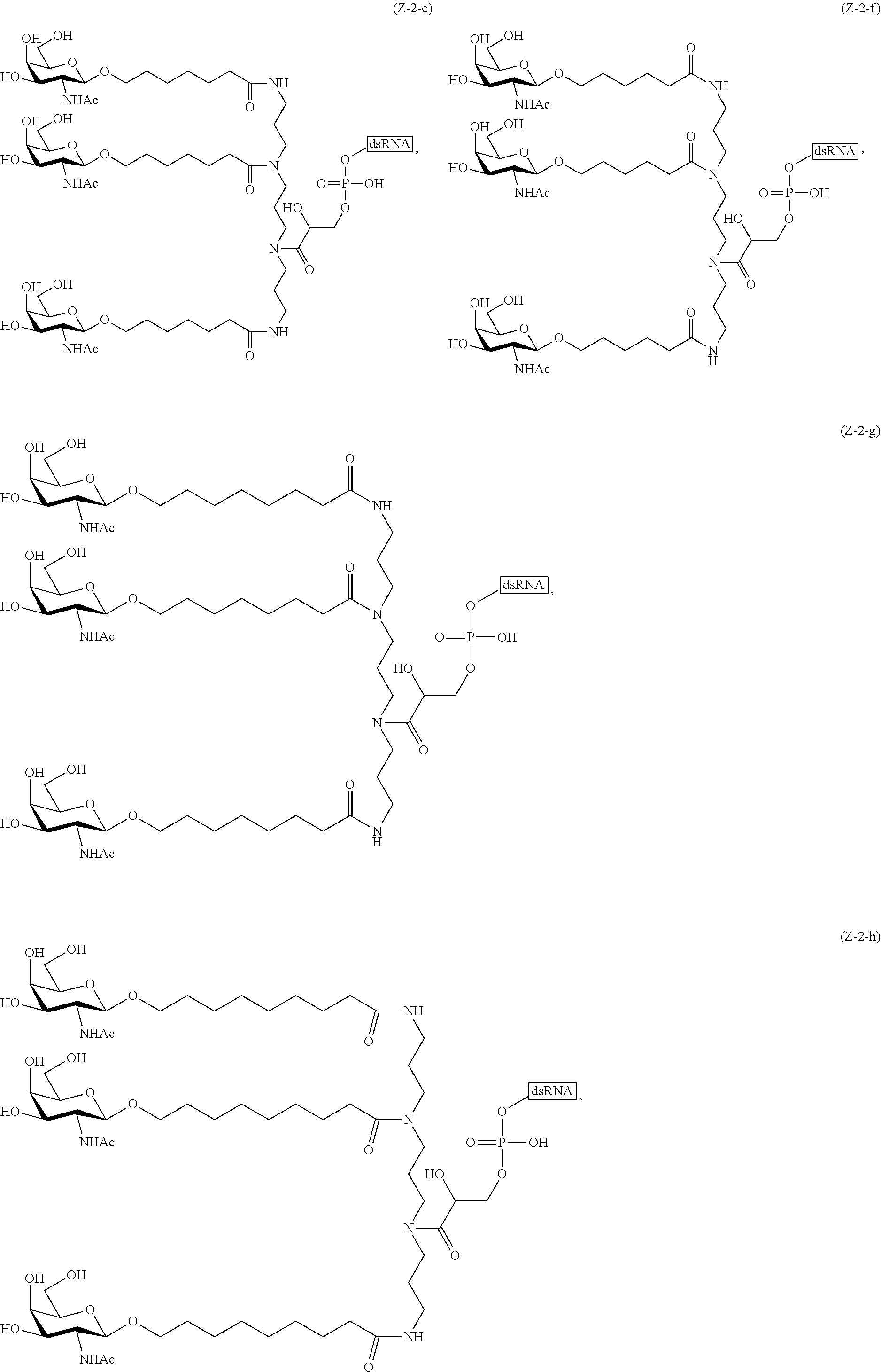

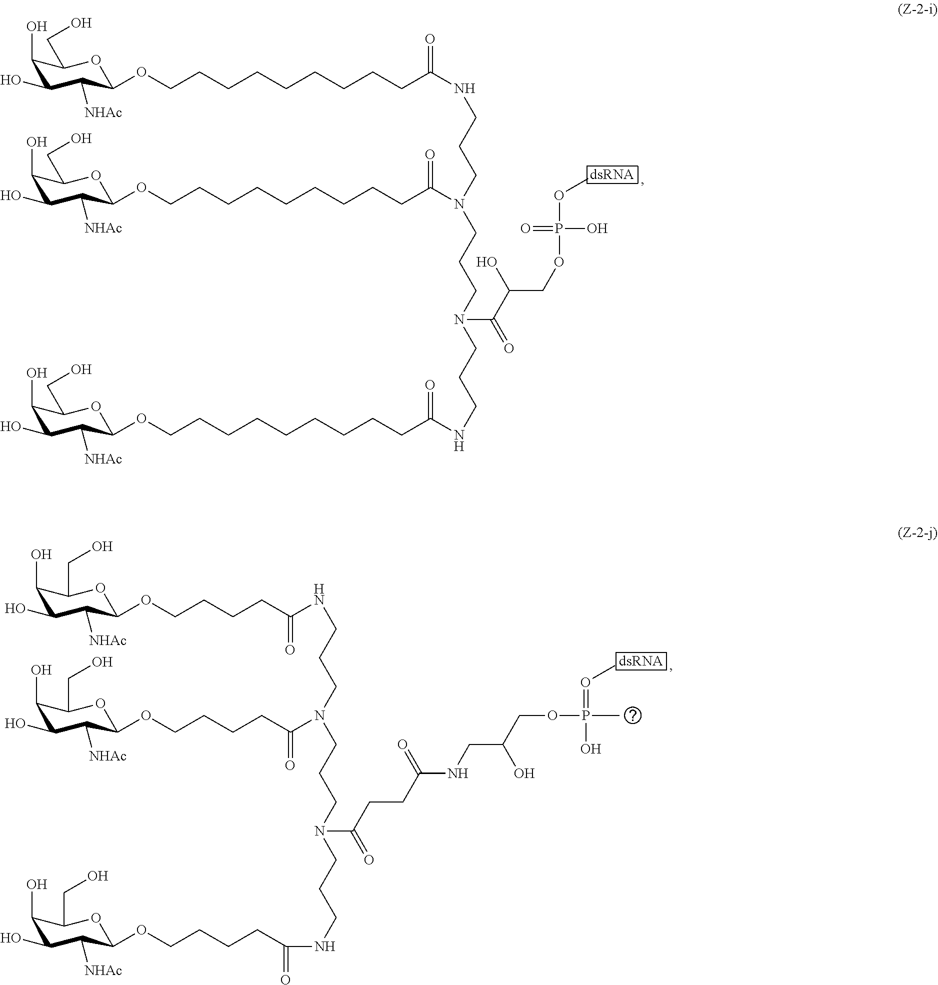

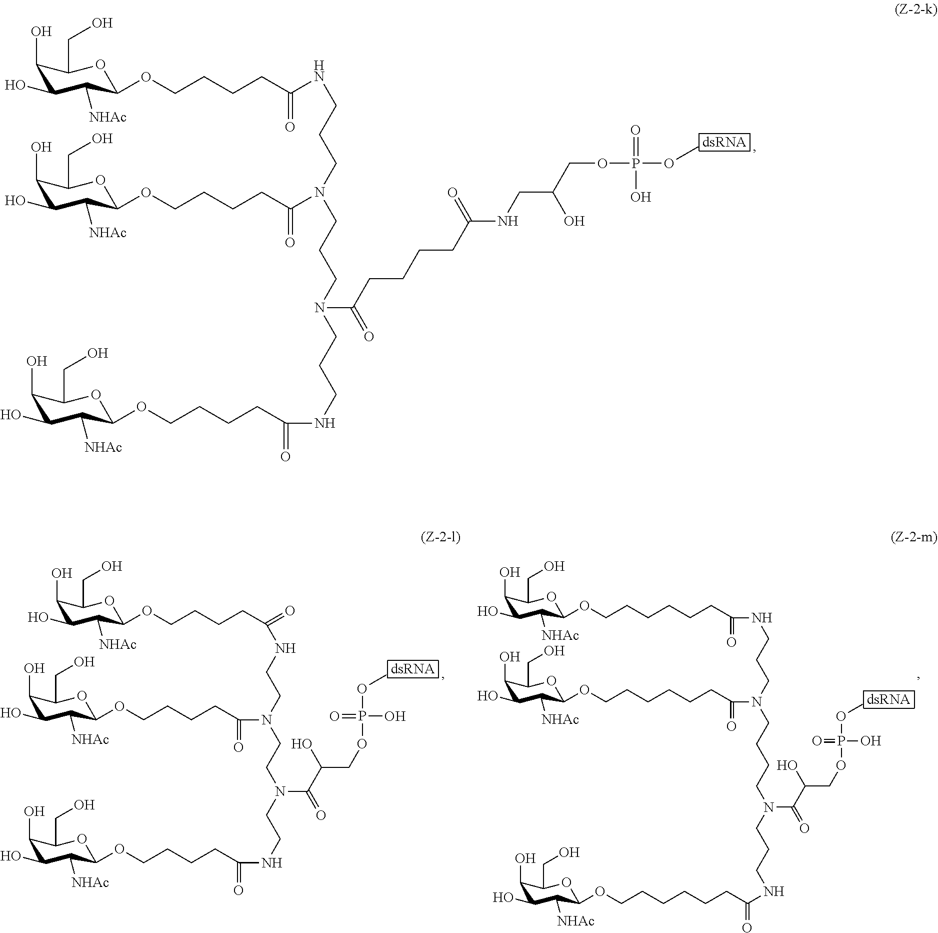

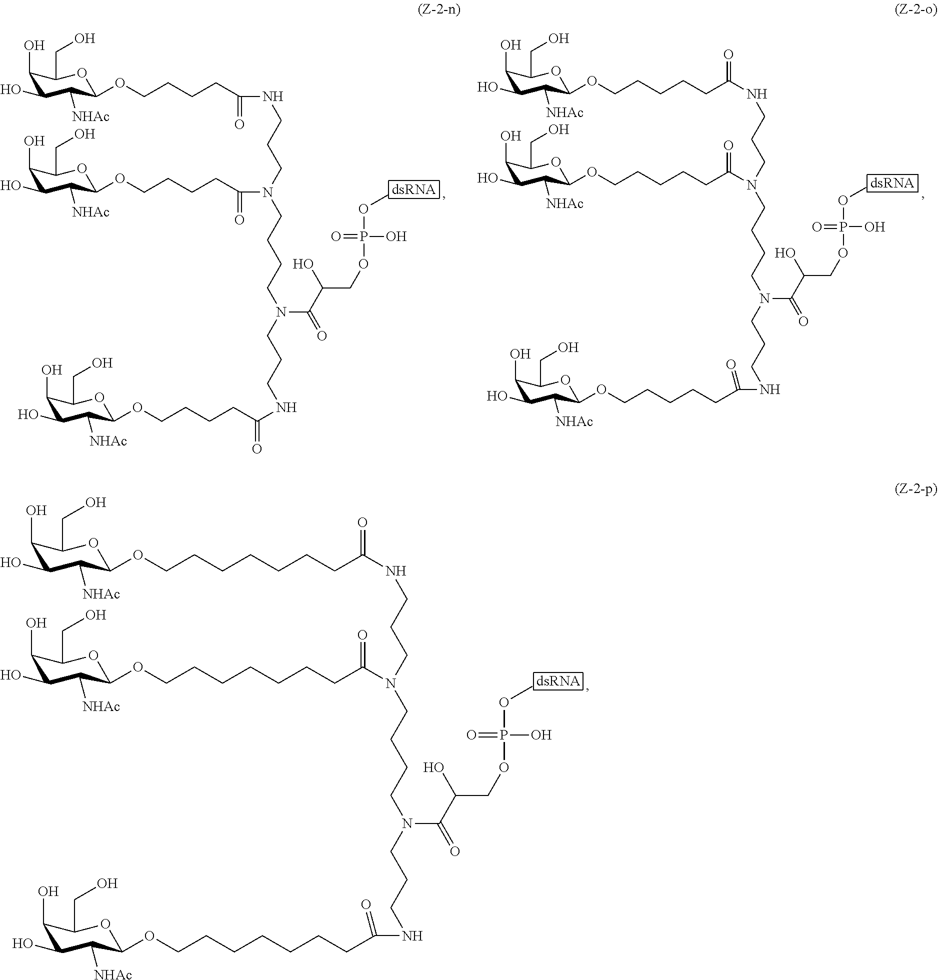

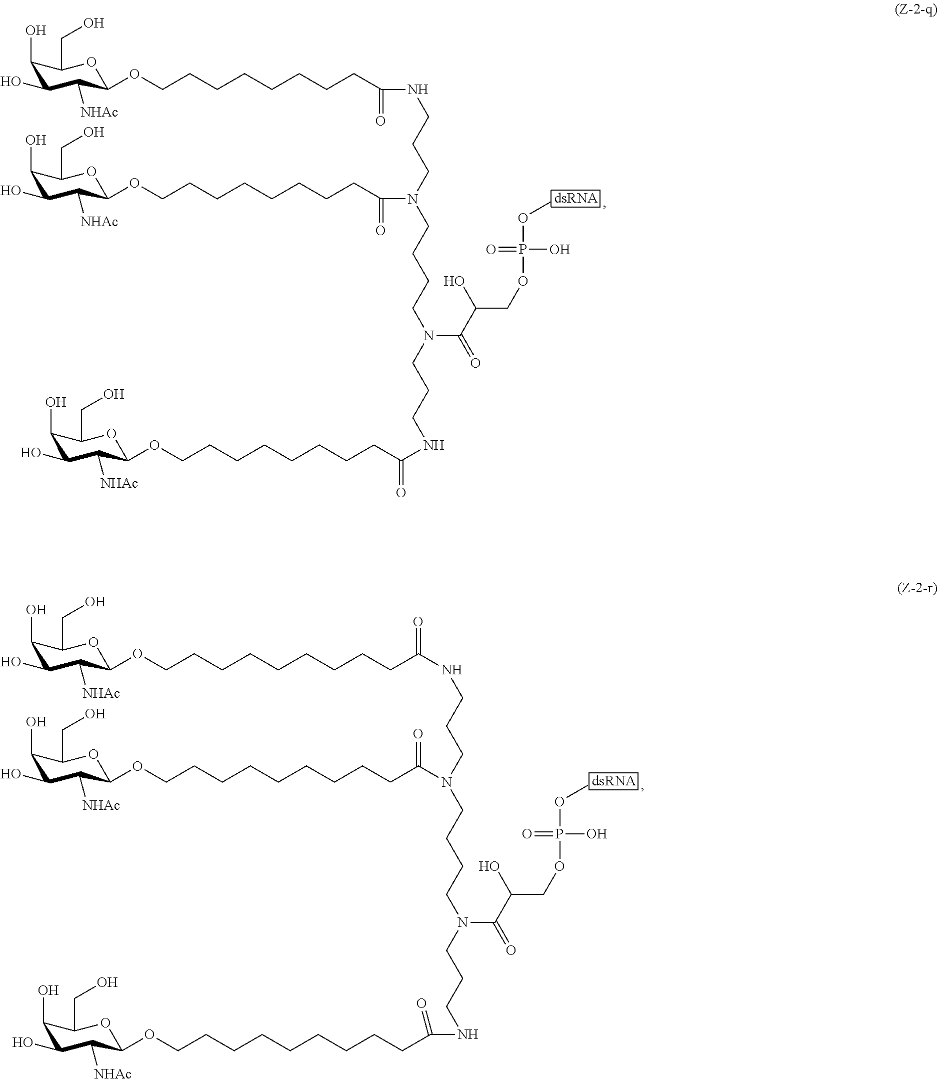

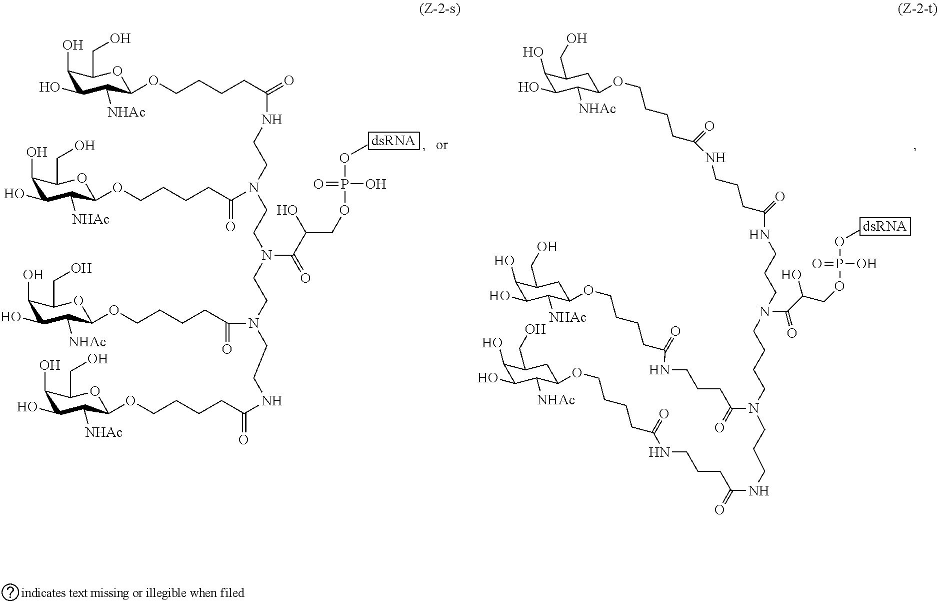

wherein the dsRNA includes any one of PCSK9 siRNA in Table 6b, the dsRNA includes.
(i) a sense strand; and
(ii) an antisense strand forming a duplex with the sense strand.

TABLE 6b

| PCSK9 | | | SEQ ID |
|---|---|---|---|
| SIRNA | Sequence (5′-3′) | Strand | NO |
| 35 | A000pA000pG000pC000pA000pA000pG000pC000pA000pG000p | SS | 1556 |
| | A000pC000pA000pU000pU000pU000pA000pU000pC000 | | |
| | G000pA000pU000pA000pA000pA000pU000pG000pU000pC000p | AS | 1557 |
| | U000pG000pC000pU000pU000pG000pC000pU000pU000pG000p | | |
| | G000 | | |
| 36 | C000pC000pA000pA000pG000pC000pA000pA000pG000pC000p | SS | 1558 |
| | A000pG000pA000pC000pA000pU000pU000pU000pA000pU000p | | |
| | C000 | | |
| | G000pA000pU000pA000pA000pA000pU000pG000pU000pC000p | AS | 1559 |
| | U000pG000pC000pU000pU000pG000pC000pU000pU000pG000p | | |
| | G000pG000pU000 | | |
| 37 | A004pA004pG004pC004pA004pA004pG007pC007pA007pG004p | SS | 1560 |
| | A004pC004pA004pU004pU004pU004pA004pU004pC004 | | |
| | G004pA007pU004pA004pA004pA007pU004pG004pU004pC004p | AS | 1561 |
| | U004pG004pC004pU007pU004pG007pC004pU004pU004pG004p | | |
| | G004 | | |
| 38 | A004pA004pG004pC004pA007pA004pG007pC007pA007pG004p | SS | 1562 |
| | A004pC004pA004pU004pU004pU004pA004pU004pC004 | | |
| | G004pA007pU004pA004pA004pA007pU004pG007pU007pC004p | AS | 1563 |
| | U004pG004pC004pU007pU004pG007pC004pU004pU004pG004p | | |
| | G004 | | |
| 39 | A004pA004pG004pC004pA007pA004pG007pC007pA007pG004p | SS | 1564 |
| | A004pC004pA004pU004pU004pU004pA004pU004pC004 | | |
| | G004pA007pU004pA004pA004pA007pU004pG004pU004pC004p | AS | 1565 |
| | U004pG004pC004pU007pU004pG007pC004pU004pU004pG004p | | |
| | G004 | | |
| 40 | C004pC004pA004pA004pG004pC004pA004pA004pG007pC007p | SS | 1566 |
| | A007pG004pA004pC004pA004pU004pU004pU004pA004pU004p | | |
| | C004 | | |
| | G004pA007pU004pA004pA004pA007pU004pG004pU004pC004p | AS | 1567 |
| | U004pG004pC004pU007pU004pG007pC004pU004pU004pG004p | | |
| | G004pG004pU004 | | |
| 41 | C004pC004pA004pA004pG004pC004pA007pA004pG007pC007p | SS | 1568 |
| | A007pG004pA004pC004pA004pU004pU004pU004pA004pU004p | | |
| | C004 | | |
| | G004pA007pU004pA004pA004pA007pU004pG007pU007pC004p | AS | 1569 |
| | U004pG004pC004pU007pU004pG007pC004pU004pU004pG004p | | |
| | G004pG004pU004 | | |
| 42 | C004pC004pA004pA004pG004pC004pA007pA004pG007pC007p | SS | 1570 |
| | A007pG004pA004pC004pA004pU004pU004pU004pA004pU004p | | |
| | C004 | | |
| | G004pA007pU004pA004pA004pA007pU004pG004pU004pC004p | AS | 1571 |
| | U004pG004pC004pU007pU004pG007pC004pU004pU004pG004p | | |
| | G004pG004pU004 | | |
| 43 | A004p001A004p001G004pC004pA004pA004pG007pC007pA007 | SS | 1572 |
| | pG004pA004pC004pA004pU004pU004pU004pA004pU004pC004 | | |
| | G004p001A007p001U004pA004pA004pA007pU004pG004pU004 | AS | 1573 |
| | pC004pU004pG004pC004pU007pU004pG007pC004pU004pU004 | | |
| | p001G004p001G004 | | |
| 44 | A004p001A004p001G004pC004pA007pA004pG007pC007pA007 | SS | 1574 |
| | pG004pA004pC004pA004pU004pU004pU004pA004pU004pC004 | | |
| | G004p001A007p001U004pA004pA004pA007pU004pG007pU007 | AS | 1575 |
| | pC004pU004pG004pC004pU007pU004pG007pC004pU004pU004 | | |
| | p001G004p001G004 | | |
| 45 | A004p001A004p001G004pC004pA007pA004pG007pC007pA007 | SS | 1576 |
| | pG004pA004pC004pA004pU004pU004pU004pA004pU004pC004 | | |
| | G004p001A007p001U004pA004pA004pA007pU004pG004pU004 | AS | 1577 |
| | pC004pU004pG004pC004pU007pU004pG007pC004pU004pU004 | | |
| | p001G004p001G004 | | |
| 46 | C004p001C004p001A004pA004pG004pC004pA004pA004pG007 | SS | 1578 |
| | pC007pA007pG004pA004pC004pA004pU004pU004pU004pA004 | | |
| | pU004pC004 | | |
| | G004p001A007p001U004pA004pA004pA007pU004pG004pU004 | AS | 1579 |
| | pc004pU004pG004pC004pU007pU004pG007pC004pU004pU004 | | |
| | pG004pG004p001G004p001U004 | | |
| 47 | C004p001C004p001A004pA004pG004pC004pA007pA004pG007 | SS | 1580 |
| | pC007pA007pG004pA004pC004pA004pU004pU004pU004pA004 | | |
| | pU004pC004 | | |
| | G004p001A007p001U004pA004pA004pA007pU004pG007pU007 | AS | 1581 |
| | pC004pU004pG004pC004pU007pU004pG007pC004pU004pU004 | | |
| | pG004pG004p001G004p001U004 | | |
| 48 | C004p001C004p001A004pA004pG004pC004pA007pA004pG007 | SS | 1582 |
| | pC007pA007pG004pA004pC004pA004pU004pU004pU004pA004 | | |
| | pU004pC004 | | |
| | G004p001A007p001U004pA004pA004pA007pU004pG004pU004 | AS | 1583 |
| | pC004pU004pG004pC004pU007pU004pG007pC004pU004pU004 | | |
| | pG004pG004p001G004p001U004 | | |
| 49 | A004pA004pG004pC004pA004pA004pG007pC007pA007pG004p | SS | 1584 |
| | A004pC004pA004pU004pU004pU004pA004pU004pC004 | | |
| | PG004pA007pU004pA004pA004pA007pU004pG004pU004pC004 | AS | 1585 |
| | pU004pG004pC004pU007pU004pG007pC004pU004pU004pG004 | | |
| | pG004 | | |
| 50 | A004pA004pG004pC004pA007pA004pG007pC007pA007pG004p | SS | 1586 |
| | A004pC004pA004pU004pU004pU004pA004pU004pC004 | | |
| | PG004pA007pU004pA004pA004pA007pU004pG007pU007pC004 | AS | 1587 |
| | pU004pG004pC004pU007pU004pG007pC004pU004pU004pG004 | | |
| | pG004 | | |
| 51 | A004pA004pG004pC004pA007pA004pG007pC007pA007pG004p | SS | 1588 |
| | A004pC004pA004pU004pU004pU004pA004pU004pC004 | | |
| | PG004pA007pU004pA004pA004pA007pU004pG004pU004pC004 | AS | 1589 |
| | pU004pG004pC004pU007pU004pG007pC004pU004pU004pG004 | | |
| | pG004 | | |
| 52 | C004pC004pA004pA004pG004pC004pA004pA004pG007pC007p | SS | 1590 |
| | A007pG004pA004pC004pA004pU004pU004pU004pA004pU004p | | |
| | C004 | | |
| | PG004pA007pU004pA004pA004pA007pU004pG004pU004pC004 | AS | 1591 |
| | pU004pG004pC004pU007pU004pG007pC004pU004pU004pG004 | | |
| | pG004pG004pU004 | | |
| 53 | C004pC004pA004pA004pG004pC004pA007pA004pG007pC007p | SS | 1592 |
| | A007pG004pA004pC004pA004pU004pU004pU004pA004pU004p | | |
| | C004 | | |
| | PG004pA007pU004pA004pA004pA007pU004pG007pU007pC004 | AS | 1593 |
| | pU004pG004pC004pU007pU004pG007pC004pU004pU004pG004 | | |
| | pG004pG004pU004 | | |
| 54 | C004pC004pA004pA004pG004pC004pA007pA004pG007pC007p | SS | 1594 |
| | A007pG004pA004pC004pA004pU004pU004pU004pA004pU004p | | |
| | C004 | | |
| | PG004pA007pU004pA004pA004pA007pU004pG004pU004pC004 | AS | 1595 |
| | pU004pG004pC004pU007pU004pG007pC004pU004pU004pG004 | | |
| | pG004pG004pU004 | | |
| 55 | A004p001A004p001G004pC004pA004pA004pG007pC007pA007 | SS | 1596 |
| | pG004pA004pC004pA004pU004pU004pU004pA004pU004pC004 | | |
| | PG004p001A007p001U004pA004pA004pA007pU004pG004pU00 | AS1 | 1597 |
| | 4pC004pU004pG004pC004pU007pU004pG007pC004pU004pU00 | | |
| | 4p001G004p001G004 | | |
| 56 | A004p001A004p001G004pC004pA007pA004pG007pC007pA007 | SS | 1598 |
| | pG004pA004pC004pA004pU004pU004pU004pA004pU004pC004 | | |
| | PG004p001A007p001U004pA004pA004pA007pU004pG007pU00 | AS | 1599 |
| | 7pC004pU004pG004pC004pU007pU004pG007pC004pU004pU00 | | |
| | 4p001G004p001G004 | | |
| 57 | A004p001A004p001G004pC004pA007pA004pG007pC007pA007 | SS | 1600 |
| | pG004pA004pC004pA004pU004pU004pU004pA004pU004pC004 | | |
| | PG004p001A007p001U004pA004pA004pA007pU004pG004pU00 | AS | 1601 |
| | 4pC004pU004pG004pC004pU007pU004pG007pC004pU004pU00 | | |
| | 4p001G004p001G004 | | |
| 58 | C004p001C004p001A004pA004pG004pC004pA004pA004pG007 | SS | 1602 |
| | pC007pA007pG004pA004pC004pA004pU004pU004pU004pA004 | | |
| | pU004pC004 | | |
| | PG004p001A007p001U004pA004pA004pA007pU004pG004pU00 | AS | 1603 |
| | 4pC004pU004pG004pC004pU007pU004pG007pC004pU004pU00 | | |
| | 4pG004pG004p001G004p001U004 | | |
| 59 | C004p001C004p001A004pA004pG004pC004pA007pA004pG007 | SS | 1604 |
| | pC007pA007pG004pA004pC004pA004pU004pU004pU004pA004 | | |
| | pU004pC004 | | |
| | PG004p001A007p001U004pA004pA004pA007pU004pG007pU00 | AS | 1605 |
| | 7pC004pU004pG004pC004pU007pU004pG007pC004pU004pU00 | | |
| | 4pG004pG004p001G004p001U004 | | |
| 60 | C004p001C004p001A004pA004pG004pC004pA007pA004pG007 | SS | 1606 |
| | pC007pA007pG004pA004pC004pA004pU004pU004pU004pA004 | | |
| | pU004pC004 | | |
| | PG004p001A007p001U004pA004pA004pA007pU004pG004pU00 | AS | 1607 |
| | 4pC004pU004pG004pC004pU007pU004pG007pC004pU004pU00 | | |
| | 4pG004pG004p001G004p001U004 | | |
| 61 | U000pU000pU000pG000pU000pA000pG000pC000pA000pU000p | SS | 1608 |
| | U000pU000pU000pU000pA000pU000pU000pA000pA000 | | |
| | U000pU000pA000pA000pU000pA000pA000pA000pA000pA000p | AS | 1609 |
| | U000pG000pC000pU000pA000pC000pA000pA000pA000pA000p | | |
| | C000 | | |
| 62 | G000pU000pU000pU000pU000pG000pU000pA000pG000pC000p | SS | 1610 |
| | A000pU000pU000pU000pU000pU000pA000pU000pU000pA000p | | |
| | A000 | | |
| | U000pU000pA000pA000pU000pA000pA000pA000pA000pA000p | AS | 1611 |
| | U000pG000pC000pU000pA000pC000pA000pA000pA000pA000p | | |
| | C000pC000pC000 | | |
| 63 | U004pU004pU004pG004pU004pA004pG007pC007pA007pU004p | SS | 1612 |
| | U004pU004pU004pU004pA004pU004pU004pA004pA004 | | |
| | U004pU007pA004pA004pU004pA007pA004pA004pA004pA004p | AS | 1613 |
| | U004pG007pC004pU007pA004pC004pA004pA004pA004pA004p | | |
| | C004 | | |
| 64 | U004pU004pU004pG004pU007pA004pG007pC007pA007pU004p | SS | 1614 |
| | U004pU004pU004pU004pA004pU004pU004pA004pA004 | | |
| | U004pU007pA004pA004pU004pA007pA004pA007pA007pA004p | AS | 1615 |
| | U004pG007pC004pU007pA004pC004pA004pA004pA004pA004p | | |
| | C004 | | |
| 65 | U004pU004pU004pG004pU007pA004pG007pC007pA007pU004p | SS | 1616 |
| | U004pU004pU004pU004pA004pU004pU004pA004pA004 | | |
| | U004pU007pA004pA004pU004pA007pA004pA004pA004pA004p | AS | 1617 |
| | U004pG007pC004pU007pA004pC004pA004pA004pA004pA004p | | |
| | C004 | | |
| 66 | G004pU004pU004pU004pU004pG004pU004pA004pG007pC007p | SS | 1618 |
| | A007pU004pU004pU004pU004pU004pA004pU004pU004pA004p | | |
| | A004 | | |
| | U004pU007pA004pA004pU004pA007pA004pA004pA004pA004p | AS | 1619 |
| | U004pG007pC004pU007pA004pC004pA004pA004pA004pA004p | | |
| | C004pC004pC004 | | |
| 67 | G004pU004pU004pU004pU004pG004pU007pA004pG007pC007p | SS | 1620 |
| | A007pU004pU004pU004pU004pU004pA004pU004pU004pA004p | | |
| | A004 | | |
| | U004pU007pA004pA004pU004pA007pA004pA007pA007pA004p | AS | 1621 |
| | U004pG007pC004pU007pA004pC004pA004pA004pA004pA004p | | |
| | C004pC004pC004 | | |
| 68 | G004pU004pU004pU004pU004pG004pU007pA004pG007pC007p | SS | 1622 |
| | A007pU004pU004pU004pU004pU004pA004pU004pU004pA004p | | |
| | A004 | | |
| | U004pU007pA004pA004pU004pA007pA004pA004pA004pA004p | AS | 1623 |
| | U004pG007pC004pU007pA004pC004pA004pA004pA004pA004p | | |
| | C004pC004pC004 | | |
| 69 | U004p001U004p001U004pG004pU004pA004pG007pC007pA007 | SS | 1624 |
| | pU004pU004pU004pU004pU004pA004pU004pU004pA004pA004 | | |
| | U004p001U007p001A004pA004pU004pA007pA004pA004pA004 | AS | 1625 |
| | pA004pU004pG007pC004pU007pA004pC004pA004pA004pA004 | | |
| | p001A004p001C004 | | |
| 70 | U004p001U004p001U004pG004pU007pA004pG007pC007pA007 | SS | 1626 |
| | pU004pU004pU004pU004pU004pA004pU004pU004pA004pA004 | | |
| | U004p001U007p001A004pA004pU004pA007pA004pA007pA007 | AS | 1627 |
| | pA004pU004pG007pC004pU007pA004pC004pA004pA004pA004 | | |
| | p001A004p001C004 | | |
| 71 | U004p001U004p001U004pG004pU007pA004pG007pC007pA007 | SS | 1628 |
| | pU004pU004pU004pU004pU004pA004pU004pU004pA004pA004 | | |
| | U004p001U007p001A004pA004pU004pA007pA004pA004pA004 | AS | 1629 |
| | pA004pU004pG007pC004pU007pA004pC004pA004pA004pA004 | | |
| | p001A004p001C004 | | |
| 72 | G004p001U004p001U004pU004pU004pG004pU004pA004pG007 | SS | 1630 |
| | pC007pA007pU004pU004pU004pU004pU004pA004pU004pU004 | | |
| | pA004pA004 | | |
| | U004p001U007p001A004pA004pU004pA007pA004pA004pA004 | AS | 1631 |
| | pA004pU004pG007pC004pU007pA004pC004pA004pA004pA004 | | |
| | pA004pC004p001C004p001C004 | | |
| 73 | G004p001U004p001U004pU004pU004pG004pU007pA004pG007 | SS | 1632 |
| | pC007pA007pU004pU004pU004pU004pU004pA004pU004pU004 | | |
| | pA004pA004 | | |
| | U004p001U007p001A004pA004pU004pA007pA004pA007pA007 | AS | 1633 |
| | pA004pU004pG007pC004pU007pA004pC004pA004pA004pA004 | | |
| | pA004pC004p001C004p001C004 | | |
| 74 | G004p001U004p001U004pU004pU004pG004pU007pA004pG007 | SS | 1634 |
| | pC007pA007pU004pU004pU004pU004pU004pA004pU004pU004 | | |
| | pA004pA004 | | |
| | U004p001U007p001A004pA004pU004pA007pA004pA004pA004 | AS | 1635 |
| | pA004pU004pG007pC004pU007pA004pC004pA004pA004pA004 | | |
| | pA004pC004p001C004p001C004 | | |
| 75 | U004pU004pU004pG004pU004pA004pG007pC007pA007pU004p | SS | 1636 |
| | U004pU004pU004pU004pA004pU004pU004pA004pA004 | | |
| | PU004pU007pA004pA004pU004pA007pA004pA004pA004pA004 | AS | 1637 |
| | pU004pG007pC004pU007pA004pC004pA004pA004pA004pA004 | | |
| | pC004 | | |
| 76 | U004pU004pU004pG004pU007pA004pG007pC007pA007pU004p | SS | 1638 |
| | U004pU004pU004pU004pA004pU004pU004pA004pA004 | | |
| | PU004pU007pA004pA004pU004pA007pA004pA007pA007pA004 | AS | 1639 |
| | pU004pG007pC004pU007pA004pC004pA004pA004pA004pA004 | | |
| | pC004 | | |
| 77 | U004pU004pU004pG004pU007pA004pG007pC007pA007pU004p | SS | 1640 |
| | U004pU004pU004pU004pA004pU004pU004pA004pA004p | | |
| | PU004pU007pA004pA004pU004pA007pA004pA004pA004pA004 | AS | 1641 |
| | pU004pG007pC004pU007pA004pC004pA004pA004pA004pA004 | | |
| | pC004 | | |
| 78 | G004pU004pU004pU004pU004pG004pU004pA004pG007pC007p | SS | 1642 |
| | A007pU004pU004pU004pU004pU004pA004pU004pU004pA004p | | |
| | A004 | | |
| | PU004pU007pA004pA004pU004pA007pA004pA004pA004pA004 | AS | 1643 |
| | pU004pG007pC004pU007pA004pC004pA004pA004pA004pA004 | | |
| | pC004pC004pC004 | | |
| 79 | G004pU004pU004pU004pU004pG004pU007pA004pG007pC007p | SS | 1644 |
| | A007pU004pU004pU004pU004pU004pA004pU004pU004pA004p | | |
| | A004 | | |
| | PU004pU007pA004pA004pU004pA007pA004pA007pA007pA004 | AS | 1645 |
| | pU004pG007pC004pU007pA004pC004pA004pA004pA004pA004 | | |
| | pc004pC004pC004 | | |
| 80 | G004pU004pU004pU004pU004pG004pU007pA004pG007pC007p | SS | 1646 |
| | A007pU004pU004pU004pU004pU004pA004pU004pU004pA004p | | |
| | A004 | | |
| | PU004pU007pA004pA004pU004pA007pA004pA004pA004pA004 | AS | 1647 |
| | pU004pG007pC004pU007pA004pC004pA004pA004pA004pA004 | | |
| | pC004pC004pC004 | | |
| 81 | U004p001U004p001U004pG004pU004pA004pG007pC007pA007 | SS | 1648 |
| | pU004pU004pU004pU004pU004pA004pU004pU004pA004pA004 | | |
| | PU004p001U007p001A004pA004pU004pA007pA004pA004pA00 | AS | 1649 |
| | 4pA004pU004pG007pC004pU007pA004pC004pA004pA004pA00 | | |
| | 4p001A004p001C004 | | |
| 82 | U004p001U004p001U004pG004pU007pA004pG007pC007pA007 | SS | 1650 |
| | pU004pU004pU004pU004pU004pA004pU004pU004pA004pA004 | | |
| | PU004p001U007p001A004pA004pU004pA007pA004pA007pA00 | AS | 1651 |
| | 7pA004pU004pG007pC004pU007pA004pC004pA004pA004pA00 | | |
| | 4p001A004p001C004 | | |
| 83 | U004p001U004p001U004pG004pU007pA004pG007pC007pA007 | SS | 1652 |
| | pU004pU004pU004pU004pU004pA004pU004pU004pA004pA004 | | |
| | PU004p001U007p001A004pA004pU004pA007pA004pA004pA00 | AS | 1653 |
| | 4pA004pU004pG007pC004pU007pA004pC004pA004pA004pA00 | | |
| | 4p001A004p001C004 | | |
| 84 | G004p001U004p001U004pU004pU004pG004pU004pA004pG007 | SS | 1654 |
| | pC007pA007pU004pU004pU004pU004pU004pA004pU004pU004 | | |
| | pA004pA004 | | |
| | PU004p001U007p001A004pA004pU004pA007pA004pA004pA00 | AS | 1655 |
| | 4pA004pU004pG007pC004pU007pA004pC004pA004pA004pA00 | | |
| | 4pA004pC004p001C004p001C004 | | |
| 85 | G004p001U004p001U004pU004pU004pG004pU007pA004pG007 | SS | 1656 |
| | pc007pA007pU004pU004pU004pU004pU004pA004pU004pU004 | | |
| | pA004pA004 | | |
| | pU004p001U007p001A004pA004pU004pA007pA004pA007pA00 | AS | 1657 |
| | 7pA004pU004pG007pC004pU007pA004pC004pA004pA004pA00 | | |
| | 4pA004pC004p001C004p001C004 | | |
| 86 | G000pC000pC000pU000pG000pG000pA000pG000pU000pU000p | SS | 1658 |
| | U000pA000pU000pU000pC000pG000pG000pA000pA000 | | |
| | U000pU000pC000pC000pG000pA000pA000pU000pA000pA000p | AS | 1659 |
| | A000pC000pU000pC000pC000pA000pG000pG000pC000pC000p | | |
| | U000 | | |
| 87 | A000pG000pG000pC000pC000pU000pG000pG000pA000pG000p | SS | 1660 |
| | U000pU000pU000pA000pU000pU000pC000pG000pG000pA000p | | |
| | A000 | | |
| | U000pU000pC000pC000pG000pA000pA000pU000pA000pA000p | AS | 1661 |
| | A000pC000pU000pC000pC000pA000pG000pG000pC000pC000p | | |
| | U000pA000pU000 | | |
| 88 | G004pC004pC004pU004pG004pG004pA007pG007pU007pU004p | SS | 1662 |
| | U004pA004pU004pU004pC004pG004pG004pA004pA004 | | |
| | U004pU007pC004pC004pG004pA007pA004pU004pA004pA004p | AS | 1663 |
| | A004pC004pU004pC007pC004pA007pG004pG004pC004pC004p | | |
| | U004 | | |
| 89 | G004pC004pC004pU004pG007pG004pA007pG007pU007pU004p | SS | 1664 |
| | U004pA004pU004pU004pC004pG004pG004pA004pA004 | | |
| | U004pU007pC004pC004pG004pA007pA004pU007pA007pA004p | AS | 1665 |
| | A004pC004pU004pC007pC004pA007pG004pG004pC004pC004p | | |
| | U004 | | |
| 90 | G004pC004pC004pU004pG007pG004pA007pG007pU007pU004p | SS | 1666 |
| | U004pA004pU004pU004pC004pG004pG004pA004pA004 | | |
| | U004pU007pC004pC004pG004pA007pA004pU004pA004pA004p | AS | 1667 |
| | A004pC004pU004pC007pC004pA007pG004pG004pC004pC004p | | |
| | U004 | | |
| 91 | A004pG004pG004pC004pC004pU004pG004pG004pA007pG007p | SS | 1668 |
| | U007pU004pU004pA004pU004pU004pC004pG004pG004pA004p | | |
| | A004 | | |
| | U004pU007pC004pC004pG004pA007pA004pU004pA004pA004p | AS | 1669 |
| | A004pC004pU004pC007pC004pA007pG004pG004pC004pC004p | | |
| | U004pA004pU004 | | |
| 92 | A004pG004pG004pC004pC004pU004pG007pG004pA007pG007p | SS | 1670 |
| | U007pU004pU004pA004pU004pU004pC004pG004pG004pA004p | | |
| | A004 | | |
| | U004pU007pC004pC004pG004pA007pA004pU007pA007pA004p | AS | 1671 |
| | A004pC004pU004pC007pC004pA007pG004pG004pC004pC004p | | |
| | U004pA004pU004 | | |
| 93 | A004pG004pG004pC004pC004pU004pG007pG004pA007pG007p | SS | 1672 |
| | U007pU004pU004pA004pU004pU004pC004pG004pG004pA004p | | |
| | A004 | | |
| | U004pU007pC004pC004pG004pA007pA004pU004pA004pA004p | AS | 1673 |
| | A004pC004pU004pC007pC004pA007pG004pG004pC004pC004p | | |
| | U004pA004pU004 | | |
| 94 | G004p001C004p001C004pU004pG004pG004pA007pG007pU007 | SS | 1674 |
| | pU004pU004pA004pU004pU004pC004pG004pG004pA004pA004 | | |
| | U004p001U007p001C004pC004pG004pA007pA004pU004pA004 | AS | 1675 |
| | pA004pA004pC004pU004pC007pC004pA007pG004pG004pC004 | | |
| | p001C004p001U004 | | |
| 95 | G004p001C004p001C004pU004pG007pG004pA007pG007pU007 | SS | 1676 |
| | pU004pU004pA004pU004pU004pC004pG004pG004pA004pA004 | | |
| | U004p001U007p001C004pC004pG004pA007pA004pU007pA007 | AS | 1677 |
| | pA004pA004pC004pU004pC007pC004pA007pG004pG004pC004 | | |
| | p001C004p001U004 | | |
| 96 | G004p001C004p001C004pU004pG007pG004pA007pG007pU007 | SS | 1678 |
| | pU004pU004pA004pU004pU004pC004pG004pG004pA004pA004 | | |
| | U004p001U007p001C004pC004pG004pA007pA004pU004pA004 | AS | 1679 |
| | pA004pA004pC004pU004pC007pC004pA007pG004pG004pC004 | | |
| | p001C004p0010004 | | |
| 97 | A004p001G004p001G004pC004pC004pU004pG004pG004pA007 | SS | 1680 |
| | pG007pU007pU004pU004pA004pU004pU004pC004pG004pG004 | | |
| | pA004pA004 | | |
| | U004p001U007p001C004pC004pG004pA007pA004pU004pA004 | AS | 1681 |
| | pA004pA004pC004pU004pC007pC004pA007pG004pG004pC004 | | |
| | pC004pU004p001A004p001U004 | | |
| 98 | A004p001G004p001G004pC004pC004pU004pG007pG004pA007 | SS | 1682 |
| | pG007pU007pU004pU004pA004pU004pU004pC004pG004pG004 | | |
| | pA004pA004 | | |
| | U004p001U007p001C004pC004pG004pA007pA004pU007pA007 | AS | 1683 |
| | pA004pA004pC004pU004pC007pC004pA007pG004pG004pC004 | | |
| | pC004pU004p001A004p001U004 | | |
| 99 | A004p001G004p001G004pC004pC004pU004pG007pG004pA007 | SS | 1684 |
| | pG007pU007pU004pU004pA004pU004pU004pC004pG004pG004 | | |
| | pA004pA004 | | |
| | U004p001U007p001C004pC004pG004pA007pA004pU004pA004 | AS | 1685 |
| | pA004pA004pC004pU004pC007pC004pA007pG004pG004pC004 | | |
| | pC004pU004p001A004p001U004 | | |
| 100 | G004pC004pC004pU004pG004pG004pA007pG007pU007pU004p | SS | 1686 |
| | U004pA004pU004pU004pC004pG004pG004pA004pA004 | | |
| | PU004pU007pC004pC004pG004pA007pA004pU004pA004pA004 | AS | 1687 |
| | pA004pC004pU004pC007pC004pA007pG004pG004pC004pC004 | | |
| | pU004 | | |
| 101 | G004pC004pC004pU004pG007pG004pA007pG007pU007pU004p | SS | 1688 |
| | U004pA004pU004pU004pC004pG004pG004pA004pA004 | | |
| | PU004pU007pC004pC004pG004pA007pA004pU007pA007pA004 | AS | 1689 |
| | pA004pC004pU004pC007pC004pA007pG004pG004pC004pC004 | | |
| | pU004 | | |
| 102 | G004pC004pC004pU004pG007pG004pA007pG007pU007pU004p | SS | 1690 |
| | U004pA004pU004pU004pC004pG004pG004pA004pA004 | | |
| | PU004pU007pC004pC004pG004pA007pA004pU004pA004pA004 | AS | 1691 |
| | pA004pC004pU004pC007pC004pA007pG004pG004pC004pC004 | | |
| | pU004 | | |
| 103 | A004pG004pG004pC004pC004pU004pG004pG004pA007pG007p | SS | 1692 |
| | U007pU004pU004pA004pU004pU004pC004pG004pG004pA004p | | |
| | A004 | | |
| | PU004pU007pC004pC004pG004pA007pA004pU004pA004pA004 | AS | 1693 |
| | pA004pC004pU004pC007pC004pA007pG004pG004pC004pC004 | | |
| | pU004pA004pU004 | | |
| 104 | A004pG004pG004pC004pC004pU004pG007pG004pA007pG007p | SS | 1694 |
| | U007pU004pU004pA004pU004pU004pC004pG004pG004pA004p | | |
| | A004 | | |
| | PU004pU007pC004pC004pG004pA007pA004pU007pA007pA004 | AS | 1695 |
| | pA004pC004pU004pC007pC004pA007pG004pG004pC004pC004 | | |
| | pU004pA004pU004 | | |
| 105 | A004pG004pG004pC004pC004pU004pG007pG004pA007pG007p | SS | 1696 |
| | U007pU004pU004pA004pU004pU004pC004pG004pG004pA004p | | |
| | A004 | | |
| | PU004pU007pC004pC004pG004pA007pA004pU004pA004pA004 | AS | 1697 |
| | pA004pC004pU004pC007pC004pA007pG004pG004pC004pC004 | | |
| | pU004pA004pU004 | | |
| 106 | G004p001C004p001C004pU004pG004pG004pA007pG007pU007 | SS | 1698 |
| | pU004pU004pA004pU004pU004pC004pG004pG004pA004pA004 | | |
| | PU004p001U007p001C004pC004pG004pA007pA004pU004pA00 | AS | 1699 |
| | 4pA004pA004pC004pU004pC007pC004pA007pG004pG004pC00 | | |
| | 4p001C004p001U004 | | |
| 107 | G004p001C004p001C004pU004pG007pG004pA007pG007pU007 | SS | 1700 |
| | pU004pU004pA004pU004pU004pC004pG004pG004pA004pA004 | | |
| | pU004p001U007p001C004pC004pG004pA007pA004pU007pA00 | AS | 1701 |
| | 7pA004pA004pC004pU004pC007pC004pA007pG004pG004pC00 | | |
| | 4p001C004p001U004 | | |
| 107 | G004p001C004p001C004pU004pG007pG004pA007pG007pU007 | SS | 1702 |
| | pU004pU004pA004pU004pU004pC004pG004pG004pA004pA004 | | |
| | pU004p001U007p001C004pC004pG004pA007pA004pU004pA00 | AS | 1703 |
| | 4pA004pA004pC004pU004pC007pC004pA007pG004pG004pC00 | | |
| | 4p001C004p001U004 | | |
| 109 | A004p001G004p001G004pC004pC004pU004pG004pG004pA007 | SS | 1704 |
| | pG007pU007pU004pU004pA004pU004pU004pC004pG004pG004 | | |
| | pA004pA004 | | |
| | pU004p001U007p001C004pC004pG004pA007pA004pU004pA00 | AS | 1705 |
| | 4pA004pA004pC004pU004pC007pC004pA007pG004pG004pC00 | | |
| | 4pC004pU004p001A004p001U004 | | |
| 110 | A004p001G004p001G004pC004pC004pU004pG007pG004pA007 | SS | 1706 |
| | pG007pU007pU004pU004pA004pU004pU004pC004pG004pG004 | | |
| | pA004pA004 | | |
| | PU004p001U007p001C004pC004pG004pA007pA004pU007pA00 | AS | 1707 |
| | 7pA004pA004pC004pU004pC007pC004pA007pG004pG004pC00 | | |
| | 4pC004pU004p001A004p001U004 | | |
| 111 | A004p001G004p001G004pC004pC004pU004pG007pG004pA007 | SS | 1708 |
| | pG007pU007pU004pU004pA004pU004pU004pC004pG004pG004 | | |
| | pA004pA004 | | |
| | pU004p001U007p001C004pC004pG004pA007pA004pU004pA00 | AS | 1709 |
| | 4pA004pA004pC004pU004pC007pC004pA007pG004pG004pC00 | | |
| | 4pC004pU004p001A004p001U004 | | |
| 112 | C000000G000pU000pU000pU000pU000pG000pC000pU000p | SS | 1710 |
| | U000pU000pU000pG000pU000pA000pA000pC000pU000 | | |
| | A000pG000pU000pU000pA000pC000pA000pA000pA000pA000p | AS | 1711 |
| | G000pC000pA000pA000pA000pA000pC000pA000pG000pG000p | | |
| | U000 | | |
| 113 | A000000000000pG000pU000pU000pU000pU000pG000p | SS | 1712 |
| | C000pU000pU000pU000pU000pG000pU000pA000pA000pC000p | | |
| | U000 | | |
| | A000pG000pU000pU000pA000pC000pA000pA000pA000pA000p | AS | 1713 |
| | G000pC000pA000pA000pA000pA000pC000pA000pG000pG000p | | |
| | U000pC000pU000 | | |
| 114 | C004pU004pG004pU004pU004pU004pU007pG007pC007pU004p | SS | 1714 |
| | U004pU004pU004pG004pU004pA004pA004pC004pU004p | | |
| | A004pG007pU004pU004pA004pC007pA004pA004pA004pA004p | AS | 1715 |
| | G004pC004pA004pA007pA004pA007pC004pA004pG004pG004p | | |
| | U004 | | |
| 115 | C004pU004pG004pU004pU007pU004pU007pG007pC007pU004p | SS | 1716 |
| | U004pU004pU004pG004pU004pA004pA004pC004pU004 | | |
| | A004pG007pU004pU004pA004pC007pA004pA007pA007pA004p | AS | 1717 |
| | G004pC004pA004pA007pA004pA007pC004pA004pG004pG004p | | |
| | U004 | | |
| 116 | C004pU004pG004pU004pU007pU004pU007pG007pC007pU004p | SS | 1718 |
| | U004pU004pU004pG004pU004pA004pA004pC004pU004 | | |
| | A004pG007pU004pU004pA004pC007pA004pA004pA004pA004p | AS | 1719 |
| | G004pC004pA004pA007pA004pA007pC004pA004pG004pG004p | | |
| | U004 | | |
| 117 | A004pC004pC004pU004pG004pU004pU004pU004pU007pG007p | SS | 1720 |
| | C007pU004pU004pU004pU004pG004pU004pA004pA004pC004p | | |
| | U004 | | |
| | A004pG007pU004pU004pA004pC007pA004pA004pA004pA004p | AS | 1721 |
| | G004pC004pA004pA007pA004pA007pC004pA004pG004pG004p | | |
| | U004pC004pU004 | | |
| 118 | A004pC004pC004pU004pG004pU004pU007pU004pU007pG007p | SS | 1722 |
| | C007pU004pU004pU004pU004pG004pU004pA004pA004pC004p | | |
| | U004 | | |
| | A004pG007pU004pU004pA004pC007pA004pA007pA007pA004p | AS | 1723 |
| | G004pC004pA004pA007pA004pA007pC004pA004pG004pG004p | | |
| | U004pC004pU004 | | |
| 119 | A004pC004pC004pU004pG004pU004pU007pU004pU007pG007p | SS | 1724 |
| | C007pU004pU004pU004pU004pG004pU004pA004pA004pC004p | | |
| | U004 | | |
| | A004pG007pU004pU004pA004pC007pA004pA004pA004pA004p | AS | 1725 |
| | G004pC004pA004pA007pA004pA007pC004pA004pG004pG004p | | |
| | U004pC004pU004 | | |
| 120 | C004p001U004p001G004pU004pU004pU004pU007pG007pC007 | SS | 1726 |
| | pU004pU004pU004pU004pG004pU004pA004pA004pC004pU004 | | |
| | A004p001G007p001U004pU004pA004pC007pA004pA004pA004 | AS | 1727 |
| | pA004pG004pC004pA004pA007pA004pA007pC004pA004pG004 | | |
| | p001G004p001U004 | | |
| 121 | C004p001U004p001G004pU004pU007pU004pU007pG007pC007 | SS | 1728 |
| | pU004pU004pU004pU004pG004pU004pA004pA004pC004pU004 | | |
| | A004p001G007p001U004pU004pA004pC007pA004pA007pA007 | AS | 1729 |
| | pA004pG004pC004pA004pA007pA004pA007pC004pA004pG004 | | |
| | p001G004p001U004 | | |
| 122 | C004p001U004p001G004pU004pU007pU004pU007pG007pC007 | SS | 1730 |
| | pU004pU004pU004pU004pG004pU004pA004pA004pC004pU004 | | |
| | A004p001G007p001U004pU004pA004pC007pA004pA004pA004 | AS | 1731 |
| | pA004pG004pC004pA004pA007pA004pA007pC004pA004pG004 | | |
| | p001G004p001U004 | | |
| 123 | A004p001C004p001C004pU004pG004pU004pU004pU004pU007 | SS | 1732 |
| | pG007pC007pU004pU004pU004pU004pG004pU004pA004pA004 | | |
| | pC004pU004 | | |
| | A004p001G007p001U004pU004pA004pC007pA004pA004pA004 | AS | 1733 |
| | pA004pG004pC004pA004pA007pA004pA007pC004pA004pG004 | | |
| | pG004pU004p001C004p001U004 | | |
| 124 | A004p001C004p001C004pU004pG004pU004pU007pU004pU007 | SS | 1734 |
| | pG007pC007pU004pU004pU004pU004pG004pU004pA004pA004 | | |
| | pC004pU004 | | |
| | A004p001G007p001U004pU004pA004pC007pA004pA007pA007 | AS | 1735 |
| | pA004pG004pC004pA004pA007pA004pA007pC004pA004pG004 | | |
| | pG004pU004p001C004p001U004 | | |
| 125 | A004p001C004p001C004pU004pG004pU004pU007pU004pU007 | SS | 1736 |
| | pG007pC007pU004pU004pU004pU004pG004pU004pA004pA004 | | |
| | pC004pU004 | | |
| | A004p001G007p001U004pU004pA004pC007pA004pA004pA004 | AS | 1737 |
| | pA004pG004pC004pA004pA007pA004pA007pC004pA004pG004 | | |
| | pG004pU004p001C004p001U004 | | |
| 126 | C004pU004pG004pU004pU004pU004pU007pG007pC007pU004p | SS | 1738 |
| | U004pU004pU004pG004pU004pA004pA004pC004pU004 | | |
| | pA004pG007pU004pU004pA004pC007pA004pA004pA004pA004 | AS | 1739 |
| | pG004pC004pA004pA007pA004pA007pC004pA004pG004pG004 | | |
| | pU004 | | |
| 127 | C004pU004pG004pU004pU007pU004pU007pG007pC007pU004p | SS | 1740 |
| | U004pU004pU004pG004pU004pA004pA004pC004pU004 | | |
| | pA004pG007pU004pU004pA004pC007pA004pA007pA007pA004 | AS | 1741 |
| | pG004pC004pA004pA007pA004pA007pC004pA004pG004pG004 | | |
| | pU004 | | |
| 128 | C004pU004pG004pU004pU007pU004pU007pG007pC007pU004p | SS | 1742 |
| | U004pU004pU004pG004pU004pA004pA004pC004pU004 | | |
| | PA004pG007pU004pU004pA004pC007pA004pA004pA004pA004 | AS | 1743 |
| | pG004pC004pA004pA007pA004pA007pC004pA004pG004pG004 | | |
| | pU004 | | |
| 129 | A004pC004pC004pU004pG004pU004pU004pU004pU007pG007p | SS | 1744 |
| | C007pU004pU004pU004pU004pG004pU004pA004pA004pC004p | | |
| | U004 | | |
| | pA004pG007pU004pU004pA004pC007pA004pA004pA004pA004 | AS | 1745 |
| | pG004pC004pA004pA007pA004pA007pC004pA004pG004pG004 | | |
| | pU004pC004pU004 | | |
| 130 | A004pC004pC004pU004pG004pU004pU007pU004pU007pG007p | SS | 1746 |
| | C007pU004pU004pU004pU004pG004pU004pA004pA004pC004p | | |
| | U004 | | |
| | pA004pG007pU004pU004pA004pC007pA004pA007pA007pA004 | AS | 1747 |
| | pG004pC004pA004pA007pA004pA007pC004pA004pG004pG004 | | |
| | pU004pC004pU004 | | |
| 131 | A004pC004pC004pU004pG004pU004pU007pU004pU007pG007p | SS | 1748 |
| | C007pU004pU004pU004pU004pG004pU004pA004pA004pC004p | | |
| | U004 | | |
| | pA004pG007pU004pU004pA004pC007pA004pA004pA004pA004 | AS | 1749 |
| | pG004pC004pA004pA007pA004pA007pC004pA004pG004pG004 | | |
| | pU004pC004pU004 | | |
| 132 | C004p001U004p001G004pU004pU004pU004pU007pG007pC007 | SS | 1750 |
| | pU004pU004pU004pU004pG004pU004pA004pA004pC004pU004 | | |
| | pA004p001G007p001U004pU004pA004pC007pA004pA004pA00 | AS | 1751 |
| | 4pA004pG004pC004pA004pA007pA004pA007pC004pA004pG00 | | |
| | 4p001G004p001U004 | | |
| 133 | C004p001U004p001G004pU004pU007pU004pU007pG007pC007 | SS | 1752 |
| | pU004pU004pU004pU004pG004pU004pA004pA004pC004pU004 | | |
| | pA004p001G007p001U004pU004pA004pC007pA004pA007pA00 | AS | 1753 |
| | 7pA004pG004pC004pA004pA007pA004pA007pC004pA004pG00 | | |
| | 4p001G004p001U004 | | |
| 134 | C004p001U004p001G004pU004pU007pU004pU007pG007pC007 | SS | 1754 |
| | pU004pU004pU004pU004pG004pU004pA004pA004pC004pU004 | | |
| | pA004p001G007p001U004pU004pA004pC007pA004pA004pA00 | AS | 1755 |
| | 4pA004pG004pC004pA004pA007pA004pA007pC004pA004pG00 | | |
| | 4p001G004p001U004 | | |
| 135 | A004p001C004p001C004pU004pG004pU004pU004pU004pU007 | SS | 1756 |
| | pG007pC007pU004pU004pU004pU004pG004pU004pA004pA004 | | |
| | pC004pU004 | | |
| | pA004p001G007p001U004pU004pA004pC007pA004pA004pA00 | AS | 1757 |
| | 4pA004pG004pC004pA004pA007pA004pA007pC004pA004pG00 | | |
| | 4pG004pU004p001C004p001U004 | | |
| 136 | A004p001C004p001C004pU004pG004pU004pU007pU004pU007 | SS | 1758 |
| | pG007pC007pU004pU004pU004pU004pG004pU004pA004pA004 | | |
| | pC004pU004 | | |
| | pA004p001G007p001U004pU004pA004pC007pA004pA007pA00 | AS | 1759 |
| | 7pA004pG004pC004pA004pA007pA004pA007pC004pA004pG00 | | |
| | 4pG004pU004p001C004p001U004 | | |
| 137 | A004p001C004p001C004pU004pG004pU004pU007pU004pU007 | SS | 1760 |
| | pG007pC007pU004pU004pU004pU004pG004pU004pA004pA004 | | |
| | pC004pU004 | | |
| | pA004p001G007p001U004pU004pA004pC007pA004pA004pA00 | AS | 1761 |
| | 4pA004pG004pC004pA004pA007pA004pA007pC004pA004pG00 | | |
| | 4pG004pU004p001C004p001U004 | | |
| 138 | G000pG000pU000pU000pU000pU000pG000pU000pA000pG000p | SS | 1762 |
| | C000pA000pU000pU000pU000pU000pU000pA000pU000 | | |
| | A000pU000pA000pA000pA000pA000pA000pU000pG000pC000p | AS | 1763 |
| | U000pA000pC000pA000pA000pA000pA000pC000pC000pC000p | | |
| | A000 | | |
| 139 | U000pG000pG000pG000pU000pU000pU000pU000pG000pU000p | SS | 1764 |
| | A000pG000pC000pA000pU000pU000pU000pU000pU000pA000p | | |
| | U000 | | |
| | A000pU000pA000pA000pA000pA000pA000pU000pG000pC000p | AS | 1765 |
| | U000pA000pC000pA000pA000pA000pA000pC000pC000pC000p | | |
| | A000pG000pA000 | | |
| 140 | G004pG004pU004pU004pU004pU004pG007pU007pA007pG004p | SS | 1766 |
| | C004pA004pU004pU004pU004pU004pU004pA004pU004 | | |
| | A004pU007pA004pA004pA004pA007pA004pU004pG004pC004p | AS | 1767 |
| | U004pA004pC004pA007pA004pA007pA004pC004pC004pC004p | | |
| | A004 | | |
| 141 | G004pG004pU004pU004pU007pU004pG007pU007pA007pG004p | SS | 1768 |
| | C004pA004pU004pU004pU004pU004pU004pA004pU004 | | |
| | A004pU007pA004pA004pA004pA007pA004pU007pG007pC004p | AS | 1769 |
| | U004pA004pC004pA007pA004pA007pA004pC004pC004pC004p | | |
| | A004 | | |
| 142 | G004pG004pU004pU004pU007pU004pG007pU007pA007pG004p | SS | 1770 |
| | C004pA004pU004pU004pU004pU004pU004pA004pU004 | | |
| | A004pU007pA004pA004pA004pA007pA004pU004pG004pC004p | AS | 1771 |
| | U004pA004pC004pA007pA004pA007pA004pC004pC004pC004p | | |
| | A004 | | |
| 143 | U004pG004pG004pG004pU004pU004pU004pU004pG007pU007p | SS | 1772 |
| | A007pG004pC004pA004pU004pU004pU004pU004pU004pA004p | | |
| | U004 | | |
| | A004pU007pA004pA004pA004pA007pA004pU004pG004pC004p | AS | 1773 |
| | U004pA004pC004pA007pA004pA007pA004pC004pC004pC004p | | |
| | A004pG004pA004 | | |
| 144 | U004pG004pG004pG004pU004pU004pU007pU004pG007pU007p | SS | 1774 |
| | A007pG004pC004pA004pU004pU004pU004pU004pU004pA004p | | |
| | U004 | | |
| | A004pU007pA004pA004pA004pA007pA004pU007pG007pC004p | AS | 1775 |
| | U004pA004pC004pA007pA004pA007pA004pC004pC004pC004p | | |
| | A004pG004pA004 | | |
| 145 | U004pG004pG004pG004pU004pU004pU007pU004pG007pU007p | SS | 1776 |
| | A007pG004pC004pA004pU004pU004pU004pU004pU004pA004p | | |
| | U004 | | |
| | A004pU007pA004pA004pA004pA007pA004pU004pG004pC004p | AS | 1777 |
| | U004pA004pC004pA007pA004pA007pA004pC004pC004pC004p | | |
| | A004pG004pA004 | | |
| 146 | G004p001G004p001U004pU004pU004pU004pG007pU007pA007 | SS | 1778 |
| | pG004pC004pA004pU004pU004pU004pU004pU004pA004pU004 | | |
| | A004p001U007p001A004pA004pA004pA007pA004pU004pG004 | AS | 1779 |
| | pC004pU004pA004pC004pA007pA004pA007pA004pC004pC004 | | |
| | p001C004p001A004 | | |
| 147 | G004p001G004p001U004pU004pU007pU004pG007pU007pA007 | SS | 1780 |
| | pG004pC004pA004pU004pU004pU004pU004pU004pA004pU004 | | |
| | A004p001U007p001A004pA004pA004pA007pA004pU007pG007 | AS | 1781 |
| | pC004pU004pA004pC004pA007pA004pA007pA004pC004pC004 | | |
| | p001C004p001A004 | | |
| 148 | G004p001G004p001U004pU004pU007pU004pG007pU007pA007 | SS | 1782 |
| | pG004pC004pA004pU004pU004pU004pU004pU004pA004pU004 | | |
| | A004p001U007p001A004pA004pA004pA007pA004pU004pG004 | AS | 1783 |
| | pC004pU004pA004pC004pA007pA004pA007pA004pC004pC004 | | |
| | p001C004p001A004 | | |
| 149 | U004p001G004p001G004pG004pU004pU004pU004pU004pG007 | SS | 1784 |
| | pU007pA007pG004pC004pA004pU004pU004pU004pU004pU004 | | |
| | pA004pU004 | | |
| | A004p001U007p001A004pA004pA004pA007pA004pU004pG004 | AS | 1785 |
| | pc004pU004pA004pC004pA007pA004pA007pA004pC004pC004 | | |
| | pC004pA004p001G004p001A004 | | |
| 150 | U004p001G004p001G004pG004pU004pU004pU007pU004pG007 | SS | 1786 |
| | pU007pA007pG004pC004pA004pU004pU004pU004pU004pU004 | | |
| | pA004pU004 | | |
| | A004p001U007p001A004pA004pA004pA007pA004pU007pG007 | AS | 1787 |
| | pC004pU004pA004pC004pA007pA004pA007pA004pC004pC004 | | |
| | pC004pA004p001G004p001A004 | | |
| 151 | U004p001G004p001G004pG004pU004pU004pU007pU004pG007 | SS | 1788 |
| | pU007pA007pG004pC004pA004pU004pU004pU004pU004pU004 | | |
| | pA004pU004 | | |
| | A004p001U007p001A004pA004pA004pA007pA004pU004pG004 | AS | 1789 |
| | pC004pU004pA004pC004pA007pA004pA007pA004pC004pC004 | | |
| | pC004pA004p001G004p001A004 | | |
| 152 | G004pG004pU004pU004pU004pU004pG007pU007pA007pG004p | SS | 1790 |
| | C004pA004pU004pU004pU004pU004pU004pA004pU004 | | |
| | pA004pU007pA004pA004pA004pA007pA004pU004pG004pC004 | AS | 1791 |
| | pU004pA004pC004pA007pA004pA007pA004pC004pC004pC004 | | |
| | pA004 | | |
| 152 | G004pG004pU004pU004pU007pU004pG007pU007pA007pG004p | SS | 1792 |
| | C004pA004pU004pU004pU004pU004pU004pA004pU004 | | |
| | pA004pU007pA004pA004pA004pA007pA004pU007pG007pC004 | AS | 1793 |
| | pU004pA004pC004pA007pA004pA007pA004pC004pC004pC004 | | |
| | pA004 | | |
| 153 | G004pG004pU004pU004pU007pU004pG007pU007pA007pG004p | SS | 1794 |
| | C004pA004pU004pU004pU004pU004pU004pA004pU004 | | |
| | PA004pU007pA004pA004pA004pA007pA004pU004pG004pC004 | AS | 1795 |
| | pU004pA004pC004pA007pA004pA007pA004pC004pC004pC004 | | |
| | pA004 | | |
| 154 | U004pG004pG004pG004pU004pU004pU004pU004pG007pU007p | SS | 1796 |
| | A007pG004pC004pA004pU004pU004pU004pU004pU004pA004p | | |
| | U004 | | |
| | pA004pU007pA004pA004pA004pA007pA004pU004pG004pC004 | AS | 1797 |
| | pU004pA004pC004pA007pA004pA007pA004pC004pC004pC004 | | |
| | pA004pG004pA004 | | |
| 154 | U004pG004pG004pG004pU004pU004pU007pU004pG007pU007p | SS | 1798 |
| | A007pG004pC004pA004pU004pU004pU004pU004pU004pA004p | | |
| | U004 | | |
| | PA004pU007pA004pA004pA004pA007pA004pU007pG007pC004 | AS | 1799 |
| | pU004pA004pC004pA007pA004pA007pA004pC004pC004pC004 | | |
| | pA004pG004pA004 | | |
| 155 | U004pG004pG004pG004pU004pU004pU007pU004pG007pU007p | SS | 1800 |
| | A007pG004pC004pA004pU004pU004pU004pU004pU004pA004p | | |
| | U004 | | |
| | PA004pU007pA004pA004pA004pA007pA004pU004pG004pC004 | AS | 1801 |
| | pU004pA004pC004pA007pA004pA007pA004pC004pC004pC004 | | |
| | pA004pG004pA004p | | |
| 156 | G004p001G004p001U004pU004pU004pU004pG007pU007pA007 | SS | 1802 |
| | pG004pC004pA004pU004pU004pU004pU004pU004pA004pU004 | | |
| | pA004p001U007p001A004pA004pA004pA007pA004pU004pG00 | AS | 1803 |
| | 4pC004pU004pA004pC004pA007pA004pA007pA004pC004pC00 | | |
| | 4p001C004p001A004 | | |
| 157 | G004p001G004p001U004pU004pU007pU004pG007pU007pA007 | SS | 1804 |
| | pG004pC004pA004pU004pU004pU004pU004pU004pA004pU004 | | |
| | pA004p001U007p001A004pA004pA004pA007pA004pU007pG00 | AS | 1805 |
| | 7pC004pU004pA004pC004pA007pA004pA007pA004pC004pC00 | | |
| | 4p001C004p001A004 | | |
| 158 | G004p001G004p001U004pU004pU007pU004pG007pU007pA007 | SS | 1806 |
| | pG004pC004pA004pU004pU004pU004pU004pU004pA004pU004 | | |
| | pA004p001U007p001A004pA004pA004pA007pA004pU004pG00 | AS | 1807 |
| | 4pC004pU004pA004pC004pA007pA004pA007pA004pC004pC00 | | |
| | 4p001C004p001A004 | | |
| 159 | U004p001G004p001G004pG004pU004pU004pU004pU004pG007 | SS | 1808 |
| | pU007pA007pG004pC004pA004pU004pU004pU004pU004pU004 | | |
| | pA004pU004 | | |
| | PA004p001U007p001A004pA004pA004pA007pA004pU004pG00 | AS | 1809 |
| | 4pC004pU004pA004pC004pA007pA004pA007pA004pC004pC00 | | |
| | 4pC004pA004p001G004p001A004 | | |
| 160 | U004p001G004p001G004pG004pU004pU004pU007pU004pG007 | SS | 1810 |
| | pU007pA007pG004pC004pA004pU004pU004pU004pU004pU004 | | |
| | pA004pU004 | | |
| | PA004p001U007p001A004pA004pA004pA007pA004pU007pG00 | AS | 1811 |
| | 7pC004pU004pA004pC004pA007pA004pA007pA004pC004pC00 | | |
| | 4pC004pA004p001G004p001A004 | | |
| 161 | U004p001G004p001G004pG004pU004pU004pU007pU004pG007 | SS | 1812 |
| | pU007pA007pG004pC004pA004pU004pU004pU004pU004pU004 | | |
| | pA004pU004 | | |
| | pA004p001U007p001A004pA004pA004pA007pA004pU004pG00 | AS | 1813 |
| | 4pC004pU004pA004pC004pA007pA004pA007pA004pC004pC00 | | |
| | 4pC004pA004p001G004p001A004 | | |
| 162 | G000pU000pG000pA000pC000pU000pU000pU000pU000pU000p | SS | 1814 |
| | A000pA000pA000pA000pU000pA000pA000pA000pA000 | | |
| | U000pU000pU000pU000pA000pU000pU000pU000pU000pA000p | | |
| | A000pA000pA000pA000pG000pU000pC000pA000pC000pC000p | AS | 1815 |
| | A000 | | |
| 163 | U000pG000pG000pU000pG000pA000pC000pU000pU000pU000p | SS | 1816 |
| | U000pU000pA000pA000pA000pA000pU000pA000pA000pA000p | | |
| | A000 | | |
| | U000pU000pU000pU000pA000pU000pU000pU000pU000pA000p | AS | 1817 |
| | A000pA000pA000pA000pG000pU000pC000pA000pC000pC000p | | |
| | A000pU000pA000 | | |
| 164 | G004pU004pG004pA004pC004pU004pU007pU007pU007pU004p | SS | 1818 |
| | A004pA004pA004pA004pU004pA004pA004pA004pA004 | | |
| | U004pU007pU004pU004pA004pU007pU004pU004pU004pA004p | AS | 1819 |
| | A004pA004pA004pA007pG004pU007pC004pA004pC004pC004p | | |
| | A004 | | |
| 165 | G004pU004pG004pA004pC007pU004pU007pU007pU007pU004p | SS | 1820 |
| | A004pA004pA004pA004pU004pA004pA004pA004pA004 | | |
| | U004pU007pU004pU004pA004pU007pU004pU007pU007pA004p | AS | 1821 |
| | A004pA004pA004pA007pG004pU007pC004pA004pC004pC004p | | |
| | A004 | | |
| 166 | G004pU004pG004pA004pC007pU004pU007pU007pU007pU004p | SS | 1822 |
| | A004pA004pA004pA004pU004pA004pA004pA004pA004 | | |
| | U004pU007pU004pU004pA004pU007pU004pU004pU004pA004p | AS | 1823 |
| | A004pA004pA004pA007pG004pU007pC004pA004pC004pC004p | | |
| | A004 | | |
| 167 | U004pG004pG004pU004pG004pA004pC004pU004pU007pU007p | SS | 1824 |
| | U007pU004pA004pA004pA004pA004pU004pA004pA004pA004p | | |
| | A004 | | |
| | U004pU007pU004pU004pA004pU007pU004pU004pU004pA004p | AS | 1825 |
| | A004pA004pA004pA007pG004pU007pC004pA004pC004pC004p | | |
| | A004pU004pA004 | | |
| 168 | U004pG004pG004pU004pG004pA004pC007pU004pU007pU007p | SS | 1826 |
| | U007pU004pA004pA004pA004pA004pU004pA004pA004pA004p | | |
| | A004 | | |
| | U004pU007pU004pU004pA004pU007pU004pU007pU007pA004p | AS | 1827 |
| | A004pA004pA004pA007pG004pU007pC004pA004pC004pC004p | | |
| | A004pU004pA004 | | |
| 169 | U004pG004pG004pU004pG004pA004pC007pU004pU007pU007p | SS | 1828 |
| | U007pU004pA004pA004pA004pA004pU004pA004pA004pA004p | | |
| | A004 | | |
| | U004pU007pU004pU004pA004pU007pU004pU004pU004pA004p | AS | 1829 |
| | A004pA004pA004pA007pG004pU007pC004pA004pC004pC004p | | |
| | A004pU004pA004 | | |
| 170 | G004p001U004p001G004pA004pC004pU004pU007pU007pU007 | SS | 1830 |
| | pU004pA004pA004pA004pA004pU004pA004pA004pA004pA004 | | |
| | U004p001U007p001U004pU004pA004pU007pU004pU004pU004 | AS | 1831 |
| | pA004pA004pA004pA004pA007pG004pU007pC004pA004pC004 | | |
| | p001C004p001A004 | | |
| 171 | G004p001U004p001G004pA004pC007pU004pU007pU007pU007 | SS | 1832 |
| | pU004pA004pA004pA004pA004pU004pA004pA004pA004pA004 | | |
| | U004p001U007p001U004pU004pA004pU007pU004pU007pU007 | AS | 1833 |
| | pA004pA004pA004pA004pA007pG004pU007pC004pA004pC004 | | |
| | p001C004p001A004 | | |
| 172 | G004p001U004p001G004pA004pC007pU004pU007pU007pU007 | SS | 1834 |
| | pU004pA004pA004pA004pA004pU004pA004pA004pA004pA004 | | |
| | U004p001U007p001U004pU004pA004pU007pU004pU004pU004 | AS | 1835 |
| | pA004pA004pA004pA004pA007pG004pU007pC004pA004pC004 | | |
| | p001C004p001A004 | | |
| 173 | U004p001G004p001G004pU004pG004pA004pC004pU004pU007 | SS | 1836 |
| | pU007pU007pU004pA004pA004pA004pA004pU004pA004pA004 | | |
| | pA004pA004 | | |
| | U004p001U007p001U004pU004pA004pU007pU004pU004pU004 | AS | 1837 |
| | pA004pA004pA004pA004pA007pG004pU007pC004pA004pC004 | | |
| | pC004pA004p001U004p001A004 | | |
| 174 | U004p001G004p001G004pU004pG004pA004pC007pU004pU007 | SS | 1838 |
| | pU007pU007pU004pA004pA004pA004pA004pU004pA004pA004 | | |
| | pA004pA004 | | |
| | U004p001U007p001U004pU004pA004pU007pU004pU007pU007 | AS | 1839 |
| | pA004pA004pA004pA004pA007pG004pU007pC004pA004pC004 | | |
| | pC004pA004p001U004p001A004 | | |
| 175 | U004p001G004p001G004pU004pG004pA004pC007pU004pU007 | SS | 1840 |
| | pu007pU007pU004pA004pA004pA004pA004pU004pA004pA004 | | |
| | pA004pA004 | | |
| | U004p001U007p001U004pU004pA004pU007pU004pU004pU004 | AS | 1841 |
| | pA004pA004pA004pA004pA007pG004pU007pC004pA004pC004 | | |
| | pC004pA004p001U004p001A004 | | |
| 176 | G004pU004pG004pA004pC004pU004pU007pU007pU007pU004p | SS | 1842 |
| | A004pA004pA004pA004pU004pA004pA004pA004pA004 | | |
| | PU004pU007pU004pU004pA004pU007pU004pU004pU004pA004 | AS | 1843 |
| | pA004pA004pA004pA007pG004pU007pC004pA004pC004pC004 | | |
| | pA004 | | |
| 177 | G004pU004pG004pA004pC007pU004pU007pU007pU007pU004p | SS | 1844 |
| | A004pA004pA004pA004pU004pA004pA004pA004pA004 | | |
| | PU004pU007pU004pU004pA004pU007pU004pU007pU007pA004 | AS | 1845 |
| | pA004pA004pA004pA007pG004pU007pC004pA004pC004pC004 | | |
| | pA004 | | |
| 178 | G004pU004pG004pA004pC007pU004pU007pU007pU007pU004p | SS | 1846 |
| | A004pA004pA004pA004pU004pA004pA004pA004pA004 | | |
| | PU004pU007pU004pU004pA004pU007pU004pU004pU004pA004 | AS | 1847 |
| | pA004pA004pA004pA007pG004pU007pC004pA004pC004pC004 | | |
| | pA004 | | |
| 179 | U004pG004pG004pU004pG004pA004pC004pU004pU007pU007p | SS | 1848 |
| | U007pU004pA004pA004pA004pA004pU004pA004pA004pA004p | | |
| | A004 | | |
| | PU004pU007pU004pU004pA004pU007pU004pU004pU004pA004 | AS | 1849 |
| | pA004pA004pA004pA007pG004pU007pC004pA004pC004pC004 | | |
| | pA004pU004pA004 | | |
| 180 | U004pG004pG004pU004pG004pA004pC007pU004pU007pU007p | SS | 1850 |
| | U007pU004pA004pA004pA004pA004pU004pA004pA004pA004p | | |
| | A004 | | |
| | PU004pU007pU004pU004pA004pU007pU004pU007pU007pA004 | AS | 1851 |
| | pA004pA004pA004pA007pG004pU007pC004pA004pC004pC004 | | |
| | pA004pU004pA004 | | |
| 181 | U004pG004pG004pU004pG004pA004pC007pU004pU007pU007p | SS | 1852 |
| | U007pU004pA004pA004pA004pA004pU004pA004pA004pA004p | | |
| | A004 | | |
| | PU004pU007pU004pU004pA004pU007pU004pU004pU004pA004 | AS | 1853 |
| | pA004pA004pA004pA007pG004pU007pC004pA004pC004pC004 | | |
| | pA004pU004pA004 | | |
| 182 | G004p001U004p001G004pA004pC004pU004pU007pU007pU007 | SS | 1854 |
| | pU004pA004pA004pA004pA004pU004pA004pA004pA004pA004 | | |
| | PU004p001U007p001U004pU004pA004pU007pU004pU004pU00 | AS | 1855 |
| | 4pA004pA004pA004pA004pA007pG004pU007pC004pA004pC00 | | |
| | 4p001C004p001A004 | | |
| 183 | G004p001U004p001G004pA004pC007pU004pU007pU007pU007 | SS | 1856 |
| | pU004pA004pA004pA004pA004pU004pA004pA004pA004pA004 | | |
| | PU004p001U007p001U004pU004pA004pU007pU004pU007pU00 | AS | 1857 |
| | 7pA004pA004pA004pA004pA007pG004pU007pC004pA004pC00 | | |
| | 4p001C004p001A004 | | |
| 184 | G004p001U004p001G004pA004pC007pU004pU007pU007pU007 | SS | 1858 |
| | pU004pA004pA004pA004pA004pU004pA004pA004pA004pA004 | | |
| | PU004p001U007p001U004pU004pA004pU007pU004pU004pU00 | AS | 1859 |
| | 4pA004pA004pA004pA004pA007pG004pU007pC004pA004pC00 | | |
| | 4p001C004p001A004 | | |
| 185 | U004p001G004p001G004pU004pG004pA004pC004pU004pU007 | SS | 1860 |
| | pU007pU007pU004pA004pA004pA004pA004pU004pA004pA004 | | |
| | pA004pA004 | | |
| | PU004p001U007p001U004pU004pA004pU007pU004pU004pU00 | AS | 1861 |
| | 4pA004pA004pA004pA004pA007pG004pU007pC004pA004pC00 | | |
| | 4pC004pA004p001U004p001A004 | | |
| 186 | U004p001G004p001G004pU004pG004pA004pC007pU004pU007 | SS | 1862 |
| | pU007pU007pU004pA004pA004pA004pA004pU004pA004pA004 | | |
| | pA004pA004 | | |
| | PU004p001U007p001U004pU004pA004pU007pU004pU007pU00 | AS | 1863 |
| | 7pA004pA004pA004pA004pA007pG004pU007pC004pA004pC00 | | |
| | 4pC004pA004p001U004p001A004 | | |

Specific codes in the nucleotide sequences are indicated in above, e.g., Tables A and A-1.

Additional suitable second dsRNAi agent targeting PCSK9 in Table 6b, or variants thereof and synthesis thereof are also described in WO2020/233655, entire contents of which are incorporated herein by reference.

In some embodiments, the second dsRNAi agent may have a structure of siRNA

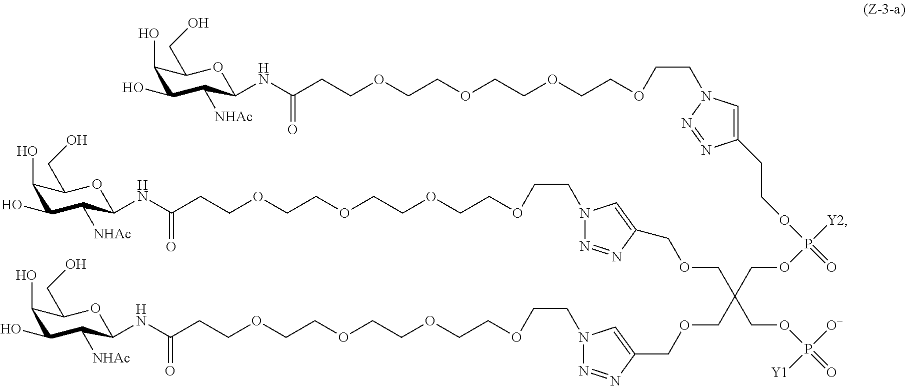

or a pharmaceutically acceptable salt thereof,
wherein at least one of Y1 and Y2 is a dsRNA including any one of PCSK9 siRNA in Table 6c, the dsRNA includes:
   (i) a sense strand; and
   (ii) an antisense strand forming a duplex with the sense strand.

TABLE 6c

| PCSK9 | | | SEQ ID |
|---|---|---|---|
| siRNA | Sequence (5′-3′) | Strand | NO. |
| 187 | A004p001G004p001A004pC004pC004pU004pG007p0004pU007 | SS | 1864 |
| | pU004p0007pG004pC004p0004pU004pU004pU004pG004pU004 | | |
| | pA004pA004p | | |
| | U004p0010007p001A004pC004pA004pA004pA004pA004pG004 | AS | 1865 |
| | pC004pA004pA007pA004pA007pC004pA007pG004pG004pU004 | | |
| | pC004pU004p001A004p001G004p | | |
| 188 | U004p001U004p001U004pU004pG004pC004pU007pU004pU007 | SS | 1866 |
| | pU004pG007pU004pA004pA004pC004pU004pU004pG004pA004 | | |
| | pA004pA004p | | |
| | U004p001U007p001U004pC004pA004pA004pG004pU004pU004 | AS | 1867 |
| | pA004pC004pA007pA004pA007pA004pG007pC004pA004pA004 | | |
| | pA004pA004p001C004p001A004p | | |
| 189 | C004p001U004p001U004pU004pU004pG004pU007pA004pA007 | SS | 1868 |
| | pC004pU007pU004pG004pA004pA004pG004pA004pU004pA004 | | |
| | pU004 pU004 | | |
| | A004p001A007p001U004pA004pU004pC004pU004pU004pC004 | AS | 1869 |
| | pA004pA004pG007pU004pU007pA004pC007pA004pA004pA004 | | |
| | pA004pG004p001C004p001A004 | | |
| 190 | U004p001U004p001U004pU004pG004pU004pA007pG004pC007 | SS | 1870 |
| | pA004pU007pU004pU004pU004pU004pA004pU004pU004pA004 | | |
| | pA004pU004 | | |
| | A004p001U007p001U004pA004pA004pU004pA004pA004pA004 | AS | 1871 |
| | pA004pA004pU007pG004pC007pU004pA007pC004pA004pA004 | | |
| | pA004pA004p001C004p001C004 | | |
| 191 | U004p001U004p001G004pU004pA004pG004pC007pA004pU007 | SS | 1872 |
| | pU004pU007pU004pU004pA004pU004pU004pA004pA004pU004 | | |
| | pA004pU004 | | |
| | A004p001U007p001A004pU004pU004pA004pA004pU004pA004 | AS | 1873 |
| | pA004pA004pA007pA004pU007pG004pC007pU004pA004pC004 | | |
| | pA004pA004p001A004p001A004 | | |
| 192 | C004p001U004p001A004pG004pA004pC004pC007pU004pG007 | SS | 1874 |
| | pU004PT002pU004pU004pG004pC004pU004pU004pU004pU004 | | |
| | pG004pU004 | | |
| | A004p001C007p001A004pA007pA007pA007pG004pC007pA004 | AS | 1875 |
| | pA007pA004pA007pC004pA007pG004pG007pU004pC007pU004 | | |
| | pA004pG004p001A004p001A004 | | |
| 193 | C004p001C004p001U004pC004pA004pC004pC007pA004pA007 | SS | 1876 |
| | pG007pA007pU004pC004pC004pU004pG004pC004pA004pU004 | | |
| | pG004pU004 | | |
| | A004p001C007p001A004pU004pG004pC004pA007pG004pG004 | AS | 1877 |
| | pA004pU004pC007pU004pU007pG004pG007pU004pG004pA004 | | |
| | pG004pG004p001U004p001A004 | | |
| 194 | C004p001A004p001C004pC004pA004pA004pG007pA004pU007 | SS | 1878 |
| | pC007pC007pU004pG004pC004pA004pU004pG004pU004pC004 | | |
| | pU004pU004 | | |
| | A004p001A007p001G004pA004pC004pA004pU007pG004pC004 | AS | 1879 |
| | pA004pG004pG007pA004pU007pC004pU007pU004pG004pG004 | | |
| | pU004pG004p001A004p001G004 | | |
| 195 | C004p001A004p001A004pG004pA004pU004pC007pC004pU007 | SS | 1880 |
| | pG007pC007pA004pU004pG004pU004pC004pU004pU004pC004 | | |
| | pC004pA004 | | |
| | U004p001G007p001G004pA004pA004pG004pA007pC004pA004 | AS | 1881 |
| | pU004pG004pC007pA004pG007pG004pA007pU004pC004pU004 | | |
| | pU004pG004p001G004p001U004 | | |
| 196 | U004p001C004p001C004pU004pG004pG004pC007pU004pU007 | SS | 1882 |
| | pC007pC007pU004pG004pG004pU004pG004pA004pA004pG004 | | |
| | pA004pU004 | | |
| | A004p001U007p001C004pU004pU004pC004pA007pC004pC004 | AS | 1883 |
| | pA004pG004pG007pA004pA007pG004pC007pC004pA004pG004 | | |
| | pG004pA004p001A004p001G004 | | |
| 197 | C004p001U004p001G004pG004pC004pU004pU007pC004pC007 | SS | 1884 |
| | pU007pG007pG004pU004pG004pA004pA004pG004pA004pU004 | | |
| | pG004pA004 | | |
| | U004p001C007p001A004pU004pC004pU004pU007pC004pA004 | AS | 1885 |
| | pC004pC004pA007pG004pG007pA004pA007pG004pC004pC004 | | |
| | pA004pG004p001G004p001A004 | | |
| 198 | G004p001G004p001C004pU004pU004pC004pC007pU004pG007 | SS | 1886 |
| | pG007pU007pG004pA004pA004pG004pA004pU004pG004pA004 | | |
| | pG004pU004 | | |
| | A004p001C007p001U004pC004pA004pU004pC007pU004pU004 | AS | 1887 |
| | pC004pA004pC007pC004pA007pG004pG007pA004pA004pG004 | | |
| | pC004pC004p001A004p001G004 | | |
| 199 | G004p001C004p001U004pU004pC004pC004pU007pG004pG007 | SS | 1888 |
| | pU007pG007pA004pA004pG004pA004pU004pG004pA004pG004 | | |
| | pU004pG004 | | |
| | C004p001A007p001C004pU004pC004pA004pU007pC004pU004 | AS | 1889 |
| | pU004pC004pA007pC004pC007pA004pG007pG004pA004pA004 | | |
| | pG004pC004p001C004p001A004 | | |
| 200 | U004p001G004p001G004pA004pG004pC004pU007pG004pG007 | SS | 1890 |
| | pC007pC007pU004pU004pG004pA004pA004pG004pU004pU004 | | |
| | pG004pC004 | | |
| | G004p001C007p001A004pA004pC004pU004pU007pC004pA004 | AS | 1891 |
| | pA004pG004pG007pC004pC007pA004pG007pC004pU004pC004 | | |
| | pC004pA004p001G004p001C004 | | |
| 201 | A004p001G004p001U004pU004pG004pC004pC007pC004pC007 | SS | 1892 |
| | pA007pU007pG004pU004pC004pG004pA004pC004pU004pA004 | | |
| | pC004pA004 | | |
| | U004p001G007p001U004pA004pG004pU004pC007pG004pA004 | AS | 1893 |
| | pC004pA004pU007pG004pG007pG004pG007pC004pA004pA004 | | |
| | pC004pU004p001U004p001C004 | | |
| 202 | U004p001G004p001C004pC004pC004pC004pA007pU004pG007 | SS | 1894 |
| | pU007pC007pG004pA004pC004pU004pA004pC004pA004pU004 | | |
| | pC004pG004 | | |
| | C004p001G007p001A004pU004pG004pU004pA007pG004pU004 | AS | 1895 |
| | pC004pG004pA007pC004pA007pU004pG007pG004pG004pG004 | | |
| | pC004pA004p001A004p001C004 | | |
| 203 | C004p001G004p001G004pU004pA004pC004pC007pG004pG007 | SS | 1896 |
| | pG007pC007pG004pG004pA004pU004pG004pA004pA004pU004 | | |
| | pA004pC004 | | |
| | G004p001U007p001A004pU004pU004pC004pA007pU004pC004 | AS | 1897 |
| | pC004pG004pC007pC004pC007pG004pG007pU004pA004pC004 | | |
| | pC004pG004p001U004p001G004p | | |
| 204 | G004p001G004p001U004pA004pC004pC004pG007pG004pG007 | SS | 1898 |
| | pC007pG007pG004pA004pU004pG004pA004pA004pU004pA004 | | |
| | pC004pC004 | | |
| | G004p001G007p001U004pA004pU004pU004pC007pA004pU004 | AS | 1899 |
| | pC004pC004pG007pC004pC007pC004pG007pG004pU004pA004 | | |
| | pC004pC004p001G004p001U004 | | |
| 205 | G004p001G004p001C004pA004pG004pC004pC007pU004pG007 | SS | 1900 |
| | pG007pU007pG004pG004pA004pG004pG004pU004pG004pU004 | | |
| | pA004pU004 | | |
| | A004p001U007p001A004pC004pA004pC004pC007pU004pC004 | AS | 1901 |
| | pC004pA004pC007pC004pA007pG004pG007pC004pU004pG004 | | |
| | pC004pC004p001U004p001C004 | | |
| 206 | C004p001A004p001G004pC004pC004pU004pG007pG004pU007 | SS | 1902 |
| | pG007pG007pA004pG004pG004pU004pG004pU004pA004pU004 | | |
| | pC004pU004 | | |
| | A004p001G007p001A004pU004pA004pC004pA007pC004pC004 | AS | 1903 |
| | pU004pC004pC007pA004pC007pC004pA007pG004pG004pC004 | | |
| | pU004pG004p001C004p001C004 | | |
| 207 | A004p001G004p001C004pC004pU004pG004pG007pU004pG007 | SS | 1904 |
| | pG007pA007pG004pG004pU004pG004pU004pA004pU004pC004 | | |
| | pU004pC004 | | |
| | G004p001A007p001G004pA004pU004pA004pC007pA004pC004 | AS | 1905 |
| | pC004pU004pC007pC004pA007pC004pC007pA004pG004pG004 | | |
| | pC004pU004p001G004p001C004 | | |
| 208 | G004p001C004p001C004pU004pG004pG004pU007pG004pG007 | SS | 1906 |
| | pA007pG007pG004pU004pG004pU004pA004pU004pC004pU004 | | |
| | pC004pC004 | | |
| | G004p001G007p001A004pG004pA004pU004pA007pC004pA004 | AS | 1907 |
| | pC004pC004pU007pC004pC007pA004pC007pC004pA004pG004 | | |
| | pG004pC004p001U004p001G004 | | |
| 209 | G004p001U004p001A004pU004pC004pU004pC007pC004pU007 | SS | 1908 |
| | pA007pG007pA004pC004pA004pC004pC004pA004pG004pC004 | | |
| | pA004pU004 | | |
| | A004p001U007p001G004pC004pU004pG004pG007pU004pG004 | AS | 1909 |
| | pU004pC004pU007pA004pG007pG004pA007pG004pA004pU004 | | |
| | pA004pC004p001A004p001C004 | | |
| 210 | A004p001U004p001C004pU004pC004pC004pU007pA004pG007 | SS | 1910 |
| | pA007pC007pA004pC004pC004pA004pG004pC004pA004pU004 | | |
| | pA004pC004 | | |
| | G004p001U007p001A004pU004pG004pC004pU007pG004pG004 | AS | 1911 |
| | pU004pG004pU007pC004pU007pA004pG007pG004pA004pG004 | | |
| | pA004pU004p001A004p001C004 | | |
| 211 | U004p001C004p001C004pU004pA004pG004pA007pC004pA007 | SS | 1912 |
| | pc007pC007pA004pG004pC004pA004pU004pA004pC004pA004 | | |
| | pG004pA004 | | |
| | U004p001C007p001U004pG004pU004pA004pU007pG004pC004 | AS | 1913 |
| | pU004pG004pG007pU004pG007pU004pC007pU004pA004pG004 | | |
| | pG004pA004p001G004p001A004 | | |
| 212 | C004p001U004p001A004pG004pA004pC004pA007pC004pC007 | SS | 1914 |
| | pA007pG007pC004pA004pU004pA004pC004pA004pG004pA004 | | |
| | pG004pU004 | | |
| | A004p001C007p001U004pC004pU004pG004pU007pA004pU004 | AS | 1915 |
| | pG004pC004pU007pG004pG007pU004pG007pU004pC004pU004 | | |
| | pA004pG004p001G004p001A004 | | |
| 213 | A004p001U004p001G004pG004pU004pC004pA007pC004pC007 | SS | 1916 |
| | pG007pA007pC004pU004pU004pC004pG004pA004pG004pA004 | | |
| | pA004pU004 | | |
| | A004p001U007p001U004pC004pU004pC004pG007pA004pA004 | AS | 1917 |
| | pG004pU004pC007pG004pG007pU004pG007pA004pC004pC004 | | |
| | pA004pU004p001G004p001A004 | | |
| 214 | U004p001C004p001A004pC004pC004pG004pA007pC004pU007 | SS | 1918 |
| | pU007pC007pG004pA004pG004pA004pA004pU004pG004pU004 | | |
| | pG004pC004 | | |
| | G004p001C007p001A004pC004pA004pU004pU007pC004pU004 | AS | 1919 |
| | pC004pG004pA007pA004pG007pU004pC007pG004pG004pU004 | | |
| | pG004pA004p001C004p001C004 | | |
| 215 | C004p001C004p001U004pC004pA004pU004pA007pG004pG007 | SS | 1920 |
| | pC007pC007pU004pG004pG004pA004pG004pU004pU004pU004 | | |
| | pA004pU004 | | |
| | A004p001U007p001A004pA004pA004pC004pU007pC004pC004 | AS | 1921 |
| | pA004pG004pG007pC004pC007pU004pA007pU004pG004pA004 | | |
| | pG004pG004p001G004p001U004 | | |
| 216 | G004p001G004p001C004pC004pU004pG004pG007pA004pG007 | SS | 1922 |
| | pU007pU007pU004pA004pU004pU004pC004pG004pG004pA004 | | |
| | pA004pA004 | | |
| | U004p001U007p001U004pC004pC004pG004pA007pA004pU004 | AS | 1923 |
| | pA004pA004pA007pC004pU007pC004pC007pA004pG004pG004 | | |
| | pC004pC004p001U004p001A004 | | |
| 217 | G004p001C004p001C004pU004pG004pG004pA007pG004pU007 | SS | 1924 |
| | pU007pU007pA004pU004pU004pC004pG004pG004pA004pA004 | | |
| | pA004pA004 | | |
| | U004p001U007p001U004pU004pC004pC004pG007pA004pA004 | AS | 1925 |
| | pU004pA004pA007pA004pC007pU004pC007pC004pA004pG004 | | |
| | pG004pC004p001C004p001U004 | | |
| 218 | U004p001U004p001G004pU004pG004pU004pC007pA004pC007 | SS | 1926 |
| | pA007pG007pA004pG004pU004pG004pG004pG004pA004pC004 | | |
| | pA004pU004 | | |
| | A004p001U007p001G004pU004pC004pC004pC007pA004pC004 | AS | 1927 |
| | pU004pC004pU007pG004pU007pG004pA007pC004pA004pC004 | | |
| | pA004pA004p001A004p001G004 | | |
| 219 | C004p001U004p001G004pA004pU004pC004pC007pA004pC007 | SS | 1928 |
| | pU007pU007pC004pU004pC004pU004pG004pC004pC004pA004 | | |
| | pA004pA004 | | |
| | U004p001U007p001U004pG004pG004pC004pA007pG004pA004 | AS | 1929 |
| | pG004pA004pA007pG004pU007pG004pG007pA004pU004pC004 | | |
| | pA004pG004p001U004p001C004 | | |
| 220 | A004p001U004p001C004pC004pA004pC004pU007pU004pC007 | SS | 1930 |
| | pU007pC007pU004pG004pC004pC004pA004pA004pA004pG004 | | |
| | pA004pU004 | | |
| | A004p001U007p001C004pU004pU004pU004pG007pG004pC004 | AS | 1931 |
| | pA004pG004pA007pG004pA007pA004pG007pU004pG004pG004 | | |
| | pA004pU004p001C004p001A004 | | |
| 221 | U004p001C004p001U004pC004pU004pG004pC007pC004pA007 | SS | 1932 |
| | pA007pA007pG004pA004pU004pG004pU004pC004pA004pU004 | | |
| | pC004pA004 | | |
| | U004p001G007p001A004pU004pG004pA004pC007pA004pU004 | AS | 1933 |
| | pC004pU004pU007pU004pG007pG004pC007pA004pG004pA004 | | |
| | pG004pA004p001A004p001G004 | | |
| 222 | U004p001G004p001U004pC004pA004pU004pC007pA004pA007 | SS | 1934 |
| | pU007pG007pA004pG004pG004pC004pC004pU004pG004pG004 | | |
| | pU004pU004 | | |
| | A004p001A007p001C004pC004pA004pG004pG007pC004pC004 | AS | 1935 |
| | pU004pC004pA007pU004pU007pG004pA007pU004pG004pA004 | | |
| | pC004pA004p001U004p001C004 | | |
| 223 | A004p001G004p001C004pU004pG004pU004pU007pU004pU007 | SS | 1936 |
| | pG007pC007pA004pG004pG004pA004pC004pU004pG004pU004 | | |
| | pA004pU004 | | |
| | A004p001U007p001A004pC004pA004pG004pU007pC004pC004 | AS | 1937 |
| | pU004pG004pC007pA004pA007pA004pA007pC004pA004pG004 | | |
| | pC004pU004p001G004p001C004 | | |
| 224 | C004p001U004p001G004pU004pU004pU004pU007pG004pC007 | SS | 1938 |
| | pA007pG007pG004pA004pC004pU004pG004pU004pA004pU004 | | |
| | pG004pG004 | | |
| | C004p001C007p001A004pU004pA004pC004pA007pG004pU004 | AS | 1939 |
| | pC004pC004pU007pG004pC007pA004pA007pA004pA004pC004 | | |
| | pA004pG004p001C004p001U004 | | |
| 225 | G004p001G004p001A004pC004pU004pG004pU007pA004pU007 | SS | 1940 |
| | pG007pG007pU004pC004pA004pG004pC004pA004pC004pA004 | | |
| | pC004pU004 | | |
| | A004p001G007p001U004pG004pU004pG004pC007pU004pG004 | AS | 1941 |
| | pA004pC004pC007pA004pU007pA004pC007pA004pG004pU004 | | |
| | pC004pC004p001U004p001G004 | | |
| 226 | A004p001C004p001U004pG004pU004pA004pU007pG004pG007 | SS | 1942 |
| | pU007pC007pA004pG004pC004pA004pC004pA004pC004pU004 | | |
| | pC004pG004 | | |
| | C004p001G007p001A004pG004pU004pG004pU007pG004pC004 | AS | 1943 |
| | pU004pG004pA007pC004pC007pA004pU007pA004pC004pA004 | | |
| | pG004pU004p001C004p001C004 | | |
| 227 | C004p001C004p001C004pA004pG004pG004pU007pC004pU007 | SS | 1944 |
| | pG007pG007pA004pA004pU004pG004pC004pA004pA004pA004 | | |
| | pG004pU004 | | |
| | A004p001C007p001U004pU004pU004pG004pC007pA004pU004 | AS | 1945 |
| | pU004pC004pC007pA004pG007pA004pC007pC004pU004pG004 | | |
| | pG004pG004p001G004p001C004 | | |
| 228 | U004p001C004p001U004pG004pG004pA004pA007pU004pG007 | SS | 1946 |
| | pC007pA007pA004pA004pG004pU004pC004pA004pA004pG004 | | |
| | pG004pA004 | | |
| | U004p001C007p001C004pU004pU004pG004pA007pC004pU004 | AS | 1947 |
| | pU004pU004pG007pC004pA007pU004pU007pC004pC004pA004 | | |
| | pG004pA004p001C004p001C004 | | |
| 229 | U004p001A004p001G004pA004pC004pA004pA007pC004pA007 | SS | 1948 |
| | pC007pG007pU004pG004pU004pG004pU004pA004pG004pU004 | | |
| | pC004pA004 | | |
| | U004p001G007p001A004pC004pU004pA004pC007pA004pC004 | AS | 1949 |
| | pA004pC004pG007pU004pG007pU004pU007pG004pU004pC004 | | |
| | pU004pA004p001C004p001G004 | | |
| 230 | C004p001U004p001G004pG004pG004pG004pC007pU004pG007 | SS | 1950 |
| | pA007pG007pC004pU004pU004pU004pA004pA004pA004pA004 | | |
| | pU004pG004 | | |
| | C004p001A007p001U004pU004pU004pU004pA007pA004pA004 | AS | 1951 |
| | pG004pC004pU007pC004pA007pG004pC007pC004pC004pC004 | | |
| | pA004pG004p001C004p001C004 | | |
| 231 | U004p001G004p001G004pG004pG004pC004pU007pG004pA007 | SS | 1952 |
| | pG007pC007pU004pU004pU004pA004pA004pA004pA004pU004 | | |
| | pG004pG004 | | |
| | C004p001C007p001A004pU004pU004pU004pU007pA004pA004 | AS | 1953 |
| | pA004pG004pC007pU004pC007pA004pG007pC004pC004pC004 | | |
| | pC004pA004p001G004p001C004 | | |
| 232 | C004p001C004p001C004pU004pC004pA004pC007pU004pG007 | SS | 1954 |
| | pU007pG007pG004pG004pG004pC004pA004pU004pU004pU004 | | |
| | pC004pA004 | | |
| | U004p001G007p001A004pA004pA004pU004pG007pC004pC004 | AS | 1955 |
| | pC004pC004pA007pC004pA007pG004pU007pG004pA004pG004 | | |
| | pG004pG004p001A004p001G004 | | |
| 233 | C004p001A004p001C004pU004pG004pU004pG007pG004pG007 | SS | 1956 |
| | pG007pC007pA004pU004pU004pU004pC004pA004pC004pC004 | | |
| | pA004pU004 | | |
| | A004p001U007p001G004pG004pU004pG004pA007pA004pA004 | AS | 1957 |
| | pU004pG004pC007pC004pC007pC004pA007pC004pA004pG004 | | |
| | pU004pG004p001A004p001G004 | | |
| 234 | C004p001U004p001G004pU004pG004pG004pG007pG004pC007 | SS | 1958 |
| | pA007pU007pU004pU004pC004pA004pC004pC004pA004pU004 | | |
| | pU004pC004 | | |
| | G004p001A007p001A004pU004pG004pG004pU007pG004pA004 | AS | 1959 |
| | pA004pA004pU007pG004pC007pC004pC007pC004pA004pC004 | | |
| | pA004pG004p001U004p001G004 | | |
| 235 | U004p001G004p001G004pG004pG004pC004pA007pU004pU007 | SS | 1960 |
| | pU007pC007pA004pC004pC004pA004pU004pU004pC004pA004 | | |
| | pA004pA004 | | |
| | U004p001U007p001U004pG004pA004pA004pU007pG004pG004 | AS | 1961 |
| | pU004pG004pA007pA004pA007pU004pG007pC004pC004pC004 | | |
| | pC004pA004p001C004p001A004 | | |
| 236 | G004p001C004p001A004pU004pU004pU004pC007pA004pC007 | SS | 1962 |
| | pc007pA007pU004pU004pC004pA004pA004pA004pC004pA004 | | |
| | pG004pG004 | | |
| | C004p001C007p001U004pG004pU004pU004pU007pG004pA004 | AS | 1963 |
| | pA004pU004pG007pG004pU007pG004pA007pA004pA004pU004 | | |
| | pG004pC004p001C004p001C004 | | |
| 237 | U004p001U004p001U004pA004pU004pU004pG007pA004pG007 | SS | 1964 |
| | pc007pU007pC004pU004pU004pG004pU004pU004pC004pC004 | | |
| | pG004pU004 | | |
| | A004p001C007p001G004pG004pA004pA004pC007pA004pA004 | AS | 1965 |
| | pG004pA004pG007pC004pU007pC004pA007pA004pU004pA004 | | |
| | pA004pA004p001A004p001G004 | | |
| 238 | C004p001C004p001C004pU004pC004pA004pU007pC004pU007 | SS | 1966 |
| | pc007pC007pA004pG004pC004pU004pA004pA004pC004pU004 | | |
| | pG004pU004 | | |
| | A004p001C007p001A004pG004pU004pU004pA007pG004pC004 | AS | 1967 |
| | pU004pG004pG007pA004pG007pA004pU007pG004pA004pG004 | | |
| | pG004pG004p001C004p001C004 | | |
| 239 | A004p001C004p001U004pG004pA004pG004pC007pC004pA007 | SS | 1968 |
| | pG007pA007pA004pA004pC004pG004pC004pA004pG004pA004 | | |
| | pU004pU004 | | |
| | A004p001A007p001U004pC004pU004pG004pC007pG004pU004 | AS | 1969 |
| | pU004pU004pC007pU004pG007pG004pC007pU004pC004pA004 | | |
| | pG004pU004p001U004p001C004 | | |
| 240 | U004p001G004p001A004pG004pC004pC004pA007pG004pA007 | SS | 1970 |
| | pA007pA007pC004pG004pC004pA004pG004pA004pU004pU004 | | |
| | pG004pG004 | | |
| | C004p001C007p001A004pA004pU004pC004pU007pG004pC004 | AS | 1971 |
| | pG004pU004pU007pU004pC007pU004pG007pG004pC004pU004 | | |
| | pC004pA004p001G004p001U004 | | |
| 241 | G004p001A004p001A004pG004pC004pC004pA007pA004pG007 | SS | 1972 |
| | pC007pC007pU004pC004pU004pU004pC004pU004pU004pA004 | | |
| | pC004pU004 | | |
| | A004p001G007p001U004pA004pA004pG004pA007pA004pG004 | AS | 1973 |
| | pA004pG004pG007pC004pU007pU004pG007pG004pC004pU004 | | |
| | pU004pC004p001A004p001G004 | | |
| 242 | A004p001G004p001C004pC004pA004pA004pG007pC004pC007 | SS | 1974 |
| | pU007pC007pU004pU004pC004pU004pU004pA004pC004pU004 | | |
| | pU004pC004 | | |
| | G004p001A007p001A004pG004pU004pA004pA007pG004pA004 | AS | 1975 |
| | pA004pG004pA007pG004pG007pC004pU007pU004pG004pG004 | | |
| | pc004pU004p001U004p001C004 | | |
| 243 | C004p001C004p001C004pA004pA004pG004pC007pA004pA007 | SS | 1976 |
| | pG007pC007pA004pG004pA004pC004pA004pU004pU004pU004 | | |
| | pA004pU004 | | |
| | A004p001U007p001A004pA004pA004pU004pG007pU004pC004 | AS | 1977 |
| | pU004pG004pC007pU004pU007pG004pC007pU004pU004pG004 | | |
| | pG004pG004p001U004p001G004 | | |
| 244 | C004p001C004p001A004pA004pG004pC004pA007pA004pG007 | SS | 1978 |
| | pC007pA007pG004pA004pC004pA004pU004pU004pU004pA004 | | |
| | pU004pC004 | | |
| | G004p001A007p001U004pA004pA004pA004pU007pG004pU004 | AS | 1979 |
| | pC004pU004pG007pC004pU007pU004pG007pC004pU004pU004 | | |
| | pG004pG004p001G004p001U004 | | |
| 245 | U004p001U004p001U004pU004pG004pG004pG007pU004pC007 | SS | 1980 |
| | pU007pG007pU004pC004pC004pU004pC004pU004pC004pU004 | | |
| | pG004pU004 | | |
| | A004p001C007p001A004pG004pA004pG004pA007pG004pG004 | AS | 1981 |
| | pA004pC004pA007pG004pA007pC004pC007pC004pA004pA004 | | |
| | pA004pA004p001G004p001A004 | | |
| 246 | U004p001C004p001U004pG004pU004pC004pC007pU004pC007 | SS | 1982 |
| | pU007pC007pU004pG004pU004pU004pG004pC004pC004pU004 | | |
| | pU004pU004 | | |
| | A004p001A007p001A004pG004pG004pC004pA007pA004pC004 | AS | 1983 |
| | pA004pG004pA007pG004pA007pG004pG007pA004pC004pA004 | | |
| | pG004pA004p001C004p001C004 | | |
| 247 | C004p001U004p001G004pU004pC004pC004pU007pC004pU007 | SS | 1984 |
| | pC007pU007pG004pU004pU004pG004pC004pC004pU004pU004 | | |
| | pU004pU004 | | |
| | A004p001A007p001A004pA004pG004pG004pC007pA004pA004 | AS | 1985 |
| | pC004pA004pG007pA004pG007pA004pG007pG004pA004pC004 | | |
| | pA004pG004p001A004p001C004 | | |
| 248 | U004p001G004p001U004pC004pC004pU004pC007pU004pC007 | SS | 1986 |
| | pU007pG007pU004pU004pG004pC004pC004pU004pU004pU004 | | |
| | pU004pU004 | | |
| | A004p001A007p001A004pA004pA004pG007pC004pA004pA004 | AS | 1987 |
| | pC004pA007pG004pA007pG004pA007pG004pG004pA004pC004 | | |
| | pA004p001G004p001A004 | | |
| 249 | G004p001U004p001C004pC004pU004pC004pU007pC004pU007 | SS | 1988 |
| | pG007pU007pU004pG004pC004pC004pU004pU004pU004pU004 | | |
| | pU004pA004 | | |
| | U004p001A007p001A004pA004pA004pA004pG007pG004pC004 | AS | 1989 |
| | pA004pA004pC007pA004pG007pA004pG007pA004pG004pG004 | | |
| | pA004pC004p001A004p001G004 | | |
| 250 | C004p001C004p001U004pC004pU004pC004pU007pG004pU007 | SS | 1990 |
| | pU007pG007pC004pC004pU004pU004pU004pU004pU004pA004 | | |
| | pC004pA004 | | |
| | U004p001G007p001U004pA004pA004pA004pA007pA004pG004 | AS | 1991 |
| | pG004pC004pA007pA004pC007pA004pG007pA004pG004pA004 | | |
| | pG004pG004p001A004p001C004 | | |
| 251 | G004p001U004p001U004pG004pC004pC004pU007pU004pU007 | SS | 1992 |
| | pU007pU007pA004pC004pA004pG004pC004pC004pA004pA004 | | |
| | pC004pU004 | | |
| | A004p001G007p001U004pU004pG004pG004pC007pU004pG004 | AS | 1993 |
| | pU004pA004pA007pA004pA007pA004pG007pG004pC004pA004 | | |
| | pA004pC004p001A004p001G004 | | |
| 252 | U004p001G004p001C004pC004pU004pU004pU007pU004pU007 | SS | 1994 |
| | pA007pC007pA004pG004pC004pC004pA004pA004pC004pU004 | | |
| | pU004pU004 | | |
| | A004p001A007p001A004pG004pU004pU004pG007pG004pC004 | AS | 1995 |
| | pU004pG004pU007pA004pA007pA004pA007pA004pG004pG004 | | |
| | pC004pA004p001A004p001C004 | | |
| 253 | C004p001C004p001U004pU004pU004pU004pU007pA004pC007 | SS | 1996 |
| | pA007pG007pC004pC004pA004pA004pC004pU004pU004pU004 | | |
| | pU004pC004 | | |
| | G004p001A007p001A004pA004pA004pG004pU007pU004pG004 | AS | 1997 |
| | pG004pC004pU007pG004pU007pA004pA007pA004pA004pA004 | | |
| | pG004pG004p001C004p001A004 | | |
| 254 | U004p001U004p001U004pU004pA004pC004pA007pG004pC007 | SS | 1998 |
| | pC007pA007pA004pC004pU004pU004pU004pU004pC004pU004 | | |
| | pA004pG004 | | |
| | C004p001U007p001A004pG004pA004pA004pA007pA004pG004 | AS | 1999 |
| | pU004pU004pG007pG004pC007pU004pG007pU004pA004pA004 | | |
| | pA004pA004p001A004p001G004 | | |
| 255 | G004p001C004p001C004pA004pA004pC004pU007pU004pU007 | SS | 2000 |
| | pU007pC007pU004pA004pG004pA004pC004pC004pU004pG004 | | |
| | pU004pU004 | | |
| | A004p001A007p001C004pA004pG004pG004pU007pC004pU004 | AS | 2001 |
| | pA004pG004pA007pA004pA007pA004pG007pU004pU004pG004 | | |
| | pG004pC004p001U004p001G004 | | |
| 256 | C004p001C004p001A004pA004pC004pU004pU007pU004pU007 | SS | 2002 |
| | pC007pU007pA004pG004pA004pC004pC004pU004pG004pU004 | | |
| | pU004pU004 | | |
| | A004p001A007p001A004pC004pA004pG004pG007pU004pC004 | AS | 2003 |
| | pU004pA004pG007pA004pA007pA004pA007pG004pU004pU004 | | |
| | pG004pG004p001C004p001U004 | | |
| 257 | C004p001A004p001A004pC004pU004pU004pU007pU004pC007 | SS | 2004 |
| | pU007pA007pG004pA004pC004pC004pU004pG004pU004pU004 | | |
| | pU004pU004 | | |
| | A004p001A007p001A004pA004pC004pA004pG007pG004pU004 | AS | 2005 |
| | pC004pU004pA007pG004pA007pA004pA007pA004pG004pU004 | | |
| | pU004pG004p001G004p001C004 | | |
| 258 | A004p001C004p001U004pU004pU004pU004pC007pU004pA007 | SS | 2006 |
| | pG007pA007pC004pC004pU004pG004pU004pU004pU004pU004 | | |
| | pG004pC004 | | |
| | G004p001C007p001A004pA004pA004pA004pC007pA004pG004 | AS | 2007 |
| | pG004pU004pC007pU004pA007pG004pA007pA004pA004pA004 | | |
| | pG004pU004p001U004p001G004 | | |
| 259 | U004p001U004p001U004pU004pC004pU004pA007pG004pA007 | SS | 2008 |
| | pC007pC007pU004pG004pU004pU004pU004pU004pG004pC004 | | |
| | pU004pU004 | | |
| | A004p001A007p001G004pC004pA004pA004pA007pA004pC004 | AS | 2009 |
| | pA004pG004pG007pU004pC007pU004pA007pG004pA004pA004 | | |
| | pA004pA004p001G004p001U004 | | |
| 260 | U004p001U004p001U004pC004pU004pA004pG007pA004pC007 | SS | 2010 |
| | pC007pU007pG004pU004pU004pU004pU004pG004pC004pU004 | | |
| | pU004pU004 | | |
| | A004p001A007p001A004pG004pC004pA004pA007pA004pA004 | AS | 2011 |
| | pC004pA004pG007pG004pU007pC004pU007pA004pG004pA004 | | |
| | pA004pA004p001A004p001G004 | | |
| 261 | U004p001C004p001U004pA004pG004pA004pC007pC004pU007 | SS | 2012 |
| | pG007pU007pU004pU004pU004pG004pC004pU004pU004pU004 | | |
| | pU004pG004 | | |
| | C004p001A007p001A004pA004pA004pG004pC007pA004pA004 | AS | 2013 |
| | pA004pA004pC007pA004pG007pG004pU007pC004pU004pA004 | | |
| | pG004pA004p001A004p001A004 | | |
| 262 | C004p001U004p001A004pG004pA004pC004pC007pU004pG007 | SS | 2014 |
| | pU007pU007pU004pU004pG004pC004pU004pU004pU004pU004 | | |
| | pG004pU004 | | |
| | A004p001C007p001A004pA004pA004pA004pG007pC004pA004 | AS | 2015 |
| | pA004pA004pA007pC004pA007pG004pG007pU004pC004pU004 | | |
| | pA004pG004p001A004p001A004 | | |
| 263 | U004p001A004p001G004pA004pC004pC004pU007pG004pU007 | SS | 2016 |
| | pU007pU007pU004pG004pC004pU004pU004pU004pU004pG004 | | |
| | pU004pA004 | | |
| | U004p001A007p001C004pA004pA004pA004pA007pG004pC004 | AS | 2017 |
| | pA004pA004pA007pA004pC007pA004pG007pG004pU004pC004 | | |
| | pU004pA004p001G004p001A004 | | |
| 264 | A004p001G004p001A004pC004pC004pU004pG007pU004pU007 | SS | 2018 |
| | pU007pU007pG004pC004pU004pU004pU004pU004pG004pU004 | | |
| | pA004pA004 | | |
| | U004p001U007p001A004pC004pA004pA004pA007pA004pG004 | AS | 2019 |
| | pC004pA004pA007pA004pA007pC004pA007pG004pG004pU004 | | |
| | pC004pU004p001A004p001G004 | | |
| 265 | G004p001A004p001C004pC004pU004pG004pU007pU004pU007 | SS | 2020 |
| | pU007pG007pC004pU004pU004pU004pU004pG004pU004pA004 | | |
| | pA004pC004 | | |
| | G004p001U007p001U004pA004pC004pA004pA007pA004pA004 | AS | 2021 |
| | pG004pC004pA007pA004pA007pA004pC007pA004pG004pG004 | | |
| | pU004pC004p001U004p001A004 | | |
| 266 | A004p001C004p001C004pU004pG004pU004pU007pU004pU007 | SS | 2022 |
| | pG007pC007pU004pU004pU004pU004pG004pU004pA004pA004 | | |
| | pC004pU004 | | |
| | A004p001G007p001U004pU004pA004pC004pA007pA004pA004 | AS | 2023 |
| | pA004pG004pC007pA004pA007pA004pA007pC004pA004pG004 | | |
| | pG004pU004p001C004p001U004 | | |
| 267 | C004p001U004p001G004pU004pU004pU004pU007pG004pC007 | SS | 2024 |
| | pU007pU007pU004pU004pG004pU004pA004pA004pC004pU004 | | |
| | pU004pG004 | | |
| | C004p001A007p001A004pG004pU004pU004pA007pC004pA004 | AS | 2025 |
| | pA004pA004pA007pG004pC007pA004pA007pA004pA004pC004 | | |
| | pA004pG004p001G004p001U004 | | |
| 268 | G004p001U004p001U004pU004pU004pG004pC007pU004pU007 | SS | 2026 |
| | pU007pU007pG004pU004pA004pA004pC004pU004pU004pG004 | | |
| | pA004pA004 | | |
| | U004p001U007p001C004pA004pA004pG004pU007pU004pA004 | AS | 2027 |
| | pC004pA004pA007pA004pA007pG004pC007pA004pA004pA004 | | |
| | pA004pC004p001A004p001G004 | | |
| 269 | U004p001U004p001U004pG004pC004pU004pU007pU004pU007 | SS | 2028 |
| | pG007pU007pA004pA004pC004pU004pU004pG004pA004pA004 | | |
| | pG004pA004 | | |
| | U004p001C007p001U004pU004pC004pA004pA007pG004pU004 | AS | 2029 |
| | pU004pA004pC007pA004pA007pA004pA007pG004pC004pA004 | | |
| | pA004pA004p001A004p001C004 | | |
| 270 | U004p001U004p001G004pC004pU004pU004pU007pU004pG007 | SS | 2030 |
| | pU007pA007pA004pC004pU004pU004pG004pA004pA004pG004 | | |
| | pA004pU004 | | |
| | A004p001U007p001C004pU004pU004pC004pA007pA004pG004 | AS | 2031 |
| | pU004pU004pA007pC004pA007pA004pA007pA004pG004pC004 | | |
| | pA004pA004p001A004p001A004 | | |
| 271 | C004p001U004p001U004pU004pU004pG004pU007pA004pA007 | SS | 2032 |
| | pC007pU007pU004pG004pA004pA004pG004pA004pU004pA004 | | |
| | pU004pU004 | | |
| | A004p001A007p001U004pA004pU004pC004pU007pU004pC004 | AS | 2033 |
| | pA004pA004pG007pU004pU007pA004pC007pA004pA004pA004 | | |
| | pA004pG004p001C004p001A004 | | |
| 272 | U004p001U004p001U004pU004pG004pU004pA007pA004pC007 | SS | 2034 |
| | pU007pU007pG004pA004pA004pG004pA004pU004pA004pU004 | | |
| | pU004pU004 | | |
| | A004p001A007p001A004pU004pA004pU004pC007pU004pU004 | AS | 2035 |
| | pC004pA004pA007pG004pU007pU004pA007pC004pA004pA004 | | |
| | pA004pA004p001G004p001C004 | | |
| 273 | U004p001U004p001U004pG004pU004pA004pA007pC004pU007 | SS | 2036 |
| | pU007pG007pA004pA004pG004pA004pU004pA004pU004pU004 | | |
| | pU004pA004 | | |
| | U004p001A007p001A004pA004pU004pA004pU007pC004pU004 | AS | 2037 |
| | pU004pC004pA007pA004pG007pU004pU007pA004pC004pA004 | | |
| | pA004pA004p001A004p001G004 | | |
| 274 | U004p001A004p001A004pC004pU004pU004pG007pA004pA007 | SS | 2038 |
| | pG007pA007pU004pA004pU004pU004pU004pA004pU004pU004 | | |
| | pC004pU004 | | |
| | A004p001G007p001A004pA004pU004pA004pA007pA004pU004 | AS | 2039 |
| | pA004pU004pC007pU004pU007pC004pA007pA004pG004pU004 | | |
| | pU004pA004p001C004p001A004 | | |
| 275 | A004p001A004p001C004pU004pU004pG004pA007pA004pG007 | SS | 2040 |
| | pA007pU007pA004pU004pU004pU004pA004pU004pU004pC004 | | |
| | pU004pG004 | | |
| | C004p001A007p001G004pA004pA004pU004pA007pA004pA004 | AS | 2041 |
| | pU004pA004pU007pC004pU007pU004pC007pA004pA004pG004 | | |
| | pU004pU004p001A004p001C004 | | |
| 276 | A004p001A004p001G004pA004pU004pA004pU007pU004pU007 | SS | 2042 |
| | pA007pU007pU004pC004pU004pG004pG004pG004pU004pU004 | | |
| | pU004pU004 | | |
| | A004p001A007p001A004pA004pC004pC004pC007pA004pG004 | AS | 2043 |
| | pA004pA004pU007pA004pA007pA004pU007pA004pU004pC004 | | |
| | pU004pU004p001C004p001A004 | | |
| 277 | A004p001G004p001A004pU004pA004pU004pU007pU004pA007 | SS | 2044 |
| | pU007pU007pC004pU004pG004pG004pG004pU004pU004pU004 | | |
| | pU004pG004 | | |
| | C004p001A007p001A004pA004pA004pC004pC007pC004pA004 | AS | 2045 |
| | pG004pA004pA007pU004pA007pA004pA007pU004pA004pU004 | | |
| | pC004pU004p001U004p001C004 | | |
| 278 | A004p001U004p001A004pU004pU004pU004pA007pU004pU007 | SS | 2046 |
| | pc007pU007pG004pG004pG004pU004pU004pU004pU004pG004 | | |
| | pU004pA004 | | |
| | U004p001A007p001C004pA004pA004pA004pA007pC004pC004 | AS | 2047 |
| | pC004pA004pG007pA004pA007pU004pA007pA004pA004pU004 | | |
| | pA004pU004p001C004p001U004 | | |
| 279 | U004p001A004p001U004pU004pU004pA004pU007pU004pC007 | SS | 2048 |
| | pU007pG007pG004pG004pU004pU004pU004pU004pG004pU004 | | |
| | pA004pG004 | | |
| | C004p001U007p001A004pC004pA004pA004pA007pA004pC004 | AS | 2049 |
| | pC004pC004pA007pG004pA007pA004pU007pA004pA004pA004 | | |
| | pU004pA004p001U004p001C004 | | |
| 280 | U004p001U004p001C004pU004pG004pG004pG007pU004pU007 | SS | 2050 |
| | pU007pU007pG004pU004pA004pG004pC004pA004pU004pU004 | | |
| | pU004pU004 | | |
| | A004p001A007p001A004pA004pU004pG004pC007pU004pA004 | AS | 2051 |
| | pC004pA004pA007pA004pA007pC004pC007pC004pA004pG004 | | |
| | pA004pA004p001U004p001A004 | | |
| 281 | G004p001G004p001U004pU004pU004pU004pG007pU004pA007 | SS | 2052 |
| | pG007pC007pA004pU004pU004pU004pU004pU004pA004pU004 | | |
| | pU004pA004 | | |
| | U004p001A007p001A004pU004pA004pA004pA007pA004pA004 | AS | 2053 |
| | pU004pG004pC007pU004pA007pC004pA007pA004pA004pA004 | | |
| | pC004pC004p001C004p001A004 | | |
| 282 | G004p001U004p001U004pU004pU004pG004pU007pA004pG007 | SS | 2054 |
| | pC007pA007pU004pU004pU004pU004pU004pA004pU004pU004 | | |
| | pA004pA004 | | |
| | U004p001U007p001A004pA004pU004pA004pA007pA004pA004 | AS | 2055 |
| | pA004pU004pG007pC004pU007pA004pC007pA004pA004pA004 | | |
| | pA004pC004p001C004p001C004 | | |
| 283 | U004p001U004p001U004pU004pG004pU004pA007pG004pC007 | SS | 2056 |
| | pA007pU007pU004pU004pU004pU004pA004pU004pU004pA004 | | |
| | pA004pU004 | | |
| | A004p001U007p001U004pA004pA004pU004pA007pA004pA004 | AS | 2057 |
| | pA004pA004pU007pG004pC007pU004pA007pC004pA004pA004 | | |
| | pA004pA004p001C004p001C004 | | |
| 284 | A004p001U004p001U004pU004pU004pU004pA007pU004pU007 | SS | 2058 |
| | pA007pA007pU004pA004pU004pG004pG004pU004pG004pA004 | | |
| | pC004pU004 | | |
| | A004p001G007p001U004pC004pA004pC004pC007pA004pU004 | AS | 2059 |
| | pA004pU004pU007pA004pA007pU004pA007pA004pA004pA004 | | |
| | pA004pU004p001G004p001C004 | | |
| 285 | U004p001U004p001U004pU004pU004pA004pU007pU004pA007 | SS | 2060 |
| | pA007pU007pA004pU004pG004pG004pU004pG004pA004pC004 | | |
| | pU004pU004 | | |
| | A004p001A007p001G004pU004pC004pA004pC007pC004pA004 | AS | 2061 |
| | pU004pA004pU007pU004pA007pA004pU007pA004pA004pA004 | | |
| | pA004pA004p001U004p001G004 | | |
| 286 | U004p001U004p001U004pU004pG004pC004pU007pU004pU007 | SS | 2062 |
| | pU007pG007pU004pA004pA004pC004pU004pU004pG004pA004 | | |
| | pA004pG004 | | |
| | C004p001U007p001U004pC004pA004pA004pG007pU004pU004 | AS | 2063 |
| | pA004pC004pA007pA004pA007pA004pG007pC004pA004pA004 | | |
| | pA004pA004p001C004p001A004 | | |
| 287 | U004p001U004p001G004pU004pA004pG004pC007pA004pU007 | SS | 2064 |
| | pU007pU007pU004pU004pA004pU004pU004pA004pA004pU004 | | |
| | pA004pU004 | | |
| | A004p001U007p001A004pU004pU004pA004pA007pU004pA004 | AS | 2065 |
| | pA004pA004pA007pA004pU007pG004pC007pU004pA004pC004 | | |
| | pA004pA004p001A004p001A004 | | |

Specific codes in the nucleotide sequences are indicated in above, e.g., Tables A and A-1.

In some embodiments, in Formula (Z-3-a), Y1 is the dsRNA as described in Table 6c, and Y2 is hydroxy group or a salt. In some embodiments, in Formula (Z-3-a), Y2 is the dsRNA as described in Table 6c, and Y1 is hydroxy group or a salt. In some embodiments, in Formula (Z-3-a), each Y1 and Y2 is independently the dsRNA as described in Table 6c.

Additional suitable second dsRNAi agent targeting PCSK9 in Table 6c and synthesis thereof are also described in WO202/3049294, entire contents of which are incorporated herein by reference.

In some embodiments, the second dsRNAi agent may have a structure of

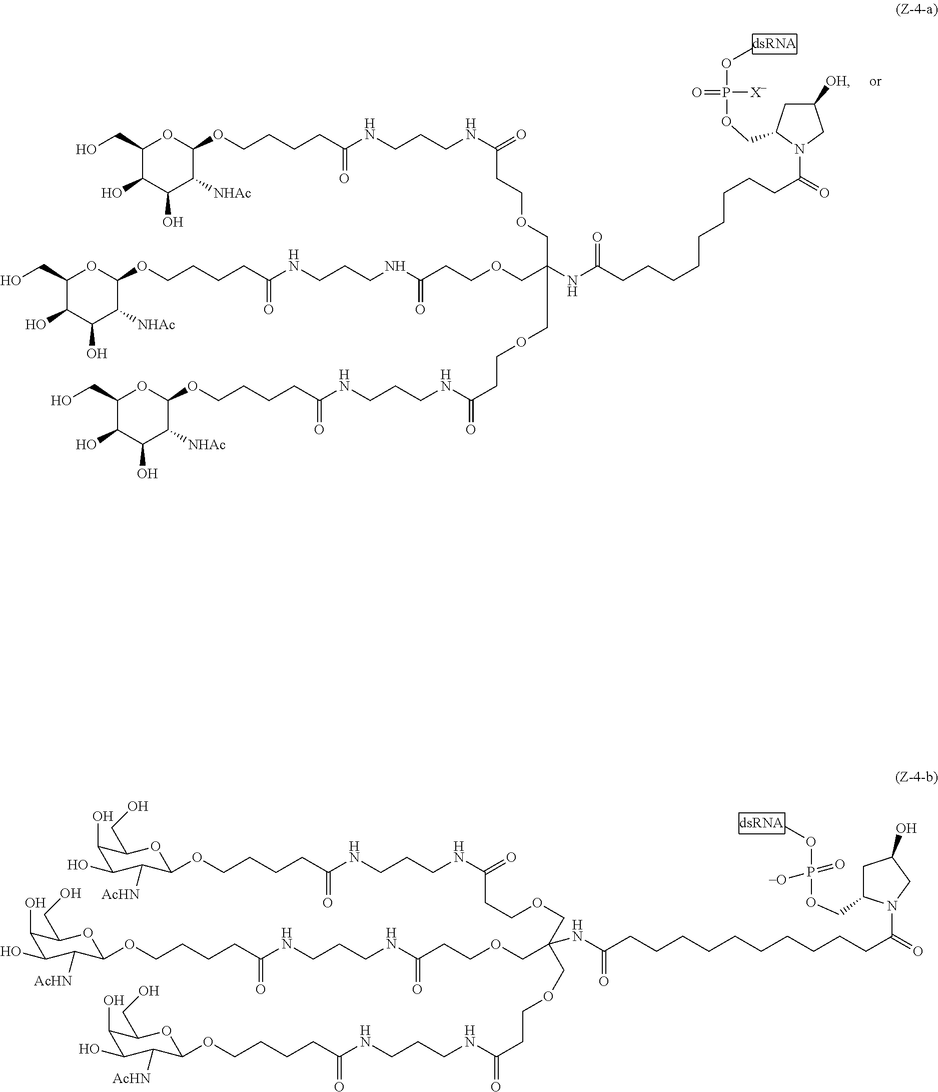

wherein the dsRNA includes any one of PCSK9 siRNA in Table 6d, the dsRNA includes:
   (i) a sense strand; and
   (ii) an antisense strand forming a duplex with the sense strand.

TABLE 6d

| PCSK9 | | | SEQ ID |
|---|---|---|---|
| SiRNA | Sequence (5′-3′) | Strand | NO |
| 288 | U007p001G004p001U007pC004pC007pU004pC007pU004pC007 | AS | 2066 |
| | pU007pG007pU004pU007pG004pC007pC004pU007pU004pU007 | | |
| | pU004pU007 | | |
| | A004p001A007p001A004pA007pA004pG007pG004pC007pA004 | SS | 2067 |
| | pA007pC004pA004pG004pA007pG004pA007pG004pG007pA004 | | |
| | pC007pA004p001G004p001A004 | | |
| 289 | G007p001U004p001C007pC004pU007pC004pU007pC004pU007 | AS | 2068 |
| | pG007pU007pU004pG007pC004pC007pU004pU007pU004pU007 | | |
| | pU004pA007 | | |
| | U004p001A007p001A004pA007pA004pA007pG004pG007pC004 | SS | 2069 |
| | pA007pA004pC004pA004pG007pA004pG007pA004pG007pG004 | | |
| | pA007pC004p001A004p001G004 | | |
| 290 | A007p001G004p001A007pC004pC007pU004pG007pU004pU007 | AS | 2070 |
| | pU007pU007pG004pC007pU004pU007pU004pU007pG004pU007 | | |
| | pA004pA007 | | |
| | U004p001U007p001A004pC007pA004pA007pA004pA007pG004 | SS | 2071 |
| | pC007pA004pA004pA007pC004pA007pG004pG007pU004pC007 | | |
| | pU004p001A004p001G004 | | |
| 291 | G007p001A004p001C007pC004pU007pG004pU007pU004pU007 | AS | 2072 |
| | pU007pG007pC004pU007pU004pU007pU004pG007pU004pA007 | | |
| | pA004pC007 | | |
| | G004p001U007p001U004pA007pC004pA007pA004pA007pA004 | SS | 2073 |
| | pG007pC004pA004pA007pA004pC007pA004pG007pG004pU007 | | |
| | pC004p001U004p001A004 | | |
| 292 | A007p001C004p001C007pU004pG007pU004pU007pU004pU007 | AS | 2074 |
| | pG007pC007pU004pU007pU004pU007pG004pU007pA004pA007 | | |
| | pc004pU007 | | |
| | A004p001G007p001U004pU007pA004pC007pA004pA007pA004 | SS | 2075 |
| | pA007pG004pC004pA004pA007pA004pA007pC004pA007pG004 | | |
| | pG007pU004p001C004p001U004 | | |
| 293 | C007p001U004p001G007pU004pU007pU004pU007pG004pC007 | AS | 2076 |
| | pU007pU007pU004pU007pG004pU007pA004pA007pC004pU007 | | |
| | pU004pG007 | | |
| | C004p001A007p001A004pG007pU004pU007pA004pC007pA004 | SS | 2077 |
| | pA007pA004pA004pG004pC007pA004pA007pA004pA007pC004 | | |
| | pA007pG004p001G004p001U004 | | |
| 294 | U007p001U004p001U007pG004pU007pA004pA007pC004pU007 | AS | 2078 |
| | pU007pG007pA004pA007pG004pA007pU004pA007pU004pU007 | | |
| | pU004pA007 | | |
| | U004p001A007p001A004pA007pU004pA007pU004pC007pU004 | SS | 2079 |
| | pU007pC004pA004pA004pG007pU004pU007pA004pC007pA004 | | |
| | pA007pA004p001A004p001G004 | | |
| 295 | U007p001U004p001G007p0004pA007pA004pC007pU004pU007 | AS | 2080 |
| | pG007pA007pA004pG007pA004pU007pA004p0007p0004pU007 | | |
| | pA004pU007 | | |
| | A004p0010007p001A004pA007pA004pU007pA004pU007pC004 | SS | 2081 |
| | pU007pU004pC004pA004pA007pG004pU007pU004pA007pC004 | | |
| | pA007pA004p001A004p001A004 | | |
| 296 | U007p001G004p0010007pA004pA007pC004pU007p0004pG007 | AS | 2082 |
| | pA007pA007pG004pA007pU004pA007pU004pU007pU004pA007 | | |
| | pU004p0007 | | |
| | A004p001A007p001U004pA007pA004pA007pU004pA007pU004 | SS | 2083 |
| | pC007pU004pU004pC004pA007pA004pG007pU004p0007pA004 | | |
| | pC007pA004p001A004p001A004 | | |
| 297 | G007p001U004p001A007pA004pC007pU004p0007pG004pA007 | AS | 2084 |
| | pA007pG007pA004p0007pA004pU007pU004p0007pA004pU007 | | |
| | p0004pC007 | | |
| | G004p001A007p001A004pU007pA004pA007pA004p0007pA004 | SS | 2085 |
| | p0007pC004pU004p0004pC007pA004pA007pG004pU007pU004 | | |
| | pA007pC004p001A004p001A004 | | |
| 298 | A007p001U004p001A007pU004pU007pU004pA007p0004pU007 | AS | 2086 |
| | pc007pU007pG004pG007pG004pU007pU004pU007p0004pG007 | | |
| | pU004pA007 | | |
| | U004p001A007p001C004pA007pA004pA007pA004pC007pC004 | SS | 2087 |
| | pC007pA004pG004pA004pA007pU004pA007pA004pA007pU004 | | |
| | pA007pU004p001C004p001U004 | | |
| 299 | U007p001U004p0010007pA004pU007pU004pC007p0004pG007 | AS | 2088 |
| | pG007pG007pU004pU007pU004pU007pG004pU007pA004pG007 | | |
| | pC004pA007 | | |
| | U004p001G007p001C004p0007pA004pC007pA004pA007pA004 | SS | 2089 |
| | pA007pC004pC004pC004pA007pG004pA007pA004pU007pA004 | | |
| | pA007pA004p001U004p001A004 | | |
| 300 | A007p001U004p0010007pC004pU007pG004pG007pG004p0007 | AS | 2090 |
| | pU007pU007pU004pG007p0004pA007pG004pC007pA004pU007 | | |
| | pU004pU007 | | |
| | A004p001A007p001A004p0007pG004pC007p0004pA007pC004 | SS | 2091 |
| | pA007pA004pA004pA004pC007pC004pC007pA004pG007pA004 | | |
| | pA007pU004p001A004p001A004 | | |
| 301 | C007p001U004p001G007pG004pG007pU004pU007pU004p0007 | AS | 2092 |
| | pG007pU007pA004pG007pC004pA007pU004p0007pU004p0007 | | |
| | pU004pA007 | | |
| | U004p001A007p001A004pA007pA004pA007pU004pG007pC004 | SS | 2093 |
| | pU007pA004pC004pA004pA007pA004pA007pC004pC007pC004 | | |
| | pA007pG004p001A004p001A004 | | |
| 302 | U007p001G004p001G007pG004pU007pU004p0007p0004pG007 | AS | 2094 |
| | p0007pA007pG004pC007pA004pU007pU004p0007p0004pU007 | | |
| | pA004pU007 | | |
| | A004p0010007p001A004pA007pA004pA007pA004pU007pG004 | SS | 2095 |
| | pC007pU004pA004pC004pA007pA004pA007pA004pC007pC004 | | |
| | pC007pA004p001G004p001A004 | | |
| 303 | G007p001G004p001G007pC004pU007pG004pA007pG004pC007 | AS | 2096 |
| | pU007pU007pU004pA007pA004pA007pA004pU007pG004pG007 | | |
| | pU004pU007 | | |
| | A004p001A007p001C004pC007pA004pU007pU004p0007p0004 | SS | 2097 |
| | pA007pA004pA004pG004pC007pU004pC007pA004pG007pC004 | | |
| | pC007pC004p001C004p001A004 | | |
| 304 | A007p001U004p001A007pC004pC007pU004pG007pU004pU007 | AS | 2098 |
| | pU007pU007pG004pC007pU004pU007pU004p0007pG004pU007 | | |
| | pA004pA007 | | |
| | U004p0010007p001A004pC007pA004pA007pA004pA007pG004 | SS | 2099 |
| | pC007pA004pA004pA007pC004pA007pG004pG007pU004pC007 | | |
| | p004p001A004p001G004 | | |
| 305 | A007p001G004p001A007pC004pC007pU004pG007p0004pU007 | AS | 2100 |
| | pU007pU007pG004pC007pU004pU007pU004p0007pG004pU007 | | |
| | pA004pA007 | | |
| | U004p0010007p001A004pC007pA004pA007pA004pA007pG004 | SS | 2101 |
| | pC007pA004pA004pA007pC004pA007pG004pG007p0004pC007 | | |
| | pU004p001A004p001G004 | | |
| 306 | A007p001G004p001A007pC004pC007pU004pG007p0004pU007 | AS | 2102 |
| | pU007pU007pG004pC007pU004p0007pU004p0007pG004pU007 | | |
| | pA004pA007 | | |
| | U004p0010007p001A004pC007pA004pA007pA004pA007pG004 | SS | 2103 |
| | pC007pA004pA004pA007pC004pA007pG004pG007pU004pC007 | | |
| | pU004p001A004p001G004 | | |
| 307 | A007p001G004p001A007pC004pC007pU004pG007pU004pU007 | AS | 2104 |
| | pU007pU007pG004pC007pU004pU007pU004p0007pG004pU007 | | |
| | pA004pA007 | | |
| | U004p0010007p001A004pC007pA004pA007pA004pA007pG004 | SS | 2105 |
| | pC007pA004pA004pA007pC004pA007pG004pG007p0004pC007 | | |
| | pU004p001A004p001G004 | | |
| 308 | A007p001G004p001A007pC004pC007pU004pG007pU004p0007 | AS | 2106 |
| | pU007pU007pG004pC007p0004pU007pU004p0007pG004pU007 | | |
| | pA004pA007 | | |
| | U004p0010007p001A004pC007pA004pA007pA004pA007pG004 | SS | 2107 |
| | pC007pA004pA004pA007pC004pA007pG004pG007pU004pC007 | | |
| | pU004p001A004p001G004 | | |
| 309 | A007p001G004p001A007pC004pC007pU004pG007p0004pU007 | AS | 2108 |
| | pU007pU007pU007pG004pC007pU004pU007pU004pU007pG004 | | |
| | pU007pA004pA007pG004pC004pA004pG004pC004pC004pG002 | | |
| | pA002pG004pG004pC004pU004p001G004p001C004 | | |
| | U004p0010007p001A004pC007pA004pA007pA004pA007pG004 | SS | 2109 |
| | pC007pA004pA004pA007pC004pA007pG004pG007pU004pC007 | | |
| | pU004p001A004p001G004 | | |
| 310 | A007p001G004p001A007pC004pC007pU004pG007pU004pU007 | AS | 2110 |
| | pU007pU007pG004pC007pU004pU007pU004pU007pG004pU007 | | |
| | pA004pA007 | | |
| | U004p0010007p001A004pC007pA004pA007pA004pA007pG004 | SS | 2111 |
| | pC007pA004pA004pA007pC004pA007pG004pG007pU004pC007 | | |
| | pU004p001A007p001G004 | | |
| 311 | A007p001G004p001A007pC004pC007pU004pG007p0004pU007 | AS | 2112 |
| | pU007pU007pG004pC007pU004pU004pU004pU004pG004pU004 | | |
| | pA004pA004 | | |
| | U004p0010007p001A004pC007pA004pA004pA007pG004pC007 | SS | 2113 |
| | pA004pA004pA007pC004pA007pG004pG004pU004pC007pU004 | | |
| | p001A004p001G004 | | |
| 312 | A004p001G004p001A004pC004pC004pU004pG007p0004pU007 | AS | 2114 |
| | p0004pU004pG004pC004pU004pU004pU004pU004p@004pU004 | | |
| | pA004pA004 | | |
| | U004p0010007p001A004pC007pA004pA004pA007pG004pC007 | SS | 2115 |
| | pA004pA004pA007pC004pA007pG004pG004pU004pC007p0004 | | |
| | p001A004p001G004 | | |
| 313 | A004p001G004p001A004pC004pC004pU004pG007pU004pU007 | AS | 2116 |
| | p0004pU004pG004pC004pU004pU004pU004pU004pG004pU004 | | |
| | pA004pA004 | | |
| | U004p0010007p001A004pC007pA007pA007pA007pA007pG004 | SS | 2117 |
| | pC007pA004pA007pA004pA007pC004pA007pG004pG007pU004 | | |
| | pC004pU004p001A004p001G004 | | |
| 314 | A004p001G004p001A004pC004pC004pU004pG007p0004pU007 | AS | 2118 |
| | pU004pT002pG004pC004pU004pU004pU004p0004pG004pU004 | | |
| | pA004pA004 | | |
| | U004p0010007p001A004pC007pA007pA007pA007pA007pG004 | SS | 2119 |
| | pC007pA004pA007pA004pA007pC004pA007pG004pG007pU004 | | |
| | pC004pU004p001A004p001G004 | | |
| 315 | C007p001U004p001G007p0004pU007pU004p0007pG004pC007 | AS | 2120 |
| | pU007p0007pU004p0007pG004pU007pA004pA007pC004pU007 | | |
| | pU004pG007 | | |
| | C004p001A007p001A004pG007pU004pU007pA004pC007pA004 | SS | 2121 |
| | pA007pA004pA004pG004pC007pA004pA007pA004pA007pC004 | | |
| | pA007pG004p001G007p001U004 | | |
| 316 | C007p001U004p001G007pU004pU007pU004pU007pG004pC007 | AS | 2122 |
| | pU007pU007pU004pU007pG004pU004pA004pA004pC004pU004 | | |
| | pU004pG004 | | |
| | C004p001A007p001A004pG007pU004pU004pA004pC007pA004 | SS | 2123 |
| | pA007pA004pA004pG004pC007pA004pA007pA004pA004pC004 | | |
| | pA007pG004p001G004p001U004 | | |
| 317 | C004p001U004p001G004p0004pU004pU004pU007pG004pC007 | AS | 2124 |
| | pU004pU004pU004pU004pG004pU004pA004pA004pC004p0004 | | |
| | pU004pG004 | | |
| | C004p001A007p001A004pG007pU004pU004pA004pC007pA004 | SS | 2125 |
| | pA007pA004pA004pG004pC007pA004pA007pA004pA004pC004 | | |
| | pA007pG004p001G004p001U004 | | |
| 318 | C004p001U004p001G004pU004pU004pU004pU007pG004pC007 | AS | 2126 |
| | pU004pU004pU004pU004pG004pU004pA004pA004pC004pU004 | | |
| | pU004pG004 | | |
| | C004p001A007p001A004pG007pU007pU007pA004pC007pA004 | SS | 2127 |
| | pA007pA004pA007pG004pC007pA004pA007pA004pA007pC004 | | |
| | pA004pG004p001G004p001U004 | | |
| 319 | C004p001U004p001G004p0004pU004pU004pU007pG004pC007 | AS | 2128 |
| | pU004pT002pU004p0004pG004pU004pA004pA004pC004pU004 | | |
| | pU004pG004 | | |
| | C004p001A007p001A004pG007pU007pU007pA004pC007pA004 | SS | 2129 |
| | pA007pA004pA007pG004pC007pA004pA007pA004pA007pC004 | | |
| | pA004pG004p0016004p001U004 | | |
| 320 | C004p001U004p001G004pU004pU004pU004pU007pG004pC007 | AS | 2130 |
| | p0007pU007pU004p0004pG004p0004pA004pA004pC004pU004 | | |
| | pU004pG004 | | |
| | C004p001A007p001A004pG004pU004pU007pA004pC007pA007 | SS | 2131 |
| | pA004pA004pA004pG004pC007pA004pA007pA004pA004pC004 | | |
| | pA004pG004p001G004p001U004 | | |
| 321 | C004p001U004p001G004p0004pU004pU004p0007pG004pC007 | AS | 2132 |
| | p0007pU007pU004pU004pG004pU004pA004pA004pC004pU004 | | |
| | pU004pG004 | | |
| | C004p001A007p001A004pG004pU004pU007pA004pC004pA004 | SS | 2133 |
| | pA004pA004pG004pC007pA004pA007pA004pA004pC004pA004 | | |
| | pG004p001G004p001U004 | | |
| 322 | C004p001U004p001G004p0004pU004pU004p0007pG004pC007 | AS | 2134 |
| | pU007p0007pU004p0004p@004pU004pA004pA004pC004pU004 | | |
| | pU004pG004 | | |
| | C004p001A007p001A004pG004pU004pU004pA004pC004pA004 | SS | 2135 |
| | pA004pA004pG004pC007pA004pA007pA004pA004pC004pA004 | | |
| | pG004p001G004p001U004 | | |

Specific codes in the nucleotide sequences are indicated in above, e.g., Tables A and A-1.

Additional suitable second dsRNAi agent targeting PCSK9 in Table 6d, or variants thereof and synthesis thereof are also described in WO2023/134609, entire contents of which are incorporated herein by reference.

In some embodiments, the second dsRNAi agent may have a structure of

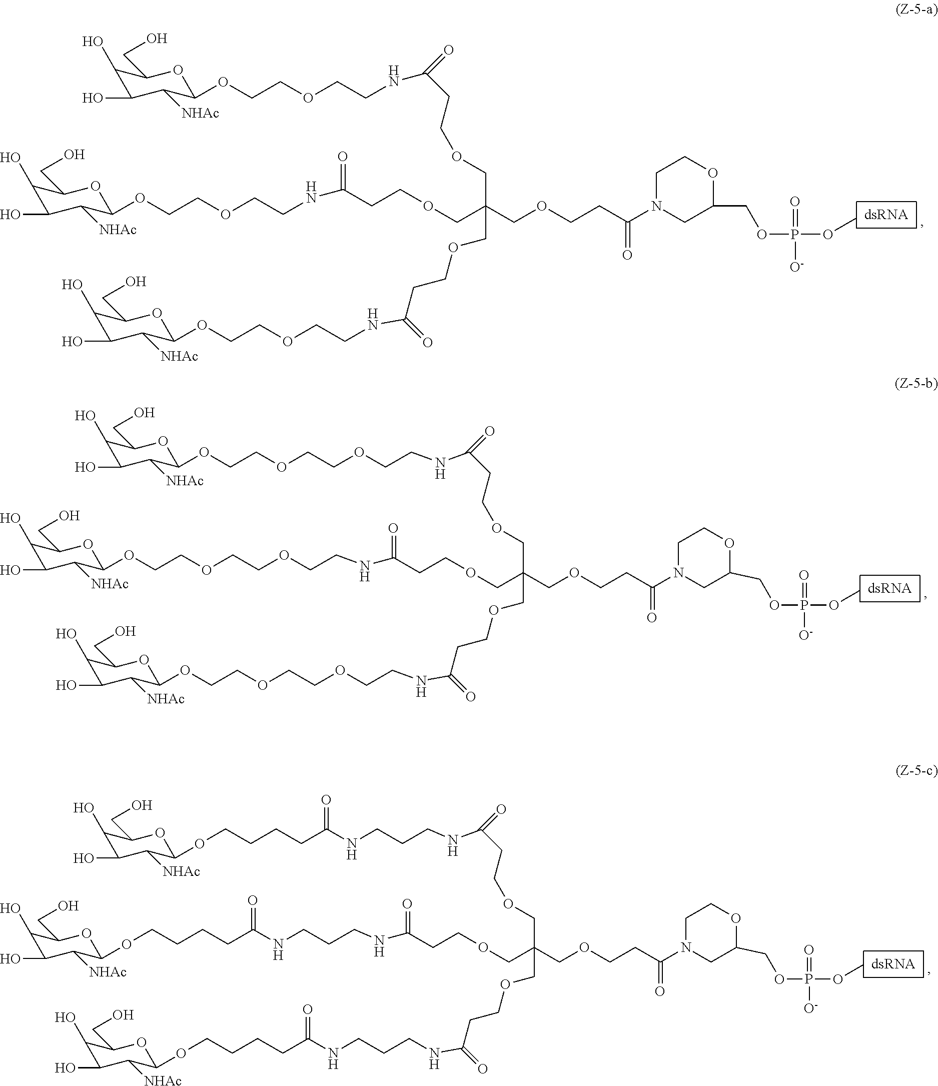

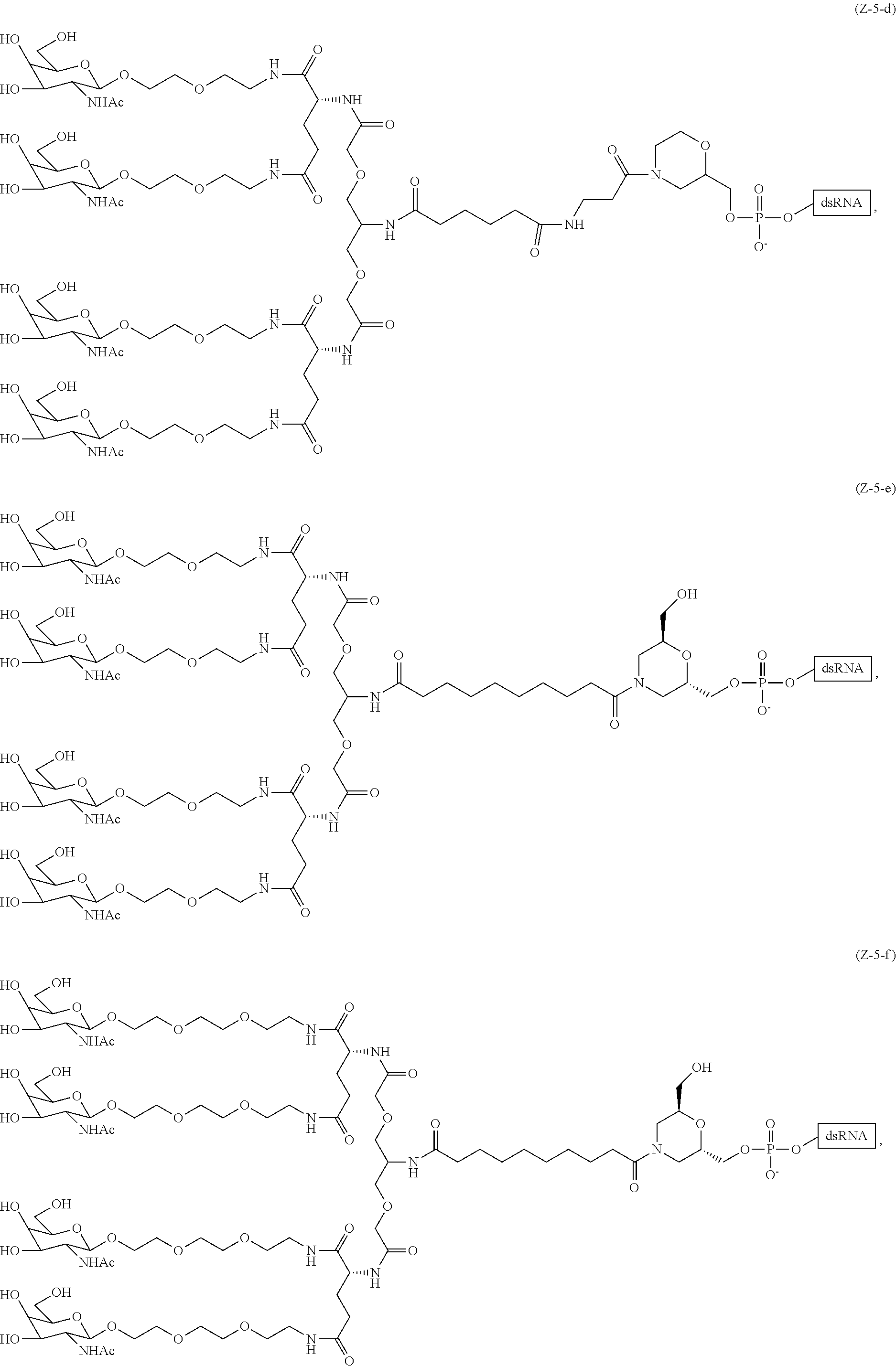

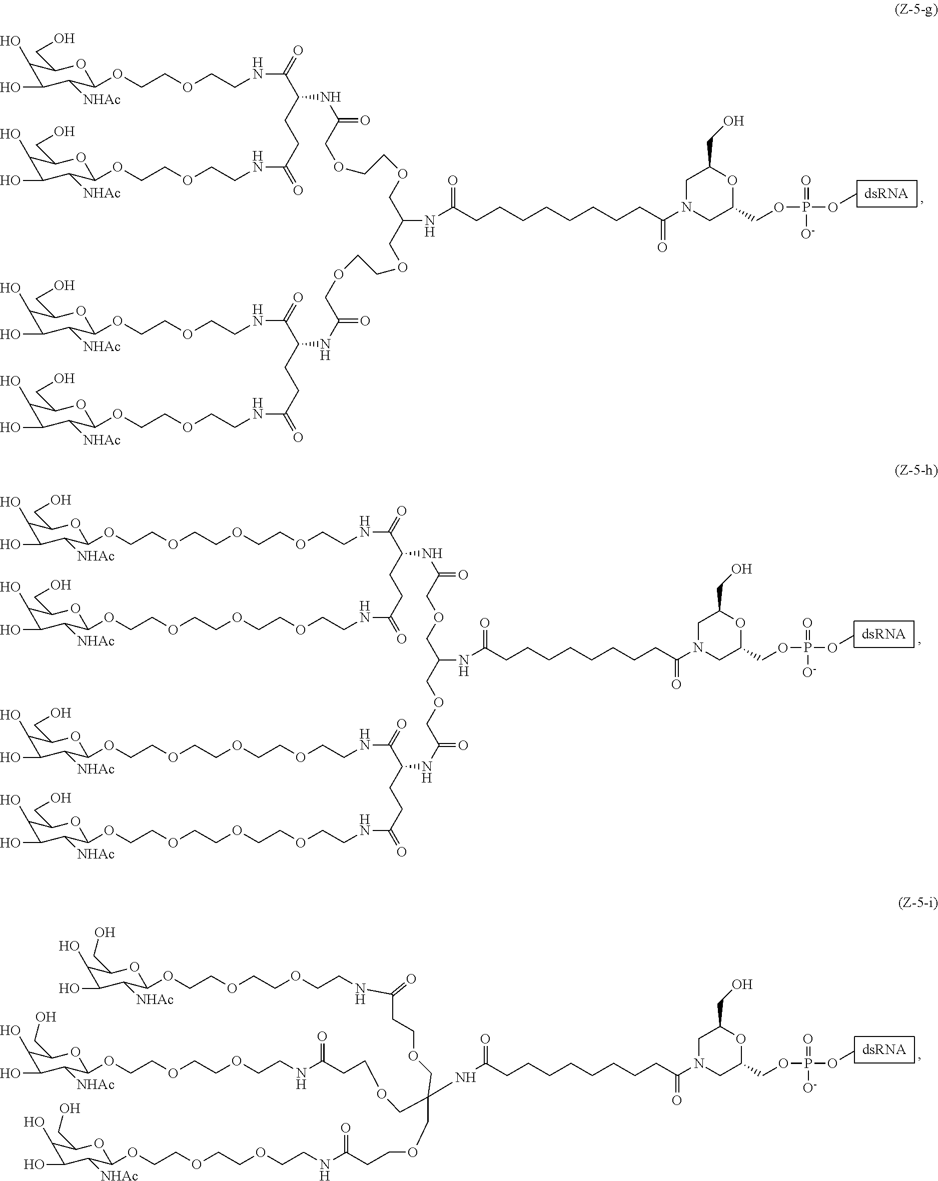

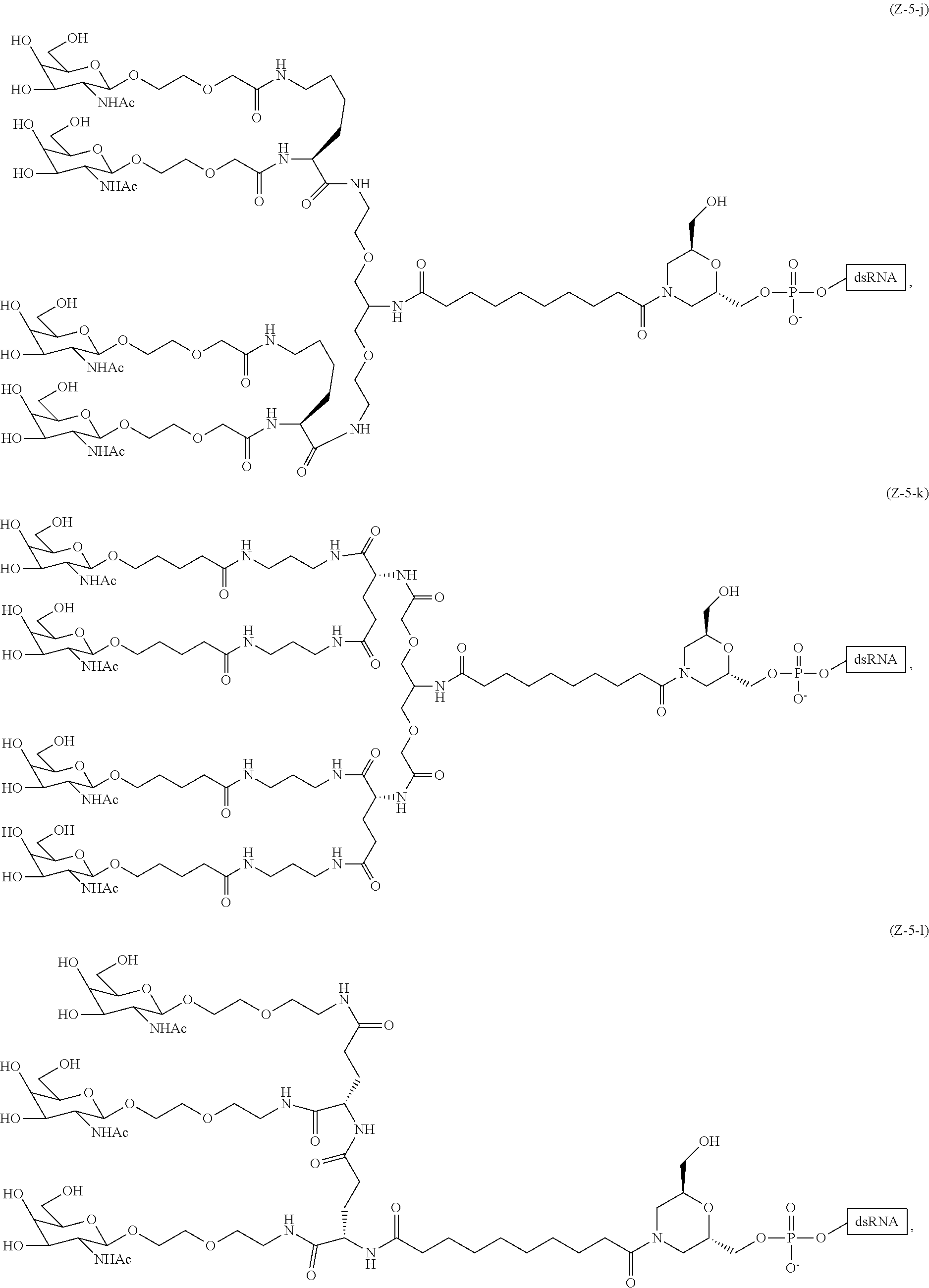

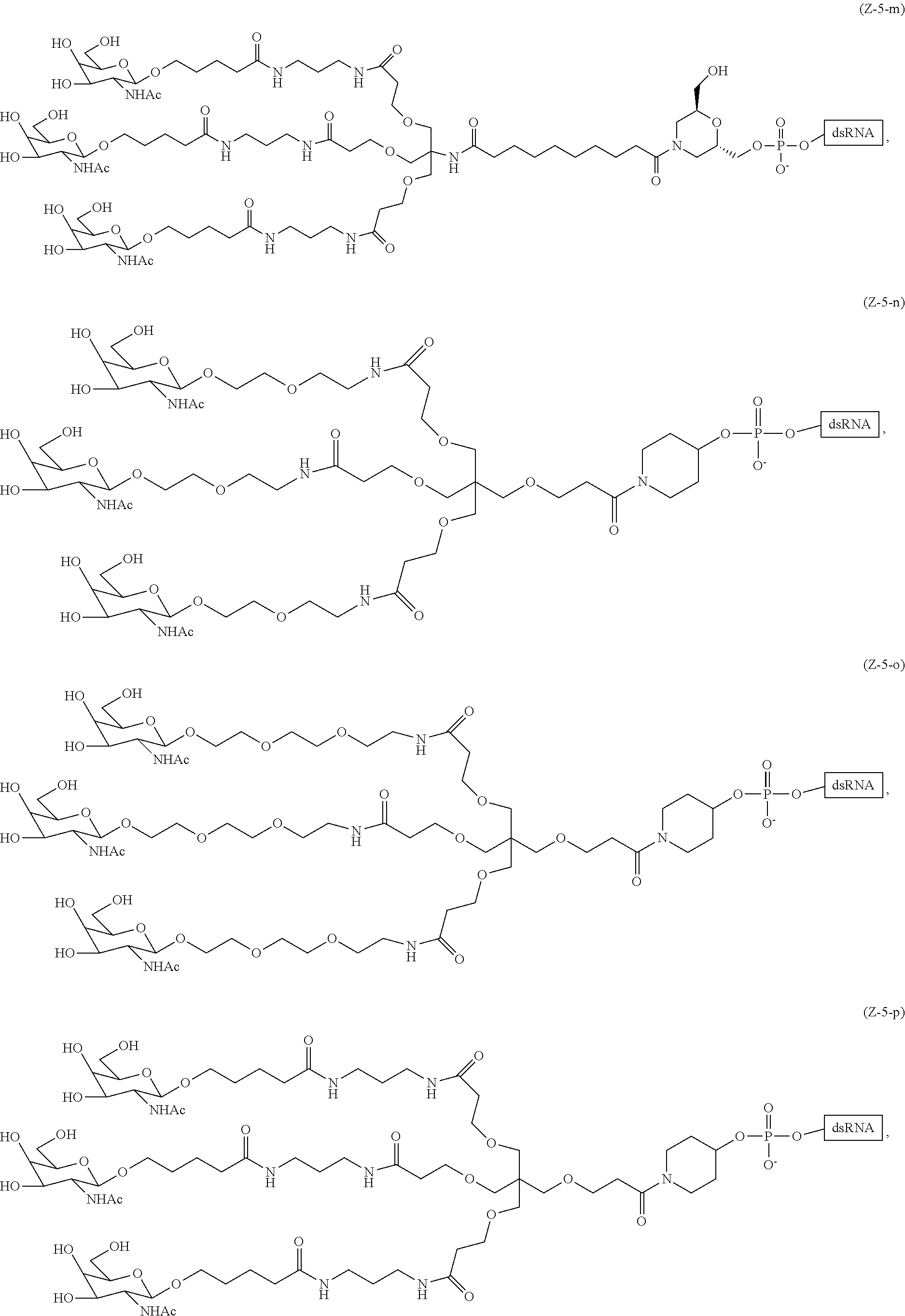

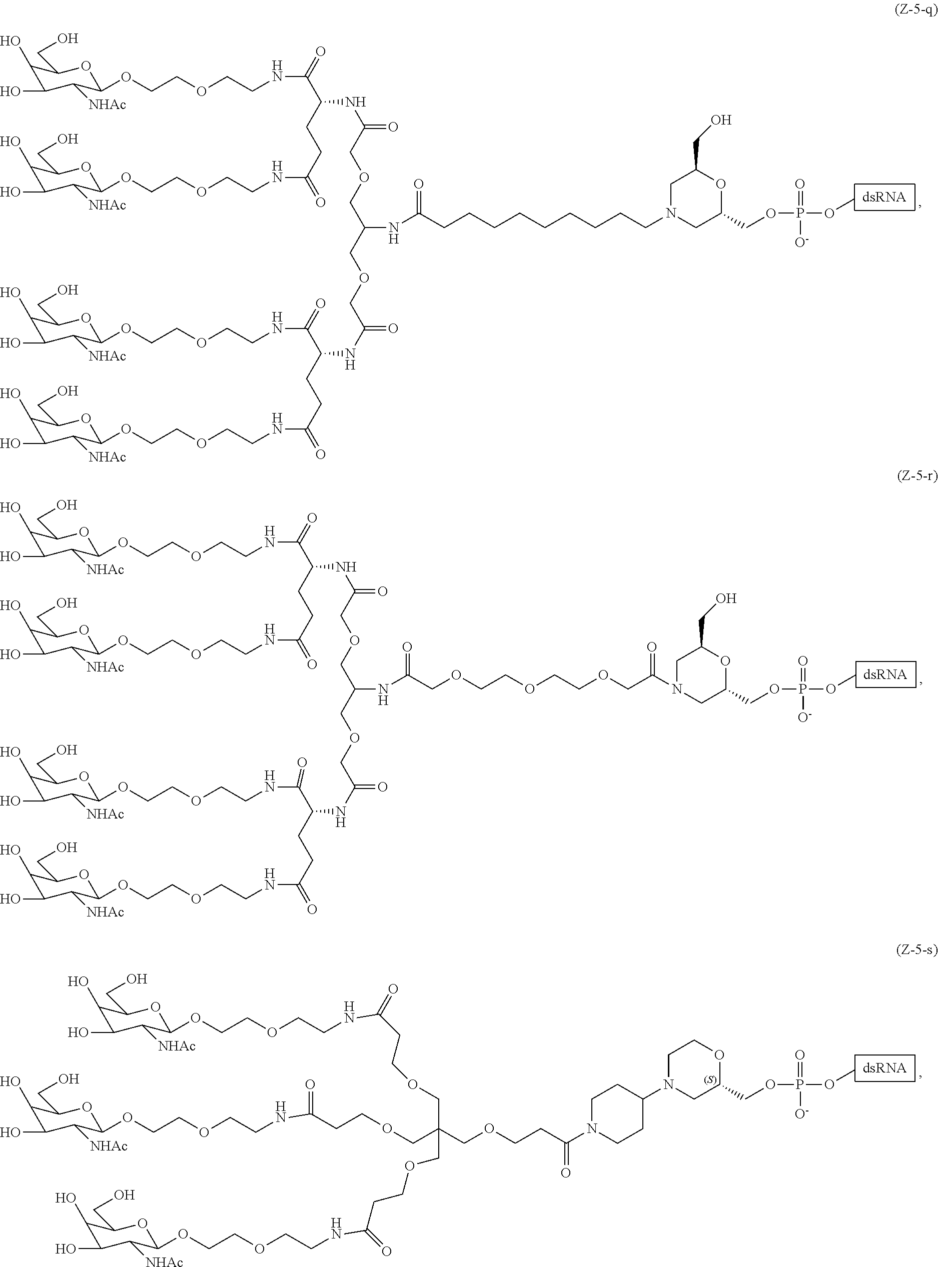

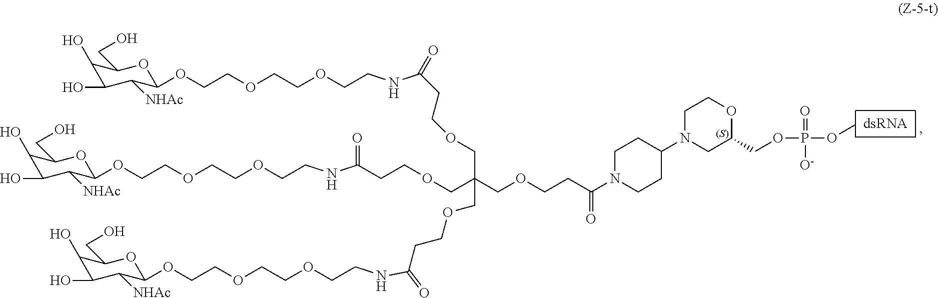

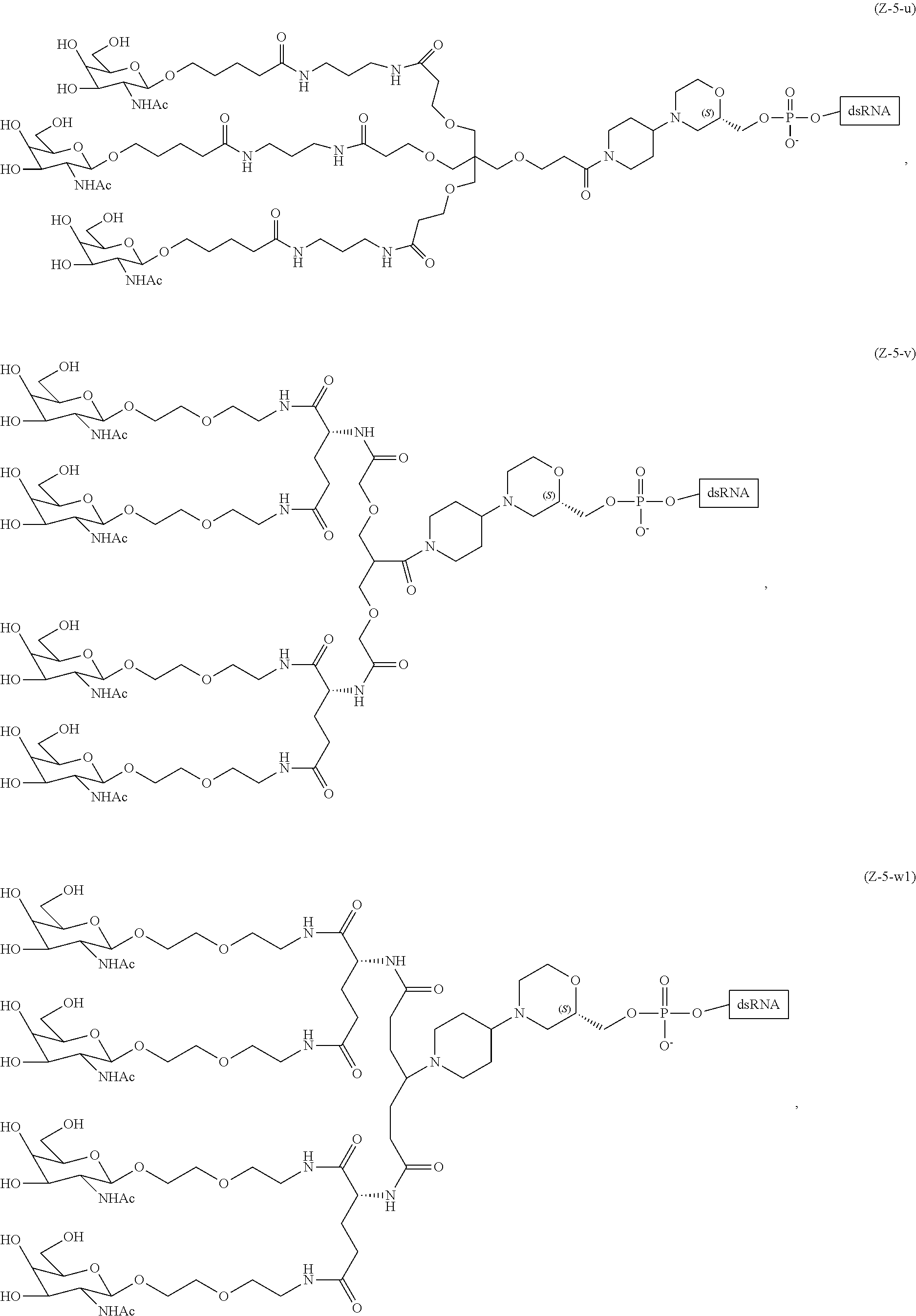

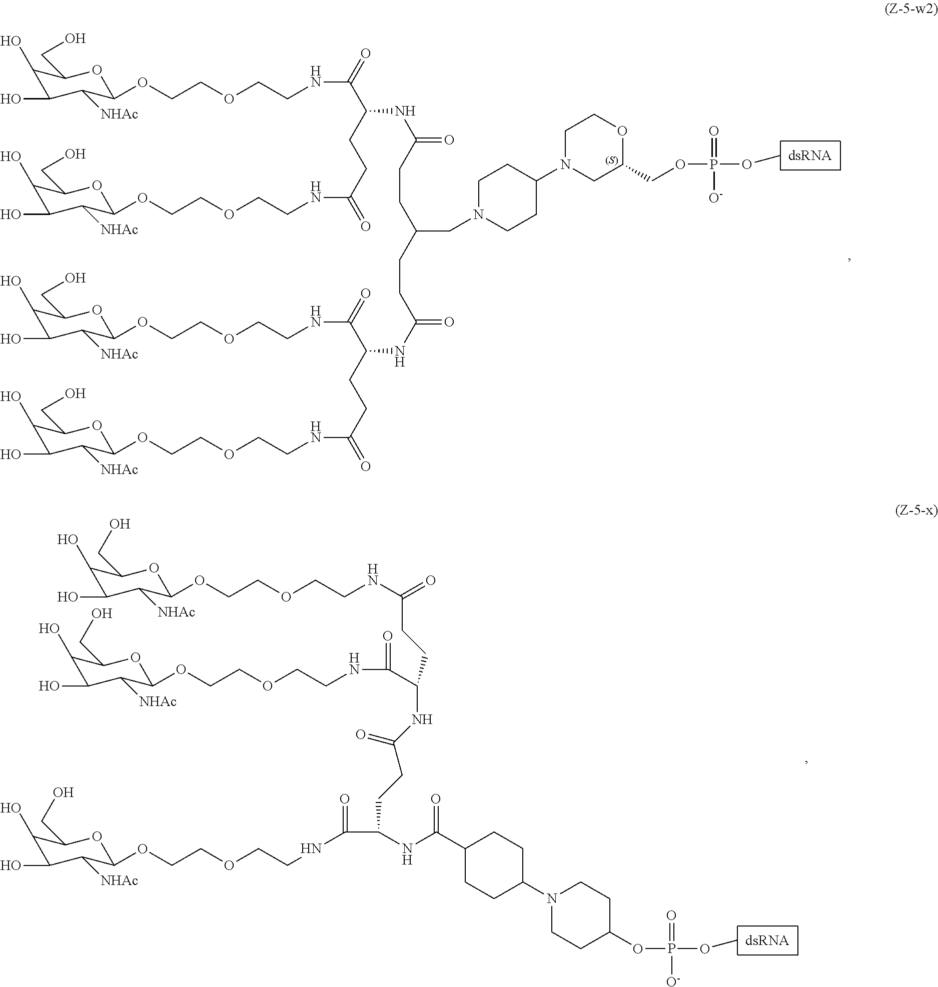

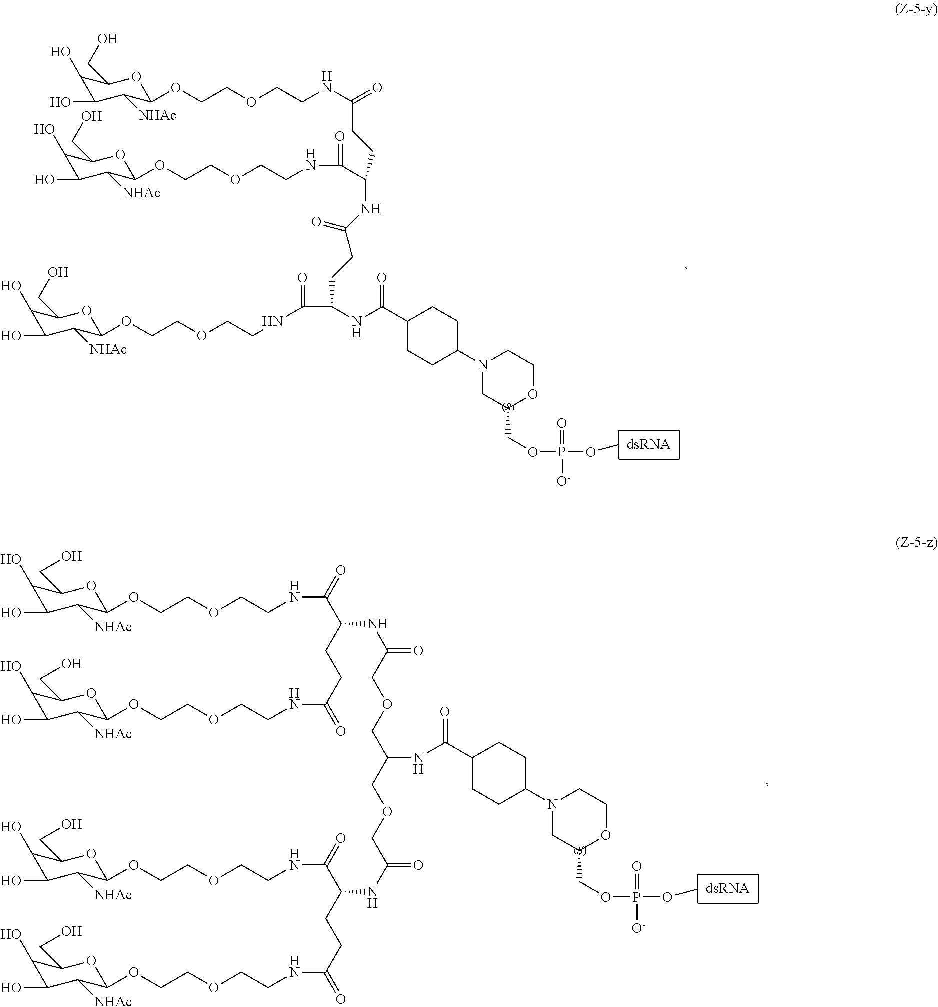

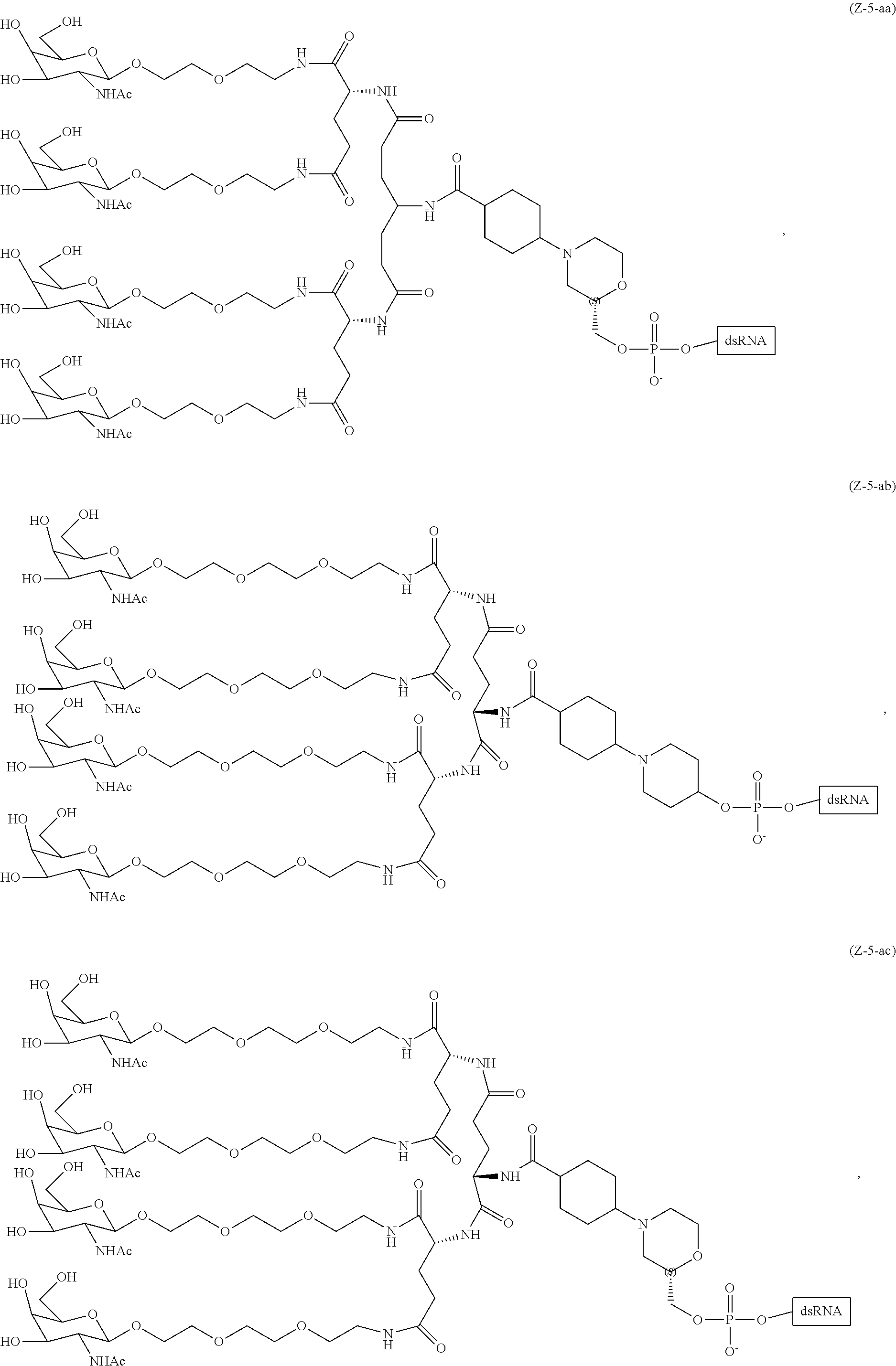

or pharmaceutically acceptable thereof,
wherein the dsRNA includes any one of PCSK9 siRNA in Table 6e, the dsRNA includes:
   (i) a sense strand; and
   (ii) an antisense strand forming a duplex with the sense strand.

TABLE 6e

| PCSK9 | | | SEQ ID |
|---|---|---|---|
| SIRNA | Sequence (5′-3′) | Strand | NO |
| 323 | C004p001U004p001A004pG004pA004pC004pC007pU004pG007 | SS | 2136 |
| | pU004PT002pU004pU004pG004pC004pU004pU004pU004pU004 | | |
| | pG004pU004 | | |
| | A004p001C007p001A004pA007pA007pA007pG004pC007pA004 | AS | 2137 |
| | pA007pA004pA007pC004pA007pG004pG007pU004pC007pU004 | | |
| | pA004pG004p001A004p001A004 | | |
| 324 | C004p001U004p001A004pG004pA004pC004pC007pU004pG007 | SS | 2138 |
| | pU004pT002pU004pU004pG004pC004pU004pU004pU004pU004 | | |
| | pG004p001U004 | | |
| | A004p001C007p001A004pA007pA007pA007pG004pC007pA004 | AS | 2139 |
| | pA007pA004pA007pC004pA007pG004pG007pU004pC007pU004 | | |
| | pA004pG004p001A004p001A004 | | |
| 325 | U004p001G004p001U004pU004pU004pU004pG007pC007pU007 | SS | 2140 |
| | pU004pU004pU004pG004pU004pA004pA004pC004p001U004p0 | | |
| | 01U004 | | |
| | A004p001A007p001G004pU007pU004pA007pC004pA007pA004 | AS | 2141 |
| | pA007pA004pG007pC004pA007pA004pA007pA004pC007pA004 | | |
| | p001G007p001G004 | | |
| 326 | U004p001G004p001U004pU004pU004pU004pG007pC007pU007 | SS | 2142 |
| | pU004pU004pU004pG004pU004pA004pA004pC004pU004p001U | | |
| | 004 | | |
| | A004p001A007p001G004pU007pU004pA007pC004pA007pA004 | AS | 2143 |
| | pA007pA004pG007pC004pA007pA004pA007pA004pC007pA004 | | |
| | p001G007p001G004 | | |
| 327 | G004p001U004p001U004pU004pU004pG004pC007pU007pU007 | SS | 2144 |
| | pU004pU004pG004pU004pA004pA004pC004pU004p001U004p0 | | |
| | 01A004 | | |
| | U004p001A007p001A004pG007pU004pU007pA004pC007pA004 | AS | 2145 |
| | pA007pA004pA007pG004pC007pA004pA007pA004pA007pC004 | | |
| | p001A007p001G004 | | |
| 328 | G004p001U004p001U004pU004pU004pG004pC007pU007pU007 | SS | 2146 |
| | pU004pU004pG004pU004pA004pA004pC004pU004p001U004p0 | | |
| | 01A004 | | |
| | U004p001A007p001A004pG007pU004pU007pA004pC007pA004 | AS | 2147 |
| | pA007pA004pA007pG004pC007pA004pA007pA004pA007pC004 | | |
| | p001A007p001G004 | | |
| 329 | G004p001U004p001U004pU004pU004pG004pC007pU007pU007 | SS | 2148 |
| | pU004pU004pG004pU004pA004pA004pC004pU004pU004p001A | | |
| | 004 | | |
| | U004p001A007p001A004pG007pU004pU007pA004pC007pA004 | AS | 2149 |
| | pA007pA004pA007pG004pC007pA004pA007pA004pA007pC004 | | |
| | p001A007p001G004 | | |
| 330 | G004p001U004p001U004pU004pU004pG004pC007pU007pU007 | SS | 2150 |
| | pU004pU004pG004pU004pA004pA004pC004pU004pU004pA004 | | |
| | U004p001A007p001A004pG007pU004pU007pA004pC007pA004 | AS | 2151 |
| | pA007pA004pA007pG004pC007pA004pA007pA004pA007pC004 | | |
| | p001A007p001G004 | | |
| 331 | U004p001U004p001U004pU004pG004pC004pU007pU007pU007 | SS | 2152 |
| | pU004pG004pU004pA004pA004pC004pU004pU004p001G004p0 | | |
| | 01A004 | | |
| | U004p001C007p001A004pA007pG004pU007pU004pA007pC004 | AS | 2153 |
| | pA007pA004pA007pA004pG007pC004pA007pA004pA007pA004 | | |
| | p001C007p001A004 | | |
| 332 | U004p001U004p001U004pU004pG004pC004pU007pU007pU007 | SS | 2154 |
| | pU004pG004pU004pA004pA004pC004pU004pU004p001G004p0 | | |
| | 01A004 | | |
| | U004p001C007p001A004pA007pG004pU007pU004pA007pC004 | AS | 2155 |
| | pA007pA004pA007pA004pG007pC004pA007pA004pA007pA004 | | |
| | p001C007p001A004 | | |
| 333 | U004p001G004p001C004pU004pU004pU004pU007pG007pU007 | SS | 2156 |
| | pA004pA004pC004pU004pU004pG004pA004pA004p001G004p0 | | |
| | 01A004 | | |
| | U004p001C007p001U004pU007pC004pA007pA004pG007pU004 | AS | 2157 |
| | pU007pA004pC007pA004pA007pA004pA007pG004pC007pA004 | | |
| | p001A007p001A004 | | |
| 334 | G004p001C004p001U004pU004pU004pU004pG007pU007pA007 | SS | 2158 |
| | pA004pC004pU004pU004pG004pA004pA004pG004p001A004p0 | | |
| | 01U004 | | |
| | A004p001U007p001C004pU007pU004pC007pA004pA007pG004 | AS | 2159 |
| | pU007pU004pA007pC004pA007pA004pA007pA004pG007pC004 | | |
| | p001A007p001A004 | | |
| 335 | C004p001U004p001U004pU004pU004pG004pU007pA007pA007 | SS | 2160 |
| | pC004pU004pU004pG004pA004pA004pG004pA004p001U004p0 | | |
| | 01A004 | | |
| | U004p001A007p001U004pC007pU004pU007pC004pA007pA004 | AS | 2161 |
| | pG007pU004pU007pA004pC007pA004pA007pA004pA007pG004 | | |
| | p001C007p001A004 | | |
| 336 | U004p001U004p001U004pU004pG004pU004pA007pA007pC007 | SS | 2162 |
| | pU004pU004pG004pA004pA004pG004pA004pU004p001A004p0 | | |
| | 01U004 | | |
| | A004p001U007p001A004pU007pC004pU007pU004pC007pA004 | AS | 2163 |
| | pA007pG004pU007pU004pA007pC004pA007pA004pA007pA004 | | |
| | p001G007p001C004 | | |

Specific codes in the nucleotide sequences are indicated in above, e.g., Tables A and A-1.

Additional suitable second dsRNAi agent targeting PCSK9 in Table 6e, or variants thereof and synthesis thereof are also described in WO2023/241591, entire contents of which are incorporated herein by reference.

In some embodiment, the second dsRNAi agent may have a structure of

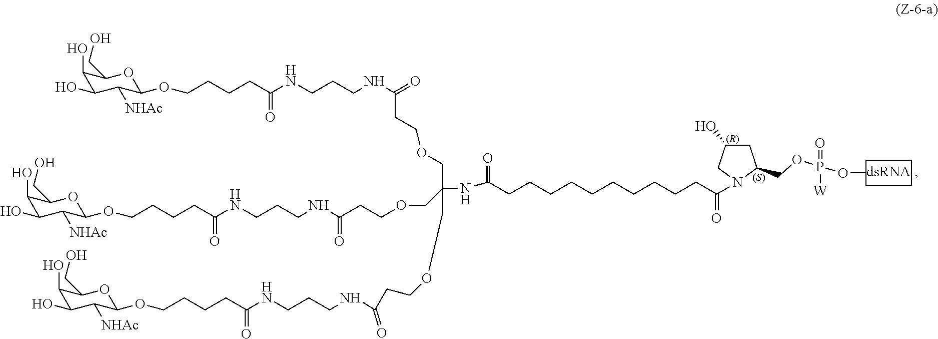

or a pharmaceutically acceptable salt thereof,
wherein W is —SH or —OH, and
the dsRNA includes:
(i) a sense strand including a nucleotide sequence in Table 6f-1; and
(ii) an antisense strand forming a duplex with the sense strand and including a nucleotide sequence in Table 6f-2.

TABLE 6f-1

| | SEQ |
|---|---|
| | ID |
| Sense strand Sequence (5′-3′) | NO |
| IgT3p-IgT3p-IgT3p- | 2164 |
| U007pA004pU007pG004pG007pU004pG007pA004pC007 | |
| pU004pU007pU004pU007pU004pA007pA004pA007p001 | |
| A004p001U007 | |
| IgT3p-IgT3p-IgT3p- | 2165 |
| U004pA004pU004pG004pG007pU004pG007pA007pC007 | |
| pU004pU004pU004pU004pU004pA004pA004pA004 | |
| pA004pU004 | |
| IgT3p-IgT3p-IgT3p- | 2166 |
| U1017pA1017pU004pG004pG007pU004pG007pA007 | |
| pC007pU004pU004pU004pU004pU004pA004pA004 | |
| pA004pA004pU004p-IT4p-IT4 | |
| IgT3p-IgT3p-IgT3p- | 2167 |
| U007pU004pA007pU004pU007pA004pA007pU004pA007 | |
| pU004pG007pG004pU007pG004pA007pC004pU007p001 | |
| U004p001U007 | |
| IgT3p-IgT3p-IgT3p- | 2168 |
| U004pU004pA004pU004pU007pA004pA007pU007pA007 | |
| pU004pG004pG004pU004pG004pA004pC004pU004p001 | |
| U004p001U004 | |
| IgT3p-IgT3p-IgT3p- | 2169 |
| U1017pU1017pA004pU004pU007pA004pA007pU007 | |
| pA007pU004pG004pG004pU004pG004pA004pC004 | |
| pU004p001U004p001U004 | |
| IgT3p-IgT3p-IgT3p- | 2170 |
| U004pU004pA004pU004pU007pA004pA007pU007pA007 | |
| pU004pG004pG004pU004pG004pA004pC004pU004 | |
| pU004pU004p-IT4p-IT4 | |

TABLE 6f-2

| | SEQ |
|---|---|
| | ID |
| Antisense Strand Sequence (5′-3′) | NO |
| A007p001U007p001U004pU007pU004pA007pA004 | 2171 |
| pA007pA004pA007pG004pU007pC004pA007pC004 | |
| pC007pA004pU007pA004p001T002p001T002 | |
| A004p001U007p001U004pU004pU004pA007pA004 | 2172 |
| pA004pA004pA004pG004pU004pC004pA007pC004 | |
| pC007pA004pU004pA004p001A004p001A004 | |
| A007p001A007p001A004pG007pU004pC007pA004 | 2173 |
| pC007pC004pA007pU004pA007pU004pU007pA004 | |
| pA007pU004pA007pA004p001T002p001T002 | |
| A004p001A007p001A004pG007pU004pC004pA004 | 2174 |
| pC004pC004pA004pU004pA004pU004pU007pA004 | |
| pA007pU004pA004pA004p001A004p001A004 | |
| A004p001A007p001A004pG007pU004pC004pA004 | 2175 |
| pC007pC007pA004pU004pA004pU004pU007pA004 | |
| pA007pU004pA004pA004p001A004p001A004 | |
| A004p001A007p001A004pG007pU004pC004pA004 | 2176 |
| pC004pC007pA004pU004pA004pU004pU007pA004 | |
| pA007pU004pA004pA004p001A004p001A004 | |
| A004p001A007p001A004pG007pU004pC004pA004 | 2177 |
| pC007pC007pA004pU004pA004pU004pU007pA004 | |
| pA007pU004pA004pA004p001A1017p001A1017 | |
| A004p001A007p001A004pG007pU004pC004pA004 | 2178 |
| pC004pC007pA004pU004pA004pU004pU007pA004 | |
| pA007pU004pA004pA004p001A1017p001A1017 | |

Specific codes in the nucleotide sequences are indicated in above, e.g., Tables A and A-1.

In some embodiment, the second dsRNAi agent may have a structure of

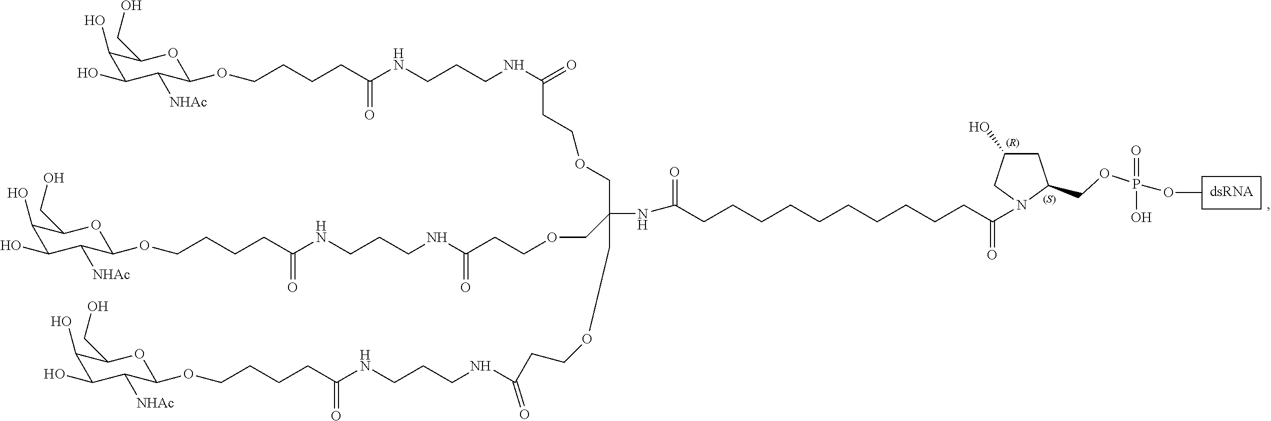

or a pharmaceutically acceptable salt thereof,
wherein the dsRNA includes any one of PCSK9 siRNA in Table 6f-3, the dsRNA includes:
   (i) a sense strand; and
   (ii) an antisense strand forming a duplex with the sense strand.

TABLE 6f-3

| PCSK9 | | | SEQ ID |
|---|---|---|---|
| siRNA | Sequence (5′-3′) | Strand | NO |
| 337 | IgT3pIgT3pIgT3pU007pA004pU007pG004pG007pU004pG007pA | SS | 2179 |
| | 004pC007pU004pU007pU004pU007pU004pA007pA004pA007p00 | | |
| | 1A004p001U007 | | |
| | A007p001U007p001U004pU007pU004pA007pA004pA007pA004p | AS | 2180 |
| | A007pG004pU007pC004pA007pC004pC007pA004pU007pA004p0 | | |
| | 01T002p001T002 | | |
| 338 | IgT3pIgT3pIgT3pU007pU004pA007pU004pU007pA004pA007pU | SS | 2181 |
| | 004pA007pU004pG007pG004pU007pG004pA007pC004pU007p00 | | |
| | 1U004p0010007 | | |
| | A007p001A007p001A004pG007pU004pC007pA004pC007pC004p | AS | 2182 |
| | A007pU004pA007pU004pU007pA004pA007pU004pA007pA004p0 | | |
| | 01T002p001T002 | | |
| 339 | IgT3pIgT3pIgT3pU004pA004pU004pG004pG007pU004pG007pA | SS | 2183 |
| | 007pC007pU004pU004pU004pU004pU004pA004pA004pA004pA0 | | |
| | 04pU004pIT4pIT4 | | |
| | A004p001U007p001U004pU004pU004pA007pA004pA004pA004p | AS | 2184 |
| | A004pG004pU004pC004pA007pC004pC007pA004pU004pA004p0 | | |
| | 01A004p001A004 | | |
| 340 | IgT3pIgT3pIgT3pU1017pA1017pU004pG004pG007pU004pG007 | SS | 2185 |
| | pA007pC007pU004pU004pU004pU004pU004pA004pA004pA004p | | |
| | A004pU004pIT4pIT4 | | |
| | A004p001U007p001U004pU004pU004pA007pA004pA004pA004p | AS | 2186 |
| | A004pG004pU004pC004pA007pC004pC007pA004pU004pA004p0 | | |
| | 01A004p001A004 | | |
| 341 | IgT3pIgT3pIgT3pU004pU004pA004pU004pU007pA004pA007pU | SS | 2187 |
| | 007pA007pU004pG004pG004pU004pG004pA004pC004pU004p00 | | |
| | 1U004p001U004 | | |
| | A004p001A007p001A004pG007pU004pC004pA004pC007pC007p | AS | 2188 |
| | A004pU004pA004pU004pU007pA004pA007pU004pA004pA004p0 | | |
| | 01A004p001A004 | | |
| 342 | IgT3pIgT3pIgT3pU1017pU1017pA004pU004pU007pA004pA007 | SS | 2189 |
| | pU007pA007pU004pG004pG004pU004pG004pA004pC004pU004p | | |
| | 001U004p001U004 | | |
| | A004p001A007p001A004pG007pU004pC004pA004pC004pC004p | AS | 2190 |
| | A004pU004pA004pU004pU007pA004pA007pU004pA004pA004p0 | | |
| | 01A004p001A004 | | |
| 343 | IgT3pIgT3pIgT3pU1017pU1017pA004pU004pU007pA004pA007 | SS | 2191 |
| | pU007pA007pU004pG004pG004pU004pG004pA004pC004pU004p | | |
| | 001U004p001U004 | | |
| | A004p001A007p001A004pG007pU004pC004pA004pC007pC007p | AS | 2192 |
| | A004pU004pA004pU004pU007pA004pA007pU004pA004pA004p0 | | |
| | 01A004p001A004 | | |
| 344 | IgT3pIgT3pIgT3pU1017pA004pU004pU007pA004pA007pU007p | SS | 2193 |
| | A007pU004pG004pG004pU004pG004pA004pC004pU004p001U00 | | |
| | 4p001U004 | | |
| | A004p001A007p001A004pG007pU004pC004pA004pC004pC007p | AS | 2194 |
| | A004pU004pA004pU004pU007pA004pA007pU004pA004pA004p0 | | |
| | 01A004p001A004 | | |
| 345 | IgT3pIgT3pIgT3pU004pU004pA004pU004pU007pA004pA007pU | SS | 2195 |
| | 007pA007pU004pG004pG004pU004pG004pA004pC004pU004pU0 | | |
| | 04pU004pIT4pIT4 | | |
| | A004p001A007p001A004pG007pU004pC004pA004pC007pC007p | AS | 2196 |
| | A004pU004pA004pU004pU007pA004pA007pU004pA004pA004p0 | | |
| | 01A1017p001A1017 | | |
| 346 | IgT3pIgT3pIgT3pU004pU004pA004pU004pU007pA004pA007pU | SS | 2197 |
| | 007pA007pU004pG004pG004pU004pG004pA004pC004pU004pU0 | | |
| | 04pU004pIT4pIT4 | | |
| | A004p001A007p001A004pG007pU004pC004pA004pC004pC007p | AS | 2198 |
| | A004pU004pA004pU004pU007pA004pA007pU004pA004pA004p0 | | |
| | 01A1017p001A1017 | | |

IgT3: 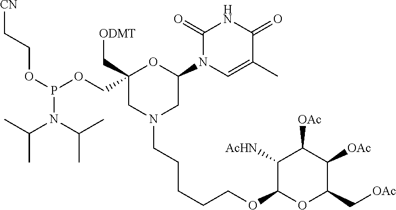
IT4:

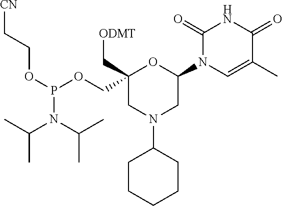

Specific codes in the nucleotide sequences are indicated in above, e.g., Tables A and A-1.

Additional suitable second dsRNAi agent targeting PCSK9 according to Tables 6f-1 to 3, or variants thereof and synthesis thereof are also described in WO2021/037972, entire contents of which are incorporated herein by reference.

In some embodiment, the second dsRNAi agent may have a structure of

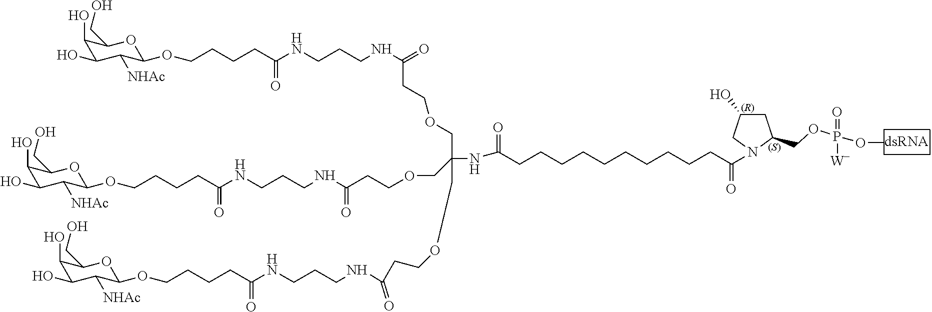

wherein W is S or O and the dsRNA includes any one of PCSK9 siRNA in Table 6f-3, the dsRNA includes:
   (i) a sense strand; and
   (ii) an antisense strand forming a duplex with the sense strand.

TABLE 6g

| PCSK9 | | | SEQ ID |
|---|---|---|---|
| SIRNA | Sequence (5′-3′) | Strand | NO |
| 347 | A004p001G004p001A004pA004pU004pG004pA007pC004pU007p | SS | 2199 |
| | U004pU004pU004pA004pU004pU004pG004pA004pG004pC004pU | | |
| | 004pC004 | | |
| | A004p001A007p001G004pA007pG007pC007pU004pC007pA004p | AS | 2200 |
| | A007pU004pA007pA004pA007pA004pG007pU004pC007pA004p0 | | |
| | 01U004p001U004 | | |
| 348 | A004p001U004p001U004pU004pC004pA004pC007pC004pA007p | SS | 2201 |
| | U004pU004pC004pA004pA004pA004pC004pA004pG004pG004pU | | |
| | 004pC004 | | |
| | U004p001C007p001G004pA007pC007pC007pU004pG007pU004p | AS | 2202 |
| | U007pU004pG007pA004pA007pU004pG007pG004pU007pG004p0 | | |
| | 01A004p001A004 | | |
| 349 | C004p001G004p001A004pU004pG004pU004pC007pC004pG007p | SS | 2203 |
| | U004pG004pG004pG004pC004pA004pG004pA004pA004pU004pG | | |
| | 004pA004 | | |
| | A004p001G007p001U004pC007pA007pU007pU004pC007pU004p | AS | 2204 |
| | G007pC004pC007pC004pA007pC004pG007pG004pA007pC004p0 | | |
| | 01A004p001U004 | | |
| 350 | U004p001U004p001A004pU004pU004pG004pA007pG004pC007p | SS | 2205 |
| | U004pC004pU004pU004pG004pU004pU004pC004pC004pG004pU | | |
| | 004pG004 | | |
| | G004p001G007p001C004pA007pC007pG007pG004pA007pA004p | AS | 2206 |
| | C007pA004pA007pG004pA007pG004pC007pU004pC007pA004p0 | | |
| | 01A004p001U004 | | |
| 351 | G004p001C004p001U004pC004pC004pC004pA007pA004pU007p | SS | 2207 |
| | G004pU004pG004pC004pC004pG004pA004pU004pG004pU004pC | | |
| | 004pC004 | | |
| | A004p001C007p001G004pG007pA007pC007pA004pU007pC004p | AS | 2208 |
| | G007pG004pC007pA004pC007pA004pU007pU004pG007pG004p0 | | |
| | 01G004p001A004 | | |
| 352 | U004p001G004p001C004pC004pG004pA004pU007pG004pU007p | SS | 2209 |
| | C004pC004pG004pU004pG004pG004pG004pC004pA004pG004pA | | |
| | 004pA004 | | |
| | C004p001A007p001U004pU007pC007pU007pG004pC007pC004p | AS | 2210 |
| | C007pA004pC007pG004pG007pA004pC007pA004pU007pC004p0 | | |
| | 01G004p001G004 | | |
| 353 | C004p001A004p001C004pC004pA004pU004pU007pC004pA007p | SS | 2211 |
| | A004pA004pC004pA004pG004pG004pU004pC004pG004pA004pG | | |
| | 004pC004 | | |
| | C004p001A007p001G004pC007pU007pC007pG004pA007pC004p | AS | 2212 |
| | C007pU004pG007pU004pU007pU004pG007pA004pA007pU004p0 | | |
| | 01G004p001G004 | | |
| 354 | C004p001C004p001A004pA004pU004pG004pU007pG004pC007p | SS | 2213 |
| | C004pG004pA004pU004pG004pU004pC004pC004pG004pU004pG | | |
| | 004pG004 | | |
| | G004p001C007p001C004pC007pA007pC007pG004pG007pA004p | AS | 2214 |
| | C007pA004pU007pC004pG007pG004pC007pA004pC007pA004p0 | | |
| | 01U004p001U004 | | |
| 355 | U004p001U004p001U004pA004pU004pU004pG007pA004pG007p | SS | 2215 |
| | C004pU004pC004pU004pU004pG004pU004pU004pC004pC004pG | | |
| | 004pU004 | | |
| | G004p001C007p001A004pC007pG007pG007pA004pA007pC004p | AS | 2216 |
| | A007pA004pG007pA004pG007pC004pU007pC004pA007pA004p0 | | |
| | 01U004p001A004 | | |
| 356 | G004p001G004p001G004pC004pU004pG004pA007pG004pC007p | SS | 2217 |
| | U004pU004pU004pA004pA004pA004pA004pU004pG004pG004pU | | |
| | 004pU004 | | |
| | G004p001G007p001A004pA007pC007pC007pA004pU007pU004p | AS | 2218 |
| | U007pU004pA007pA004pA007pG004pC007pU004pC007pA004p0 | | |
| | 01G004p001C004 | | |
| 357 | G004p001G004p001C004pA004pU004pU004pU007pC004pA007p | SS | 2219 |
| | C004pC004pA004pU004pU004pC004pA004pA004pA004pC004pA | | |
| | 004pG004 | | |
| | A004p001C007p001C004pU007pG007pU007pU004pU007pG004p | AS | 2220 |
| | A007pA004pU007pG004pG007pU004pG007pA004pA007pA004p0 | | |
| | 01U004p001G004 | | |
| 358 | C004p001A004p001U004pU004pU004pC004pA007pC004pC007p | SS | 2221 |
| | A004pU004pU004pC004pA004pA004pA004pC004pA004pG004pG | | |
| | 004pU004 | | |
| | C004p001G007p001A004pC007pC007pU007pG004pU007pU004p | AS | 2222 |
| | U007pG004pA007pA004pU007pG004pG007pU004pG007pA004p0 | | |
| | 01A004p001A004 | | |
| 359 | G007p001C004p001A007pU004pU007pU004pC007pA004pC007p | SS | 2223 |
| | C007pA1017pU004pU007pC004pA007pA004pA007pC004pA007p | | |
| | G004pG007 | | |
| | C004p001C007p001U004pG007pU004pU007pU004pG007pA004p | AS | 2224 |
| | A007pU004pG004pG004pU007pG004pA007pA004pA007pU004pG | | |
| | 007pC004p001C007p001C004 | | |
| 360 | G007p001C004p001A007pU004pU007pU004pC007pA004pC007p | SS | 2225 |
| | C007pA007pU004pU007pC004pA007pA004pA007pC004pA007pG | | |
| | 004pG007 | | |
| | C004p001C007p001U004pG007pU004pU007pU004pG007pA004p | AS | 2226 |
| | A007pU004pG004pG004pU007pG004pA007pA004pA007pU004pG | | |
| | 007pC004p001C004p001C004 | | |
| 361 | A007p001A004p001U007pG004pA007pC004pU007pU004pU007p | SS | 2227 |
| | U007pA1017pU004pU007pG004pA007pG004pC007pU004pC007p | | |
| | U004pU007 | | |
| | A004p001A007p001G004pA007pG004pC007pU004pC007pA004p | AS | 2228 |
| | A007pU004pA004pA004pA007pA004pG007pU004pC007pA004pU | | |
| | 007pU004p001C007p001U004 | | |
| 362 | A007p001A004p001U007pG004pA007pC004pU007pU004pU007p | SS | 2229 |
| | U007pA007pU004pU007pG004pA007pG004pC007pU004pC007pU | | |
| | 004pU007 | | |
| | A004p001A007p001G004pA007pG004pC007pU004pC007pA004p | AS | 2230 |
| | A007pU004pA004pA004pA007pA004pG007pU004pC007pA004pU | | |
| | 007pU004p001C004p001U004 | | |

Specific codes in the nucleotide sequences are indicated in above, e.g., Tables A and A-1.

Additional suitable second dsRNAi agent targeting PCSK9 in Table 6g, or variants thereof and synthesis thereof are also described in WO2022/089486, entire contents of which are incorporated herein by reference.

In some embodiment, the second dsRNAi agent may have a structure of

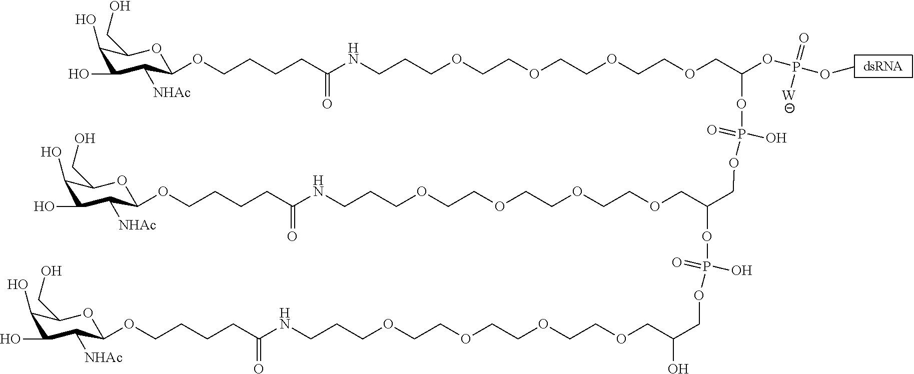

wherein W is S or O and the dsRNA includes:
(i) a sense strand including a nucleotide sequence in Table 6h-1; and
(ii) an antisense strand forming a duplex with the sense strand and including a nucleotide sequence in Table 6h-2.

TABLE 6h-1

| Sense Strand Sequence (5′-3′) | SEQ ID NO |
|---|---|
| U004p001U007p001G007pU007pA004pA004pC004pU004pU004pG004pA004pA00 | 2231 |
| 4p001G004p001A004 | |
| C004p001U007p001G007pU007pU004pU004pU004pG004pC004pU004pU004pU00 | 2232 |
| 4p001U004p001A004 | |
| G004p001U007p001U007pU007pU004pG004pC004pU004pU004pU004pU004pG00 | 2233 |
| 4p001U004p001A004 | |
| A004p001G007p001A007pC007pC004pU004pG004pU004pU004pU004pU004pG00 | 2234 |
| 4p001C004p001A004 | |
| U004p001G007p001U007pU007pU004pU004pG004pC004pU004pU004pU004pU00 | 2235 |
| 4p001G004p001A004 | |
| A004p001G007p001A007pU007pA004pU004pU004pU004pA004pU004pU004pC00 | 2236 |
| 4p001U004p001A004 | |
| C004p001C007p001U007pG007pU004pU004pU004pU004pG004pC004pU004pU00 | 2237 |
| 4p001U004p001A004 | |
| U004p001G007p001U007pA007pA004pC004pU004pU004pG004pA004pA004pG00 | 2238 |
| 4p001A004p001A004 | |
| C004p001U007p001U007pU007pU004pG004pU004pA004pA004pC004pU004pU00 | 2239 |
| 4p001G004p001A004 | |
| U004p001A007p001G007pA007pC004pC004pU004pG004pU004pU004pU004pU00 | 2240 |
| 4p001G004p001A004 | |
| U004p001G007p001A007pA007pG004pA004pU004pA004pU004pU004pU004pA00 | 2241 |
| 4p001U004p001A004 | |
| U004p001U007p001U007pG007pC004pU004pU004pU004pU004pG004pU004pA00 | 2242 |
| 4p001A004p001A004 | |
| U004p001G007p001C007pU007pU004pU004pG004pU004pG004pU004pC004pA00 | 2243 |
| 4p001C004p001A004 | |
| U004p001U007p001U007pG007pU004pA004pA004pC004pU004pU004pG004pA00 | 2244 |
| 4p001A004p001A004 | |
| C004p001U007p001U007pG007pA004pA004pG004pA004pU004pA004pU004pU00 | 2245 |
| 4p001U004p001A004 | |
| A004p001G007p001C007pA007pG004pA004pC004pA004pU004pU004pU004pA00 | 2246 |
| 4p001U004p001A004 | |
| G004p001G007p001A007pG007pU004pU004pU004pA004pU004pU004pC004pG00 | 2247 |
| 4p001G004p001A004 | |
| U004p001U007p001A007pA007pA004pA004pU004pG004pG004pU004pU004pC00 | 2248 |
| 4p001C004p001A004 | |
| A004p001U007p001A007pU007pU004pU004pA004pU004pU004pC004pU004pG00 | 2249 |
| 4p001G004p001A004 | |
| A004p001A007p001C007pU007pU004pG004pA004pA004pG004pA004pU004pA00 | 2250 |
| 4p001U004p001A004 | |
| A004p001G007p001C007pA007pG004pG004pA004pA004pC004pU004pG004pA00 | 2251 |
| 4p001G004p001A004 | |
| C004p001U007p001G007pG007pG004pU004pU004pU004pU004pG004pU004pA00 | 2252 |
| 4p001G004p001A004 | |
| C004p001A007p001U007pU007pU004pA004pU004pC004pU004pU004pU004pU00 | 2253 |
| 4p001G004p001A004 | |
| A004p001G007p001U007pU007pU004pA004pU004pU004pC004pG004pG004pA00 | 2254 |
| 4p001A004p001A004 | |
| G004p001G007p001A007pA007pC004pU004pG004pA004pG004pC004pC004pA00 | 2255 |
| 4p001G004p001A004 | |
| A004p001U007p001G007pA007pU004pG004pC004pU004pG004pU004pC004pU00 | 2256 |
| 4p001G004p001A004 | |
| A004p001G007p001C007pA007pU004pG004pG004pA004pA004pU004pC004pC00 | 2257 |
| 4p001C004p001A004 | |

TABLE 6h-2

| Antisense Strand Sequence (5′-3′) | SEQ ID NO |
|---|---|
| U004p001C004p001U004pU004pC004pA004pA004pG004pU004pU004pA004pC00 | 2258 |
| 4pA004pA007p001A004p001A004p001G004p001C004p001A000 | |
| U004p001A004p001A004pA004pA004pG004pC004pA004pA004pA004pA004pC00 | 2259 |
| 4pA004pG007p001G004p001U004p001C004p001U004p001A000 | |
| U004p001A004p001C004pA004pA004pA004pA004pG004pC004pA004pA004pA00 | 2260 |
| 4pA004pC007p001A004p001G004p001G004p001U004p001C000 | |
| U004p001G004p001C004pA004pA004pA004pA004pC004pA004pG004pG004pU00 | 2261 |
| 4pC004pU007p001A004p001G004p001A004p001A004p001A000 | |
| U004p001C004p001A004pA004pA004pA004pG004pC004pA004pA004pA004pA00 | 2262 |
| 4pC004pA007p001G004p001G004p001U004p001C004p0010000 | |
| U004p001A004p001G004pA004pA004pU004pA004pA004pA004pU004pA004pU00 | 2263 |
| 4pC004pU007p001U004p001C004p001A004p001A004p001G000 | |
| U004p001A004p001A004pA004pG004pC004pA004pA004pA004pA004pC004pA00 | 2264 |
| 4pG004pG007p001U004p001C004p001U004p001A004p001G000 | |
| U004p001U004p001C004pU004pU004pC004pA004pA004pG004pU004pU004pA00 | 2265 |
| 4pC004pA007p001A004p001A004p001A004p001G004p001C000 | |
| U004p001C004p001A004pA004pG004pU004pU004pA004pC004pA004pA004pA00 | 2266 |
| 4pA004pG007p001C004p001A004p001A004p001A004p001A000 | |
| U004p001C004p001A004pA004pA004pA004pC004pA004pG004pG004pU004pC00 | 2267 |
| 4pU004pA007p001G004p001A004p001A004p001A004p001A000 | |
| U004p001A004p001U004pA004pA004pA004pU004pA004pU004pC004pU004pU00 | 2268 |
| 4pC004pA007p001A004p001G004p001U004p001U004p001A000 | |
| U004p001U004p001U004pA004pC004pA004pA004pA004pA004pG004pC004pA00 | 2269 |
| 4pA004pA007p001A004p001C004p001A004p001G004p001G000 | |
| U004p001G004p001U004pG004pA004pC004pA004pC004pA004pA004pA004pG00 | 2270 |
| 4pC004pA007p001G004p001G004p001U004p001G004p001C000 | |
| U004p001U004p001U004pC004pA004pA004pG004pU004pU004pA004pC004pA00 | 2271 |
| 4pA004pA007p001A004p001G004p001C004p001A004p001A000 | |
| U004p001A004p001A004pA004pU004pA004pU004pC004pU004pU004pC004pA00 | 2272 |
| 4pA004pG007p001U004p001U004p001A004p001C004p001A000 | |
| U004p001A004p001U004pA004pA004pA004pU004pG004pU004pC004pU004pG00 | 2273 |
| 4pC004pU007p001U004p001G004p001C004p001U004p001U000 | |
| U004p001C004p001C004pG004pA004pA004pU004pA004pA004pA004pC004pU00 | 2274 |
| 4pC004pC007p001A004p001G004p001G004p001C004p001C000 | |
| U004p001G004p001G004pA004pA004pC004pC004pA004pU004pU004pU004pU00 | 2275 |
| 4pA004pA007p001A004p001G004p001C004p001U004p001C000 | |
| U004p001C004p001C004pA004pG004pA004pA004pU004pA004pA004pA004pU00 | 2276 |
| 4pA004pU007p001C004p001U004p001U004p001C004p001A000 | |
| U004p001A004p001U004pA004pU004pC004pU004pU004pC004pA004pA004pG00 | 2277 |
| 4pU004pU007p001A004p001C004p001A004p001A004p001A000 | |
| U004p001C004p001U004pC004pA004pG004pU004pU004pC004pC004pU004pG00 | 2278 |
| 4pC004pU007p001G004p001U004p001G004p001U004p001G000 | |
| U004p001C004p001U004pA004pC004pA004pA004pA004pA004pC004pC004pC00 | 2279 |
| 4pA004pG007p001A004p001A004p001U004p001A004p001A000 | |
| U004p001C004p001A004pA004pA004pA004pG004pA004pU004pA004pA004pA00 | 2280 |
| 4pU004pG007p001U004p001C004p001U004p001G004p001C000 | |
| U004p001U004p001U004pC004pC004pG004pA004pA004pU004pA004pA004pA00 | 2281 |
| 4pC004pU007p001C004p001C004p001A004p001G004p001G000 | |
| U004p001C004p001U004pG004pG004pC004pU004pC004pA004pG004pU004pU00 | 2282 |
| 4pC004pC007p001U004p001G004p001C004p001U004p001G000 | |
| U004p001C004p001A004pG004pA004pC004pA004pG004pC004pA004pU004pC00 | 2283 |
| 4pA004pU007p001G004p001G004p001C004p001U004p001G000 | |
| U004p001G004p001G004pG004pA004pU004pU004pC004pC004pA004pU004pG00 | 2284 |
| 4pC004pU007p001C004p001C004p001U004p001U004p001G000 | |

Specific codes in the nucleotide sequences are indicated in above, e.g., Tables A and A-1.

In some embodiment, the second dsRNAi agent includes a RNAi agent having a structure of

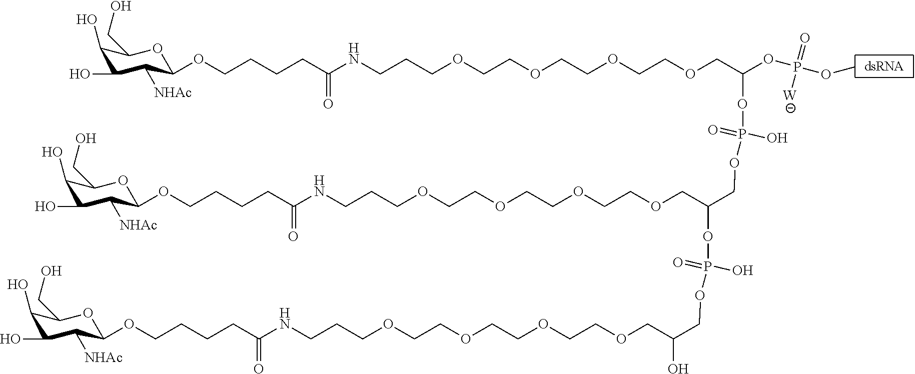

wherein the dsRNA includes any one of PCSK9 siRNA in Table 6h-3, the dsRNA includes:
   (i) a sense strand; and
   (ii) an antisense strand forming a duplex with the sense strand.

TABLE 6h-3

| PCSK9 | | | SEQ ID |
|---|---|---|---|
| siRNA | Sequence (5′-3′) | Strand | NO |
| 363 | A004p001G007p001A004pC007pC004pU007pG004pU007pU004p | SS | 2285 |
| | U007pU004pG007p001C004p001A007 | | |
| | U004p001G007p001C004pA007pA004pA007pA004pC007pA004p | AS | 2286 |
| | G007pG004pU007p001C004p001U007p001A004p001G007p001A | | |
| | 004p001A007p001A000 | | |
| 364 | U004p001G007p001U004pU007pU004pU007pG004pC007pU004p | SS | 2287 |
| | U007pU004pU007p001G004p001A007 | | |
| | U004p001C007p001A004pA007pA004pA007pG004pC007pA004p | AS | 2288 |
| | A007pA004pA007p001C004p001A007p001G004p001G007p001U | | |
| | 004p001C007p0010000 | | |
| 365 | G004p001U007p001U004pU007pU004pG007pC004pU007pU004p | SS | 2289 |
| | U007pU004pG007p001U004p001A007 | | |
| | U004p001A007p001C004pA007pA004pA007pA004pG007pC004p | AS | 2290 |
| | A007pA004pA007p001A004p001C007p001A004p001G007p001G | | |
| | 004p001U007p001C000 | | |
| 366 | U004p001U007p001G004pU007pA004pA007pC004pU007pU004p | SS | 2291 |
| | G007pA004pA007p001G004p001A007 | | |
| | U004p001C007p001U004pU007pC004pA007pA004pG007pU004p | AS | 2292 |
| | U007pA004pC007p001A004p001A007p001A004p001A007p001G | | |
| | 004p001C007p001A000 | | |
| 367 | U007p001A004p001G007pA004pC007pC004pU007pG004pU007p | SS | 2293 |
| | U004pU007pU004pG004p001C004p001A004 | | |
| | U004p001G007p001C004pA007pA004pA007pA004pC007pA004p | AS | 2294 |
| | G007pG004pU007pC004pU007pA004p001G007p001A004p001A0 | | |
| | 07p001A000 | | |

Specific codes in the nucleotide sequences are indicated in above, e.g., Tables A and A-1.

Additional suitable second dsRNAi agent targeting PCSK9 in Tables 6h-1 to 3, or variants thereof and synthesis thereof are also described in WO2022/266486, entire contents of which are incorporated herein by reference.

In some embodiment, the second dsRNAi agent may have a structure of

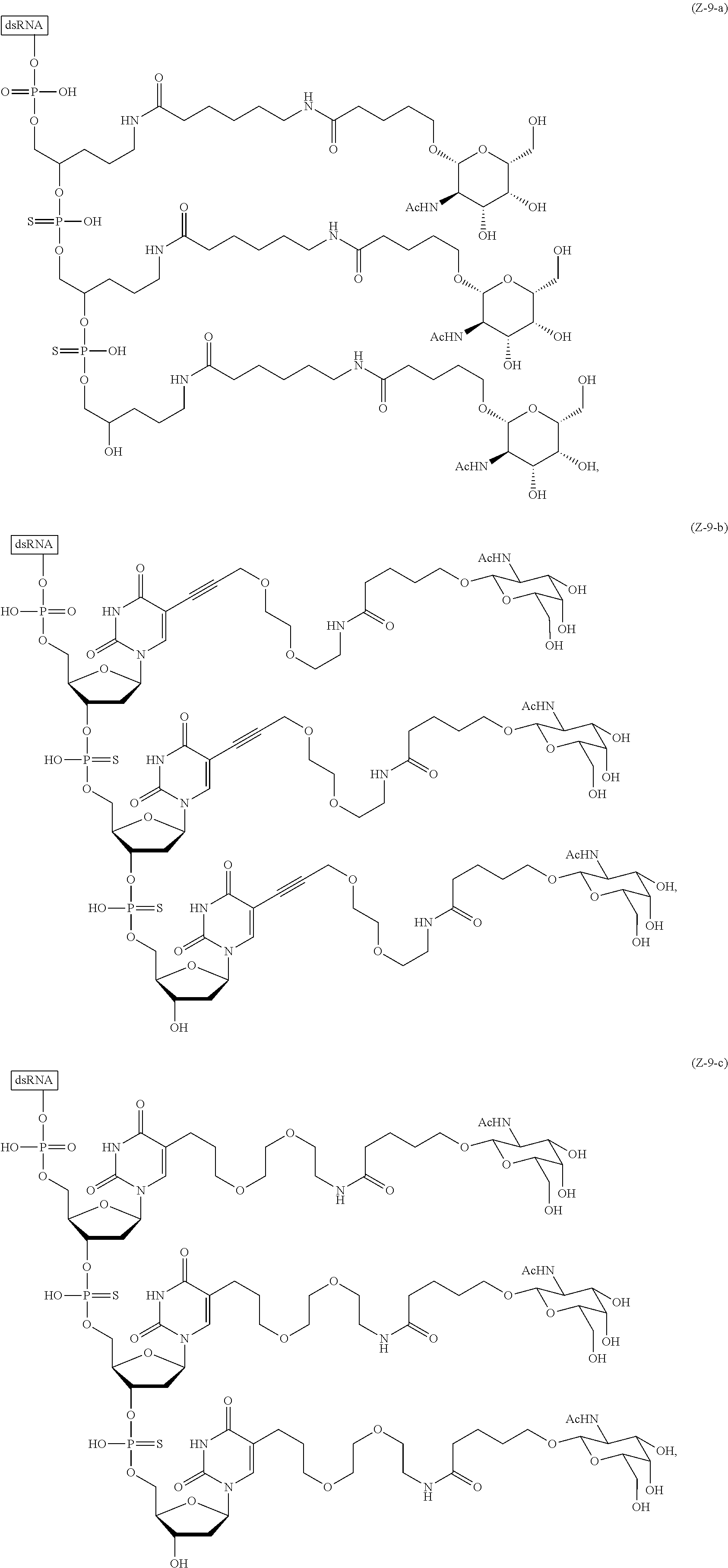

wherein the dsRNA includes any one of PCSK9 siRNA in Table 6i, the dsRNA includes.
   (i) a sense strand; and
   (ii) an antisense strand forming a duplex with the sense strand.

TABLE 6i

| | | | SEQ ID |
|---|---|---|---|
| siRNA | Sequence (5′-3′) | Strand | NO |
| 368 | A004p001U007p001C004pU007pU004pC007pA004pA007pG004p | SS | 2295 |
| | U007pU004pA007pC004pA007pA004pA007pA004pG004pC004pA | | |
| | 004pA004p001U004p001U004 | | |
| | U004p001U004p001G004pC004pU004pU004pU007pU004pG007p | AS | 2296 |
| | U007pA007pA004pC004pU004pU004pG004pA004pA004pG004pA | | |
| | 004pC004 | | |
| 369 | A004p001U007p001C004pU004pU004pC004pA004pA007pG004p | SS | 2297 |
| | U007pU004pA007pC004pA007pA004pA007pA004pG004pC004pA | | |
| | 004pA004p001U004p001U004 | | |
| | U004p001U004p001G004pC004pU004pU004pU007pU004pG007p | AS | 2298 |
| | U007pA007pA004pC004pU004pU004pG004pA004pA004pG004pA | | |
| | 004pC004 | | |
| 370 | A004p001U004p001C004pU004pU007pC004pA007pA004pG004p | SS | 2299 |
| | U007pU004pA007pC004pA007pA004pA007pA004pG004pC004pA | | |
| | 004pA004p001U004p001U004 | | |
| | U004p001U004p001G004pC004pU004pU004pU007pU004pG007p | AS | 2300 |
| | U007pA007pA004C004pU004pU004pG004pA004pA004pG004pA0 | | |
| | 04pA004 | | |
| 371 | A004p001U004p001C004pU004pU007pC004pA007pA004pG004p | SS | 2301 |
| | U007pU004pA007pC004pA007pA004pA007pA004pG004pC004pA | | |
| | 004pA004p001U004p001U004 | | |
| | U004p001U004p001G004pC004pU004pU004pU007pU004pG007p | AS | 2302 |
| | U007pA007pA004pC004pU004pU004pG004pA004pA004pG004pA | | |
| | 004pC004 | | |
| 372 | U004p001A007p001U004pC004pU004pU007pC004pA007pA004p | SS | 2303 |
| | G007pU004pU007pA004pC007pA004pA007pA004pA004pG004pC | | |
| | 004pA004p001U004p001U004 | | |
| | U004p001G004p001C004pU004pU004pU004pU007pG004pU007p | AS | 2304 |
| | A007pA007pC004pU004pU004pG004pA004pA004pG004pA004pU | | |
| | 004pA004 | | |
| 373 | U004p001A004p001U004pC004pU007pU004pC007pA004pA004p | SS | 2305 |
| | G007pU004pU007pA004pC007pA004pA007pA004pA004pG004pC | | |
| | 004pA004p001U004 | | |
| | U004p001G004p001C004pU004pU004pU004pU007pG004pU007p | AS | 2306 |
| | A007pA007pC004pU004pU004pG004pA004pA004pG004pA004pU | | |
| | 004pA004 | | |
| 374 | U004p001A007p001U004pC004pU004pU007pC004pA007pA004p | SS | 2307 |
| | G007pU004pU007pA004pC007pA004pA007pA004pA004pG004pC | | |
| | 004pA004p001U004p001U004 | | |
| | U004p001G004p001C004pU004pU004pU004pU007pG004pU007p | AS | 2308 |
| | A007pA002C004pU004pU004pG004pA004pA004pG004pA004pU0 | | |
| | 04pA004 | | |
| 375 | U004p001A007p001U004pC004pU004pU007pC004pA007pA004p | SS | 2309 |
| | G007pU004pU007pA004pC007pA004pA007pA004pA004pG004pC | | |
| | 004pA004p001U004p001U004 | | |
| | U004p001G004p001C004pU004pU004pU004pU007pG004pU007p | AS | 2310 |
| | A002pA007pC004pU004pU004pG004pA004pA004pG004pA004pU | | |
| | 004pA004 | | |
| 376 | U004p001A007p001U004pC004pU004pU007pC004pA007pA004p | SS | 2311 |
| | G007pU004pU007pA004pC007pA004pA007pA004pA004pG004pC | | |
| | 004pA004p001U004p001U004 | | |
| | U004p001G004p001C004pU004pU004pU004pU007pG004pT002p | AS | 2312 |
| | A007pA007pC004pU004pU004pG004pA004pA004pG004pA004pU | | |
| | 004pA004 | | |
| 377 | U004p001A007p001U004pC004pU004pU007pC004pA007pA004p | SS | 2313 |
| | G007pU004pU007pA004pC007pA004pA007pA004pA004pG004pC | | |
| | 004pA004p001U004p001U004 | | |
| | U004p001G004p001C004pU004pU004pU004pU007pG004pU007p | AS | 2314 |
| | A007pU007pC004pU004pU004pG004pA004pA004pG004pA004pU | | |
| | 004pA004 | | |
| 378 | U004p001A007p001U004pC004pU004pU007pC004pA007pA004p | SS | 2315 |
| | G007pU004pU007pA004pC007pA004pA007pA004pA004pG004pC | | |
| | 004pA004p001U004p001U004 | | |
| | U004p001G004p001C004pU004pU004pU004pU007pG004pU007p | AS | 2316 |
| | A007pC007pC004pU004pU004pG004pA004pA004pG004pA004pU | | |
| | 004pA004 | | |
| 379 | U004p001A007p001U004pC004pU004pU007pC004pA007pA004p | SS | 2317 |
| | G007pU004pU007pA004pC007pA004pA007pA004pA004pG004pC | | |
| | 004pA004p001U004p001U004 | | |
| | U004p001G004p001C004pU004pU004pU004pU007pG004pU007p | AS | 2318 |
| | A007pG007pC004pU004pU004pG004pA004pA004pG004pA004pU | | |
| | 004pA004 | | |
| 380 | A004p001U007p001C007pU004pU004pC007pA004pA007pG004p | SS | 2319 |
| | U007pU004pA007pC004pA007pA004pA007pA004pG004pC004pA | | |
| | 004pA004p001U004p001U004 | | |
| | U004p001U004p001G004pC004pU004pU004pU007pU004pG007p | AS | 2320 |
| | U007pA007pA004pC004pU004pU004pG004pA004pA004pG004pA | | |
| | 004pU004 | | |
| 381 | A004p001U007p001C007pU004pU004pC007pA004pA007pG004p | SS | 2321 |
| | U007pU004pA007pC004pA007pA004pA007pA004pG004pC004pA | | |
| | 004pA004p001U004p001U004 | | |
| | U004p001U004p001G004pC004pU004pU004pU007pU004pG007p | AS | 2322 |
| | U007pA007pA004pC004pU004pU004pG004pA004pA004pG004pA | | |
| | 004pC004 | | |
| 382 | A004p001U007p001C004pU004pU004pC004pA004pA007pG004p | SS | 2323 |
| | U004pU004pA007pC004pA007pA004pA007pA004pG004pC004pA | | |
| | 004pA004p001U004p001U004 | | |
| | U004p001U004p001G004pC004pU004pU004pU007pU004pG007p | AS | 2324 |
| | U007pA007pA004pC004pU004pU004pG004pA004pA004pG004pA | | |
| | 004pC004 | | |
| 383 | A004p001U007p001C004pU004pU004pC004pA004pA007pG004p | SS | 2325 |
| | U007pU004pA004pC004pA007pA004pA007pA004pG004pC004pA | | |
| | 004pA004p001U004p001U004 | | |
| | U004p001U004p001G004pC004pU004pU004pU007pU004pG007p | AS | 2326 |
| | U007pA007pA004pC004pU004pU004pG004pA004pA004pG004pA | | |
| | 004pU004 | | |
| 384 | A004p001U007p001C004pU004pU004pC004pA004pA007pG004p | SS | 2327 |
| | U007pU004pA004pC004pA007pA004pA007pA004pG004pC004pA | | |
| | 004pA004p001U004p001U004 | | |
| | U004p001U004p001G004pC004pU004pU004pU007pU004pG007p | AS | 2328 |
| | U007pA007pA004pC004pU004pU004pG004pA004pA004pG004pA | | |
| | 004pC004 | | |
| 385 | A004p001U007p001C004pU004pU004pC007pA004pA007pG004p | SS | 2329 |
| | U007pU004pA007pC004pA007pA004pA007pA004pG004pC004pA | | |
| | 004pA004p001U004p001U004 | | |
| | U004p001U004p001G004pC004pU004pU004pU007pU004pG007p | AS | 2330 |
| | U007pA007pA004pC004pU004pU004pG004pA004pA004pG004pA | | |
| | 004pC004 | | |

Specific codes in the nucleotide sequences are indicated in above, e.g., Tables A and A-1.

Additional suitable second dsRNAi agent targeting PCSK9 in Table 6i, or variants thereof and synthesis thereof are also described in WO2023/017004, entire contents of which are incorporated herein by reference.

In some embodiment, the second dsRNAi agent may have a structure of

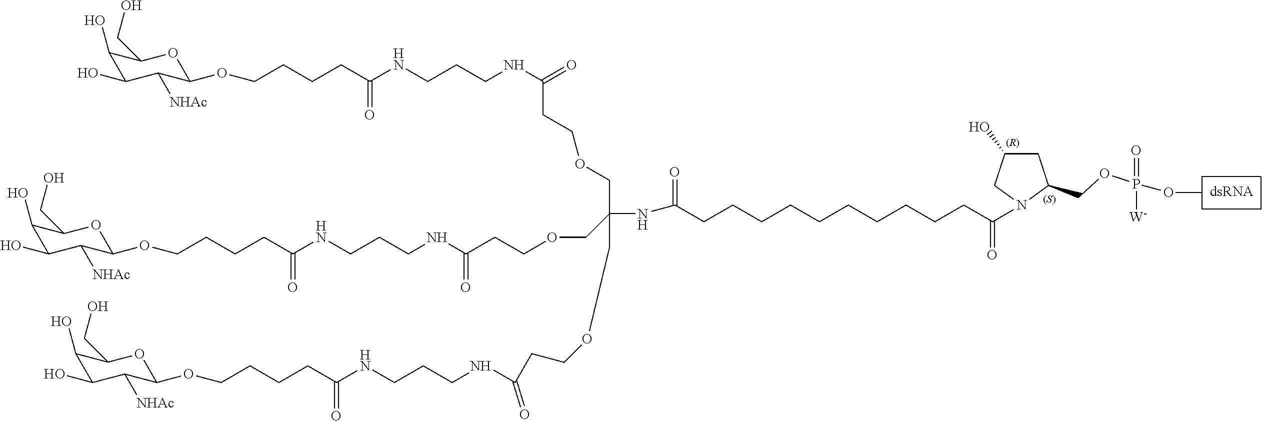

wherein W is S or O and the dsRNA includes any one of PCSK9 siRNA in Table 6j, the dsRNA includes:
   (i) a sense strand; and
   (ii) an antisense strand forming a duplex with the sense strand.

TABLE 6j

| PCSK9 | | | SEQ ID |
|---|---|---|---|
| siRNA | Sequence (5′-3′) | Strand | NO |
| 386 | G000pU000pU000pU000pU000pG000pC000pU000pU000pU000pU00 | SS | 2331 |
| | 0pG000pU000pA000pA000pC000pU000pU000pG000pA000pA000 | | |
| | U000pU000pC000pA000pA000pG000pU000pU000pA000pC000pA00 | AS | 2332 |
| | 0pA000pA000pA000pG000pC000pA000pA000pA000pA000pC000pA | | |
| | 000pG000 | | |
| 387 | G004p001U004p001U004pU004pU004pG004pC007pU004pU007pU0 | SS | 2333 |
| | 07pU007pG004pU004pA004pA004pC004pU004pU004pG004pA004p | | |
| | A004 | | |
| | U004p001U007p001C004pA004pA004pG007pU004pU004pA004pC0 | AS | 2334 |
| | 04pA004pA004pA004pA007pG004pC007pA004pA004pA004pA004p | | |
| | C004p001A004p001G004 | | |
| 388 | G007p001U004p001U007pU004pU007pG004pC007pU004pU007pU0 | SS | 2335 |
| | 07pU007pG004pU007pA004pA007pC004pU007pU004pG007pA004p | | |
| | A007 | | |
| | U004p001U007p001C004pA007pA004pG007pU004pU007pA004pC0 | AS | 2336 |
| | 07pA004pA004pA004pA007pG004pC007pA004pA007pA004pA007p | | |
| | C004p001A004p001G004 | | |
| 389 | G004p001U004p001U004pU004pU004pG004pC007pU007pU007pU0 | SS | 2337 |
| | 04pU004pG004pU004pA004pA004pC004pU004pU004pG004pA004p | | |
| | A004 | | |
| | U004p001U007p001C004pA004pA004pG004pU004pU007pA004pC0 | AS | 2338 |
| | 04pA004pA004pA004pA004pG004pC007pA004pA007pA004pA004p | | |
| | C004p001A004p001G004 | | |
| 390 | G004p001U004p001U004pU004pU004pG004pC007pU004pU007pU0 | SS | 2339 |
| | 07pU007pG004pU004pA004pA004pC004pU004pU004pG004pA004p | | |
| | A004 | | |
| | U004p001U007p001C004pA004pA004pG007pU004pU007pA004pC0 | AS | 2340 |
| | 04pA004pA004pA004pA007pG004pC007pA004pA004pA004pA004p | | |
| | C004p001A004p001G004 | | |
| 391 | G004p001U004p001U004pU004pU004pG004pC007pU004pU007pU0 | SS | 2341 |
| | 07pU007pG004pU004pA004pA004pC004pU004pU004pG004pA004p | | |
| | A004 | | |
| | U004p001U007p001C004pA004pA004pG007pU004pU004pA007pC0 | AS | 2342 |
| | 04pA004pA004pA004pA007pG004pC007pA004pA004pA004pA004p | | |
| | C004p001A004p001G004 | | |
| 392 | G004p001U004p001U004pU004pU004pG004pC004pU004pU007pU0 | SS | 2343 |
| | 07pU007pG004pU004pA004pA004pC004pU004pU004pG004pA004p | | |
| | A004 | | |
| | U004p001U007p001C004pA004pA004pG007pU004pU007pA004pC0 | AS | 2344 |
| | 04pA004pA004pA004pA007pG004pC007pA004pA004pA004pA004p | | |
| | C004p001A004p001G004 | | |
| 393 | G004p001U004p001U004pU004pU004pG004pC004pU004pU007pU0 | SS | 2345 |
| | 07pU007pG004pU004pA004pA004pC004pU004pU004pG004pA004p | | |
| | A004 | | |
| | U004p001U007p001C004pA004pA004pG007pU004pU004pA007pC0 | AS | 2346 |
| | 04pA004pA004pA004pA007pG004pC007pA004pA004pA004pA004p | | |
| | C004p001A004p001G004 | | |
| 394 | G004p001U004p001U004pU004pU004pG004pC004pU004pU007pU0 | SS | 2347 |
| | 07pU007pG004pU004pA004pA004pC004pU004pU004pG004pA004p | | |
| | A004 | | |
| | U004p001U007p001C004pA004pA004pG007pU004pU004pA004pC0 | AS | 2348 |
| | 04pA004pA004pA004pA007pG004pC007pA004pA004pA004pA004p | | |
| | C004p001A004p001G004 | | |
| 395 | G000pU000pU000pU000pU000pG000pC000pU000pU000pU000pU00 | SS | 2349 |
| | 0pG000pU000pA000pA000pC000pU000pU000pG000pA000pA000 | | |
| | U000pU000pC000pA000pA000pG000pU000pU000pA000pC000pA00 | AS | 2350 |
| | 0pA000pA000pA000pG000pC000pA000pA000pA000pA000pC000pU | | |
| | 000pU000 | | |
| 396 | G004p001U004p001U004pU004pU004pG004pC007pU004pU007pU0 | SS | 2351 |
| | 04pU004pG004pU004pA004pA004pC004pU004pU004pG004pA004p | | |
| | A004 | | |
| | U004p001U007p001C004pA007pA007pG007pU004pU007pA004pC0 | AS | 2352 |
| | 07pA004pA007pA004pA007pG004pC007pA004pA007pA004pA004p | | |
| | C004p001U004p001U004 | | |
| 397 | G004p001U004p001U004pU004pU004pG004pC007pU004pU007pU0 | SS | 2353 |
| | 07pU007pG004pU004pA004pA004pC004pU004pU004pG004pA004p | | |
| | A004 | | |
| | U004p001U007p001C004pA004pA004pG007pU004pU007pA007pC0 | AS | 2354 |
| | 04pA004pA004pA004pA007pG004pC007pA004pA004pA004pA004p | | |
| | C004p001U004p001U004 | | |
| 398 | G004p001U004p001U004pU004pU004pG004pC007pU004pU007pU0 | SS | 2355 |
| | 07pU007pG004pU004pA004pA004pC004pU004pU004pG004pA004p | | |
| | A004 | | |
| | U004p001U007p001C004pA004pA004pG007pU004pU004pA004pC0 | AS | 2356 |
| | 04pA004pA004pA004pA007pG004pC007pA004pA004pA004pA004p | | |
| | C004p001U004p001U004 | | |
| 399 | G007p001U004p001U007pU004pU007pG004pC007pU004pU007pU0 | SS | 2357 |
| | 07pU007pG004pU007pA004pA007pC004pU007pU004pG007pA004p | | |
| | A007 | | |
| | U004p001U007p001C004pA007pA004pG007pU004pU007pA004pC0 | AS | 2358 |
| | 07pA004pA004pA004pA007pG004pC007pA004pA007pA004pA007p | | |
| | C004p001U004p001U004 | | |
| 400 | G004p001U004p001U004pU004pU004pG004pC007pU007pU007pU0 | SS | 2359 |
| | 04pU004pG004pU004pA004pA004pC004pU004pU004pG004pA004p | | |
| | A004 | | |
| | U004p001U007p001C004pA004pA004pG004pU004pU007pA004pC0 | AS | 2360 |
| | 04pA004pA004pA004pA004pG004pC007pA004pA007pA004pA004p | | |
| | C004p001U004p001U004 | | |
| 401 | U000pU000pU000pG000pC000pU000pU000pU000pU000pG000pU00 | SS | 2361 |
| | OpA000pA000pC000pU000pU000pG000pA000pA000 | | |
| | U000pU000pC000pA000pA000pG000pU000pU000pA000pC000pA00 | AS | 2362 |
| | 0pA000pA000pA000pG000pC000pA000pA000pA000pU000pU000 | | |
| 402 | U004p001U004p001U004pG004pC004pU004pU007pU004pU007pG0 | SS | 2363 |
| | 04pU004pA004pA004pC004pU004pU004pG004pA004pA004 | | |
| | U004p001U007p001C004pA007pA007pG007pU004pU007pA004pC0 | AS | 2364 |
| | 07pA004pA007pA004pA007pG004pC007pA004pA007pA004p001U0 | | |
| | 04p001U004 | | |
| 403 | U004p001U004p001U004pG004pC004pU004pU007pU004pU007pG0 | SS | 2365 |
| | 07pU007pA004pA004pC004pU004pU004pG004pA004pA004 | | |
| | U004p001U007p001C004pA004pA004pG007pU004pU007pA004pC0 | AS | 2366 |
| | 04pA004pA007pA004pA007pG004pC007pA004pA007pA004p001U0 | | |
| | 04p001U004 | | |

Specific codes in the nucleotide sequences are indicated in above, e.g., Tables A and A-1.

Additional suitable second dsRNAi agent targeting PCSK9 in Table 6j, or variants thereof and synthesis thereof are also described in WO2023/051822, entire contents of which are incorporated herein by reference.

In some embodiment, the second dsRNAi agent includes a dsRNA and a cationic polymer (e.g., polyetherimide) and/or a cationic lipid, wherein the dsRNA includes any one of PCSK9 siRNA in Table 6k and the dsRNA includes:
(i) a sense strand; and
(ii) an antisense strand forming a duplex with the sense strand.

TABLE 6k

| PCSK9 | | | SEQ ID |
|---|---|---|---|
| siRNA | Sequence (5′-3′) | Strand | NO |
| 404 | C000pC000p0000pC000pA000pU000pA000pG000pG000pC000pC | SS | 2367 |
| | 000pU000pG000pG000pA000pG000pU000p0000 | | |
| | A000pA000pC000pU000pC000pC000pA000pG000pG000pC000C | AS | 2368 |
| | 000pU000pA000pU000pG000pA000pG000pG000 | | |
| 405 | C000p001C000p001U000pC000pA000pU000pA000pG000pG000p | SS | 2369 |
| | C000pC000pT002pG004pG004pA004pG004pU004pU004pU004pA | | |
| | 004p001T002p001T002 | | |
| | A004p001A004p001C004pU004pC004pC004pA000pG000pG000p | AS | 2370 |
| | C000pC000pU000pA000pU000pG000pA000pG000pG000pG000pU | | |
| | 000p001T002p001T002 | | |
| 406 | C004pC004pU004pC004pA004pU004pA004pG004pG004pC004pC | SS | 2371 |
| | 004pT002pG004pG004pA004pG004pU004pU004pU004pA004p00 | | |
| | 1T002p001T002 | | |
| | A004pA004pC004pU004pC004pC004pA004pG004pG004pC004pC | AS | 2372 |
| | 004pU004pA004pU004pG004pA004pG004pG004pG004pU004pT0 | | |
| | 02p001T002 | | |

Specific codes in the nucleotide sequences are indicated in above, e.g., Tables A and A-1.

In some embodiments, the dsRNA in Table 6k, or variants thereof may be further conjugated or attached to a ligand (e.g., a ligand selected from compounds of formula (A) to (F) or subordinates thereof).

Additional suitable second dsRNAi agent targeting PCSK9 in Table 6k, or variants thereof and synthesis thereof are also described in CN116162620, entire contents of which are incorporated herein by reference.

In some embodiment, the second dsRNAi agent includes a dsRNA and a cationic polymer (e.g., polyetherimide) and/or a cationic lipid, wherein the dsRNA includes any one of PCSK9 siRNA in Table 61 and the dsRNA includes:
(i) a sense strand; and
(ii) an antisense strand forming a duplex with the sense strand.

TABLE 61

| PCSK9 | | | SEQ ID |
|---|---|---|---|
| siRNA | Sequence (5′-3′) | Strand | NO |
| 407 | G004pC004pC004pU004pG004pG004pA007pG007pU007pU004pU | SS | 2373 |
| | 004pA004pU004pU004pC004pG004pG004pA004pA004 | | |
| | U004pU007pC004pC004pG004pA004pA004pU004pA004pA004pA | AS | 2374 |
| | 004pC004pU004pC007pC004pA004pG004pG004pC004 | | |
| 408 | C004pC004pC004pU004pC004pA004pU007pA007pG007pG004pC | SS | 2375 |
| | 004pC004pU004pG004pG004pA004pG004pU004pU004 | | |
| | A004pA007pC004pU004pC004pC004pA004pG004pG004pC004pC | AS | 2376 |
| | 004pU004pA004pU007pG004pA004pG004pG004pG004pU004pG0 | | |
| | 04 | | |
| 409 | G004pC004pA004pC004pC004pC004pU007pC007pA007pU004pA | SS | 2377 |
| | 004pG004pG004pC004pC004pU004pG004pG004pA004 | | |
| | U004pC007pC004pA004pG004pG004pC004pC004pU004pA004pU | AS | 2378 |
| | 004pG004pA004pG007pG004pG004pU004pG004pC004pC004pG0 | | |
| | 04 | | |
| 410 | C004pA004pC004pC004pC004pU004pC004pA004pU007pA007pG | SS | 2379 |
| | 007pG004pC004pC004pU004pG004pG004pA004pG004pU004pU0 | | |
| | 04 | | |
| | A004pA007pC004pU004pC004pC004pA004pG004pG004pC004pC | AS | 2380 |
| | 004pU004pA004pU007pG004pA004pG004pG004pG004pU004pG0 | | |
| | 04pC004pC004 | | |
| 411 | C004pG004pG004pC004pA004pC004pC004pC004pU007pC007pA | SS | 2381 |
| | 007pU004pA004pG004pG004pC004pC004pU004pG004pG004pA0 | | |
| | 04 | | |
| | U004pC007pC004pA004pG004pG004pC004pC004pU004pA004pU | AS | 2382 |
| | 004pG004pA004pG007pG004pG004pU004pG004pC004pC004pG0 | | |
| | 04pC004pU004 | | |
| 412 | C004pC004pC004pU004pC007pA004pU007pA007pG007pG004pC | SS | 2383 |
| | 004pC004pU004pG004pG004pA004pG004pU004pU004 | | |
| | A004pA007pC004pU004pC004pC007pA004pG007pG007pC004pC | AS | 2384 |
| | 004pU004pA004pU007pG004pA007pG004pG004pG004pU004pG0 | | |
| | 04 | | |
| 413 | G004pC004pA004pC004pC007pC004pU007pC007pA007pU004pA | SS | 2385 |
| | 004pG004pG004pC004pC004pU004pG004pG004pA004 | | |
| | U004pC007pC004pA004pG004pG007pC004pC007pU007pA004pU | AS | 2386 |
| | 004pG004pA004pG007pG004pG007pU004pG004pC004pC004pG0 | | |
| | 04 | | |
| 414 | C004pA004pC004pC004pC004pU004pC007pA004pU007pA007pG | SS | 2387 |
| | 007pG004pC004pC004pU004pG004pG004pA004pG004pU004pU0 | | |
| | 04 | | |
| | A004pA007pC004pU004pC004pC007pA004pG004pG004pC004pC | AS | 2388 |
| | 004pU004pA004pU007pG004pA007pG004pG004pG004pU004pG0 | | |
| | 04pC004pC004 | | |
| 415 | C004pG004pG004pC004pA004pC004pC007pC004pU007pC007pA | SS | 2389 |
| | 007pU004pA004pG004pG004pC004pC004pU004pG004pG004pA0 | | |
| | 04 | | |
| | U004pC007pC004pA004pG004pG007pC004pC004pU004pA004pU | AS | 2390 |
| | 004pG004pA004pG007pG004pG007pU004pG004pC004pC004pG0 | | |
| | 04pC004pU004 | | |
| 415 | A004pC004pC004pC004pU004pC004pA007pU007pA007pG004pG | SS | 2391 |
| | 004pC004pC004pU004pG004pG004pA004pG004pU004 | | |
| | A004pC007pU004pC004pC004pA004pG004pG004pC004pC004pU | AS | 2392 |
| | 004pA004pU004pG007pA004pG004pG004pG004pU004pG004pC0 | | |
| | 04 | | |
| 416 | G004pC004pA004pC004pC004pC004pU004pC004pA007pU007pA | SS | 2393 |
| | 007pG004pG004pC004pC004pU004pG004pG004pA004pG004pU0 | | |
| | 04 | | |
| | A004pC007pU004pC004pC004pA004pG004pG004pC004pC004pU | AS | 2394 |
| | 004pA004pU004pG007pA004pG004pG004pG004pU004pG004pC0 | | |
| | 04pC004pG004 | | |
| 417 | A004pC004pC004pC004pU007pC004pA007pU007pA007pG004pG | SS | 2395 |
| | 004pC004pC004pU004pG004pG004pA004pG004pU004 | | |
| | A004pC007pU004pC004pC004pA007pG004pG007pC007pC004pU | AS | 2396 |
| | 004pA004pU004pG007pA004pG007pG004pG004pU004pG004pC0 | | |
| | 04 | | |
| 418 | G004pC004pA004pC004pC004pC004pU007pC004pA007pU007pA | SS | 2397 |
| | 007pG004pG004pC004pC004pU004pG004pG004pA004pG004pU0 | | |
| | 04 | | |
| | A004pC007pU004pC004pC004pA007pG004pG004pC004pC004pU | AS | 2398 |
| | 004pA004pU004pG007pA004pG007pG004pG004pU004pG004pC0 | | |
| | 04pC004pG004 | | |
| 419 | G004pC004pC004pU004pG004pG004pA007pG007pU007pU004pU | SS | 2399 |
| | 004pA004pU004pU004pC004pG004pG004pA004pA004 | | |
| | U004pU007pC004pC004pG004pA004pA004pU004pA004pA004pA | AS | 2400 |
| | 004pC004pU004pC007pC004pA004pG004pG004pC004pC004pU0 | | |
| | 04 | | |
| 420 | A004pG004pG004pC004pC004pU004pG004pG004pA007pG007pU | SS | 2401 |
| | 007pU004pU004pA004pU004pU004pC004pG004pG004pA004pA0 | | |
| | 04 | | |
| | U004pU007pC004pC004pG004pA004pA004pU004pA004pA004pA | AS | 2402 |
| | 004pC004pU004pC007pC004pA004pG004pG004pC004pC004pU0 | | |
| | 04pA004pU004 | | |
| 421 | G004pC004pC004pU004pG004pG004pA007pG007pU007pU004pU | SS | 2403 |
| | 004pA004pU004pU004pC004pG004pG004pA004pA004pT002pT0 | | |
| | 02 | | |
| | U004pU007pC004pC004pG004pA004pA004pU004pA004pA004pA | AS | 2404 |
| | 004pC004pU004pC007pC004pA004pG004pG004pC004pT002pT0 | | |
| | 02 | | |

Specific codes in the nucleotide sequences are indicated in above, e.g., Tables A and A-1.

In some embodiments, the dsRNA in Table 61, or variants thereof may be further conjugated or attached to a ligand (e.g., a ligand selected from compounds of formula (A) to (F) or subordinates thereof).

Additional suitable second dsRNAi agent targeting PCSK9 in Table 61, or variants thereof and synthesis thereof are also described in CN116162620, entire contents of which are incorporated herein by reference.

In some embodiment, the second dsRNAi agent includes any one of PCSK9 siRNA in Table 6m and the dsRNA includes:
(i) a sense strand; and
(ii) an antisense strand forming a duplex with the sense strand.

TABLE 6m

| PCSK9 | | | SEQ ID |
|---|---|---|---|
| siRNA | Sequence (5′-3′) | Strand | NO |
| 422 | C004p001A004p001A004pG004pC004pA004pA007pG007pC007p | SS | 2405 |
| | A004pG004pA004pC004pA004pU004pU004pU004pA004pU004 | | |
| | A004p001U007p001A004pA004pA004pU007pG004pU004pC004p | AS | 2406 |
| | U004pG004pC004pU004pU007pG004pC007pU004pU004pG004p0 | | |
| | 01G004p001G004 | | |
| 423 | A004p001G004p001C004pA004pA004pG004pC007pA007pG007p | SS | 2407 |
| | A004pC004pA004pU004pU004pU004pA004pU004pC004pU004 | | |
| | A004p001G007p001A004pU004pA004pA007pA004pU004pG004p | AS | 2408 |
| | U004pC004pU004pG004pC007pU004pU007pG004pC004pU004p0 | | |
| | 01U004p001G004 | | |
| 424 | A004p001A004p001G004pC004pA004pG004pA007pC007pA007p | SS | 2409 |
| | U004pU004pU004pA004pU004pC004pU004pU004pU004pU004 | | |
| | A004p001A007p001A004pA004pG004pA007pU004pA004pA004p | AS | 2410 |
| | A004pU004pG004pU004pC007pU004pG007pC004pU004pU004p0 | | |
| | 01G004p001C004 | | |
| 425 | C004p001U004p001A004pG004pA004pC004pC007pU007pG007p | SS | 2411 |
| | U004pU004pU004pU004pG004pC004pU004pU004pU004pU004 | | |
| | A004p001A007p001A004pA004pG004pC007pA004pA004pA004p | AS | 2412 |
| | A004pC004pA004pG004pG007pU004pC007pU004pA004pG004p0 | | |
| | 01A004p001A004 | | |
| 426 | U004p001U004p001U004pU004pG004pU004pA007pA007pC007p | SS | 2413 |
| | U004pU004pG004pA004pA004pG004pA004pU004pA004pU004 | | |
| | A004p001U007p001A004pU004pC004pU007pU004pC004pA004p | AS | 2414 |
| | A004pG004pU004pU004pA007pC004pA007pA004pA004pA004p0 | | |
| | 01G004p001C004 | | |
| 427 | U004p001U004p001U004pG004pU004pA004pA007pC007pU007p | SS | 2415 |
| | U004pG004pA004pA004pG004pA004pU004pA004pU004pU004 | | |
| | A004p001A007p001U004pA004pU004pC007pU004pU004pC004p | AS | 2416 |
| | A004pA004pG004pU004pU007pA004pC007pA004pA004pA004p0 | | |
| | 01A004p001G004 | | |
| 428 | U004p001U004p001G004pU004pA004pA004pC007pU007pU007p | SS | 2417 |
| | G004pA004pA004pG004pA004pU004pA004pU004pU004pU004 | | |
| | A004p001A007p001A004pU004pA004pU007pC004pU004pU004p | AS | 2418 |
| | C004pA004pA004pG004pU007pU004pA007pC004pA004pA004p0 | | |
| | 01A004p001A004 | | |
| 429 | U004p001A004p001A004pC004pU004pU004pG007pA007pA007p | SS | 2419 |
| | G004pA004pU004pA004pU004pU004pU004pA004pU004pU004 | | |
| | A004p001A007p001U004pA004pA004pA007pU004pA004pU004p | AS | 2420 |
| | C004pU004pU004pC004pA007pA004pG007pU004pU004pA004p0 | | |
| | 01C004p001A004 | | |
| 430 | U004p001U004p001U004pG004pU004pA004pG007pC007pA007p | SS | 2421 |
| | U004pU004pU004pU004pU004pA004pU004pU004pA004pA004 | | |
| | U004p001U007p001A004pA004pU004pA007pA004pA004pA004p | AS | 2422 |
| | A004pU004pG004pC004pU007pA004pC007pA004pA004pA004p0 | | |
| | 01A004p001C004 | | |
| 431 | G004p001U004p001A004pG004pC004pA004pU007pU007pU007p | SS | 2423 |
| | U004pU004pA004pU004pU004pA004pA004pU004pA004pU004 | | |
| | A004p001U007p001A004pU004pU004pA007pA004pU004pA004p | AS | 2424 |
| | A004pA004pA004pA004pU007pG004pC007pU004pA004pC004p0 | | |
| | 01A004p001A004 | | |

Specific codes in the nucleotide sequences are indicated in above, e.g., Tables A and A-1.

In some embodiments, the dsRNA in Table 6m, or variants thereof may be conjugated or attached to a ligand (e.g., a ligand selected from compounds of formula (A) to (F) or subordinates thereof).

Additional suitable second dsRNAi agent targeting PCSK9 in Table 6m, or variants thereof and synthesis thereof are also described in CN117106781, entire contents of which are incorporated herein by reference.

In some embodiment, the second dsRNAi agent includes any one of PCSK9 siRNA in Table 61 and the dsRNA includes:
(i) a sense strand; and
(ii) an antisense strand forming a duplex with the sense strand.

TABLE 6n

| PCSK9 | | | SEQ ID |
|---|---|---|---|
| siRNA | Sequence (5′-3′) | Strand | NO |
| 432 | A004p001A004p001G004pA004pU004pC004pC007pU004pG007p | SS | 2425 |
| | C007pA007pU004pG004pU004pC004pU004pU007pC004pC004pA | | |
| | 004pU004 | | |
| | A004p001U007pG004pG004pA004pA007pG1016pA004pC004pA0 | AS | 2426 |
| | 04pU004pG004pC004pA007pG004pG007pA004pU004pC004pU00 | | |
| | 4pU004p001G004p001G004 | | |
| 433 | A004p001A004p001G004pA004pU004pC004pC007pU004pG007p | SS | 2427 |
| | C007pA007pU004pG004pU004pC004pU004pU007pC004pC004pA | | |
| | 004pU004 | | |
| | X033A1027p001U007p001G004pG004pA004pA007pG004pA004p | AS | 2428 |
| | C004pA004pU004pG004pC004pA007pG004pG007pA004pU004pC | | |
| | 004pU004pU004p001G004p001G004 | | |
| 434 | U004p001G004p001G004pA004pG004pG004pC007pU004pU007p | SS | 2429 |
| | A007pG007pC004pU004pU004pU004pC004pU007pG004pG004pA | | |
| | 004pU004 | | |
| | A004p001U007p001C007pC007pA004pG007pA004pA004pA004p | AS | 2430 |
| | G004pC004pU004pA004pA007pG004pC007pC004pU004pC004pC | | |
| | 004pA004p001U004p001U004 | | |
| 435 | U004p001G004p001G004pA004pG004pG004pC007pU004pU007p | SS | 2431 |
| | A007pG007pC004pU004pU004pU004pC004pU007pG004pG004pA | | |
| | 004pU004 | | |
| | A004p001U007p001C007pC007pA004pG007pA1016pA004pA004 | AS | 2432 |
| | pG004pC004pU004pA004pA007pG004pC007pC004pU004pC004p | | |
| | C004pA004p001U004p001U004 | | |
| 436 | U004p001G004p001G004pA004pG004pG004pC007pU004pU007p | SS | 2433 |
| | A007pG007pC004pU004pU004pU004pC004pU007pG004pG004pA | | |
| | 004pU004 | | |
| | X033A1027p001U007p001C007pC007pA004pG007pA004pA004p | AS | 2434 |
| | A004pG004pC004pU004pA004pA007pG004pC007pC004pU004pC | | |
| | 004pC004pA004p001U004p001U004 | | |
| 437 | C004p001U004p001U004pU004pU004pG004pU007pA004pA007p | SS | 2435 |
| | C007pU007pU004pG004pA004pA004pG004pA007pU004pA004pU | | |
| | 004pU004 | | |
| | A004p001A007p001U004pA004pU004pC007pU004pU004pC004p | AS | 2436 |
| | A004pA004pG004pU004pU007pA004pC007pA004pA004pA004pA | | |
| | 004pG004p001C004p001A004 | | |
| 438 | C004p001U004p001U004pU004pU004pG004pU007pA004pA007p | SS | 2437 |
| | C007pU007pU004pG004pA004pA004pG004pA007pU004pA004pU | | |
| | 004pU004 | | |
| | X033A1027p001A007p001U004pA007pU007pC007pU004pU004p | AS | 2438 |
| | C004pA004pA004pG004pU004pU007pA004pC007pA004pA004pA | | |
| | 004pA004pG004p001C004p001A004 | | |
| 439 | C004p001U004p001U004pU004pU004pG004pU007pA004pA007p | SS | 2439 |
| | C007pU007pU004pG004pA004pA004pG004pA007pU004pA004pU | | |
| | 004pU004 | | |
| | X033A1027p001A007p001U004pA007pU007pC007pU1016pU004 | AS | 2440 |
| | pC004pA004pA004pG004pU004pU007pA004pC007pA004pA004p | | |
| | A004pA004pG004p001C004p001A004 | | |
| 440 | C004p001U004p001U004pU004pU004pG004pU007pA004pA007p | SS | 2441 |
| | C004pU007pU004pG004pA004pA004pG004pA007pU004pA004pU | | |
| | 004pU004 | | |
| | A004p001A007p001U007pA007pU004pC007pU004pU004pC004p | AS | 2442 |
| | A004pA004pG004pU004pU007pA004pC007pA004pA004pA004pA | | |
| | 004pG004p001C004p001A004 | | |
| 441 | G004p001A004p001A004pG004pA004pU004pA007pU004pU007p | SS | 2443 |
| | U007pA007pU004pU004pC004pU004pG004pG007pG004pU004pU | | |
| | 004pU004 | | |
| | X033A1027p001A007p001A004pC007pC007pC007pA004pG004p | AS | 2444 |
| | A004pA004pU004pA004pA004pA007pU004pA007pU004pC004pU | | |
| | 004pU004pC004p001A004p001A004 | | |
| 442 | G004p001A004p001A004pG004pA004pU004pA007pU004pU007p | SS | 2445 |
| | U004pA007pU004pU004pC004pU004pG004pG007pG004pU004pU | | |
| | 004pU004 | | |
| | A004p001A007p001A004pC007pC007pC007pA004pG004pA004p | AS | 2446 |
| | A004pU004pA004pA004pA007pU004pA007pU004pC004pU004pU | | |
| | 004pC004p001A004p001A004 | | |
| 443 | G004p001A004p001A004pG004pA004pU004pA007pU004pU007p | SS | 2447 |
| | U004pA007pU004pU004pC004pU004pG004pG007pG004pU004pU | | |
| | 004pU004 | | |
| | X033A1027p001A007p001A004pC007pC007pC007pA004pG004p | AS | 2448 |
| | A004pA004pU004pA004pA004pA007pU004pA007pU004pC004pU | | |
| | 004pU004pC004p001A004p001A004 | | |
| 444 | G004p001U004p001U004pU004pU004pG004pU007pA004pG007p | SS | 2449 |
| | C007pA007pU004pU004pU004pU004pU004pA007pU004pU004pA | | |
| | 004pA004 | | |
| | U004p001U007p001A007pA007pU004pA007pA004pA004pA004p | AS | 2450 |
| | A004pU004pG004pC004pU007pA004pC007pA004pA004pA004pA | | |
| | 004pC004p001C004p001C004 | | |
| 445 | G004p001U004p001U004pU004pU004pG004pU007pA004pG007p | SS | 2451 |
| | C007pA007pU004pU004pU004pU004pU004pA007pU004pU004pA | | |
| | 004pA004 | | |
| | X033U1027p001U007p001A004pA007pU007pA007pA004pA004p | AS | 2452 |
| | A004pA004pU004pG004pC004pU007pA004pC007pA004pA004pA | | |
| | 004pA004pC004p001C004p001C004 | | |
| 446 | G004p001U004p001U004pU004pU004pG004pU007pA004pG007p | SS | 2453 |
| | C007pA007pU004pU004pU004pU004pU004pA007pU004pU004pA | | |
| | 004pA004 | | |
| | X033U1027p001U007p001A004pA007pU007pA007pA1016pA004 | AS | 2454 |
| | pA004pA004pU004pG004pC004pU007pA004pC007pA004pA004p | | |
| | A004pA004pC004p001C004p001C004 | | |

Specific codes in the nucleotide sequences are indicated in above, e.g., Tables A and A-1.

In some embodiments, the dsRNA in Table 6n, or variants thereof may be conjugated or attached to a ligand having a structure of

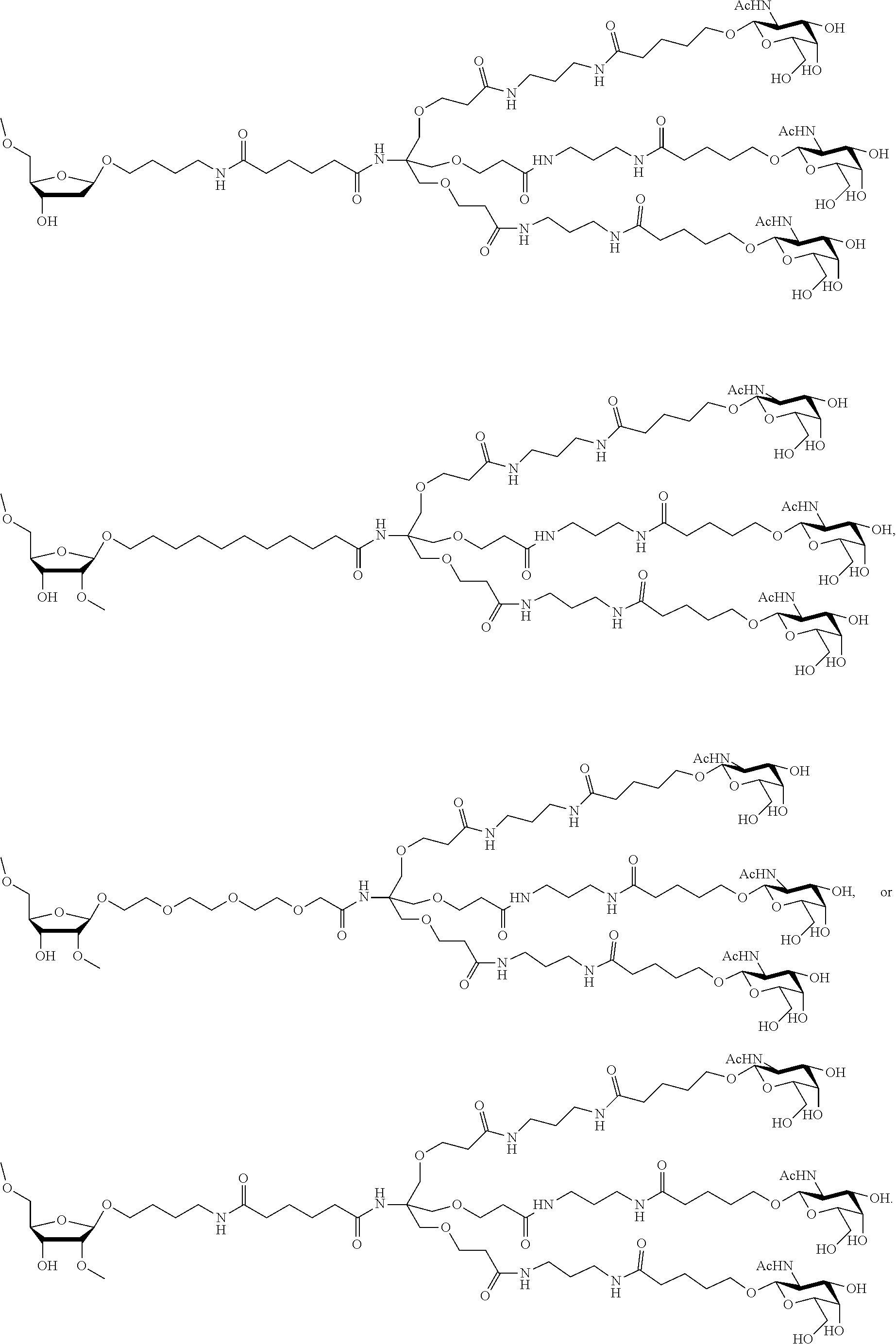

In some embodiments, the dsRNA in Table 6n, or variants thereof may be conjugated or attached to a ligand (e.g., a ligand selected from compounds of formula (A) to (F) or subordinates thereof).

Additional suitable second dsRNAi agent targeting PCSK9 in Table 6n, or variants thereof and synthesis thereof are also described in CN117210468, entire contents of which are incorporated herein by reference.

In some embodiment, the second dsRNAi agent may have a structure of

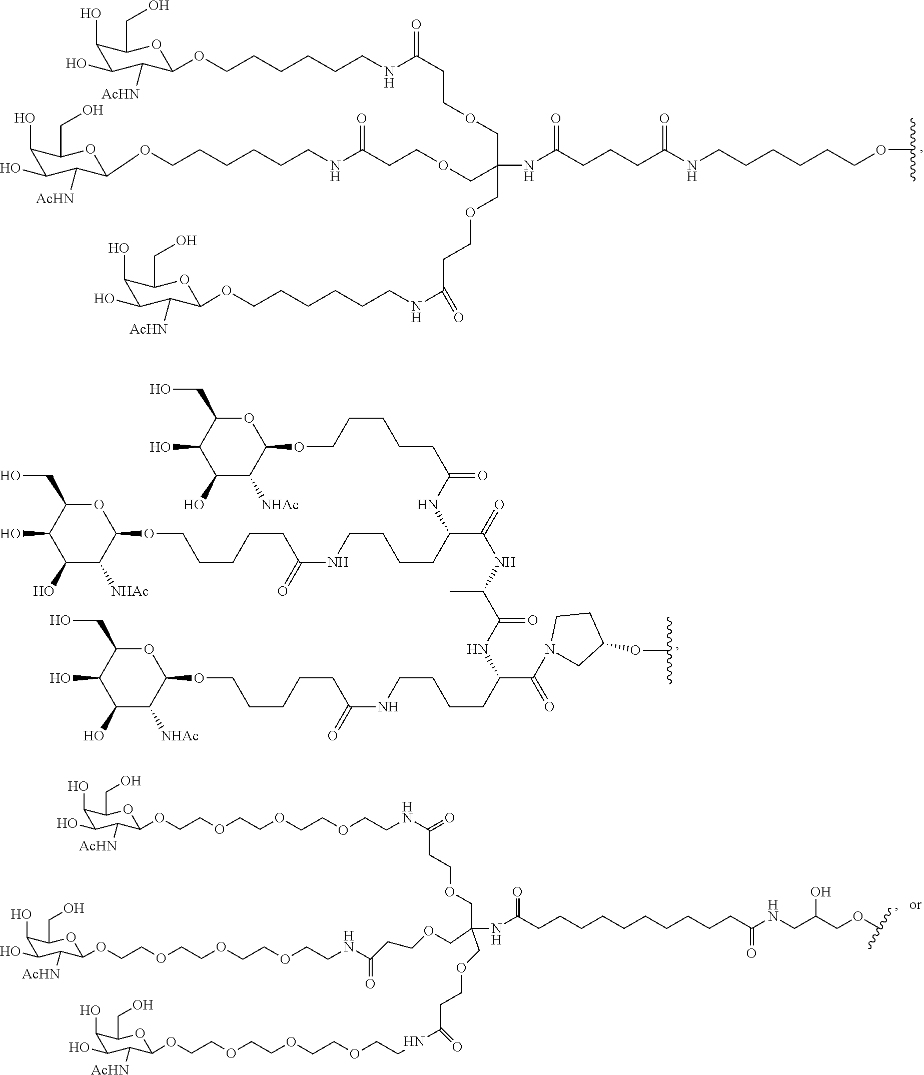

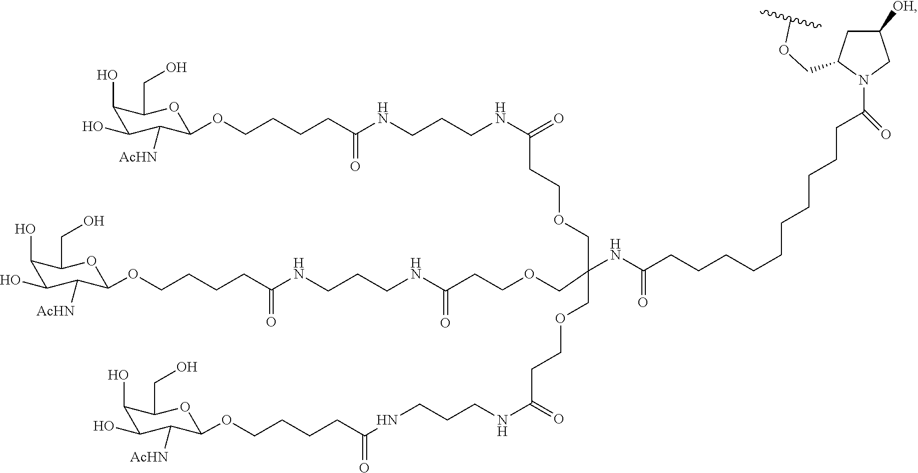 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

and
a dsRNA selected from siRNAs in Table 6o, which is attached or conjugated to the ligand, wherein the dsRNA includes any one of PCSK9 siRNA in Table 6o and the dsRNA includes:
(i) a sense strand; and
(ii) an antisense strand forming a duplex with the sense strand

TABLE 60

| PCSK9 | | | SEQ ID |
|---|---|---|---|
| siRNA | Sequence (5′-3′) | Strand | NO |
| 447 | A004pG004pA004pC004pC004pU004pG007pU004pU007pU007pU | SS | 2455 |
| | 007pG004pC004pU004pU004pU004pU004pG004p001U004p001B | | |
| | 001 | | |
| | A004p001C007p001A004pA004pA004pA007pG004pC004pA004p | AS | 2456 |
| | A004pA004pA004pC004pA007pG004pG007pU004pC004pU004p0 | | |
| | 01A004p001G004 | | |
| 448 | B001p001A004p001G004pA004pC004pC004pU004pG007pU004p | SS | 2457 |
| | U007pU007p0007pG004pC004p0004pU004pU004pU004pG004pU | | |
| | 004 | | |
| | A004p001C007p001A004pA004pA004pA007pG004pC004pA004p | AS | 2458 |
| | A004pA004pA004pC004pA007pG004pG007pU004pC004pU004p0 | | |
| | 01A004p001G004 | | |

Specific codes in the nucleotide sequences are indicated in above, e.g., Tables A and A-1.

In some embodiments, the dsRNA in Table 6o, or variants thereof may be conjugated or attached to a ligand (e.g., a ligand selected from compounds of formula (A) to (F) or subordinates thereof).

Additional suitable second dsRNAi agent targeting PCSK9 in Table 6o, or variants thereof and synthesis thereof are also described in CN117384907, entire contents of which are incorporated herein by reference.

In some embodiments, the additional therapeutic agent includes the apoprotein (e.g., ApoA1, ApoC3, or ApoE) inhibitor. In some embodiments, the ApoA1 inhibitor may be a small molecule, antibody, peptide, or a therapeutic oligonucleotides (e.g., antisense oligonucleotide or “ASO”). In some embodiments, the additional therapeutic agent includes an antisense oligonucleotide targeting ApoA1 or ApoC3. In some embodiments, the additional therapeutic agent includes pelacarsen. In some embodiments, the additional therapeutic agent includes olezarsen.

In some embodiments, the additional therapeutic agent includes lipoprotein (a) (LPA) inhibitor. In some embodiments, the additional therapeutic agent is an antisense oligonucleotide targeting LPA, e.g., pelacarsen. The antisense oligonucleotide includes a nucleotide sequence of Oligo Nos. D1-D3 in Table 6p-1.

TABLE 6p-1

| | | SEQ |
|---|---|---|
| | | ID |
| Oligo no. | Antisense strand | NO: |
| D1 | UGCUCCGTTGGTGCTUGUUC | 2459 |
| D2 | ^{Me}UsGo^{Me}Co^{Me}Uo^{Me}Co^{Me}CsGsTsTsGsGsTsGs^{Me}CsTs^{Me}UoGo^{Me}Us | 2460 |
| | ^{Me}Us^{Me}C | |
| D3 | THA-AHo- | 2461 |
| (pelacarsen) | ^{Me}UsGo^{Me}Co^{Me}Uo^{Me}Co^{Me}CsGsTsTsGsGsTsGs^{Me}CsTs^{Me}UoGo^{Me}Us | |
| | ^{Me}Us^{Me}C | |

In Table 6p-1, “s” represents phosphorothioate (PS) linkage, “o” represents phosphodiester linkage, each “A,” “G,” “C,” and “T”, represents each deoxyribonucleic acid, “^{Me}” represents methylated modification on a nucleobase (e.g., “^{Me}U”), each “A,” “G,” “C,” and “U”, represents each ribonucleic acid with 2′-MOE modification.

In the Oligo no. D3, 5′-trishexylamino (THA)-AHo (or “THA-C6-GalNAc3”) in Table 6p-1 has the structure of

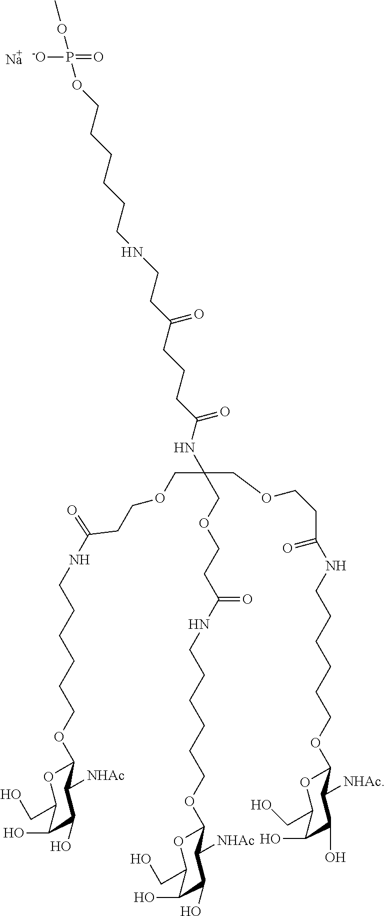

In the Oligo no. D3, THA in Table 6p-1 has the structure of

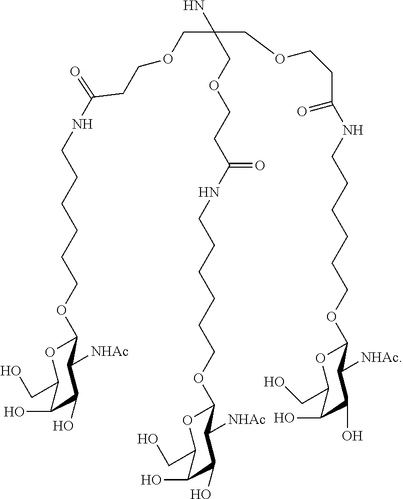

In some embodiments, the antisense oligonucleotide (pelacarsen) has the following structure (SEQ ID NO: 2461):

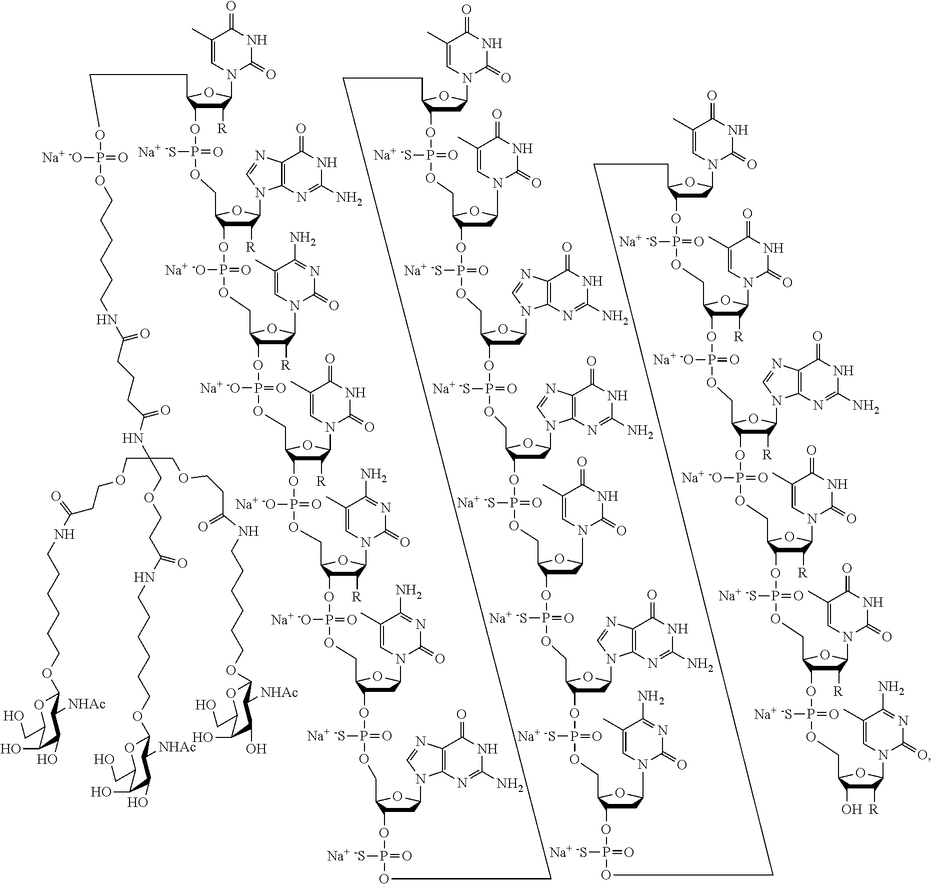

wherein R is —OCH₂CH₂OCH₃.

In some embodiments, the antisense oligonucleotide (pelacarsen) has the following structure (SEQ ID NO: 2461):

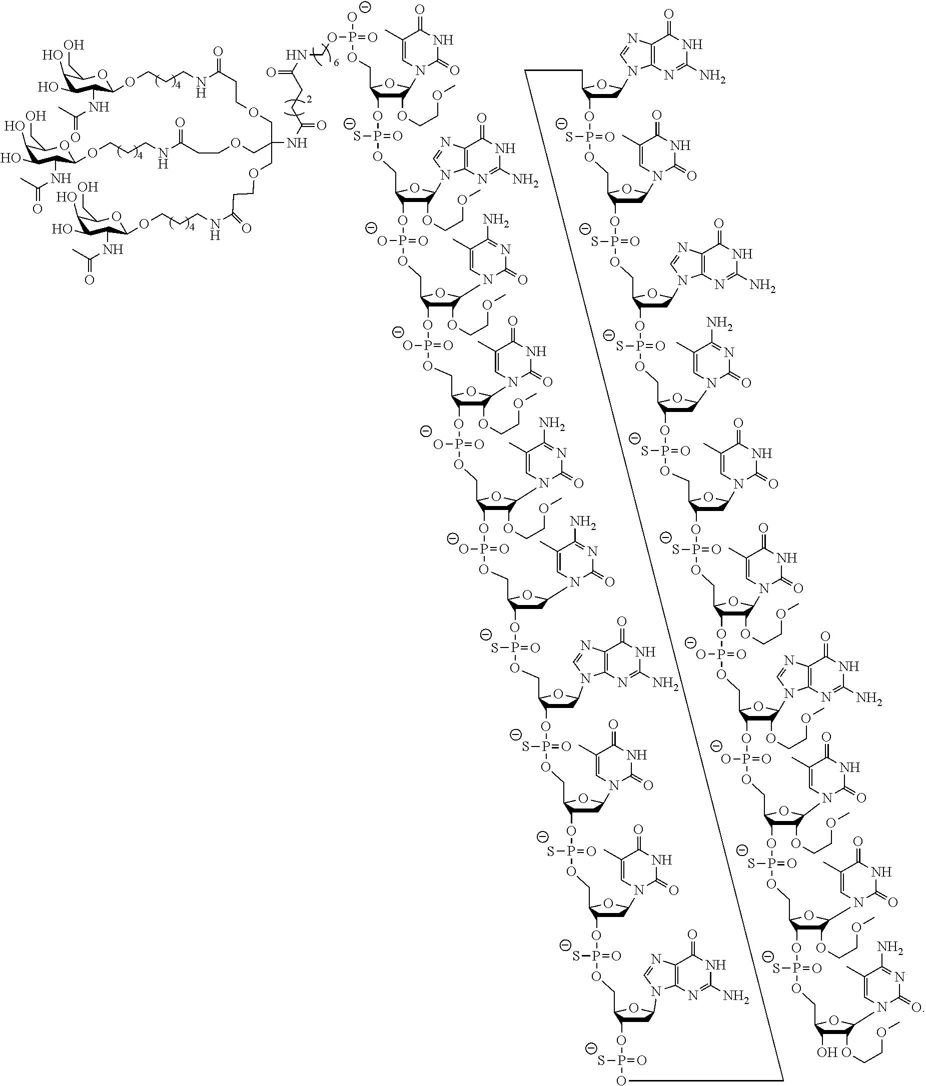

In some embodiments, the antisense oligonucleotide (pelacarsen) has the following structure (SEQ ID NO: 2461):

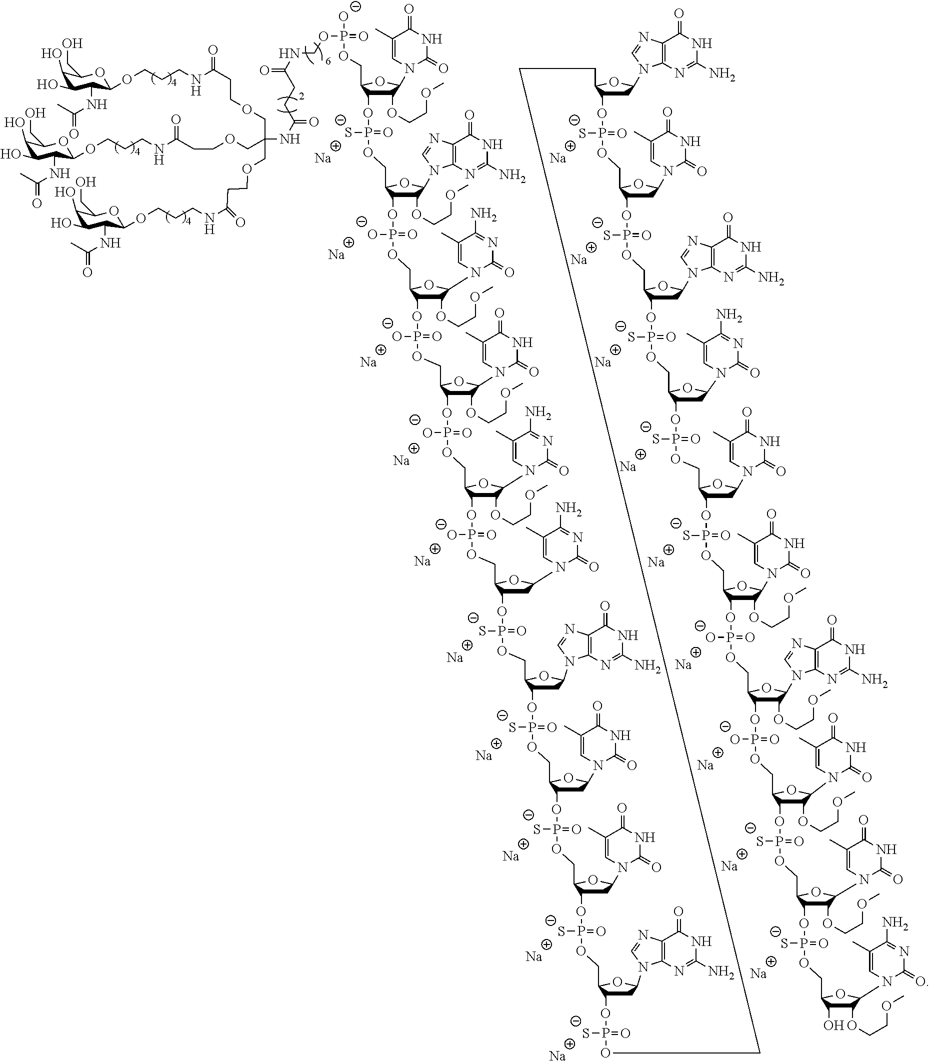

In some embodiments, the additional therapeutic agent includes lipoprotein (a) (LPA), or otherwise Apo(a) inhibitor. In some embodiments, the LPA (ApoA) inhibitor siRNA includes a dsRNA in Tables 6p-2 to 6p-7.

TABLE 6p-2

| LPA | | | SEQ ID |
|---|---|---|---|
| siRNA | Sequence (5′-3′) | Strand | NO |
| L1 | GAGAGUUAUCGAGGCACAUAA | SS | 2462 |
| | UUAUGUGCCUCGAUAACUCUC | AS | 2463 |
| L2 | (GLS- | SS | 2464 |
| | 15) p001 (Invab) p001G004pA004pG004pA004pG004pU004pU00 | | |
| | 4pA004pU007pC004pG007pA004pG007pG004pC004pA004pC004 | | |
| | pA004pU004pA004pA004p001 (Invab) | | |
| | U004p001U007p001A004pU004pG004pU004pG007pC004pC004p | AS | 2465 |
| | U004pC004pG007pA004pU007pA004pA007pC004pU004pC004p0 | | |
| | 01U004p001C004 | | |
| L3 | (GLS- | SS | 2466 |
| | 15) p001 (Imann) p001G004pA004pG004pA004pG004pU004pU00 | | |
| | 4pA004pU007pC004pG007pA004pG007pG004pC004pA004pC004 | | |
| | pA004pU004pA004p001A004p001 (Imann) | | |
| | U007p0010007p001A004pU004pG004pU004pG007pC004pC004p | AS | 2467 |
| | U004pC004pG007pA004pU007pA004pA007pC004pU004pC004p0 | | |
| | 01U004p001C004 | | |

GLS-15: 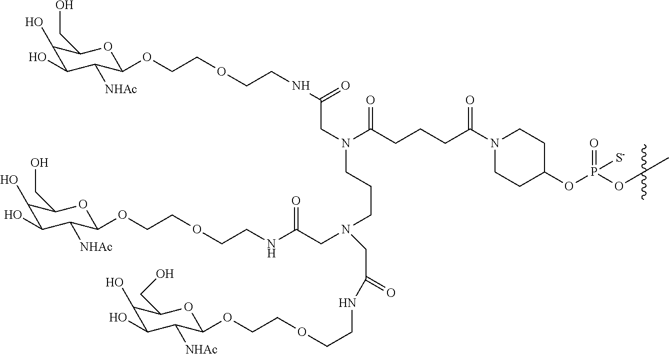 [SMILES: *OP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1]
Imann: 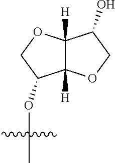 [SMILES: *O[C@@H]1CO[C@H]2[C@@H]1OC[C@H]2O]
or 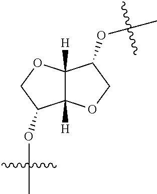 [SMILES: *O[C@@H]1CO[C@H]2[C@@H]1OC[C@H]2O*]
invab = inverted abasic

Specific codes in the nucleotide sequences are indicated in above, e.g., Tables A and A-1.

Additional suitable additional dsRNAi agent targeting LPA, or variants thereof and synthesis thereof are also described in WO2023/138689, entire contents of which are incorporated herein by reference.

TABLE 6p-3

| | | SEQ | | SEQ |
|---|---|---|---|---|
| siRNA | | ID | | ID |
| no. | Sense strand | NO: | Antisense strand | NO: |
| B1 | p001(Imann)p001G004pA004p | 2468 | U004p001U007p001A004pU004pG00 | 2469 |
| | G004pA004pG004pU004pU004p | | 4pU004pG007pC004pC004pU004pC0 | |
| | A004pU007pC004pG007pA004p | | 04pG007pA004pU007pA004pA007pC | |
| | G007pG004pC004pA004pC004p | | 004pU004pC004p001U004p001C004 | |
| | A004pU004pA004p001A004p00 | | | |
| | 1(Imann) | | | |

In Table 6p-3, (Imann),when at the end of each strand is

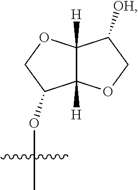 [SMILES: *O[C@@H]1CO[C@H]2[C@@H]1OC[C@H]2O]

or when further coupling to a delivery molecule, is

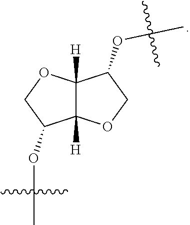 [SMILES: *O[C@@H]1CO[C@H]2[C@@H]1OC[C@H]2O*]

Other specific codes in the nucleotide sequences are indicated in above, e.g., Tables A and A-1.

TABLE 6p-4

| | | SEQ | | SEQ |
|---|---|---|---|---|
| siRNA | | ID | | ID |
| no. | Sense strand | NO: | Antisense strand | NO: |
| B2 | p001(Invab)p001G004pA004p | 2470 | U004p001U007p001A004pU004pG00 | 2473 |
| | G004pA004pG004pU004pU004p | | 4pU004pG007pC004pC004pU004pC0 | |
| | A004pU007pC004pG007pA004p | | 04pG007pA004pU007pA004pA007pC | |
| | G007pG004pC004pA004pC004p | | 004pU004pC004p001U004p001C004 | |
| | A004pU004pA004pA004p001 | | | |
| | (Invab) | | | |
| B3 | p001(Imann)p001G004pA004p | 2471 | U007p001U007p001A004pU004pG00 | 2474 |
| | G004pA004pG004pU004pU004p | | 4pU004pG007pC004pC004pU004pC0 | |
| | A004pU007pC004pG007pA004p | | 04pG007pA004pU007pA004pA007pC | |
| | G007pG004pC004pA004pC004p | | 004pU004pC004p001U004p001C004 | |
| | A004pU004pA004p001A004p00 | | | |
| | 1(Imann) | | | |
| B4 | p001(Invab)p001G004pA004p | 2472 | U004p001U007p001A004pU004pG00 | 2475 |
| | G004pA004pG004pU004pU004p | | 4pU004pG007pC004pC004pU004pC0 | |
| | A004pU007pC004pG007pA004p | | 04pG007pA004pU007pA004pA007pC | |
| | G007pG004pC004pA004pC004p | | 004pU004pC004p001U004p001C004 | |
| | A004pU004pA004pA004p001 | | | |
| | (Invab) | | | |

In Table 6p-4, Imann is or

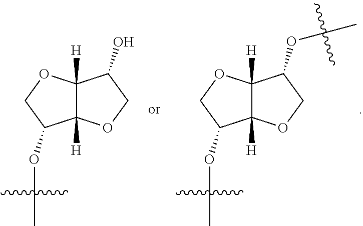 [SMILES: *O[C@@H]1CO[C@H]2[C@@H]1OC[C@H]2O ; *O[C@@H]1CO[C@H]2[C@@H]1OC[C@H]2O*]

and invab is inverted abasic modification:

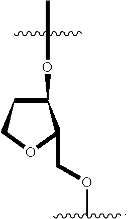

Other specific codes in the nucleotide sequences are indicated in above, e.g., Tables A and A-1.

TABLE 6p-5

| | | SEQ | | SEQ |
|---|---|---|---|---|
| SIRNA | | ID | | ID |
| no. | Sense strand | NO: | Antisense strand | NO: |
| B5 | C004p001A004pG004pC004pC0 | 2476 | U004p001C007p001G004pU007pA00 | 2478 |
| | 04pC004pC004pU004pU007pA0 | | 4pU007pA004pA004pC004pA004pA0 | |
| | 07pU007pU004pG004pU004pU0 | | 04pU007pA004pA007pG004pG007pG | |
| | 04pA004pU004pA004pC004pG0 | | 004pG007pC004p001U007p001G004 | |
| | 04p001 (invdA) | | | |
| B6 | G004pC004pC004pC004pC004p | 2477 | C007p001G004pU007pA004pU007pA | 2479 |
| | U004pU007pA007pU007pU004p | | 004pA004pC004pA004pA004pU007p | |
| | G004pU004pU004pA004pU004p | | A004pA007pG004pG007pG004pG007 | |
| | A004pC004pG004 | | pC004 | |

In Table 6p-5, (invdA) is an inverted deoxyriboadenosine (3′-3′ linked nucleotide). Other specific codes in the nucleotide sequences are indicated in above, e.g., Tables A and A-1.

TABLE 6p-6

| | | SEQ | | SEQ |
|---|---|---|---|---|
| SIRNA | | ID | | ID |
| no. | Sense strand | NO: | Antisense strand | NO: |
| B7 | C004p001G004pG004pU004pA004 | 2480 | A004p001U007p001A004pA007pC0 | 2483 |
| | pA004pU007pG007pG007pA004pC | | 04pU007pC004pU007pG004pU007p | |
| | 004pA004pG004pA004pG004pU00 | | C004pC007pA004pU007pU004pA00 | |
| | 4pU004p001A004p001U004 | | 7pC004p001C007p001G004 | |
| B8 | C004pG004pG004pU004pA004pA0 | 2481 | A004p001U007p001A004pA007pC0 | 2484 |
| | 04pU007pG007pG007pA004pC004 | | 04pU007pC004pU007pG004pU007p | |
| | pA004pG004pA004pG004pU004pU | | C004pC007pA004pU007pU004pA00 | |
| | 004p001A004p001U004 | | 7pC004p001C007p001G004 | |

TABLE 6p-7

| | | SEQ | | SEQ |
|---|---|---|---|---|
| SIRNA | | ID | | ID |
| no. | Sense strand | NO: | Antisense strand | NO: |
| B9 | C007pG004pG007pU004pA007p | 2482 | A004pU007pA004pA007pC004pU007 | 2485 |
| | A004pU007pG004pG007pA004p | | pC004pU007pG004pU007pC004pC00 | |
| | C007pA004pG007pA004pG007p | | 7pA004pU007pU004pA007pC004pC0 | |
| | U004pU007pA004pU007 | | 07pG004 | |

Specific codes in the nucleotide sequences are indicated in above, e.g., Tables A and A-1.

Additional suitable dsRNAi agents targeting Lp(a), or variants thereof and synthesis thereof are also described in WO2022/098841, WO 2017/059223, WO2019/092283, WO2022/032288, WO2023/138689, WO2025/064815, WO2025/064819, and WO2025/064821, entire contents of which are incorporated herein by reference.

In some embodiments, the additional therapeutic agent includes a angiotensinogen (AGT) protein inhibitor. In some embodiments, the AGT inhibitor siRNA includes zilebesiran. In some embodiments, the AGT inhibitor siRNA includes a dsRNA including a sense strand and an antisense strand in Tables 6q-1 to 6q-11.

TABLE 6q-1

| AGT | | | SEQ ID |
|---|---|---|---|
| siRNA | Sequence (5′-3′) | Strand | NO |
| 1 | CACCAGCUUGUUUGUGAAACA | SS | 2486 |
| | UGUUUCACAAACAAGCUGGUG | AS | 2487 |
| 2 | G004p001A004p001C004pC004pA004pG004pC004pU004pU007p | SS | 2488 |
| | G004pU007pU004pU007pG004pU004pG004pA004pA004pA004pC | | |
| | 004p001A004p (GLO-0) | | |
| | U004p001G007p001U004pU004pU004pC004pA007pC004pA004p | AS | 2489 |
| | A004pA004pC007pA004pA007pG004pC007pU004pG004pG004p0 | | |
| | 01U004p001C004 | | |
| 3 | (GLS- | SS | 2490 |
| | 15) (Invab) p001C004pA004pC004pC004pA004pG004pC004pU0 | | |
| | 04pU007pG004pU007pU004pU007pG004pU004pG004pA004pA00 | | |
| | 4pA004pC004pA004p001 (Invab) | | |
| | U004p001G007p001U004pU004pU004pC004pA007pC004pA004p | AS | 2491 |
| | A004pA004pC007pA004pA007pG004pC007pU004pG004pG004p0 | | |
| | 01U004p001G004 | | |

GLO-0: delivery molecules used in the in vivo studies
GLS-15: 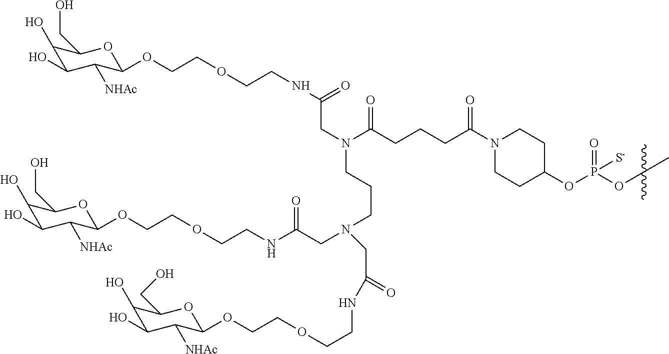 [SMILES: *OP(=O)([S-])OC1CCN(C(=O)CCCC(=O)N(CCCN(CC(=O)NCCOCCOC2OC(CO)C(O)C(O)C2NC(C)=O)CC(=O)NCCOCCOC2OC(CO)C(O)C(O)C2NC(C)=O)CC(=O)NCCOCCOC2OC(CO)C(O)C(O)C2NC(C)=O)CC1]
Imann: 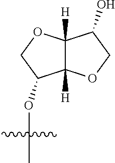 [SMILES: *O[C@@H]1CO[C@H]2[C@@H]1OC[C@H]2O]
or 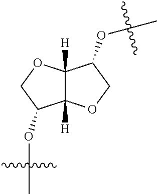 [SMILES: *O[C@@H]1CO[C@H]2[C@@H]1OC[C@H]2O*]
invab = inverted abasic

Other specific codes in the nucleotide sequences are indicated in above, e.g., Tables A and A-1.

Additional suitable additional dsRNAi agent targeting AGT, or variants thereof and synthesis thereof are also described in WO2023/088227, entire contents of which are incorporated herein by reference.

TABLE 6g-2

| | | SEQ | | SEQ |
|---|---|---|---|---|
| | | ID | | ID |
| siRNA | Sense strand (modified) | NO. | Antisense strand (modified) | NO. |
| A56 | (Invab) p001C004pA004pC004pC0 | 2492 | U004p001G007p001U004pU004pU | 2498 |
| | 04pA004pG004pC004pU004pU007p | | 004pC004pA007pC004pA004pA00 | |
| | G004pU007pU004pU007pG004pU00 | | 4pA004pC007pA004pA007pG004p | |
| | 4pG004pA004pA004pA004pC004pA | | C007pU004pG004pG004p001U004 | |
| | 004p001 (Invab) | | p001G004p | |
| A57 | (Invab) p001G004pA004pC004pC0 | 2493 | U004p001A007p001C004pU004pC | 2499 |
| | 04pU004pU004pU004pU004pC007p | | 004pA004pU007pU004pA004pG00 | |
| | U004pU007pC004pU007pA004pA00 | | 4pA004pA007pG004pA007pA004p | |
| | 4pU004pG004pA004pG004pU004pA | | A007pA004pG004pG004p001U004 | |
| | 004p001 (Invab) | | p001C004p | |
| A58 | (Invab) p001G004pA004pC004pC0 | 2494 | U004p001A007p001C004pU004pC | 2500 |
| | 04pU004pU004pU004pC004pU007p | | 004pA004pU007pU004pA004pG00 | |
| | U004pU007pC004pU007pA004pG00 | | 4pA004pA007pG004pA007pA004p | |
| | 4pC004pG004pA004pG004pU004pA | | A007pA004pG004pG004p001U004 | |
| | 004p001 (Invab) | | p001C004p | |
| A59 | (Invab) p001C004pA004pC004pC0 | 2495 | U004p001G007p001U004pU004pU | 2501 |
| | 04pA004pG004pC004pU004pU007p | | 004pC004pA007pC004pA004pA00 | |
| | G004pU007pU004pU007pG004pU00 | | 4pA004pC007pA004pA007pG004p | |
| | 4pG004pA004pA004pA004pC004pA | | C007pU004pG004pG004p001U004 | |
| | 004p001 (Invab) | | p001G004p | |
| A60 | (Invab) p001G004pC004pG004pU0 | 2496 | U004p001C007p001U004pU004pA | 2502 |
| | 04pU004pU004pC004pU004pC007p | | 004pG004pA007pC004pC004pA00 | |
| | C004pU007pU004pG007pG004pU00 | | 4pA004pG007pG004pA007pG004p | |
| | 4pC004pU004pA004pA004pG004pA | | A007pA004pA004pC004p001G004 | |
| | 004p001 (Invab) | | p001C004p | |
| A61 | (Invab) p001G004pC004pA004pA0 | 2497 | U004p001U007p001C004pG004pG | 2503 |
| | 04pA004pA004pA004pG004pA007p | | 007pU004pU044pG004pG004pA00 | |
| | A004pU007pU004pC007pC004pA00 | | 4pA004pU007pU004pC007pU004p | |
| | 4pA004pC004pC004pG004pA004pA | | U007pU004pU004pU004p001G004 | |
| | 004p001 (Invab) | | p001C004p | |

In Table 6q-2, (Invab) is an inverted abasic modification and U044 is uridine-unlocked nucleic acid. Other specific codes in the nucleotide sequences are indicated in above, e.g., Tables A and A-1.

TABLE 6q-3

| | | SEQ | | SEQ |
|---|---|---|---|---|
| | | ID | | ID |
| siRNA | Sense strand (modified) | NO. | Antisense strand (modified) | NO. |
| A62 | A007p001G004p001A007pA004pC | 2504 | U004p001U007p001A004pA007pA004p | 2516 |
| | 007pA004pA007pA004pA007pA00 | | A007pC004pC007pC004pA007pA004pU | |
| | 7pU007pU004pG007pG004pG007p | | 004pU004pU007pU004pU007pG004pU0 | |
| | U004pU007pU004pU007pA004pA0 | | 07pU004pC007pU004p001C004p001A0 | |
| | 07 | | 04 | |
| A63 | U004p001C004p001U004pC004pC | 2505 | A004p001U007p001U004pA004pG004p | 2517 |
| | 004pC004pA007pC004pC007pU00 | | A007pA004pG004pA004pA004pA004pA | |
| | 7pU007pU004pU004pC004pU004p | | 004pG004pG007pU004pG007pG004pG0 | |
| | U004pC004pU004pA004pA004pU0 | | 04pA004pG004pA004p001C004p001U0 | |
| | 04 | | 04 | |
| A64 | U004p001G004p001U004pU004pU | 2506 | U004p001U007p001U004pG004pA004p | 2518 |
| | 004pG004pC007pU004pG007pU00 | | U007pC004pA007pU007pA004pC004pA | |
| | 7pG007pU004pA004pU004pG004p | | 004pC004pA007pG004pC007pA004pA0 | |
| | A004pU004pC004pA004pA004pA0 | | 04pA004pC004pA004p001G004p001G0 | |
| | 04 | | 04 | |
| A65 | U004p001U004p001C004pC004pG | 2507 | U004p001U007p001G004pC004pA004p | 2519 |
| | 004pU004pA007pU004pA007pU00 | | U007pG004pC007pC007pA004pU004pA | |
| | 7pA007pU004pG004pG004pC004p | | 004pU004pA007pU004pA007pC004pG0 | |
| | A004pU004pG004pC004pA004pA0 | | 04pG004pA004pA004p001G004p001C0 | |
| | 04 | | 04 | |
| A66 | A004p001C004p001C004pU004pG | 2508 | U004p001A007p001U004pU004pG004p | 2520 |
| | 004pC004pA007pA004pA007pA00 | | C007pU004pC007pA007pA004pU004pU | |
| | 7pA007pU004pU004pG004pA004p | | 004pU004pU007pU004pG007pC004pA0 | |
| | G004pC004pA004pA004pU004pA0 | | 04pG004pG004pU004p001U004p001C0 | |
| | 04 | | 04 | |
| A67 | A004p001C004p001C004pU004pG | 2509 | U004p001A007p001U004pU004pG004p | 2521 |
| | 004pC004pA007pA004pA007pA00 | | C007pU004pC004pA004pA004pU004pU | |
| | 7pA007pU004pU004pG004pA004p | | 004pU004pU007pU004pG007pC004pA0 | |
| | G004pC004pA004pA004pU004pA0 | | 04pG004pG004pU004p001U004p001C0 | |
| | 04 | | 04 | |
| A68 | C004p001C004p001C004pA004pC | 2510 | U004p001U007p001C004pA004pU004p | 2522 |
| | 004pC004pU007pU004pU007pU00 | | U007pA004pG007pA007pA004pG004pA | |
| | 7pC007pU004pU004pC004pU004p | | 004pA004pA007pA004pG007pG004pU0 | |
| | A004pA004pU004pG004pA004pA0 | | 04pG004pG004pG004p001A004p001G0 | |
| | 04 | | 04 | |
| A69 | C004p001C004p001C004pA004pC | 2511 | U004p001U007p001C004pA004pU004p | 2523 |
| | 004pC004pU007pU004pU007pU00 | | U007pA004pG004pA004pA004pG004pA | |
| | 7pC007pU004pU004pC004pU004p | | 004pA004pA007pA004pG007pG004pU0 | |
| | A004pA004pU004pG004pA004pA0 | | 04pG004pG004pG004p001A004p001G0 | |
| | 04 | | 04 | |
| A70 | C004p001C004p001A004pG004pC | 2512 | U004p001U007p001U004pG004pU004p | 2524 |
| | 004pU004pU007pG004pU007pU00 | | U007pU004pC007pA007pC004pA004pA | |
| | 7pU007pG004pU004pG004pA004p | | 004pA004pC007pA004pA007pG004pC0 | |
| | A004pA004pC004pA004pA004pA0 | | 04pU004pG004pG004p001U004p001C0 | |
| | 04 | | 04 | |
| A71 | C004p001C004p001A004pG004pC | 2513 | U004p001U007p001U004pG004pU004p | 2525 |
| | 004pU004pU007pG004pU007pU00 | | U007pU004pC004pA004pC004pA004pA | |
| | 7pU007pG004pU004pG004pA004p | | 004pA004pC007pA004pA007pG004pC0 | |
| | A004pA004pC004pA004pA004pA0 | | 04pU004pG004pG004p001U004p001C0 | |
| | 04 | | 04 | |
| A72 | A004p001A004p001A004pA004pA | 2514 | U004p001U007p001G004pA004pA004p | 2526 |
| | 004pA004pG007pU004pG007pU00 | | A007pA004pG004pG004pG004pA004pA | |
| | 7pU007pC004pC004pC004pU004p | | 004pC004pA007pC004pU007pU004pU0 | |
| | U004pU004pU004pC004pA004pA0 | | 04pU004pU004pU004p001G004p001U0 | |
| | 04 | | 04 | |
| A73 | A004p001A004p001A004pA004pA | 2515 | U004p001U007p001G004pA004pA004p | 2527 |
| | 004pA004pG007pU004pG007pU00 | | A007pA004pG007pG007pG004pA004pA | |
| | 7pU007pC004pC004pC004pU004p | | 004pC004pA007pC004pU007pU004pU0 | |
| | U004pU004pU004pC004pA004pA0 | | 04pU004pU004pU004p001G004p001U0 | |
| | 04 | | 04 | |

Specific codes in the nucleotide sequences are indicated in above, e.g., Tables A and A-1.

TABLE 6q-4

| | | SEQ | | SEQ |
|---|---|---|---|---|
| | | ID | | ID |
| siRNA | Sense strand (modified) | NO. | Antisense strand (modified) | NO. |
| A74 | G004p001U004p001C004pA004pU | 2528 | U004p001G007p001U004pA004pC | 2529 |
| | 004pC004pC007pA004pC007pA00 | | 004pT1016pC004pU004pC004pA0 | |
| | 7pA007pU004pG004pA004pG004p | | 04pU004pU004pG004pU007pG004 | |
| | A004pG004pU004pA004pC004pA0 | | pG007pA004pU004pG004pA004pC | |
| | 04 | | 004p001G004p001A004 | |

In Table 6q-4, T1016 is thymidine-GNA (S-isomer). Other specific codes in the nucleotide sequences are indicated in above, e.g., Tables A and A-1.

TABLE 69-5

| | | SEQ | | SEQ |
|---|---|---|---|---|
| | | ID | | ID |
| siRNA | Sense strand (modified) | NO. | Antisense strand (modified) | NO. |
| A75 | G005p001C005*p001T005pA005pG | 2530 | A005p001A007p001G005pU007pT0 | 2533 |
| | 005pT005pC007pG005pC007pU007 | | 05pU007pT005pG005pC005*pA005 | |
| | pG007pC005*pA005pA005pA005pA | | pG005pC007pG005pA007pC005*pT | |
| | 005pC005*pT005pT005 | | 005pA005pG005pC005*p001T005p | |
| | | | 001T005 | |
| A76 | G005p001C005*p001A005pA005pA | 2531 | T005p001U007p001A005pU007pC0 | 2534 |
| | 005pG005pG007pC005*pC007pA00 | | 05*pU007pG005pC005*pT005pG00 | |
| | 7pG007pC005*pA005pG005pC005* | | 5pC005*pT005pG005pG007pC005* | |
| | pA005pG005pA005pT005pA005pA0 | | pC007pT005pT005pT005pG005pC0 | |
| | 05 | | 05*p001T005p001T005 | |
| A77 | A005p001G005p001C005*pC005*p | 2532 | T005p001U007p001A005pG007pA0 | 2535 |
| | G005pT005pU007pT005pC007pU00 | | 05pC007pC005*pA005pA005pG005 | |
| | 7pC007pC005*pT005pT005pG005p | | pG005pA005pG005pA007pA005pA0 | |
| | G005pT005pC005*pT005pA005pA0 | | 07pC005*pG005pG005pC005*pT00 | |
| | 05 | | 5p001T005p001T005 | |

TABLE 6q-6

| | | SEQ | | SEQ |
|---|---|---|---|---|
| | | ID | | ID |
| siRNA | Sense strand (modified) | NO. | Antisense strand (modified) | NO. |
| A78 | G007p001T005p001C007pT005 | 2536 | A005p001G007p001T005pU007pT005 | 2544 |
| | pC007pA005pC007pT005pU007 | | pU007pG005pC007pT005pG007pG005 | |
| | pU007pC007pC005*pA007pG00 | | pA005pA005pA007pG005pU007pG005 | |
| | 5pC007pA005pA007pA005pA00 | | pA007pG005pA007pC005*p001C005* | |
| | 7pT005pU007 | | p001C005 | |
| A79 | G007p001T005p001C007pT005 | 2537 | A005p001G007p001T005pU007pU007 | 2545 |
| | pC007pA005pC007pT005pU007 | | pU007pG005pC007pU007pG007pG005 | |
| | pU007pC007pC005*pA005pG00 | | pA005pA005pA007pG005pU007pG005 | |
| | 5pC007pA005pA005pA005pA00 | | pA007pG005pA007pC005*p001C005* | |
| | 7pC005*pU007 | | p001C005 | |
| A80 | G005p001T005p001C005*pT00 | 2538 | A005p001G007p001T005pT005pT005 | 2546 |
| | 5pC005*pA005pC007pT005pU0 | | pU007pG005pC007pU007pG005pG005 | |
| | 07pU007pC007pC005*pA005pG | | pA005pA005pA007pG005pU007pG005 | |
| | 005pC005*pA005pA005pA005p | | pA005pG005pA005pC005*p001C005* | |
| | A005pT005pT005 | | p001C005 | |
| A81 | G005p001T005p001C005*pT00 | 2539 | A005p001G007p001T005pU007pU007 | 2547 |
| | 5pC005*pA005pC007pT005pU0 | | pU007pG005pC007pU007pG005pG005 | |
| | 07pU007pC007pC005*pA005pG | | pA005pA005pA007pG005pU007pG005 | |
| | 005pC005*pA005pA005pA005p | | pA005pG005pA005pC005*pC005*p00 | |
| | A005pT005pT005 | | 1C005 | |
| A82 | G005p001T005p001C005*pT00 | 2540 | A005p001G007p001T005pU007pU007 | 2548 |
| | 5pC005*pA005pC007pT005pU0 | | pU007pG005pC007pU007pG005pG005 | |
| | 07pU007pC007pC005*pA005pG | | pA005pA005pA007pG005pU007pG005 | |
| | 005pC005*pA005pA005pA005p | | pA005pG005pA005pC005*p001C005* | |
| | A005pC005*pT005 | | p001C005 | |
| A83 | G005p001C005*p001T005pG00 | 2541 | T005p001C007p001A005pA005pA005 | 2549 |
| | 5pA005pA005pC007pA005pG00 | | pA007pA005pA007pA007pA005pT005 | |
| | 7pC007pA007pT005pT005pC00 | | pG005pC005*pU007pG005pU007pT00 | |
| | 5*pT005pT005pC005*pT005pT | | 5pC005*pA005pG005pC005*p001A00 | |
| | 005pG005pA005 | | 5p001C005 | |
| A84 | G005p001T005p001C005*pT00 | 2542 | A005p001G007p001T005pT005pT005 | 2550 |
| | 5pC005*pA005pC007pT005pU0 | | pU007pG005pC007pU007pG005pG005 | |
| | 07pU007pC007pC005*pA005pG | | pA005pA005pA007pG005pU007pG005 | |
| | 005pC005*pA005pA005pA005p | | pA005pG005pA005pC005*p001C005* | |
| | A005p001T005p001T005 | | p001C005 | |
| A85 | G005p001C005*p001T005pG00 | 2543 | T005p001C007p001A005pA005pA005 | 2551 |
| | 5pA005pA005pC007pA005pG00 | | pA007pA005pA007pA007pA005pT005 | |
| | 7pC007pA007pT005pT005pC00 | | pG005pC005*pU007pG005pU007pT00 | |
| | 5*pT005pT005pC005*pT005pT | | 5pC005*pA005pG005pC005*p001A00 | |
| | 005pG005pA005 | | 5p001C005* | |

TABLE 69-7

| | | SEQ | | SEQ |
|---|---|---|---|---|
| | | ID | | ID |
| siRNA | Sense strand (modified) | NO. | Antisense strand (modified) | NO. |
| A86 | C004pG004pA004pC004pC004pA0 | 2552 | X033U1027pU007pC004pA007pC004 | 2555 |
| | 04pG007pC007pU007pU004pG004 | | pA007pA004pA007pC004pA007pA00 | |
| | pU004pU004pU004pG004pU004pG | | 4pG007pC004pU007pG004pG007pU0 | |
| | 004p001A004p001A004 | | 04p001C007p001G004 | |
| A87 | G004pU004pG004pU004pU004pU0 | 2553 | X033U1027pU007pA004pG007pA004 | 2556 |
| | 04pC007pU007pC007pC004pU004 | | pC007pC004pA007pA004pG007pG00 | |
| | pU004pG004pG004pU004pC004pU | | 4pA007pG004pA007pA004pA007pC0 | |
| | 004p001A004p001U004 | | 04p001A007p001C004 | |
| A88 | G004pC004pA004pG004pU004pU0 | 2554 | X033U1027pA007pU004pU007pU004 | 2557 |
| | 04pG007pA007pG007pA004pA004 | | pu007pU004pG007pU004pU007pC00 | |
| | pC004pA004pA004pA004pA004pA | | 4pU007pC004pA007pA004pC007pU0 | |
| | 004p001U004p001A004 | | 04p001G007p001C004 | |

TABLE 6q-8

| | | SEQ | | SEQ |
|---|---|---|---|---|
| | | ID | | ID |
| siRNA | Sense strand (modified) | NO. | Antisense strand (modified) | NO. |
| A89 | C004p001U004p001G004pA004p | 2558 | (MeEP) - | 2570 |
| | A004pC007pA004pG007pC007pA | | U004p001C007p001A007pA004p | |
| | 007pU007pU004pU004pU004pU0 | | A007pA004pA007pA004pA004pA | |
| | 04pU004pU004pU004p001G004p | | 007pT005pG004pC004pU007pG0 | |
| | 001A004 | | 04pU007pU004pC004pA004pG00 | |
| | | | 4p001C004p001A004 | |
| A90 | U004p001U004p001U004pG004p | 2559 | (MeEP) - | 2571 |
| | U004pG007pA004pA007pA007pC | | U004p001C007p001A007pC004p | |
| | 007pA007pA004pA004pA004pA0 | | U007pU004pU007pU004pU004pU | |
| | 04pA004pG004pU004p001G004p | | 007pG004pU004pU004pU007pC0 | |
| | 001A004 | | 04pA007pC004pA004pA004pA00 | |
| | | | 4p001C004p001A004 | |
| A91 | U004p001G004p001A004pA004p | 2560 | (MeEP) - | 2572 |
| | A004pC007pA004pA007pA007pA | | U004p001G007p001G007pA004p | |
| | 007pA007pA004pG004pU004pG0 | | A007pC004pA007pC004pU004pU | |
| | 04pU004pU004pC004p001C004p | | 007pU004pU004pU004pU007pG0 | |
| | 001A004 | | 04pU007pU004pU004pC004pA00 | |
| | | | 4p001C004p001A004 | |
| A92 | A004p001A004p001A004pA004p | 2561 | (MeEP) - | 2573 |
| | G004pU007pG004pU007pU007pC | | U004p001U007p001U007pG004p | |
| | 007pC007pC004pU004pU004pU0 | | A007pA004pA007pA004pG004pG | |
| | 04pU004pC004pA004p001A004p | | 007pG004pA004pA004pC007pA0 | |
| | 001A004 | | 04pC007pU004pU004pU004pU00 | |
| | | | 4p001U004p001U004 | |
| A93 | A004p001A004p001G004pU004p | 2562 | (MeEP) - | 2574 |
| | U004pG007pA004pG007pA007pA | | U004p001C007p001A007pA004p | |
| | 007pC007pA004pA004pA004pA0 | | U007pU004pU007pU004pU004pG | |
| | 04pA004pU004pU004p001G004p | | 007pU004pU004pC004pU007pC0 | |
| | 001A004 | | 04pA007pA004pC004pU004pU00 | |
| | | | 4p001G004p001A004 | |
| A94 | A004p001G004p001A004pA004p | 2563 | (MeEP) - | 2575 |
| | C004pA007pA004pA007pA007pA | | U004p001A007p001A007pA004p | |
| | 007pU007pU004pG004pG004pG0 | | A007pC004pC007pC004pA004pA | |
| | 04pU004pU004pU004p0010004p | | 007pU004pU004pU004pU007pU0 | |
| | 001A004 | | 04pG007pU004pU004pC004pU00 | |
| | | | 4p001C004p001A004 | |
| A95 | A004p001A004p001A004pA004p | 2564 | (MeEP) - | 2576 |
| | A004pU007pU004pG007pG007pG | | U004p001A007p001U007pU004p | |
| | 007pU007pU004pU004pU004pA0 | | U004pU004pA007pA004pA004pA | |
| | 04pA004pA004pA004p001U004p | | 007pC004pC004pC004pA007pA0 | |
| | 001A004 | | 04pU007pU004pU004pU004pU00 | |
| | | | 4p001G004p001U004 | |
| A96 | G004p001G004p001U004pU004p | 2565 | (MeEP) - | 2577 |
| | U004pU007pA004pA007pA007pA | | U004p001A007p001U007pA004p | |
| | 007pU007pU004pA004pA004pA0 | | C007pU004pU007pU004pA004pA | |
| | 04pG004pU004pA004p001U004p | | 007pU004pU004pU004pU007pA0 | |
| | 001A004 | | 04pA007pA004pA004pC004pC00 | |
| | | | 4p001C004p001A004 | |
| siRNA | Sense strand (modified) | SEQ | Antisense strand (modified) | SEQ |
| | | ID | | ID |
| | | NO. | | NO. |
| A97 | G004p001U004p001U004pU004p | 2566 | (MeEP) - | 2578 |
| | U004pA007pA004pA007pA007pU | | U004p001U007p001A007pU004p | |
| | 007pU007pA004pA004pA004pG0 | | A007pC004pU007pU004pU004pA | |
| | 04pU004pA004pU004p001A004p | | 007pA004pU004pU004pU007pU0 | |
| | 001A004 | | 04pA007pA004pA004pA004pC00 | |
| | | | 4p001C004p001C004 | |
| A98 | U004p001C004p001A004pU004p | 2567 | U004p001G007p001U007pA004p | 2579 |
| | C004pC007pA004pC007pA007pA | | C007pU004pC007pU004pC004pA | |
| | 007pU007pG004pA004pG004pA0 | | 007pU004pU004pG004pU007pG0 | |
| | 04pG004pU004pA004p001C004p | | 04pG007pA004pU004pG004pA00 | |
| | 001A004 | | 4p001C004p001G004 | |
| A99 | U004p001C004p001A004pU004p | 2568 | (EP) - | 2580 |
| | C004pC007pA004pC007pA007pA | | U004p001G007p001U007pA004p | |
| | 007pU007pG004pA004pG004pA0 | | C007pU004pC007pU004pC004pA | |
| | 04pG004pU004pA004p001C004p | | 007pU004pU004pG004pU007pG0 | |
| | 001A004 | | 04pG007pA004pU004pG004pA00 | |
| | | | 4p001C004p001G004 | |
| A100 | U004p001C004p001A004pU004p | 2569 | (MeEP) - | 2581 |
| | C004pC007pA004pC007pA007pA | | U004p001G007p001U007pA004p | |
| | 007pU007pG004pA004pG004pA0 | | C007pU004pC007pU004pC004pA | |
| | 04pG004pU004pA004p001C004p | | 007pU004pU004pG004pU007pG0 | |
| | 001A004 | | 04pG007pA004pU004pG004pA00 | |
| | | | 4p001C004p001G004 | |

In Table 6q-8, (EP)-U004p001 is 5′-EPmUs (phosphate mimic linked to a 5′-terminal uracil, shown below); and (MeEP)-U004p001 is 5′-MeEPmU (mono methyl protected phosphate mimic linked to a 5′-terminal uracil, shown below). Other specific codes in the nucleotide sequences are indicated in above, e.g., Tables A and A-1.

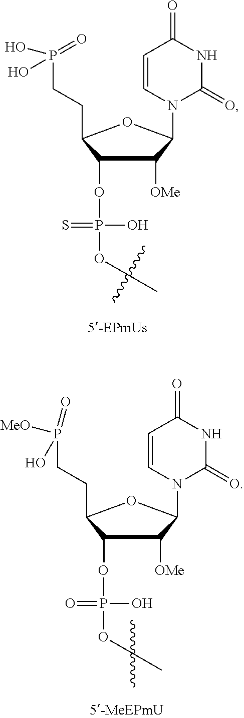

TABLE 6q-9

| | | SEQ | | SEQ |
|---|---|---|---|---|
| | | ID | | ID |
| siRNA | Sense strand (modified) | NO. | Antisense strand (modified) | NO. |
| A101 | (invdT) p001C004p001U004pG00 | 2582 | X033U1027p001U007p001G004pG0 | 2585 |
| | 4pU004pU004pC004pC004pA004p | | 07pA004pA007pU004pU007pC004p | |
| | A007pA007pA007pA004pG004pA0 | | U004pU004pU004pU004pU007pG00 | |
| | 04pA004pU004pU004pC004pC004 | | 4pG007pA004pA004pC004pA004pG | |
| | pA004pA004p001 (invdT) | | 004p001U004p001A004 | |
| A102 | (invdT) p001C004p001U004pG00 | 2583 | X033A1027p001U007p001G004pG0 | 2586 |
| | 4pU004pU004pC004pC004pA004p | | 07pA004pA007pU004pU007pC004p | |
| | A007pA007pA007pA004pG004pA0 | | U004pU004pU004pU004pU007pG00 | |
| | 04pA004pU004pU004pC004pC004 | | 4pG007pA004pA004pC004pA004pG | |
| | pA004p001 (tmU) | | 004p001U004p001A004 | |
| A103 | (invdT) p001G004p001C004pU00 | 2584 | X033U1027p001C007p001A004pA0 | 2587 |
| | 4pG004pA004pA004pC004pA004p | | 04pA004pA004pA004pA004pA004p | |
| | G007pC007pA007pC004pU004pU0 | | A004pU004pG004pC004pU007pG00 | |
| | 04pU004pU004pU004pU004pU004 | | 4pU007pU004pC004pA004pG004pC | |
| | pG004pA004p001 (invdT) | | 004p001A004p001C004 | |

In Table 6q-9, (invdT) is an inverted dT (deoxyribothymidine) with 3′-3′ linked nucleotide or 5′-5′ linked nucleotide; and (tmU) is a siRNA nucleotide with a modified sugar and a uracil base. Other specific codes in the nucleotide sequences are indicated in above, e.g., Tables A and A-1.

TABLE 6q-10

| | | SEQ | | SEQ |
|---|---|---|---|---|
| | | ID | | ID |
| siRNA | Sense strand (modified) | NO. | Antisense strand (modified) | NO. |
| A104 | C004p001C004p001U004pG004 | 2588 | U004p001G007p001A004pU007pC004 | 2592 |
| | pU004pU004pU007pA004pC007 | | pA007pU004pA007pC004pA007pC004 | |
| | pU007pG007pU007pG004pU004 | | pA007pG004pU007pA004pA007pA004 | |
| | pA004pU004pG004pA004pU004 | | pC007pA004pG007p001G004 | |
| | pC004p001A004p001 (Invab) | | | |
| A105 | U004p001G004p001A004pC004 | 2589 | A004p001C007p001A004pG007pC004 | 2593 |
| | pA004pG004pG007pU004pU007 | | pC007pU004pG007pC004pA007pU004 | |
| | pC007pA007pU007pG004pC004 | | pG007pA004pA007pC004pC007pU004 | |
| | pA004pG004pA004pC004pU004 | | pG007pU004pC007p001A004 | |
| | pG004p001U004p001 (Invab) | | | |
| A106 | C004p001G004pA004pC004pC0 | 2590 | U004p001G007p001U004pU007pU004 | 2594 |
| | 04pA004pG004pC007pU004pU0 | | pC007pA004pC007pA004pA007pA004 | |
| | 07pG007pU007pU007pU004pG0 | | pC007pA004pA007pG004pC007pU004 | |
| | 04pU004pG004pA004pA004pA0 | | pG007pG004pU007p001C004p001G00 | |
| | 04pC004p001A004p001 | | 4 | |
| | (Invab) | | | |
| A107 | A004p001A004pC004pC004pA0 | 2591 | U004p001G007p001U004pU007pU004 | 2595 |
| | 04pG004pC007pU004pU007pG0 | | pC007pA004pC007pA004pA007pA004 | |
| | 07pU007pU007pU004pG004pU0 | | pC007pA004pA007pG004pC007pU004 | |
| | 04pG004pA004pA004pA004pC0 | | pG007pG004pU007pU004p001G004p0 | |
| | 04p001A004p001 (Invab) | | 01G004 | |

In Table 6q-11, (Invab) is inverted abasic deoxyribose. Specific codes in the nucleotide sequences are indicated in above, e.g., Tables A and A-1.

TABLE 6q-11

| | | SEQ | | SEQ |
|---|---|---|---|---|
| | | ID | | ID |
| siRNA | Sense strand (modified) | NO. | Antisense strand (modified) | NO. |
| A108 | G004p001A004p001C004pC004pA | 2596 | U004p001G007p001U004pU004pU | 2598 |
| | 004pG004pC004pU004pU007pG00 | | 004pC004pA007pC004pA004pA00 | |
| | 4pU007pU004pU007pG004pU004p | | 4pA004pC007pA004pA007pG004p | |
| | G004pA004pA004pA004pC004p00 | | C007pU004pG004pG004p001U004 | |
| | 1A004p | | p001C004 | |
| A109 | p001 (Invab) p001C004pA004pC0 | 2597 | U004p001G007p001U004pU004pU | 2599 |
| | 04pC004pA004pG004pC004pU004 | | 004pC004pA007pC004pA004pA00 | |
| | pU007pG004pU007pU004pU007pG | | 4pA004pC007pA004pA007pG004p | |
| | 004pU004pG004pA004pA004pA00 | | C007pU004pG004pG004p001U004 | |
| | 4pC004pA004p001 (Invab) | | p001G004 | |

In Table 6q-11, (Invab) is inverted abasic deoxyribose. Specific codes in the nucleotide sequences are indicated in above, e.g., Tables A and A-1.

Additional suitable additional dsRNAi agent targeting AGT, or variants thereof and synthesis thereof are also described in WO2023088227, WO2015179724, WO2021096763, WO2023278576, CN114763547, CN117448322, WO2024013334, WO2024031101, WO2024187193, WO2023056446, WO2023192630, WO2014018930, WO2017062816, WO2022109139, WO2024149282, WO2022232650, CN118324834, WO2005116250, WO2013173637, WO2016196111, WO2020191243 and WO2023014765, entire contents of which are incorporated herein by reference.

### Pharmaceutical Compositions

Also provided herein is a pharmaceutical composition (“composition”) including the dsRNAi agents described herein (including all embodiments (e.g., Tables, Figures and Examples, and etc.) and a pharmaceutically acceptable carrier or excipient.

### Formulation

The pharmaceutical composition may be prepared and administered in a wide variety of dosage formulations. The dsRNAi agents described herein may be administered orally, rectally, or by injection (e.g. intravenously, intramuscularly, intracutaneously, subcutaneously, intraduodenally, or intraperitoneally). In certain embodiments, the dsRNAi agents are formulated for injection (e.g. intravenously, intramuscularly, intracutaneously, subcutaneously, intraduodenally, or intraperitoneally). In certain embodiments, the dsRNAi agents are administered subcutaneously. In certain embodiments, the dsRNAi agents are administered intravenously.

For preparing pharmaceutical compositions from the dsRNAi agents described herein, the pharmaceutically acceptable carriers or excipients may be liquid. Particularly, when parenteral application (e.g., subcutaneously or intravenously administered) is needed or desired, particularly suitable admixtures for the compounds included in the pharmaceutical composition may be injectable, sterile solutions, oily or aqueous solutions, as well as suspensions, emulsions, or implants, including suppositories. In certain aspects, for parenteral injection (e.g., subcutaneous or intravenous administration), liquid form preparations may include solutions (e.g., aqueous polyethylene glycol solution), suspensions, emulsions (e.g., water/propylene glycol solutions), aqueous or crystalline compositions, liposomal formulations, and micellar formulations. In some embodiments, the dsRNAi agent(s) or any combinations thereof that are described herein may be formulated in a sterile solution including water.

The pharmaceutically acceptable carriers or excipients can include buffers to adjust the pH to a desirable range for subcutaneous or intravenous use. In some embodiments, the pH of the pharmaceutical composition including the dsRNAi agents described herein may range from about 6.0 to about 8.0, from about 6.5 to 7.5, or from about 6.8 to 7.2. In some embodiments, the pH of the pharmaceutical compositions from the dsRNAi agents described herein may be about 6.8, about 6.9, about 7.0, about 7.1, or about 7.2. In some embodiments, the pH of the pharmaceutical composition from the dsRNAi agents described herein may be about 6.8. In some embodiments, the pH of the pharmaceutical composition from the dsRNAi agents described herein may be about 6.9. In some embodiments, the pH of the pharmaceutical composition from the dsRNAi agents described herein may be about 7.0. In some embodiments, the pH of the pharmaceutical composition from the dsRNAi agents described herein may be about 7.1. In some embodiments, the pH of the pharmaceutical composition from the dsRNAi agents described herein may be about 7.2.

In some embodiments, the liquid formulation for subcutaneous or intravenous use may include an acid (e.g., proton donor) or a base (e.g., hydroxide). In some embodiments, the liquid formulation of the pharmaceutical composition including the dsRNAi agents described herein may contain the phosphoric acid and/or sodium hydroxide. In some embodiments, the liquid formulation for subcutaneous or intravenous use may include a buffer solution containing acetate, citrate, prolamine, carbonate, phosphate, borate, sulfate, or any combination thereof, for example, the buffer solution is phosphate buffered saline (PBS). In some embodiments, the buffer solution may further include an agent to control the osmolarity, for example, proteins, peptides, amino acids, non-metabolized polymers, vitamins, ions, sugars, metabolites, organic acids, lipids, or salts (e.g., sodium chloride or potassium chloride).

In some embodiments, pharmaceutical compositions containing dsRNAi agent described herein include additional components to aid in delivery, stability, efficacy, or reduction of immunogenicity.

### Effective Dosages

The pharmaceutical composition may include compositions wherein the active ingredient dsRNAi agent is contained in a therapeutically effective amount, i.e., in an amount effective to achieve its intended purpose (e.g., gene-silencing (e.g., inhibiting, downregulating, or suppressing of the gene) the expression of HMGCR in a subject, or lowering LDL-C level in a subject). The actual amount effective for a particular application will depend, inter aim, on the condition being treated.

The pharmaceutical compositions described herein typically include a therapeutically effective amount of dsRNAi agents described herein. In some embodiments, a therapeutically effective amount of the dsRNAi agent targeting the HMGCR (e.g., human HMGCR) can reduce HMGCR mRNA levels in a treated cell or subject by at least about 10, about 15, about 20, about 25, about 30, about 35, about 40, about 45, about 50, about 55, about 60, about 65, about 70, about 75, 80, about 85, about 90 or 95% compared to non-treated or control cell or subject. In some embodiments, a therapeutically effective amount of an RNAi agent targeting HMGCR can reduce HMGCR protein levels in a treated cell or subject by at least about 10%, about 15%, about 20%, about 25%, about 30%, about 35%, about 40%, about 45%, about 50%, about 55%, about 60%, about 65%, about 70%, about 75%, about 80%, about 85, about 90% or about 95% compared to non-treated or control cell or subject. In some embodiments, a therapeutically effective amount of an RNAi agent targeting HMGCR can reduce HMGCR protein levels in a treated cell or subject by at least about 40%, about 45%, about 50%, about 55%, about 60%, about 65%, about 70%, about 75%, about 80%, about 85, or about 90% compared to non-treated or control cell or subject.

A given clinical treatment (e.g., lowering LCL-C level in blood or serum of a subject) is considered effective where there is at least about 10%, about 15%, about 20%, about 25%, about 30%, about 35%, about 40%, about 45%, about 50%, about 55%, about 60%, about 65%, about 70%, about 75%, about 80%, about 85%, about 90% or about 95% reduction in a measurable parameter associated with a disease or disorder. In some embodiments, the given clinical treatment (e.g., lowering LCL-C level in blood or serum of a subject) is considered effective where there is at least about 40%, about 45%, about 50%, about 55%, about 60%, about 65%, about 70%, about 75%, about 80%, about 85%, or about 90% reduction in a measurable parameter associated with a disease or disorder. In some embodiments, therapeutically effective amount of a dsRNAi agent for the treatment of that disease or disorder (e.g., lowering LCL-C level in blood or serum of a subject) is the amount necessary to effect at least about 10%, about 15%, about 20%, about 25%, about 30%, about 35%, about 40%, about 45%, about 50%, about 55%, about 60%, about 65%, about 70%, about 75%, about 80%, about 85%, about 90% or 95% reduction, respectively, in that parameter. In some embodiments, therapeutically effective amount of a dsRNAi agent for the treatment of that disease or disorder (e.g., lowering LCL-C level in blood or serum of a subject) is the amount necessary to effect at least about 40%, about 45%, about 50%, about 55%, about 60%, about 65%, about 70%, about 75%, about 80%, about 85%, or about 90%, respectively, in that parameter.

In certain aspects, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The dosage and frequency (single or multiple doses) of compounds administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Dosage amounts and intervals can be adjusted individually to provide levels of the administered dsRNAi agents that is effective for the particular clinical indication being treated. This will provide a therapeutic regimen that is commensurate with the severity of the individual's disease state.

The effective prophylactic or therapeutic treatment regimen as described herein can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

### Toxicity

The ratio between toxicity and therapeutic effect for the dsRNAi agent is its therapeutic index and can be expressed as the ratio between LD50 (the amount of compound lethal in 50% of the population) and ED50 (the amount of compound effective in 50% of the population). The dsRNAi agents that exhibit high therapeutic indices are preferred. Therapeutic index data obtained from cell culture assays and/or animal studies can be used in formulating a range of dosages for use in humans. The dosage of such dsRNAi agents preferably lies within a range of plasma concentrations that include the ED50 with little or no toxicity. The dosage may vary within this range depending upon the dosage form employed and the route of administration utilized. The exact formulation, route of administration, and dosage may also be chosen by the individual physician in view of the patient's condition and the particular method in which the dsRNAi agents are used.

### Combination

In certain aspects, pharmaceutical compositions may be formulated to administering the dsRNAi agent as described herein for the purpose of a combination with one or more additional therapeutic agents (second agents) that has been used or proved to be useful in treating a disorder or disease (e.g., HMGCR-associated disorder or disease, ASCVD, or a disorder of lipid metabolism) in a subject. In some embodiments, the additional agents subjected to the combination therapy may induce, promote, or facilitate gene silencing (e.g., inhibiting, downregulating, or suppressing of the gene) of the HMGCR in a subject.

In certain aspects, one or more additional therapeutic agents may be administered at the same time and/or in the same combination, e.g., parenterally, subcutaneously or intravenously, or the additional therapeutic agent can be administered as part of a separate composition or at separate times and/or by another method known in the art or described herein. For example, simultaneous administration may take place in a single pharmaceutical composition with two or more active ingredients, or by simultaneously administering two or more pharmaceutical compositions that are formulated independently. Sequential administration may take place by administrating one active ingredient (e.g., HMGCR dsRNAi agent) at one time point (first administration) and by administering other components (second administration) at a different time point, e.g., which is within 1 hour to 12 hours, or 1 to 14 days after the first administration. In some embodiments, sequential administration may take place by administrating one active ingredient (e.g., additional therapeutic agent, or inclisiran) at one time point (first administration) and by administering HMGCR dsRNAi agent (second administration) at a different time point, e.g., which is within 1 hour to 12 hours, or 1 to 14 days after the first administration.

In some embodiments, the dsRNAi agent and one or more additional therapeutic agents may be formulated (e.g., pre-mixed) in one syringe. In some embodiments, the dsRNAi agent and one or more additional therapeutic agents may be formulated in separate syringes in a single device and administered via a single device (e.g., through a single cannula, tube, or needle, or other injection device) such that the dsRNA agent and the additional therapeutic agents are mixed prior to administration. In some embodiments, the dsRNAi agent and one or more additional therapeutic agents may be formulated in separate syringes and administered via separate devices (e.g., through double or multiple cannulas, tubes, or needles, or other injection devices). In some embodiments, the dsRNAi agent and one or more additional therapeutic agents may be formulated in separate syringes and administered separately from a single device (e.g., through a single cannula, tube, or needle, or other injection device). In some embodiments, a syringe containing the dsRNAi agent and one or more separate syringes containing the additional therapeutic agents respectively may be accompanied with a single device of a co-package. In some embodiments, a syringe containing the dsRNAi agent and one or more separate syringes containing the additional therapeutic agents respectively may be accompanied with two devices of a co-package.

In some embodiments, the dsRNAi agent and inclisiran may be administered in a single formulation or pharmaceutical composition. In some embodiments, two separate pharmaceutical compositions may be formulated, and a first composition includes the dsRNAi agent as described herein (HMGCR targeting dsRNAi agent) and the second composition includes at least one of the additional therapeutic agents (second agents) described herein as active pharmaceutical ingredient. In some embodiments, the two separate pharmaceutical compositions may be administered simultaneously. In some embodiments, the two separate pharmaceutical compositions may be administered subsequently, e.g., by administering the first composition (e.g., HMGCR dsRNAi agent) and then administering the second composition, or by administering the second composition and then administering the first composition thereafter.

In some embodiments, the dsRNAi agent and inclisiran may be administered in a single formulation or pharmaceutical composition. In some embodiments, two separate pharmaceutical compositions may be formulated, and a first composition includes the dsRNAi agent as described herein (HMGCR targeting dsRNAi agent) and the second composition includes inclisiran as active pharmaceutical ingredient. In some embodiments, the two separate pharmaceutical compositions may be administered simultaneously. In some embodiments, the two separate pharmaceutical compositions may be administered subsequently, e.g., by administering the first composition (e.g., HMGCR dsRNAi agent) and then administering the second composition (e.g., inclisiran), or by administering the second composition and then administering the first composition thereafter.

### Methods of Use

The methods and compositions of the present disclosure, e.g., the methods and HMGCR RNAi agent compositions, can be used in any appropriate dosage and/or composition described herein or known in the art, as well as with any suitable route of administration described herein or known in the art. Any aspects or embodiments disclosed herein that are not mutually exclusive can be combined.

In an aspect, the disclosure provides a method of or a use for inhibiting expression of 3-hydroxy-3-methylglutaryl-CoA reductase (HMGCR) in a subject. The method or the use includes administering to the subject the dsRNAi agent or a pharmaceutical composition as described herein. In some embodiments, the method includes administering to the subject a therapeutically effective amount of the dsRNAi agent or a pharmaceutical composition as described herein.

In an aspect, the disclosure provides a method of, or a use for inhibiting expression of 3-hydroxy-3-methylglutaryl-CoA reductase (HMGCR) in a subject. The method or the use includes administering to the subject the dsRNAi agent including the siRNA selected from Tables 1-4, or a pharmaceutical composition thereof. In some embodiments, the method includes administering to the subject a therapeutically effective amount of the dsRNAi agent including the siRNA selected from Tables 1-4, or a pharmaceutical composition thereof.

In certain aspects, the disclosure provides a method of, or for a use for treating or preventing the HMGCR-associated disorder or disease by reduction in HMGCR expression. The method or the use includes administering to the subject the dsRNAi agent including the siRNA selected from Tables 1-4, or a pharmaceutical composition thereof. In some embodiments, the method includes administering to the subject a therapeutically effective amount of the dsRNAi agent including the siRNA selected from Tables 1-4, or a pharmaceutical composition thereof. The level of HMGCR may be measured or detected in a sample (e.g., a blood, serum, or liver tissue) from the subject. In certain aspects, the method may include treating or preventing one or more symptoms in the subject having the HMGCR-associated disorder or disease. In some embodiments, the methods may decrease HMGCR protein accumulation.

Example HMGCR-associated disorders or diseases include acquired or inherited disorders of lipid metabolism. In some embodiments, HMGCR-associated disorders or diseases include any disorder associated with or caused by a disturbance in lipid metabolism, e.g., abnormal elevation of levels of any or all lipids and/or lipoproteins in the blood or a condition that can lead to abnormal elevation of levels of any or all lipids and/or lipoproteins in the blood, such as a hyperlipidemia, and other forms of lipid imbalance such as hypercholesterolemia, hypertriglyceridemia, mixed hyperlipidemia, primary hyperlipidemia, heterozygous familiar hypercholesterolemia (HeFH), homozygous familiar hypercholesterolemia (HoFH), as well as the pathological conditions associated with these disorders, e.g., congestive heart disease (CHD) and atherosclerosis. In some embodiments, the method is for treating hyperlipidemia. In some embodiments, the method is for treating hypercholesterolemia. In some embodiments, the method is for treating hypertriglyceridemia. In some embodiments, the method is for treating mixed hyperlipidemia. In some embodiments, the method is for treating primary hyperlipidemia. In some embodiments, the method is for treating heterozygous familiar hypercholesterolemia (HeFH). In some embodiments, the method is for treating homozygous familiar hypercholesterolemia (HoFH). In some embodiments, the method is for treating congestive heart disease (CHD). In some embodiments, the method is for treating atherosclerosis.

In an aspect, the disclosure also provides a method of, or a use for lowering a level of low-density lipoprotein cholesterol (LDL-C) in a subject. The method or the use includes administering to the subject the dsRNAi agent including the siRNA selected from Tables 1-4, or a pharmaceutical composition thereof. In some embodiments, the method includes administering to the subject a therapeutically effective amount of the dsRNAi agent including the siRNA selected from Tables 1-4, or a pharmaceutical composition thereof. In some embodiments, the level of low-density lipoprotein cholesterol (LDL-C) can be measured in a blood vessel of a subject. The method includes administering to the subject the dsRNAi agent or a pharmaceutical composition as described herein. In some embodiments, the method includes administering to the subject a therapeutically effective amount of the dsRNAi agent or a pharmaceutical composition as described herein.

In an aspect, the disclosure provides a method of, or a use for treating or preventing hyperlipidemia in a subject. The method or the use includes administering to the subject the dsRNAi agent including the siRNA selected from Tables 1-4, or a pharmaceutical composition thereof. In some embodiments, the method includes administering to the subject a therapeutically effective amount of the dsRNAi agent including the siRNA selected from Tables 1-4, or a pharmaceutical composition thereof. The method includes administering to the subject the dsRNAi agent or a pharmaceutical composition as described herein. In some embodiments, the method includes administering to the subject a therapeutically effective amount of the dsRNAi agent or a pharmaceutical composition as described herein.

In an aspect, the disclosure provides a method of, or a use for treating or preventing atherosclerotic cardiovascular disease (ASCVD) in a subject. The method includes administering to the subject the dsRNAi agent or a pharmaceutical composition as described herein. In some embodiments, the method includes administering to the subject a therapeutically effective amount of the dsRNAi agent or a pharmaceutical composition as described herein. In some embodiments, the method or the use includes administering to the subject the dsRNAi agent including the siRNA selected from Tables 1-4, or a pharmaceutical composition thereof. In some embodiments, the method includes administering to the subject a therapeutically effective amount of the dsRNAi agent including the siRNA selected from Tables 1-4, or a pharmaceutical composition thereof.

In certain aspects, for the methods described above, administration of the dsRNAi agent or the pharmaceutical composition may be, but not limited to, subcutaneous, intravenous, intramuscular, intraocular, intrabronchial, intrapleural, intraperitoneal, intraarterial, lymphatic, cerebrospinal, and any combinations thereof. In some embodiments, the dsRNAi agent or the pharmaceutical composition may be administered subcutaneously or intravenously.

In some embodiments, the dsRNAi agent is be delivered locally (e.g., to the site of the disease, such as a liver) so that levels of HMGCR outside the diseased areas can be maintained as close to normal as possible. In some embodiments, the level of HMGCR in the body can be modulated such that it is low enough to improve the disease state, but not so low that organ pathology occurs.

In some embodiments, the administration is via a depot injection that can release the dsRNAi agent in a consistent way over a prolonged time period, so as to reduce the frequency of dosing needed to obtain a desired effect, e.g., a desired inhibition of HMGCR, or a therapeutic or prophylactic effect and/or to provide more consistent serum concentrations of the therapeutic agent. In some embodiments, the administration is via a pump, e.g., external pump or a surgically implanted pump. For example, the pump is a subcutaneously implanted osmotic pump or an infusion pump for intravenous, subcutaneous, arterial, or epidural infusions. In some embodiments, the pump is a surgically implanted pump that delivers the dsRNAi agent described herein directly or closely to the liver.

In certain aspects, the methods described above further include administering to the subject an additional therapeutic agent. Combinations of two or more therapeutic agents (e.g., dsRNAi agent targeting HMGCR and an additional therapeutic agent) of sequential, separate and simultaneous administration are possible, preferably such that the combination of the therapeutic agents (e.g., dsRNAi agent targeting HMGCR and an additional therapeutic agent) show a joint therapeutic effect that exceeds the effect found when each single agent is used independently (e.g., at an interval so long that mutual or synergetic effect from combinations of therapeutic agents is not found).

Alternatively, in an aspect, the disclosure provides a method of, or use for treating or preventing the HMGCR-associated disorder or disease by reduction in HMGCR expression. The method includes administering to the subject a combination described above (i.e., composition including the combination of the dsRNAi agent as described herein and an additional therapeutic agent). In some embodiments, the method or the use includes administering to the subject the dsRNAi agent including the siRNA selected from Tables 1-4 and the and an additional therapeutic agent, or a pharmaceutical composition including the combination thereof. In some embodiments, the method includes administering to the subject a therapeutically effective amount of the dsRNAi agent including the siRNA selected from Tables 1-4 and the and an additional therapeutic agent, or a pharmaceutical composition including the combination thereof.

In an aspect, the disclosure provides a method of, or a use for treating or preventing hyperlipidemia in a subject and the method includes administering to the subject the combination described above. The method or the use includes administering to the subject the dsRNAi agent including the siRNA selected from Tables 1-4 and the and an additional therapeutic agent, or a pharmaceutical composition including the combination thereof. In some embodiments, the method includes administering to the subject a therapeutically effective amount of the dsRNAi agent including the siRNA selected from Tables 1-4 and the and an additional therapeutic agent, or a pharmaceutical composition including the combination thereof.

In an aspect, the disclosure provides a method of, or a use for treating or preventing atherosclerotic cardiovascular disease (ASCVD) in a subject and the method includes administering to the subject the combination described above. The method or the use includes administering to the subject the dsRNAi agent including the siRNA selected from Tables 1-4 and the and an additional therapeutic agent, or a pharmaceutical composition including the combination thereof. In some embodiments, the method includes administering to the subject a therapeutically effective amount of the dsRNAi agent including the siRNA selected from Tables 1-4 and the and an additional therapeutic agent, or a pharmaceutical composition including the combination thereof.

The additional therapeutic agents suitable for the combination with the dsRNAi agents described herein may include a drug or agent that has been used or proven to be useful in treating a disorder of lipid metabolism (e.g., high cholesterol). In certain aspects, pharmaceutical compositions are formulated to administering the dsRNAi agents as described herein for the purpose of a combination with one or more additional therapeutic agents that has been used or proved to be useful in lowering LDL-C in a subject. In certain aspects, the additional therapeutic agent may suitably include selected from a HMGCR small molecule inhibitor (e.g., statins), a proprotein convertase subtilisin kexin 9 (PCSK9) inhibitor, a fibrate, a bile acid sequestrant, niacin, an antiplatelet agent, an angiotensin converting enzyme inhibitor, an angiotensin II receptor antagonist, an acyl-CoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

In some embodiments, the additional therapeutic agent suitable for the combination with the dsRNAi agents described herein may include a lysophosphatidic acid (LPA) receptor inhibitor. In some embodiments, the additional therapeutic agent may include an angiotensinogen (AGT) inhibitor. In some embodiments, the additional therapeutic agent may include bile sequestering agents (e.g., cholestyramin E). In some embodiments, the additional therapeutic agent includes VLDL secretion inhibitors (e.g., niacin). In some embodiments, the additional therapeutic agent includes lipophilic antioxidants (e.g., Probucol). In some embodiments, the additional therapeutic agent includes acyl-CoA cholesterol acyl transferase inhibitors. In some embodiments, the additional therapeutic agent includes farnesoid X receptor antagonists. In some embodiments, the additional therapeutic agent includes sterol regulatory binding protein cleavage activating protein (SCAP) activators. In some embodiments, the additional therapeutic agent includes microsomal triglyceride transfer protein (MTP) inhibitors. In some embodiments, the additional therapeutic agent includes inhibitors to apolipoproteins (e.g., ApoA1, ApoB, ApoC3, ApoD, ApoE, ApoF, or ApoM). In some embodiments, the additional therapeutic agent includes and therapeutic antibodies against HMGCR. In some embodiments, the additional therapeutic agents may also include agents that raise high density lipoprotein (HDL). In some embodiments, the additional therapeutic agent includes such as cholesteryl ester transfer protein (CETP) inhibitors. In some embodiments, the additional therapeutic agents may also include dietary supplements, e.g., fish oil, and omega-3 oils. In some embodiments, the additional therapeutic agents do not include a HMGCR small molecule inhibitor (e.g., statins).

In some embodiments, the additional therapeutic agent or the second agent includes the PCSK9 inhibitor. In some embodiments, the PCSK9 inhibitor may be a small molecule, antibody, peptide, or a therapeutic RNA interference agent (e.g., siRNA). In some embodiments, the additional therapeutic agent includes a second dsRNAi agent (e.g., siRNA) targeting PCSK9. In some embodiments, the additional therapeutic agent particularly includes inclisiran (e.g., Leqvio®). In some embodiments, the second dsRNAi agent includes at least one of PCSK9 siRNA as described in Tables 6a to 6o or a variant thereof.

In some embodiments, the additional therapeutic agent or the second agent is an ASO agent. In some embodiments, the additional therapeutic agent includes the apoprotein (e.g., ApoA1, ApoC3, or ApoE) inhibitor. In some embodiments, the ApoA1 inhibitor may be a small molecule, antibody, peptide, or a therapeutic oligonucleotides (e.g., antisense oligonucleotide or “ASO”). In some embodiments, the additional therapeutic agent includes an antisense oligonucleotide targeting ApoA1 or ApoC3. In some embodiments, the ASO agent targets LPA gene. In some embodiments, the additional therapeutic agent particularly includes pelacarsen. In some embodiments, the additional therapeutic agent particularly includes olezarsen.

In some embodiments, the additional therapeutic agent includes lipoprotein (a) (LPA), or otherwise Apo(a) inhibitor. In some embodiments, the LPA (ApoA) inhibitor siRNA includes at least one of LPA siRNA as described in Tables 6p or a variant thereof.

In some embodiments, the additional therapeutic agent includes angiotensinogen (AGT) protein inhibitor. In some embodiments, the AGT inhibitor siRNA includes at least one of AGT siRNA as described in Tables 6q or a variant thereof. In some embodiments, the AGT inhibitor siRNA includes zilebesiran.

The dsRNAi agent as described herein and an additional therapeutic agent can be administered in any order, simultaneously or sequentially, or in multiple doses over time. Simultaneous administration may take place in the form of one fixed combination with two or more therapeutic agents (e.g., API), or by simultaneously administering two or more therapeutic agents (e.g., API) that are formulated independently. Sequential administration may be administration of one (first) therapeutic agent of a combination at one time point, other (second) therapeutic agent at a different time point, e.g., in a chronically staggered manner. In some embodiments, the sequential administration of the combination is scheduled to provide more efficiency than the single compounds administered independently (especially showing synergism). Separate administration may be administration of the components of the combination independently of each other at different time points, for example, meaning that the components are administered such that no overlap of measurable blood levels of both compounds are present in an overlapping manner (at the same time).

In some embodiments, the dsRNAi agent or the pharmaceutical composition as described herein and the additional therapeutic agent are administered simultaneously. The additional therapeutic agents may be administered at the same time and/or in the same composition, e.g., parenterally, subcutaneously or intravenously. For example, simultaneous administration may take place in a single pharmaceutical composition with two or more therapeutic agents (e.g., API), or by simultaneously administering two or more pharmaceutical compositions that are formulated independently.

In some embodiments, the dsRNAi agent or the pharmaceutical composition as described herein and the additional therapeutic agent are administered subsequently, e.g., the additional therapeutic agent can be administered as part of a separate composition or at separate times and/or by another method known in the art or described herein. In some embodiments, sequential administration may take place by administrating one active ingredient (e.g., HMGCR dsRNAi agent) at one time point (first administration) and by administering other components (second administration) at a different time point, e.g., which is within 1 hour to 12 hours, or 1 to 14 days after the first administration, while the first administration is at least in effect. In some embodiments, sequential administration may take place by administrating one active ingredient (e.g., additional therapeutic agent, or inclisiran) at one time point (first administration) and by administering HMGCR dsRNAi agent (second administration) at a different time point, e.g., which is within 1 hour to 12 hours, or 1 to 14 days after the first administration, while the first administration is at least in effect.

Devices (e.g., depot injection, pump, or implants) described above for the administration may be independently used for the additional therapeutic agent. In some embodiments, the dsRNAi agent or the pharmaceutical composition and the additional therapeutic agent are administered subcutaneously. In some embodiments, the dsRNAi agent or the pharmaceutical composition and the additional therapeutic agent are administered intravenously. In some embodiments, the dsRNAi agent or the pharmaceutical composition and the additional therapeutic agent are administered subcutaneously and intravenously, respectively.

In some embodiments, the subject is a human. In some embodiments, the subject has or is diagnosed with hypercholesterolemia. In some embodiments, the subject has or is diagnosed with HMGCR-associated disorder or disease.

In some embodiments, the subject after treatment (e.g., after administering the dsRNAi agent or the pharmaceutical composition described herein) does not have a muscle side effect. In some embodiments, the subject the after treatment (e.g., after administering the dsRNAi agent or the pharmaceutical composition described herein) does not have a skeletal muscle side effect (e.g., muscle AE).

In certain aspects, provided is a method of reducing the risk of a major adverse cardiovascular event in a subject, comprising administering to the subject the dsRNAi agent as described herein or a pharmaceutically acceptable salt thereof, the pharmaceutical composition as described herein, the combination as described herein, or the pharmaceutical composition comprising the combination as described herein.

In some embodiments, the major adverse cardiovascular event is cardiovascular death, non-fatal myocardial infarction, non-fatal ischemic stroke, or urgent coronary revascularization.

In some embodiments, the subject has an established cardiovascular disease.

In some embodiments, the subject has not experienced a major atherosclerotic cardiovascular disease (ASCVD) event.

All methods described herein can be performed in any suitable order unless otherwise indicated herein or otherwise clearly contradicted by context. The use of any and all examples, or example language (e.g. “such as”) provided herein is intended merely to better illuminate the invention and does not pose a limitation on the scope of the invention otherwise claimed.

### Kit

In an aspect, provided is a kit including the dsRNAi agent or the pharmaceutical composition as described herein. In certain aspects, the kit further includes one or more applicator.

In certain aspects, the kit may include a suitable container containing a pharmaceutical composition (e.g., HMGCR dsRNAi agent) as described herein. In some embodiments, the container may be a vial or a pre-filled syringe. In certain aspects, the kit may include a suitable applicator (e.g., an injection device) for parenterally (e.g., subcutaneously or intravenously) administering the pharmaceutical composition (e.g., HMGCR dsRNAi agent) as described herein. In some embodiments, the kit includes a syringe as an applicator and optionally include a needle (e.g., with or without cannula). In some embodiments, the kit includes a pre-filled syringe containing the pharmaceutical composition (e.g., HMGCR dsRNAi agent), optionally with a needle (e.g., with or without cannula).

The additional therapeutic agents suitable for the combination with the dsRNAi agents described herein may include a drug or agent that has been used or proven to be useful in treating a disorder of lipid metabolism (e.g., high cholesterol). In certain aspects, pharmaceutical compositions are formulated to administering the dsRNAi agents as described herein for the purpose of a combination with one or more additional therapeutic agents that has been used or proved to be useful in lowering LDL-C in a subject. In certain aspects, the additional therapeutic agent may suitably include selected from a HMGCR small molecule inhibitor (e.g., statins), a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acyl-CoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

In some embodiments, the additional therapeutic agent suitable for the combination with the dsRNAi agents described herein may include a lysophosphatidic acid (LPA) receptor inhibitor. In some embodiments, the additional therapeutic agent may include an angiotensinogen (AGT) inhibitor. In some embodiments, the additional therapeutic agent may include bile sequestering agents (e.g., cholestyramin E). In some embodiments, the additional therapeutic agent includes VLDL secretion inhibitors (e.g., niacin). In some embodiments, the additional therapeutic agent includes lipophilic antioxidants (e.g., Probucol). In some embodiments, the additional therapeutic agent includes acyl-CoA cholesterol acyl transferase inhibitors. In some embodiments, the additional therapeutic agent includes farnesoid X receptor antagonists. In some embodiments, the additional therapeutic agent includes sterol regulatory binding protein cleavage activating protein (SCAP) activators. In some embodiments, the additional therapeutic agent includes microsomal triglyceride transfer protein (MTP) inhibitors. In some embodiments, the additional therapeutic agent includes inhibitors to apolipoproteins (e.g., ApoA1, ApoB, ApoC3, ApoD, ApoE, ApoF, or ApoM). In some embodiments, the additional therapeutic agent includes and therapeutic antibodies against HMGCR. In some embodiments, the additional therapeutic agents may also include agents that raise high density lipoprotein (HDL). In some embodiments, the additional therapeutic agent includes such as cholesteryl ester transfer protein (CETP) inhibitors. In some embodiments, the additional therapeutic agents may also include dietary supplements, e.g., fish oil, and omega-3 oils. In some embodiments, the additional therapeutic agents do not include a HMGCR small molecule inhibitor (e.g., statins).

In certain aspects, the additional therapeutic agent or the second agent is a second dsRNAi agent. In some embodiments, the second dsRNAi agent is a dsRNAi agent that targets one or more of the genes selected from the group consisting of PCSK9, LPA, AGT, ACE, ACE2, AGTR1, AGTR2, ApoA1, ApoB, ApoC3, ApoD, ApoE, ApoF, ApoM, ACAT, CETP, MTTP, PPAR, IBAT, FDFT1, ERG9, SQS1, Ccl2, CCR2, CCL7, CCL8, CCL13, and CCL16. In some embodiments, the second dsRNAi agent targets PCSK9 gene. In some embodiments, the second dsRNAi agent targets LPA gene. In some embodiments, the second dsRNAi agent targets AGT gene. In some embodiments, the second dsRNAi agent targets ACE gene. In some embodiments, the second dsRNAi agent targets ACE2 gene. In some embodiments, the second dsRNAi agent targets AGTR1 gene. In some embodiments, the second dsRNAi agent targets ApoA1 gene. In some embodiments, the second dsRNAi agent targets ApoB gene. In some embodiments, the second dsRNAi agent targets ApoC3 gene. In some embodiments, the second dsRNAi agent targets ApoD gene. In some embodiments, the second dsRNAi agent targets ApoE gene. In some embodiments, the second dsRNAi agent targets ApoF gene. In some embodiments, the second dsRNAi agent targets ApoM gene. In some embodiments, the second dsRNAi agent targets AGTR2 gene. In some embodiments, the second dsRNAi agent targets ACATgene. In some embodiments, the second dsRNAi agent targets CETP gene. In some embodiments, the second dsRNAi agent targets MTTP gene. In some embodiments, the second dsRNAi agent targets PPAR gene. In some embodiments, the second dsRNAi agent targets IBAT gene. In some embodiments, the second dsRNAi agent targets FDFT1 gene. In some embodiments, the second dsRNAi agent targets ERG9 gene. In some embodiments, the second dsRNAi agent targets SQS1 gene. In some embodiments, the second dsRNAi agent targets CCL2 gene. In some embodiments, the second dsRNAi agent targets CCR2 gene. In some embodiments, the second dsRNAi agent targets CCL7 gene. In some embodiments, the second dsRNAi agent targets CCL8 gene. In some embodiments, the second dsRNAi agent targets CCL13 gene. In some embodiments, the second dsRNAi agent targets CCL16 gene.

In some embodiments, the additional therapeutic agent or the second agent includes the PCSK9 inhibitor. In some embodiments, the PCSK9 inhibitor may be a small molecule, antibody, peptide, or a therapeutic RNA interference agent (e.g., siRNA). In some embodiments, the additional therapeutic agent includes a second dsRNAi agent (e.g., siRNA) targeting PCSK9. In some embodiments, the additional therapeutic agent particularly includes inclisiran (e.g., Leqvio®). In some embodiments, the second dsRNAi agent includes at least one of PCSK9 siRNA as described in Tables 6a to 6o or a variant thereof.

In some embodiments, the additional therapeutic agent or the second agent is an ASO agent. In some embodiments, the additional therapeutic agent includes the apoprotein (e.g., ApoA1, ApoC3, or ApoE) inhibitor. In some embodiments, the ApoA1 inhibitor may be a small molecule, antibody, peptide, or a therapeutic oligonucleotides (e.g., antisense oligonucleotide or “ASO”). In some embodiments, the additional therapeutic agent includes an antisense oligonucleotide targeting ApoA1 or ApoC3. In some embodiments, the ASO agent targets LPA gene. In some embodiments, the additional therapeutic agent particularly includes pelacarsen. In some embodiments, the additional therapeutic agent particularly includes olezarsen.

In some embodiments, the additional therapeutic agent includes lipoprotein (a) (LPA), or otherwise Apo(a) inhibitor. In some embodiments, the LPA (ApoA) inhibitor siRNA includes at least one of LPA siRNA as described in Tables 6p or a variant thereof.

In some embodiments, the additional therapeutic agent includes angiotensinogen (AGT) protein inhibitor. In some embodiments, the AGT inhibitor siRNA includes at least one of AGT siRNA as described in Tables 6q or a variant thereof. In some embodiments, the AGT inhibitor siRNA includes zilebesiran.

In certain aspects, the dsRNAi agent and the additional therapeutic agent may be provided in one container, e.g., a single vial or pre-filled syringe. Alternatively, the dsRNAi agent and the additional therapeutic agent may be provided separately in two or more containers, e.g., separate vials or pre-filled syringes, e.g., one container for HMGCR dsRNAi agent, compound preparation, one container for the additional therapeutics, and/or another container for carrier components. In some embodiments, the dsRNAi agent and the additional therapeutic agent may be provided in separate vials or separate pre-filled syringe.

In certain aspects, the kit may include an instruction for use, e.g., instructions for administering or determining a therapeutically effective amount of a pharmaceutical composition (e.g., HMGCR dsRNAi agent) as described herein. In some embodiments, the instruction provides information for combined administrations, e.g., how to prepare and/or administer the two or more pharmaceutical compositions, e.g., compositions including the dsRNAi agent and the additional therapeutic agent, respectively. In some embodiments, the instruction may provide instruction for simultaneous or subsequential administration as described herein.

In some embodiments, the combined administration may include using one or more needles (e.g., with or without cannula). In some embodiments, two or more separate pharmaceutical compositions may be combined and administered simultaneously. In some embodiments, two or more separate pharmaceutical compositions may be combined and administered simultaneously using a needle with two or more openings or cannulars. In some embodiments, two or more separate pharmaceutical compositions may be administered sequentially using a needle with two or more openings or cannulars. In some embodiments, two or more separate pharmaceutical compositions may be administered with separate syringes and needles.

### Embodiments (I)

Embodiment 1: A double stranded RNAi (dsRNAi) agent comprising:
(a) a sense strand comprising a nucleotide sequence selected from SEQ ID NO: 3 and an antisense strand comprising a nucleotide sequence selected from SEQ ID: 409;
(b) a sense strand comprising a nucleotide sequence selected from SEQ ID NO: 6 and an antisense strand comprising a nucleotide sequence selected from SEQ ID: 412;
(c) a sense strand comprising a nucleotide sequence selected from SEQ ID NO: 284 and an antisense strand comprising a nucleotide sequence selected from SEQ ID: 689;
(d) a sense strand comprising a nucleotide sequence selected from SEQ ID NO: 1434 and an antisense strand comprising a nucleotide sequence selected from SEQ ID: 1441;
(e) a sense strand comprising a nucleotide sequence selected from SEQ ID NO: 1435 and an antisense strand comprising a nucleotide sequence selected from SEQ ID: 1442;
(f) a sense strand comprising a nucleotide sequence selected from SEQ ID NO: 1436; and an antisense strand comprising a nucleotide sequence selected from SEQ ID: 1443;
(g) a sense strand comprising a nucleotide sequence selected from SEQ ID NO: 1437; and an antisense strand comprising a nucleotide sequence selected from SEQ ID: 1444;
(h) a sense strand comprising a nucleotide sequence selected from SEQ ID NO: 1438; and an antisense strand comprising a nucleotide sequence selected from SEQ ID: 1445;
(i) a sense strand comprising a nucleotide sequence selected from SEQ ID NO: 1439; and an antisense strand comprising a nucleotide sequence selected from SEQ ID: 1446; and
(j) a sense strand comprising a nucleotide sequence selected from SEQ ID NO: 1440; and an antisense strand comprising a nucleotide sequence selected from SEQ ID: 1447.

Embodiment 2: A double stranded RNAi (dsRNAi) agent comprising:
(i) a sense strand comprising a nucleotide sequence selected from SEQ ID Nos. 1294 to 1297 and 1448 to 1462 in Table 3;
(ii) an antisense strand forming a duplex with the sense strand and comprising a nucleotide sequence selected from SEQ ID Nos. 1298 to 1301, and 1463 to 1477 in Table 3.

Embodiment 3: The dsRNAi agent of Embodiment 1 or 2, wherein all the nucleotides in the sense strand and the antisense strand are modified nucleotides.

Embodiment 4: The dsRNAi agent of Embodiment 1 through 3, wherein each of the modified nucleotides independently comprises one or more modifications selected from a deoxy modification, a 2′-O-alkyl modification, a 2′-halo modification, a 2′-5′-linkage modification, a conformationally restricting modification, an abasic modification, a 2′-amino-modification, a 2′-O-allyl modification, 2′-C-alkyl modification, a 2′-O-alkoxyalkyl modification, a morpholino modification, a phosphoramidate modification, a modification containing a non-natural nucleobase, a modification in a tetrahydropyran, a modification containing a 1,5-anhydrohexitol, a modification containing a cyclohexenyl, a modification containing a phosphorothioate group, a modification containing a methylphosphonate group, a modification containing a 5′-phosphate, a modification to form a thermally destabilizing nucleotide, a modification containing glycol, and a 2-O-(N-methylacetamide) modification.

Embodiment 5: The dsRNAi agent of Embodiment 4, wherein each of the modified nucleotides is independently selected from GNA, 2′-O-alkoxyalkyl modified nucleotide, 2′-O-alkyl modified nucleotide, 2′-O-allyl modified nucleotide, 2′-C-allyl modified nucleotide, and 2′-halo modified nucleotide.

Embodiment 6: The dsRNAi agent of Embodiment 3, wherein all the modified nucleotides comprise a modification on a 2′ sugar ring.

Embodiment 7: The dsRNAi agent of Embodiment 6, wherein the modified nucleotides are selected from a 2′-O-alkyl modified nucleotide, a 2′-halo modified nucleotide, a 2′-deoxy modified nucleotide, and a 2′-O-alkoxyalkyl modified nucleotide.

Embodiment 8: The dsRNAi agent of any one of Embodiments 3 through 7, wherein one or more of the modified nucleotides further comprises a phosphorothioate (PS) modification.

Embodiment 9: The dsRNAi agent of any one of Embodiments 3 through 8, wherein each of the modified nucleotides comprises one or more modifications selected from 2′-O-methyl (2′-OMe) modification, 2′-fluoro (2′-F) modification, 2′-O-methoxyethyl (2′-MOE) modification, 3′-phosphorothioate (PS) modification, and 5′-vinyl-phosphonate (5′-VP) modification.

Embodiment 10: The dsRNAi agent of any one of Embodiments 1 through 9, wherein the sense strand comprises 2′-MOE modified nucleotides positioned at the 1st, 2nd, 20th, and 21st nucleotides from the 5′-end of the sense strand.

Embodiment 11: The dsRNAi agent of Embodiment 10, wherein the sense strand comprises only four 2′-MOE modified nucleotides.

Embodiment 12: The dsRNAi agent of any one of Embodiments 1 through 11, wherein the antisense strand comprises a 5′-(E)-VP group at the 1st nucleotide from 5′ end of the antisense strand.

Embodiment 13: The dsRNAi agent of any one of Embodiments 1 through 12, wherein the antisense strand comprises a 5′-(E)-VP-2′-OMe group at the 1st nucleotide from 5′ end of the antisense strand.

Embodiment 14: The dsRNAi agent of any one of Embodiments 1 and 13, wherein each of the sense strand and the antisense strand independently comprises two, three, four, five or six 2′-F modified nucleotides.

Embodiment 15: The dsRNAi agent of any one of Embodiments 1 through 14, wherein the sense strand comprises two, three, or four 2′-F modified nucleotides positioned at the 7th, 9th, 10th, and/or 11th nucleotide from 5′-end of the sense strand.

Embodiment 16: The dsRNAi agent of Embodiment 15, wherein the sense strand comprises 2′-F modified nucleotides positioned at the 7th, 9th, 10th, and 11th nucleotides from 5′-end of the sense strand.

Embodiment 17: The dsRNAi agent of Embodiment 16, wherein the sense strand comprises 2′-OMe modified nucleotides constituting the remaining positions in the sense strand.

Embodiment 18: The dsRNAi agent of any one of Embodiments 1 through 17, wherein the antisense strand comprises two, three, or four 2′-F modified nucleotides positioned at the 2nd, 6th, 14th, and/or 16th nucleotides from 5′-end of the antisense strand.

Embodiment 19: The dsRNAi agent of Embodiment 18, wherein the antisense strand comprises 2′-F modified nucleotides positioned at the 2nd, 6th, 14th, and 16th nucleotides from 5′-end of the antisense strand.

Embodiment 20: The dsRNAi agent of Embodiment 18 or 19, wherein the antisense strand comprises a GNA at the 5th nucleotide from 5′-end of the antisense strand.

Embodiment 21: The dsRNAi agent of Embodiment 19 or 20, wherein the antisense strand comprises 2′-OMe modified nucleotides constituting the remaining positions in the antisense strand.

Embodiment 22: The dsRNAi agent of any one of Embodiments 1 through 21, wherein the sense strand comprises two, three, or four 3′-(PS) modification at the 1st, 2nd, 19th and/or 20th nucleotides from 5′-end of the sense strands.

Embodiment 23: The dsRNAi agent of any one of Embodiments 1 through 22, wherein the antisense strand comprises two, three, or four 3′-(PS) modification at the 1st, 2nd, 21st and/or 22nd nucleotides from 5′-end of the antisense strands.

Embodiment 24: A double stranded RNAi (dsRNAi) agent, comprising,
(i) a sense strand comprising a nucleotide sequence of SEQ ID NO: 1294, and an antisense strand comprising a nucleotide sequence of SEQ ID NO: 1298;
(ii) a sense strand comprising a nucleotide sequence of SEQ ID NO: 1295, and an antisense strand comprising a nucleotide sequence of SEQ ID NO: 1299;
(iii) a sense strand comprising a nucleotide sequence of SEQ ID NO: 1296, and an antisense strand comprising a nucleotide sequence of SEQ ID NO: 1300; or
(iv) a sense strand comprising a nucleotide sequence of SEQ ID NO: 1297, and an antisense strand comprising a nucleotide sequence of SEQ ID NO: 1301.

Embodiment 25: The dsRNAi agent of any one of Embodiments 1 through 24, further comprising a ligand.

Embodiment 26: The dsRNAi agent of Embodiment 25, wherein the ligand comprises a N-acetylgalactosamine (GalNAc) moiety.

Embodiment 27: The dsRNAi agent of Embodiment 25 or 26, wherein the ligand has a structure of:

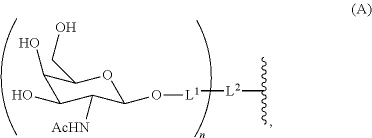

wherein:
each L¹ is independently a linker which may be same or different in each occurrence;
L² is a linker;
n is an integer from 1 to 3; or,

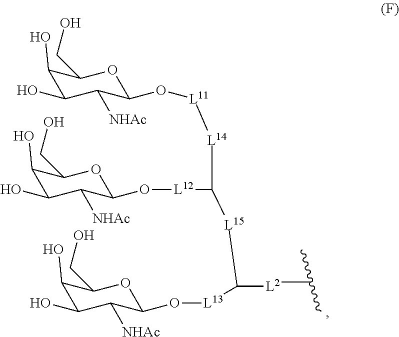

wherein:
each L¹¹, L¹², L¹³, L¹⁴, and L¹⁵ is an independently a linker;
L² is a linker;
wherein [symbol] is an attachment point to the sense strand.

Embodiment 28: The dsRNAi agent of Embodiment 27, wherein the ligand comprises the following structure of

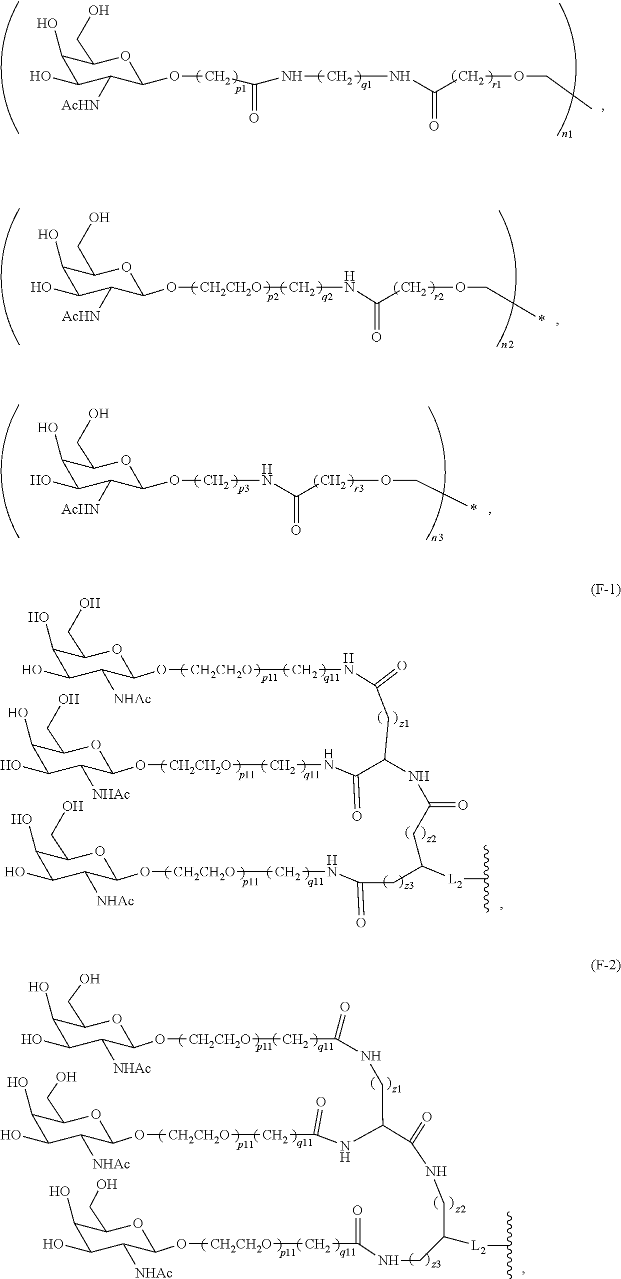

wherein:
each p1, p2, p3, p11, q1, q2, q11, r1, r2, r3, z1, z2, and z3 is independently an integer from 0 to 12;
each n1, n2, and n3 is independently an integer from 1 to 3; and “*” is an attachment point to L².

Embodiment 29: The dsRNAi agent of any one of Embodiments 25 through 28, wherein the ligand comprises the following structure:

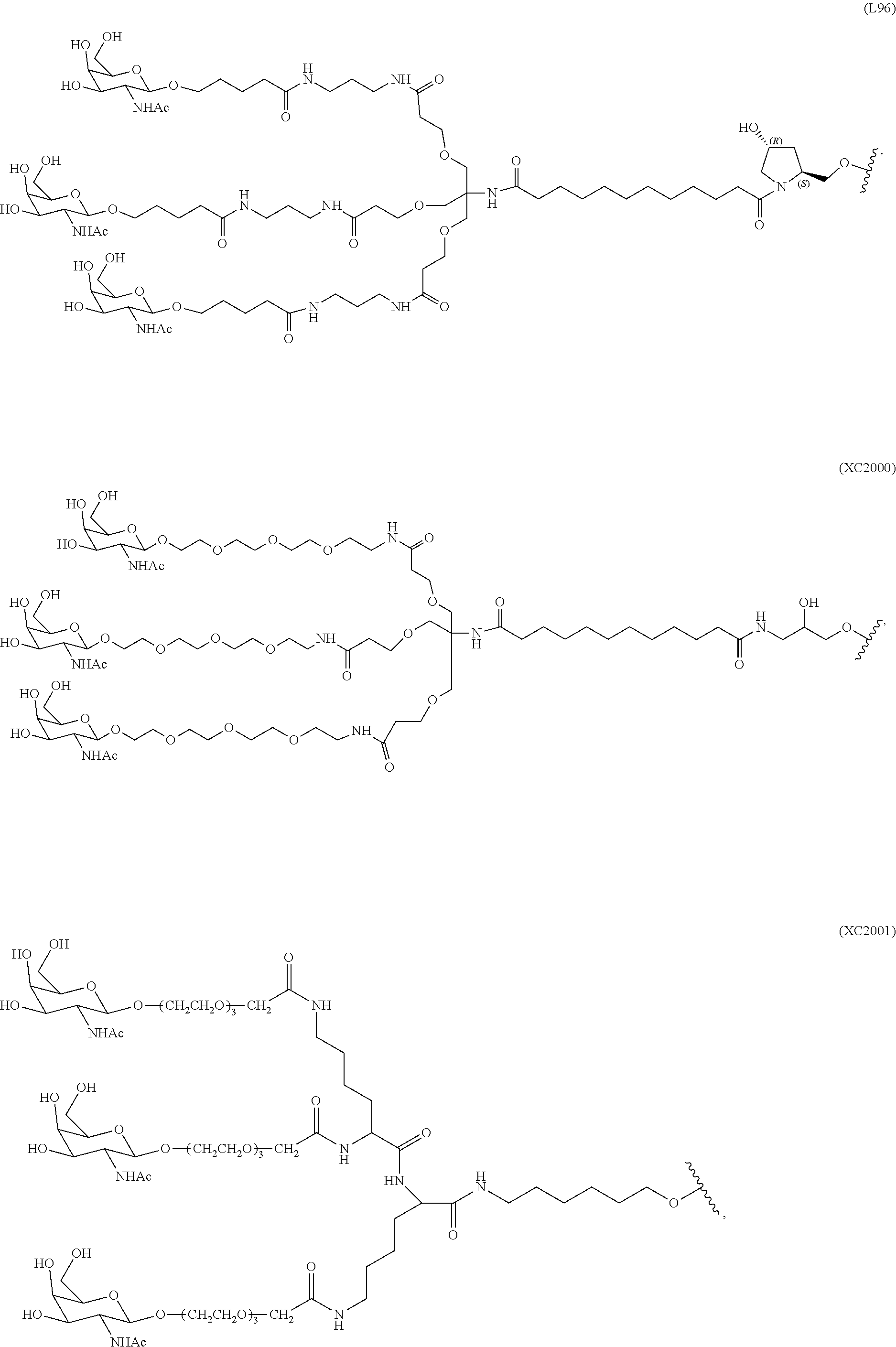

wherein
[symbol] is an attachment point to the sense strand.

Embodiment 30: The dsRNAi agent of Embodiment 29, wherein the ligand is conjugated to the 3′-end of the nucleotide sequence of the sense strand to form the following structure:

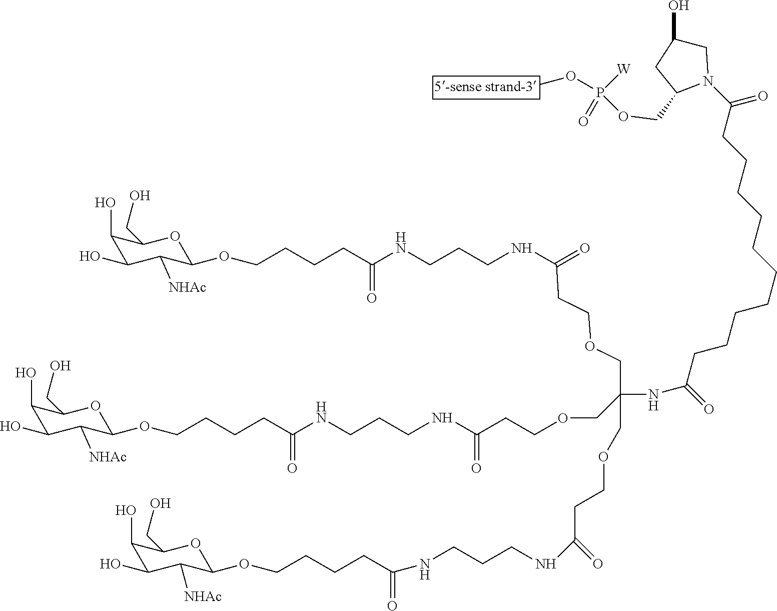

wherein W is —OH or —SH.

Embodiment 31: The dsRNAi agent of Embodiment 30, wherein W is —OH.

Embodiment 32: The dsRNAi agent of any one of Embodiments 1 through 31, wherein the dsRNAi agent is in a pharmaceutically acceptable salt form.

Embodiment 33: The dsRNAi agent of Embodiment 32, wherein the pharmaceutically acceptable salt is a sodium salt.

Embodiment 34: A double stranded RNAi (dsRNAi) agent comprising:
(i) a sense strand comprising a nucleotide sequence of SEQ ID NO: 1302, and an antisense strand comprising a nucleotide sequence of SEQ ID NO: 1306;
(ii) a sense strand comprising a nucleotide sequence of SEQ ID NO: 1303, and an antisense strand comprising a nucleotide sequence of SEQ ID NO: 1307;
(iii) a sense strand comprising a nucleotide sequence of SEQ ID NO: 1304, and an antisense strand comprising a nucleotide sequence of SEQ ID NO: 1308; or
(iv) a sense strand comprising a nucleotide sequence of SEQ ID NO: 1305; and an antisense strand comprising a nucleotide sequence of SEQ ID NO: 1309,
wherein the ligand is conjugated to the 3′-end of the nucleotide sequence of the sense strand to form the following structure:

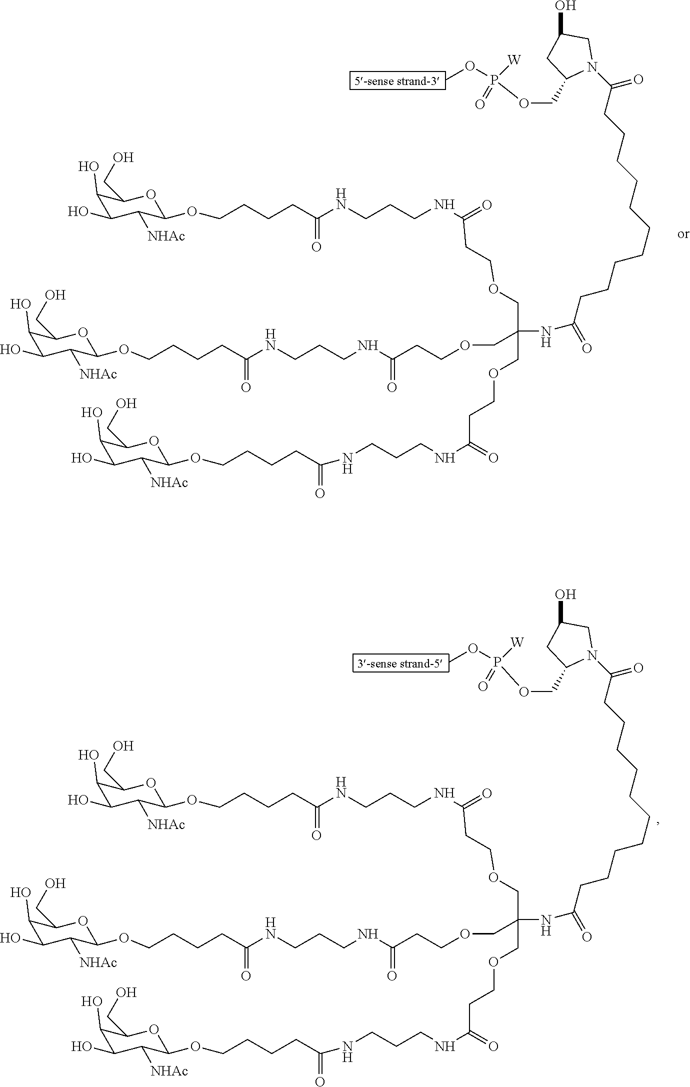

of a pharmaceutically acceptable salt,
wherein W is —OH.

Embodiment 35: The dsRNAi agent of Embodiment 34, wherein the dsRNAi agent is in a pharmaceutically acceptable salt form.

Embodiment 36: The dsRNAi agent of Embodiment 35, wherein the pharmaceutically acceptable salt is a sodium salt.

Embodiment 37: A double stranded RNAi (dsRNAi) agent comprising:
(i) a sense strand consisting of a nucleotide sequence of SEQ ID NO: 1302, and an antisense strand consisting of a nucleotide sequence of SEQ ID NO: 1306;
(ii) a sense strand consisting of a nucleotide sequence of SEQ ID NO: 1303, and an antisense strand consisting of a nucleotide sequence of SEQ ID NO: 1307;
(iii) a sense strand consisting of a nucleotide sequence of SEQ ID NO: 1304, and an antisense strand consisting of a nucleotide sequence of SEQ ID NO: 1308; or
(iv) a sense strand consisting of a nucleotide sequence of SEQ ID NO: 1305; and an antisense strand consisting of a nucleotide sequence of SEQ ID NO: 1309,
wherein the ligand (L96) is conjugated to the 3′-end of the nucleotide sequence of the sense strand to form the following schematic:

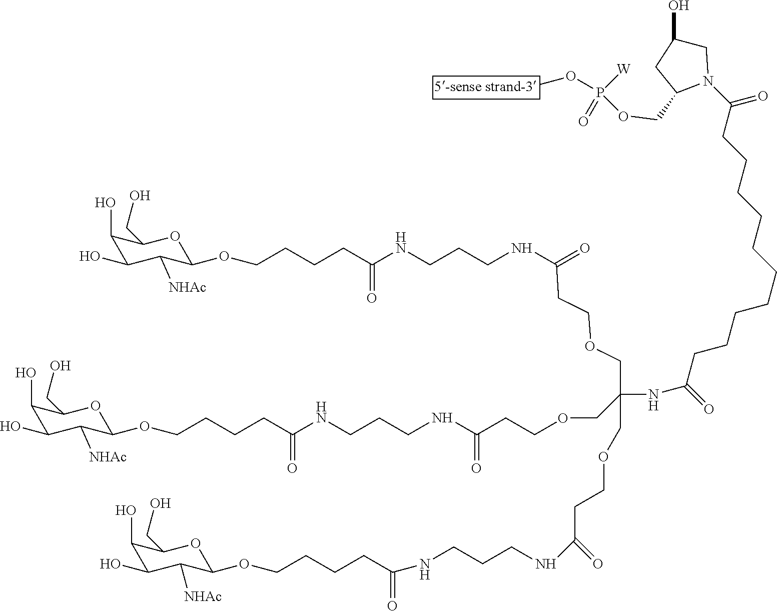

or
wherein the ligand (L96) is conjugated to the 5′-end of the sense strand to form the following schematic:

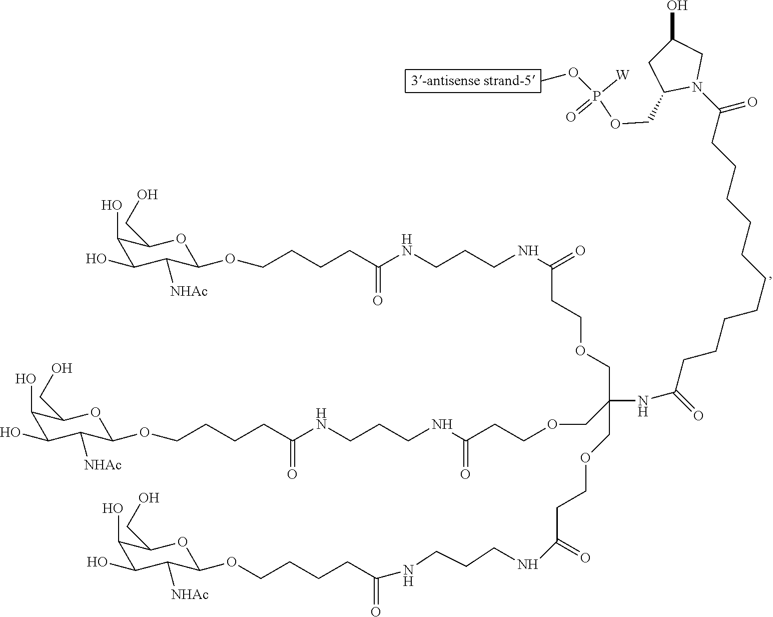

or a pharmaceutically acceptable salt,
   wherein W is —OH.

Embodiment 38: The dsRNAi agent of Embodiment 37, wherein the dsRNAi agent is in a pharmaceutically acceptable salt form.

Embodiment 39: The dsRNAi agent of Embodiment 38, wherein the pharmaceutically acceptable salt is a sodium salt.

Embodiment 40: A pharmaceutical composition comprising the dsRNAi agent of any one of Embodiments 1 through 39, and a pharmaceutically acceptable carrier.

Embodiment 41: The pharmaceutical composition of Embodiment 40, wherein the composition is in an aqueous solution form.

Embodiment 42: The pharmaceutical composition of Embodiment 40 through 41, further comprising an additional therapeutic agent selected from a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

Embodiment 43: The pharmaceutical composition of Embodiment 42, wherein the additional therapeutic agent comprises the PCSK9 inhibitor.

Embodiment 44: The pharmaceutical composition of Embodiment 43, wherein the PCSK9 inhibitor is a second dsRNAi agent.

Embodiment 45: The pharmaceutical composition of Embodiment 44, wherein the second dsRNAi agent comprises inclisiran.

Embodiment 46: A combination of the dsRNAi agent of any one of Embodiments 1 through 39 and a second agent selected from a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

Embodiment 47: The combination of Embodiment 46, wherein the second agent is a second dsRNAi agent.

Embodiment 48: The combination of Embodiment 47, wherein the second dsRNAi agent is a dsRNAi agent that targets one or more of the genes selected from the group consisting of PCSK9, LPA, AGT, ACE, ACE2, AGTR1, AGTR2, ACAT, CETP, MTTP, PPAR, IBAT, FDFT1, ERG9, SQS1, Ccl2, CCR2, CCL7, CCL8, CCL13, and CCL16.

Embodiment 49: The combination of Embodiment 47 or 48, wherein the second dsRNAi agent comprises inclisiran.

Embodiment 50: A pharmaceutical composition comprising the combination of any one of Embodiments 46 through 49.

Embodiment 51: The pharmaceutical composition of Embodiment 50, wherein the second dsRNAi agent is in a pharmaceutically acceptable salt form.

Embodiment 52: The pharmaceutical composition of Embodiment 51, wherein the pharmaceutically acceptable salt of the second dsRNAi agent is a sodium salt.

Embodiment 53: The pharmaceutical composition of any one of Embodiments 50 to 52, wherein the dsRNAi agent and the second agent are formulated in the same composition.

Embodiment 54: The pharmaceutical composition of any one of Embodiments 50 to 52, wherein the dsRNAi agent and the second agent are formulated in the separate compositions.

Embodiment 55: A method of inhibiting expression of 3-hydroxy-3-methylglutaryl-CoA reductase (HMGCR) in a subject comprising:
administering to the subject the dsRNAi agent of any one of Embodiments 1 through 39 or a pharmaceutically acceptable salt thereof, or the pharmaceutical composition of any one of Embodiments 40 through 45.

Embodiment 56: A method of lowering a level of low-density lipoprotein cholesterol (LDL-C) in a subject, comprising:
administering to the subject the dsRNAi agent of any one of Embodiments 1 through 39 or a pharmaceutically acceptable salt thereof, or the pharmaceutical composition of any one of Embodiments 40 through 45.

Embodiment 57: A method of treating or preventing an HMGCR-associated disorder or disease in a subject, comprising:
administering to the subject the dsRNAi agent of any one of Embodiments 1 through 39 or a pharmaceutically acceptable salt thereof, or the pharmaceutical composition of any one of Embodiments 40 through 45.

Embodiment 58: The method of Embodiment 57, wherein the HMGCR-associated disorder or disease is hyperlipidemia, hypercholesterolemia, hypertriglyceridemia, mixed hyperlipidemia, congestive heart disease (CHD) or atherosclerosis.

Embodiment 59: A method of treating or preventing hyperlipidemia in a subject, comprising:
administering to the subject the dsRNAi agent of any one of Embodiments 1 through 39 or a pharmaceutically acceptable salt thereof, or the pharmaceutical composition of any one of Embodiments 40 through 45.

Embodiment 60: The method of Embodiment 59, wherein the hyperlipidemia is hypercholesterolemia, or hypertriglyceridemia.

Embodiment 61: A method of treating or preventing atherosclerotic cardiovascular disease (ASCVD) in a subject, comprising:
administering to the subject the dsRNAi agent of any one of Embodiments 1 through 39 or a pharmaceutically acceptable salt thereof, or the pharmaceutical composition of any one of Embodiments 40 through 45.

Embodiment 62: The method of any one of Embodiments 55 through 61, wherein the dsRNAi agent or the pharmaceutical composition is administered subcutaneously or intravenously.

Embodiment 63: The method of any one of Embodiments 55 through 62, further comprising administering to the subject an additional therapeutic agent selected from a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

Embodiment 64: The method of Embodiment 63, wherein the additional therapeutic agent is a second dsRNAi agent.

Embodiment 65: The method of Embodiment 64, wherein the second dsRNAi agent comprises a PCSK9 inhibitor.

Embodiment 66: The method of Embodiment 65, wherein the second dsRNAi agent comprises inclisiran.

Embodiment 67: The method of any one of Embodiments 63 through 66, wherein the dsRNAi agent or the pharmaceutical composition and the additional therapeutic agent are administered simultaneously.

Embodiment 68: The method of any one of Embodiments 55 through 67, wherein the dsRNAi agent or the pharmaceutical composition and the additional therapeutic agent are administered subsequently.

Embodiment 69: The method of Embodiment 68, wherein the dsRNAi agent is administered before administering the additional therapeutic agent.

Embodiment 70: The method of Embodiment 68, wherein the additional therapeutic agent is administered before administering the dsRNAi agent.

Embodiment 71: The method of any one of Embodiments 63 through 70, wherein the additional therapeutic agent is administered subcutaneously or intravenously.

Embodiment 72: The method of any one of Embodiments 55 through 71, wherein the subject is a human.

Embodiment 73: The method of any one of Embodiments 55 through 72, wherein the subject has or is diagnosed with hyperlipidemia, hypercholesterolemia, hypertriglyceridemia, mixed hyperlipidemia, congestive heart disease (CHD) or atherosclerosis.

Embodiment 74: A method of lowering a level of low-density lipoprotein cholesterol (LDL-C) in a subject, comprising:
administering to the subject the pharmaceutical composition of any one of Embodiments 50 through 54.

Embodiment 75: A method of treating or preventing an HMGCR-associated disorder or disease in a subject, comprising:
administering to the subject the pharmaceutical composition of any one of Embodiments 50 through 54.

Embodiment 76: The method of Embodiment 74, wherein the HMGCR-associated disorder or disease is hyperlipidemia, hypercholesterolemia, hypertriglyceridemia, mixed hyperlipidemia, congestive heart disease (CHD) or atherosclerosis.

Embodiment 77: A method of treating or preventing hyperlipidemia in a subject, comprising:
administering to the subject the pharmaceutical composition of any one of Embodiments 50 through 54.

Embodiment 78: A method of treating or preventing atherosclerotic cardiovascular disease (ASCVD) in a subject, comprising:
administering to the subject the pharmaceutical composition of any one of Embodiments 50 through 54.

Embodiment 79: The method of any one of Embodiments 74 through 78, wherein the dsRNAi agent and the second agent is administered subcutaneously or intravenously.

Embodiment 80: The method of any one of Embodiments 74 through 79, wherein the dsRNAi agent and the second agent are administered simultaneously.

Embodiment 81: The method of any one of Embodiments 74 through 79, wherein the dsRNAi agent and the second agent are administered subsequently.

Embodiment 82: The method of Embodiment 81, wherein the dsRNAi agent is administered before administering the second agent.

Embodiment 83: The method of Embodiment 81, wherein the second agent is administered before administering the dsRNAi agent.

Embodiment 84: The method of any one of Embodiments 74 through 83, wherein the subject is a human.

Embodiment 85: The method of any one of Embodiments 74 through 84, wherein the subject has or is diagnosed with hyperlipidemia, hypercholesterolemia, hypertriglyceridemia, mixed hyperlipidemia, congestive heart disease (CHD) or atherosclerosis.

Embodiment 86: A kit comprising the dsRNAi agent of any one of Embodiments 1 through 39 or a pharmaceutically acceptable salt thereof, or the pharmaceutical composition of any one of Embodiments 40 through 45.

Embodiment 87: The kit of Embodiment 86, further comprising an additional therapeutic agent selected from a proprotein convertase subtilisin kexin 9 (PCSK9) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

Embodiment 88: The kit of Embodiment 87, wherein the additional therapeutic agent is a second dsRNAi agent.

Embodiment 89: The kit of Embodiment 88, wherein the second dsRNAi agent is a dsRNAi agent that targets one or more of the genes selected from the group consisting of PCSK9, LPA, AGT, ACE, ACE2, AGTR1, AGTR2, ACAT, CETP, MTTP, PPAR, IBAT, FDFT1, ERG9, SQS1, Ccl2, CCR2, CCL7, CCL8, CCL13, and CCL16.

Embodiment 90: The kit of Embodiment 89, wherein the second dsRNAi agent comprises a PCSK9 inhibitor.

Embodiment 91: The kit of Embodiment 90, wherein the second dsRNAi agent comprises inclisiran.

Embodiment 92: The kit of any one of Embodiments 87 through 91, wherein the dsRNAi agent and the additional therapeutic agent are contained in a single vial.

Embodiment 93: The kit of any one of Embodiments 87 through 91, wherein the dsRNAi agent and the additional therapeutic agent are contained in separate vials.

Embodiment 94: The kit of any one of Embodiments 86 through 93, further comprising one or more applicators.

Embodiment 95: The kit of Embodiment 94, wherein the one or more applicators includes a syringe.

Embodiment 96: A kit comprising the pharmaceutical composition of any one of Embodiments 50 through 54.

Embodiment 97: The kit of Embodiment 96, wherein the dsRNAi agent and the second agent are contained in a single vial.

Embodiment 98: The kit of Embodiment 96, wherein the dsRNAi agent and the second agent are contained in separate vials.

Embodiment 99: The kit of any one of Embodiments 96 through 98, further comprising one or more applicators.

Embodiment 100: The kit of Embodiment 99, wherein the one or more applicators are syringes.

### Embodiments (II)

Embodiment P1: A double stranded RNAi (dsRNAi) agent comprising:
a sense strand comprising a nucleotide sequence selected from (i) SEQ ID NOs: 1 to 405 in Table 1, or (ii) SEQ ID NOs: 1 to 405 and 1434 to 1440 in Table 1; and
an antisense strand forming a duplex with the sense strand and comprising a nucleotide sequence selected from (i) SEQ ID NOs: 406 to 810 in Table 1, or (ii) SEQ ID NOs: 406 to 810 and 1441 to 1447 in Table 1.

Embodiment P2: The dsRNAi agent of Embodiment P1, wherein the sense strand is 21 to 23 nucleotides in length and the antisense strand is 23 to 25 nucleotides in length.

Embodiment P3: The dsRNAi agent of Embodiment P1 or P2, wherein all the nucleotides in the sense strand and the antisense strand are modified nucleotides.

Embodiment P4: The dsRNAi agent of Embodiments P1 through P3, wherein each of the modified nucleotides independently comprises one or more modifications selected from a 2′-deoxy modification, a 2′-O-alkyl modification, a 2′-halo modification, a 2′-5′-linkage modification, a conformationally restricting modification, an abasic modification, a 2′-amino-modification, a 2′-O-allyl modification, 2′-C-alkyl modification, a 2′-O-alkoxyalkyl modification, a morpholino modification, a modification containing a phosphoramidate group, a modification containing a non-natural nucleobase, a modification in a tetrahydropyran, a modification containing a threose nucleic acid (TNA), a modification containing a 1,5-anhydrohexitol, a modification containing a cyclohexyl, a modification containing a cyclohexenyl, a modification containing a phosphorothioate group, a modification containing a methylphosphonate group, a modification containing an alkylphosphate, a modification containing a phosphonate, a modification containing an alkylphosphonate, a modification to form a thermally destabilizing nucleotide, a modification containing a glycol nucleic acid (GNA), and a 2-O-(N-methylacetamide) modification.

Embodiment P5: The dsRNAi agent of Embodiment P4, wherein each of the modified nucleotides is independently selected from GNA, 2′O-alkoxyalkyl modified nucleotide, 2′-O-alkyl modified nucleotide, 2′-O-allyl modified nucleotide, 2′-C-alkyl modified nucleotide, and 2′-halo modified nucleotide, and optionally comprises one or more modifications selected from a modification containing a phosphorothioate group, a modification containing a methylphosphonate group, a modification containing an alkylphosphate, a modification containing a phosphonate, a modification containing an alkylphosphonate, and an abasic modification.

Embodiment P6: The dsRNAi agent of any one of Embodiments P3 through P5, wherein all the modified nucleotides comprise a modification on a 2′ sugar ring.

Embodiment P7: The dsRNAi agent of Embodiment P6, wherein the modified nucleotides are selected from a 2′-O-alkyl modified nucleotide, a 2′-halo modified nucleotide, a 2′-deoxy modified nucleotide, and a 2′-O-alkoxyalkyl modified nucleotide.

Embodiment P8: The dsRNAi agent of Embodiment P6 or P7, wherein one or more of the modified nucleotides further comprises a 3′-phosphorothioate (PS) modification.

Embodiment P9: The dsRNAi agent of any one of Embodiments P4 through P8, wherein each of the modified nucleotides comprises one or more modifications selected from 2′-O-methyl (2′-OMe) modification, 2′-fluoro (2′-F) modification, 2′-O-methoxyethyl (2′-MOE) modification, 3′-phosphorothioate (PS) modification, and 5′-vinyl-phosphonate (5′-VP) modification.

Embodiment P10: The dsRNAi agent of any one of Embodiments P1 through P9, wherein the sense strand comprises one or two 2′-MOE modified nucleotides positioned at the 1^{st} and/or 2^{nd} nucleotides from the 5′-end of the sense strand.

Embodiment P11: The dsRNAi agent of any one of Embodiments P1 to P10, wherein the sense strand comprises one or two 2′-MOE modified nucleotides positioned at the 1^{st} and/or 2^{nd} nucleotides from the 3′-end of the sense strand.

Embodiment P12: The dsRNAi agent of any one of Embodiments P1 through P11, wherein the antisense strand comprises a 5′-VP group at the 1^{st} nucleotide from 5′ end of the antisense strand.

Embodiment P13: The dsRNAi agent of any one of Embodiments P1 through P11, wherein the antisense strand comprises a 5′-(E)-VP group at the 1^{st} nucleotide from 5′ end of the antisense strand.

Embodiment P14: The dsRNAi agent of any one of Embodiments P1 through P11, wherein the antisense strand comprises a 5′-(E)-VP-2′-OMe nucleotide at the 1^{st} position from 5′ end of the antisense strand.

Embodiment P15: The dsRNAi agent of any one of Embodiments P1 and P14, wherein each of the sense strand and the antisense strand independently comprises two, three, four, five or six 2′-F modified nucleotides.

Embodiment P16: The dsRNAi agent of any one of Embodiments P1 through P15, wherein the sense strand comprises one or two 3′-PS group at the 1^{st} and/or 2^{nd} nucleotides from 5′-end of the sense strand.

Embodiment P17: The dsRNAi agent of any one of Embodiments P1 through P16, wherein the antisense strand comprises one or two 3′-PS group at the 1^{st} and/or 2^{nd} nucleotides from 5′-end of the antisense strand, and/or one or two 3′-PS group at the 1^{st} and/or 2^{nd} nucleotides from 3′-end of the antisense strand.

Embodiment P18: The dsRNAi agent of any one of Embodiments P1 to P17, wherein the sense strand is 21 nucleotides in length and the antisense strand is 23 nucleotides in length.

Embodiment P19: The dsRNAi agent of Embodiment P18, wherein the sense strand comprises one to four 2′-MOE modified nucleotides positioned at the 1^{st}, 2^{nd}, 20^{th}, and/or 21^{st} nucleotides from the 5′-end of the sense strand.

Embodiment P20: The dsRNAi agent of Embodiment P19, wherein the sense strand comprises only four 2′-MOE modified nucleotides.

Embodiment P21: The dsRNAi agent of any one of Embodiments P18 through P20, wherein the sense strand does not comprise a 2′-MOE modified nucleotide at the 3^{rd} to 19^{th} positions from 5′-end of the sense strand.

Embodiment P22: The dsRNAi agent of Embodiment P21, wherein the sense strand comprises two, three, or four 2′-F modified nucleotides positioned at the 7^{th}, 9^{th}, 10^{th}, and/or 11^{th} nucleotide from 5′-end of the sense strand.

Embodiment P23: The dsRNAi agent of Embodiment P21, wherein the sense strand comprises 2′-F modified nucleotides positioned at the 7^{th}, 9^{th}, 10^{th}, and 11^{th} nucleotides from 5′-end of the sense strand.

Embodiment P24: The dsRNAi agent of Embodiment P22 or P23, wherein the remaining nucleotides in the sense strand comprise 2′-OMe modified modification.

Embodiment P25: The dsRNAi agent of any one of Embodiments P18 through P24, wherein the antisense strand comprises a 5′-(E)-VP group at the 1^{st} nucleotide from 5′ end of the antisense strand.

Embodiment P26: The dsRNAi agent of any one of Embodiments P18 through P25, wherein the antisense strand comprises two, three, or four 2′-F modified nucleotides positioned at the 2^{nd}, 6^{th}, 14^{th}, and/or 16^{th} nucleotides from 5′-end of the antisense strand.

Embodiment P27: The dsRNAi agent of Embodiment P26, wherein the antisense strand comprises 2′-F modified nucleotides positioned at the 2^{nd}, 6^{th}, 14^{th}, and 16^{th} nucleotides from 5′-end of the antisense strand.

Embodiment P28: The dsRNAi agent of any one of Embodiments P18 through P27, wherein the antisense strand comprises a GNA at the 5^{th} nucleotide from 5′-end of the antisense strand.

Embodiment P29: The dsRNAi agent of any one of Embodiments P25 through P28, wherein the remaining nucleotides in antisense strand comprise 2′-OMe modified modifications.

Embodiment P30: The dsRNAi agent of any one of Embodiments P18 through P29, wherein the sense strand comprises one to eight 3′-PS group at the 1^{st}, 2^{nd}, 3^{rd} 4^{th}, 17^{th}, 18^{th}, 19^{th} and/or 20^{th} nucleotides from 5′-end of the sense strand.

Embodiment P31: The dsRNAi agent of any one of Embodiments P18 through P30, wherein the antisense strand comprises one to eight 3′-PS group at the 1^{st}, 2^{nd}, 3^{rd}, 4^{th}, 19^{th}, 20^{th}, 21^{st} and/or 22^{nd} nucleotides from 5′-end of the antisense strand.

Embodiment P32: The dsRNAi agent of any one of Embodiments P16, P17, P30 and P31, wherein at least one of the 3′-PS groups in each sense strand and antisense strand has a stereopure Rp configuration.

Embodiment P33: The dsRNAi agent of any one of Embodiments P16, P17, P30 and P31, wherein at least one of the 3′-PS groups in each sense strand and antisense strand has a stereopure Sp configuration.

Embodiment P34: A double stranded RNAi (dsRNAi) agent comprising:
a sense strand having a nucleotide sequence selected from (i)SEQ ID NOs: 812 to 1052 in Table 2, or (ii) SEQ ID NOs: 812 to 1052 in Table 2 and SEQ ID NOs: 1294 to 1297 and 1448 to 1462 in Table 3;
an antisense strand forming a duplex with the sense strand and having a nucleotide sequence selected from (i) SEQ ID NOs: 1053 to 1293 in Table 2, or (ii) SEQ ID NOs: 1053 to 1293 in Table 2.

Embodiment P35: The dsRNAi agent of any one of Embodiments P1 through P34, further comprising a ligand.

Embodiment P36: The dsRNAi agent of Embodiment P35, wherein the ligand comprises a N-acetylgalactosamine (GalNAc) moiety.

Embodiment P37: The dsRNAi agent of Embodiment P35 or P36, wherein the ligand has a structure of:

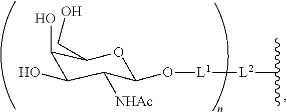

wherein:
each L¹ is independently a linker which may be same or different in each occurrence;
L² is a linker;
n is an integer from 1 to 3; and
[symbol] is an attachment point to the sense strand or an antisense strand.

Embodiment P38: The dsRNAi agent of Embodiment P37, wherein the ligand comprises the following structure of

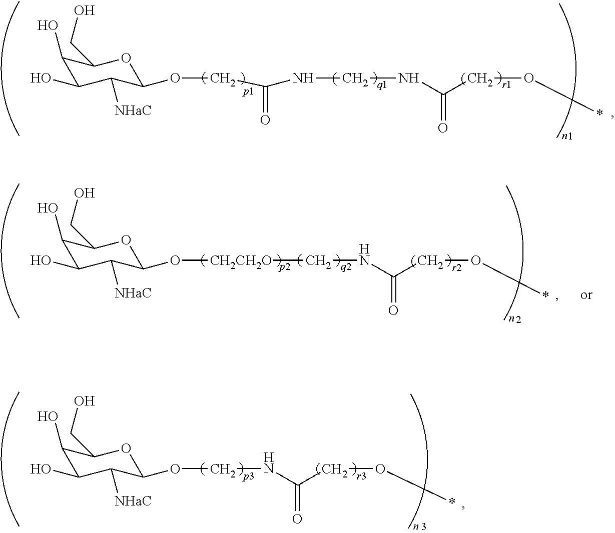

wherein:
each p1, p2, p3, q1, q2, r1, r2 and r3 is independently an integer from 0 to 12;
each n1, n2, and n3 is independently an integer from 1 to 3; and
“*” is an attachment point to L².

Embodiment P39: The dsRNAi agent of Embodiment P35 or P36, wherein the ligand has a structure of:

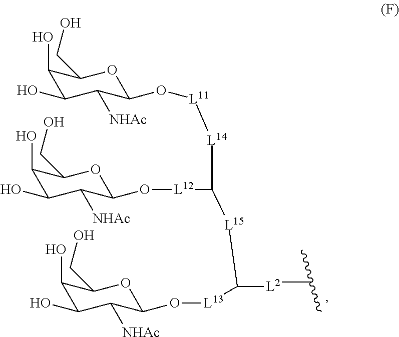

wherein:
each L¹¹, L¹², L¹³, L¹⁴, and L¹⁵ is an independently a linker;
L² is a linker;
[symbol] is an attachment point to the sense strand or the antisense strand.

Embodiment P40: The dsRNAi agent of Embodiment P39, wherein the ligand has a structure of:

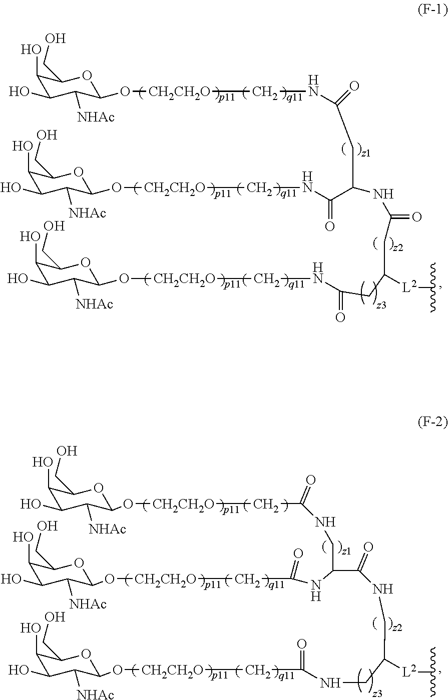

wherein:
each p11 and q11 is independently an integer from 0 to 12;
each z1, z2, and z3 is independently an integer of 0 to 12; and
[symbol] is an attachment point to the sense strand or the antisense strand.

Embodiment P41: The dsRNAi agent of any one of Embodiments P35 through P40, wherein the ligand comprises the following structure:

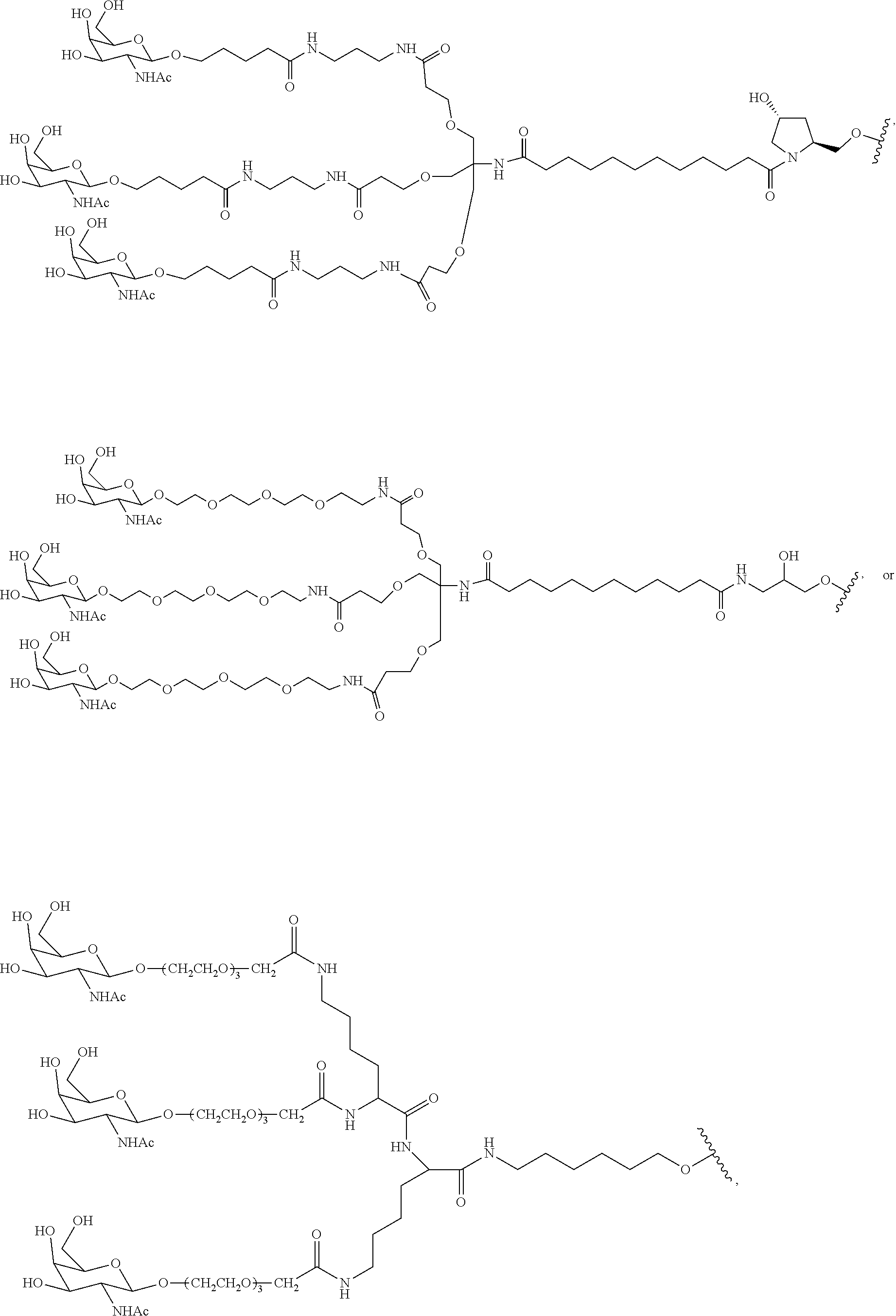

where
   [symbol] is an attachment point to the sense strand or the antisense strand.

Embodiment P42: The dsRNAi agent of Embodiment P41, wherein the ligand is conjugated to 3′ end of the sense strand to form the following structure:

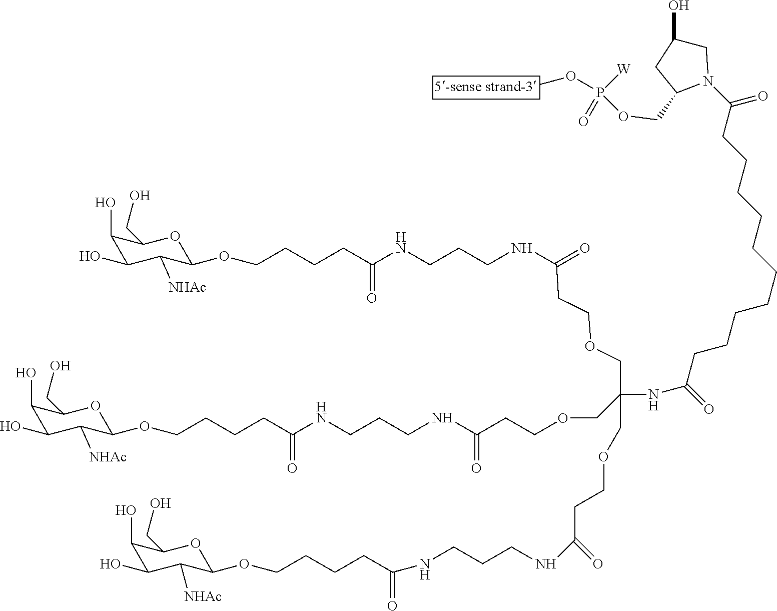

or a pharmaceutically acceptable salt,
   wherein W is —OH or —SH.

Embodiment P43: The dsRNAi agent of Embodiment P41, wherein the ligand is conjugated to 5′ end of the sense strand to form the following structure:

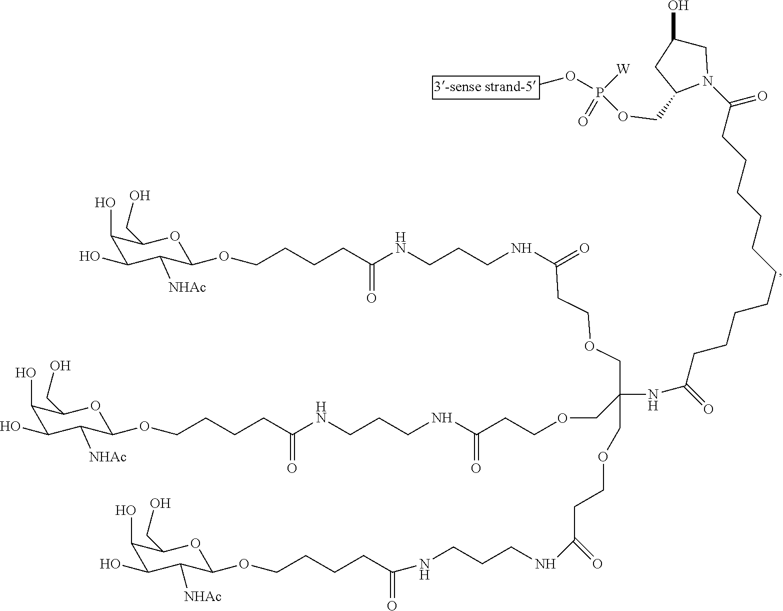

or a pharmaceutically acceptable salt,
   wherein W is —OH or —SH.

Embodiment P44: The dsRNAi agent of Embodiment P42 or P43, wherein W is —OH.

Embodiment P45: The dsRNAi agent of any one of Embodiments P1 through P44, wherein the dsRNAi agent is in a pharmaceutically acceptable salt form.

Embodiment P46: The dsRNAi agent of Embodiment P45, wherein the pharmaceutically acceptable salt is a sodium salt.

Embodiment P47: A pharmaceutical composition comprising the dsRNAi agent of any one of Embodiments P1 through P46, and a pharmaceutically acceptable carrier.

Embodiment P48: The pharmaceutical composition of Embodiment P47, wherein the composition is in an aqueous solution form.

Embodiment P49: The pharmaceutical composition of Embodiment P47 or P48, further comprising an additional therapeutic agent selected from a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

Embodiment P50: The pharmaceutical composition of Embodiment P49, wherein the additional therapeutic agent comprises the PCSK9 inhibitor.

Embodiment P51: The pharmaceutical composition of Embodiment P50, wherein the PCSK9 inhibitor is a second dsRNAi agent.

Embodiment P52: The pharmaceutical composition of Embodiment P51, wherein the second dsRNAi agent comprises inclisiran.

Embodiment P53: A combination of the dsRNAi agent of any one of Embodiments P1 through P46 and a second agent selected from a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

Embodiment P54: The combination of Embodiment P53, wherein the second agent is a second dsRNAi agent.

Embodiment P5: The combination of Embodiment P54, wherein the second dsRNAi agent is a dsRNA agent that targets one or more of the genes selected from the group consisting of PCSK9, LPA, AGT, ACE, ACE2, AGTR1, AGTR2, ACAT, CETP, MTTP, PPAR, IBA T, FDFT1, ERG9, SQS1, Ccl2, CCR2, CCL7, CCL8, CCL13, and CCL16.

Embodiment P56: The combination of any one of Embodiments P53 to P55, wherein the second dsRNAi agent comprises inclisiran.

Embodiment P57: A pharmaceutical composition comprising the combination of any one of Embodiments P53 through P56.

Embodiment P58: The pharmaceutical composition of Embodiment P57, wherein the second dsRNAi agent is in a pharmaceutically acceptable salt form.

Embodiment P59: The pharmaceutical composition of Embodiment P58, wherein the pharmaceutically acceptable salt of the second dsRNAi agent is a sodium salt.

Embodiment P60: The pharmaceutical composition of any one of Embodiments P57 to P59, wherein the dsRNAi agent and the second agent are formulated in the same composition.

Embodiment P61: The pharmaceutical composition of any one of Embodiments P57 to P59, wherein the dsRNAi agent and the second agent are formulated in the separate compositions.

Embodiment P62: A method of inhibiting expression of 3-hydroxy-3-methylglutaryl-CoA reductase (HMGCR) in a subject comprising:
administering to the subject the dsRNAi agent of any one of Embodiments P1 through P46 or a pharmaceutically acceptable salt thereof, or the pharmaceutical composition of any one of Embodiments P47 through P52.

Embodiment P63: A method of lowering a level of low-density lipoprotein cholesterol (LDL-C) in a subject, comprising:
administering to the subject the dsRNAi agent of any one of Embodiments P1 through P46 or a pharmaceutically acceptable salt thereof, or the pharmaceutical composition of any one of Embodiments P47 through P52.

Embodiment P64: A method of treating or preventing an HMGCR-associated disorder or disease in a subject, comprising:
administering to the subject the dsRNAi agent of any one of Embodiments P1 through P46 or a pharmaceutically acceptable salt thereof, or the pharmaceutical composition of any one of Embodiments P47 through P52.

Embodiment P65: The method of Embodiment P64, wherein the HMGCR-associated disorder or disease is hyperlipidemia, hypercholesterolemia, hypertriglyceridemia, mixed hyperlipidemia, congestive heart disease (CHD) or atherosclerosis.

Embodiment P66: A method of treating or preventing hyperlipidemia in a subject, comprising:
administering to the subject the dsRNAi agent of any one of Embodiments P1 through P46 or a pharmaceutically acceptable salt thereof, or the pharmaceutical composition of any one of Embodiments P47 through P52.

Embodiment P67: The method of Embodiment P66, wherein the hyperlipidemia is hypercholesterolemia, or hypertriglyceridemia.

Embodiment P68: A method of treating or preventing atherosclerotic cardiovascular disease (ASCVD) in a subject, comprising:
administering to the subject the dsRNAi agent of any one of Embodiments P1 through P46 or a pharmaceutically acceptable salt thereof, or the pharmaceutical composition of any one of Embodiments P47 through P52.

Embodiment P69: The method of any one of Embodiments P62 through P68, wherein the dsRNAi agent or the pharmaceutical composition is administered subcutaneously or intravenously.

Embodiment P70: The method of any one of Embodiments P62 through P69, further comprising administering to the subject an additional therapeutic agent selected from a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

Embodiment P71: The method of Embodiment P70, wherein the additional therapeutic agent is a second dsRNAi agent.

Embodiment P72: The method of Embodiment P71, wherein the second dsRNAi agent comprises the PCSK9 inhibitor.

Embodiment P73: The method of Embodiment P72, wherein the second dsRNAi agent comprises inclisiran.

Embodiment P74: The method of any one of Embodiments P70 through P73, wherein the dsRNAi agent or the pharmaceutical composition and the additional therapeutic agent are administered simultaneously.

Embodiment P75: The method of any one of Embodiments P70 through P73, wherein the dsRNAi agent or the pharmaceutical composition and the additional therapeutic agent are administered subsequently.

Embodiment P76: The method of Embodiment P75, wherein the dsRNAi agent is administered before administering the additional therapeutic agent.

Embodiment P77: The method of Embodiment P75, wherein the additional therapeutic agent is administered before administering the dsRNAi agent.

Embodiment P78: The method of any one of Embodiments P70 through P77, wherein the additional therapeutic agent is administered subcutaneously or intravenously.

Embodiment P79: The method of any one of Embodiments P62 through P78, wherein the subject is a human.

Embodiment P80: The method of any one of Embodiments P62 through P79, wherein the subject has or is diagnosed with hyperlipidemia, hypercholesterolemia, hypertriglyceridemia, mixed hyperlipidemia, congestive heart disease (CHD) or atherosclerosis.

Embodiment P81: The method of any one of Embodiments P62 through P80, wherein the subject does not have a muscle side effect after the administrating the dsRNAi agent of any one of Embodiments P1 through P46 or a pharmaceutically acceptable salt thereof, or the pharmaceutical composition of any one of Embodiments P47 through P52.

Embodiment P82: A method of lowering a level of low-density lipoprotein cholesterol (LDL-C) in a subject, comprising:
administering to the subject the pharmaceutical composition of any one of Embodiments P57 through P61.

Embodiment P83: A method of treating or preventing an HMGCR-associated disorder or disease in a subject, comprising:
administering to the subject the pharmaceutical composition of any one of Embodiments P57 through P61.

Embodiment P84: The method of Embodiment P83, wherein the HMGCR-associated disorder or disease is hyperlipidemia, hypercholesterolemia, hypertriglyceridemia, mixed hyperlipidemia, congestive heart disease (CHD) or atherosclerosis.

Embodiment P85: A method of treating or preventing hyperlipidemia in a subject, comprising:
administering to the subject the pharmaceutical composition of any one of Embodiments P57 through P61.

Embodiment P86: A method of treating or preventing atherosclerotic cardiovascular disease (ASCVD) in a subject, comprising:
administering to the subject the pharmaceutical composition of any one of Embodiments P57 through P61.

Embodiment P87: The method of any one of Embodiments P82 through P86, wherein the dsRNAi agent and the second agent is administered subcutaneously or intravenously.

Embodiment P88:The method of any one of Embodiments P82 through P87, wherein the dsRNAi agent and the second agent are administered simultaneously.

Embodiment P89: The method of any one of Embodiments P82 through P88, wherein the dsRNAi agent and the second agent are administered subsequently.

Embodiment P90: The method of Embodiment P89, wherein the dsRNAi agent is administered before administering the second agent.

Embodiment P91: The method of Embodiment P89, wherein the second agent is administered before administering the dsRNAi agent.

Embodiment P92: The method of any one of Embodiments P82 through P91, wherein the subject is a human.

Embodiment P93: The method of any one of Embodiments P82 through P92, wherein the subject has or is diagnosed with hyperlipidemia, hypercholesterolemia, hypertriglyceridemia, mixed hyperlipidemia, congestive heart disease (CHD) or atherosclerosis.

Embodiment P94: The method of any one of Embodiments P82 through P93, wherein the subject does not have a muscle side effect after the administrating the pharmaceutical composition of any one of Embodiments P57 through P61.

Embodiment P95: A kit comprising the dsRNAi agent of any one of Embodiments P1 through P46 or a pharmaceutically acceptable salt thereof, or the pharmaceutical composition of any one of Embodiments P47 through P52.

Embodiment P96: The kit of Embodiment P95, further comprising an additional therapeutic agent selected from a proprotein convertase subtilisin kexin 9 (PCSK9) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

Embodiment P97: The kit of Embodiment P96, wherein the additional therapeutic agent is a second dsRNAi agent.

Embodiment P98: The kit of Embodiment P97, wherein the second dsRNAi agent is a dsRNA agent that targets one or more of the genes selected from the group consisting of PCSK9, LPA, AGT, ACE, ACE2, AGTR1, AGTR2, ACAT, CETP, MTTP, PPAR, IBAT, FDFT1, ERG9, SQS1, Ccl2, CCR2, CCL7, CCL8, CCL13, and CCL16.

Embodiment P99: The kit of Embodiment P98, wherein the second dsRNAi agent comprises the PCSK9 inhibitor.

Embodiment P100: The kit of Embodiment P99, wherein the second dsRNAi agent comprises inclisiran.

Embodiment P101: The kit of any one of Embodiments P96 through P100, wherein the dsRNAi agent and the additional therapeutic agent are contained in a single vial.

Embodiment P102: The kit of any one of Embodiments P96 through P100, wherein the dsRNAi agent and the additional therapeutic agent are contained in separate vials.

Embodiment P103: The kit of any one of Embodiments P95 through P101, further comprising one or more applicators.

Embodiment P104: The kit of Embodiment P103, wherein the one or more applicators comprises a syringe.

Embodiment P105: A kit comprising the pharmaceutical composition of any one of Embodiments P57 through P61.

Embodiment P106: The kit of Embodiment P105, wherein the dsRNAi agent and the second agent are contained in a single vial.

Embodiment P107: The kit of Embodiment P105, wherein the dsRNAi agent and the second agent are contained in separate vials.

Embodiment P108: The kit of any one of Embodiments P105 through P107, further comprising one or more applicators.

Embodiment P109: The kit of Embodiment P108, wherein the one or more applicators are syringes.

### EXAMPLES

### Example 1: Bioinformatics Off-Targeting/RNA-Seq

siRNA sequences were designed to be complementary to human HMGCR isoform, transcript variant 1, mRNA (GenBank: NM_000859.3) and about four hundreds of RNAi agents (siRNAs) to HMGCR were screened and tested for off-target hybridization (e.g., less off-target hybridization) and knock-down of HMGCR mRNA in a cell. Three lead sequences, siRNA286, siRNA3, and siRNA6, were evaluated with off-target profiling.

### Material and Methods

### Cell Culture and Transfection:

Hep3B cells (ATCC HB-8064) were cultured in EMEM with L-Glut (ATCC 30-2003)+10% FBS medium (heat inactivated). Hep3B cells were plated at density of 250,000 cells/well (media volume: 1000 μL) in a 12-well plate. Cells were treated with siRNA at 20 nM using RNAiMax (Thermofisher Cat: 13778150 Lipofectamine™ RNAiMAX Transfection Reagent) in quadruplicates. As negative control, cells were seeded at same density as described above and treated with PBS with same amount of RNAiMax.

### RNA Isolation and QC:

Cells were harvested, lysed and total RNA was extracted using RNeasy 96 kit from Qiagen (Cat #74181) 24 hrs post transfection. RNA quality (RIN score >7-10) and yield (>25ng/μL) was assessed using RNA tapes (Cat #5067-5576) on Agilent 4200 TapeStation (#G2991BA). RNA was stored at −80° C. until submitted for library preparation.

### TruSeq (Illumina) Library Preparation and Sequencing:

Sequencing libraires were prepared from 150 ng-500 ng of total RNA using one of the following kits: Illumina 20020594 (mRNA TruSeq); Illumina 20040534 (mRNA Ligation); NEB E7760L (polyA mRNA workflow), following manufacturers' protocols. Library size, quality, and concentration (>2 nM) are assessed using D1000 tapes (Cat #5067-5582) on 4200 TapeStation (Agilent, Catalog No. G2991AA). Libraries were pooled equimolarly. Libraries and pools were stored at −20° C. until they were used for sequencing. Sequencing was done on the NovaSeq 6000 instrument (Illumina, Catalog No. 20012850) with dedicated reagent kits from the same manufacturer, generating paired end reads, which were trimmed to 50 bp. Our target coverage was >25 million reads per sample (library).

### Differential Gene Expression:

For alignment and gene expression quantification the Exon Quantification Pipeline (EQP) (Schuierer and Roma, 2016; version 2.5, August 2023) with STAR (Dobin et al., 2013; version 2.7.3a, August 2023) as the alignment tool to align the reads against the human genome reference files from Ensembl version 98 (Cunningham et al., 2015) was used. Differential gene expression was performed using DESeq2 (version 1.38.3) (Love et al., 2014) by comparing gene expression level of group of samples treated with siRNA against negative control (i.e. PBS with RNAiMax).

### Seed-Mediated in Silico Prediction

As most siRNA-seed binding sites are inherent to 3′UTR (Lin et al., 2005), each siRNA sense and antisense strand from position 2-7 and 2-8 were searched with brute-force against the 3′UTRome (NCBI Reference Seq Release 218, Homo sapiens). A gene was considered to be putative off-targeting if the 3′UTR of any isoform of the gene can be hit by the siRNA. The seed prediction was then matched to RNA-Seq results based on Gene Symbol. As most siRNA-seed binding sites were inherent to 3′UTR (Lin et al., 2005), each siRNA sense and antisense strand from position 2-7 and 2-8 were searched with brute-force against the 3′UTRome (NCBI RefSeq Release 218, Homo sapiens). A gene was considered to be putative off-targeting if the 3′UTR of any isoform of the gene could be hit by the siRNA. The seed prediction was then matched to RNA-Seq results based on Gene Symbol.

For each oligonucleotides (e.g., 19 mer, 20 mer, 21 mer, etc) along a target mRNA of interest, i) potency prediction score, ii) species cross reactivity and iii) specificity parameters are getting analyzed. For all three categories, an algorithm applied with specific thresholds (e.g., 0.7) for multiple parameters is used to identify functional and specific siRNAs. As a result of this every single nucleotide shift can change potency prediction score, species cross-reactivity and specificity.

For example, all siRNAs are fully complementary from position 2-23 to human reference transcript (GRCh38.p7). Internal design algorithm for specificity takes into account i) cross reactive to cyno, antisense position 2-18 is a full match to cyno gene of interest, ii) no predicted human off-targets protein coding and non-coding genes for the antisense strand position 2-18 with zero mismatches or one mismatch outside of the seed sequence (2-8), iii) no predicted human off-targets for the sense strand position 2-18 with zero mismatches, iv) no predicted cyno off-targets for the antisense strand position 2-18 with zero mismatches or one mismatch outside of the seed 2-8 no predicted cyno off-targets for the sense strand position 2-18 with zero mismatches. Furthermore, the seed region 2-8 of antisense strand does not match any known human miRNAs. Finally, siRNA does not hit any SNPs that have a MAF>0.01 in dbSNP.

In vitro transfection of an siRNA followed by transcriptome-wide studies using RNA-Seq can be combined with in silico prediction for off-targets and thereby identify seed mediated off-target effects. These off-target effects mediated by antisense strand seed (position 2-8) are found to be the main mechanism of toxicity observed in vitro and in vivo.

The workhorse cell line Hep3B that for all the initial off-target characterization, irrespective of whether on-target is expressed or not, may be used. If on-target is not expressed, then the project team needs to establish a new cell line where on-target is present. The on-target expression is important because the concentration of siRNA for running such an experiment is selected where maximum on-target knockdown is achieved with the minimal siRNA concentration. Furthermore, for interpretation the distance form on-target to nearest potential, off-target is important. Identified potential off-targets must be reproducible across multiple independent experiments and they must be regulated on a dose-dependent manner. If off-targets remain on a final selection process, additional assessment may be performed to de-risk identified off-targets by either checking whether the off-target(s) are expressed in respective target tissue, whether they are predicted by in silico, and whether they are also down regulated in tox studies. When shifting from a certain target mRNA position, e.g., sequence 126−/+5 nt (121 to 131) and thereby applying our standard filters with the in-house design algorithm, only sequences having threshold above 0.7 would be preferably selected.

### Example 2: Preparation of siRNAs

Small scale synthesis was used to prepare HMGCR siRNAs; medium and large scale syntheses can also be used to prepare these siRNAs in larger quantities.

### 2.1 Small Scale Synthesis and Purification Methods for the Initial Screens (1 Mole Scale)

Small scale synthesis was used to generate siRNAs. HMGCR sequences were synthesized on MerMade 192 synthesizer (BioAutomation, Plano, Tex.) at 1 μmol scale.

All oligonucleotides were prepared at 1 μmole scale using a MerMade 192 high-throughput synthesizer and commercially available phosphoramidite monomers, following standard protocols for solid-phase synthesis and deprotection. The GalNAc ligand was introduced at the 3′ end of the sense strand of the siRNA using a functionalized solid support, as previously described. PS linkages were prepared by oxidation of phosphite utilizing 0.1 M 3-((N,N-dimethyl-aminomethylidene)amino)-3H-1, 2, 4-dithiazole-5-thione (DDTT) in pyridine. After cleavage, deprotection, and precipitation of the products, each crude solution was desalted via size exclusion using water to elute the final oligonucleotide products. The identities and purities of all oligonucleotides were confirmed using electrospray ionization mass spectrometry (ESI-MS) and ion exchange-high-performance liquid chromatography (IEX-HPLC), respectively, and equimolar amounts of the complementary strands were annealed to provide the desired siRNA duplex. All duplexes met a purity cutoff of at least 85%.

The sequence file was converted to a text file to make it compatible for loading in the MerMade 192 synthesis software.

### 2.1.1 Synthesis, Cleavage and Deprotection:

The synthesis of HMGCR sequences can use solid supported oligonucleotide synthesis using phosphoramidite chemistry.

The synthesis of the above sequences was performed at 1 μM scale in 96 well plates. The RNA, TNA, GNA, 2′-OMe modified nucleotide, 5′-(E)-VP-2′-OMe modified nucleotide, 2′-MOE modified nucleotide, and 2′-F modified nucleotide phosphoramidite solutions were prepared at 0.1 M concentration and 5-(ethylthio)tetrazole (0.25 M Acetonitrile) was used as activator. Deblocking solution, oxidizer solution and capping solution were prepared according to standard processes.

The synthesized sequences were cleaved and deprotected in 96 well plates, using methylamine solution (a 3:1 mixture of aqueous and ethanolic solutions) in the first step and fluoride reagent in the second step. The crude sequences were precipitated using acetone:ethanol (80:20) mix and the pellet were re-suspended in 0.02M sodium acetate buffer. Samples from each sequence were analyzed by LC-MS to confirm the identity, UV for quantification and a selected set of samples by IEX chromatography to determine purity.

### 2.1.2 Purification and Desalting

HMGCR tiled sequences were purified on AKTA explorer purification system using Source 15Q column. A column temperature of 65° C. was maintained during purification. Sample injection and collection were performed in 96 well (1.8 mL-deep well) plates. A single peak corresponding to the full length sequence was collected in the eluent. The purified sequences were desalted on a Sephadex G25 column using AKTA purifier. The concentration of desalted HMGCR sequences were calculated using absorbance at 260 nm wavelength and purity was measured by ion exchange chromatography.

### 2.1.3 Annealing

Purified desalted sense and antisense single strands were mixed in equimolar amounts and annealed to form HMGCR duplexes. The duplexes were prepared at 10 uM concentration in 1×PBS buffer and tested by capillary gel electrophoresis for purity.

### 2.2 Medium Scale Synthesis and Purification (1-50 μMol)

Medium scale synthesis can also be used to generate siRNAs. Single-stranded RNAs in scales between 1 and 50 μmol were prepared by solid phase synthesis using an MerMade 12 synthesizer (BioAutomation, Plano, Tex.). Universal Support was purchased from AM Chemicals LLC (VisTa, CA) and 3′-GalNAc controlled pore glass (CPG) support (500A, loading 50-100 μmol/g) were homemade. For larger scales, empty synthesis columns (10 μmol) from Glen Research Corp. and large amidite (250 mL) and reagent bottles (2000 mL) were used. RNA and RNA containing 2′-MOE, 2′-F or 2′-O-methyl nucleotides were generated by solid phase synthesis employing the corresponding phosphoramidites (Hongene Biotech Corporation, Union City, CA). These building blocks were incorporated at selected sites within the sequence of the oligoribonucleotide chain using standard nucleoside phosphoramidite chemistry such as described in Current Protocols in Nucleic Acid Chemistry, Beaucage, S. L. et al. (Edrs.), John Wiley & Sons, Inc., New York, NY, USA. Vinylphosphonates at the 5′ end of the antisense strand were introduced by using solid-phase synthesis of 5′(E)-vinylphosphonate-2′-OMe-U phosphoramidite monomers. Phosphorothioate linkages were introduced using a solution of the 0.1 M DDTT (AM Chemicals, Oceanside, CA) in pyridine.

The synthesized HMGCR sequences were cleaved and deprotected in AMA solution (1:1 mixture of methylamine solution and 40% NH₃ aqueous solutions) for 3.5 hours or in 40% NH₃ aqueous solutions at 55° C. overnight. To deprotect 5′-Vinylphosphonates oligonucleotide, additional 3% of diethyl amine was add to deprotection solution. Preparative ion-pair reverse phase high-performance liquid chromatography (IPRP-HPLC) and IEX-HPLC were applied to purified oligonucleotide products. Samples from each sequence were analyzed by LC-MS to confirm the identity, UV absorbance at 260 nm for quantification and a selected set of samples by IPRP-HPLC and IEX-HPLC to determine purity. The identities and purities of all oligonucleotides were confirmed using electrospray ionization mass spectrometry (ESI-MS), Double stranded RNA was generated by mixing an equimolar solution of complementary strands in water or annealing buffer (typically phosphate buffered solution, PBS, Ambion, Applied Biosystems, Austin, TX) at the desired concentration. The mixture was then heated in a water bath at 85-90° C. for 5 minutes and cooled to room temperature over a period of 1-4 hours. The RNA duplex was stored at −20° C. until use.

### Example 3: Introduction of 5′-(E)-VP-2′OMe to the Antisense Strand of Example siRNAs as Development Candidates

### 3.1 Animals and Experimental Design

### 3.1.1 Maintenance Conditions

All mice were received at 10 weeks of age and acclimated for at least 3 days prior to experimentation. Animals were maintained on a 12 hour light/dark cycle at 70° F. and 50% humidity, provided water and food ad libitum.

### 3.1.2 Statement on Animal Welfare

Studies described were performed according to an institutional Animal Care and Use Committee (ACUC) approved protocol. All mice were maintained in our pathogen-free and viral-free institutional housing facilities and were sacrificed by CO2 asphyxiation, and confirmed by thoracotomy, as approved by the panel on Euthanasia at the American Veterinary Association, and in the above referenced ACUC protocol.

### 3.1.3 Study Protocol

Male, 10-week-old C57BL/6J mice (Jackson Laboratories, Bar Harbor, ME) were fed a western diet (Research Diets, 12079Bi) for 21 days prior to initiation of the study. On day 0 of the study, body weight data was collected for all animals. Mice were mechanically restrained, and 25 μL of baseline blood was collected via tail snip into EDTA-K2 treated microvette tubes (Sarstedt AG, Sarstedt, Germany) stored on ice. Blood samples were centrifuged at 16,000 g, 4° C. for 10 minutes, with resulting plasma aliquoted and frozen at −80° C. for subsequent measurement of total cholesterol levels. Each mouse was then given a single subcutaneous (SC) dose of either sterile PBS (10 mL/kg) or siRNA (6 mg/kg; 10 mL/kg) formulated in PBS. A total of n=24 mice received PBS control, n=24 mice received Compound 7 and n=12 received siRNA Compound 6. Each week, for 6 weeks, n=4 animals from each of the PBS and Compound 7 treatment groups were euthanized, while, for the Compound 6 treatment group, n=4 animals were euthanized only at 1, 3 and 6 weeks post-dose. Each week, 25 μL of blood was collected via tail snip for all mice remaining in the study, for measuring of total cholesterol in the resulting plasma. Each week, designated mice were euthanized via CO2 asphyxiation and terminal blood samples were collected via cardiac puncture using a 1 mL syringe and 25-gauge needle. After removing the needle, blood was ejected from the syringe into an EDTA-K2 treated tube (Sarstedt AG, Sarstedt, Germany) stored on ice and processed as described above with plasma being assayed for total cholesterol levels. The abdomen was then opened, the left lobe of liver excised and (4) 50-75 mg pieces of liver tissue were placed into individual 2 mL Eppendorf tubes, before being frozen on dry ice and stored at −80° C. until analysis.

### 3.2 Methods

3.2.1 Determination of Liver HMGCR mRNA Abundance by RT-PCR

Liver RNA extraction was performed using RNeasy lipid mini kit protocol (Qiagen, Germany). Briefly, a 50 mg piece of frozen liver was lysed and homogenized using a Tissue Lyser II (Qiagen, Germany) with one stainless steel bead and Qiazol lysis reagent (Qiagen, Germany). Next, chloroform was added, and phases were separated. RNA was bound, washed, and eluted from RNEasy mini spin columns. The optional on column DNase digestion was performed using the RNase free DNase set (Qiagen, Germany). A total of 40 μL of RNA was eluted from each sample. RNA samples were quantified using NanoDrop 2000 (ThermoFisher, Waltham MA). Quantified RNA samples were diluted and then reverse transcribed using SuperScript Vilo Master mix (ThermoFisher, Waltham MA). Taqman qPCR was performed with the cDNA samples using Taqman gene expression assays Rn00565598_m1 and Rn01455646_m1 (ThermoFisher, Waltham MA).

3.2.2 Incorporation of Guide Strand into Liver RNA-Silencing Complex

### Tissue Lysis:

Each frozen tissue piece provided in screw cap cryo tube was transferred to a 2 mL round bottom microcentrifuge tube that was pre-chilled on dry ice. One dry ice pre-chilled 5 mm stainless steel bead (Qiagen, Germany) was added to the tube containing the frozen tissue. In the cold room, sample tubes were quickly removed from dry ice and added to each tube 1 mL of ice-cold Lysis buffer (50 mM Tris-HCl pH 7.5, 150 mM NaCl, 2 mM EDTA, 0.5% Triton X-100, 1 mM PMSF, 1×EDTA-free protease inhibitor cocktail). Immediately, tissue was lysed with TissueLyser LT (Qiagen, Germany) for 5 min at 50 Hz in cold room. Lysate was then cleared at 20000×g, 10 min, 4° C., and the soluble lysate supernatants were kept on ice. The protein concentration of the soluble lysate for each sample was determined using BCA assay (ThermoFisher, Waltham MA) according to manufacturer's protocol.

### Argonaute 2 Immunoprecipitation (IP):

Dynabead Protein G (ThermoFisher, Waltham MA) was washed with Wash buffer (50 mM Tris-HCl pH 7.5, 150 mM NaCl, 2 mM EDTA, 0.5% Triton X-100) prior to use for IP, and 50 μL of bead slurry was used per sample. Mouse argonaute2 (Ago2) antibody (FUJIFILM Wako Chemicals, Richmond VA) was pre-bound to beads in wash buffer at 4° C., for 2 h on rotating mixer (200 ng antibody used per 50 μL bead slurry). After incubation, the Ago2 antibody-bound beads were washed with Wash buffer, and 50 μL of the suspension was distributed to a 1.5 mL microcentrifuge tube per sample. For each sample, Ago2 antibody-bound beads were incubated with 500 μg soluble tissue lysate in lysis buffer at final volume of 250 μL per sample at 4° C., overnight on rotating mixer. After incubation, beads in each tube were washed 5 times with 1 mL ice cold Wash buffer. Final resuspension of beads was with 50 μL PBST (phosphate buffered saline pH 7.4, 0.25% Triton X-100) per sample. siRNAs were released from bead by heating at 95° C., 5 min. Ago2 IP eluate supernatants were recovered and kept on ice on the same day or stored at −80° C. until the subsequent stem loop-reverse transcription quantitative polymerase chain reaction (SL-RT-qPCR) step.

siRNA Standard Preparation:

siRNA was first diluted to a working stock of 10 ng/μL in H2O, then further diluted 100-fold to 100 ng/mL in PBST. siRNA standards were prepared by 10-fold serial dilution from 100 ng/mL to 0.00001 ng/mL in PBST.

### Stem Loop-Reverse Transcription Quantitative Polymerase Chain Reaction (SL-RT-qPCR):

For SL-RT-qPCR, Custom Small RNA Assay (4398987, ThermoFisher, Waltham MA) containing a set of SL-RT primer and Taqman qPCR primer against guide strand sequence was designed and ordered and cDNA was generated following manufacturer's protocol of the Taqman MicroRNA Reverse Transcription Kit (ThermoFisher, Waltham MA), using 5 μL of Ago2 TP eluate or 5 μL siRNA standard. The cDNA generated (15 μL reaction) were then diluted with 75 μL H2O prior to usage for qPCR step. qPCR was performed following manufacturer's protocol for TaqMan™ Fast Advanced Master Mix (ThermoFisher, Waltham MA), using 4 μL of the diluted cDNA.

### Calculations:

An siRNA standard curve was generated by plotting Ct values (Y) versus siRNA concentration (X) in log scale using GraphPad Prism (version 9.4.1), followed by semi-log line fitting to determine slope and y-intercept values. siRNA concentration for each sample was calculated using the obtained Ct value and the determined slope and y-intercept values.

$$ng siRNA = [ siRNA ] \times Ago 2 IP elution volume ( 50 μ L )$$

$$ng siRNA per g soluble lysate protein = ng siRNA per 500 μ g ( amount used in Ago 2 IP )$$

### Data Analysis:

Statistical significance was determined by ordinary one-way ANOVA and Dunnett's multiple comparisons test using GraphPad Prism software (version 9.4.1).

### Example 4: Introduction of 2′MOE Clamps on Sense Strand of siRNA

### 4.1 Animals and Experimental Design

### Maintenance Conditions:

All mice were received at 10 weeks of age and acclimated for at least 3 days prior to experimentation. Animals were maintained on a 12 hour light/dark cycle at 70° F. and 50% humidity, provided water and food ad libitum.

Statement on Animal Welfare: Studies described were performed according to an institutional Animal Care and Use Committee (ACUC) approved protocol. All mice were maintained in our approved pathogen-free and viral-free institutional housing facilities and were sacrificed by CO2 asphyxiation, and confirmed by thoracotomy, as approved by the panel on Euthanasia at the American Veterinary Association, and in the above referenced ACUC protocol.

TABLE 7
Treatment Groups

| Treatment (siRNA) | Animals (n) |
|---|---|
| Compound 5 | 4 |
| Compound 7 | 5 |
| Compound 8 | 10 |
| PBS control | 8 |

### 4.2 Study Protocol

Male C57BL/6 mice (Jackson Laboratories, Bar Harbor, ME) were fed a western diet (Research Diets, 12079Bi) for 28 days prior to initiation of the study. On day 0 of the study, body weight data was collected for all animals. Mice were mechanically restrained, and 25 μL of baseline blood was collected via tail snip into EDTA-K2 treated microvette tubes (Sarstedt AG, Sarstedt, Germany) stored on ice. Blood samples were centrifuged at 16,000 g, 4° C. for 10 minutes, with resulting plasma aliquoted and frozen at −80° C. for subsequent measurement of total cholesterol levels. Each mouse was then given a single subcutaneous (SC) dose of either sterile PBS (10 mL/kg) or siRNA (3 mg/kg; 10 mL/kg) formulated in PBS. Each week, 25 μL of blood was collected via tail snip for all mice in the study, for measuring of total cholesterol in the resulting plasma. After 5 weeks post-dose, all animals from Compound 5 and Compound 7 treated groups and half of the animals from Compound 8 and PBS treated groups were euthanized. At 8 weeks post-dose, all mice remaining in the study were euthanized. Designated mice were euthanized via CO2 asphyxiation and terminal blood samples were collected via cardiac puncture using a 1 mL syringe and 25-gauge needle. After removing the needle, blood was ejected from the syringe into an EDTA-K2 treated tube (Sarstedt AG, Sarstedt, Germany) stored on ice and processed as described above with plasma being assayed for total cholesterol levels. The abdomen was then opened, the left lobe of liver excised and (4) 50-75 mg pieces of liver tissue were placed into individual 2 mL Eppendorf tubes, before being frozen on dry ice and stored at −80° C. until analysis.

### 4.3 Methods

All methods are identical to those for the above experiment.

### Example 5: No Significant Findings with Rodent Cross-Reactive siRNA in Rat DRF Study

### 5.1 Animals and Experimental Design

On study day 1, 9- to 10-week old male Wistar Han rats received a single subcutaneous injection (dorsal mid-scapular region) of vehicle (0.9% sodium chloride for injection, USP) or HMGCR GalNAc-conjugated siRNA at a dose of 10, 30, 100 or 300 mg/kg (n=5 rats per group). At necropsy (30 days post-dose), liver and right bicep femoris skeletal muscle samples for the measurement of HMGCR mRNA abundance were collected from all animals.

### 5.2 Methods

5.2.1 Determination of Liver HMGCR mRNA Abundance

Liver RNA extraction was performed using RNeasy lipid mini kit protocol (Qiagen, Germany). Briefly, a 50 mg piece of frozen liver was lysed and homogenized using a Tissue Lyser II (Qiagen, Germany) with one stainless steel bead and Qiazol lysis reagent (Qiagen, Germany). Next, chloroform was added, and phases were separated. RNA was bound, washed, and eluted from RNEasy mini spin columns. The optional on column DNase digestion was performed using the RNase free DNase set (Qiagen, Germany). A total of 40 μL of RNA was eluted from each sample. RNA samples were quantified using NanoDrop 2000 (ThermoFisher, Waltham MA). Quantified RNA samples were diluted and then reverse transcribed using SuperScript Vilo Master mix (ThermoFisher, Waltham MA). Taqman qPCR was performed with the cDNA samples using Taqman gene expression assays Rn00565598_m1 and Rn01455646_m1 (ThermoFisher, Waltham MA).

5.2.2 Determination of Skeletal Muscle HMGCR mRNA Abundance

RNA extraction was performed using RNeasy fibrous tissue mini kit protocol (Qiagen, Germany). Briefly, a 30 mg piece of frozen skeletal muscle was lysed and homogenized using a Tissue Lyser II (Qiagen, Germany) with one stainless steel bead and lysis reagent containing P-mercaptoethanol. Next, proteinase k was added. RNA was bound, washed, and eluted from RNEasy mini spin columns. The optional on-column DNase digestion was performed using the RNase free DNase set (Qiagen, Germany). A total of 40 μL of RNA was eluted from each sample. RNA samples were quantified using NanoDrop 2000. Quantified RNA samples were diluted and then reverse transcribed using SuperScript Vilo Master mix (ThermoFisher, Waltham MA). Taqman qPCR was performed with the cDNA samples using Taqman gene expression assays Rn00565598_m1 and Rn01455646_m1 (ThermoFisher, Waltham MA).

5.2.3 Incorporation of Guide Strand into Liver or Skeletal Muscle RNA-Silencing Complex

### Tissue Lysis:

Each frozen tissue piece provided in screw cap cryo tube was transferred to a 2 mL round bottom microcentrifuge tube that was pre-chilled on dry ice. One dry ice pre-chilled 5 mm stainless steel bead (Qiagen, Germany) was added to the tube containing the frozen tissue. In the cold room, sample tubes were quickly removed from dry ice and added to each tube 1 mL of ice-cold Lysis buffer (50 mM Tris-HCl pH 7.5, 150 mM NaCl, 2 mM EDTA, 0.5% Triton X-100, 1 mM PMSF, 1×EDTA-free protease inhibitor cocktail). Immediately, tissue was lysed with TissueLyser LT (Qiagen, Germany) for 5 min at 50 Hz in cold room. Lysate was then cleared at 20000×g, 10 min, 4° C., and the soluble lysate supernatants were kept on ice. The protein concentration of the soluble lysate for each sample was determined using BCA assay (ThermoFisher, Waltham MA) according to manufacturer's protocol.

### Argonaute 2 Immunoprecipitation (IP):

Dynabead Protein G (ThermoFisher, Waltham MA) was washed with Wash buffer (50 mM Tris-HCl pH 7.5, 150 mM NaCl, 2 mM EDTA, 0.5% Triton X-100) prior to use for IP, and 50 μL of bead slurry was used per sample. Mouse argonaute2 (Ago2) antibody (FUJIFILM Wako Chemicals, Richmond VA) was pre-bound to beads in wash buffer at 4° C., for 2 h on rotating mixer (200 ng antibody used per 50 μL bead slurry). After incubation, the Ago2 antibody-bound beads were washed with Wash buffer, and 50 μL of the suspension was distributed to a 1.5 mL microcentrifuge tube per sample. For each sample, Ago2 antibody-bound beads were incubated with 500 μg soluble tissue lysate in lysis buffer at final volume of 250 μL per sample at 4° C., overnight on rotating mixer. After incubation, beads in each tube were washed 5 times with 1 mL ice cold Wash buffer. Final resuspension of beads was with 50 μL PBST (phosphate buffered saline pH 7.4, 0.25% Triton X-100) per sample. siRNAs were released from bead by heating at 95° C., 5 min. Ago2 IP eluate supernatants were recovered and kept on ice on the same day or stored at −80° C. until the subsequent stem loop-reverse transcription quantitative polymerase chain reaction (SL-RT-qPCR) step.

siRNA Standard Preparation:

siRNA was first diluted to a working stock of 10 ng/μL in H2O, then further diluted 100-fold to 100 ng/mL in PBST. siRNA standards were prepared by 10-fold serial dilution from 100 ng/mL to 0.00001 ng/mL in PBST.

### Stem Loop-Reverse Transcription Quantitative Polymerase Chain Reaction (SL-RT-qPCR):

For SL-RT-qPCR, Custom Small RNA Assay (4398987, ThermoFisher, Waltham MA) containing a set of SL-RT primer and Taqman qPCR primer against guide strand sequence of Compound 7 was designed and ordered. cDNA was generated following manufacturer's protocol of the Taqman MicroRNA Reverse Transcription Kit (ThermoFisher, Waltham MA), using 5 μL of Ago2 IP eluate or 5 μL siRNA standard. The cDNA generated (15 μL reaction) were then diluted with 75 μL H2O prior to usage for qPCR step. qPCR was performed following manufacturer's protocol for TaqMan™ Fast Advanced Master Mix (ThermoFisher, Waltham MA), using 4 μL of the diluted cDNA.

### Calculations:

An siRNA standard curve was generated by plotting Ct values (Y) versus siRNA concentration (X) in log scale using GraphPad Prism (version 9.4.1), followed by semi-log line fitting to determine slope and y-intercept values. siRNA concentration for each sample was calculated using the obtained Ct value and the determined slope and y-intercept values.

$$ng siRNA = [ siRNA ] \times Ago 2 IP elution volume ( 50 μ L )$$

$$ng siRNA per g soluble lysate protein = ng siRNA per 500 μ g ( amount used in Ago 2 IP )$$

### Data Analysis:

Statistical significance was determined by ordinary one-way ANOVA and Dunnett's multiple comparisons test using GraphPad Prism software (version 9.4.1).

### Example 6: HMGCR siRNA Increases LDLR Protein in Human Liver Cells

### 6.1 Primary Human Hepatocyte Culture

Cryopreserved 999Elite primary human hepatocytes (PHH, Lot 1142) were obtained from Discovery Life Sciences (Huntsville, AL), and registered in the Novartis Human Biological Sample tracking system. Thawing, counting, and plating of hepatocytes was conducted following the vendor's protocol. Briefly, cells were seeded using medium UPCM into 48-well (0.15 million/0.4 mL/well) or 24-well plates (0.32 million/0.8 mL/well) for 4h incubation in 37° C. cell culture incubator supplied with 95% 02/5% CO2. The medium was then switched to hepatocyte induction medium (HIM; DLS, Cat #81018: 0.25 mL/well, 48-well plate; 0.5 mL/well, 24-well plate) for 30 min of incubation prior to siRNA transfection or compound treatment.

### 6.2 Human Hepatoma Cell Line Huh7

Human hepatoma cell line Huh7 was obtained from ATCC. Cells were maintained in pyruvate-free DMEM (Thermo Fisher Scientific, Waltham, MA, Cat #11965-092) supplemented with 10% heat-inactivated fetal bovine serum (FBS, Thermo Fisher Scientific, Cat #10082-147). Huh7 cells (passage number 14) were split using 0.25% Trypsin/2.21 mM EDTA (Corning Life Sciences (Corning, NY), Cat #25-053-CI), seeded into 6-well plates (0.21 million/2.5 mL complete DMSM/well) for 18 h growth in a cell culture incubator. The medium was next switched to Opti-MEM (Thermo Fisher Scientific, Cat #31985062; 2.5 mL/well) for 30 min incubation prior to siRNA transfection or compound treatment.

6.3 siRNA Transfection

siRNA agents were complexed to Lipofectamine RNAiMAX (Thermo Fisher Scientific, Cat #13778150) in Opti-MEM (Thermo Fisher Scientific, Cat #31985062) according to the vendor's manual. Briefly, the siRNA-to-Lipofectamine ratio was 33.3 μmole:1 μL, with siRNA as 600 nM in the complex stock. siRNA/Lipofectamine complex was added to HIM (for PHH) or Opti-MEM (for Huh7) medium to achieve a final siRNA concentration of 10 nM or 25 nM, respectively. The PHH culture was set for 24h transfection without a change of medium. The Huh7 culture was incubated with siRNA for 6 h at 37° C., then refreshed with DMEM/10% FBS (2.5 mL/well, 6-well plate) for an additional 24h incubation. Negative control siRNA (Thermo Fisher Scientific, Cat #: 4390847) and in-house HMGCR siRNA (Compound 1) were used for siRNA transfection.

### 6.4 Statin Treatment

In PHH experiments, atorvastatin (25 μM in DMSO) was diluted in HIM medium to achieve a concentration of 10 nM. Then pre-existing HIM was replaced with an equal volume of HIM/atorvastatin (0.25 mL/well, 48-well pate; 0.5 mL/well, 24-well plate) for a 24h incubation. In Huh7 experiments, there was first a 6h incubation with Opti-MEM in parallel of 6h siRNA incubation. Then Opti-MEM was replaced with DMEM/10%/25 nM atorvastatin (2.5 mL/well) for 24h incubation. In both cell culture systems, DMSO was added to medium to serve as a control.

6.5 mRNA Quantification

After siRNA transfection or atorvastatin treatment, cells were washed twice with PBS, lysed with TRK lysis buffer (Total RNA kit, Cat #R6834-02, Omega Bio-Tek (Norcross, GA)) for RNA isolation. RNA was quantified using a NanoDrop 2000 (Thermo Fisher Scientific). cDNA was synthesized using a TaqMan Reverse Transcription Kit (Thermo Fisher Scientific, Cat #N8080234). Briefly, the RNA content in cDNA synthesis was 10 ng/μL. cDNA was then diluted 1:6 in water and subjected to Real-Time PCR assays using FAM/MGB probes for human TBP (hs00427620_m1, Thermo Fisher Scientific), HPRT1 (Hs02800695_m1), PPIA (Hs99999904_m1), HMGCR (Hs00168352_m1), and LDLR (Hs01092524_m1). cDNA input was 4 μL in each 10 μL PCR setup using Taqman Universal PCR Master Mix (Thermo Fisher Scientific, Cat #4304437). PCR assays were run in Quant Studio 5 with Design and Analysis Software v1.5.2. For Huh7 culture, target gene mRNA was normalized to house-keeping gene TBP based on Ct values derived by PCR. For PHH culture, Ct geomean was generated from 3 house-keeping genes (TBP, HPRT1, PPIA), for target gene normalization. In all cell culture controls, target mRNA expression was arbitrarily assigned a value of 1. mRNA expression in treatment group(s) was calculated as a fold-change relative to control.

### 6.6 Western Blotting

After treatment, cells were washed twice with PBS and lysed with 1×NuPAGE LDS sample buffer/1× reducing agent (Thermo Fisher Scientific: Cat #NP0007, NP0009). The volume of sample buffer was 100 μL/well (24-well plate) or 500 μL/well (6-well plate), respectively. Cellular lysate samples were subject to sonication (25 sec×3, output level=6, MICROSON Ultrasonic Cell Disruptor, MISONIX Inc. (Farmingdale, NY). Cellular proteins were resolved in NuPAGE 4-12% Bis-Tris gel (Thermo Fisher Scientific, NP02323 Box) and transferred onto nitrocellulose membrane in an iBlot2 Dry Blotting System (Thermo Fisher Scientific). Proteins were probed using rabbit monoclonal antibody for GAPDH (Cat #5174S, 1:4500; Cell Signaling Technology, Danvers, MA) or rabbit polyclonal antibody for human LDL receptor (Cat #LS-C146979, 1:1000, LifeSpan Bioscience, Shirley MA). Primary antibodies were detected using HRP-conjugated Ab (goat anti-rabbit IgG, Cat ##7074S, 1:2500; or goat anti-mouse IgG, Cat #7076S, 1:2500. Cell Signaling Technology). Protein bands were visualized using a SuperSignal West Femto Kit (Thermo Fisher Scientific, Cat #34096), with images captured using Amersham Imager 680 (GE Healthcare) and quantified using ImageQuant TL Software. Densitometric values for target protein were normalized to that of GAPDH. Target protein level for the control was arbitrarily set as 1, therefore protein expression in treatment group(s) represents fold-change relative to control.

### Example 7: HMGCR siRNA Reduces HMGCR Protein in Huh7 Cells

### 7.1 Human Hepatoma Cell Line Huh7

Human hepatoma cell line Huh7 was obtained from ATCC. Cells were maintained in pyruvate-free DMEM (Thermo Fisher Scientific, Waltham, MA, Cat #11965-092) supplemented with 10% heat-inactivated fetal bovine serum (FBS, Thermo Fisher Scientific, Cat #10082-147). Huh7 cells (passage number 14) were split using 0.25% Trypsin/2.21 mM EDTA (Corning Life Sciences (Corning, NY), Cat #25-053-CI), seeded into 6-well plates (0.21 million/2.5 mL complete DMSM/well) for 18 h growth in a cell culture incubator. The medium was next switched to Opti-MEM (Thermo Fisher Scientific, Cat #31985062; 2.5 mL/well) for 30 min incubation prior to siRNA transfection or compound treatment.

7.2 siRNA Transfection

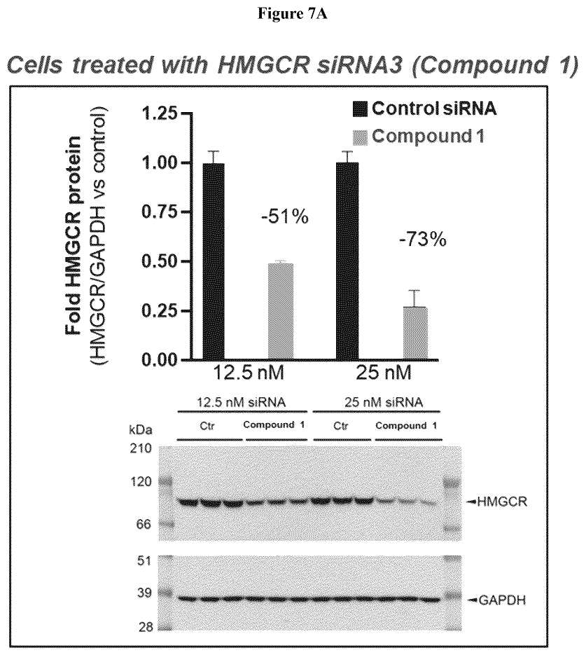

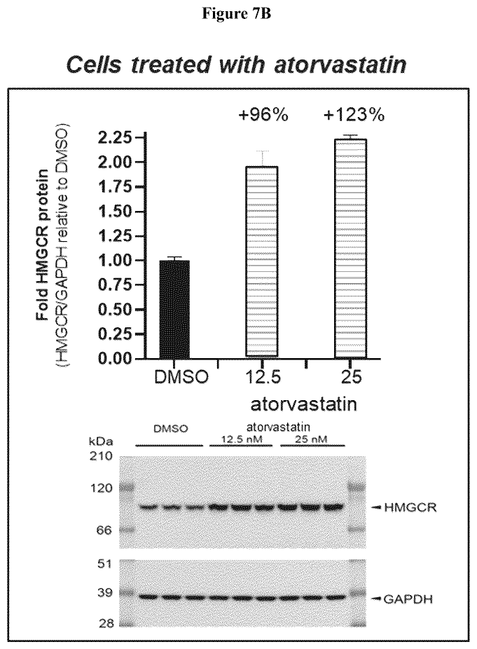

siRNA agents were complexed to Lipofectamine RNAiMAX (Thermo Fisher Scientific, Cat #13778150) in Opti-MEM (Thermo Fisher Scientific, Cat #31985062) according to the vendor's manual. Briefly, the siRNA-to-Lipofectamine ratio was 33.3 pmole:1 μL, with siRNA as 600 nM in the complex stock. siRNA/Lipofectamine complex was added to Opti-MEM medium to achieve a final siRNA concentration of 12.5 nM or 25 nM (FIG. 7A). The Huh7 culture was incubated with siRNA for 6 h at 37° C., then refreshed with DMEM/10% FBS (2.5 mL/well, 6-well plate) for an additional 24h incubation. Negative control siRNA (Thermo Fisher Scientific, Cat #: 4390847) or HMGCR siRNA 3 (Compound 1) were used for siRNA transfection.

### 7.3 Statin Treatment

Huh7 cells were incubated with Opti-MEM for 6h. This was followed by replacement of Opti-MEM with DMEM/10%/12.5 or 25 nM atorvastatin (2.5 mL/well) and incubation for 24h (FIG. 7B). DMSO was added to medium to serve as a control.

### 7.4 Western Blotting

After treatment, cells were washed twice with PBS and lysed with 1×NuPAGE LDS sample buffer/1× reducing agent (Thermo Fisher Scientific: Cat #NP0007, NP0009). The volume of sample buffer was 100 μL/well (24-well plate) or 500 μL/well (6-well plate), respectively. Cellular lysate samples were subject to sonication (25 sec×3, output level=6, MICROSON Ultrasonic Cell Disruptor, MISONIX Inc. (Farmingdale, NY). Cellular proteins were resolved in NuPAGE 4-12% Bis-Tris gel (Thermo Fisher Scientific, NP02323 Box) and transferred onto nitrocellulose membrane in an iBlot2 Dry Blotting System (Thermo Fisher Scientific). Proteins were probed using rabbit monoclonal antibody for GAPDH (Cat #5174S, 1:4500; Cell Signaling Technology, Danvers, MA) or rabbit polyclonal antibody for human LDL receptor (Cat #LS-C146979, 1:1000, LifeSpan Bioscience, Shirley MA). Primary antibodies were detected using HRP-conjugated Ab (goat anti-rabbit IgG, Cat ##7074S, 1:2500; or goat anti-mouse IgG, Cat #7076S, 1:2500. Cell Signaling Technology). Protein bands were visualized using a SuperSignal West Femto Kit (Thermo Fisher Scientific, Cat #34096), with images captured using Amersham Imager 680 (GE Healthcare) and quantified using ImageQuant TL Software. Densitometric values for target protein were normalized to that of GAPDH. Target protein level for the control was arbitrarily set as 1, therefore protein expression in the treatment group(s) represents fold-change relative to control.

### 7.5 Results

Relative to the control siRNA, Compound 1 reduced HMGCR protein content by 51% and 73% at concentrations of 12.5 and 25 nM, respectively (FIG. 7A). These results demonstrate that Compound 1 significantly, and dose-dependently, reduces HMGCR protein content in a human liver cell line.

In contrast, relative to DMSO, atorvastatin markedly augmented HMGCR protein levels, with 96% and 123% increases observed relative to control at concentrations of 12.5 and 25 nM, respectively (FIG. 7B). These results demonstrate that, in contrast to Compound 1, atorvastatin robustly increases HMGCR protein expression in a human liver cell line.

### Example 8: In Vitro Screening

Extended siRNA sequences were designed to be complementary to human HMGCR isoform, transcript variant 1, mRNA (GenBank: NM_000859.3) and about four hundreds of RNAi agents (siRNAs) to HMGCR were screened and tested for off-target hybridization (e.g., less off-target hybridization) and knock-down of HMGCR mRNA in Hepa-1-6-cells and HEK293 cells for evaluating off-target profiling.

### 8.1 Assays Using Reporter in Hepa-1-6 Cells

Hepa-1-6 cells (ATCC, cat. CRL-1830) were cultured in DMEM (ATCC, cat. 30-2002) medium containing 10% FBS at 37 C with 5% CO2. Cells were seeded in a 96-well plate and reverse transfected using lipofectamine (Thermo, Liopfecatmine 2000, cat. 11668019) with 10Ong/well psiCheck2 (Promega, cat. C8021) containing an insert for HMGCR (NM_000859.3) in combination with siRNAs starting from 50 nM using a 1:4 dilution factor in a 10-point dose response as indicated in respective figures and tables. Firefly and renilla luciferase signals were obtained using Dual-Glo® Stop & Glo® Reagent (Promega, cat. E2940). The degree of knockdown was calculated from the ratio of renilla luminescence signal to firefly luminescence signal.

The results of suppression by example HMGCR siRNA agents in Hepa-1-6 cells are shown in Table 7.

TABLE 7
Suppression in HMGCR protein expression in Hepa-1-6

| | | | | 12.5 nM | | 3.125 nM | | 0.78125 nM | | |
|---|---|---|---|---|---|---|---|---|---|---|
| | | 50 nM siRNA | | siRNA | | siRNA | | siRNA | | |
| | position | (%) | | (%) | | (%) | | (%) | | |
| siRNA | in | protein | (%) | protein | (%) | protein | (%) | protein | (%) | IC₈₀ |
| No. | mRNA | expression | SD | expression | SD | expression | SD | expression | SD | (nM) |
| 408 | 126 | 4.07 | 0.03 | 3.43 | 0.08 | 3.85 | 0.12 | 6.14 | 0.12 | 0.13 |
| 409 | 127 | 2.99 | 0.13 | 2.89 | 0.20 | 3.07 | 0.08 | 5.14 | 0.33 | 0.10 |
| 411 | 131 | 6.81 | 0.19 | 4.45 | 0.28 | 4.56 | 0.34 | 6.06 | 0.44 | 0.12 |
| 412 | 133 | 10.57 | 0.35 | 8.05 | 0.48 | 8.66 | 0.76 | 11.90 | 0.60 | 0.22 |
| 415 | 277 | 4.87 | 0.12 | 4.08 | 0.22 | 4.13 | 0.02 | 4.74 | 0.06 | 0.13 |
| 417 | 313 | 7.97 | 0.11 | 6.21 | 0.10 | 7.30 | 0.09 | 9.02 | 0.35 | 0.16 |
| 420 | 318 | 10.96 | 1.20 | 9.18 | 0.57 | 10.01 | 0.60 | 13.16 | 0.53 | 0.23 |
| 425 | 366 | 8.17 | 0.68 | 6.52 | 0.30 | 6.90 | 0.36 | 8.32 | 0.56 | 0.19 |
| 427 | 371 | 6.54 | 0.19 | 6.11 | 0.19 | 6.02 | 0.45 | 7.00 | 0.20 | 0.06 |
| 445 | 683 | 25.26 | 1.30 | 25.14 | 0.51 | 26.63 | 1.18 | 30.41 | 0.06 | 0.67 |
| 453 | 739 | 20.94 | 1.03 | 20.34 | 0.97 | 24.61 | 0.29 | 27.47 | 0.97 | 0.27 |
| 464 | 967 | 25.41 | 1.38 | 25.81 | 1.07 | 26.62 | 1.61 | 30.49 | 1.44 | 0.20 |
| 465 | 968 | 28.00 | 0.82 | 25.55 | 1.27 | 27.53 | 1.58 | 30.07 | 1.03 | 0.24 |
| 466 | 972 | 30.52 | 1.03 | 30.76 | 1.50 | 28.31 | 1.11 | 32.28 | 0.87 | 0.26 |
| 467 | 1083 | 27.50 | 1.62 | 24.40 | 0.74 | 24.66 | 0.11 | 27.36 | 1.66 | 0.17 |
| 487 | 1328 | 33.31 | 1.48 | 31.39 | 0.31 | 37.63 | 1.81 | 47.58 | 1.08 | 1.05 |
| 489 | 1357 | 35.87 | 1.47 | 32.03 | 0.22 | 35.44 | 1.88 | 44.06 | 2.72 | 0.71 |
| 491 | 1381 | 38.72 | 0.56 | 31.56 | 0.59 | 35.61 | 2.05 | 44.59 | 0.45 | 0.63 |
| 498 | 1521 | 27.60 | 2.27 | 24.47 | 1.16 | 25.47 | 1.03 | 27.18 | 1.08 | 0.13 |
| 502 | 1531 | 21.58 | 0.26 | 20.06 | 0.50 | 21.64 | 0.62 | 23.91 | 0.69 | 0.16 |
| 511 | 1567 | 30.87 | 0.48 | 27.18 | 0.13 | 28.72 | 1.46 | 37.56 | 1.74 | 0.48 |
| 514 | 1583 | 33.54 | 0.10 | 30.69 | 0.93 | 33.47 | 0.59 | 39.06 | 1.90 | 0.57 |
| 516 | 1587 | 24.04 | 1.65 | 23.44 | 0.15 | 23.75 | 0.34 | 28.33 | 1.77 | 0.16 |
| 533 | 1953 | 35.00 | 0.68 | 29.87 | 0.26 | 32.87 | 1.37 | 41.37 | 2.26 | 0.58 |
| 545 | 2007 | 29.51 | 1.08 | 24.34 | 0.92 | 25.78 | 1.15 | 30.41 | 0.81 | 0.19 |
| 547 | 2042 | 41.33 | 1.57 | 35.03 | 0.75 | 36.39 | 0.16 | 49.26 | 2.20 | 0.85 |
| 555 | 2266 | 31.20 | 2.45 | 26.73 | 0.55 | 26.94 | 0.96 | 27.98 | 0.27 | 0.11 |
| 557 | 2274 | 22.65 | 1.21 | 23.03 | 0.28 | 23.37 | 1.07 | 26.75 | 0.51 | 0.38 |
| 577 | 2399 | 39.41 | 0.22 | 34.70 | 2.70 | 38.65 | 0.51 | 50.62 | 0.38 | 1.68 |
| 578 | 2424 | 29.88 | 0.69 | 32.32 | 1.85 | 34.96 | 0.24 | 46.43 | 1.52 | 1.90 |
| 579 | 2426 | 27.66 | 1.64 | 26.01 | 1.31 | 28.06 | 1.60 | 29.25 | 0.24 | 0.31 |
| 580 | 2429 | 29.69 | 0.14 | 24.86 | 0.13 | 25.97 | 0.74 | 28.19 | 1.23 | 0.11 |
| 583 | 2484 | 8.17 | 0.24 | 5.94 | 0.27 | 5.90 | 0.62 | 6.51 | 0.31 | 0.14 |
| 585 | 2575 | 16.25 | 2.30 | 14.82 | 0.91 | 15.53 | 0.45 | 29.44 | 3.89 | 1.10 |
| 588 | 2592 | 27.42 | 0.11 | 24.18 | 2.09 | 24.79 | 1.83 | 33.95 | 4.68 | 0.95 |
| 591 | 2627 | 50.90 | 3.00 | 41.09 | 3.48 | 43.79 | 4.46 | 47.64 | 4.69 | 0.34 |
| 597 | 2723 | 14.03 | 0.96 | 9.12 | 0.07 | 8.52 | 0.16 | 9.51 | 0.65 | 0.05 |
| 601 | 2731 | 17.50 | 0.37 | 15.00 | 1.28 | 15.62 | 0.60 | 15.32 | 0.36 | 0.03 |
| 603 | 2734 | 22.44 | 0.95 | 16.30 | 1.39 | 14.06 | 0.49 | 21.93 | 2.20 | 0.43 |
| 604 | 2735 | 22.34 | 1.48 | 9.36 | 1.08 | 8.09 | 0.37 | 10.56 | 0.74 | 0.00 |
| 611 | 2994 | 10.96 | 0.87 | 9.39 | 0.09 | 9.69 | 0.05 | 12.54 | 0.31 | 0.10 |
| 613 | 3071 | 43.86 | 2.58 | 28.14 | 0.66 | 22.97 | 1.04 | 27.92 | 2.08 | 0.08 |
| 614 | 3102 | 8.48 | 0.90 | 7.04 | 0.21 | 7.24 | 0.46 | 8.43 | 0.30 | 0.13 |
| 616 | 3105 | 7.54 | 0.70 | 5.41 | 0.22 | 6.32 | 0.72 | 8.32 | 0.61 | 0.23 |
| 620 | 3126 | 11.81 | 0.80 | 9.93 | 0.52 | 10.39 | 1.21 | 13.26 | 1.48 | 0.20 |
| 621 | 3129 | 15.00 | 0.55 | 10.61 | 0.37 | 11.76 | 0.36 | 15.94 | 1.37 | 0.22 |
| 622 | 3130 | 10.54 | 0.82 | 10.89 | 1.33 | 9.57 | 0.74 | 11.59 | 0.24 | 0.05 |
| 633 | 3484 | 31.49 | 2.21 | 23.55 | 1.77 | 23.04 | 1.43 | 27.88 | 4.37 | 0.21 |

### 8.2 HEK293T Cell Assay

Adherent HEK293T cells (ATCC, cat. CRL-3216) were cultured as recommended by the manufacture. Cells were transfected 24 hrs post-seeding using lipofectamine (Invitrogen, Lipofectamine™ RNAiMax 13778150) in a 96-well plate. Cells were lysed using FastLance (Qiagen, cat. 216713) and followed manufacturer protocol for RT-qPCR using specific primer probes for HMGCR (Thermo, Hs00168352_m1) which was normalized to the house keeping gene PGK (Thermo, Hs9999906_m1). Changes in expression were calculated using delta-delta CT method.

siRNAs for transfection are listed in Table 8.

TABLE 8

| | position | | SEQ | | SEQ |
|---|---|---|---|---|---|
| siRNA | in | | ID | | ID |
| No. | mRNA | Sense Strand | NO: | Antisense strand | NO: |
| 655 | 126 | U007p001U004p001G007pU004 | 1310 | U004p001U007p001C004pG007p | 1357 |
| | | pC007pA004pA007pG004pA007 | | A004pA007pA004pA007pA004pG | |
| | | pC004pU007pU004pU007pU004 | | 007pU004pC007pU004pU007pG0 | |
| | | pU007pC004pG007pA004pA007 | | 04pA007pC004pA007pA004p001 | |
| | | pX2000 | | U004p001U004 | |
| 656 | 131 | G007p001A004p001U007pU004 | 1311 | A004p001G007p001A004pC007p | 1358 |
| | | pG007pG004pC007pU004pA007 | | A004pU007pG004pU007pU004pA | |
| | | pU004pA007pA004pC007pA004 | | 007pU004pA007pG004pC007pC0 | |
| | | pU007pG004pU007pC004pU007 | | 04pA007pA004pU007pC004p001 | |
| | | pX2000 | | U004p001U004 | |
| 657 | 277 | G007p001A004p001A007pG004 | 1312 | U004p001U007p001A004pU007p | 1359 |
| | | pG007pG004pU007pU004pC007 | | C004pA007pC004pU007pG004pC | |
| | | pG004pC007pA004pG007pU004 | | 007pG004pA007pA004pC007pC0 | |
| | | pG007pA004pU007pA004pA007 | | 04pC007pU004pU007pC004p001 | |
| | | px2000 | | U004p001U004 | |
| 658 | 295 | U007p001G004p001A007pG004 | 1313 | A004p001U007p001U004pA007p | 1360 |
| | | pc007pA004pG007pU004pG007 | | U004pA007pA004pU007pG004pU | |
| | | pA004pC007pA004pU007pU004 | | 007pC004pA007pC004pU007pG0 | |
| | | pA007pU004pA007pA004pU007 | | 04pC007pU004pC007pA004p001 | |
| | | pX2000 | | U004p001U004 | |
| 659 | 445 | C007p001A004p001G007pU004 | 1314 | A004p001A007p001G004pA007p | 1361 |
| | | pu007pG004pU007pC004pA007 | | A004pG007pU004pG007pA004pA | |
| | | pU004pU007pC004pA007pC004 | | 007pU004pG007pA004pC007pA0 | |
| | | pu007pU004pC007pU004pU007 | | 04pA007pC004pU007pG004p001 | |
| | | pX2000 | | U004p001U004 | |
| 660 | 730 | C007p001C004p001A007pA004 | 1315 | A004p001U007p001G004pA007p | 1362 |
| | | pc007pU004pA007pC004pU007 | | A004pC007pA004pC007pG004pA | |
| | | pU004pC007pG004pU007pG004 | | 007pA004pG007pU004pA007pG0 | |
| | | pU007pU004pC007pA004pU007 | | 04pU007pU004pG007pG004p001 | |
| | | px2000 | | U004p001U004 | |
| 661 | 736 | A007p001C004p001U007pU004 | 1316 | A004p001A007p001A004pG007p | 1363 |
| | | pC007pG004pU007pG004pU007 | | U004pC007pA004pU007pG004pA | |
| | | pU004pC007pA004pU007pG004 | | 007pA004pC007pA004pC007pG0 | |
| | | pA007pC004pU007pU004pU007 | | 04pA007pA004pG007pU004p001 | |
| | | pX2000 | | U004p001U004 | |
| 662 | 739 | U007p001C004p001G007pU004 | 1317 | A004p001A007p001G004pA007p | 1364 |
| | | pG007pU004pU007pC004pA007 | | A004pA007pG004pU007pC004pA | |
| | | pU004pG007pA004pC007pU004 | | 007pU004pG007pA004pA007pC0 | |
| | | pU007pU004pC007pU004pU007 | | 04pA007pC004pG007pA004p001 | |
| | | pX2000 | | U004p001U004 | |
| 663 | 1328 | C007p001U004p001U007pA004 | 1318 | A004p001U007p001C004pU007p | 1365 |
| | | pG007pU004pG007pG004pC007 | | G004pU007pU004pU007pC004pA | |
| | | pU004pG007pA004pA007pA004 | | 007pG004pC007pC004pA007pC0 | |
| | | pc007pA004pG007pA004pU007 | | 04pU007pA004pA007pG004p001 | |
| | | pX2000 | | U004p001U004 | |
| 664 | 1516 | C007p001U004p001G007pA004 | 1319 | A004p001C007p001U004pA007p | 1366 |
| | | pG007pA004pU007pC004pA007 | | A004pC007pU004pG007pG004pA | |
| | | pU004pC007pC004pA007pG004 | | 007pU004pG007pA004pU007pC0 | |
| | | pU007pU004pA007pG004pU007 | | 04pU007pC004pA007pG004p001 | |
| | | px2000 | | U004p001U004 | |
| 665 | 1555 | C007p001C004p001U007pA004 | 1320 | A004p001G007p001A004pG007p | 1367 |
| | | pc007pA004pA007pG004pU007 | | U004pU007pU004pC007pC004pA | |
| | | pU004pG007pG004pA007pA004 | | 007pA004pC007pU004pU007pG0 | |
| | | pA007pC004pU007pC004pU007 | | 04pU007pA004pG007pG004p001 | |
| | | pX2000 | | U004p001U004 | |
| 666 | 1558 | A007p001C004p001A007pA004 | 1321 | A004p001U007p001C004pA007p | 1368 |
| | | pG007pU004pU007pG004pG007 | | G004pA007pG004pU007pU004pU | |
| | | pA004pA007pA004pC007pU004 | | 007pC004pC007pA004pA007pC0 | |
| | | pC007pU004pG007pA004pU007 | | 04pU007pU004pG007pU004p001 | |
| | | px2000 | | U004p001U004 | |
| 667 | 1586 | U007p001G004p001A007pG004 | 1322 | A004p001A007p001U004pA007p | 1369 |
| | | pC007pG004pU007pG004pG007 | | G004pA007pU004pA007pC004pA | |
| | | pU004pG007pU004pA007pU004 | | 007pC004pC007pA004pC007pG0 | |
| | | pc007pU004pA007pU004pU007 | | 04pC007pU004pC007pA004p001 | |
| | | pX2000 | | U004p001U004 | |
| 668 | 1996 | C007p001U004p001A007pG004 | 1323 | A004p001G007p001A004pC007p | 1370 |
| | | pC007pA004pG007pA004pU007 | | G004pU007pG004pC007pA004pA | |
| | | pU004pU007pG004pC007pA004 | | 007pA004pU007pC004pU007pG0 | |
| | | pC007pG004pU007pC004pU007 | | 04pC007pU004pA007pG004p001 | |
| | | pX2000 | | U004p001U004 | |
| 669 | 2169 | G007p001U004p001U007pA004 | 1324 | U004p001A007p001C004pA007p | 1371 |
| | | pG007pU004pG007pG004pU007 | | A004pU007pA004pG007pU004pU | |
| | | pA004pA007pC004pU007pA004 | | 007pA004pC007pC004pA007pC0 | |
| | | pU007pU004pG007pU004pA007 | | 04pU007pA004pA007pC004p001 | |
| | | pX2000 | | U004p001U004 | |
| 670 | 2269 | U007p001U004p001G007pU004 | 1325 | U004p001U007p001U004pA007p | 1372 |
| | | pC007pA004pG007pA004pG007 | | A004pU007pA004pC007pU004pU | |
| | | pA004pA007pG004pU007pA004 | | 007pC004pU007pC004pU007pG0 | |
| | | pU007pU004pA007pA004pA007 | | 04pA007pC004pA007pA004p001 | |
| | | pX2000 | | U004p001U004 | |
| 671 | 2274 | A007p001G004p001A007pG004 | 1326 | U004p001A007p001G004pU007p | 1373 |
| | | pA007pA004pG007pU004pA007 | | C004pU007pU004pU007pA004pA | |
| | | pU004pU007pA004pA007pA004 | | 007pU004pA007pC004pU007pU0 | |
| | | pG007pA004pC007pU004pA007 | | 04pC007pU004pC007pU004p001 | |
| | | pX2000 | | U004p001U004 | |
| 672 | 2424 | G007p001C004p001A007pG004 | 1327 | U004p001A007p001C004pC007p | 1374 |
| | | pC007pA004pC007pA004pG007 | | A004pA007pC004pA007pU004pU | |
| | | pA004pA007pU004pG007pU004 | | 007pC004pU007pG004pU007pG0 | |
| | | pU007pG004pG007pU004pA007 | | 04pC007pU004pG007pC004p001 | |
| | | px2000 | | U004p001U004 | |
| 673 | 2484 | A007p001A004p001U007pG004 | 1328 | U004p001G007p001A004pU007p | 1375 |
| | | pA007pA004pG007pA004pU007 | | A004pU007pA004pU007pA004pA | |
| | | pU004pU007pA004pU007pA004 | | 007pA004pU007pC004pU007pU0 | |
| | | pU007pA004pU007pC004pA007 | | 04pC007pA004pU007pU004p001 | |
| | | pX2000 | | U004p001U004 | |
| 674 | 2575 | U007p001G004p001C007pA004 | 1329 | U004p001G007p001A004pA007p | 1376 |
| | | pG007pA004pU007pG004pC007 | | C004pA007pC004pC007pU004pA | |
| | | pU004pA007pG004pG007pU004 | | 007pG004pC007pA004pU007pC0 | |
| | | pG007pU004pU007pC004pA007 | | 04pU007pG004pC007pA004p001 | |
| | | pX2000 | | U004p001U004 | |
| 675 | 2576 | G007p001C004p001A007pG004 | 1330 | U004p001U007p001G004pA007p | 1377 |
| | | pA007pU004pG007pC004pU007 | | A004pC007pA004pC007pC004pU | |
| | | pA004pG007pG004pU007pG004 | | 007pA004pG007pC004pA007pU0 | |
| | | pU007pU004pC007pA004pA007 | | 04pC007pU004pG007pC004p001 | |
| | | pX2000 | | U004p001U004 | |
| 676 | 2592 | C007p001A004p001A007pG004 | 1331 | U004p001A007p001U004pC007p | 1378 |
| | | pG007pA004pG007pC004pA007 | | U004pU007pU004pG007pC004pA | |
| | | pU004pG007pC004pA007pA004 | | 007pU004pG007pC004pU007pC0 | |
| | | pA007pG004pA007pU004pA007 | | 04pC007pU004pU007pG004p001 | |
| | | pX2000 | | U004p001U004 | |
| 677 | 2627 | C007p001C004p001G007pG004 | 1332 | A004p001A007p001U004pU007p | 1379 |
| | | pC007pA004pG007pC004pU007 | | C004pG007pG004pG007pC004pA | |
| | | pU004pG007pC004pC007pC004 | | 007pA004pG007pC004pU007pG0 | |
| | | pG007pA004pA007pU004pU007 | | 04pC007pC004pG007pG004p001 | |
| | | pX2000 | | U004p001U004 | |
| 678 | 2728 | A007p001C004p001A007pA004 | 1333 | U004p001U007p001G004pA007p | 1380 |
| | | pC007pA004pG007pG004pU007 | | U004pC007pU004pU007pC004pG | |
| | | pC004pG007pA004pA007pG004 | | 007pA004pC007pC004pU007pG0 | |
| | | pA007pU004pC007pA004pA007 | | 04pU007pU004pG007pU004p001 | |
| | | pX2000 | | U004p001U004 | |
| 679 | 2731 | A007p001C004p001A007pG004 | 1334 | A004p001A007p001A004pU007p | 1381 |
| | | pG007pU004pC007pG004pA007 | | U004pG007pA004pU007pC004pU | |
| | | pA004pG007pA004pU007pC004 | | 007pU004pC007pG004pA007pC0 | |
| | | pA007pA004pU007pU004pU007 | | 04pC007pU004pG007pU004p001 | |
| | | px2000 | | U004p001U004 | |
| 680 | 2737 | C007p001G004p001A007pA004 | 1335 | U004p001C007p001U004pU007p | 1382 |
| | | pG007pA004pU007pC004pA007 | | G004pU007pA004pA007pA004pU | |
| | | pA004pU007pU004pU007pA004 | | 007pU004pG007pA004pU007pC0 | |
| | | pC007pA004pA007pG004pA007 | | 04pU007pU004pC007pG004p001 | |
| | | pX2000 | | U004p001U004 | |
| 681 | 2937 | A007p001G004p001A007pG004 | 1336 | A004p001A007p001G004pA007p | 1383 |
| | | pA007pG004pG007pU004pC007 | | A004pC007pC004pU007pG004pA | |
| | | pU004pC007pA004pG007pG004 | | 007pG004pA007pC004pC007pU0 | |
| | | pu007pU004pC007pU004pU007 | | 04pC007pU004pC007pU004p001 | |
| | | pX2000 | | U004p001U004 | |
| 682 | 3108 | U007p001G004p001A007pU004 | 1337 | A004p001A007p001C004pU007p | 1384 |
| | | pG007pA004pA007pA004pU007 | | U004pC007pA004pA007pG004pA | |
| | | pU004pC007pU004pU007pG004 | | 007pA004pU007pU004pU007pC0 | |
| | | pA007pA004pG007pU004pU007 | | 04pA007pU004pC007pA004p001 | |
| | | pX2000 | | U004p001U004 | |
| 683 | 3111 | U007p001G004p001A007pA004 | 1338 | A004p001U007p001G004pA007p | 1385 |
| | | pA007pU004pU007pC004pU007 | | A004pC007pU004pU007pC004pA | |
| | | pU004pG007pA004pA007pG004 | | 007pA004pG007pA004pA007pU0 | |
| | | pU007pU004pC007pA004pU007 | | 04pU007pU004pC007pA004p001 | |
| | | pX2000 | | U004p001U004 | |
| 684 | 3208 | A007p001C004p001U007pC004 | 1339 | U004p001A007p001A004pC007p | 1386 |
| | | pc007pU004pG007pA004pU007 | | U004pA007pC004pA007pA004pA | |
| | | pU004pU007pU004pG007pU004 | | 007pA004pU007pC004pA007pG0 | |
| | | pA007pG004pU007pU004pA007 | | 04pG007pA004pG007pU004p001 | |
| | | pX2000 | | U004p001U004 | |
| 685 | 3209 | C007p001U004p001C007pC004 | 1340 | U004p001U007p001A004pA007p | 1387 |
| | | pU007pG004pA007pU004pU007 | | C004pU007pA004pC007pA004pA | |
| | | pU004pU007pG004pU007pA004 | | 007pA004pA007pU004pC007pA0 | |
| | | pG007pU004pU007pA004pA007 | | 04pG007pG004pA007pG004p001 | |
| | | pX2000 | | U004p001U004 | |
| 686 | 3210 | U007p001C004p001C007pU004 | 1341 | A004p001U007p001U004pA007p | 1388 |
| | | pG007pA004pU007pU004pU007 | | A004pC007pU004pA007pC004pA | |
| | | pU004pG007pU004pA007pG004 | | 007pA004pA007pA004pU007pC0 | |
| | | pU007pU004pA007pA004pU007 | | 04pA007pG004pG007pA004p001 | |
| | | pX2000 | | U004p001U004 | |
| 687 | 3254 | C007p001A004p001A007pG004 | 1342 | A004p001C007p001U004pU007p | 1389 |
| | | pA007pA004pG007pU004pA007 | | A004pG007pC004pU007pC004pU | |
| | | pA004pG007pA004pG007pC004 | | 007pU004pA007pC004pU007pU0 | |
| | | pU007pA004pA007pG004pU007 | | 04pC007pU004pU007pG004p001 | |
| | | pX2000 | | U004p001U004 | |
| 688 | 3400 | A007p001C004p001U007pA004 | 1343 | U004p001A007p001A004pC007p | 1390 |
| | | pC007pA004pG007pA004pA007 | | A004pC007pA004pU007pU004pA | |
| | | pU004pA007pA004pU007pG004 | | 007pU004pU007pC004pU007pG0 | |
| | | pU007pG004pU007pU004pA007 | | 04pU007pA004pG007pU004p001 | |
| | | pX2000 | | U004p001U004 | |
| 689 | 3401 | C007p001U004p001A007pC004 | 1344 | U004p001U007p001A004pA007p | 1391 |
| | | pA007pG004pA007pA004pU007 | | C004pA007pC004pA007pU004pU | |
| | | pA004pA007pU004pG007pU004 | | 007pA004pU007pU004pC007pU0 | |
| | | pG007pU004pU007pA004pA007 | | 04pG007pU004pA007pG004p001 | |
| | | pX2000 | | U004p001U004 | |
| 690 | 3483 | A007p001G004p001A007pG004 | 1345 | U004p001U007p001A004pA007p | 1392 |
| | | pA007pG004pG007pC004pC007 | | A004pC007pA004pA007pA004pA | |
| | | pU004pU007pU004pU007pG004 | | 007pG004pG007pC004pC007pU0 | |
| | | pU007pU004pU007pA004pA007 | | 04pC007pU004pC007pU004p001 | |
| | | pX2000 | | U004p001U004 | |
| 691 | 3484 | G007p001A004p001G007pA004 | 1346 | U004p001U007p001U004pA007p | 1393 |
| | | pG007pG004pC007pC004pU007 | | A004pA007pC004pA007pA004pA | |
| | | pU004pU007pU004pG007pU004 | | 007pA004pG007pG004pC007pC0 | |
| | | pU007pU004pA007pA004pA007 | | 04pU007pC004pU007pC004p001 | |
| | | pX2000 | | U004p001U004 | |
| 692 | 3485 | A007p001G004p001A007pG004 | 1347 | A004p001U007p001U004pU007p | 1394 |
| | | pG007pC004pC007pU004pU007 | | A004pA007pA004pC007pA004pA | |
| | | pU004pU007pG004pU007pU004 | | 007pA004pA007pG004pG007pC0 | |
| | | pU007pA004pA007pA004pU007 | | 04pC007pU004pC007pU004p001 | |
| | | pX2000 | | U004p001U004 | |
| 693 | 3533 | G007p001A004p001U007pU004 | 1348 | A004p001G007p001A004pC007p | 1395 |
| | | pG007pG004pC007pU004pA007 | | A004pU007pG004pU007pU004pA | |
| | | pU004pA007pA004pC007pA004 | | 007pU004pA007pG004pC007pC0 | |
| | | pU007pG004pU007pC004pU007 | | 04pA007pA004pU007pC004p001 | |
| | | pX2000 | | U004p001U004 | |
| 694 | 3597 | U007p001A004p001A007pA004 | 1349 | A004p001A007p001G004pA007p | 1396 |
| | | pG007pA004pU007pA004pU007 | | G004pC007pU004pC007pU004pG | |
| | | pC004pA007pG004pA007pG004 | | 007pA004pU007pA004pU007pC0 | |
| | | pc007pU004pC007pU004pU007 | | 04pU007pU004pU007pA004p001 | |
| | | pX2000 | | U004p001U004 | |
| 695 | 3654 | A007p001G004p001A007pC004 | 1350 | A004p0010007p001U004pU007p | 1397 |
| | | pU007pG004pG007pG004pA007 | | C004pU007pA004pA007pG004pG | |
| | | pC004pC007pU004pU007pA004 | | 007pU004pC007pC004pC007pA0 | |
| | | pG007pA004pA007pA004pU007 | | 04pG007pU004pC007pU004p001 | |
| | | pX2000 | | U004p001U004 | |
| 696 | 3826 | G007p001G004p001A007pA004 | 1351 | U004p001U007p001A004pU007p | 1398 |
| | | pG007pU004pG007pU004pU007 | | U004pU007pC004pU007pU004pG | |
| | | pC004pA007pA004pG007pA004 | | 007pA004pA007pC004pA007pC0 | |
| | | pA007pA004pU007pA004pA007 | | 04pU007pU004pC007pC004p001 | |
| | | pX2000 | | U004p001U004 | |
| 697 | 4099 | G007p001C004p001A007pG004 | 1352 | U004p001A007p001A004pG007p | 1399 |
| | | pA007pG004pU007pU004pA007 | | A004pU007pU004pC007pA004pA | |
| | | pU004pU007pG004pA007pA004 | | 007pU004pA007pA004pC007pU0 | |
| | | pU007pC004pU007pU004pA007 | | 04pC007pU004pG007pC004p001 | |
| | | pX2000 | | U004p001U004 | |
| 698 | 4100 | C007p001A004p001G007pA004 | 1353 | U004p001U007p001A004pA007p | 1400 |
| | | pG007pU004pU007pA004pU007 | | G004pA007pU004pU007pC004pA | |
| | | pU004pG007pA004pA007pU004 | | 007pA004pU007pA004pA007pC0 | |
| | | pC007pU004pU007pA004pA007 | | 04pU007pC004pU007pG004p001 | |
| | | pX2000 | | U004p001U004 | |
| 699 | 4102 | G007p001A004p001G007pU004 | 1354 | A004p001A007p001U004pU007p | 1401 |
| | | pU007pA004pU007pU004pG007 | | A004pA007pG004pA007pU004pU | |
| | | pA004pA007pU004pC007pU004 | | 007pC004pA007pA004pU007pA0 | |
| | | pU007pA004pA007pU004pU007 | | 04pA007pC004pU007pC004p001 | |
| | | pX2000 | | U004p001U004 | |
| 700 | 4103 | A007p001G004p001U007pU004 | 1355 | A004p001A007p001A004pU007p | 1402 |
| | | pA007pU004pU007pG004pA007 | | U004pA007pA004pG007pA004pU | |
| | | pA004pU007pC004pU007pU004 | | 007pU004pC007pA004pA007pU0 | |
| | | pA007pA004pU007pU004pU007 | | 04pA007pA004pC007pU004p001 | |
| | | pX2000 | | U004p001U004 | |
| 701 | 4154 | A007p001G004p001A007pA004 | 1356 | A004p001U007p001A004pC007p | 1403 |
| | | pC007pU004pC007pC004pU007 | | A004pA007pA004pA007pU004pA | |
| | | pU004pA007pU004pU007pU004 | | 007pA004pG007pG004pA007pG0 | |
| | | pU007pG004pU007pA004pU007 | | 04pU007pU004pC007pU004p001 | |
| | | pX2000 | | U004p001U004 | |

X2000:

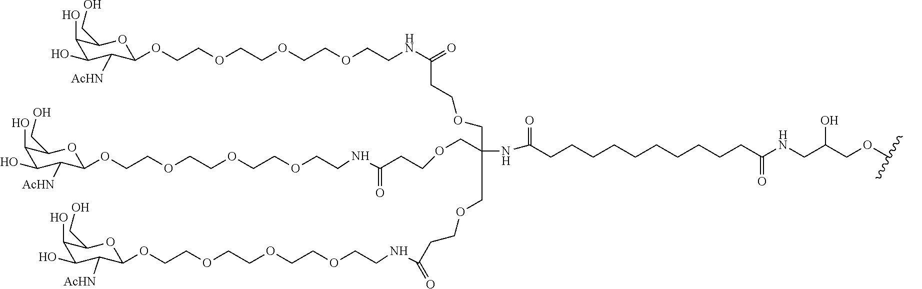

The results of suppression by example HMGCR siRNA agents in Hepa-1-6 cells are shown in Table 9.

TABLE 9

| | | | 80 nM | | 40 nM | | 20 nM | | 10 nM |
|---|---|---|---|---|---|---|---|---|---|
| siRNA | | | Remaining | | Remaining | | Remaining | | Remaining |
| No. | position | IC₅₀ | avr. | SD | avr. | SD | avr. | SD | avr. |
| 655 | 126 | 15.2 | 19.8 | 6.8 | 26.8 | 6.2 | 42.4 | 4.2 | 61.3 |
| 656 | 131 | 11.9 | 19.4 | 6.5 | 24 | 3.6 | 35.6 | 7.5 | 52.3 |
| 657 | 277 | 13.4 | 26.6 | 2.3 | 30.9 | 7.9 | 40 | 10.6 | 53.9 |
| 658 | 295 | 10.4 | 14.9 | 7.2 | 31 | 14.3 | 39.2 | 8.2 | 60.8 |
| 659 | 445 | 9.6 | 15.9 | 1.7 | 21.5 | 1.9 | 33.2 | 6.3 | 52.6 |
| 660 | 730 | 24.4 | 32.3 | 9 | 39.4 | 8.1 | 50.9 | 10.5 | 65.5 |
| 661 | 736 | 7.7 | 20.9 | 4.6 | 26.4 | 1.8 | 35.2 | 3.6 | 44.9 |
| 662 | 739 | 12.2 | 26.2 | 2.9 | 32.1 | 4.4 | 39.9 | 3.8 | 54.3 |
| 663 | 1328 | 7.7 | 24.5 | 15.8 | 29.7 | 11.2 | 42.7 | 4.6 | 40 |
| 664 | 1516 | 34.2 | 40.6 | 3.5 | 45.7 | 7.2 | 57.7 | 6.2 | 67.2 |
| 665 | 1555 | 25.7 | 34.5 | 7.3 | 42.3 | 5.8 | 50.4 | 11.6 | 65.6 |
| 666 | 1558 | 17.8 | 25.8 | 3.4 | 46.2 | 27.4 | 43 | 5.1 | 56.9 |
| 667 | 1586 | 13.4 | 21.8 | 1.9 | 30.7 | 9.9 | 40.3 | 9 | 54.3 |
| 668 | 1996 | 19 | 29.6 | 7.6 | 32.5 | 5.8 | 48.1 | 10.7 | 60.7 |
| 669 | 2169 | 10.4 | 26.2 | 2.5 | 27.7 | 6.4 | 35.9 | 5 | 45.8 |
| 670 | 2269 | 2.7 | 9.1 | 3.4 | 11.4 | 0.8 | 18.9 | 3.7 | 26.9 |
| 671 | 2274 | 9.9 | 25.6 | 1.8 | 28.1 | 2.4 | 34.7 | 4.5 | 45.1 |
| 672 | 2424 | 12.3 | 23.1 | 8.8 | 28.3 | 8.2 | 37 | 7.8 | 52.1 |
| 673 | 2484 | 6.4 | 17.8 | 0.6 | 18.5 | 1.2 | 26.5 | 5.9 | 37.6 |
| 674 | 2575 | 9.4 | 13.5 | 0.9 | 18.7 | 1.4 | 30.5 | 6.3 | 48.5 |
| 675 | 2576 | 6.7 | 18 | 2.8 | 17.8 | 1.1 | 26.7 | 3.4 | 37.6 |
| 676 | 2592 | 7.1 | 19.5 | 1.7 | 21.3 | 1.3 | 30.8 | 1.4 | 40.5 |
| 677 | 2627 | 15 | 25.2 | 6.9 | 28.1 | 6.8 | 39.2 | 10.5 | 56.4 |
| 678 | 2728 | 12.5 | 21.8 | 3.5 | 28.5 | 6 | 38.3 | 4 | 55.9 |
| 679 | 2731 | 10 | 18 | 6.2 | 24.9 | 6 | 33.1 | 8 | 48 |
| 680 | 2737 | 10.7 | 25.5 | 2.1 | 26.8 | 3.4 | 39 | 3.1 | 50.9 |
| 681 | 2937 | 9.3 | 20.2 | 2 | 22.5 | 1.9 | 35.3 | 3.8 | 46.1 |
| 682 | 3108 | 6.5 | 21.3 | 1.2 | 23.8 | 1.7 | 30.3 | 7 | 40.9 |
| 683 | 3111 | 9.7 | 19.1 | 1.9 | 24 | 1.2 | 29.4 | 2.4 | 53.9 |
| 684 | 3208 | 14.2 | 25.4 | 7.6 | 28.7 | 11.1 | 40.6 | 10.9 | 55.8 |
| 685 | 3209 | 6.6 | 20.4 | 1.6 | 20.2 | 1 | 26.2 | 2.1 | 39.8 |
| 686 | 3210 | 12.2 | 27.3 | 4 | 31.9 | 6.7 | 41.5 | 5.9 | 58.3 |
| 687 | 3254 | 5.8 | 20.6 | 9.3 | 16.9 | 4.6 | 38.6 | 7.4 | 42.6 |
| 688 | 3400 | 13 | 33.2 | 5.7 | 31.1 | 4.9 | 37 | 3.9 | 54.2 |
| 689 | 3401 | 17.7 | 36.3 | 7.3 | 37.8 | 7.6 | 43.2 | 13.7 | 55.4 |
| 690 | 3483 | 10.6 | 29 | 4.1 | 31.2 | 4.4 | 36 | 7.9 | 48.2 |
| 691 | 3484 | 7.8 | 25.5 | 5.3 | 27.8 | 2.4 | 32.4 | 4.7 | 37.4 |
| 692 | 3485 | 12.3 | 31.5 | 3.4 | 34.6 | 8.1 | 39.6 | 7.8 | 47.3 |
| 693 | 3533 | 13.6 | 31.7 | 4.8 | 33.9 | 3.4 | 39.6 | 2.4 | 50.2 |
| 694 | 3597 | 19.1 | 40.8 | 4.1 | 37.5 | 4.9 | 45.2 | 11.2 | 56 |
| 695 | 3654 | 9.1 | 28.2 | 4.5 | 27.8 | 1 | 33.8 | 0.5 | 46 |
| 696 | 3826 | 16.4 | 36.2 | 7.6 | 33.2 | 7 | 39.1 | 5.8 | 53.3 |
| 697 | 4099 | 13.4 | 32.1 | 1.9 | 32.2 | 2.2 | 42.5 | 3.6 | 51 |
| 698 | 4100 | 23.9 | 49.7 | 7.3 | 40.8 | 9.5 | 42.5 | 6.4 | 54.3 |
| 699 | 4102 | 17.6 | 39.9 | 2.7 | 38.6 | 6.2 | 41.8 | 8.8 | 56.3 |
| 700 | 4103 | 13.8 | 40.4 | 4.4 | 35.6 | 1.1 | 41.5 | 3.3 | 49.2 |
| 701 | 4154 | 22.7 | 47.4 | 4.4 | 40.1 | 2.7 | 42 | 3.9 | 57.1 |

| | | | | 5 nM | | SD_2.5 nM | | 1.25 nM | |
|---|---|---|---|---|---|---|---|---|---|
| siRNA | | | 10 nM | Remaining | | Remaining | | Remaining | |
| No. | position | IC₅₀ | SD | avr. | SD | avr. | SD | avr. | SD |
| 655 | 126 | 15.2 | 9.1 | 79 | 5.3 | 78.9 | 7.6 | 86.3 | 18.9 |
| 656 | 131 | 11.9 | 14.9 | 70.2 | 8.4 | 75.8 | 15.3 | 91.2 | 13.3 |
| 657 | 277 | 13.4 | 14.8 | 76 | 6.3 | 70.8 | 9.1 | 72.8 | 14.3 |
| 658 | 295 | 10.4 | 19.3 | 57.4 | 16.1 | 79.8 | 37.7 | 68.3 | 10.8 |
| 659 | 445 | 9.6 | 14.2 | 69.1 | 8.6 | 72.5 | 5.3 | 85.2 | 19.9 |
| 660 | 730 | 24.4 | 14 | 81.1 | 8.4 | 81.8 | 8 | 91.2 | 22.7 |
| 661 | 736 | 7.7 | 3.9 | 53.4 | 4.1 | 67.8 | 5.7 | 76.5 | 14.1 |
| 662 | 739 | 12.2 | 11.4 | 63.1 | 5.9 | 71.9 | 8.8 | 80.3 | 13 |
| 663 | 1328 | 7.7 | 8.8 | 51.7 | 13.9 | 66.4 | 15.1 | 69 | 14.8 |
| 664 | 1516 | 34.2 | 4.7 | 76 | 4.7 | 80.5 | 5.5 | 90.3 | 6.1 |
| 665 | 1555 | 25.7 | 18.2 | 78 | 10.3 | 78.1 | 13 | 87.9 | 18.9 |
| 666 | 1558 | 17.8 | 9.3 | 66.8 | 8.5 | 77.7 | 10 | 86.9 | 20.7 |
| 667 | 1586 | 13.4 | 12.3 | 70.4 | 12.5 | 83 | 19 | 80.3 | 21 |
| 668 | 1996 | 19 | 16.3 | 77.3 | 15.9 | 81.5 | 10.7 | 87.3 | 18.8 |
| 669 | 2169 | 10.4 | 7.9 | 61.1 | 5.6 | 68.6 | 7 | 84.7 | 19.5 |
| 670 | 2269 | 2.7 | 5.2 | 57.2 | 6 | 61.4 | 10.6 | 57.6 | 7.2 |
| 671 | 2274 | 9.9 | 6.3 | 60.5 | 6.1 | 74.5 | 9.1 | 82.3 | 10.5 |
| 672 | 2424 | 12.3 | 16.3 | 67.5 | 7.4 | 76.4 | 6.8 | 87.7 | 19.2 |
| 673 | 2484 | 6.4 | 10.5 | 59 | 6.1 | 68.5 | 17.5 | 70 | 7.1 |
| 674 | 2575 | 9.4 | 8.8 | 74.6 | 9.4 | 76.9 | 8 | 79.7 | 8.2 |
| 675 | 2576 | 6.7 | 4.9 | 56.9 | 3.3 | 66.8 | 11.5 | 82.9 | 14.1 |
| 676 | 2592 | 7.1 | 2.4 | 54.9 | 1.9 | 65.2 | 9.1 | 83.4 | 18 |
| 677 | 2627 | 15 | 14.4 | 77.9 | 13.7 | 80.8 | 13.5 | 87.1 | 16.9 |
| 678 | 2728 | 12.5 | 12 | 67.7 | 14.1 | 75.9 | 6.6 | 88.1 | 18.5 |
| 679 | 2731 | 10 | 8.3 | 66.3 | 5.4 | 75.4 | 4.7 | 80.2 | 20 |
| 680 | 2737 | 10.7 | 12.1 | 66.9 | 7.8 | 73.6 | 11.9 | 73.8 | 11.2 |
| 681 | 2937 | 9.3 | 7.6 | 66.4 | 4.4 | 72.4 | 13.8 | 77.8 | 6.7 |
| 682 | 3108 | 6.5 | 6.8 | 54.6 | 10.9 | 61.4 | 2.2 | 78.1 | 6.7 |
| 683 | 3111 | 9.7 | 9.8 | 67.8 | 6.1 | 73.7 | 4.7 | 79.4 | 7 |
| 684 | 3208 | 14.2 | 24.9 | 66.6 | 12.2 | 81.9 | 14.1 | 89.1 | 28.2 |
| 685 | 3209 | 6.6 | 5.5 | 57 | 5.5 | 67 | 6 | 71.7 | 2.5 |
| 686 | 3210 | 12.2 | 10.7 | 64.8 | 7.1 | 69.9 | 7.9 | 71.9 | 9.1 |
| 687 | 3254 | 5.8 | 10.6 | 49.1 | 13 | 60.7 | 13.9 | 74.1 | 17 |
| 688 | 3400 | 13 | 8.4 | 66.5 | 4.7 | 75.7 | 14.9 | 75.7 | 16.6 |
| 689 | 3401 | 17.7 | 15.6 | 62.1 | 5.6 | 78.1 | 14.9 | 83.7 | 23.3 |
| 690 | 3483 | 10.6 | 6.9 | 63.5 | 2 | 70.4 | 11.5 | 81.4 | 11.3 |
| 691 | 3484 | 7.8 | 3.4 | 52.4 | 5.6 | 73.4 | 9.5 | 76 | 11.9 |
| 692 | 3485 | 12.3 | 6.2 | 65.1 | 5.2 | 70.4 | 5.9 | 77.7 | 14.3 |
| 693 | 3533 | 13.6 | 7.8 | 67.6 | 2.9 | 74.5 | 7.8 | 81 | 11.9 |
| 694 | 3597 | 19.1 | 11.9 | 66 | 6.2 | 74.7 | 15.1 | 75.9 | 5.7 |
| 695 | 3654 | 9.1 | 2 | 58.2 | 1.8 | 68.1 | 6.3 | 80.2 | 17.8 |
| 696 | 3826 | 16.4 | 8.5 | 67 | 6.3 | 79.6 | 17.4 | 98.7 | 29.6 |
| 697 | 4099 | 13.4 | 4.7 | 64.5 | 4.4 | 77 | 17.3 | 73.3 | 6.7 |
| 698 | 4100 | 23.9 | 12.1 | 64 | 6.9 | 82.1 | 8.9 | 98.2 | 32.6 |
| 699 | 4102 | 17.6 | 12.8 | 66.6 | 9 | 73.9 | 18.2 | 71.3 | 6 |
| 700 | 4103 | 13.8 | 6 | 57.9 | 4.2 | 70.9 | 9.6 | 74.7 | 15.1 |
| 701 | 4154 | 22.7 | 10.7 | 65.8 | 7.6 | 73.9 | 7.1 | 87.9 | 16.2 |

The siRNA sequences were selected using a bioinformatics algorithm to identify antisense strands that are complementary to the human and cynomolgus monkey HMGCR transcripts. In the primary screen, human primary hepatocytes (PHH) in 96-well plates were treated with 10 μM or 0.5 μM GalNAc-conjugated siRNA to facilitate free uptake via the asialoglycoprotein receptor (ASPGR). Forty-eight hours post-treatment, percent remaining HMGCR mRNA, relative to mock-treated (PBS) cells, was measured by a branched DNA assay. The 48 most active siRNAs from this primary screen progressed to full dose response curve analysis using a sensitive reporter assay. To this end, a plasmid expressing HMGCR mRNA upstream of a luciferase reporter cassette was transfected in Hepa 1-6 cells (a murine hepatoma cell line) together with the siRNAs. Percent remaining luciferase activity was then measured 24 hours post-transfection relative to mock-treated cells.

The active sequences were carried forward for lead optimization, where in the first phase 5 different chemical formats with specific positioning of 2′-Fluoro (2′-F), 2′O-Methyl (2′-OMe) and/or 2′-Deoxy along the antisense and sense strands were tested in each of the 12 siRNAs using a reporter DRC readout. Phosphorothioate (PS) linkages were kept constant, with 6 in total. Chemical characteristics that improved or did not diminish in vitro potency included increased 2′-OMe content and reduced 2′-F content. In this optimized 2′-F/2′OMe format, all 12 sequences were next characterized for their target specificity by transcriptome-wide analysis using RNA sequencing (RNA-Seq).

Briefly, siRNAs were transfected into Hep3B cells (a human hepatoma cell line) at a concentration of 20 nM using RNAiMax, and differential gene expression relative to mock-treated (PBS+RNAiMax) cells was calculated 24 hours post-treatment. The selected sequences were shown to be highly specific, with no gene other than its target, HMGCR, showing significant regulation.

The additional optimization led to siRNA sequences of Table 4 or Compounds in Table 5. For example, Compound 1 exhibited an in vitro potency of IC₅₀=3.4 μM. In human liver cell lines, Compound 1 significantly reduces HMGCR mRNA and protein concentrations, while it increases LDLR mRNA and protein levels.

### Example 9: Pharmacology of HMGCR siRNA in Animal Models

Increased hepatic LDLR density and, in turn, enhanced clearance of LDL from the circulation, is observed with statin treatment; thus, it is anticipated that Compound 1 will similarly reduce plasma LDL levels in humans.

9.1 In Vivo in Chimeric Mice with Humanized Liver

Consistent with this hypothesis, Compound 1 reduced plasma total cholesterol concentrations by >50% through day 34 in a chimeric mouse model with humanized liver.

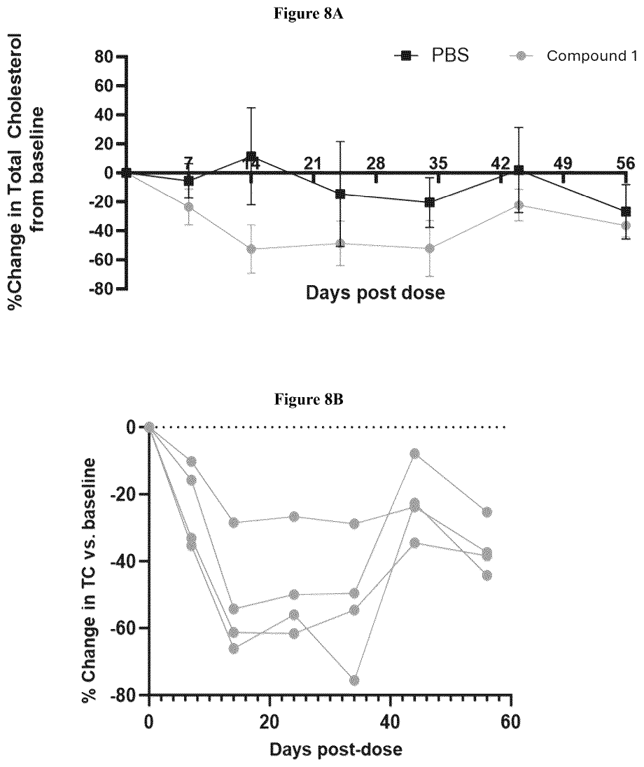

Compound 1 was evaluated in chimeric mice engrafted with primary human hepatocytes. Similar to humans, the majority of plasma cholesterol in this model is carried in LDL and not in HDL as is the case for mice. Male mice were administered a single subcutaneous dose of Compound 1 at 3 mg/kg or PBS (n=4 per group), and blood samples were collected through day 56 for the measurement of total plasma cholesterol. On day 56, livers were harvested for the determination of HMGCR mRNA abundance and RISC-loading. As shown in FIGS. 8A-8B a single 3 mg/kg dose of Compound 1 reduced plasma cholesterol versus baseline by >50% through day 34. Plasma total cholesterol levels were reduced in all mice treated with Compound 1, with 3 of 4 mice having maximum reductions >50%. Liver HMGCR mRNA levels 56 days post-dose did not differ between the PBS and Compound 1 groups, with mean values of 4.0+1.7 and 4.1+1.8, respectively, relative to the house-keeping gene (TATA-binding protein). Consistent with the lack of mRNA reduction seen at day 56 post—dose, RISC-loading for Compound 1 was very low (0.11+0.06 ng siRNA/g tissue).

### 9.2 Pharmacology of Compound 1 in Cynomolgus Monkeys

The pharmacodynamic effects of a single 10 mg/kg subcutaneous dose of Compound 1 were evaluated in male cynomolgus monkeys (n=4 per group). Liver biopsy samples were obtained 21 days post-dose to enable the measurement of liver HMGCR mRNA abundance, total liver siRNA levels, and incorporation of the guide strand of Compound 1 into RISC. Compound 1 reduced liver HMGCR mRNA abundance by a mean of 63% (P<0.001) when compared to vehicle (sterile saline). The magnitude of mRNA reduction was similar among the 4 animals, ranging from −55% to −74%. Mean liver siRNA and RISC-loading values 21 days post-dose were 127100±90529 and 17.4±12.1 ng siRNA/g tissue, respectively. These results show that a single 10 mg/kg dose of Compound 1 elicits a significant pharmacodynamic response in cynomolgus monkeys.

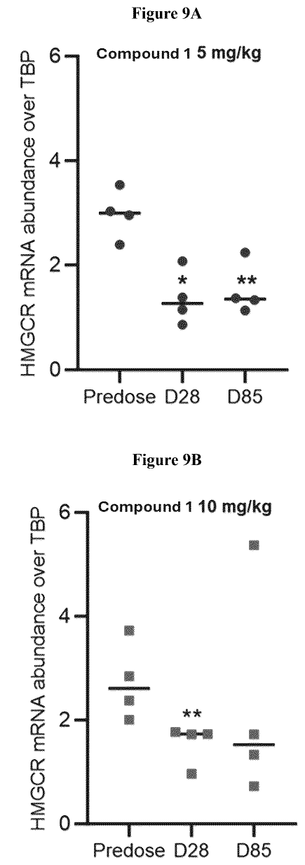

Compound 1's impact on the magnitude and durability of liver HMGCR mRNA knockdown was assessed in male cynomolgus monkeys. On days 1 and 35, animals received Compound 1 at 5 or 10 mg/kg (n=4 per group) via subcutaneous injection. Liver biopsy samples were obtained at pre-dose (day −12) and two post-dose timepoints (days 28 and 85 after the first dose) to enable the measurement of liver HMGCR mRNA abundance, total liver siRNA levels, and incorporation of the guide strand of Compound 1 into RISC. In animals administered Compound 1 at 5 mg/kg, hepatic HMGCR mRNA abundance at day 28 was reduced by 54% (P<0.0003) versus pre-dose (FIGS. 9A-9B), with a similar magnitude of target knockdown (KD) observed on day 85 (−49%, P<0.05), 50 days after administration of a second Compound 1 dose. As shown in FIGS. 9A-9B, Compound 1 did not reduce liver HMGCR mRNA abundance in a dose-dependent manner, with animals in the 10 mg/kg group showing relatively inferior HMGCR KD versus pre-dose compared to animals in the 5 mg/kg group at both day 28 (−43%, P<0.05) and 85 (−16%, P=NS).

In contrast to the HMGCR mRNA results, where dose-dependency was not observed for animals administered Compound 1, dose-dependency was seen for liver total siRNA concentrations and RISC-loading. Mean liver siRNA concentrations (ng siRNA/g tissue) for the Compound 1 5 and 10 mg/kg groups were 10300±4600 and 15900±3100, respectively, on day 28 post-dose, with corresponding values of 16000±5900 and 43700±24000 on day 85 post initial dose. Mean RISC-loading values (ng siRNA/g tissue) at day 28 post-dose were 3.4±1.4 and 10.3±2.2 for the Compound 1 5 and 10 mg/kg groups, respectively, with corresponding values of 13.9±9.3 and 27.3±10.9 for day 85 post-dose. Greater RISC-loading was observed after two doses (day 85) versus a single dose (day 28) of siRNA had been given.

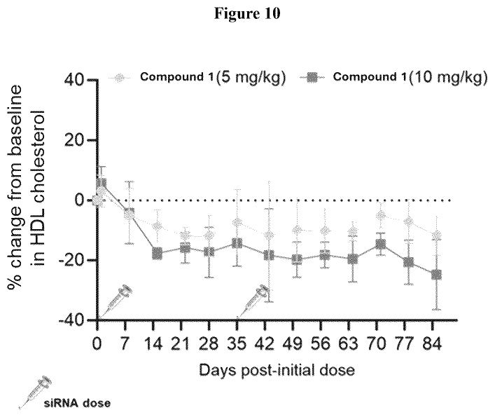

As expected, Compound 1 did not significantly alter serum cholesterol levels in this study (data not shown); however, it should be noted that the magnitude of HDL cholesterol reduction observed in the Compound 1 10 mg/kg group (−25%; FIG. 10) is on par with that of −28% reported for male cynomolgus monkeys treated with high dose atorvastatin for 85 days, In summary, Compound 1 at 5 or 10 mg/kg reduced mean hepatic HMGCR mRNA abundance versus pre-dose at both day 28 and 85; however, only the Compound 1, 5 mg/kg group showed statistically significant reductions in target knockdown at both timepoints.

### Example 10: In Vitro Comparison

### 10.1 Methods

Sample siRNAs tested in Example 10 are listed in Table 10. Specific codes in the nucleotide sequences are indicated in above, e.g., Tables A and A-1.

TABLE 10

| | position | | SEQ | | SEQ |
|---|---|---|---|---|---|
| | in | Sense Strand | ID | Antisense strand | ID |
| siRNA | mRNA | Sense Strand (modified) | NO | Antisense Strand (modified) | NO |
| S1 | 127 | GUUGUCAAGACUUUUUCGAAU | 1404 | AUUCGAAAAAGUCUUGACAACAU | 1419 |
| | | G004p001U004p001U004pG004 | 1405 | A004p001U007p001U004pC004p | 1420 |
| | | pU004pC004pA007pA004pG007 | | G004pA007pA004pA007pA007pA | |
| | | pA007pC007pU004pU004pU004 | | 004pG004pU004pC004pU007pU0 | |
| | | pU004pU004pC004pG004pA004 | | 04pG007pA004pC004pA004pA00 | |
| | | pA004pU004pX2000 | | 4pC004p001A004p001U004 | |
| S2 | 127 | GUUGUCAAGACUUUUUCGAAA | 1406 | UUUCGAAAAAGUCUUGACAAC | 1421 |
| | | G004p001U004p001U004pG004 | 1407 | U004p001U007p001U004pC004p | 1422 |
| | | pU004pC004pA004pA004pG007 | | G004pA004pA007pA004pA004pA | |
| | | pA004pC007pU004pU007pU004 | | 004pG004pU007pC004pU007pU0 | |
| | | pU004pU004pC004pG004pA004 | | 04pG007pA004pC004pA004p001 | |
| | | pA004p001A004pX2000 | | A004p001C004 | |
| S3 | 129 | UGUCAAGACUUUUUCGAAUGA | 1408 | UCAUUCGAAAAAGUCUUGACA | 1423 |
| | | U004p001G004p001U004pC004 | 1409 | U004p001C007p001A004pU004p | 1424 |
| | | pA004pA004pG004pA004pC007 | | U004pC004pG007pA004pA004pA | |
| | | pU004pU007pU004pU007pU004 | | 004pA004pA007pG004pU007pC0 | |
| | | pC004pG004pA004pA004pU004 | | 04pU007pU004pG004pA004p001 | |
| | | pG004p001A004pX2000 | | C004p001A004 | |
| S4 | 124 | AAUGUUGUCAAGACUUUUUCA | 1410 | UGAAAAAGUCUUGACAACAUU | 1425 |
| | | A004p001A004p001U004pG004 | 1411 | U004p001G007p001A004pA004p | 1426 |
| | | pU004pU004pG004pU004pC007 | | A004pA004pA007pG004pU004pC | |
| | | pA004pA007pG004pA007pC004 | | 004pU004pU007pG004pA007pC0 | |
| | | pU004pU004pU004pU004pU004 | | 04pA007pA004pC004pA004p001 | |
| | | p001C004p001A004pX2000 | | U004p001U004 | |
| S5 | 125 | AUGUUGUCAAGACUUUUUCGA | 1412 | UCGAAAAAGUCUUGACAACAU | 1427 |
| | | A004p001U004p001G004pU004 | 1413 | U004p001C007p001G004pA004p | 1428 |
| | | pU004pG004pU004pC004pA007 | | A004pA004pA007pA004pG004pU | |
| | | pA004pG007pA004pC007pU004 | | 004pC004pU007pU004pG007pA0 | |
| | | pU004pU004pU004pU004pC004 | | 04pC007pA004pA004pC004p001 | |
| | | p001G004p001A004pX2000 | | A004p001U004 | |
| S6 | 126 | UGUUGUCAAGACUUUUUCGAA | 1414 | UUCGAAAAAGUCUUGACAACA | 1429 |
| | | U004p001G004p001U004pU004 | 1415 | U004p001U007p001C004pG004p | 1430 |
| | | pG004pU004pC004pA004pA007 | | A004pA004pA007pA004pA004pG | |
| | | pG004pA007pC004pU007pU004 | | 004pU004pC007pU004pU007pG0 | |
| | | pU004pU004pU004pC004pG004 | | 04pA007pC004pA004pA004p001 | |
| | | p001A004p001A004pX2000 | | C004p001A004 | |
| S7 | 123 | GAAUGUUGUCAAGACUUUUUA | 1416 | UAAAAAGUCUUGACAACAUUC | 1431 |
| | | G004p001A004p001A004pU004 | 1417 | U004p001A007p001A004pA004p | 1432 |
| | | pG004pU004pU004pG004pU007 | | A004pA004pG007pU004pC004pU | |
| | | pC004pA007pA004pG007pA004 | | 004pU004pG007pA004pC007pA0 | |
| | | pC004pU004pU004pU004pU004 | | 04pA007pC004pA004pU004p001 | |
| | | p001U004p001A004pX2000 | | U004p001C004 | |
| S8 | 126 | UGUUGUCAAGACUUUUUCGAA | 3 | UUCGAAAAAGUCUUGACAACAUU | 408 |
| | | T005p001G005p001U004pU004 | 1418 | X033U1027p001U007p001C004p | 1433 |
| | | pG004pU004pC007pA004pA007 | | G004pA004pA007pA004pA004pA | |
| | | pG007pA007pC004pU004pU004 | | 004pG004pU004pC004pU004pU0 | |
| | | pU004pU004pU004pC004pG004 | | 07pG004pA007pC004pA004pA00 | |
| | | pA005pA005px2000 | | 4pC004pA004p001U004p001U00 | |
| | | | | 4 | |
| S9 | 126 | UGUUGUCAAGACUUUUUCGAA | 3 | UUCGAAAAAGUCUUGACAACAUU | 408 |
| | | T005p001G005p001U004pU004 | 1302 | X033U1027p001U007p001C004p | 1306 |
| | | pG004pU004pC007pA004pA007 | | G004pA004pA007pA004pA004pA | |
| | | pG007pA007pC004pU004pU004 | | 004pG004pU004pC004pU004pU0 | |
| | | pU004pU004pU004pC004pG004 | | 07pG004pA007pC004pA004pA00 | |
| | | pA005pA005px1085 | | 4pC004pA004p001U004p001U00 | |
| | | | | 4 | |

### 10.1.1 Cell Culture

The human hepatoma Hep3B cell line was obtained from ATCC (cat #HB-8064). Hep3B cells are cultured in EMEM with L-Glut (ATCC 30-2003)+10% FBS medium (heat inactivated). At confluence, the cells were detached with a 2- to 5-min incubation at 37° C. in a Trypsin/EDTA solution (Sigma, cat. T3924) and passaged at a split ratio of 1:4. The medium was renewed every three days. Hepa 1-6 cells (ATCC, cat #CRL-1830), a murine hepatoma cell line, were cultured in DMEM (Gibco Cat.10313021)+1% L-Glut (Gibco, Cat.25030081)+10% FBS (Gibco, cat #A5256701) medium at 37° C. with 5% CO2. At confluence, the cells were detached with a 2-5 min incubation with 0.25% Trypsin/EDTA (Gibco, cat #25200056) and passaged at a split ratio of 1:3.

### 10.1.2 Luciferase Reporter Assay

Hepa-1-6 cells were seeded at 15,000 cells/well in a 96-well plate. At the same time, 100ng/well of reporter plasmid contain human HMGCR mRNA combined with an siRNA at a given concentration were transfected with lipofecamine (Invitrogen, Lipofectamine 2000, cat #11668500). 24 hrs post-transfection, cells were lysed, and luciferase activity was measured based on manufacturer protocol (Promega, DualGlow, cat #E2910). Luciferase signal got normalized to renilla signal to calculate % remaining luciferase. Plasmid herein used were either TR030, which contains HMGCR mRNA form 1-2452 (NM_000859) or TR029 plasmid containing the targeting site for siRNA Sample 9 and others with each a flanking region of 50 bases up- and downstream.

10.1.3 qPCR

Hep3B cells were seeded at 30,000 cells/well in a 96-well plate. Cells were treated simultaneously with siRNA in an 8-step dose response starting at 40 nM in a 1:6 dilution factor.

24 hrs post-transfection cells were lysed using Fast Lane kit (Qiagen, Cat: 204845) as qPCR was performed as described by manufacturer protocol using HMGCR primer (Thermofisher, Cat: Hs00168352_m1) and for housekeeping PGK1 (Thermofisher, Cat: Hs99999906_m1) to calculate fold change by delta delta CT method.

### 10.2 Results

### 10.2.1 Dose Response Curve in Hep3B Cells

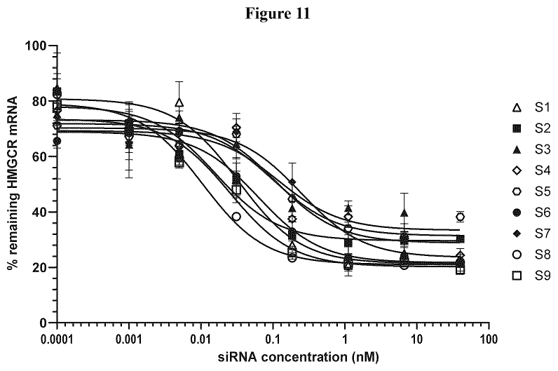

A set of nine siRNAs (S1-S9) was tested for potency in an immortalized hepatoma cell line (Hep3) using lipid-mediated transfection as these cells do not express the receptor ASGPR that would enable GalNAc-mediated uptake. In order to characterize IC₅₀, a dose response for all nine siRNA was done and HMGCR mRNA levels were determined by RT-qPCR 24 hrs post-transfection (FIG. 11). S1-S8 all carry the same GalNAc. Among them, S8 demonstrated the lowest IC₅₀ (0.00835), indicating that S8 is most effective in their potency to lower HMGCR mRNA levels. This is consistent with area under the curve (AUC) from the dose response curves (DRC) S8=0.2013. At the concentration of 0.18 nM, where S8 still achieves maximum inhibition, the delta in terms of % remaining HMGCR mRNA levels is most pronounced (Table 11). Maximum inhibition values as displayed in Table 11 are irrespective of concentrations.

TABLE 11
IC₅₀, AUC and max. inhibition values
from Hep3B does response curve

| | | | max. | % remaining mRNA |
|---|---|---|---|---|
| Sample ID | IC₅₀ (nM) | AUC | inhib. [%] | (0.18 nM) |
| S1 | 0.031644 | 0.233774 | 79.216 | 27.952 |
| S2 | 0.009125 | 0.284179 | 71.235 | 31.618 |
| S3 | 3.959853 | 0.290596 | 78.713 | 41.421 |
| S4 | 0.101888 | 0.316848 | 75.652 | 37.482 |
| S5 | 0.167659 | 0.349543 | 69.033 | 44.624 |
| S6 | 0.058659 | 0.229060 | 78.678 | 32.941 |
| S7 | 0.217291 | 0.261891 | 77.241 | 50.779 |
| S8 | 0.00835 | 0.201322 | 79.388 | 23.382 |
| S9 | 0.016668 | 0.194750 | 80.816 | 25.292 |

### 10.2.2 Dose Response Curve Using a Reporter Assay

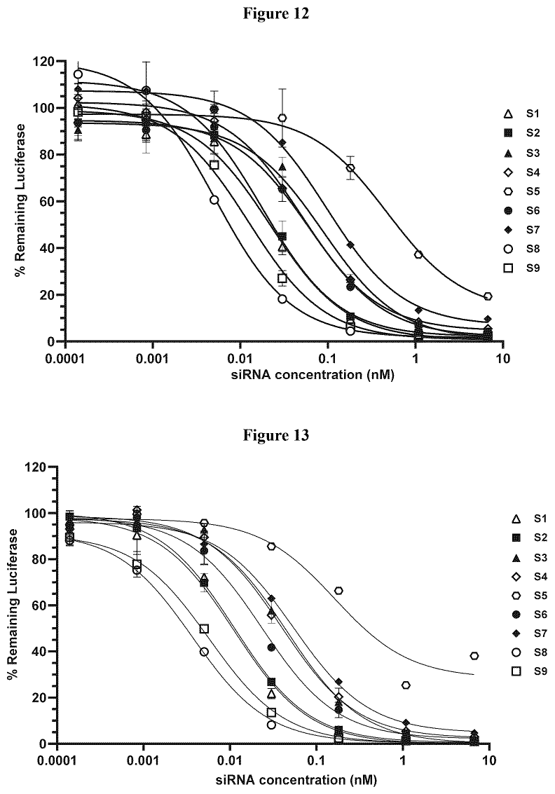

To confirm the results as observed in Hep3B, where S8 und S9 were the most active siRNAs, an independent assay was established to assess activity of siRNAs S1-S9. To this end, HMGCR mRNA from position 1-2452 (NM_000859, e.g., SEQ ID NO: 811 (human HMGCR isoform, transcript variant 1, mRNA (GenBank: NM_000859.3)) was cloned downstream of a luciferase reporter cassette in a plasmid. Co-transfection of this plasmid with siRNA S1-59 in a dose-dependent manner confirmed the observations in Hep3B cells, where S8 and S9 were most active (FIG. 12). S8 that has the matching GalNAc to S1-57, rose to the top again with an IC₅₀ value=0.005320 nM (Table 12). Although maximum inhibition for all tested siRNAs was very comparable, S8 and S9 still achieved maximum luciferase repression at 1.086 nM compared to S1 to S7 (Table 12).

TABLE 12
IC₅₀, AUC and max. inhibition values
from reporter assay in Hepa-1-6 cells

| Sample | | | max. | % remaining luciferase |
|---|---|---|---|---|
| ID | IC₅₀ (nM) | AUC | inhib. [%] | (1.086 nM) |
| S1 | 0.022436 | 0.034901052 | 97.460 | 3.018 |
| S2 | 0.015888 | 0.027642447 | 97.087 | 3.729 |
| S3 | 0.085223 | 0.068634754 | 96.367 | 7.617 |
| S4 | 0.050911 | 0.080424185 | 94.544 | 8.330 |
| S5 | 0.424173 | 0.288725907 | 80.662 | 37.192 |
| S6 | 0.059638 | 0.068209618 | 95.783 | 6.874 |
| S7 | 0.096148 | 0.130199048 | 90.366 | 13.404 |
| S8 | 0.005320 | 0.032591102 | 97.381 | 2.653 |
| S9 | 0.012704 | 0.030741005 | 97.543 | 2.545 |

This result of plasmid TR030 could be further confirmed in another reporter plasmid (TR029), that only contains the binding sites, such as the one from S8 and S9 with a flanking region of 50 nucleotides upstream and downstream to also enable binding of S1 to S7 siRNAs. As previously described for TR030, also TR029 was co-transfected with respective siRNA in Hepa-1-6 cells. The overall ranking of the experiment in TR029 is the same as previously observed for TR030. S8 rose once again to the top with an IC₅₀ of 0.003896 nM (Table 13). Furthermore, differences in potency could be observed at 0.181 nM, where S8 and S9 still achieve maximum repression of luciferase signal, but not siRNAs S1 to S7 (Table 13).

TABLE 13
IC₅₀, AUC and max. inhibition values
from reporter assay in Hepa-1-6 cells

| Sample | | | max. inhib. | % remaining luciferase |
|---|---|---|---|---|
| ID | IC₅₀ (nM) | AUC | [%] | (0.181 nM) |
| S1 | 0.011523 | 0.02273 | 98.958 | 5.931 |
| S2 | 0.011253 | 0.018336 | 98.907 | 6.014 |
| S3 | 0.043469 | 0.044469 | 98.052 | 18.181 |
| S4 | 0.039048 | 0.05676 | 96.908 | 20.323 |
| S5 | 0.184495 | 0.334289 | 74.679 | 66.325 |
| S6 | 0.023317 | 0.046287 | 98.399 | 14.718 |
| S7 | 0.054763 | 0.079133 | 95.300 | 26.864 |
| S8 | 0.003896 | 0.011732 | 98.944 | 1.958 |
| S9 | 0.005801 | 0.009271 | 98.997 | 2.406 |

### Example 11: Introduction of TNA Clamps on Sense Strand of siRNA

### 11.1 Methods

Sample siRNAs tested in Example 11 are listed in Table 14 and prepared as described above.

TABLE 14

| | position | | SEQ | | SEQ |
|---|---|---|---|---|---|
| | in | | ID | | ID |
| siRNA | mRNA | Sense Strand | NO: | Antisense Strand | NO: |
| No1 | 2576 | T005p001T005p001G004pC00 | 1483 | X033U1027p001U007p001G004p | 1299 |
| | | 4pA004pG004pA007pU004pG0 | | A004pA004pC007pA004pC004pC | |
| | | 07pC007pU007pA004pG004pG | | 004pU004pA004pG004pC004pA0 | |
| | | 004pU004pG004pU004pU004p | | 07pU004pC007pU004pG004pC00 | |
| | | C004pA005pA005pX2000 | | 4pA004pA004p001A004p001C00 | |
| | | | | 4 | |
| No2 | 2576 | T005p001T005p001G004pC00 | 1484 | X033U1027p001U007p001G004p | 1299 |
| | | 4pA004pG004pA007pU004pG0 | | A004pA004pC007pA004pC004pC | |
| | | 07pC007pU007pA004pG004pG | | 004pU004pA004pG004pC004pA0 | |
| | | 004pU004pG004pU004pU004p | | 07pU004pC007pU004pG004pC00 | |
| | | C004p001A005p001A005px20 | | 4pA004pA004p001A004p001C00 | |
| | | 00 | | 4 | |
| No3 | 2576 | U042p001U042p001G004pC00 | 1485 | X033U1027p001U007p001G004p | 1299 |
| | | 4pA004pG004pA007pU004pG0 | | A004pA004pC007pA004pC004pC | |
| | | 07pC007pU007pA004pG004pG | | 004pU004pA004pG004pC004pA0 | |
| | | 004pU004pG004pU004pU004p | | 07pU004pC007pU004pG004pC00 | |
| | | C004pA042pA042pX2000 | | 4pA004pA004p001A004p001C00 | |
| | | | | 4 | |
| No4 | 2576 | U042p001U042p001G004pC00 | 1486 | X033U1027p001U007p001G004p | 1299 |
| | | 4pA004pG004pA007pU004pG0 | | A004pA004pC007pA004pC004pC | |
| | | 07pC007pU007pA004pG004pG | | 004pU004pA004pG004pC004pA0 | |
| | | 004pU004pG004pU004pU004p | | 07pU004pC007pU004pG004pC00 | |
| | | C004pA042p001A042p001X20 | | 4pA004pA004p001A004p001C00 | |
| | | 00 | | 4 | |
| No5 | 2576 | U004p001U004p001G004pC00 | 1487 | X033U1027p001U007p001G004p | 1299 |
| | | 4pA004pG004pA007pU004pG0 | | A004pA004pC007pA004pC004pC | |
| | | 07pC007pU007pA004pG004pG | | 004pU004pA004pG004pC004pA0 | |
| | | 004pU004pG004pU004pU004p | | 07pU004pC007pU004pG004pC00 | |
| | | C004pA004pA004pX2000 | | 4pA004pA004p001A004p001C00 | |
| | | | | 4 | |

### 11.2 Animals and Experimental Design

### 11.2.1 Maintenance Conditions

All mice were received at 10 weeks of age and acclimated for at least 3 days prior to experimentation. Animals were maintained on a 12 hour light/dark cycle at 70° F. and 50% humidity, provided water and food ad libitum.

### 11.2.2 Statement on Animal Welfare

Studies described were performed according to an institutional Animal Care and Use Committee (ACUC) approved protocol. All mice were maintained in our pathogen-free and viral-free institutional housing facilities and were sacrificed by CO2 asphyxiation, and confirmed by thoracotomy, as approved by the panel on Euthanasia at the American Veterinary Association, and in the above referenced ACUC protocol.

### 11.2.3 Study Protocol

Male, 10-week-old C57BL/6J mice (Jackson Laboratories, Bar Harbor, ME) were fed a western diet (Research Diets, 12079Bi) for 21 days prior to initiation of the study. On day 0 of the study, body weight data was collected for all animals. Mice were mechanically restrained, and 25 μL of baseline blood was collected via tail snip into EDTA-K2 treated microvette tubes (Sarstedt AG, Sarstedt, Germany) stored on ice. Blood samples were centrifuged at 16,000 g, 4° C. for 10 minutes, with resulting plasma aliquoted and frozen at −80° C. for subsequent measurement of total cholesterol levels.

Each mouse was then given a single subcutaneous (SC) dose of either sterile PBS (10 mL/kg) or siRNA (3 mg/kg) formulated in PBS. Each group of three or four mice received PBS control or siRNAs (No1, No2, No3, No4, and No5). Each week, for 6 weeks, animals from each of the PBS and treatment groups receiving the siRNAs (No1, No2, No3, No4, and No5) were euthanized, while, and each week, 25 μL of blood was collected via tail snip for all mice remaining in the study, for measuring of total cholesterol in the resulting plasma. Each week, designated mice were euthanized via CO₂ asphyxiation and terminal blood samples were collected via cardiac puncture using a 1 mL syringe and 25-gauge needle. After removing the needle, blood was ejected from the syringe into an EDTA-K2 treated tube (Sarstedt AG, Sarstedt, Germany) stored on ice and processed as described above with plasma being assayed for total cholesterol levels. The abdomen was then opened, the left lobe of liver excised and (4) 50-75 mg pieces of liver tissue were placed into individual 2 mL Eppendorf tubes, before being frozen on dry ice and stored at −80° C. until analysis.

11.2.4 Determination of Liver HMGCR mRNA Abundance by RT-PCR

Liver RNA extraction was performed using RNeasy lipid mini kit protocol (Qiagen, Germany). Briefly, a 50 mg piece of frozen liver was lysed and homogenized using a Tissue Lyser II (Qiagen, Germany) with one stainless steel bead and Qiazol lysis reagent (Qiagen, Germany). Next, chloroform was added, and phases were separated. RNA was bound, washed, and eluted from RNEasy mini spin columns. The optional on column DNase digestion was performed using the RNase free DNase set (Qiagen, Germany). A total of 40 μL of RNA was eluted from each sample. RNA samples were quantified using NanoDrop 2000 (ThermoFisher, Waltham MA). Quantified RNA samples were diluted and then reverse transcribed using SuperScript Vilo Master mix (ThermoFisher, Waltham MA). Taqman qPCR was performed with the cDNA samples using Taqman gene expression assays Rn00565598_m1 and Rn01455646_m1 (ThermoFisher, Waltham MA).

11.2.5 Incorporation of Guide Strand into Liver RNA-Silencing Complex

### Tissue Lysis:

Each frozen tissue piece provided in screw cap cryo tube was transferred to a 2 mL round bottom microcentrifuge tube that was pre-chilled on dry ice. One dry ice pre-chilled 5 mm stainless steel bead (Qiagen, Germany) was added to the tube containing the frozen tissue. In the cold room, sample tubes were quickly removed from dry ice and added to each tube 1 mL of ice-cold Lysis buffer (50 mM Tris-HCl pH 7.5, 150 mM NaCl, 2 mM EDTA, 0.5% Triton X-100, 1 mM PMSF, 1×EDTA-free protease inhibitor cocktail). Immediately, tissue was lysed with TissueLyser LT (Qiagen, Germany) for 5 min at 50 Hz in cold room. Lysate was then cleared at 20000×g, 10 min, 4° C., and the soluble lysate supernatants were kept on ice. The protein concentration of the soluble lysate for each sample was determined using BCA assay (ThermoFisher, Waltham MA) according to manufacturer's protocol.

### Argonaute 2 Immunoprecipitation (IP):

Dynabead Protein G (ThermoFisher, Waltham MA) was washed with Wash buffer (50 mM Tris-HCl pH 7.5, 150 mM NaCl, 2 mM EDTA, 0.5% Triton X-100) prior to use for IP, and 50 μL of bead slurry was used per sample. Mouse argonaute2 (Ago2) antibody (FUJIFILM Wako Chemicals, Richmond VA) was pre-bound to beads in wash buffer at 4° C., for 2 h on rotating mixer (200 ng antibody used per 50 μL bead slurry). After incubation, the Ago2 antibody-bound beads were washed with Wash buffer, and 50 μL of the suspension was distributed to a 1.5 mL microcentrifuge tube per sample. For each sample, Ago2 antibody-bound beads were incubated with 500 μg soluble tissue lysate in lysis buffer at final volume of 250 μL per sample at 4° C., overnight on rotating mixer. After incubation, beads in each tube were washed 5 times with 1 mL ice cold Wash buffer. Final resuspension of beads was with 50 μL PBST (phosphate buffered saline pH 7.4, 0.25% Triton X-100) per sample. siRNAs were released from bead by heating at 95° C., 5 min. Ago2 TP eluate supernatants were recovered and kept on ice on the same day or stored at −80° C. until the subsequent stem loop-reverse transcription quantitative polymerase chain reaction (SL-RT-qPCR) step.

siRNA Standard Preparation:

siRNA was first diluted to a working stock of 10 ng/μL in H2O, then further diluted 100-fold to 100 ng/mL in PBST. siRNA standards were prepared by 10-fold serial dilution from 100 ng/mL to 0.00001 ng/mL in PBST.

### Stem Loop-Reverse Transcription Quantitative Polymerase Chain Reaction (SL-RT-qPCR):

For SL-RT-qPCR, Custom Small RNA Assay (4398987, ThermoFisher, Waltham MA) containing a set of SL-RT primer and Taqman qPCR primer against guide strand sequence was designed and ordered and cDNA was generated following manufacturer's protocol of the Taqman MicroRNA Reverse Transcription Kit (ThermoFisher, Waltham MA), using 5 μL of Ago2 IP eluate or 5 μL siRNA standard. The cDNA generated (15 μL reaction) were then diluted with 75 μL H2O prior to usage for qPCR step. qPCR was performed following manufacturer's protocol for TaqMan™ Fast Advanced Master Mix (ThermoFisher, Waltham MA), using 4 μL of the diluted cDNA.

### Calculations:

An siRNA standard curve was generated by plotting Ct values (Y) versus siRNA concentration (X) in log scale using GraphPad Prism (version 9.4.1), followed by semi-log line fitting to determine slope and y-intercept values. siRNA concentration for each sample was calculated using the obtained Ct value and the determined slope and y-intercept values.
ng siRNA=[siRNA]×Ago2 IP elution volume (50 μL)
ng siRNA per g soluble lysate protein=ng siRNA per 500 μg (amount used in Ago2 IP)

Data analysis: Statistical significance was determined by ordinary one-way ANOVA and Dunnett's multiple comparisons test using GraphPad Prism software (version 9.4.1).

### 11.3 Results

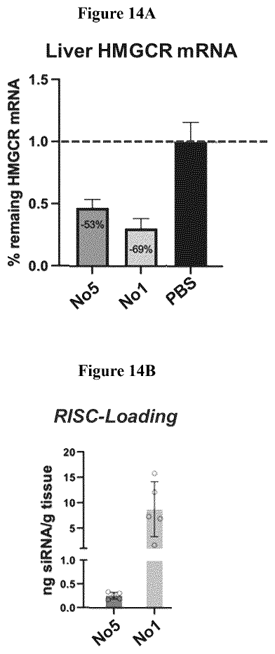

Consistent with the results in Example 4, siRNA Nol containing MOE clamps showed stronger HMGCR mRNA silencing effect than the siRNA No5 without MOE clamps at Day 35 (FIG. 14A). Likewise, siRNA Nol with MOE clamps showed increased higher RISC loading compared to siRNA No5 (FIG. 14B).

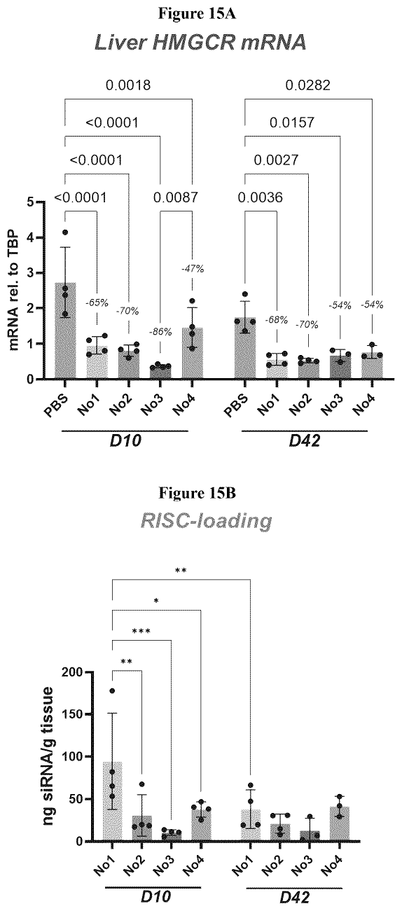

In addition, when the 2′-MOE clamps were replaced with TNA clamps in the same sequence (siRNA No3 and No4), similar HMGCR silencing effects were shown in both Day 10 and Day 42 compared to siRNAs Nol and No2 (FIG. 15A). siRNAs with TNA clamps (No3 and No4) also showed comparable RISC-loading as MOE clamps (siRNAs Nol and No2) in Day 42 (FIG. 15B).

### Example 12: Additional HMGCR siRNAs Tested In Vitro

### 12.1 Methods

Sample siRNAs tested in Example 12 are prepared as described above (e.g., Example 1). The siRNA agents in Tables 15-16 were conjugated with X2000 ligand, and specifically, each 3′-end of the sense strand of each siRNA was conjugated to the XC2000 ligand via phosphodiester linkage.

### 12.1.1 Luciferase Reporter Assay

Hepa-1-6 cells were seeded at 20,000 cells/well in a 96-well plate. At the same time, 100ng/well of reporter plasmid were transfected; the reporter plasmids containing part of HMGCR (1-2438 or 2251-4530 for human HMGCR NCBI NM_000859.3). The reporter plasmid was co-transfected with siRNA 7-point dose response, starting at 6.7 nM with a dilution factor of 1:6 using lipofecamine (Invitrogen, Lipofectamine 2000, cat #11668500). 24 hrs post-transfection, cells were lysed, and luciferase activity was measured based on the manufacturer's protocol (Promega, DualGlow, cat #E2910). The luciferase signal was normalized to Firefly signal to calculate 00 remaining luciferase.

### 12.2 Results

HMGCR siRNAs in Table 15 were tested using the luciferase reporter assay described above and IC50, max. inhibition values and AUC from reporter assay in Hepa-1-6 cells are shown (Table 15).

TABLE 15
IC50, max. inhibition values and AUC
from reporter assay in Hepa-1-6 cells

| | | | | max. |
|---|---|---|---|---|
| siRNA | position | IC50 (nM) | AUC | inhib. [%] |
| 709 | 110 | 0.004296 | 0.0255745 | 97.713 |
| 710 | 111 | 0.006191 | 0.0293495 | 97.463 |
| 712 | 115 | 0.002148 | 0.0295688 | 96.623 |
| 711 | 115 | 0.003058 | 0.0387679 | 96.806 |
| 727 | 115 | 0.003682 | 0.0348478 | 96.992 |
| 713 | 126 | 0.002640 | 0.0266422 | 97.717 |
| 729 | 126 | 0.003042 | 0.0295515 | 96.906 |
| 647 | 126 | 0.005300 | 0.0247178 | 97.434 |
| 728 | 2835 | 0.006231 | 0.0646743 | 94.267 |
| 717 | 2835 | 0.008409 | 0.0722428 | 94.024 |
| 716 | 2835 | 0.016019 | 0.0876096 | 93.002 |
| 714 | 2843 | 0.020361 | 0.0919948 | 92.960 |
| 730 | 2843 | 0.024391 | 0.0945989 | 93.228 |
| 715 | 2843 | 0.030010 | 0.0936334 | 92.498 |
| 720 | 3277 | 0.004929 | 0.0845796 | 93.216 |
| 731 | 3277 | 0.004950 | 0.0740606 | 94.342 |
| 718 | 3277 | 0.004987 | 0.0763132 | 94.409 |
| 722 | 3418 | 0.007055 | 0.1014623 | 93.751 |
| 732 | 3418 | 0.009877 | 0.0780560 | 93.745 |
| 721 | 3418 | 0.030652 | 0.0836624 | 92.941 |

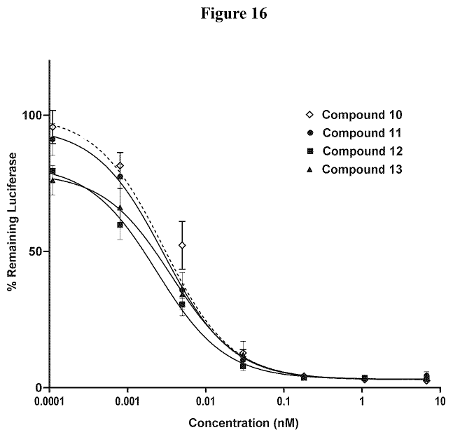

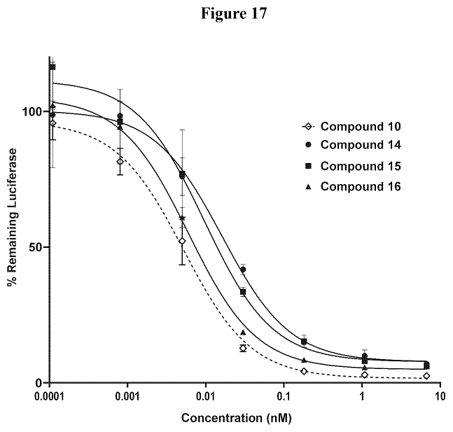

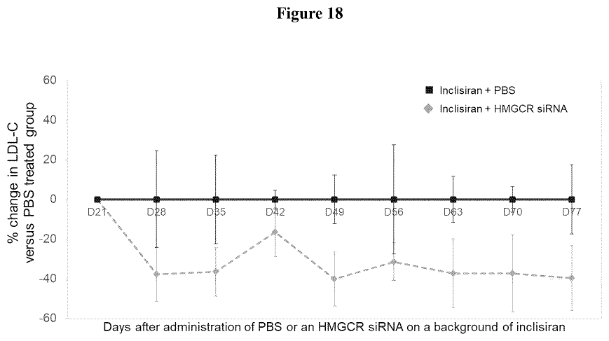

Among others, dose dependent the luciferase reporter assay for siRNAs targeting position 115 and 2835 (listed in Table 16) are shown in FIGS. 16 and 17, respectively.

TABLE 16
Compound list in FIGS. 16 and 17

| | | Sequence | |
|---|---|---|---|
| Compound | | Sense Strand | |
| No. | position | Antisense Strand | Ligand |
| 10 | 126 | SEQ ID NO: 1294 | X2000 |
| | | SEQ ID NO: 1298 | |
| 11 | 115 | SEQ ID NO: 1450 | X2000 |
| | | SEQ ID NO: 1465 | |
| 12 | 115 | SEQ ID NO: 1451 | X2000 |
| | | SEQ ID NO: 1466 | |
| 13 | 115 | SEQ ID NO: 1451 | X2000 |
| | | SEQ ID NO: 2600 | |
| 14 | 2835 | SEQ ID NO: 1455 | X2000 |
| | | SEQ ID NO: 1470 | |
| 15 | 2835 | SEQ ID NO: 1456 | X2000 |
| | | SEQ ID NO: 1471 | |
| 16 | 2835 | SEQ ID NO: 1456 | X2000 |
| | | SEQ ID NO: 2601 | |

As shown in Table 15 and FIGS. 16-17, siRNAs described herein (e.g., Compounds 11 to 16) targeting positions 115 and 2835 of HMGCR mRNA exhibited similar inhibition of HMGCR expression levels as Compound 10 targeting position 126 of HMGCR mRNA. Therefore, they can be potential dsRNAi agents for inhibiting HMGCR expression.

### Example 13: Combination Therapy with Inclisiran

Male cynomolgus monkeys (n=10) were administered a single 3 mg/kg subcutaneous (SC) dose of inclisiran on Day 0. On Day 21 after the inclisiran dose, animals were separated into two groups. One group received a single 5 mg/kg SC dose of an HMGCR siRNA of Table 4 (Compound 1) (n=6), while the other group received a single dose of PBS. Blood samples were collected at multiple times prior to Day 0 and weekly thereafter through Day 77 for the determination of plasma low density lipoprotein (LDL-C) levels. Data are expressed as a percent change in plasma LDL-C versus the group that received inclisiran on Day 0 and PBS on Day 21. Animals in the group that received inclisiran on Day 0 and the HMGCR siRNA on Day 21 showed a nearly 40% further reduction in plasma LDL-C concentrations compared with animals on a background of inclisiran that received PBS on Day 21.

What is claimed:

1. A double stranded RNAi (dsRNAi) agent comprising:
a sense strand comprising a nucleotide sequence selected from SEQ ID NOs: 1 to 405 and 1434 to 1440 in Table 1; and
an antisense strand forming a duplex with the sense strand and comprising a nucleotide sequence selected from SEQ ID NOs: 406 to 810 and 1441 to 1447 in Table 1.

2. The dsRNAi agent of claim 1, wherein the sense strand is 21 to 23 nucleotides in length and the antisense strand is 23 to 25 nucleotides in length.

3. The dsRNAi agent of claim 1 or 2, wherein all the nucleotides in the sense strand and the antisense strand are modified nucleotides.

4. The dsRNAi agent of claim 1 through 3, wherein each of the modified nucleotides independently comprises one or more modifications selected from a 2′-deoxy modification, a 2′-O-alkyl modification, a 2′-halo modification, a threofuranosyl nucleotide (TNA) modification, a 2′-5′-linkage modification, a conformationally restricting modification, an abasic modification, a 2′-amino-modification, a 2′-O-allyl modification, 2′-C-alkyl modification, a 2′-O-alkoxyalkyl modification, a morpholino modification, a phosphoramidate modification, a non-natural nucleobase modification, a modification in a tetrahydropyran, a modification containing a 1,5-anhydrohexitol, a modification containing a cyclohexenyl, a modification containing a phosphorothioate group, a modification containing a 5′-vinyl-phosphonate, a modification containing a 5′-phosphate, a modification to form a thermally destabilizing nucleotide, a glycol nucleic acid (GNA) modification, and a 2-O-(N-methylacetamide) modification.

5. The dsRNAi agent of claim 4, wherein each of the modified nucleotides independently comprises one or more modifications selected from 2′-deoxy modification, 2′-O-alkoxyalkyl modification, 2′-O-alkyl modification, 2′-O-allyl modification, 2′-C-allyl modification, 2′-halo modification, modification containing a non-natural nucleobase, GNA modification, and TNA modification.

6. The dsRNAi agent of any one of claims 3 through 5, wherein all the modified nucleotides comprise a modification on a 2′ sugar ring.

7. The dsRNAi agent of claim 6, wherein the modified nucleotides are selected from a 2′-O-alkyl modified nucleotide, a 2′-halo modified nucleotide, a 2′-deoxy modified nucleotide, a 2′-O-alkoxyalkyl modified nucleotide and TNA modification.

8. The dsRNAi agent of any one of claims 3 to 7, wherein one or more of the modified nucleotides further comprises a 3′-phosphorothioate (PS) modification.

9. The dsRNAi agent of any one of claims 4 through 8, wherein each of the modified nucleotides independently comprises one or more modifications selected from 2′-deoxy modification, 2′-O-methyl (2′-OMe) modification, 2′-fluoro (2′-F) modification, 2′-O-methoxyethyl (2′-MOE) modification, the modification containing a non-natural nucleobase, TNA, GNA, 3′-phosphorothioate (PS) modification, and 5′-vinyl-phosphonate (5′-VP) modification.

10. The dsRNAi agent of any one of claims 1 to 9, wherein the sense strand comprises one or two 2′-MOE modified nucleotides positioned at the 1t and/or 2^{nd} nucleotides from the 5′-end of the sense strand.

11. The dsRNAi agent of any one of claims 1 to 10, wherein the sense strand comprises one or two 2′-MOE modified nucleotides positioned at the 1^{st} and/or 2^{nd} nucleotides from the 3′-end of the sense strand.

12. The dsRNAi agent of any one of claims 1 to 9, wherein the sense strand comprises one or two TNAs positioned at the 1^{st} and/or 2^{nd} nucleotides from the 5′-end of the sense strand.

13. The dsRNAi agent of any one of claims 1 to 10, wherein the sense strand comprises one or two TNAs positioned at the 1^{st} and/or 2^{nd} nucleotides from the 3′-end of the sense strand.

14. The dsRNAi agent of any one of claims 1 through 13, wherein the antisense strand comprises a 5′-VP group at the 1^{st} nucleotide from 5′ end of the antisense strand.

15. The dsRNAi agent of any one of claims 1 through 13, wherein the antisense strand comprises a 5′-(E)-VP group at the 1^{st} nucleotide from 5′ end of the antisense strand.

16. The dsRNAi agent of any one of claims 1 through 13, wherein the antisense strand comprises a 5′-(E)-VP-2′-OMe nucleotide at the 1^{st} position from 5′ end of the antisense strand.

17. The dsRNAi agent of any one of claims 1 and 16, wherein each of the sense strand and the antisense strand independently comprises two, three, four, five or six 2′-F modified nucleotides.

18. The dsRNAi agent of any one of claims 1 through 17, wherein the sense strand comprises one or two 3′-PS group at the 1^{st} and/or 2^{nd} nucleotides from 5′-end of the sense strand.

19. The dsRNAi agent of any one of claims 1 through 18, wherein the antisense strand comprises one or two 3′-PS group at the 1^{st} and/or 2^{nd} nucleotides from 5′-end of the antisense strand, and/or one or two 3′-PS group at the 1^{st} and/or 2^{nd} nucleotides from 3′-end of the antisense strand.

20. The dsRNAi agent of any one of claims 1 through 19, wherein the sense strand is 21 nucleotides in length and the antisense strand is 23 nucleotides in length.

21. The dsRNAi agent of claim 20, wherein the sense strand comprises one to four 2′-MOE modified nucleotides positioned at the 1^{st}, 2^{nd}, 20^{th}, and/or 21^{st} nucleotides from the 5′-end of the sense strand.

22. The dsRNAi agent of claim 21, wherein the sense strand comprises only four 2′-MOE modified nucleotides.

23. The dsRNAi agent of any one of claims 21 through 22, wherein the sense strand does not comprise a 2′-MOE modified nucleotide at the 3^{rd} to 19^{th} positions from 5′-end of the sense strand.

24. The dsRNAi agent of claim 20, wherein the sense strand comprises one to four TNAs positioned at the 1^{st}, 2^{nd}, 20^{th}, and/or 21^{st} nucleotides from the 5′-end of the sense strand.

25. The dsRNAi agent of any one of claims 20 through 24, wherein the sense strand comprises two, three, or four 2′-F modified nucleotides positioned at the 7^{th}, 9^{th}, 10^{th}, and/or 11^{th} nucleotide from 5′-end of the sense strand.

26. The dsRNAi agent of claim 25, wherein the sense strand comprises 2′-F modified nucleotides positioned at the 7^{th}, 9^{th}, 10^{th}, and 11^{th} nucleotides from 5′-end of the sense strand.

27. The dsRNAi agent of claim 25 or 26, wherein the remaining nucleotides in the sense strand comprise 2′-OMe modified modification.

28. The dsRNAi agent of any one of claims 20 through 27, wherein the antisense strand comprises a 5′-(E)-VP group at the 1^{st} nucleotide from 5′ end of the antisense strand.

29. The dsRNAi agent of any one of claims 18 through 25, wherein the antisense strand comprises two, three, or four 2′-F modified nucleotides positioned at the 2^{nd}, 6^{th}, 14^{th}, and/or 16^{th} nucleotides from 5′-end of the antisense strand.

30. The dsRNAi agent of claim 29, wherein the antisense strand comprises 2′-F modified nucleotides positioned at the 2^{nd}, 6^{th}, 14^{th}, and 16^{th} nucleotides from 5′-end of the antisense strand.

31. The dsRNAi agent of any one of claims 20 through 30, wherein the antisense strand comprises 2′-F modifications positioned at the 2nd, 6th, 14th, and 16th nucleotides from the 5′ end; and (i) a GNA positioned at the 5^{th} nucleotide from 5′ end, or (ii) a TNA positioned at the 3rd nucleotide from the 5′ end.

32. The dsRNAi agent of any one of claims 28 through 31, wherein the remaining nucleotides in antisense strand comprise 2′-OMe modified modifications.

33. The dsRNAi agent of any one of claims 20 through 32, wherein the sense strand comprises one to eight 3′-PS group at the 1^{st}, 2^{nd}, 3^{rd}, 4^{th}, 17^{th}, 18^{th}, 19^{th} and/or 20^{th} nucleotides from 5′-end of the sense strand.

34. The dsRNAi agent of any one of claims 20 through 33, wherein the antisense strand comprises one to eight 3′-PS group at the 1^{st}, 2^{nd}, 3^{rd}, 4^{th}, 19^{th}, 20^{th}, 21^{st} and/or 22^{nd} nucleotides from 5′-end of the antisense strand.

35. The dsRNAi agent of any one of claims 18, 19, 33 and 34, wherein at least one of the 3′-PS groups in each sense strand and antisense strand has a stereopure Rp configuration.

36. The dsRNAi agent of any one of claims 18, 19, 33 and 34, wherein at least one of the 3′-PS groups in each sense strand and antisense strand has a stereopure Sp configuration.

37. A double stranded RNAi (dsRNAi) agent comprising:
a sense strand having a nucleotide sequence selected from SEQ ID NOs: 812 to 1052 in Table 2 and SEQ ID NOs: 1294 to 1297, 1448 to 1462, and 1481 to 1482 in Table 3; and
an antisense strand forming a duplex with the sense strand and having a nucleotide sequence selected from SEQ ID NOs: 1053 to 1293 in Table 2 and 1298 to 1301, 1463 to 1477, and 2600 to 2605 in Table 3.

38. The dsRNAi agent of any one of claims 1 through 37, further comprising a ligand.

39. The dsRNAi agent of claim 38, wherein the ligand comprises a N-acetylgalactosamine (GalNAc) moiety.

40. The dsRNAi agent of claim 38 or 39, wherein the ligand has a structure of: 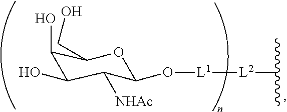
wherein:
each L¹ is independently a linker which may be same or different in each occurrence;
L² is a linker;
n is an integer from 1 to 3; and [symbol]
is an attachment point to the sense strand or an antisense strand.

41. The dsRNAi agent of claim 40, wherein the ligand comprises the following structure of 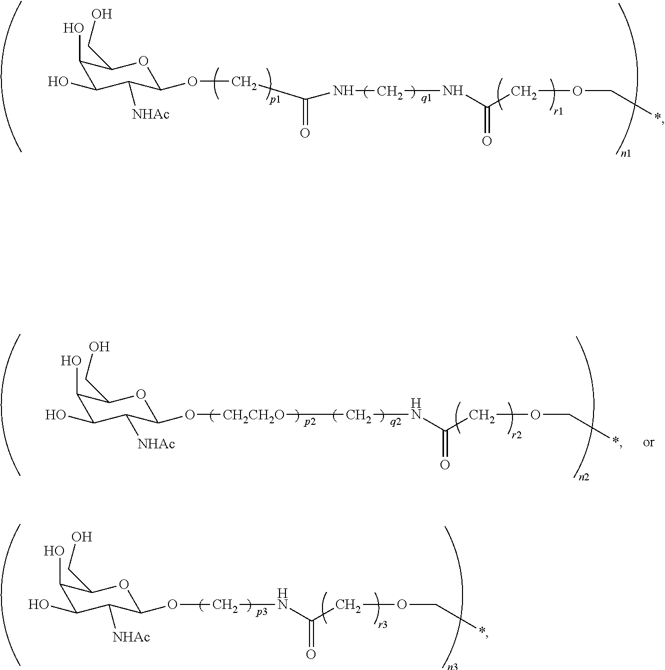
wherein:
each p1, p2, p3, q1, q2, r1, r2 and r3 is independently an integer from 0 to 12;
each n1, n2, and n3 is independently an integer from 1 to 3; and
“*” is an attachment point to L².

42. The dsRNAi agent of claim 38 or 39, wherein the ligand has a structure of: 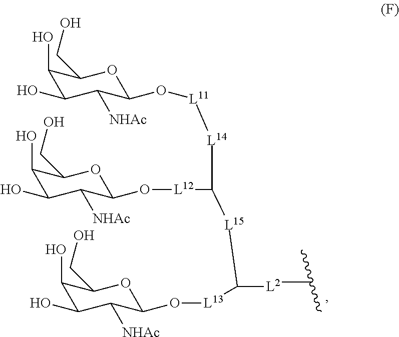
wherein:
each L¹¹, L¹², L¹³, L¹⁴, and L¹⁵ is an independently a linker;
L² is a linker; [symbol]
is an attachment point to the sense strand or the antisense strand.

43. The dsRNAi agent of claim 42, wherein the ligand has a structure of: 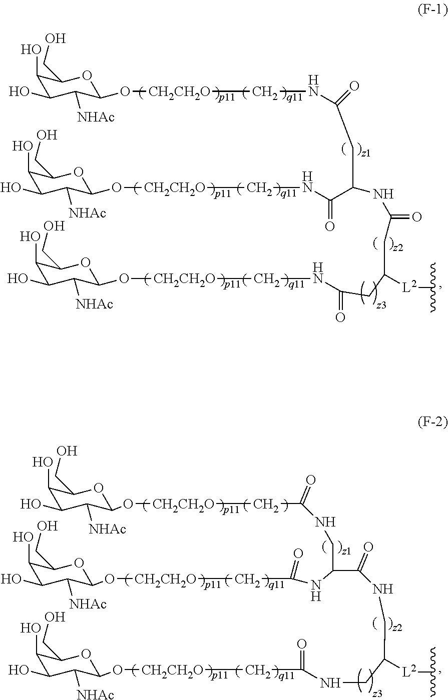
wherein:
each p11 and q11 is independently an integer from 0 to 12;
each z1, z2, and z3 is independently an integer of 0 to 12; and [symbol]
is an attachment point to the sense strand or the antisense strand.

44. The dsRNAi agent of any one of claims 38 through 43, wherein the ligand comprises the following structure: 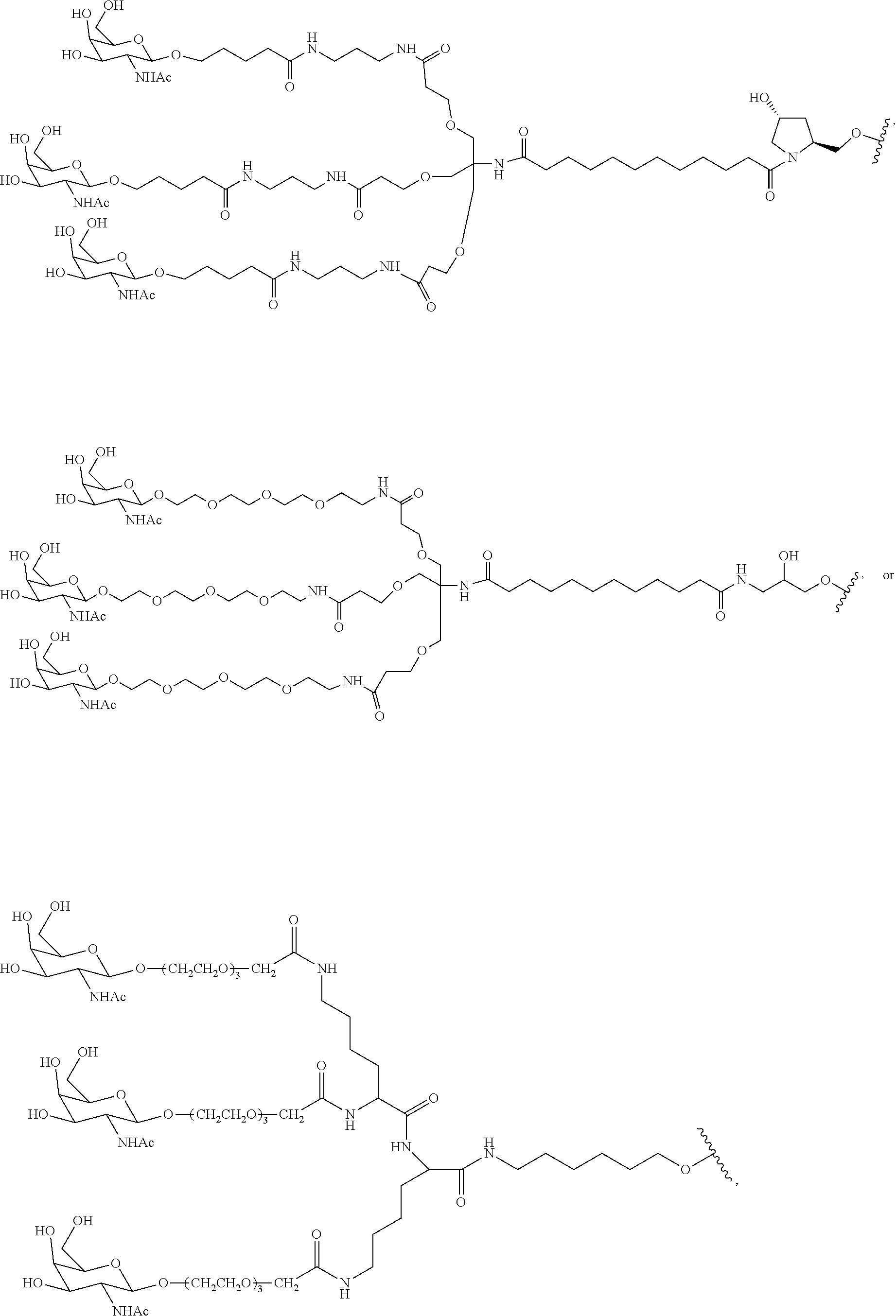
wherein [symbol]
is an attachment point to the sense strand or the antisense strand.

45. The dsRNAi agent of claim 44, wherein the ligand is conjugated to 3′ end of the sense strand to form the following structure: 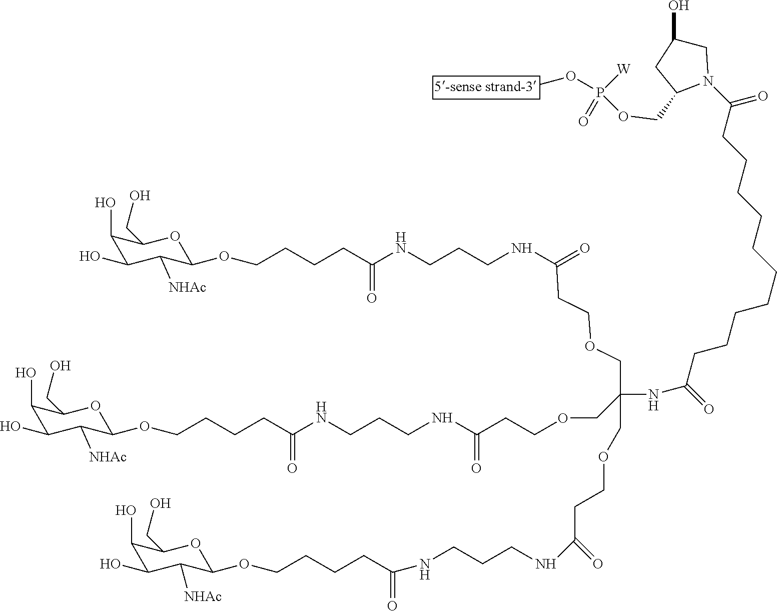
or a pharmaceutically acceptable salt,
wherein W is —OH or —SH.

46. The dsRNAi agent of claim 44, wherein the ligand is conjugated to 5′ end of the sense strand to form the following structure: 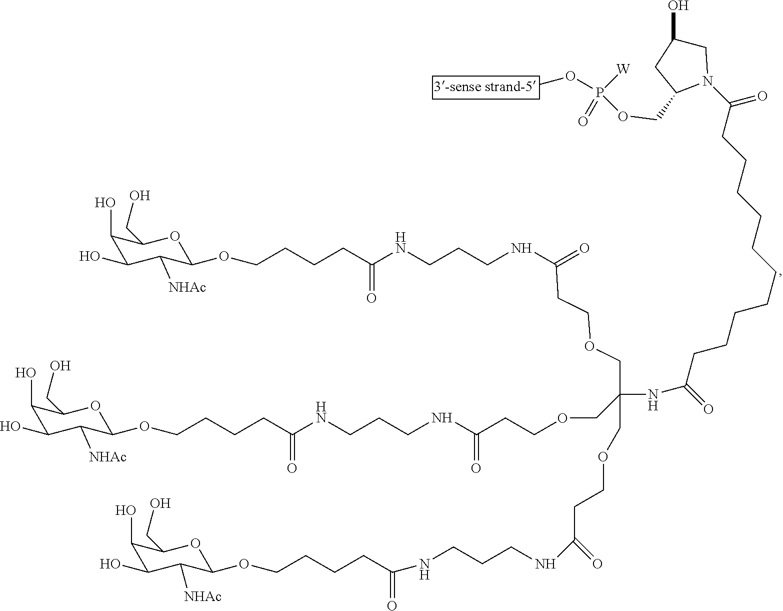
or a pharmaceutically acceptable salt,
wherein W is —OH or —SH.

47. The dsRNAi agent of claim 45 or 46, wherein W is —OH.

48. The dsRNAi agent of any one of claims 1 through 47, wherein the dsRNAi agent is in a pharmaceutically acceptable salt form.

49. The dsRNAi agent of claim 45, wherein the pharmaceutically acceptable salt is a sodium salt.

50. A pharmaceutical composition comprising the dsRNAi agent of any one of claims 1 through 49, and a pharmaceutically acceptable carrier.

51. The pharmaceutical composition of claim 50, wherein the composition is in an aqueous solution form.

52. The pharmaceutical composition of any of claims 50 through 51, further comprising an additional therapeutic agent selected from a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

53. The pharmaceutical composition of claim 52, wherein the additional therapeutic agent comprises a PCSK9 inhibitor.

54. The pharmaceutical composition of claim 53, wherein the PCSK9 inhibitor is a second dsRNAi agent.

55. The pharmaceutical composition of claim 54, wherein the second dsRNAi agent comprises inclisiran.

56. A combination of (i) the dsRNAi agent of any one of claims 1 through 49, and (ii) a second agent selected from a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

57. The combination of claim 56, wherein the second agent is a second dsRNAi agent.

58. The combination of claim 57, wherein the second dsRNAi agent is a dsRNA agent that targets one or more of the genes selected from the group consisting of PCSK9, LPA, AGT, ACE, ACE2, AGTR1, AGTR2, ACAT, CETP, MTTP, PPAR, IBAT, FDFT1, ERG9, SQS1, Ccl2, CCR2, CCL7, CCL8, CCL13, and CCL16.

59. The combination of any one of claims 56 to 58, wherein the second dsRNAi agent comprises inclisiran.

60. A pharmaceutical composition comprising the combination of any one of claims 56 through 59.

61. The pharmaceutical composition of claim 60, wherein the second dsRNAi agent is in a pharmaceutically acceptable salt form.

62. The pharmaceutical composition of claim 61, wherein the pharmaceutically acceptable salt of the second dsRNAi agent is a sodium salt.

63. The pharmaceutical composition of any one of claims 60 to 62, wherein the dsRNAi agent and the second agent are formulated in the same composition.

64. The pharmaceutical composition of any one of claims 60 to 63, wherein the dsRNAi agent and the second agent are formulated in the separate compositions.

65. A method of inhibiting expression of 3-hydroxy-3-methylglutaryl-CoA reductase (HMGCR) in a subject comprising:
administering to the subject the dsRNAi agent of any one of claims 1 through 49 or a pharmaceutically acceptable salt thereof, or the pharmaceutical composition of any one of claims 50 through 55.

66. A method of lowering a level of low-density lipoprotein cholesterol (LDL-C) in a subject, comprising:
administering to the subject the dsRNAi agent of any one of claims 1 through 49 or a pharmaceutically acceptable salt thereof, or the pharmaceutical composition of any one of claims 50 through 55.

67. A method of treating or preventing an HMGCR-associated disorder or disease in a subject, comprising:
administering to the subject the dsRNAi agent of any one of claims 1 through 49 or a pharmaceutically acceptable salt thereof, or the pharmaceutical composition of any one of claims 50 through 55.

68. The method of claim 67, wherein the HMGCR-associated disorder or disease is hyperlipidemia, hypercholesterolemia, hypertriglyceridemia, mixed hyperlipidemia, primary hyperlipidemia, heterozygous familiar hypercholesterolemia (HeFH), homozygous familiar hypercholesterolemia (HoFH), congestive heart disease (CHD) or atherosclerosis.

69. A method of treating or preventing hyperlipidemia in a subject, comprising:
administering to the subject the dsRNAi agent of any one of claims 1 through 49 or a pharmaceutically acceptable salt thereof, or the pharmaceutical composition of any one of claims 50 through 55.

70. The method of claim 69, wherein the hyperlipidemia is hypercholesterolemia, or hypertriglyceridemia.

71. A method of treating or preventing atherosclerotic cardiovascular disease (ASCVD) in a subject, comprising:
administering to the subject the dsRNAi agent of any one of claims 1 through 49 or a pharmaceutically acceptable salt thereof, or the pharmaceutical composition of any one of claims 50 through 55.

72. The method of any one of claims 65 through 71, wherein the dsRNAi agent or the pharmaceutical composition is administered subcutaneously or intravenously.

73. The method of any one of claims 62 through 72, further comprising administering to the subject an additional therapeutic agent selected from a proprotein convertase subtilisin kexin 9 (PCSK9) inhibitor, a lysophosphatidic acid (LPA) receptor inhibitor, an angiotensinogen (AGT) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

74. The method of claim 73, wherein the additional therapeutic agent is a second dsRNAi agent.

75. The method of claim 74, wherein the second dsRNAi agent comprises a PCSK9 inhibitor.

76. The method of claim 75, wherein the second dsRNAi agent comprises inclisiran.

77. The method of any one of claims 73 through 76, wherein the dsRNAi agent or the pharmaceutical composition and the additional therapeutic agent are administered simultaneously.

78. The method of any one of claims 73 through 76, wherein the dsRNAi agent or the pharmaceutical composition and the additional therapeutic agent are administered subsequently.

79. The method of claim 78, wherein the dsRNAi agent is administered before administering the additional therapeutic agent.

80. The method of claim 78, wherein the additional therapeutic agent is administered before administering the dsRNAi agent.

81. The method of any one of claims 73 through 80, wherein the additional therapeutic agent is administered subcutaneously or intravenously.

82. The method of any one of claims 65 through 81, wherein the subject is a human.

83. The method of any one of claims 65 through 82, wherein the subject has or is diagnosed with hyperlipidemia, hypercholesterolemia, hypertriglyceridemia, mixed hyperlipidemia, primary hyperlipidemia, heterozygous familiar hypercholesterolemia (HeFH), homozygous familiar hypercholesterolemia (HoFH), congestive heart disease (CHD) or atherosclerosis.

84. The method of any one of claims 65 through 83, wherein the subject does not have a muscle side effect after the administrating the dsRNAi agent of any one of claims 1 through 49 or a pharmaceutically acceptable salt thereof, or the pharmaceutical composition of any one of claims 50 through 55.

85. A method of lowering a level of low-density lipoprotein cholesterol (LDL-C) in a subject, comprising:
administering to the subject the pharmaceutical composition of any one of claims 60 through 64.

86. A method of treating or preventing an HMGCR-associated disorder or disease in a subject, comprising:
administering to the subject the pharmaceutical composition of any one of claims 60 through 64.

87. The method of claim 86, wherein the HMGCR-associated disorder or disease is hyperlipidemia, hypercholesterolemia, hypertriglyceridemia, mixed hyperlipidemia, primary hyperlipidemia, heterozygous familiar hypercholesterolemia (HeFH), homozygous familiar hypercholesterolemia (HoFH), congestive heart disease (CHD) or atherosclerosis.

88. A method of treating or preventing hyperlipidemia in a subject, comprising:
administering to the subject the pharmaceutical composition of any one of claims 60 through 64.

89. A method of treating or preventing atherosclerotic cardiovascular disease (ASCVD) in a subject, comprising:
administering to the subject the pharmaceutical composition of any one of claims 60 through 64.

90. The method of any one of claims 85 through 89, wherein the dsRNAi agent and the second agent is administered subcutaneously or intravenously.

91. The method of any one of claims 85 through 89, wherein the dsRNAi agent and the second agent are administered simultaneously.

92. The method of any one of claims 85 through 91, wherein the dsRNAi agent and the second agent are administered subsequently.

93. The method of claim 92, wherein the dsRNAi agent is administered before administering the second agent.

94. The method of claim 92, wherein the second agent is administered before administering the dsRNAi agent.

95. The method of any one of claims 85 through 94, wherein the subject is a human.

96. The method of any one of claims 85 through 95, wherein the subject has or is diagnosed with hyperlipidemia, hypercholesterolemia, hypertriglyceridemia, mixed hyperlipidemia, primary hyperlipidemia, heterozygous familiar hypercholesterolemia (HeFH), homozygous familiar hypercholesterolemia (HoFH), congestive heart disease (CHD) or atherosclerosis.

97. The method of any one of claims 85 through 96, wherein the subject does not have a muscle side effect after the administrating the pharmaceutical composition of any one of claims 60 through 64.

98. A method of reducing the risk of a major adverse cardiovascular event in a subject, comprising administering to the subject the dsRNAi agent of any one of claims 1 through 49 or a pharmaceutically acceptable salt thereof, the pharmaceutical composition of any one of claims 50 through 55, the combination of any one of claims 56 through 59, or the pharmaceutical composition of any one of claims 60 through 64.

99. The method of claim 98, wherein the major adverse cardiovascular event is cardiovascular death, non-fatal myocardial infarction, non-fatal ischemic stroke, or urgent coronary revascularization.

100. The method of claim 98, wherein the subject has an established cardiovascular disease.

101. The method of claim 98, where the subject has not experienced a major atherosclerotic cardiovascular disease (ASCVD) event.

102. A kit comprising the dsRNAi agent of any one of claims 1 through 49 or a pharmaceutically acceptable salt thereof, or the pharmaceutical composition of any one of claims 50 through 55.

103. The kit of claim 102, further comprising an additional therapeutic agent selected from a proprotein convertase subtilisin kexin 9 (PCSK9) inhibitor, a fibrate, a bile acid sequestrant, niacin, an antiplatelet agent, an angiotensin converting enzyme inhibitor, an angiotensin II receptor antagonist, an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant, a glycoprotein IIb/IIIa inhibitor, aspirin or an aspirin-like compound, an IBAT inhibitor, a squalene synthase inhibitor, a monocyte chemoattractant protein (MCP)-I inhibitor, and a combination thereof.

104. The kit of claim 103, wherein the additional therapeutic agent is a second dsRNAi agent.

105. The kit of claim 104, wherein the second dsRNAi agent is a dsRNA agent that targets one or more of the genes selected from the group consisting of PCSK9, LPA, AGT, ACE, ACE2, AGTR1, AGTR2, ACAT, CETP, MTTP, PPAR, IBAT, FDFT1, ERG9, SQS1, Ccl2, CCR2, CCL7, CCL8, CCL13, and CCL16.

106. The kit of claim 105, wherein the second dsRNAi agent comprises the PCSK9 inhibitor.

107. The kit of claim 106, wherein the second dsRNAi agent comprises inclisiran.

108. The kit of any one of claims 103 through 107, wherein the dsRNAi agent and the additional therapeutic agent are contained in a single vial.

109. The kit of any one of claims 103 through 107, wherein the dsRNAi agent and the additional therapeutic agent are contained in separate vials.

110. The kit of any one of claims 102 through 109, further comprising one or more applicators.

111. The kit of claim 110, wherein the one or more applicators comprises a syringe.

112. A kit comprising the pharmaceutical composition of any one of claims 60 through 64.

113. The kit of claim 112, wherein the dsRNAi agent and the second agent are contained in a single vial.

114. The kit of claim 112, wherein the dsRNAi agent and the second agent are contained in separate vials.

115. The kit of any one of claims 103 through 114, further comprising one or more applicators.

116. The kit of claim 115, wherein the one or more applicators are syringes.
